(19) United States
(12) Patent Application Publication
(10) Pub. No.: US 2025/0136568 A1
(43) Pub. Date: May 1, 2025

(54) FUSED HETEROCYCLIC COMPOUNDS AS PI3KALPHA INHIBITORS

(71) Applicant: Nanjing Zenshine Pharmaceuticals Co., Ltd., Nanjing (CN)

(72) Inventors: Xiaolin HAO, Foster City, CA (US); Yonghua LEI, Nanjing (CN)

(21) Appl. No.: 18/716,156

(22) PCT Filed: Dec. 7, 2022
(86) PCT No.: PCT/CN2022/137307

§ 371 Date: Jun. 4, 2024

(30) Foreign Application Priority Data
Dec. 8, 2021 (WO) PCT/CN2021/136336
May 30, 2022 (WO) PCT/CN2022/095857
Sep. 30, 2022 (WO) PCT/CN2022/123120

(51) Int. Cl. C07D 311/32; A61K 31/353; A61K 31/357; A61K 31/381; A61K 31/4025; A61K 31/4035; A61K 31/404; A61K 31/4155; A61K 31/416; A61K 31/4178; A61K 31/4184; A61K 31/4192; A61K 31/4196; A61K 31/422; A61K 31/423; A61K 31/4245; A61K 31/427; A61K 31/437; A61K 31/4433; A61K 31/4439; A61K 31/453; A61K 31/4545; A61K 31/4709; A61K 31/4725; A61K 31/496; A61K 31/4985; A61K 31/502; A61K 31/506; A61K 31/517; A61K 31/5377; A61K 31/541; A61K 31/675; C07D 405/04; C07D 405/10; C07D 405/12; C07D 405/14; C07D 407/04; C07D 409/04; C07D 417/04; C07D 471/04; C07D 487/04; C07F 9/6558

(52) U.S. Cl. CPC C07D 311/32; A61K 31/353; A61K 31/357; A61K 31/381; A61K 31/4025; A61K 31/4035; A61K 31/404; A61K 31/4155; A61K 31/416; A61K 31/4178; A61K 31/4184; A61K 31/4192; A61K 31/4196; A61K 31/422; A61K 31/423; A61K 31/4245; A61K 31/427; A61K 31/437; A61K 31/4433; A61K 31/4439; A61K 31/453; A61K 31/4545; A61K 31/4709; A61K 31/4725; A61K 31/496; A61K 31/4985; A61K 31/502; A61K 31/506; A61K 31/517; A61K 31/5377; A61K 31/541; A61K 31/675; C07D 405/04; C07D 405/10; C07D 405/12; C07D 405/14; C07D 407/04; C07D 409/04; C07D 417/04; C07D 471/04; C07D 487/04; C07F 9/65586

(57) ABSTRACT

The present disclosure describes antagonists of the enzyme. The present disclosure provides compounds having a structure set forth in Formula (VI), (VII), (VII-1) or (VIII), the stereoisomer or pharmaceutically acceptable salts, solvates and prodrugs thereof. Further provided are uses of the compounds of Formula (VI), (VII), (VII-1) or (VIII) in treating cancers.

### FIELD OF THE INVENTION

This invention pertains to fused heterocyclic compounds that inhibit the mutated phosphoinositide 3-kinase a (PI3Kα) and are useful for the treatment of diseases and conditions such as cancer mediated by PI3Kα mutations.

### BACKGROUND OF THE INVENTION

Protein kinases are enzymes crucial for multiple fundamental biological processes and complex functions such as regulation of the cell cycle and signal transduction. The phosphoinositide 3-kinase (PI3K) is the family member of intracellular signal transducer enzymes that catalyze the phosphorylation of the 3-position hydroxyl group of the inositol ring of phosphatidylinositols. PI3K signaling is involved in many disease states including cancer and inflammatory diseases such as allergic contact dermatitis and rheumatoid arthritis et, al., which makes PI3Ks become important therapeutic targets in recent decades.

PI3Ks are divided into three classes: class I, II, and III. Class I PI3Ks are further classified into four isoforms: PI3Kα, PI3Kβ, PI3Kγ, and PI3Kδ (Fruman, et, al., Phosphoinositide kinases. Anu. Rev. Biochem. 1998, 67, 481-507). The class I PI3Ks are typically activated by tyrosine kinases or G-protein coupled receptors to generate PIP3, which engages downstream effectors such as those in the pathways of Akt/PDK1, mTOR, the Tec family kinases, and the Rho family GTPases.

The PI3K isoforms have been implicated in a variety of human cancers and disorders and become as one of the most highly mutated systems in human cancers. Mutations in the gene coding for a PI3K isoform are point mutations clustered within several hotspots in helical and kinase domains. Because of the high rate of PI3K mutations, targeting of the mutations in this pathway may provide valuable therapeutic opportunities.

PI3Kα isoform is of particular pharmacological target of interest since the over-activation of PI3Kα signaling is one of the most frequent events leading to cancer. Deregulation of the PI3Kα pathway may occur by several different mechanisms, including somatic mutations and amplification of genes encoding key components of the PI3Kα pathway (Courtney, K. D. et, al., The PI3K pathway as drug target in human cancer J. Clin. Oncol. 2010, 28, 1075-1083.). In a variety of human cancers, such as lung, stomach, endometrial, ovarian, bladder, breast, colon, brain, prostate, and skin, mutations in the gene coding for PI3Kα or mutations which lead to upregulation of PI3Kα are believed to occur within several hotspots in helical and kinase domains, such as E542K, E545K and H1047R, of which H1047R substitution in the kinase domain close to the C terminus and a cluster of three charge-reversal mutations (E542K, E545K, Q546K) in the helical domain of p110α counts for 80%.

PIK3CA is mutated in 27% of breast cancer cases, 24% in endometrial cancer and 15% in colon cancer (Lin, Pet, al., Targeting the phosphoinositide 3-kinase pathway in cancer Nat. Rev, Drug Discovery. 2009, 8, 627-644.). Due to its key role in regulating organismal glucose homeostasis, inhibition by pan PI3K inhibitors or PI3Kα inhibitors which are potent against both mutant PI3Kα and wild-type PI3Kα often causes hyperglycemia and/or hyperinsulinemia (Busaidy N L, et al, Management of metabolic effects associated with anticancer agents targeting the P3K-Akt-mTOR pathway. J Clin Oncol 2012, 30, 2919-28). Hyperglycemia may be worsened or prolonged in insulin resistance patients and therefor results discontinuation of PI3K inhibitor treatment (Hopkins, B, et al, Suppression of insulin feedback enhances the efficacy of PI3K inhibitors, Nature, 2018, 560, 499-503).

Enhancing selectivity for mutant PI3Kα over wild-type PI3Kα can potentially provide an increased window for complete inhibition of the pathologic signaling of mutant PI3Kα in the cancer cells while limiting toxicities caused by affecting the wild-type PI3Kα in the host tissues that control systemic metabolism (Okkenhaug K, et, al., Targeting PI3K in Cancer: Impact on Tumor Cells, Their Protective Stroma, Angiogenesis, and Immunotherapy. Cancer Discov. 2016 Oct. 6(10):1090-1105).

Known PI3Kα inhibitors currently are nearly equipotent to wild-type and mutant PI3Kα. As the PI3Kα mutations are located far from the active site, the development of allosteric inhibitors targeting binding pocket near a known mutation (e.g, H1047R) may provide a route to selective PI3Kα inhibition. Allosteric inhibitors have a number of potential benefits over the canonical ATP competitive inhibitors, such as greater selectivity due to the less conserved binding site.

The present disclosure provides a new class of kinase inhibitors to PI3Kα mutations.

### SUMMARY OF THE INVENTION

The objective of the present invention is to provide a compound of formula (VI), a stereoisomer thereof, or a pharmaceutically acceptable salt, solvates and prodrugs thereof:

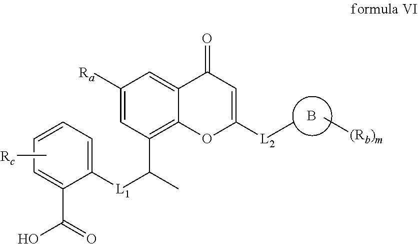

Wherein,
L₁ is selected from —O—(CHR₁)ₚ—, —N(R₁₁)—(CHR₁)ₚ—;
R₁ is identical or different and each is independently selected from hydrogen, deuterium, C₁-C₆ alkyl, C₁-C₆ haloalkyl, C₁-C₆ alkoxy, C₁-C₆ haloalkoxy, halogen, hydroxy, cyano;
R₁₁ is identical or different and each is independently selected from hydrogen, C₁-C₆ alkyl, C₁-C₆ haloalkyl;
Rₐ is independently selected from the group consisting of hydrogen, deuterium, C₁-C₆ alkyl, deuterated C₁-C₆ alkyl, C₁-C₆ haloalkyl, C₁-C₆ alkoxy, C₁-C₆ haloalkoxy, halogen, amino, nitro, hydroxy, cyano, —C(O)N(Rₐₐ)₂, C₃-C₈ cycloalkyl, 4 to 7 membered heterocyclyl, C₆-C₁₀ aryl and 5 to 10 membered heteroaryl, wherein the C₃-C₈ cycloalkyl, 4 to 7 membered heterocyclyl, C₆-C₁₀ aryl and 5 to 10 membered heteroaryl are each optionally further substituted by one or more deuterium, C₁-C₆ alkyl, halogen, hydroxy, amino, nitro, cyano, ester group, C₁-C₆ alkoxy; Each Rₐₐ is identical or different and is each independently selected from hydrogen or C₁-C₆ alkyl, wherein the C₁-C₆ alkyl optionally further substituted by one or more substituents selected from the group consisting of deuterium, halogen, hydroxy, amino, —N(C₁-C₆ alkyl)₂;

Or two Rₐₐ together with the atoms to which they are attached can form a 4 to 7 membered heterocycle comprising 1-2 heteroatoms selected from O, N, and S, wherein optionally substituted with one or more one or more oxo, halogen, —CN, —OH, —NH₂, C₁-C₆ alkyl, C₁-C₆ haloalkyl or C₁-C₆ alkoxy;

While L₂ is a bond or S;

Ring B is independently selected from

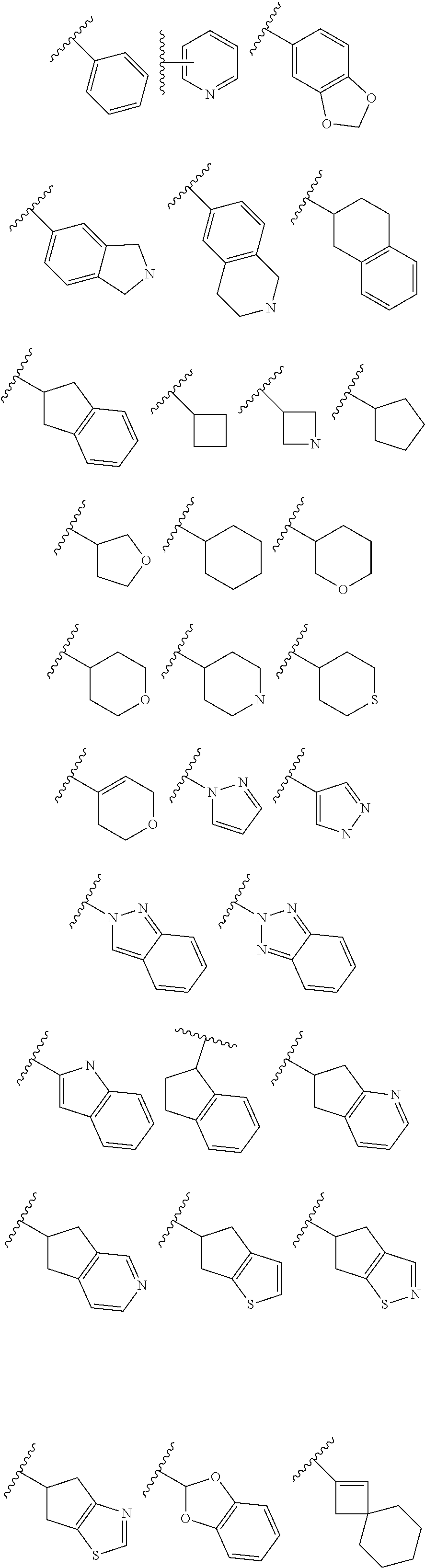

While L₂ is CR₂R₂′;

Each R₂ and R₂′ is independently selected from hydrogen, C₁-C₆ alkyl, C₁-C₆ alkoxyl or C₁-C₆ haloalkyl;

Ring B is independently selected from:

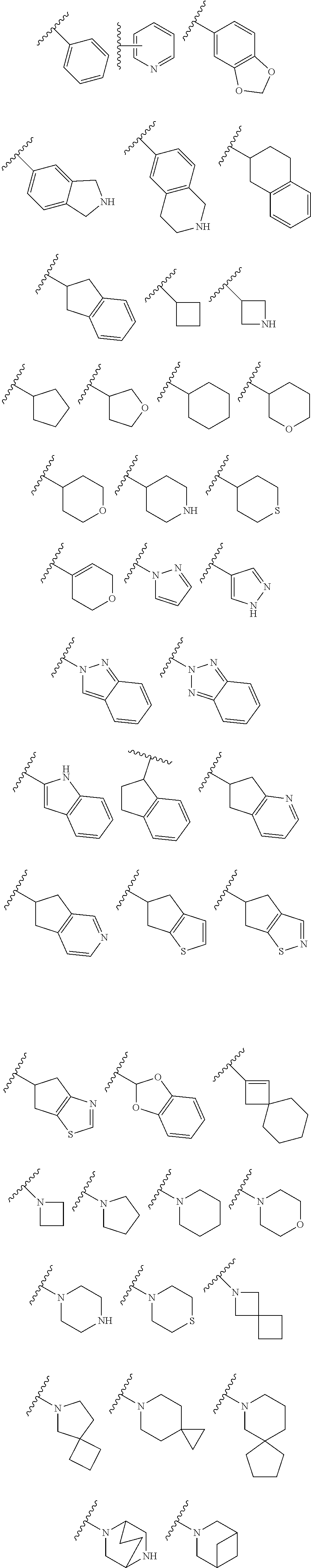

Each R_{b} is identical or different and each is independently selected from the group consisting of hydrogen, oxo, halogen, hydroxy, amino, nitro, cyano, ester group, C₁-C₆ alkyl, C₁-C₆ alkoxy, C₃-C₈ cycloalkyl and 4 to 7 membered heterocyclyl, wherein the C₁-C₆ alky, C₃-C₈ cycloalkyl and 4 to 7 membered heterocyclyl are each optionally further substituted by one or more substituents selected from the group consisting of deuterium, halogen, hydroxy, amino, nitro, cyano, ester group, carboxyl, alkoxy, hydroxyalkyl;
R_{c} is identical or different and is each independently selected from the group consisting of hydrogen, deuterium, C₁-C₆ alkyl, deuterated C₁-C₆ alkyl, C₁-C₆ haloalkyl, C₁-C₆ alkoxy, C₁-C₆ haloalkoxy, —S(O)₂-alkyl, halogen, amino, nitro, hydroxy, cyano, C₃-C₉ cycloalkyl and 4 to 7 membered heterocyclyl, wherein the C₃-C₈ cycloalkyl and 4 to 7 membered heterocyclyl are each optionally further substituted by one or more deuterium, alkyl, halogen, hydroxy, amino, nitro, cyano, ester group, alkoxy;
m is an integer of 0, 1, 2, 3, 4 or 5;
p is an integer of 0, 1 or 2.

In a more preferred embodiment, in the compound of formula (VI), the stereoisomer or the pharmaceutically acceptable salt, solvates and prodrugs thereof, each L₁ is —N(R₁₁)—(CHR₁)ₚ— (such as —NH—,

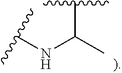 [SMILES: *NC(*)C]

In a more preferred embodiment, in the compound of formula (VI), the stereoisomer or the pharmaceutically acceptable salt, solvates and prodrugs thereof, each Rₐ is independently selected from halogen, C₁-C₆ alkyl (such as methyl, ethyl, propyl and isopropyl), C₃-C₈ cycloalkyl (such as cyclopropyl), C₁-C₆ alkoxy, hydroxy, cyano

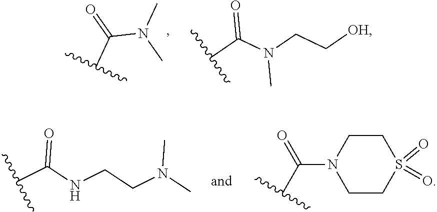

In a more preferred embodiment, in the compound of formula (VI), the stereoisomer or the pharmaceutically acceptable salt, solvates and prodrugs thereof, each R, is independently selected from hydrogen, halogen, oxo, C₁-C₆ alkyl (such as methyl, ethyl, propyl and isopropyl), cyclopropyl, carboxymethyl, C₁-C₆ alkoxy, C₁-C₆ alkyl substituted by one or more halogen (such as 2-fluoro-2-methylpropyl) and 4 to 7 membered heterocyclyl (such as oxetan-3-yl).

In a more preferred embodiment, in the compound of formula (VI), the stereoisomer or the pharmaceutically acceptable salt, solvates and prodrugs thereof, each Re is independently selected from hydrogen, C₁-C₆ alkyl (such as methyl, ethyl, propyl and isopropyl), C₁-C₆ haloalkyl, C₁-C₆ alkoxy, halogen, amino, nitro, hydroxy, cyano and —S(O)-methyl.

In another embodiment, described here are compounds of formula (VII), a stereoisomer thereof, or a pharmaceutically acceptable salt, solvates and prodrugs thereof:

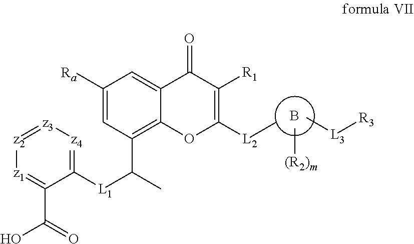

Wherein,
Each Z₁, Z₂, Z₃ and Z₄ is independently selected from CH, N or CR_{c};
R_{c} is each independently selected from hydrogen, deuterium, C₁-C₆ alkyl, deuterated C₁-C₆ alkyl, C₁-C₆ haloalkyl, C₁-C₆ alkoxy, C₁-C₆ haloalkoxy, —S(O)₂-alkyl, halogen, amino, nitro, hydroxy, cyano;
L₁ is independently selected from —N(H)—, —N(C₁-C₆ alkyl)-, —O—, —S— and —C₁-C₂—;
Rₐ is independently selected from hydrogen, deuterium, C₁-C₆ alkyl, deuterated C₁-C₆ alkyl, C₁-C₆ haloalkyl, C₁-C₆ hydroxyalkyl, C₁-C₆ alkoxy, C₁-C₆ haloalkoxy, halogen, amino, nitro, hydroxy, cyano, C₃-C₅ cycloalkyl, 4 to 10 membered heterocyclyl, C₆-C₁₀ aryl and 5 to 10 membered heteroaryl;
R₁ is independently selected from hydrogen, deuterium, halogen, hydroxy, cyano, C₁-C₆ alkyl, deuterated C₁-C₆ alkyl, C₁-C₆ haloalkyl, C₁-C₆ alkoxy C₁-C₆ haloalkoxy, C₃-C₈ cycloalkyl and 5 or 6 membered heteroaryl;
L₂ is a bond,

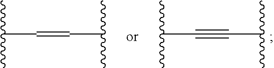

X₁ is a ring atom of ring B and ring B is connected to L₂ through X₁ atom, wherein X₁ is a carbon atom;
Ring B is independently selected from C₃-C₈ cycloalkyl, 4 to 10 membered heterocyclyl, C₆-C₁₀ aryl and 5 to 10 membered heteroaryl;
R₂ is identical or different and each is independently selected from hydrogen, deuterium, oxo, halogen, hydroxy, amino, nitro, cyano, ester group, C₁-C₆ alkyl, deuterated C₁-C₆ alkyl, C₁-C₆ hydroxyalkyl, C₁-C₆ haloalkyl, C₁-C₆ alkoxy, C₃-C₈ cycloalkyl, 4 to 10 membered heterocyclyl, C₆-C₁₀ aryl and 5 to 10 membered heteroaryl, wherein C₃-C₈ cycloalkyl, 4 to 10 membered heterocyclyl, C₆-C₁₀ aryl and 5 to 10 membered heteroaryl are each optionally further substituted by one or more substituents selected from deuterium, halogen, hydroxy, amino, nitro, cyano, ester group, carboxyl, C₁-C₆ alkyl, deuterated C₁-C₆ alkyl, C₁-C₆ hydroxyalkyl, C₁-C₆ haloalkyl and C₁-C₆ alkoxy.
L₃ is independently selected from bond, —O—, —(CH₂)₁₋₄—, —C(O)—, —C(O)N(H)—, —N(H)C(O)—, —C(O)N(C₁-C₆ alkyl)- and —N(C₁-C₆ alkyl)C(O)—, —S(O)₂—, —(CH₂)₁₋₄—C(O)—, —O—(CH₂)₁₋₄—, —CH(C₁-C₆ alkyl)-, C₃-C₈ cycloalkyl, 4 to 10 membered heterocyclyl, C₆-C₁₀ aryl and 5 to 10 membered heteroaryl.

R₃ is independently selected from hydrogen, deuterium, halogen, hydroxy, amino, C₁-C₆ alkyl, deuterated C₁-C₆ alkyl, C₁-C₆ hydroxyalkyl, C₁-C₆ haloalkyl, C₁-C₆ alkoxy, —NH(C₁-C₆ alkyl), —N(C₁-C₆ alkyl)₂, C₃-C₈ cycloalkyl, 4 to 10 membered heterocyclyl, C₆-C₁₀ aryl and 5 to 10 membered heteroaryl, wherein C₃-C₈ cycloalkyl, 4 to 10 membered heterocyclyl, C₆-C₁₀ aryl and 5 to 10 membered heteroaryl are each optionally further substituted by one or more hydrogen, deuterium, oxo, halogen, hydroxy, amino, nitro, cyano, ester group, carboxyl, C₁-C₆ alkyl, C₁-C₆ hydroxyalkyl, C₁-C₆ haloalkyl and C₁-C₆ alkoxy, —S(O)₂R₃₁ and —C(O)R₃₁.

R₃₁ is independently selected from hydrogen, halogen, hydroxy, amino, C₁-C₆ alkyl, deuterated C₁-C₆ alkyl, C₁-C₆ hydroxyalkyl, C₁-C₆ haloalkyl, C₁-C₆ alkoxy, —NH(C₁-C₆ alkyl) and —N(C₁-C₆ alkyl)₂.

m is an integer of 0, 1, 2, 3, 4 or 5.

In a more preferred embodiment, in the compound of formula (VII), the stereoisomer or the pharmaceutically acceptable salt, solvates and prodrugs thereof, each

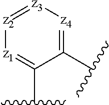

is independently selected from

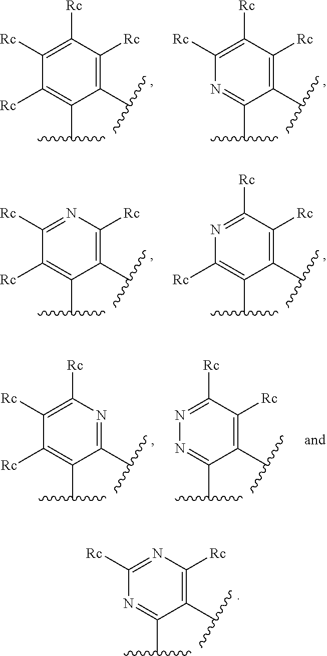

In a more preferred embodiment, in the compound of formula (VII), the stereoisomer or the pharmaceutically acceptable salt, solvates and prodrugs thereof, each Re is independently selected from hydrogen, halogen, hydroxy, cyano, C₁-C₄ alkyl, C₁-C₄ haloalkyl and C₁-C₄ alkoxy.

In a more preferred embodiment, in the compound of formula (VII), the stereoisomer or the pharmaceutically acceptable salt, solvates and prodrugs thereof, each Rₐ is independently selected from hydrogen, halogen, hydroxy, cyano. C₁-C₄ alkyl (such as methyl, ethyl), C₁₋C₁ haloalkyl (such as trifluoromethyl), C₁-C₄ hydroxyalkyl (such as hydroxymethyl), C₁-C₄ alkoxy (such as methoxy) and C₃-C₆ cycloalkyl (such as cyclopropyl).

In a more preferred embodiment, in the compound of formula (VII), the stereoisomer or the pharmaceutically acceptable salt, solvates and prodrugs thereof, each R₁ is independently selected from hydrogen, halogen, hydroxy, cyano, C₁-C₄ alkyl (such as methyl, ethyl), deuterated C₁-C₄ alkyl (such as methyl-d₃), C₁-C₄ haloalkyl (such as trifluoromethyl, difluoromethyl) and C₁-C₄ alkoxy and C₃-C₆ cycloalkyl (such as cyclopropyl).

In a more preferred embodiment, in the compound of formula (VII), the stereoisomer or the pharmaceutically acceptable salt, solvates and prodrugs thereof, each L₂ is bond.

In a more preferred embodiment, in the compound of formula (VII), the stereoisomer or the pharmaceutically acceptable salt, solvates and prodrugs thereof each ring B is independently selected from

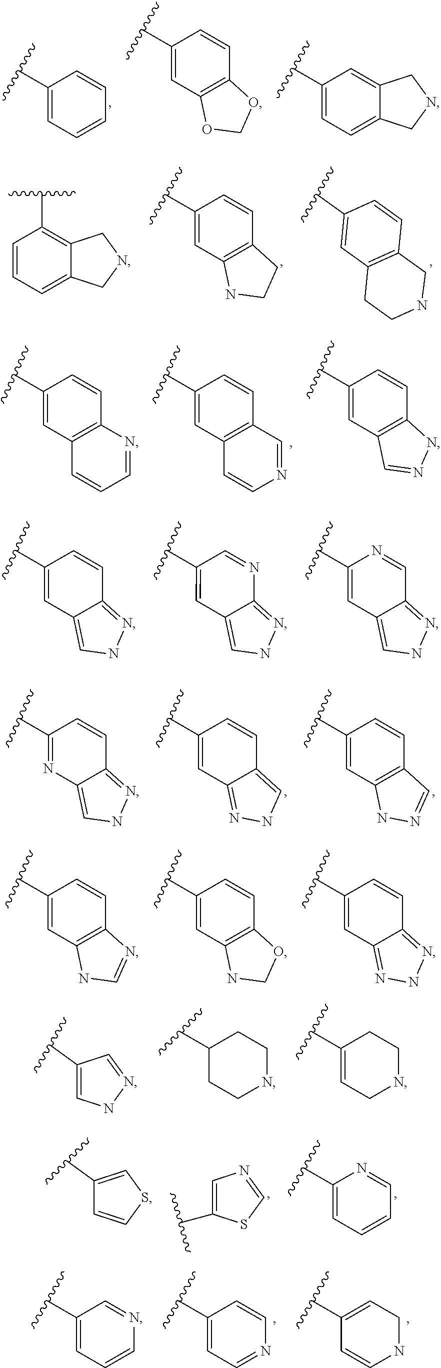

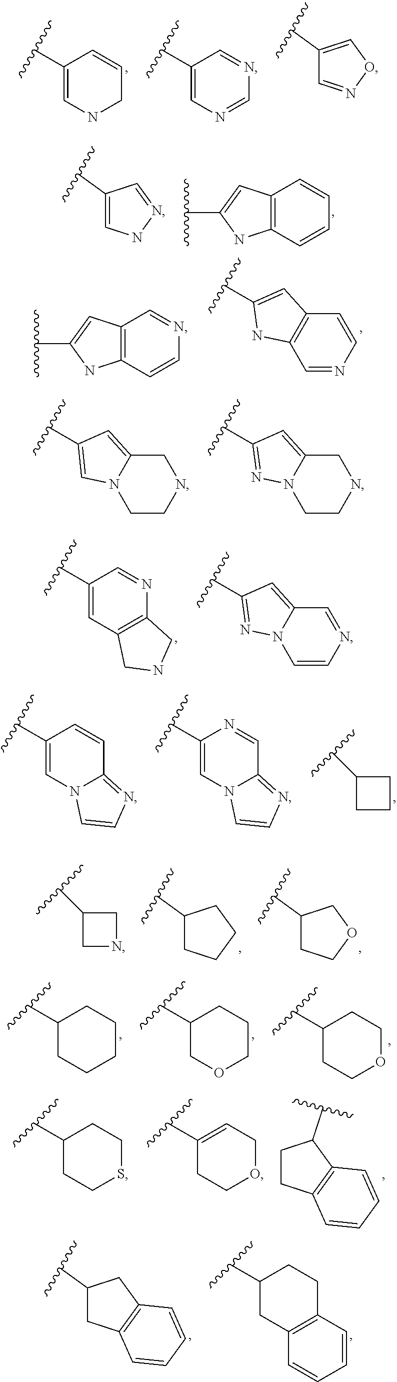

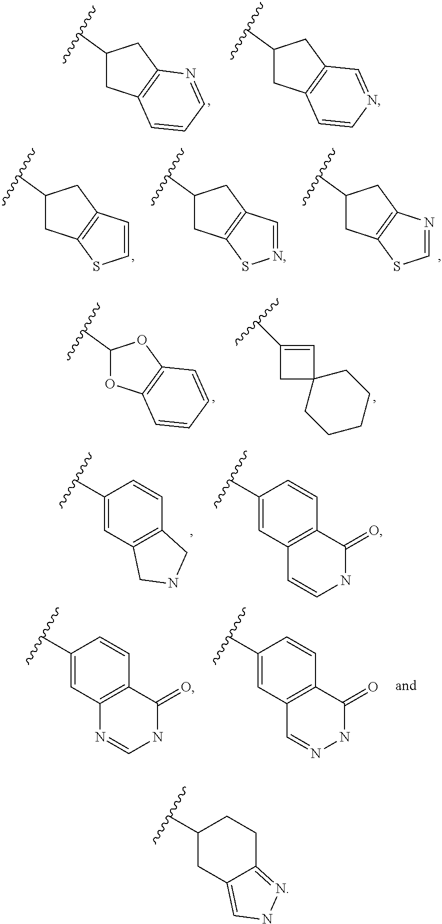

In a more preferred embodiment, in the compound of formula. (VII), the stereoisomer or the pharmaceutically acceptable salt, solvates and prodrugs thereof, each R₂ is independently selected from hydrogen, halogen, oxo, cyano, C₁-C₄alkyl (such as methyl, ethyl, propyl and isopropyl), C₁-C₄ haloalkyl (such as trifluoromethyl, 2-fluoro-2-methylpropyl, 1,1,1-tri fluoropropan-2-yl), C₁-C₄ hydroxyalkyl (such as 2-hydroxy-2-meth yl propyl) and C₃-C₆ cycloalkyl (such as cyclopropyl).

In a more preferred embodiment, in the compound of formula (VII), the stereoisomer or the pharmaceutically acceptable salt, solvates and prodrugs thereof, each L₃ is independently selected from bond, —O—, —CH₂—, —(CH₂)₂—, —C(O)—, —C(O)N(H)—, —C(O)N(C₁-C₄ alkyl)₂, —S(O)₂—, —CH₂—C(O)—, —CH(C₁-C₄ alkyl)-, —O—(CH₂)₁₋₄

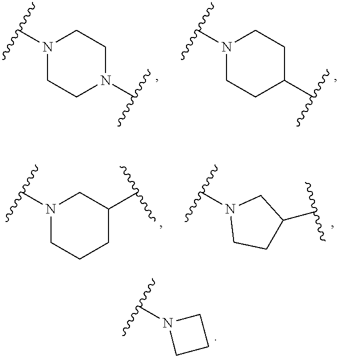 [SMILES: *N1CCN(*)CC1 ; *C1CCN(*)CC1 ; *C1CCCN(*)C1 ; *C1CCN(*)C1 ; *N1CCC1]

In a more preferred embodiment, in the compound of formula (VII), the stereoisomer or the pharmaceutically acceptable salt, solvates and prodrugs thereof, each R₃ is independently selected from hydrogen, —N(C₁-C₆ alkyl)₂,

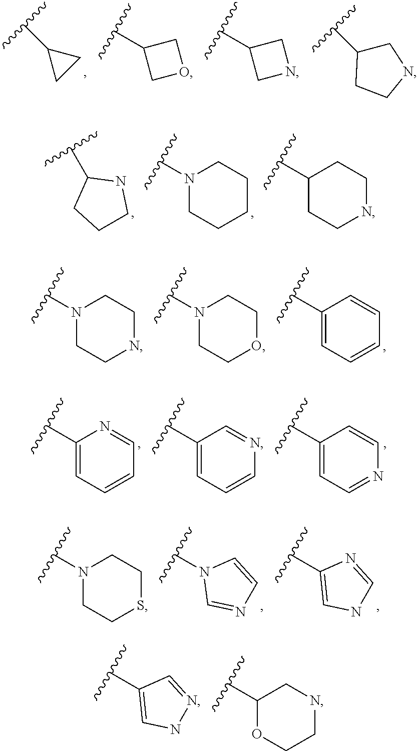

wherein each R₃ is optionally further substituted by one or more hydrogen, deuterium, oxo, halogen, hydroxy, amino, nitro, cyano, ester group, carboxyl, C₁-C₆ alkyl, C₁-C₆ hydroxyalkyl, C₁-C₆ haloalkyl and C₁-C₆ alkoxy, —S(O)₂R₃₁ and —C(O)R₃₁.

In a preferred embodiment of the present invention, the compound of formula (VII) is a compound of formula (VII-1), a stereoisomer thereof, or a pharmaceutically acceptable salt, solvates and prodrugs thereof:

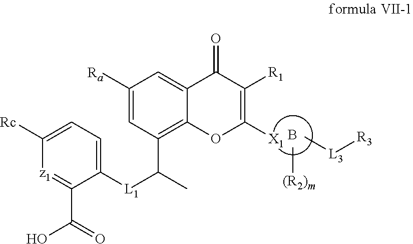

Wherein

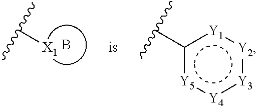

each Y₁, Y₂, Y₃, Y₄, and Y₅ is independently selected from C, CR_{y} and N;
R_{y} is hydrogen or two R_{y} and the atoms to which they are attached can form a 5 to 6 membered heterocycle or heteroaryl;
L₁, L₃, Rₐ, R_{c}, R₁, R₂, R₃, Z₁ and m are defined as in formula (VII).

In a more preferred embodiment, in the compound of formula (VII-1), the stereoisomer or the pharmaceutically acceptable salt, solvates and prodrugs thereof,

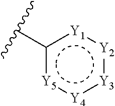

is selected from

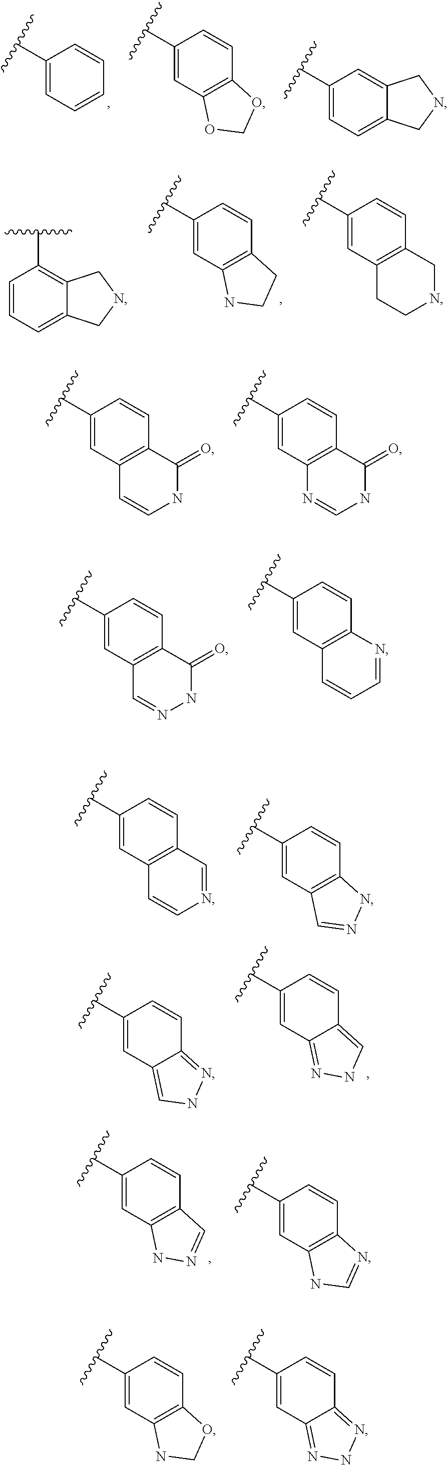

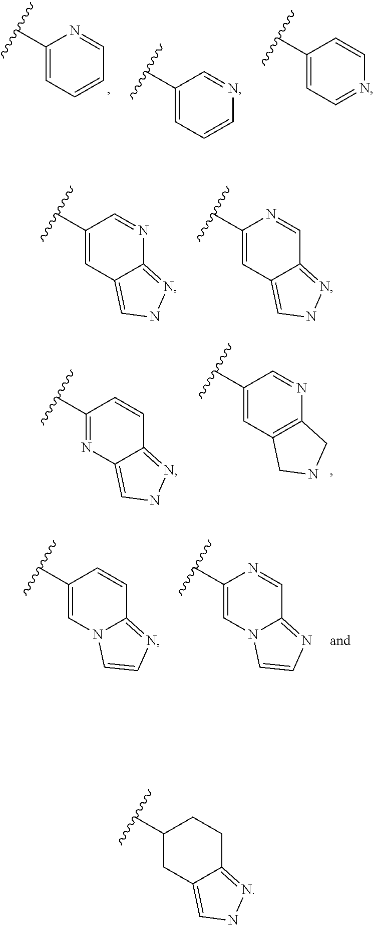

In another embodiment, described here are compounds of formula (VIII), a stereoisomer thereof, or a pharmaceutically acceptable salt, solvates and prodrugs thereof.

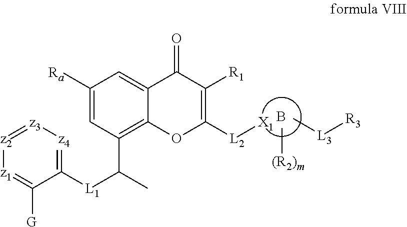

Wherein
G is selected from —C(O) R_{g}—C(O)OR_{g}, —OC(O)R_{g}, —C(O)N(R_{g})₂, —C(O)NH(O R_{g}), N(R_{g})C(O)R_{g}, —(O)₂R_{g}, —S(O)₂OR_{g}, —OS(O)₂R_{g}, —S(O)₂N(R_{g})₂, —N((R_{g})S(O)₂R_{g}, —P(O)(R_{g})₂, P(O)(OR_{g})₂, —OP(O)OR_{g}, —B(OR_{g})₂, —C(O)N(R_{g})—S(O)₂R_{g} and —S(O)₂N(R_{g})—C(O)R_{g}, C₃-C₈ cycloalkyl, 4 to 10 membered heterocyclyl, C₆-C₁₀ aryl and 5 to 10 membered heteroaryl, wherein C₃-C₅ cycloalkyl, 4 to 10 membered heterocyclyl, C₆-C₁₀ aryl and 5 to 10 membered heteroaryl are each optionally further substituted by one or more hydrogen, deuterium, oxo, halogen, hydroxy, amino, nitro, cyano, ester group, carboxyl, C₁-C₆ alkyl, C₁-C₆ hydroxyalkyl, C₁-C₆ haloalkyl and C₁-C₆ alkoxy.

Each R_{g} is independently selected from H, C₁-C₆ alkyl, C₂-C₆ alkenyl, C₂-C₆ alkynyl, C₁-C₆ haloalkyl, C₁-C₆ alkoxy, C₃-C₈ cycloalkyl, 4 to 10 membered heterocyclyl, C₆-C₁ aryl and 5 to 10 membered heteroaryl, wherein C₃-C₈ cycloalkyl, 4 to 10 membered heterocyclyl, C₆-C₁₀ aryl and 5 to 10 membered heteroaryl are each optionally further substituted by one or more hydrogen, deuterium, oxo, halogen, hydroxy, amino, nitro, cyano, ester group, carboxyl, C₁-C₆ alkyl, C₁-C₆ hydroxyalkyl, C₁-C₆ haloalkyl and C₁-C₆ alkoxy.

Z₁, Z₂, Z₃, Z₄, L₁, L₂, L₃, Rₐ, R₁, R₂, R₃, X₁, Ring B and m are defined as in formula (VII).

What's more, if needed, the preferred variables in the preferred embodiments of the compound of formula (VII), the stereoisomer or the pharmaceutically acceptable salt, solvates and prodrugs thereof, such as Z₁, Z₂, Z₃, Z₄, L₁, L₂, L₃, Rₐ, R₁, R₂, R₃, X₁, Ring B and m can be used for formula (VII-1) and formula (VIII).

In the most preferred embodiment, the compound of formula, (VI), (VII), (VII-1) or (VIII) the stereoisomer or the pharmaceutically acceptable salt, solvates and prodrugs thereof is selected from the table 1:

TABLE 1
Representative Compounds of formula (VI), (VII), (VII-1) or (VIII).

| # | Structure | Name |
|---|---|---|
| 1 | 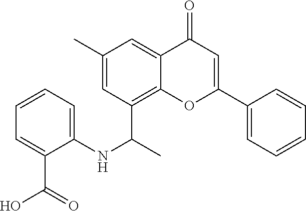 [SMILES: Cc1cc(C(C)Nc2ccccc2C(=O)O)c2oc(-c3ccccc3)cc(=O)c2c1] | 2-((1-(6-methyl-4-oxo-2- phenyl-4H-chromen-8- yl)ethyl)amino)benzoic acid |
| 2 | 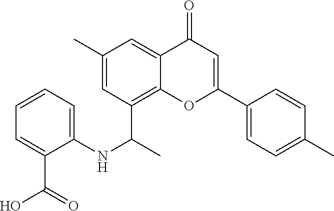 [SMILES: Cc1ccc(-c2cc(=O)c3cc(C)cc(C(C)Nc4ccccc4C(=O)O)c3o2)cc1] | 2-((1-(6-methyl-4-oxo-2- (p-tolyl)-4H-chromen-8- yl)ethyl)amino)benzoic acid |
| 3 | 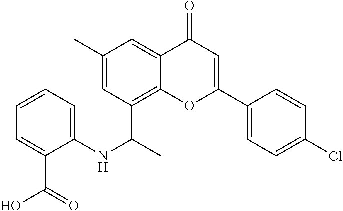 [SMILES: Cc1cc(C(C)Nc2ccccc2C(=O)O)c2oc(-c3ccc(Cl)cc3)cc(=O)c2c1] | 2-((1-(2-(4-chlorophenyl)-6- methyl-4-oxo-4H-chromen-8- yl)ethyl)amino)benzoic acid |
| 4 | 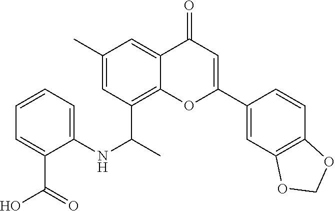 [SMILES: Cc1cc(C(C)Nc2ccccc2C(=O)O)c2oc(-c3ccc4c(c3)OCO4)cc(=O)c2c1] | 2-((1-(2-(benzo[d][1,3]dioxol- 5-yl)-6-methyl-4-oxo-4H- chromen-8-yl)ethyl)amino)benzoic acid |
| 5 | 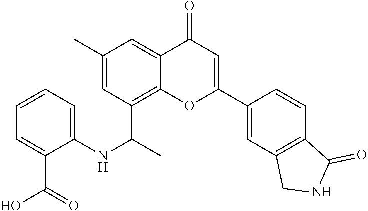 [SMILES: Cc1cc(C(C)Nc2ccccc2C(=O)O)c2oc(-c3ccc4c(c3)CNC4=O)cc(=O)c2c1] | 2-((1-(6-methyl-4-oxo-2-(1-oxoisoindolin- 5-yl)-4H-chromen-8- yl)ethyl)amino)benzoic acid |
| 6 | 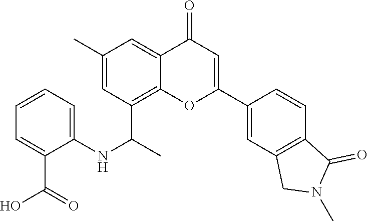 [SMILES: Cc1cc(C(C)Nc2ccccc2C(=O)O)c2oc(-c3ccc4c(c3)CN(C)C4=O)cc(=O)c2c1] | 2-((1-(6-methyl-2-(2-methyl-1- oxoisoindolin-5-yl)-4-oxo-4H- chromen-8-yl)ethyl)amino)benzoic acid |
| 7 | 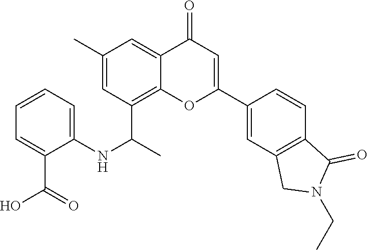 [SMILES: CCN1Cc2cc(-c3cc(=O)c4cc(C)cc(C(C)Nc5ccccc5C(=O)O)c4o3)ccc2C1=O] | 2-((1-(2-(2-ethyl-1-oxoisoindolin- 5-yl)-6-methyl-4-oxo-4H- chromen-8-yl)ethyl)amino)benzoic acid |
| 8 | 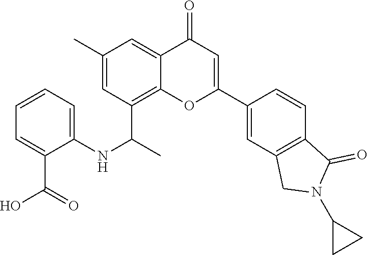 [SMILES: Cc1cc(C(C)Nc2ccccc2C(=O)O)c2oc(-c3ccc4c(c3)CN(C3CC3)C4=O)cc(=O)c2c1] | 2-((1-(2-(2-cyclopropyl-1-oxoisoindolin- 5-yl)-6-methyl-4-oxo- 4H-chromen-8-yl)ethyl)amino)benzoic acid |
| 9 | 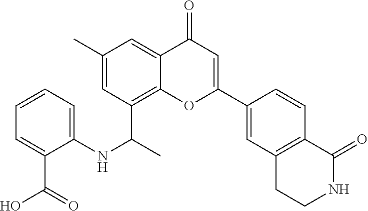 [SMILES: Cc1cc(C(C)Nc2ccccc2C(=O)O)c2oc(-c3ccc4c(c3)CCNC4=O)cc(=O)c2c1] | 2-((1-(6-methyl-4-oxo-2-(1-oxo- 1,2,3,4-tetrahydroisoquinolin- 6-yl)-4H-chromen-8-yl)ethyl)amino)benzoic acid |
| 10 | 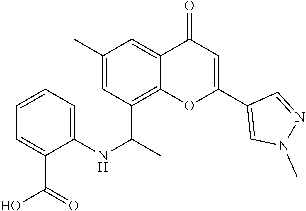 [SMILES: Cc1cc(C(C)Nc2ccccc2C(=O)O)c2oc(-c3cnn(C)c3)cc(=O)c2c1] | 2-((1-(6-methyl-2-(1-methyl- 1H-pyrazol-4-yl)-4-oxo-4H- chromen-8-yl)ethyl)amino)benzoic acid |
| 11 | 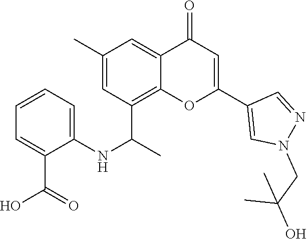 [SMILES: Cc1cc(C(C)Nc2ccccc2C(=O)O)c2oc(-c3cnn(CC(C)(C)O)c3)cc(=O)c2c1] | 2-((1-(2-(1-(2-hydroxy-2-methylpropyl)- 1H-pyrazol-4-yl)-6- methyl-4-oxo-4H-chromen-8-yl) ethyl)amino)benzoic acid |
| 12 | 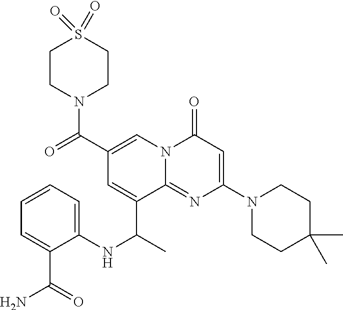 [SMILES: CC(Nc1ccccc1C(N)=O)c1cc(C(=O)N2CCS(=O)(=O)CC2)cn2c(=O)cc(N3CCC(C)(C)CC3)nc12] | 2-((1-(2-(4,4-dimethylpiperidin- 1-yl)-7-(1,1- dioxidothiomorpholine-4-carbonyl)- 4-oxo-4H-pyrido[1,2- a]pyrimidin-9-yl)ethyl)amino)benzamide |
| 13 | 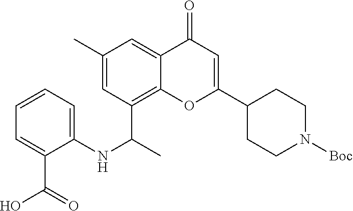 [SMILES: Cc1cc(C(C)Nc2ccccc2C(=O)O)c2oc(C3CCN(C(=O)OC(C)(C)C)CC3)cc(=O)c2c1] | 2-((1-(2-(1-(tert-butoxycarbonyl) piperidin-4-yl)-6-methyl-4- oxo-4H-chromen-8-yl) ethyl)amino)benzoic acid |
| 14 | 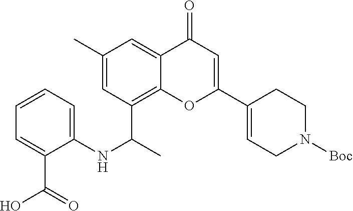 [SMILES: Cc1cc(C(C)Nc2ccccc2C(=O)O)c2oc(C3=CCN(C(=O)OC(C)(C)C)CC3)cc(=O)c2c1] | 2-((1-(2-(1-(tert-butoxycarbonyl)- 1,2,3,6-tetrahydropyridin-4- yl)-6-methyl-4-oxo-4H-chromen- 8-yl)ethyl)amino)benzoic acid |
| 15 | 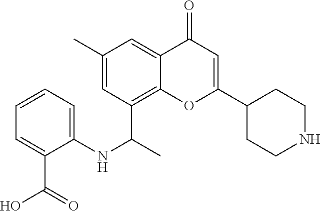 [SMILES: Cc1cc(C(C)Nc2ccccc2C(=O)O)c2oc(C3CCNCC3)cc(=O)c2c1] | 2-((1-(6-methyl-4-oxo-2-(piperidin- 4-yl)-4H-chromen-8- yl)ethyl)amino)benzoic acid |
| 16 | 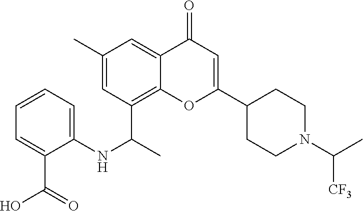 [SMILES: Cc1cc(C(C)Nc2ccccc2C(=O)O)c2oc(C3CCN(C(C)C(F)(F)F)CC3)cc(=O)c2c1] | 2-((1-(6-methyl-4-oxo-2- (1-(1,1,1-trifluoropropan-2- yl)piperidin-4-yl)-4H-chromen- 8-yl)ethyl)amino)benzoic acid |
| 17 | 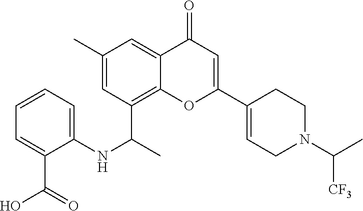 [SMILES: Cc1cc(C(C)Nc2ccccc2C(=O)O)c2oc(C3=CCN(C(C)C(F)(F)F)CC3)cc(=O)c2c1] | 2-((1-(6-methyl-4-oxo-2-(1- (1,1,1-trifluoropropan-2-yl)- 1,2,3,6-tetrahydropyridin- 4-yl)-4H-chromen-8- yl)ethyl)amino)benzoic acid |
| 18 | 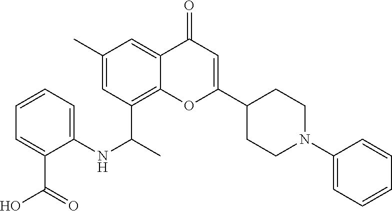 [SMILES: Cc1cc(C(C)Nc2ccccc2C(=O)O)c2oc(C3CCN(c4ccccc4)CC3)cc(=O)c2c1] | 2-((1-(6-methyl-4-oxo-2-(1- phenylpiperidin-4-yl)-4H-chromen- 8-yl)ethyl)amino)benzoic acid |
| 19 | 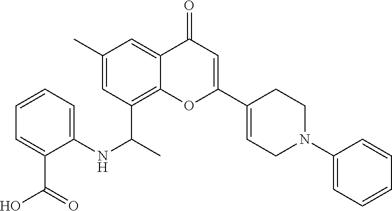 [SMILES: Cc1cc(C(C)Nc2ccccc2C(=O)O)c2oc(C3=CCN(c4ccccc4)CC3)cc(=O)c2c1] | 2-((1-(6-methyl-4-oxo-2-(1-phenyl- 1,2,3,6-tetrahydropyridin-4- yl)-4H-chromen-8-yl)ethyl) amino)benzoic acid |
| 20 | 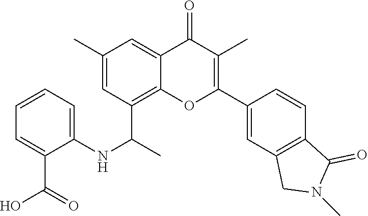 [SMILES: Cc1cc(C(C)Nc2ccccc2C(=O)O)c2oc(-c3ccc4c(c3)CN(C)C4=O)c(C)c(=O)c2c1] | 2-((1-(3,6-dimethyl-2-(2-methyl-1- oxoisoindolin-5-yl)-4-oxo- 4H-chromen-8-yl)ethyl) amino)benzoic acid |
| 21 | 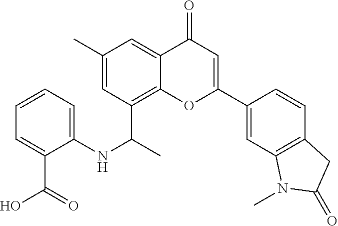 [SMILES: Cc1cc(C(C)Nc2ccccc2C(=O)O)c2oc(-c3ccc4c(c3)N(C)C(=O)C4)cc(=O)c2c1] | 2-((1-(6-methyl-2-(1-methyl-2- oxoindolin-6-yl)-4-oxo-4H- chromen-8-yl)ethyl) amino)benzoic acid |
| 22 | 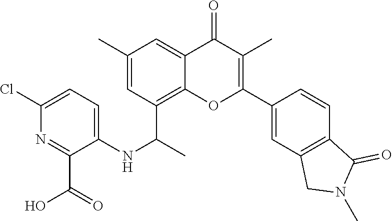 [SMILES: Cc1cc(C(C)Nc2ccc(Cl)nc2C(=O)O)c2oc(-c3ccc4c(c3)CN(C)C4=O)c(C)c(=O)c2c1] | 6-chloro-3-((1-(3,6-dimethyl-2- (2-methyl-1-oxoisoindolin-5- yl)-4-oxo-4H-chromen-8-yl) ethyl)amino)picolinic acid |
| 23 | 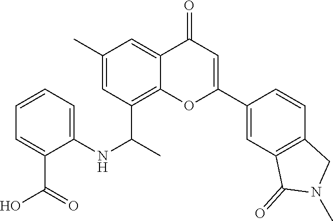 [SMILES: Cc1cc(C(C)Nc2ccccc2C(=O)O)c2oc(-c3ccc4c(c3)C(=O)N(C)C4)cc(=O)c2c1] | 2-((1-(6-methyl-2-(2-methyl-3- oxoisoindolin-5-yl)-4-oxo-4H- chromen-8-yl)ethyl) amino)benzoic acid |
| 24 | 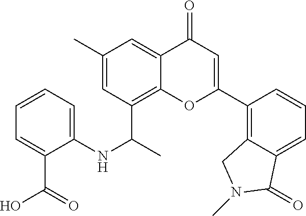 [SMILES: Cc1cc(C(C)Nc2ccccc2C(=O)O)c2oc(-c3cccc4c3CN(C)C4=O)cc(=O)c2c1] | 2-((1-(6-methyl-2-(2-methyl-1- oxoisoindolin-4-yl)-4-oxo-4H- chromen-8-yl)ethyl)amino)benzoic acid |
| 25 | 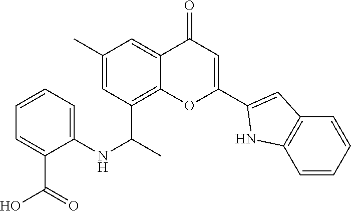 [SMILES: Cc1cc(C(C)Nc2ccccc2C(=O)O)c2oc(-c3cc4ccccc4[nH]3)cc(=O)c2c1] | 2-((1-(2-(1H-indol-2-yl)-6- methyl-4-oxo-4H-chromen-8- yl)ethyl)amino)benzoic acid |
| 26 | 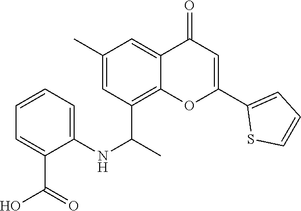 [SMILES: Cc1cc(C(C)Nc2ccccc2C(=O)O)c2oc(-c3cccs3)cc(=O)c2c1] | 2-((1-(6-methyl-4-oxo-2- (thiophen-2-yl)-4H-chromen-8- yl)ethyl)amino)benzoic acid |
| 27 | 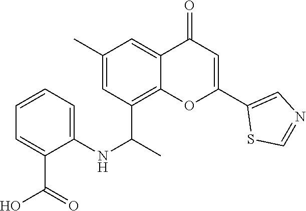 [SMILES: Cc1cc(C(C)Nc2ccccc2C(=O)O)c2oc(-c3cncs3)cc(=O)c2c1] | 2-((1-(6-methyl-4-oxo-2- (thiazol-5-yl)-4H-chromen-8- yl)ethyl)amino)benzoic acid |
| 28 | 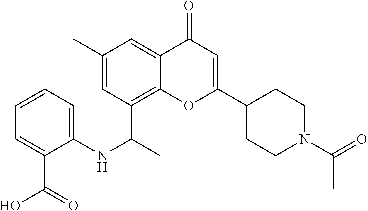 [SMILES: CC(=O)N1CCC(c2cc(=O)c3cc(C)cc(C(C)Nc4ccccc4C(=O)O)c3o2)CC1] | 2-((1-(2-(1-acetylpiperidin-4-yl)- 6-methyl-4-oxo-4H-chromen- 8-yl)ethyl)amino)benzoic acid |
| 29 | 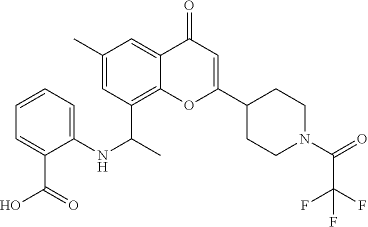 [SMILES: Cc1cc(C(C)Nc2ccccc2C(=O)O)c2oc(C3CCN(C(=O)C(F)(F)F)CC3)cc(=O)c2c1] | 2-((1-(6-methyl-4-oxo-2-(1- (2,2,2-trifluoroacetyl)piperidin-4- yl)-4H-chromen-8-yl)ethyl) amino)benzoic acid |
| 30 | 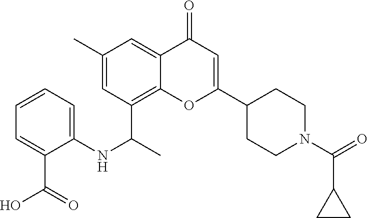 [SMILES: Cc1cc(C(C)Nc2ccccc2C(=O)O)c2oc(C3CCN(C(=O)C4CC4)CC3)cc(=O)c2c1] | 2-((1-(2-(1-(cyclopropanecarbonyl) piperidin-4-yl)-6-methyl-4- oxo-4H-chromen-8-yl)ethyl) amino)benzoic acid |
| 31 | 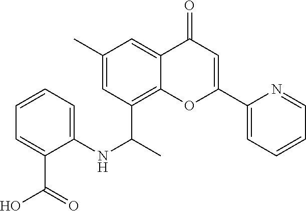 [SMILES: Cc1cc(C(C)Nc2ccccc2C(=O)O)c2oc(-c3ccccn3)cc(=O)c2c1] | 2-((1-(6-methyl-4-oxo-2- (pyridin-2-yl)-4H-chromen-8- yl)ethyl)amino)benzoic acid |
| 32 | 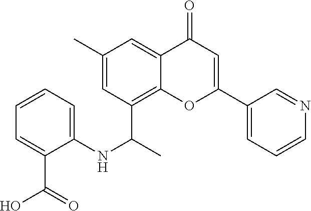 [SMILES: Cc1cc(C(C)Nc2ccccc2C(=O)O)c2oc(-c3cccnc3)cc(=O)c2c1] | 2-((1-(6-methyl-4-oxo-2- (pyridin-3-yl)-4H-chromen-8- yl)ethyl)amino)benzoic acid |
| 33 | 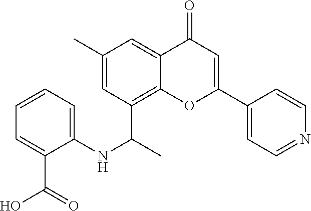 [SMILES: Cc1cc(C(C)Nc2ccccc2C(=O)O)c2oc(-c3ccncc3)cc(=O)c2c1] | 2-((1-(6-methyl-4-oxo-2- (pyridin-4-yl)-4H-chromen-8- yl)ethyl)amino)benzoic acid |
| 34 | 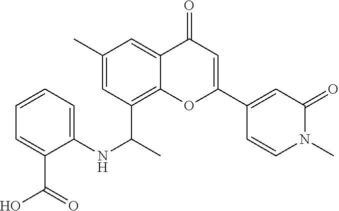 [SMILES: Cc1cc(C(C)Nc2ccccc2C(=O)O)c2oc(-c3ccn(C)c(=O)c3)cc(=O)c2c1] | 2-((1-(6-methyl-2-(1-methyl-2-oxo- 1,2-dihydropyridin-4-yl)-4- oxo-4H-chromen-8-yl)ethyl) amino)benzoic acid |
| 35 | 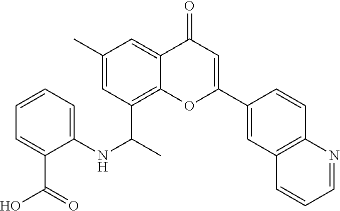 [SMILES: Cc1cc(C(C)Nc2ccccc2C(=O)O)c2oc(-c3ccc4ncccc4c3)cc(=O)c2c1] | 2-((1-(6-methyl-4-oxo-2- (quinolin-6-yl)-4H-chromen-8- yl)ethyl)amino)benzoic acid |
| 36 | 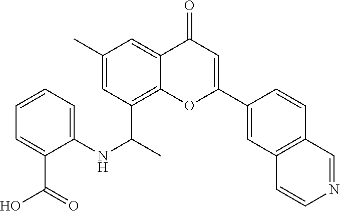 [SMILES: Cc1cc(C(C)Nc2ccccc2C(=O)O)c2oc(-c3ccc4cnccc4c3)cc(=O)c2c1] | 2-((1-(2-(isoquinolin-6-yl)-6- methyl-4-oxo-4H-chromen-8- yl)ethyl)amino)benzoic acid |
| 37 | 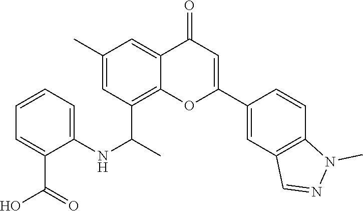 [SMILES: Cc1cc(C(C)Nc2ccccc2C(=O)O)c2oc(-c3ccc4c(cnn4C)c3)cc(=O)c2c1] | 2-((1-(6-methyl-2-(1-methyl- 1H-indazol-5-yl)-4-oxo-4H- chromen-8-yl)ethyl)amino)benzoic acid |
| 38 | 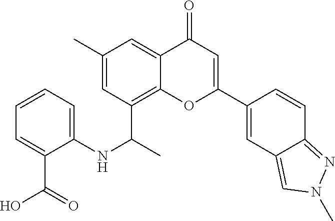 [SMILES: Cc1cc(C(C)Nc2ccccc2C(=O)O)c2oc(-c3ccc4nn(C)cc4c3)cc(=O)c2c1] | 2-((1-(6-methyl-2-(2-methyl-2H- indazol-5-yl)-4-oxo-4H- chromen-8-yl)ethyl)amino)benzoic acid |
| 39 | 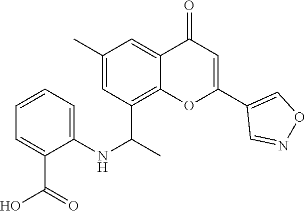 [SMILES: Cc1cc(C(C)Nc2ccccc2C(=O)O)c2oc(-c3cnoc3)cc(=O)c2c1] | 2-((1-(2-(isoxazol-4-yl)-6- methyl-4-oxo-4H-chromen-8- yl)ethyl)amino)benzoic acid |
| 40 | 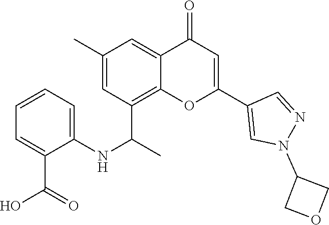 [SMILES: Cc1cc(C(C)Nc2ccccc2C(=O)O)c2oc(-c3cnn(C4COC4)c3)cc(=O)c2c1] | 2-((1-(6-methyl-2-(1-(oxetan-3- yl)-1H-pyrazol-4-yl)-4-oxo-4H- chromen-8-yl)ethyl) amino)benzoic acid |
| 41 | 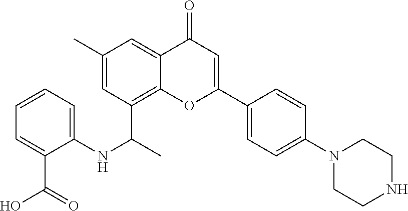 [SMILES: Cc1cc(C(C)Nc2ccccc2C(=O)O)c2oc(-c3ccc(N4CCNCC4)cc3)cc(=O)c2c1] | 2-((1-(6-methyl-4-oxo-2-(4- (piperazin-1-yl)phenyl)-4H- chromen-8-yl)ethyl) amino)benzoic acid |
| 42 | 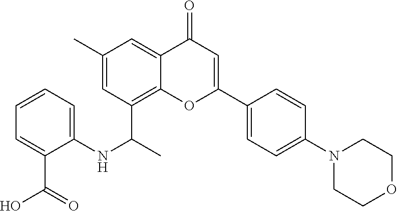 [SMILES: Cc1cc(C(C)Nc2ccccc2C(=O)O)c2oc(-c3ccc(N4CCOCC4)cc3)cc(=O)c2c1] | 2-((1-(6-methyl-2-(4-morpholinophenyl)- 4-oxo-4H-chromen-8- yl)ethyl)amino)benzoic acid |
| 43 | 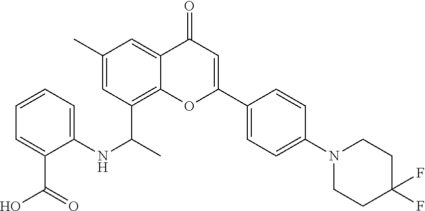 [SMILES: Cc1cc(C(C)Nc2ccccc2C(=O)O)c2oc(-c3ccc(N4CCC(F)(F)CC4)cc3)cc(=O)c2c1] | 2-((1-(2-(4-(4,4-difluoropiperidin- 1-yl)phenyl)-6-methyl-4- oxo-4H-chromen-8-yl)ethyl) amino)benzoic acid |
| 44 | 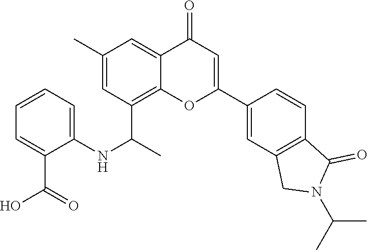 [SMILES: Cc1cc(C(C)Nc2ccccc2C(=O)O)c2oc(-c3ccc4c(c3)CN(C(C)C)C4=O)cc(=O)c2c1] | 2-((1-(2-(2-isopropyl-1-oxoisoindolin- 5-yl)-6-methyl-4-oxo- 4H-chromen-8-yl)ethyl) amino)benzoic acid |
| 45 | 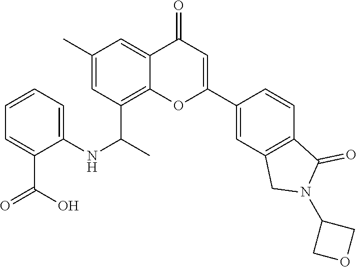 [SMILES: Cc1cc(C(C)Nc2ccccc2C(=O)O)c2oc(-c3ccc4c(c3)CN(C3COC3)C4=O)cc(=O)c2c1] | 2-((1-(6-methyl-2-(2-(oxetan-3-yl)- 1-oxoisoindolin-5-yl)-4- oxo-4H-chromen-8-yl)ethyl) amino)benzoic acid |
| 46 | 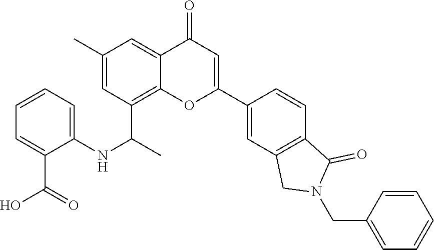 [SMILES: Cc1cc(C(C)Nc2ccccc2C(=O)O)c2oc(-c3ccc4c(c3)CN(Cc3ccccc3)C4=O)cc(=O)c2c1] | 2-((1-(2-(2-benzyl-1-oxoisoindolin-5- yl)-6-methyl-4-oxo-4H- chromen-8-yl)ethyl)amino)benzoic acid |
| 47 | 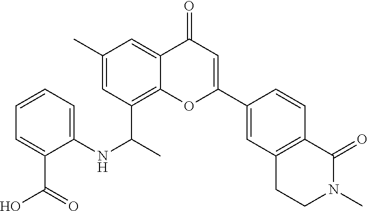 [SMILES: Cc1cc(C(C)Nc2ccccc2C(=O)O)c2oc(-c3ccc4c(c3)CCN(C)C4=O)cc(=O)c2c1] | 2-((1-(6-methyl-2-(2-methyl-1-oxo-1,2,3,4- tetrahydroisoquinolin-6-yl)- 4-oxo-4H-chromen-8- yl)ethyl)amino)benzoic acid |
| 48 | 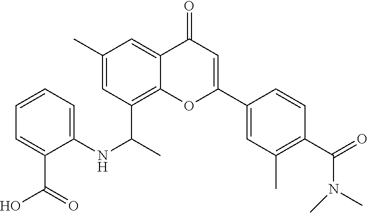 [SMILES: Cc1cc(C(C)Nc2ccccc2C(=O)O)c2oc(-c3ccc(C(=O)N(C)C)c(C)c3)cc(=O)c2c1] | 2-((1-(2-(4-(dimethylcarbamoyl)-3- methylphenyl)-6-methyl-4- oxo-4H-chromen-8-yl)ethyl) amino)benzoic acid |
| 49 | 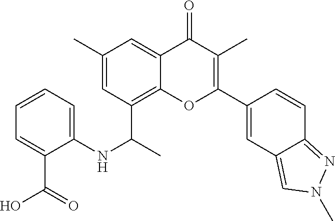 [SMILES: Cc1cc(C(C)Nc2ccccc2C(=O)O)c2oc(-c3ccc4nn(C)cc4c3)c(C)c(=O)c2c1] | 2-((1-(3,6-dimethyl-2-(2-methyl- 2H-indazol-5-yl)-4-oxo-4H- chromen-8-yl)ethyl) amino)benzoic acid |
| 50 | 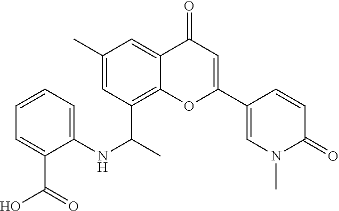 [SMILES: Cc1cc(C(C)Nc2ccccc2C(=O)O)c2oc(-c3ccc(=O)n(C)c3)cc(=O)c2c1] | 2-((1-(6-methyl-2-(1-methyl-6-oxo- 1,6-dihydropyridin-3-yl)-4- oxo-4H-chromen-8-yl)ethyl) amino)benzoic acid |
| 51 | 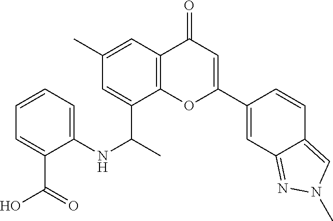 [SMILES: Cc1cc(C(C)Nc2ccccc2C(=O)O)c2oc(-c3ccc4cn(C)nc4c3)cc(=O)c2c1] | 2-((1-(6-methyl-2-(2-methyl-2H- indazol-6-yl)-4-oxo-4H- chromen-8-yl)ethyl) amino)benzoic acid |
| 52 | 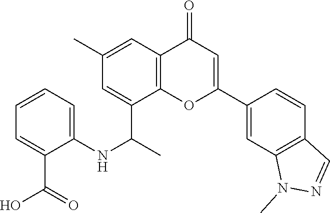 [SMILES: Cc1cc(C(C)Nc2ccccc2C(=O)O)c2oc(-c3ccc4cnn(C)c4c3)cc(=O)c2c1] | 2-((1-(6-methyl-2-(1-methyl- 1H-indazol-6-yl)-4-oxo-4H- chromen-8-yl)ethyl) amino)benzoic acid |
| 53 | 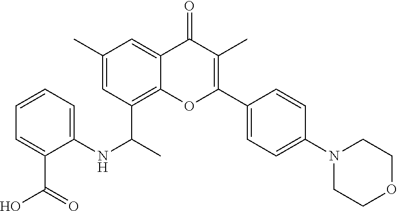 [SMILES: Cc1cc(C(C)Nc2ccccc2C(=O)O)c2oc(-c3ccc(N4CCOCC4)cc3)c(C)c(=O)c2c1] | 2-((1-(3,6-dimethyl-2-(4- morpholinophenyl)-4-oxo-4H- chromen-8-yl)ethyl) amino)benzoic acid |
| 54 | 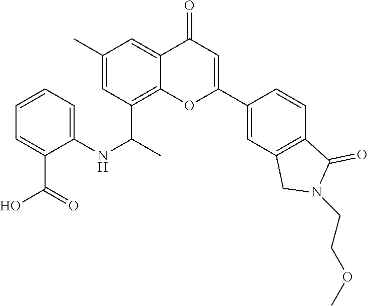 [SMILES: COCCN1Cc2cc(-c3cc(=O)c4cc(C)cc(C(C)Nc5ccccc5C(=O)O)c4o3)ccc2C1=O] | 2-((1-(2-(2-(2-methoxyethyl)- 1-oxoisoindolin-5-yl)-6-methyl- 4-oxo-4H-chromen-8-yl) ethyl)amino)benzoic acid |
| 55 | 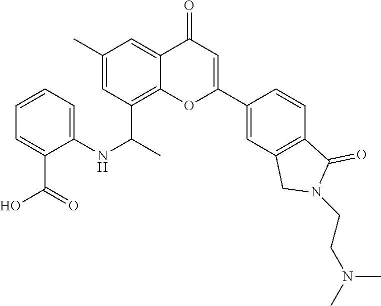 [SMILES: Cc1cc(C(C)Nc2ccccc2C(=O)O)c2oc(-c3ccc4c(c3)CN(CCN(C)C)C4=O)cc(=O)c2c1] | 2-((1-(2-(2-(2-(dimethylamino) ethyl)-1-oxoisoindolin-5-yl)-6- methyl-4-oxo-4H-chromen-8- yl)ethyl)amino)benzoic acid |
| 56 | 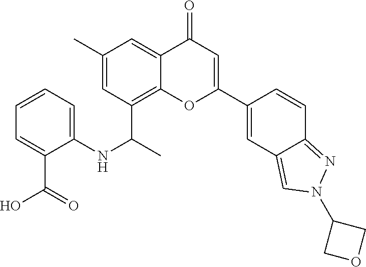 [SMILES: Cc1cc(C(C)Nc2ccccc2C(=O)O)c2oc(-c3ccc4nn(C5COC5)cc4c3)cc(=O)c2c1] | 2-((1-(6-methyl-2-(2-(oxetan-3-yl)- 2H-indazol-5-yl)-4-oxo-4H- chromen-8-yl)ethyl)amino)benzoic acid |
| 57 | 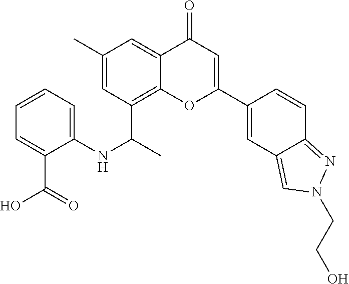 [SMILES: Cc1cc(C(C)Nc2ccccc2C(=O)O)c2oc(-c3ccc4nn(CCO)cc4c3)cc(=O)c2c1] | 2-((1-(2-(2-(2-hydroxyethyl)-2H- indazol-5-yl)-6-methyl-4-oxo- 4H-chromen-8-yl)ethyl)amino)benzoic acid |
| 58 | 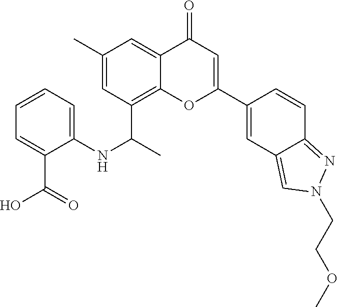 [SMILES: COCCn1cc2cc(-c3cc(=O)c4cc(C)cc(C(C)Nc5ccccc5C(=O)O)c4o3)ccc2n1] | 2-((1-(2-(2-(2-methoxyethyl)- 2H-indazol-5-yl)-6-methyl-4- oxo-4H-chromen-8-yl)ethyl) amino)benzoic acid |
| 59 | 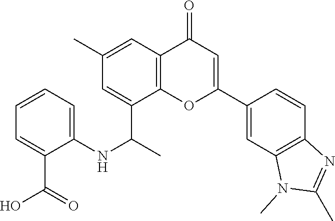 [SMILES: Cc1cc(C(C)Nc2ccccc2C(=O)O)c2oc(-c3ccc4nc(C)n(C)c4c3)cc(=O)c2c1] | 2-((1-(2-(1,2-dimethyl-1H-benzo [d]imidazol-6-yl)-6-methyl-4- oxo-4H-chromen-8-yl)ethyl) amino)benzoic acid |
| 60 | 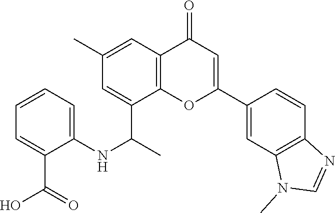 [SMILES: Cc1cc(C(C)Nc2ccccc2C(=O)O)c2oc(-c3ccc4ncn(C)c4c3)cc(=O)c2c1] | 2-((1-(6-methyl-2-(1-methyl-1H- benzo[d]imidazol-6-yl)-4-oxo- 4H-chromen-8-yl)ethyl) amino)benzoic acid |
| 61 | 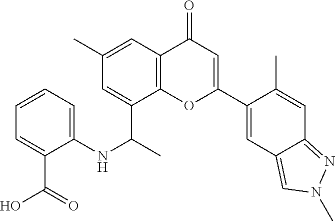 [SMILES: Cc1cc(C(C)Nc2ccccc2C(=O)O)c2oc(-c3cc4cn(C)nc4cc3C)cc(=O)c2c1] | 2-((1-(2-(2,6-dimethyl-2H-indazol- 5-yl)-6-methyl-4-oxo-4H- chromen-8-yl)ethyl) amino)benzoic acid |
| 62 | 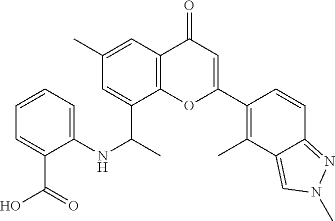 [SMILES: Cc1cc(C(C)Nc2ccccc2C(=O)O)c2oc(-c3ccc4nn(C)cc4c3C)cc(=O)c2c1] | 2-((1-(2-(2,4-dimethyl-2H-indazol- 5-yl)-6-methyl-4-oxo-4H- chromen-8-yl)ethyl) amino)benzoic acid |
| 63 | 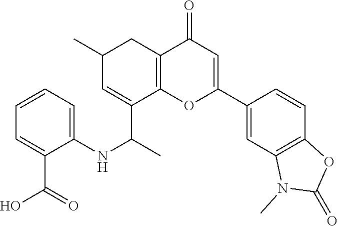 [SMILES: CC1C=C(C(C)Nc2ccccc2C(=O)O)c2oc(-c3ccc4oc(=O)n(C)c4c3)cc(=O)c2C1] | 2-((1-(6-methyl-2-(3-methyl- 2-oxo-2,3-dihydrobenzo[d]oxazol- 5-yl)-4-oxo-5,6-dihydro-4H- chromen-8-yl)ethyl)amino)benzoic acid |
| 64 | 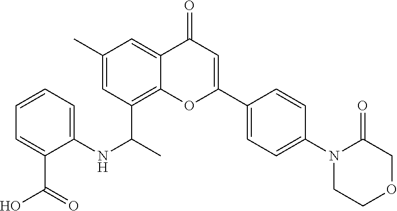 [SMILES: Cc1cc(C(C)Nc2ccccc2C(=O)O)c2oc(-c3ccc(N4CCOCC4=O)cc3)cc(=O)c2c1] | 2-((1-(6-methyl-4-oxo-2-(4-(3- oxomorpholino)phenyl)-4H- chromen-8-yl)ethyl) amino)benzoic acid |
| 65 | 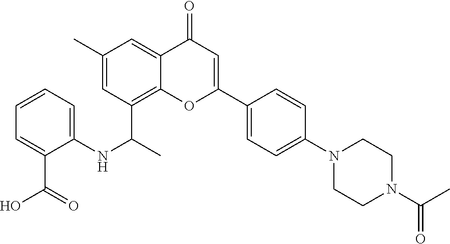 [SMILES: CC(=O)N1CCN(c2ccc(-c3cc(=O)c4cc(C)cc(C(C)Nc5ccccc5C(=O)O)c4o3)cc2)CC1] | 2-((1-(2-(4-(4-acetylpiperazin-1- yl)phenyl)-6-methyl-4-oxo- 4H-chromen-8-yl)ethyl) amino)benzoic acid |
| 66 | 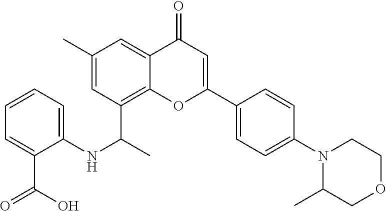 [SMILES: Cc1cc(C(C)Nc2ccccc2C(=O)O)c2oc(-c3ccc(N4CCOCC4C)cc3)cc(=O)c2c1] | 2-((1-(6-methyl-2-(4-(3-methylmorpholino) phenyl)-4-oxo-4H- chromen-8-yl)ethyl) amino)benzoic acid |
| 67 | 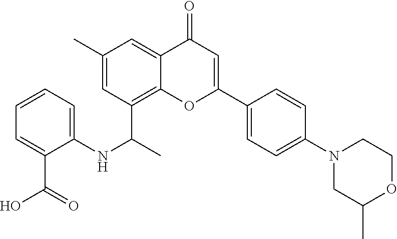 [SMILES: Cc1cc(C(C)Nc2ccccc2C(=O)O)c2oc(-c3ccc(N4CCOC(C)C4)cc3)cc(=O)c2c1] | 2-((1-(6-methyl-2-(4-(2- methylmorpholino)phenyl)-4-oxo-4H- chromen-8-yl)ethyl) amino)benzoic acid |
| 68 | 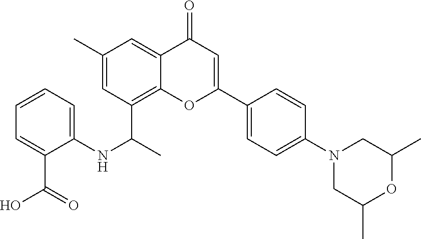 [SMILES: Cc1cc(C(C)Nc2ccccc2C(=O)O)c2oc(-c3ccc(N4CC(C)OC(C)C4)cc3)cc(=O)c2c1] | 2-((1-(2-(4-(2,6-dimethylmorpholino) phenyl)-6-methyl-4-oxo- 4H-chromen-8-yl)ethyl) amino)benzoic acid |
| 69 | 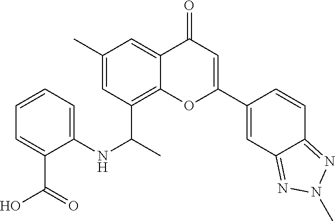 [SMILES: Cc1cc(C(C)Nc2ccccc2C(=O)O)c2oc(-c3ccc4nn(C)nc4c3)cc(=O)c2c1] | 2-((1-(6-methyl-2-(2-methyl-2H- benzo[d][1,2,3]triazol-5-yl)-4- oxo-4H-chromen-8-yl) ethyl)amino)benzoic acid |
| 70 | 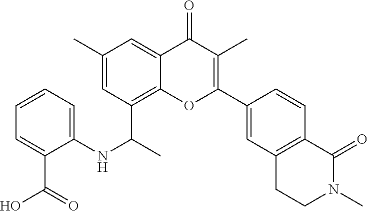 [SMILES: Cc1cc(C(C)Nc2ccccc2C(=O)O)c2oc(-c3ccc4c(c3)CCN(C)C4=O)c(C)c(=O)c2c1] | 2-((1-(3,6-dimethyl-2- (2-methyl-1-oxo-1,2,3,4- tetrahydroisoquinolin-6- yl)-4-oxo-4H-chromen-8- yl)ethyl)amino)benzoic acid |
| 71 | 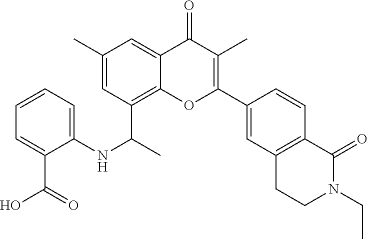 [SMILES: CCN1CCc2cc(-c3oc4c(C(C)Nc5ccccc5C(=O)O)cc(C)cc4c(=O)c3C)ccc2C1=O] | 2-((1-(2-(2-ethyl-1-oxo-1,2,3,4- tetrahydroisoquinolin-6-yl)-3,6- dimethyl-4-oxo-4H-chromen- 8-yl)ethyl)amino)benzoic acid |
| 72 | 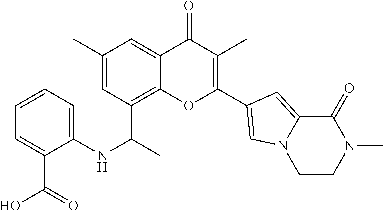 [SMILES: Cc1cc(C(C)Nc2ccccc2C(=O)O)c2oc(-c3cc4n(c3)CCN(C)C4=O)c(C)c(=O)c2c1] | 2-((1-(3,6-dimethyl-2- (2-methyl-1-oxo-1,2,3,4- tetrahydropyrrolo[1,2-a]pyrazin- 7-yl)-4-oxo-4H-chromen-8- yl)ethyl)amino)benzoic acid |
| 73 | 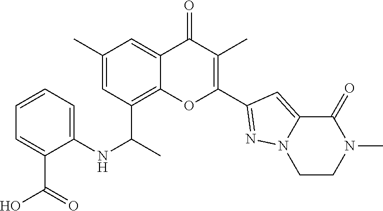 [SMILES: Cc1cc(C(C)Nc2ccccc2C(=O)O)c2oc(-c3cc4n(n3)CCN(C)C4=O)c(C)c(=O)c2c1] | 2-((1-(3,6-dimethyl-2- (5-methyl-4-oxo-4,5,6,7- tetrahydropyrazolo[1,5-a]pyrazin- 2-yl)-4-oxo-4H-chromen-8- yl)ethyl)amino)benzoic acid |
| 74 | 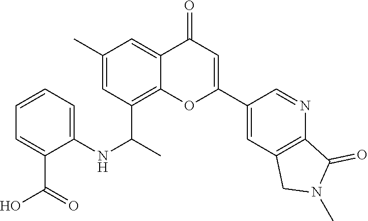 [SMILES: Cc1cc(C(C)Nc2ccccc2C(=O)O)c2oc(-c3cnc4c(c3)CN(C)C4=O)cc(=O)c2c1] | 2-((1-(6-methyl-2-(6-methyl- 7-oxo-6,7-dihydro-5H- pyrrolo[3,4-b]pyridin-3-yl)- 4-oxo-4H-chromen-8- yl)ethyl)amino)benzoic acid |
| 75 | 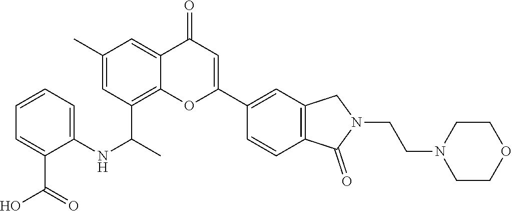 [SMILES: Cc1cc(C(C)Nc2ccccc2C(=O)O)c2oc(-c3ccc4c(c3)CN(CCN3CCOCC3)C4=O)cc(=O)c2c1] | 2-((1-(6-methyl-2-(2-(2- morpholinoethyl)-1-oxoisoindolin-5- yl)-4-oxo-4H-chromen-8- yl)ethyl)amino)benzoic acid |
| 76 | 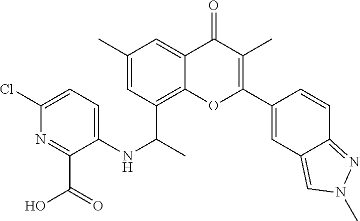 [SMILES: Cc1cc(C(C)Nc2ccc(Cl)nc2C(=O)O)c2oc(-c3ccc4nn(C)cc4c3)c(C)c(=O)c2c1] | 6-chloro-3-((1-(3,6-dimethyl-2- (2-methyl-2H-indazol-5-yl)-4- oxo-4H-chromen-8-yl) ethyl)amino)picolinic acid |
| 77 | 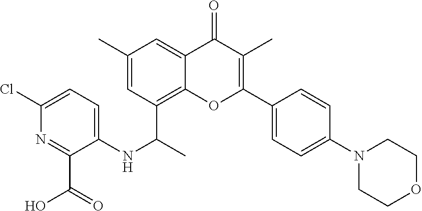 [SMILES: Cc1cc(C(C)Nc2ccc(Cl)nc2C(=O)O)c2oc(-c3ccc(N4CCOCC4)cc3)c(C)c(=O)c2c1] | 6-chloro-3-((1-(3,6-dimethyl-2- (4-morpholinophenyl)-4-oxo- 4H-chromen-8-yl)ethyl) amino)picolinic acid |
| 78 | 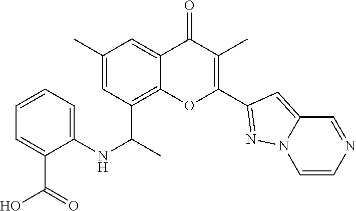 [SMILES: Cc1cc(C(C)Nc2ccccc2C(=O)O)c2oc(-c3cc4cnccn4n3)c(C)c(=O)c2c1] | 2-((1-(3,6-dimethyl-4-oxo-2- (pyrazolo[1,5-a]pyrazin-2-yl)-4H- chromen-8-yl)ethyl) amino)benzoic acid |
| 79 | 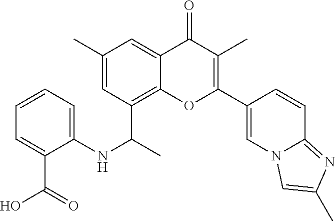 [SMILES: Cc1cc(C(C)Nc2ccccc2C(=O)O)c2oc(-c3ccc4nc(C)cn4c3)c(C)c(=O)c2c1] | 2-((1-(3,6-dimethyl-2-(2- methylimidazo[1,2-a]pyridin-6-yl)-4- oxo-4H-chromen-8-yl) ethyl)amino)benzoic acid |
| 80 | 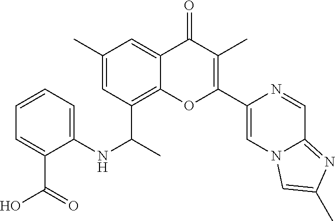 [SMILES: Cc1cc(C(C)Nc2ccccc2C(=O)O)c2oc(-c3cn4cc(C)nc4cn3)c(C)c(=O)c2c1] | 2-((1-(3,6-dimethyl-2-(2- methylimidazo[1,2-a]pyrazin-6-yl)-4- oxo-4H-chromen-8-yl) ethyl)amino)benzoic acid |
| 81 | 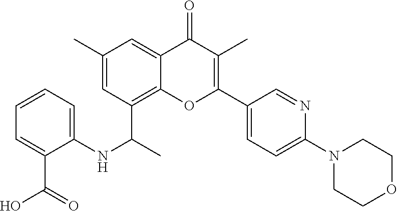 [SMILES: Cc1cc(C(C)Nc2ccccc2C(=O)O)c2oc(-c3ccc(N4CCOCC4)nc3)c(C)c(=O)c2c1] | 2-((1-(3,6-dimethyl-2-(6- morpholinopyridin-3-yl)-4-oxo-4H- chromen-8-yl)ethyl) amino)benzoic acid |
| 82 | 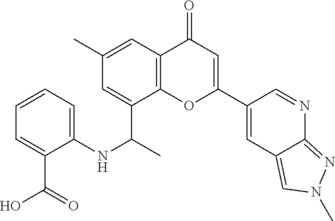 [SMILES: Cc1cc(C(C)Nc2ccccc2C(=O)O)c2oc(-c3cnc4nn(C)cc4c3)cc(=O)c2c1] | 2-((1-(6-methyl-2-(2-methyl- 2H-pyrazolo[3,4-b]pyridin-5-yl)- 4-oxo-4H-chromen-8-yl) ethyl)amino)benzoic acid |
| 83 | 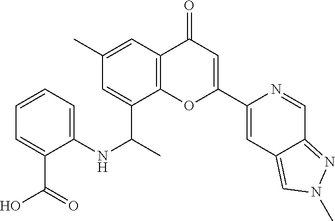 [SMILES: Cc1cc(C(C)Nc2ccccc2C(=O)O)c2oc(-c3cc4cn(C)nc4cn3)cc(=O)c2c1] | 2-((1-(6-methyl-2-(2-methyl- 2H-pyrazolo[3,4-c]pyridin-5-yl)- 4-oxo-4H-chromen-8-yl) ethyl)amino)benzoic acid |
| 84 | 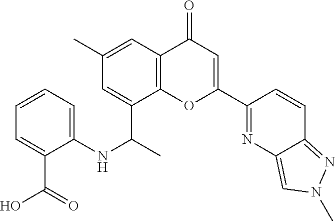 [SMILES: Cc1cc(C(C)Nc2ccccc2C(=O)O)c2oc(-c3ccc4nn(C)cc4n3)cc(=O)c2c1] | 2-((1-(6-methyl-2-(2-methyl-2H- pyrazolo[4,3-b]pyridin-5-yl)- 4-oxo-4H-chromen-8-yl) ethyl)amino)benzoic acid |
| 85 | 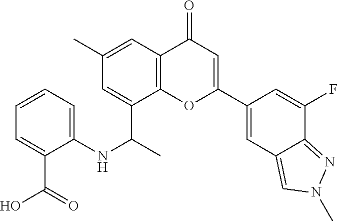 [SMILES: Cc1cc(C(C)Nc2ccccc2C(=O)O)c2oc(-c3cc(F)c4nn(C)cc4c3)cc(=O)c2c1] | 2-((1-(2-(7-fluoro-2-methyl-2H- indazol-5-yl)-6-methyl-4-oxo- 4H-chromen-8-yl)ethyl) amino)benzoic acid |
| 86 | 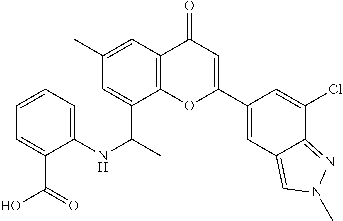 [SMILES: Cc1cc(C(C)Nc2ccccc2C(=O)O)c2oc(-c3cc(Cl)c4nn(C)cc4c3)cc(=O)c2c1] | 2-((1-(2-(7-chloro-2-methyl-2H- indazol-5-yl)-6-methyl-4-oxo- 4H-chromen-8-yl)ethyl) amino)benzoic acid |
| 87 | 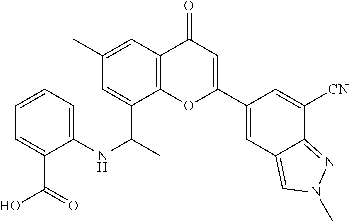 [SMILES: Cc1cc(C(C)Nc2ccccc2C(=O)O)c2oc(-c3cc(C#N)c4nn(C)cc4c3)cc(=O)c2c1] | 2-((1-(2-(7-cyano-2-methyl-2H- indazol-5-yl)-6-methyl-4-oxo- 4H-chromen-8-yl)ethyl) amino)benzoic acid |
| 88 | 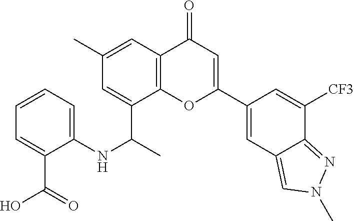 [SMILES: Cc1cc(C(C)Nc2ccccc2C(=O)O)c2oc(-c3cc(C(F)(F)F)c4nn(C)cc4c3)cc(=O)c2c1] | 2-((1-(6-methyl-2-(2-methyl-7- (trifluoromethyl)-2H-indazol-5- yl)-4-oxo-4H-chromen-8-yl) ethyl)amino)benzoic acid |
| 89 | 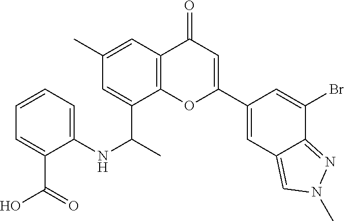 [SMILES: Cc1cc(C(C)Nc2ccccc2C(=O)O)c2oc(-c3cc(Br)c4nn(C)cc4c3)cc(=O)c2c1] | 2-((1-(2-(7-bromo-2-methyl-2H- indazol-5-yl)-6-methyl-4-oxo- 4H-chromen-8-yl)ethyl) amino)benzoic acid |
| 90 | 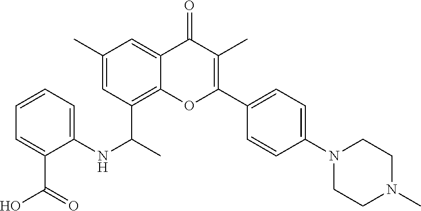 [SMILES: Cc1cc(C(C)Nc2ccccc2C(=O)O)c2oc(-c3ccc(N4CCN(C)CC4)cc3)c(C)c(=O)c2c1] | 2-((1-(3,6-dimethyl-2-(4-(4- methylpiperazin-1-yl)phenyl)-4- oxo-4H-chromen-8-yl) ethyl)amino)benzoic acid |
| 91 | 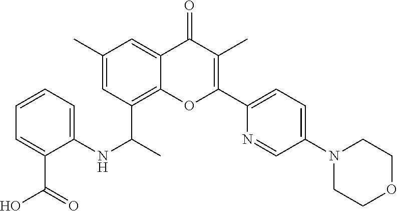 [SMILES: Cc1cc(C(C)Nc2ccccc2C(=O)O)c2oc(-c3ccc(N4CCOCC4)cn3)c(C)c(=O)c2c1] | 2-((1-(3,6-dimethyl-2-(5- morpholinopyridin-2-yl)-4-oxo-4H- chromen-8-yl)ethyl) amino)benzoic acid |
| 92 | 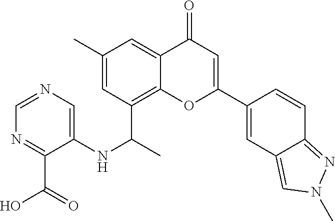 [SMILES: Cc1cc(C(C)Nc2cncnc2C(=O)O)c2oc(-c3ccc4nn(C)cc4c3)cc(=O)c2c1] | 5-((1-(6-methyl-2-(2-methyl- 2H-indazol-5-yl)-4-oxo-4H- chromen-8-yl)ethyl)amino) pyrimidine-4-carboxylic acid |
| 93 | 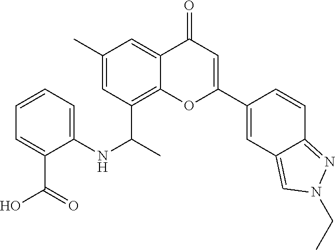 [SMILES: CCn1cc2cc(-c3cc(=O)c4cc(C)cc(C(C)Nc5ccccc5C(=O)O)c4o3)ccc2n1] | 2-((1-(2-(2-ethyl-2H-indazol- 5-yl)-6-methyl-4-oxo-4H- chromen-8-yl)ethyl) amino)benzoic acid |
| 94 | 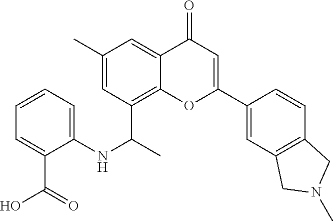 [SMILES: Cc1cc(C(C)Nc2ccccc2C(=O)O)c2oc(-c3ccc4c(c3)CN(C)C4)cc(=O)c2c1] | 2-((1-(6-methyl-2-(2- methylisoindolin-5-yl)-4-oxo-4H- chromen-8-yl)ethyl) amino)benzoic acid |
| 95 | 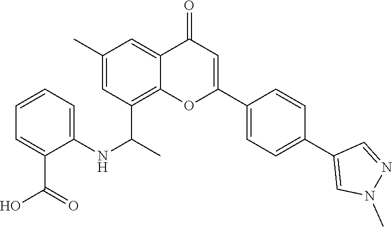 [SMILES: Cc1cc(C(C)Nc2ccccc2C(=O)O)c2oc(-c3ccc(-c4cnn(C)c4)cc3)cc(=O)c2c1] | 2-((1-(6-methyl-2-(4-(1-methyl- 1H-pyrazol-4-yl)phenyl)-4- oxo-4H-chromen-8-yl)ethyl) amino)benzoic acid |
| 96 | 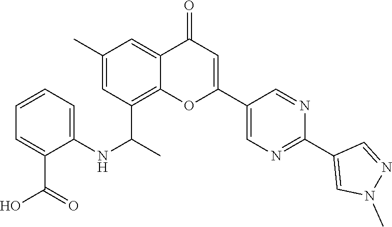 [SMILES: Cc1cc(C(C)Nc2ccccc2C(=O)O)c2oc(-c3cnc(-c4cnn(C)c4)nc3)cc(=O)c2c1] | 2-((1-(6-methyl-2-(2-(1-methyl- 1H-pyrazol-4-yl)pyrimidin-5- yl)-4-oxo-4H-chromen-8-yl) ethyl)amino)benzoic acid |
| 97 | 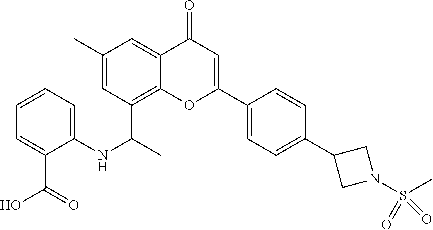 [SMILES: Cc1cc(C(C)Nc2ccccc2C(=O)O)c2oc(-c3ccc(C4CN(S(C)(=O)=O)C4)cc3)cc(=O)c2c1] | 2-((1-(6-methyl-2-(4-(1- (methylsulfonyl) azetidin-3-yl)phenyl)- 4-oxo-4H-chromen-8-yl) ethyl)amino)benzoic acid |
| 98 | 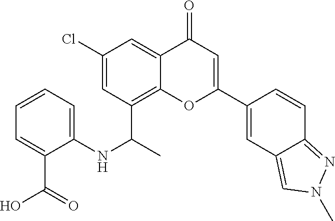 [SMILES: CC(Nc1ccccc1C(=O)O)c1cc(Cl)cc2c(=O)cc(-c3ccc4nn(C)cc4c3)oc12] | 2-((1-(6-chloro-2-(2-methyl- 2H-indazol-5-yl)-4-oxo-4H- chromen-8-yl)ethyl) amino)benzoic acid |
| 99 | 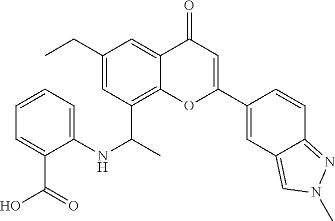 [SMILES: CCc1cc(C(C)Nc2ccccc2C(=O)O)c2oc(-c3ccc4nn(C)cc4c3)cc(=O)c2c1] | 2-((1-(6-ethyl-2-(2-methyl-2H- indazol-5-yl)-4-oxo-4H- chromen-8-yl)ethyl) amino)benzoic acid |
| 100 | 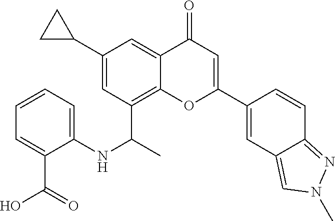 [SMILES: CC(Nc1ccccc1C(=O)O)c1cc(C2CC2)cc2c(=O)cc(-c3ccc4nn(C)cc4c3)oc12] | 2-((1-(6-cyclopropyl-2-(2-methyl- 2H-indazol-5-yl)-4-oxo-4H- chromen-8-yl)ethyl) amino)benzoic acid |
| 101 | 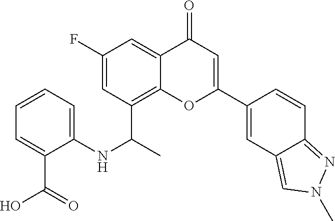 [SMILES: CC(Nc1ccccc1C(=O)O)c1cc(F)cc2c(=O)cc(-c3ccc4nn(C)cc4c3)oc12] | 2-((1-(6-fluoro-2-(2-methyl- 2H-indazol-5-yl)-4-oxo-4H- chromen-8-yl)ethyl) amino)benzoic acid |
| 102 | 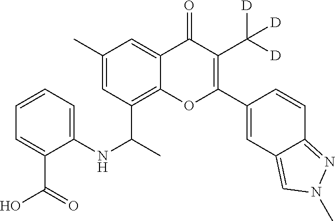 [SMILES: [2H]C([2H])([2H])c1c(-c2ccc3nn(C)cc3c2)oc2c(C(C)Nc3ccccc3C(=O)O)cc(C)cc2c1=O] | 2-((1-(6-methyl-3-(methyl-d3)-2- (2-methyl-2H-indazol-5-yl)-4- oxo-4H-chromen-8-yl) ethyl)amino)benzoic acid |
| 103 | 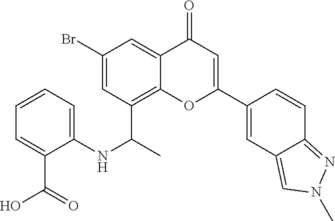 [SMILES: CC(Nc1ccccc1C(=O)O)c1cc(Br)cc2c(=O)cc(-c3ccc4nn(C)cc4c3)oc12] | 2-((1-(6-bromo-2-(2-methyl- 2H-indazol-5-yl)-4-oxo-4H- chromen-8-yl)ethyl) amino)benzoic acid |
| 104 | 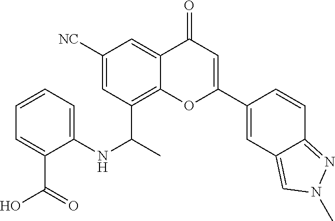 [SMILES: CC(Nc1ccccc1C(=O)O)c1cc(C#N)cc2c(=O)cc(-c3ccc4nn(C)cc4c3)oc12] | 2-((1-(6-cyano-2-(2-methyl- 2H-indazol-5-yl)-4-oxo-4H- chromen-8-yl)ethyl) amino)benzoic acid |
| 105 | 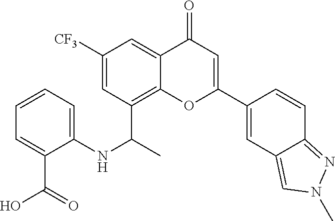 [SMILES: CC(Nc1ccccc1C(=O)O)c1cc(C(F)(F)F)cc2c(=O)cc(-c3ccc4nn(C)cc4c3)oc12] | 2-((1-(2-(2-methyl-2H-indazol-5-yl)-4- oxo-6-(trifluoromethyl)- 4H-chromen-8-yl)ethyl) amino)benzoic acid |
| 106 | 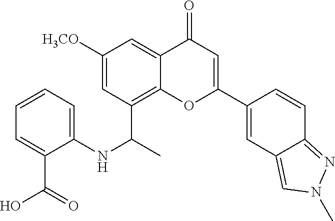 [SMILES: COc1cc(C(C)Nc2ccccc2C(=O)O)c2oc(-c3ccc4nn(C)cc4c3)cc(=O)c2c1] | 2-((1-(6-methoxy-2-(2-methyl- 2H-indazol-5-yl)-4-oxo-4H- chromen-8-yl)ethyl) amino)benzoic acid |
| 107 | 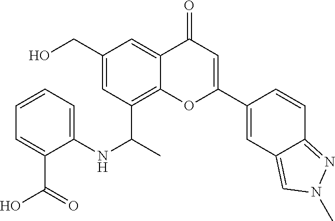 [SMILES: CC(Nc1ccccc1C(=O)O)c1cc(CO)cc2c(=O)cc(-c3ccc4nn(C)cc4c3)oc12] | 2-((1-(6-(hydroxymethyl)-2-(2- methyl-2H-indazol-5-yl)-4-oxo- 4H-chromen-8-yl)ethyl) amino)benzoic acid |
| 108 | 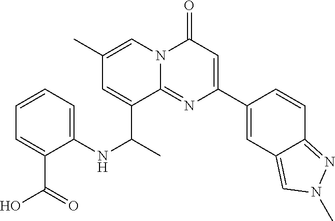 [SMILES: Cc1cc(C(C)Nc2ccccc2C(=O)O)c2nc(-c3ccc4nn(C)cc4c3)cc(=O)n2c1] | 2-((1-(7-methyl-2-(2-methyl- 2H-indazol-5-yl)-4-oxo-4H- pyrido[1,2-a]pyrimidin-9-yl) ethyl)amino)benzoic acid |
| 109 | 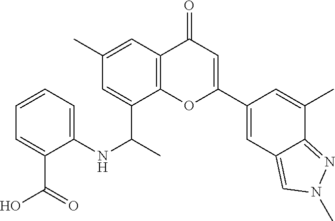 [SMILES: Cc1cc(C(C)Nc2ccccc2C(=O)O)c2oc(-c3cc(C)c4nn(C)cc4c3)cc(=O)c2c1] | 2-((1-(2-(2,7-dimethyl-2H-indazol- 5-yl)-6-methyl-4-oxo-4H- chromen-8-yl)ethyl) amino)benzoic acid |
| 110 | 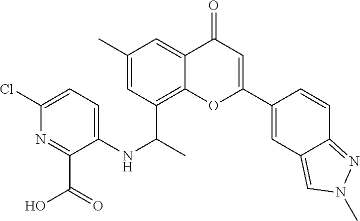 [SMILES: Cc1cc(C(C)Nc2ccc(Cl)nc2C(=O)O)c2oc(-c3ccc4nn(C)cc4c3)cc(=O)c2c1] | 6-chloro-3-((1-(6-methyl-2-(2- methyl-2H-indazol-5-yl)-4-oxo- 4H-chromen-8-yl)ethyl) amino)picolinic acid |
| 111 | 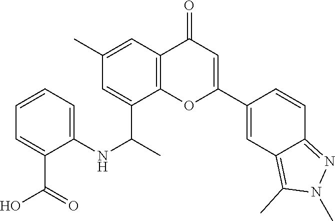 [SMILES: Cc1cc(C(C)Nc2ccccc2C(=O)O)c2oc(-c3ccc4nn(C)c(C)c4c3)cc(=O)c2c1] | 2-((1-(2-(2,3-dimethyl-2H- indazol-5-yl)-6-methyl-4-oxo-4H- chromen-8-yl)ethyl) amino)benzoic acid |
| 112 | 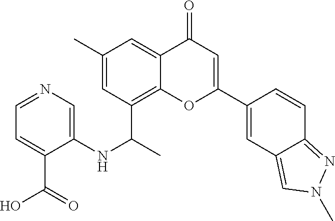 [SMILES: Cc1cc(C(C)Nc2cnccc2C(=O)O)c2oc(-c3ccc4nn(C)cc4c3)cc(=O)c2c1] | 3-((1-(6-methyl-2-(2-methyl- 2H-indazol-5-yl)-4-oxo-4H- chromen-8-yl)ethyl) amino)isonicotinic acid |
| 113 | 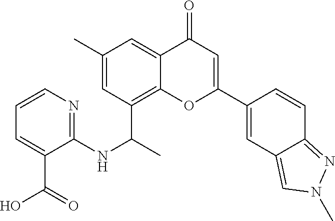 [SMILES: Cc1cc(C(C)Nc2ncccc2C(=O)O)c2oc(-c3ccc4nn(C)cc4c3)cc(=O)c2c1] | 2-((1-(6-methyl-2-(2-methyl- 2H-indazol-5-yl)-4-oxo-4H- chromen-8-yl)ethyl) amino)nicotinic acid |
| 114 | 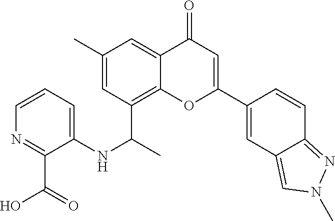 [SMILES: Cc1cc(C(C)Nc2cccnc2C(=O)O)c2oc(-c3ccc4nn(C)cc4c3)cc(=O)c2c1] | 3-((1-(6-methyl-2-(2-methyl- 2H-indazol-5-yl)-4-oxo-4H- chromen-8-yl)ethyl) amino)picolinic acid |
| 115 | 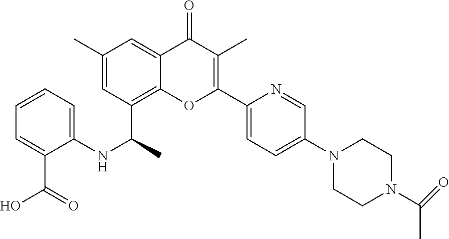 [SMILES: CC(=O)N1CCN(c2ccc(-c3oc4c([C@@H](C)Nc5ccccc5C(=O)O)cc(C)cc4c(=O)c3C)nc2)CC1] | (R)-2-((1-(2-(5-(4-acetylpiperazin- 1-yl)pyridin-2-yl)-3,6- dimethyl-4-oxo-4H-chromen-8- yl)ethyl)amino)benzoic acid |
| 116 | 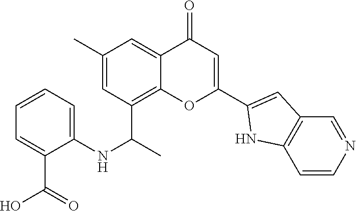 [SMILES: Cc1cc(C(C)Nc2ccccc2C(=O)O)c2oc(-c3cc4cnccc4[nH]3)cc(=O)c2c1] | 2-((1-(6-methyl-4-oxo-2-(1H- pyrrolo[3,2-c]pyridin-2-yl)-4H- chromen-8-yl)ethyl) amino)benzoic acid |
| 117 | 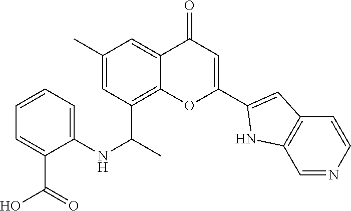 [SMILES: Cc1cc(C(C)Nc2ccccc2C(=O)O)c2oc(-c3cc4ccncc4[nH]3)cc(=O)c2c1] | 2-((1-(6-methyl-4-oxo-2-(1H- pyrrolo[2,3-c]pyridin-2-yl)-4H- chromen-8-yl)ethyl) amino)benzoic acid |
| 118 | 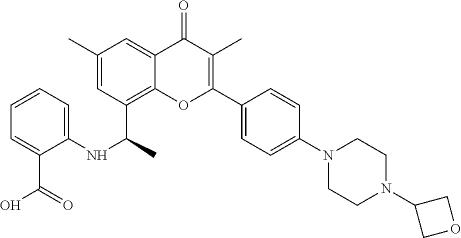 [SMILES: Cc1cc([C@@H](C)Nc2ccccc2C(=O)O)c2oc(-c3ccc(N4CCN(C5COC5)CC4)cc3)c(C)c(=O)c2c1] | (R)-2-((1-(3,6-dimethyl-2-(4- (4-(oxetan-3-yl)piperazin-1- yl)phenyl)-4-oxo-4H-chromen-8- yl)ethyl)amino)benzoic acid |
| 119 | 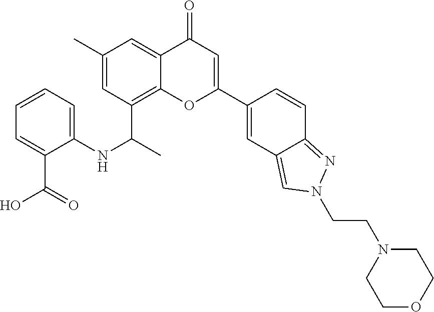 [SMILES: Cc1cc(C(C)Nc2ccccc2C(=O)O)c2oc(-c3ccc4nn(CCN5CCOCC5)cc4c3)cc(=O)c2c1] | 2-((1-(6-methyl-2-(2-(2- morpholinoethyl)-2H-indazol-5-yl)-4- oxo-4H-chromen-8-yl) ethyl)amino)benzoic acid |
| 120 | 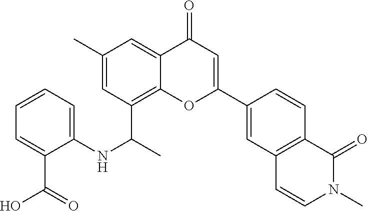 [SMILES: Cc1cc(C(C)Nc2ccccc2C(=O)O)c2oc(-c3ccc4c(=O)n(C)ccc4c3)cc(=O)c2c1] | 2-((1-(6-methyl-2-(2-methyl-1-oxo- 1,2-dihydroisoquinolin-6- yl)-4-oxo-4H-chromen-8-yl) ethyl)amino)benzoic acid |
| 121 | 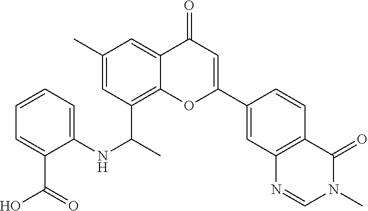 [SMILES: Cc1cc(C(C)Nc2ccccc2C(=O)O)c2oc(-c3ccc4c(=O)n(C)cnc4c3)cc(=O)c2c1] | 2-((1-(6-methyl-2-(3-methyl-4- oxo-3,4-dihydroquinazolin-7- yl)-4-oxo-4H-chromen-8-yl) ethyl)amino)benzoic acid |
| 122 | 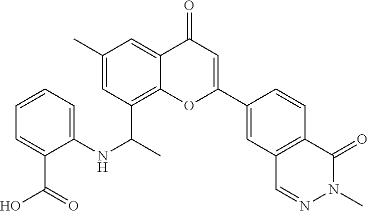 [SMILES: Cc1cc(C(C)Nc2ccccc2C(=O)O)c2oc(-c3ccc4c(=O)n(C)ncc4c3)cc(=O)c2c1] | 2-((1-(6-methyl-2-(2-methyl-1- oxo-1,2-dihydrophthalazin-6- yl)-4-oxo-4H-chromen-8-yl) ethyl)amino)benzoic acid |
| 123 | 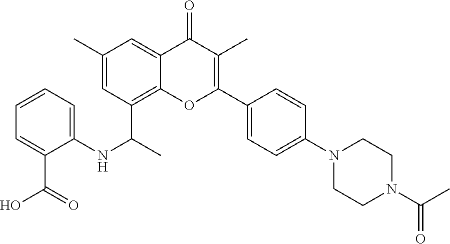 [SMILES: CC(=O)N1CCN(c2ccc(-c3oc4c(C(C)Nc5ccccc5C(=O)O)cc(C)cc4c(=O)c3C)cc2)CC1] | 2-((1-(2-(4-(4-acetylpiperazin-1- yl)phenyl)-3,6-dimethyl-4- oxo-4H-chromen-8-yl)ethyl) amino)benzoic acid |
| 124 | 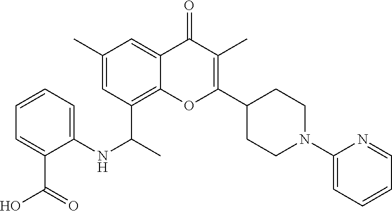 [SMILES: Cc1cc(C(C)Nc2ccccc2C(=O)O)c2oc(C3CCN(c4ccccn4)CC3)c(C)c(=O)c2c1] | 2-((1-(3,6-dimethyl-4-oxo-2-(1- (pyridin-2-yl)piperidin-4-yl)- 4H-chromen-8-yl)ethyl) amino)benzoic acid |
| 125 | 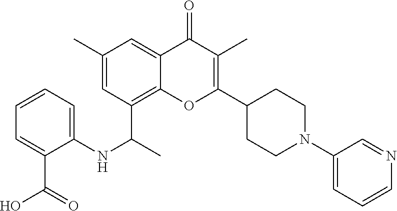 [SMILES: Cc1cc(C(C)Nc2ccccc2C(=O)O)c2oc(C3CCN(c4cccnc4)CC3)c(C)c(=O)c2c1] | 2-((1-(3,6-dimethyl-4-oxo-2-(1- (pyridin-3-yl)piperidin-4-yl)- 4H-chromen-8-yl)ethyl) amino)benzoic acid |
| 126 | 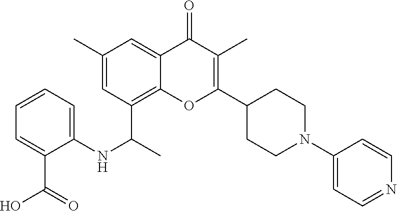 [SMILES: Cc1cc(C(C)Nc2ccccc2C(=O)O)c2oc(C3CCN(c4ccncc4)CC3)c(C)c(=O)c2c1] | 2-((1-(3,6-dimethyl-4-oxo-2-(1- (pyridin-4-yl)piperidin-4-yl)- 4H-chromen-8-yl)ethyl) amino)benzoic acid |
| 127 | 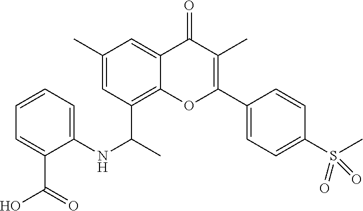 [SMILES: Cc1cc(C(C)Nc2ccccc2C(=O)O)c2oc(-c3ccc(S(C)(=O)=O)cc3)c(C)c(=O)c2c1] | 2-((1-(3,6-dimethyl-2-(4- (methylsulfonyl)phenyl)-4-oxo-4H- chromen-8-yl)ethyl) amino)benzoic acid |
| 128 | 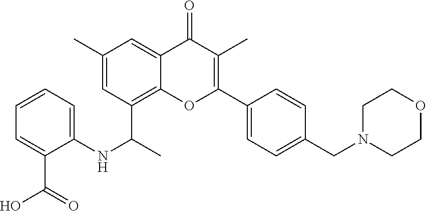 [SMILES: Cc1cc(C(C)Nc2ccccc2C(=O)O)c2oc(-c3ccc(CN4CCOCC4)cc3)c(C)c(=O)c2c1] | 2-((1-(3,6-dimethyl-2-(4- (morpholinomethyl)phenyl)-4-oxo- 4H-chromen-8-yl)ethyl) amino)benzoic acid |
| 129 | 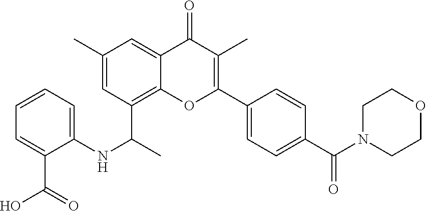 [SMILES: Cc1cc(C(C)Nc2ccccc2C(=O)O)c2oc(-c3ccc(C(=O)N4CCOCC4)cc3)c(C)c(=O)c2c1] | 2-((1-(3,6-dimethyl-2-(4- (morpholine-4-carbonyl)phenyl)-4- oxo-4H-chromen-8-yl) ethyl)amino)benzoic acid |
| 130 | 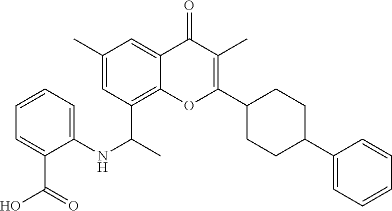 [SMILES: Cc1cc(C(C)Nc2ccccc2C(=O)O)c2oc(C3CCC(c4ccccc4)CC3)c(C)c(=O)c2c1] | 2-((1-(3,6-dimethyl-4-oxo- 2-(4-phenylcyclohexyl)-4H- chromen-8-yl)ethyl) amino)benzoic acid |
| 131 | 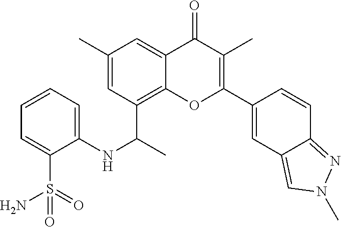 [SMILES: Cc1cc(C(C)Nc2ccccc2S(N)(=O)=O)c2oc(-c3ccc4nn(C)cc4c3)c(C)c(=O)c2c1] | 2-((1-(3,6-dimethyl-2-(2-methyl- 2H-indazol-5-yl)-4-oxo-4H- chromen-8-yl)ethyl)amino) benzenesulfonamide |
| 132 | 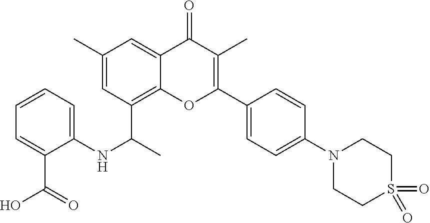 [SMILES: Cc1cc(C(C)Nc2ccccc2C(=O)O)c2oc(-c3ccc(N4CCS(=O)(=O)CC4)cc3)c(C)c(=O)c2c1] | 2-((1-(2-(4-(1,1-dioxidothiomorpholino) phenyl)-3,6-dimethyl- 4-oxo-4H-chromen-8-yl) ethyl)amino)benzoic acid |
| 133 | 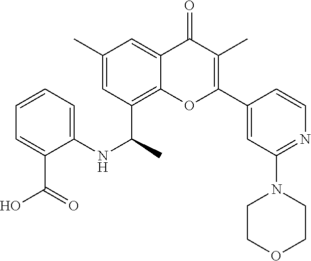 [SMILES: Cc1cc([C@@H](C)Nc2ccccc2C(=O)O)c2oc(-c3ccnc(N4CCOCC4)c3)c(C)c(=O)c2c1] | (R)-2-((1-(3,6-dimethyl-2-(2- morpholinopyridin-4-yl)-4-oxo- 4H-chromen-8-yl)ethyl) amino)benzoic acid |
| 134 | 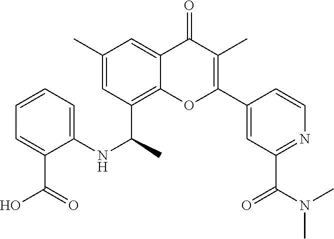 [SMILES: Cc1cc([C@@H](C)Nc2ccccc2C(=O)O)c2oc(-c3ccnc(C(=O)N(C)C)c3)c(C)c(=O)c2c1] | (R)-2-((1-(2-(2-(dimethylcarbamoyl) pyridin-4-yl)-3,6- dimethyl-4-oxo-4H-chromen-8-yl) ethyl)amino)benzoic acid |
| 135 | 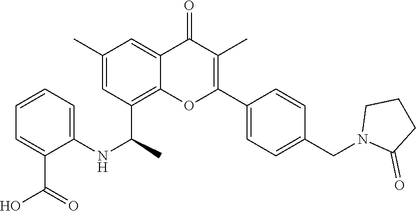 [SMILES: Cc1cc([C@@H](C)Nc2ccccc2C(=O)O)c2oc(-c3ccc(CN4CCCC4=O)cc3)c(C)c(=O)c2c1] | (R)-2-((1-(3,6-dimethyl-4-oxo- 2-(4-((2-oxopyrrolidin-1- yl)methyl)phenyl)-4H-chromen-8- yl)ethyl)amino)benzoic acid |
| 136 | 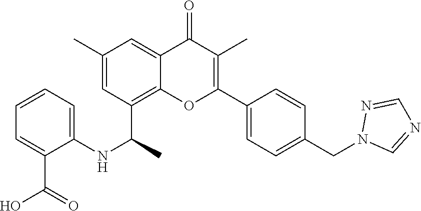 [SMILES: Cc1cc([C@@H](C)Nc2ccccc2C(=O)O)c2oc(-c3ccc(Cn4cncn4)cc3)c(C)c(=O)c2c1] | (R)-2-((1-(2-(4-((1H-1,2,4-triazol- 1-yl)methyl)phenyl)-3,6- dimethyl-4-oxo-4H-chromen-8- yl)ethyl)amino)benzoic acid |
| 137 | 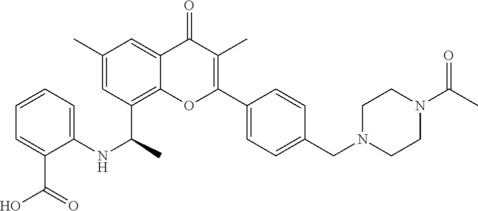 [SMILES: CC(=O)N1CCN(Cc2ccc(-c3oc4c([C@@H](C)Nc5ccccc5C(=O)O)cc(C)cc4c(=O)c3C)cc2)CC1] | (R)-2-((1-(2-(4-((4-acetylpiperazin- 1-yl)methyl)phenyl)-3,6- dimethyl-4-oxo-4H-chromen-8- yl)ethyl)amino)benzoic acid |
| 138 | 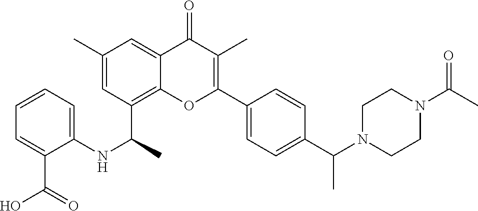 [SMILES: CC(=O)N1CCN(C(C)c2ccc(-c3oc4c([C@@H](C)Nc5ccccc5C(=O)O)cc(C)cc4c(=O)c3C)cc2)CC1] | 2-(((1R)-1-(2-(4-(1-(4-acetylpiperazin- 1-yl)ethyl)phenyl)-3,6- dimethyl-4-oxo-4H-chromen-8- yl)ethyl)amino)benzoic acid |
| 139 | 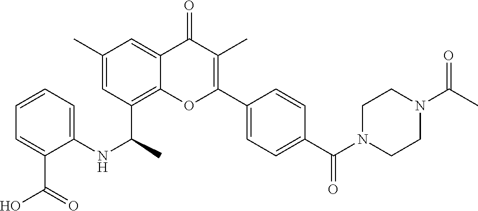 [SMILES: CC(=O)N1CCN(C(=O)c2ccc(-c3oc4c([C@@H](C)Nc5ccccc5C(=O)O)cc(C)cc4c(=O)c3C)cc2)CC1] | (R)-2-((1-(2-(4-(4-acetylpiperazine- 1-carbonyl)phenyl)-3,6- dimethyl-4-oxo-4H-chromen-8- yl)ethyl)amino)benzoic acid |
| 140 | 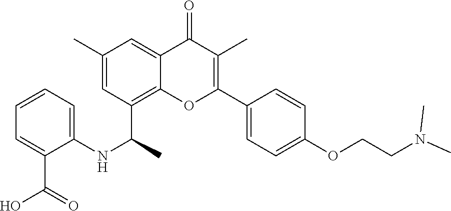 [SMILES: Cc1cc([C@@H](C)Nc2ccccc2C(=O)O)c2oc(-c3ccc(OCCN(C)C)cc3)c(C)c(=O)c2c1] | (R)-2-((1-(2-(4-(2-(dimethylamino) ethoxy)phenyl)-3,6- dimethyl-4-oxo-4H-chromen-8-yl) ethyl)amino)benzoic acid |
| 141 | 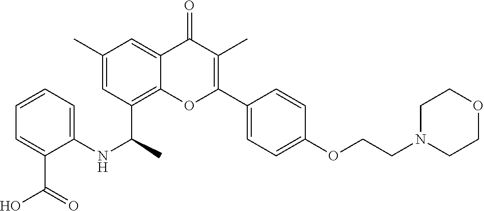 [SMILES: Cc1cc([C@@H](C)Nc2ccccc2C(=O)O)c2oc(-c3ccc(OCCN4CCOCC4)cc3)c(C)c(=O)c2c1] | (R)-2-((1-(3,6-dimethyl-2-(4-(2- morpholinoethoxy)phenyl)-4- oxo-4H-chromen-8-yl)ethyl) amino)benzoic acid |
| 142 | 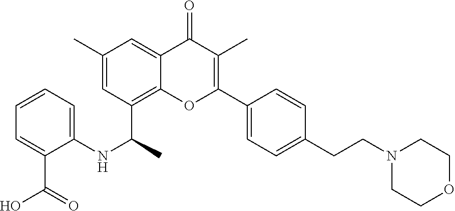 [SMILES: Cc1cc([C@@H](C)Nc2ccccc2C(=O)O)c2oc(-c3ccc(CCN4CCOCC4)cc3)c(C)c(=O)c2c1] | (R)-2-((1-(3,6-dimethyl-2-(4- (2-morpholinoethyl)phenyl)-4- oxo-4H-chromen-8-yl) ethyl)amino)benzoic acid |
| 143 | 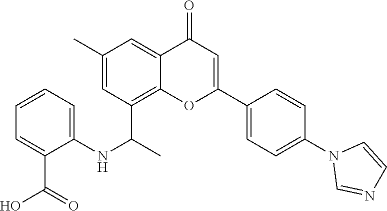 [SMILES: Cc1cc(C(C)Nc2ccccc2C(=O)O)c2oc(-c3ccc(-n4ccnc4)cc3)cc(=O)c2c1] | 2-((1-(2-(4-(1H-imidazol-1-yl) phenyl)-6-methyl-4-oxo-4H- chromen-8-yl)ethyl) amino)benzoic acid |
| 144 | 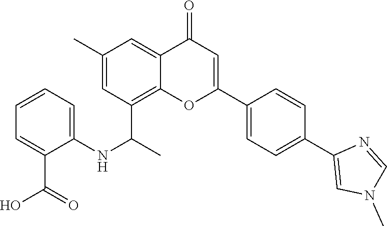 [SMILES: Cc1cc(C(C)Nc2ccccc2C(=O)O)c2oc(-c3ccc(-c4cn(C)cn4)cc3)cc(=O)c2c1] | 2-((1-(6-methyl-2-(4-(1-methyl- 1H-imidazol-4-yl)phenyl)-4- oxo-4H-chromen-8-yl) ethyl)amino)benzoic acid |
| 145 | 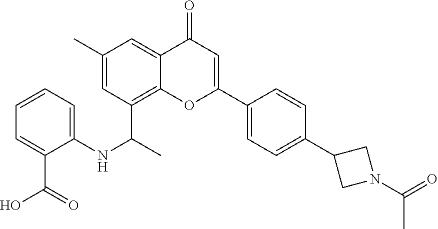 [SMILES: CC(=O)N1CC(c2ccc(-c3cc(=O)c4cc(C)cc(C(C)Nc5ccccc5C(=O)O)c4o3)cc2)C1] | 2-((1-(2-(4-(1-acetylazetidin-3-yl) phenyl)-6-methyl-4-oxo-4H- chromen-8-yl)ethyl) amino)benzoic acid |
| 146 | 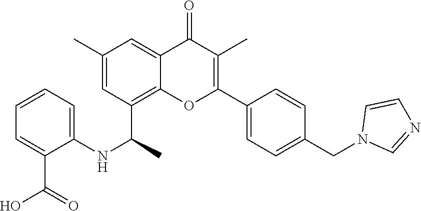 [SMILES: Cc1cc([C@@H](C)Nc2ccccc2C(=O)O)c2oc(-c3ccc(Cn4ccnc4)cc3)c(C)c(=O)c2c1] | (R)-2-((1-(2-(4-((1H-imidazol- 1-yl)methyl)phenyl)-3,6- dimethyl-4-oxo-4H-chromen-8- yl)ethyl)amino)benzoic acid |
| 147 | 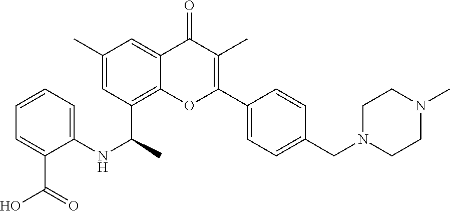 [SMILES: Cc1cc([C@@H](C)Nc2ccccc2C(=O)O)c2oc(-c3ccc(CN4CCN(C)CC4)cc3)c(C)c(=O)c2c1] | (R)-2-((1-(3,6-dimethyl-2- (4-((4-methylpiperazin-1- yl)methyl)phenyl)-4- oxo-4H-chromen-8- yl)ethyl)amino)benzoic acid |
| 148 | 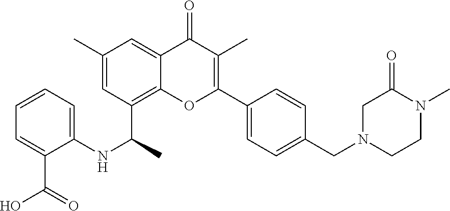 [SMILES: Cc1cc([C@@H](C)Nc2ccccc2C(=O)O)c2oc(-c3ccc(CN4CCN(C)C(=O)C4)cc3)c(C)c(=O)c2c1] | (R)-2-((1-(3,6-dimethyl-2-(4- ((4-methyl-3-oxopiperazin-1- yl)methyl)phenyl)-4- oxo-4H-chromen-8- yl)ethyl)amino)benzoic acid |
| 149 | 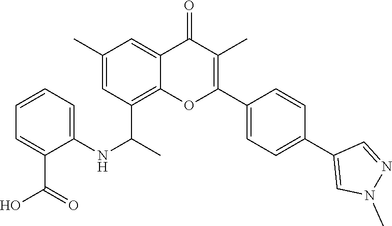 [SMILES: Cc1cc(C(C)Nc2ccccc2C(=O)O)c2oc(-c3ccc(-c4cnn(C)c4)cc3)c(C)c(=O)c2c1] | 2-((1-(3,6-dimethyl-2-(4-(1- methyl-1H-pyrazol-4-yl)phenyl)-4- oxo-4H-chromen-8-yl) ethyl)amino)benzoic acid |
| 150 | 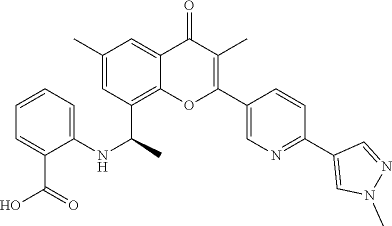 [SMILES: Cc1cc([C@@H](C)Nc2ccccc2C(=O)O)c2oc(-c3ccc(-c4cnn(C)c4)nc3)c(C)c(=O)c2c1] | (R)-2-((1-(3,6-dimethyl-2- (6-(1-methyl-1H-pyrazol-4- yl)pyridin-3-yl)-4-oxo-4H- chromen-8-yl)ethyl)amino)benzoic acid |
| 151 | 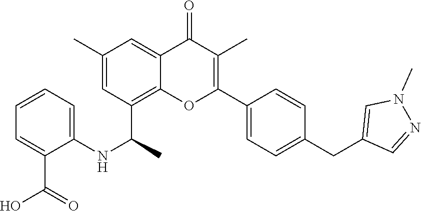 [SMILES: Cc1cc([C@@H](C)Nc2ccccc2C(=O)O)c2oc(-c3ccc(Cc4cnn(C)c4)cc3)c(C)c(=O)c2c1] | (R)-2-((1-(3,6-dimethyl-2- (4-((1-methyl-1H-pyrazol-4- yl)methyl)phenyl)-4- oxo-4H-chromen-8- yl)ethyl)amino)benzoic acid |
| 152 | 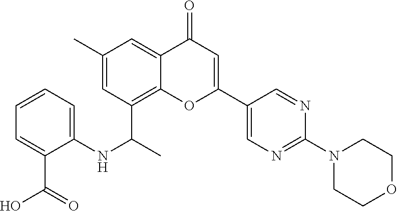 [SMILES: Cc1cc(C(C)Nc2ccccc2C(=O)O)c2oc(-c3cnc(N4CCOCC4)nc3)cc(=O)c2c1] | 2-((1-(6-methyl-2-(2- morpholinopyrimidin-5-yl)-4-oxo-4H- chromen-8-yl)ethyl) amino)benzoic acid |
| 153 | 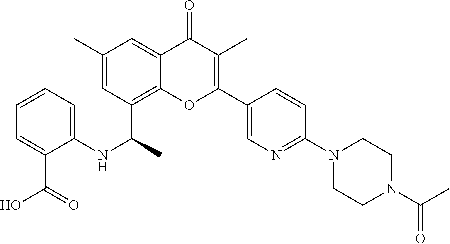 [SMILES: CC(=O)N1CCN(c2ccc(-c3oc4c([C@@H](C)Nc5ccccc5C(=O)O)cc(C)cc4c(=O)c3C)cn2)CC1] | (R)-2-((1-(2-(6-(4-acetylpiperazin- 1-yl)pyridin-3-yl)-3,6- dimethyl-4-oxo-4H-chromen- 8-yl)ethyl)amino)benzoic acid |
| 154 | 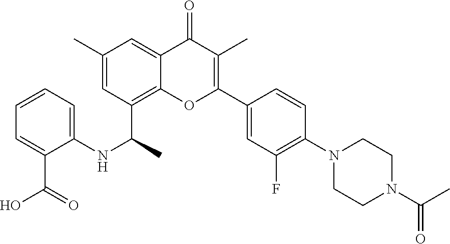 [SMILES: CC(=O)N1CCN(c2ccc(-c3oc4c([C@@H](C)Nc5ccccc5C(=O)O)cc(C)cc4c(=O)c3C)cc2F)CC1] | (R)-2-((1-(2-(4-(4-acetylpiperazin- 1-yl)-3-fluorophenyl)-3,6- dimethyl-4-oxo-4H-chromen- 8-yl)ethyl)amino)benzoic acid |
| 155 | 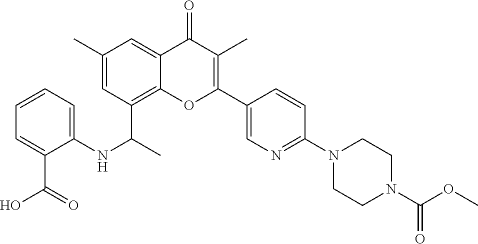 [SMILES: COC(=O)N1CCN(c2ccc(-c3oc4c(C(C)Nc5ccccc5C(=O)O)cc(C)cc4c(=O)c3C)cn2)CC1] | 2-((1-(2-(6-(4-(methoxycarbonyl) piperazin-1-yl)pyridin-3-yl)- 3,6-dimethyl-4-oxo-4H-chromen- 8-yl)ethyl)amino)benzoic acid |
| 156 | 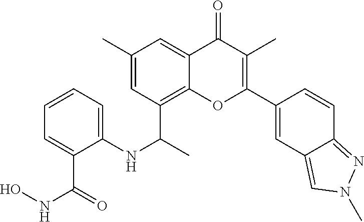 [SMILES: Cc1cc(C(C)Nc2ccccc2C(=O)NO)c2oc(-c3ccc4nn(C)cc4c3)c(C)c(=O)c2c1] | 2-((1-(3,6-dimethyl-2-(2-methyl- 2H-indazol-5-yl)-4-oxo-4H- chromen-8-yl)ethyl)amino)- N-hydroxybenzamide |
| 157 | 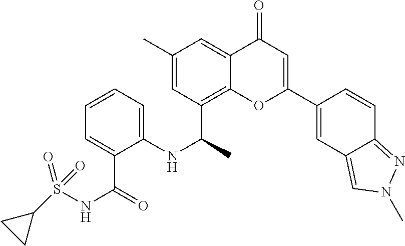 [SMILES: Cc1cc([C@@H](C)Nc2ccccc2C(=O)NS(=O)(=O)C2CC2)c2oc(-c3ccc4nn(C)cc4c3)cc(=O)c2c1] | (R)-N-(cyclopropylsulfonyl)-2- ((1-(6-methyl-2-(2-methyl-2H- indazol-5-yl)-4-oxo-4H-chromen- 8-yl)ethyl)amino)benzamide |
| 158 | 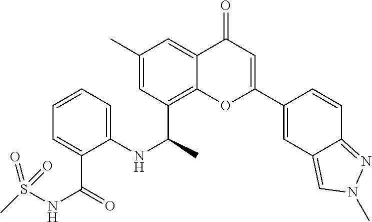 | (R)-2-((1-(6-methyl-2-(2-methyl- 2H-indazol-5-yl)-4-oxo-4H- chromen-8-yl)ethyl)amino)-N- (methylsulfonyl)benzamide |
| 159 | 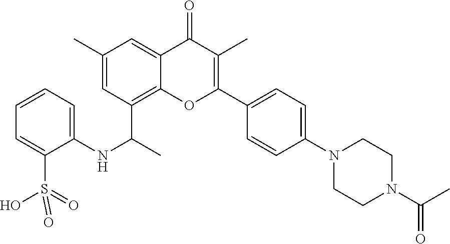 [SMILES: CC(=O)N1CCN(c2ccc(-c3oc4c(C(C)Nc5ccccc5S(=O)(=O)O)cc(C)cc4c(=O)c3C)cc2)CC1] | 2-((1-(2-(4-(4-acetylpiperazin- 1-yl)phenyl)-3,6-dimethyl-4- oxo-4H-chromen-8-yl)ethyl) amino)benzenesulfonamide |
| 160 | 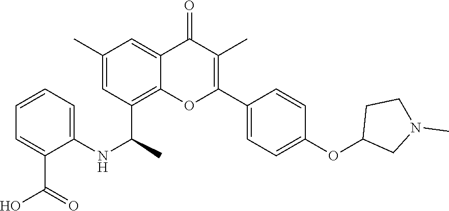 [SMILES: Cc1cc([C@@H](C)Nc2ccccc2C(=O)O)c2oc(-c3ccc(OC4CCN(C)C4)cc3)c(C)c(=O)c2c1] | 2-(((1R)-1-(3,6-dimethyl-2-(4- ((1-methylpyrrolidin-3- yl)oxy)phenyl)-4-oxo-4H-chromen- 8-yl)ethyl)amino)benzoic acid |
| 161 | 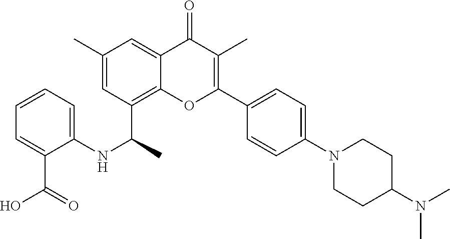 [SMILES: Cc1cc([C@@H](C)Nc2ccccc2C(=O)O)c2oc(-c3ccc(N4CCC(N(C)C)CC4)cc3)c(C)c(=O)c2c1] | (R)-2-((1-(2-(4-(4-(dimethylamino) piperidin-1-yl)phenyl)-3,6- dimethyl-4-oxo-4H-chromen- 8-yl)ethyl)amino)benzoic acid |
| 162 | 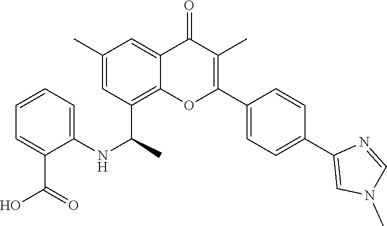 [SMILES: Cc1cc([C@@H](C)Nc2ccccc2C(=O)O)c2oc(-c3ccc(-c4cn(C)cn4)cc3)c(C)c(=O)c2c1] | (R)-2-((1-(3,6-dimethyl-2-(4- (1-methyl-1H-imidazol-4- yl)phenyl)-4-oxo-4H-chromen- 8-yl)ethyl)amino)benzoic acid |
| 163 | 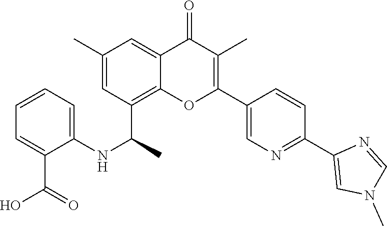 [SMILES: Cc1cc([C@@H](C)Nc2ccccc2C(=O)O)c2oc(-c3ccc(-c4cn(C)cn4)nc3)c(C)c(=O)c2c1] | (R)-2-((1-(3,6-dimethyl-2-(6- (1-methyl-1H-imidazol-4- yl)pyridin-3-yl)-4-oxo-4H-chromen- 8-yl)ethyl)amino)benzoic acid |
| 164 | 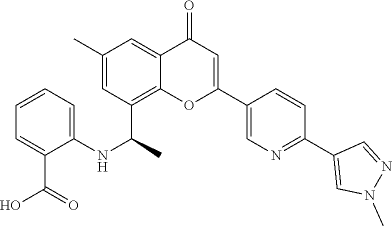 [SMILES: Cc1cc([C@@H](C)Nc2ccccc2C(=O)O)c2oc(-c3ccc(-c4cnn(C)c4)nc3)cc(=O)c2c1] | (R)-2-((1-(6-methyl-2-(6-(1- methyl-1H-pyrazol-4-yl)pyridin-3- yl)-4-oxo-4H-chromen-8-yl) ethyl)amino)benzoic acid |
| 165 | 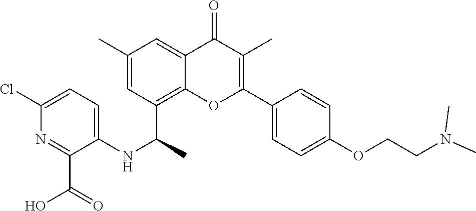 [SMILES: Cc1cc([C@@H](C)Nc2ccc(Cl)nc2C(=O)O)c2oc(-c3ccc(OCCN(C)C)cc3)c(C)c(=O)c2c1] | (R)-6-chloro-3-((1-(2-(4-(2- (dimethylamino)ethoxy)phenyl)- 3,6-dimethyl-4-oxo-4H-chromen- 8-yl)ethyl)amino)picolinic acid |
| 166 | 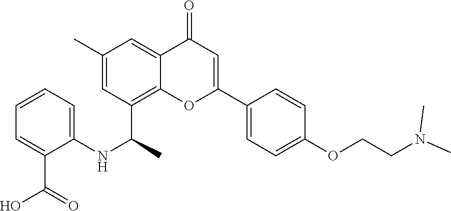 [SMILES: Cc1cc([C@@H](C)Nc2ccccc2C(=O)O)c2oc(-c3ccc(OCCN(C)C)cc3)cc(=O)c2c1] | (R)-2-((1-(2-(4-(2-(dimethylamino) ethoxy)phenyl)-6-methyl-4- oxo-4H-chromen-8-yl) ethyl)amino)benzoic acid |
| 167 | 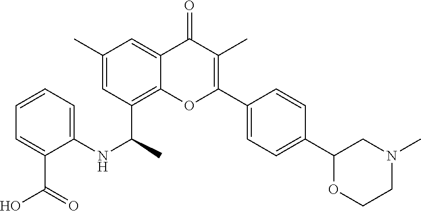 [SMILES: Cc1cc([C@@H](C)Nc2ccccc2C(=O)O)c2oc(-c3ccc(C4CN(C)CCO4)cc3)c(C)c(=O)c2c1] | 2-(((1R)-1-(3,6-dimethyl-2- (4-(4-methylmorpholin-2- yl)phenyl)-4-oxo-4H-chromen- 8-yl)ethyl)amino)benzoic acid |
| 168 | 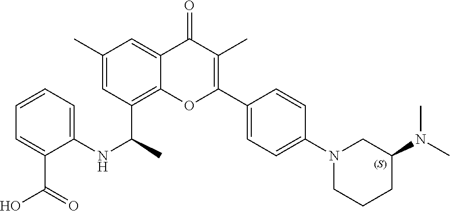 | 2-(((R)-1-(2-(4-((S)-3- (dimethylamino)piperidin-1-yl)phenyl)- 3,6-dimethyl-4-oxo-4H-chromen- 8-yl)ethyl)amino)benzoic acid |
| 169 | 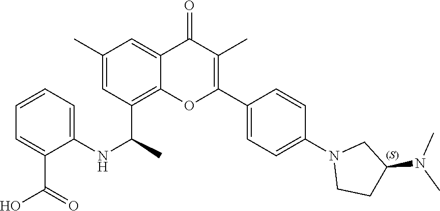 | 2-(((R)-1-(2-(4-((S)-3-(dimethylamino) pyrrolidin-1-yl)phenyl)- 3,6-dimethyl-4-oxo-4H-chromen- 8-yl)ethyl)amino)benzoic acid |
| 170 | 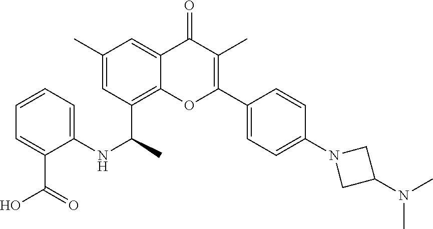 [SMILES: Cc1cc([C@@H](C)Nc2ccccc2C(=O)O)c2oc(-c3ccc(N4CC(N(C)C)C4)cc3)c(C)c(=O)c2c1] | (R)-2-((1-(2-(4-(3-(dimethylamino) azetidin-1-yl)phenyl)-3,6- dimethyl-4-oxo-4H-chromen-8-yl) ethyl)amino)benzoic acid |
| 171 | 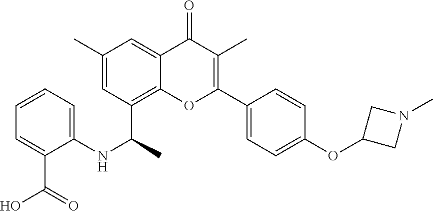 [SMILES: Cc1cc([C@@H](C)Nc2ccccc2C(=O)O)c2oc(-c3ccc(OC4CN(C)C4)cc3)c(C)c(=O)c2c1] | (R)-2-((1-(3,6-dimethyl-2- (4-((1-methylazetidin-3- yl)oxy)phenyl)-4-oxo-4H-chromen- 8-yl)ethyl)amino)benzoic acid |
| 172 | 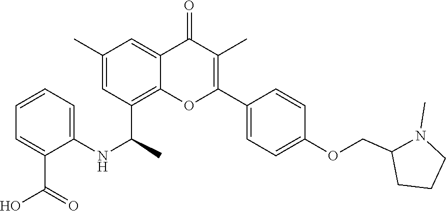 [SMILES: Cc1cc([C@@H](C)Nc2ccccc2C(=O)O)c2oc(-c3ccc(OCC4CCCN4C)cc3)c(C)c(=O)c2c1] | 2-(((1R)-1-(3,6-dimethyl-2- (4-((1-methylpyrrolidin-2- yl)methoxy)phenyl)- 4-oxo-4H-chromen-8- yl)ethyl)amino)benzoic acid |
| 173 | 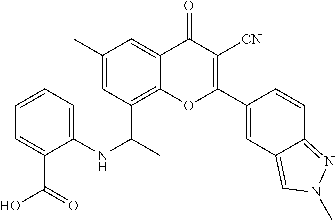 [SMILES: Cc1cc(C(C)Nc2ccccc2C(=O)O)c2oc(-c3ccc4nn(C)cc4c3)c(C#N)c(=O)c2c1] | 2-((1-(3-cyano-6-methyl-2-(2- methyl-2H-indazol-5-yl)-4-oxo- 4H-chromen-8-yl)ethyl) amino)benzoic acid |
| 174 | 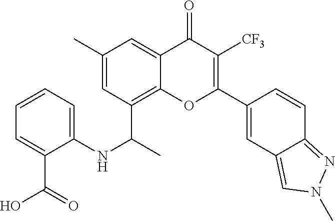 [SMILES: Cc1cc(C(C)Nc2ccccc2C(=O)O)c2oc(-c3ccc4nn(C)cc4c3)c(C(F)(F)F)c(=O)c2c1] | 2-((1-(6-methyl-2-(2-methyl- 2H-indazol-5-yl)-4-oxo-3- (trifluoromethyl)-4H-chromen-8-yl) ethyl)amino)benzoic acid |
| 175 | 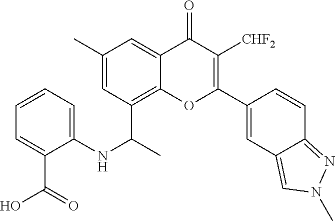 [SMILES: Cc1cc(C(C)Nc2ccccc2C(=O)O)c2oc(-c3ccc4nn(C)cc4c3)c(C(F)F)c(=O)c2c1] | 2-((1-(3-(difluoromethyl)-6- methyl-2-(2-methyl-2H-indazol-5- yl)-4-oxo-4H-chromen-8-yl) ethyl)amino)benzoic acid |
| 176 | 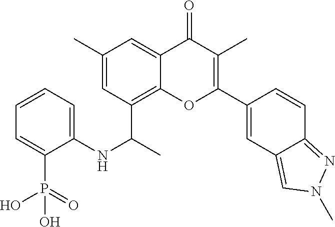 [SMILES: Cc1cc(C(C)Nc2ccccc2P(=O)(O)O)c2oc(-c3ccc4nn(C)cc4c3)c(C)c(=O)c2c1] | (2-((1-(3,6-dimethyl-2-(2-methyl- 2H-indazol-5-yl)-4-oxo-4H- chromen-8-yl)ethyl)amino) phenyl)phosphonic acid |
| 177 | 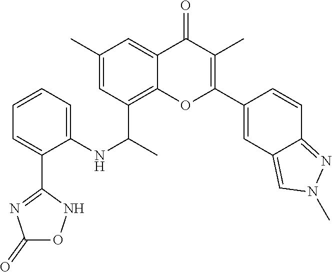 [SMILES: Cc1cc(C(C)Nc2ccccc2-c2nc(=O)o[nH]2)c2oc(-c3ccc4nn(C)cc4c3)c(C)c(=O)c2c1] | 3-(2-((1-(3,6-dimethyl-2-(2- methyl-2H-indazol-5-yl)-4-oxo- 4H-chromen-8-yl)ethyl)amino) phenyl)-1,2,4-oxadiazol-5(2H)- one |
| 178 | 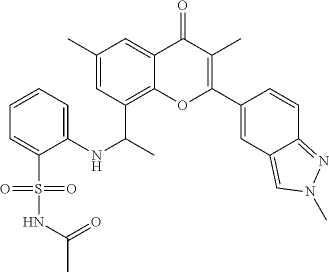 [SMILES: CC(=O)NS(=O)(=O)c1ccccc1NC(C)c1cc(C)cc2c(=O)c(C)c(-c3ccc4nn(C)cc4c3)oc12] | N-((2-((1-(3,6-dimethyl-2-(2- methyl-2H-indazol-5-yl)-4-oxo- 4H-chromen-8-yl)ethyl)amino) phenyl)sulfonyl)acetamide |
| 179 | 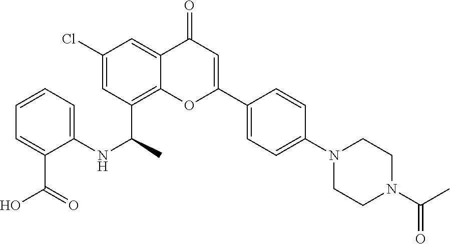 [SMILES: CC(=O)N1CCN(c2ccc(-c3cc(=O)c4cc(Cl)cc([C@@H](C)Nc5ccccc5C(=O)O)c4o3)cc2)CC1] | (R)-2-((1-(2-(4-(4-acetylpiperazin- 1-yl)phenyl)-6-chloro-4-oxo- 4H-chromen-8-yl)ethyl) amino)benzoic acid |
| 180 | 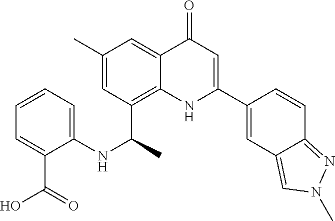 [SMILES: Cc1cc([C@@H](C)Nc2ccccc2C(=O)O)c2[nH]c(-c3ccc4nn(C)cc4c3)cc(=O)c2c1] | (R)-2-((1-(6-methyl-2-(2-methyl- 2H-indazol-5-yl)-4-oxo-1,4- dihydroquinolin-8-yl) ethyl)amino)benzoic acid |
| 181 | 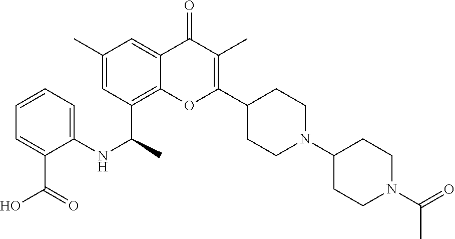 [SMILES: CC(=O)N1CCC(N2CCC(c3oc4c([C@@H](C)Nc5ccccc5C(=O)O)cc(C)cc4c(=O)c3C)CC2)CC1] | (R)-2-((1-(2-(1'-acetyl-[1,4'- bipiperidin]-4-yl)-3,6-dimethyl-4- oxo-4H-chromen-8-yl) ethyl)amino)benzoic acid |
| 182 | 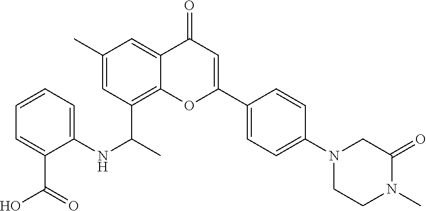 [SMILES: Cc1cc(C(C)Nc2ccccc2C(=O)O)c2oc(-c3ccc(N4CCN(C)C(=O)C4)cc3)cc(=O)c2c1] | 2-((1-(6-methyl-2-(4-(4-methyl-3- oxopiperazin-1-yl)phenyl)-4- oxo-4H-chromen-8-yl) ethyl)amino)benzoic acid |
| 183 | 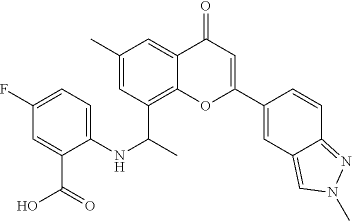 [SMILES: Cc1cc(C(C)Nc2ccc(F)cc2C(=O)O)c2oc(-c3ccc4nn(C)cc4c3)cc(=O)c2c1] | 5-fluoro-2-((1-(6-methyl-2-(2- methyl-2H-indazol-5-yl)-4-oxo- 4H-chromen-8-yl) ethyl)amino)benzoic acid |
| 184 | 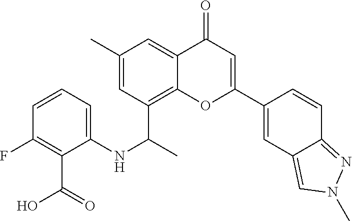 [SMILES: Cc1cc(C(C)Nc2cccc(F)c2C(=O)O)c2oc(-c3ccc4nn(C)cc4c3)cc(=O)c2c1] | 2-fluoro-6-((1-(6-methyl-2-(2- methyl-2H-indazol-5-yl)-4-oxo- 4H-chromen-8-yl)ethyl) amino)benzoic acid |
| 185 | 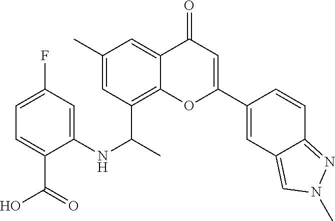 [SMILES: Cc1cc(C(C)Nc2cc(F)ccc2C(=O)O)c2oc(-c3ccc4nn(C)cc4c3)cc(=O)c2c1] | 4-fluoro-2-((1-(6-methyl-2-(2- methyl-2H-indazol-5-yl)-4-oxo- 4H-chromen-8-yl)ethyl) amino)benzoic acid |
| 186 | 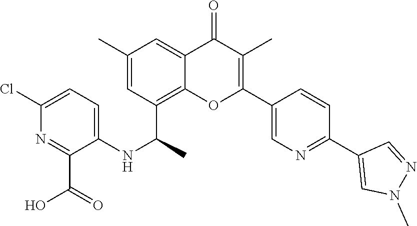 [SMILES: Cc1cc([C@@H](C)Nc2ccc(Cl)nc2C(=O)O)c2oc(-c3ccc(-c4cnn(C)c4)nc3)c(C)c(=O)c2c1] | (R)-6-chloro-3-((1-(3,6-dimethyl-2- (6-(1-methyl-1H-pyrazol-4- yl)pyridin-3-yl)-4-oxo-4H-chromen- 8-yl)ethyl)amino)picolinic acid |
| 187 | 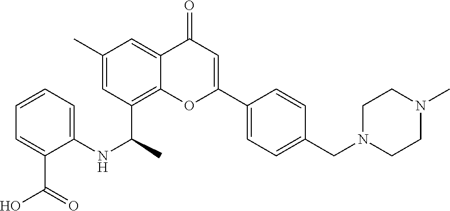 [SMILES: Cc1cc([C@@H](C)Nc2ccccc2C(=O)O)c2oc(-c3ccc(CN4CCN(C)CC4)cc3)cc(=O)c2c1] | (R)-2-((1-(6-methyl-2- (4-((4-methylpiperazin-1- yl)methyl)phenyl)-4- oxo-4H-chromen-8- yl)ethyl)amino)benzoic acid |
| 188 | 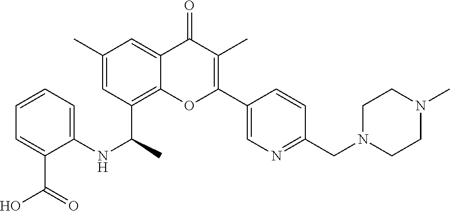 [SMILES: Cc1cc([C@@H](C)Nc2ccccc2C(=O)O)c2oc(-c3ccc(CN4CCN(C)CC4)nc3)c(C)c(=O)c2c1] | (R)-2-((1-(3,6-dimethyl-2- (6-((4-methylpiperazin-1- yl)methyl)pyridin-3-yl)- 4-oxo-4H-chromen-8- yl)ethyl)amino) benzoic acid |
| 189 | 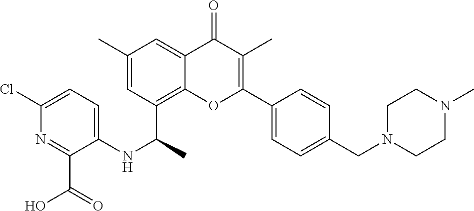 [SMILES: Cc1cc([C@@H](C)Nc2ccc(Cl)nc2C(=O)O)c2oc(-c3ccc(CN4CCN(C)CC4)cc3)c(C)c(=O)c2c1] | (R)-6-chloro-3-((1-(3,6-dimethyl- 2-(4-((4-methylpiperazin-1- yl)methyl)phenyl)-4- oxo-4H-chromen-8- yl)ethyl)amino) picolinic acid |
| 190 | 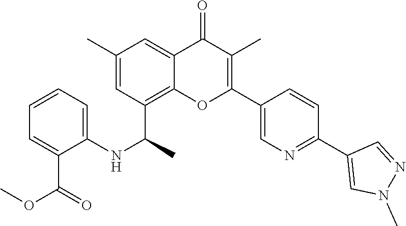 [SMILES: COC(=O)c1ccccc1N[C@H](C)c1cc(C)cc2c(=O)c(C)c(-c3ccc(-c4cnn(C)c4)nc3)oc12] | methyl (R)-2-((1-(3,6-dimethyl- 2-(6-(1-methyl-1H-pyrazol-4- yl)pyridin-3-yl)-4-oxo-4H- chromen-8-yl)ethyl)amino)benzoate |
| 191 | 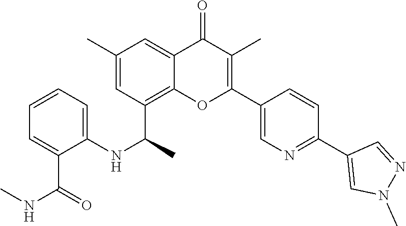 [SMILES: CNC(=O)c1ccccc1N[C@H](C)c1cc(C)cc2c(=O)c(C)c(-c3ccc(-c4cnn(C)c4)nc3)oc12] | (R)-2-((1-(3,6-dimethyl-2-(6- (1-methyl-1H-pyrazol-4- yl)pyridin-3-yl)-4-oxo-4H- chromen-8-yl)ethyl)amino)-N- methylbenzamide |
| 192 | 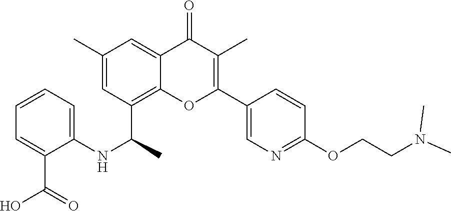 [SMILES: Cc1cc([C@@H](C)Nc2ccccc2C(=O)O)c2oc(-c3ccc(OCCN(C)C)nc3)c(C)c(=O)c2c1] | (R)-2-((1-(2-(6-(2-(dimethylamino) ethoxy)pyridin-3-yl)-3,6- dimethyl-4-oxo-4H-chromen- 8-yl)ethyl)amino)benzoic acid |

In one embodiment, the present invention provides a pharmaceutical composition comprising a compound of Formula (VI), (VII), (VII-1) or (VIII), the stereoisomer or a pharmaceutically acceptable salt, prodrug, or solvate salt thereof and one or more pharmaceutically acceptable carriers or excipients.

In one embodiment, the compounds described herein may be used to treat diseases that are mediated by PI3Kα mutations, or the compounds described herein may be used to prepare a medicament for treating diseases that are mediated by PI3Kα mutations. In certain embodiments, the disease is a hematologic malignancy. In certain embodiments, the disease is lymphoma, such as Burkitt lymphoma, diffuse large B-cell lymphoma (DLBCL) and mantle cell lymphoma (MCL), follicular lymphoma, lynphoplasmacytic lymphonia, Waldenstrom macroglobulinemia, and marginal zone lymphoma. In one embodiment, the disorder is multiple myeloma, or leukemia, such as acute lymphocytic leukemia (ALL), acute myeloid leukemia (AML), chronic lymphocytic leukemia (CLL), small lymphocytic lymphoma (SLL), myelodysplastic syndrome (MDS), myeloproliferative disease (MPD), chronic myeloid leukemia (CML).

In other embodiments, the disease is a solid tumor. In particular embodiments, the indication is to treat solid tumor with PI3Kα mutations, such as pancreatic ductal adenocarcinoma (PDAC) and hepatocellular carcinoma (HCC), gastrointestinal cancer, prostate cancer, ovarian cancer, medulloblastoma, and breast cancer. In some embodiment, the compounds alone or with combination of other anti-cancer therapies may be used to treat prostate cancer, bladder cancer, colorectal cancer, renal cancer, hepatocellular cancer, lung cancer, ovarian cancer, cervical cancer, head and neck cancer, melanoma, neuroendocrine cancers, brain tumors, bone cancer, or soft tissue sarcoma.

In one embodiment, a compound of Formula (VI), (VII), (VII-1) or (VIII), the stereoisomer or a pharmaceutically acceptable salt, prodrug, or solvate may be used in combination with one or more additional therapeutic agents to treat cancers or inflammatory disorders. The one or more additional therapeutic agents may be a chemotherapeutic agent, a radiotherapy, a targeted therapy, an immunotherapeutic agent or any current best of care treatment, either as a small molecule or a biologic nature.

In one embodiment, methods of treating a PI3K mutation disorder comprise administering to a subject in need thereof a compound of Formula (VI), (VII), (VII-1) or (VIII), the stereoisomer or a pharmaceutically acceptable salt, solvate or prodrug thereof, or a pharmaceutical formulation thereof are provided.

### DETAILED DESCRIPTION OF THE INVENTION

The present invention may be understood more readily by reference to the following detailed description of the preferred embodiments of the invention and the Examples included herein. It is to be understood that the terminology used herein is for the purpose of describing specific embodiments only and is not intended to be limiting.

Further, one or more hydrogen atoms, carbon atoms or other atoms of the compound of Formula (VI), (VII), (VII-1) or (VIII), the stereoisomer or a pharmaceutically acceptable salt, prodrug, or solvate salt thereof can be substituted by an isotope of a hydrogen atom, a carbon atom or other atoms, respectively. In addition, the compound of Formula (VI), (VII), (VII-1) or (VIII), the stereoisomer or a pharmaceutically acceptable salt, prodrug, or solvate salt thereof includes all radioactive labeled bodies thereof. Such the “radioactive labeling” and “radioactive labeled form” of the compound of Formula (VI), (VII), (VI-1) or (VIII), the stereoisomer or a pharmaceutically acceptable salt, prodrug, or solvate salt thereof are included in the present invention, respectively, and are useful as a study and/or diagnostic tool in metabolized drug dynamic state study and binding assay. Examples of an isotope which can be incorporated into the compound of the compound of Formula (VI), (VII), (VII-1) or (VIII), the stereoisomer or a pharmaceutically acceptable salt, prodrug, or solvate salt thereof of the present invention include a hydrogen atom, a carbon atom, a nitrogen atom, an oxygen atom, a phosphorus atom, a sulfur atom, a fluorine atom and a chlorine atom, such as ²H, ³H, ¹³C, ¹⁴C, N, ¹⁸O, ¹⁷O, ³¹P, ²P, ³⁵S, ¹⁸F, and ³⁶Cl. A particularly preferable example of an isotope which can be incorporated into the compound of Formula (VI), (VII), (VII-1) or (VIII), the stereoisomer or a pharmaceutically acceptable salt, prodrug, or solvate salt thereof of the present invention is ²H (i.e., heavy hydrogen atom), and can be prepared by the method shown in Reference examples of the present description, or the method well-known in the art. In addition, a heavy hydrogen atom is expressed as “D” in Reference examples of the present description. The compound of Formula (VI), (VII), (VII-1) or (VIII), the stereoisomer or a pharmaceutically acceptable salt, prodrug, or solvate salt thereof of the present invention in which a hydrogen atom has been converted into a heavy hydrogen atom are excellent in respect of bioavailability, metabolism safety, drug efficacy, and toxicity as compared with unconverted forms, in some cases, and can be useful as medicaments.

As used herein, the term “n-membered” where n is an integer typically describes the number of ring-forming atoms in a moiety where the number of ring-forming atoms is n. For example, pyridine is an example of a 6-membered heteroaryl ring and thiophene is an example of a 5-membered heteroaryl ring.

The term “alkyl” refers to a saturated aliphatic hydrocarbon group, which is a straight or branched chain group comprising 1 to 20 carbon atoms, preferably an alkyl having 1 to 8 carbon atoms, more preferably an alkyl having 1 to 6 carbon atoms (C₁-C₆ alkyl), and most preferably an alkyl having 1 to 3 carbon atoms. Non-limiting examples include methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, I-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylbexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and various branched isomers thereof. For example, the term “C₁₋₄alkyl” means an alkyl group having 1 to 4 carbon atoms. Specifically, C₁₋₄ alkyl is intended to include C₁ alkyl (methyl), C₂ alkyl (ethyl), C₃ alkyl (n-propyl, isopropyl), C₄ alkyl (i.e., n-butyl, t-butyl, isobutyl, sec-butyl). The alkyl group can be substituted or unsubstituted. When substituted, the substituent group(s) can be substituted at any available connection point. The substituent group(s) is preferably one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocyclyloxy, cycloalkylthio, heterocyclylthio, oxo, carboxy and carboxylate group, and preferably methyl, ethyl, isopropyl, tert-butyl, haloalkyl, deuterated alkyl, alkoxy-substituted alkyl and hydroxy-substituted alkyl.

The term “alkenyl” as used herein refers to an unsaturated linear (i.e., unbranched) or branched univalent hydrocarbon chain or combination thereof, having at least one site of olefinic unsaturation (i.e., having at least one moiety of the formula a C═C) and having the number of carbon atoms designated. The alkenyl group may be in “cis” or “trans” configurations, or alternatively in “E” or “Z” configurations. The alkenyl is preferably an alkenyl having 2 to 8 carbon atoms, more preferably an alkenyl having 2 to 6 carbon atoms (C₂-C₆ alkenyl), and most preferably an alkenyl having 2 to 3 carbon atoms. The alkenyl can be further substituted by other related group, for example alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocyclyloxy, cycloalkylthio, heterocyclylthio, carboxy or carboxylate group.

The term “alkynyl” as used herein refers to an unsaturated linear (i.e., unbranched) or branched univalent hydrocarbon chain or combination thereof, having at least one site of acetylenic unsaturation (i.e., having at least one moiety of the formula C≡C) having the number of carbon atoms designated. The alkynyl is preferably an alkynyl having 2 to 8 carbon atoms, more preferably an alkynyl having 2 to 6 carbon atoms (C₂-C₆ alkenyl), and most preferably an alkynyl having 2 to 3 carbon atoms. The alkynyl can be further substituted by other related group, for example alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocyclyloxy, cycloalkylthio, heterocyclylthio, carboxy or carboxylate group.

The term “cycloalkyl” refers to a saturated or partially unsaturated monocyclic or polycyclic hydrocarbon substituent group having 3 to 20 carbon atoms, preferably 3 to 8 carbon atoms (i.e., (C₃₋₈ cycloalkyl means a cycloalkyl with three to eight carbon atoms), and more preferably 3 to 6 carbon atoms. Non-limiting examples of monocyclic cycloalkyl include cyclopropyl, cyclobutyl, cyclopentyl, cyclopentenyl, cyclohexyl, cyclohexenyl, cyclohexadienyl, cycloheptyl, cycloheptatrienyl, cyclooctyl and the like. Polycyclic cycloalkyl includes a cycloalkyl having a spiro ring, fused ring or bridged ring. The cycloalkyl is preferably cyclopropyl, cyclobutyl, cyclohexyl, cyclopentyl and cycloheptyl. The cycloalkyl can be further substituted by other related group, for example alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocyclyloxy, cycloalkylthio, heterocyclylthio, carboxy or carboxylate group.

The term “aryl” as used herein refers to an unsaturated aromatic carbocyclic group having a single ring (e.g., phenyl) or multiple condensed rings (e.g., naphthyl or anthryl) which condensed rings may or may not be aromatic. Particular C₆₋₁₀ aryl groups are those having from 6 to 10 annular (i.e., ring) carbon atoms (for example, phenyl and naphthyl). An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position. Non-limiting examples thereof include:

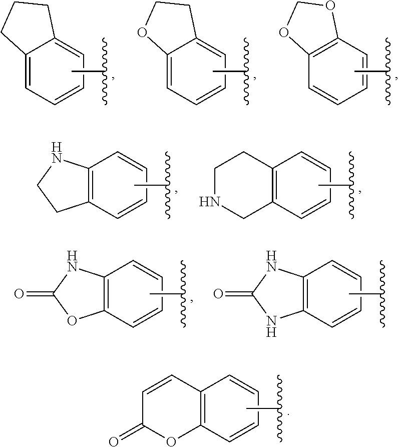

The aryl group can be substituted or unsubstituted. When substituted, the substituent group(s) is preferably one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocyclyloxy, cycloalkylthio, heterocyclylthio, carboxy and carboxylate group.

The term “heteroaryl” as used herein refers to an unsaturated aromatic cyclic group having annular (i.e., ring) carbon atoms and at least one annular heteroatom, including but not limited to heteroatoms such as nitrogen, phosphorus, oxygen and sulfur. A heteroaryl group may have a single ring (e.g., pyridyl, furyl) or multiple condensed rings (e.g., indolizinyl, benzothienyl) which condensed rings may or may not be aromatic. In some embodiments, the heteroatoms disclosed herein are selected from O, S and N. And the term “a 5- to 10-membered heteroaryl” is specifically intended to include any 5-, 6-, 7-, 8-, 9-, or 10-membered heteroaryl group. The heteroaryl is more preferably 5 or 6 membered heteroaryl, for example, imidazolyl, thienyl, thiazolyl, pyrazolyl, oxazolyl, pyrrolyl, triazolyl, tetrazolyl, pyridinyl, pyrimidinyl, thiadiazolyl, pyrazinyl and the like, preferably triazolyl, thienyl, imidazolyl, pyrazolyl or pyrimidinyl, thiazolyl; and more preferably triazolyl, pyrrolyl, thienyl, thiazolyl and pyrimidinyl. Non-limiting examples thereof include:

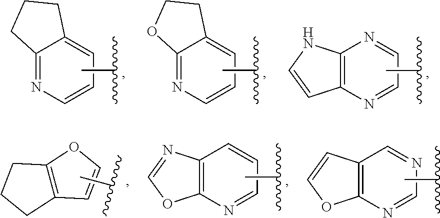

The heteroaryl group can be optionally substituted or unsubstituted. When substituted, the substituent group(s) is preferably one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocyclyloxy, cycloalkylthio, heterocyclylthio, carboxy and carboxylate group.

The term “heterocyclyl” refers to a 3 to 20 membered saturated or partially unsaturated monocyclic or polycyclic hydrocarbon substituent group, wherein one or more ring atoms are heteroatoms selected from the group consisting of N, O and S(O)m (wherein m is an integer of 0 to 2), but excluding —O—O—, —O—S— or —S—S— in the ring, with the remaining ring atoms being carbon atoms. Preferably, the heterocyclyl has 3 to 12 ring atoms wherein 1 to 4 atoms are heteroatoms; more preferably, the heterocyclyl has 4 to 10 ring atoms; and most preferably 4 to 7 ring atoms. For example, the term “4- to 10-membered heterocyclyl” means the heterocyclyl having 4 to 10 ring atoms. Non-limiting examples of monocyclic heterocyclyl include pyrrolidinyl, imidazolidinyl, tetrahydrofuranyl, tetrahydrothienyl, dihydroimidazolyl, dihydrofuryl, dihydropyrazolyl, dihydropyrrolyl, piperidinyl, piperazinyl, morpholinyl, thiomorpholinyl, homopiperazinyl, pyranyl and the like, and preferably tetrahydrofuranyl, and tetrahydropyranyl, pyrazolyl, morpholinyl, piperazinyl and pyranyl. Polycyclic heterocyclyl includes a heterocyclyl having a spiro ring, fused ring or bridged ring. The heterocyclyl having a spiro ring, fused ring or bridged ring is optionally bonded to another group via a single bond, or further bonded to other cycloalkyl, heterocyclyl, aryl and heteroaryl via any two or more atoms on the ring. The heterocyclyl group can be optionally substituted or unsubstituted. When substituted, the substituent group(s) is preferably one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocyclyloxy, cycloalkylthio, heterocyclylthio, oxo, carboxy and carboxylate group.

The term “halo” or “halogen” refers to fluorine, chlorine, bromine or iodine.

The term “haloalkyl” refers to an alkyl group substituted with one or more halogen that may be the same of different, wherein the alkyl is as defined above. For example, “C₁₋₆ haloalkyl” as used herein, refers to a C₁0.6 alkyl group wherein the alkyl group is substituted with one or more halogen atoms. A C₁₋₆ haloalkyl may be selected from fluromethyl, fluoroethyl, difluoromethyl, difluoroethyl, trifluoromethyl, trifluoroethyl, 1,1-difluoroethyl.

The term “alkoxy” refers to an —O-(alkyl) or an —O— (unsubstituted cycloalkyl) group, wherein the alkyl is as defined above. The alkoxy is preferably an alkoxy having 1 to 8 carbon atoms, more preferably an alkoxy having 1 to 6 carbon atoms (C₁-C₆ alkoxy), and most preferably an alkoxy having 1 to 3 carbon atoms. Non-limiting examnples of alkoxy include mnethoxy, ethoxy, propoxy, butoxy, cyclopropyloxy, cyclobutyloxy, cyclopentyloxy, cyclohexyloxy. The alkoxy group can be optionally substituted or unsubstituted. When substituted, the substituent group(s) is preferably one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocyclyloxy, cycloalkylthio, heterocyclylthio, carboxy and carboxylate group.

The term “haloalkoxy” refers to an alkoxy group substituted by one or more halogens, wherein the alkoxy is as defined above.

The term “deuterium” refers to an isotope of hydrogen that has one proton and one neutron in its nucleus and that has twice the mass of ordinary hydrogen, the symbol of deuterium is “D”.

The term “deuterated alkyl” refers to an alkoxy group substituted by one or more deuterium, wherein the alkyl is as defined above.

The term “hydroxyalkyl” refers to an alkoxy group substituted by one or more hydroxy, wherein the alkyl is as defined above.

The term “alkylthio” refers to “—S-alkyl”, wherein the alkyl is as defined above. The term “—C(O)NH-alkyl” or “—NH C(O)-alkyl” refers to an “—C(O)NH-” group connected with an alkyl, wherein the alkyl is as defined above. And “methylcarbamoyl” refers to —C(O)NH-methyl.

The term “ester group” refers to an “—C(O)O—” group connected with an alkyl, wherein the alkyl is as defined above.

The term “amino” refers to a —NH₂ group.

The term “—NH (C₁-Cₑ alkyl)” or “—N(C₁-C₆ alkyl)₂” refers to an amino substituted by one or two C₁-C₆ alkyl.

The term“nitro” refers to a —NO₂ group.

The term“hydroxy” refers to an —OH group.

The term “thiol” refers to an —S— group.

The term“cyano” refers to a —CN group.

The term “Optionally substituted”, as used herein, means that substitution is optional and therefore includes both unsubstituted and substituted atoms and moieties. A “substituted” atom or moiety indicates that any hydrogen on the designated atom or moiety can be replaced with a selection from the indicated substituent group (up to and including that every hydrogen atom on the designated atom or moiety is replaced with a selection from the indicated substituent group), provided that the normal valency of the designated atom or moiety is not exceeded, and that the substitution results in a stable compound. For example, if a methyl group (i.e., —CH₃) is optionally substituted, then up to 3 hydrogen atoms on the carbon atom can be replaced with substituent groups.

A salt or “a Pharmaceutically acceptable salt” means a salt prepared by conventional means, and are well known by those skilled in the art. The “pharmacologically acceptable salts” include basic salts of inorganic and organic acids (Berge et al., J Pharm. Sci. 1977, 66:1).

A “solvate” is formed by treating a compound in a solvent. Solvates of salts of the compounds are also provided. In the case of treating compounds with water, the solvate is hydrates.

A “prodrug” includes any compound that converts into a compound of the present invention, when administered to a subject, e.g., upon metabolic processing of the prodrug.

The term “stereoisomer” refers to compounds which have identical chemical constitution, but differ with regard to the arrangement of the atoms or groups in space.

As used herein, “treat” or “treating” in reference to a disorder means to ameliorate or prevent the disorder or one or more of the biological manifestations of the disorder, to interfere with one or more points in the biological cascade that leads to or is responsible for the disorder, to alleviate one or more of the symptoms or effects associated with the disorder. As indicated above, “treatment” of a disorder includes prevention of the disorder, and “prevention” is understood to refer to the prophylactic administration of a drug to substantially diminish the likelihood or severity of a disorder or biological manifestation thereof, or to delay the onset of such disorder or biological manifestation thereof.

The term “Subject” refers to a human (including adults and children) or other animals. In one embodiment, “patient” refers to a human.

As used herein, “safe and effective dose” in reference to a compound of formulas, or a pharmaceutically acceptable salt, prodrug, or solvate thereof an amount sufficient to treat the patient's condition but low enough to avoid serious side effects. A safe and effective dose of a compound will vary with the particular compound chosen (e.g. consider the potency, efficacy, and half-life of the compound); the route of administration chosen; the disorder being treated; the severity of the disorder being treated; the age, size, weight, and physical condition of the patient being treated; the medical history of the patient to be treated; the duration of the treatment; the nature of concurrent therapy; the desired therapeutic effect; and like factors.

The potencies of compounds as inhibitors of an enzyme activity (or other biological activity) can be established by determining the concentrations at which each compound inhibits the activity to a predefined extent and then comparing the results. “IC₅₀” or “IC₉₀” of an inhibitor can be determined by the concentration that inhibits 50% or 90% of the activity in a biochemical assay, which can be accomplished using conventional techniques known in the art, including the techniques describes in the examples below.

### PREFERRED EMBODIMENTS

The present invention is further described in combination with the following examples, which are not intended to limit the scope of the present invention.

### EXAMPLES

The compounds of the present invention may be prepared using the methods disclosed herein and routine modifications thereof, which will be apparent given the disclosure herein and methods are well known in the art. Conventional and well-known synthetic methods may be used in addition to the teachings herein. The synthesis of representative compounds described herein may be accomplished as described in the following examples. If available, reagents may be purchased commercially, e.g., from Sigma Aldrich or other chemical suppliers. The starting materials used in the examples of the present invention are known and commercially available, or can be synthesized by adopting or according to known methods in the art.

### General

The structures of the compounds of the present invention were identified by nuclear magnetic resonance (NMR) and/or liquid chromatography-mass spectrometry (LC-MS). II-NMR spectra are recorded on a Bruker 400 MHZ NMR spectrometer. Significant peaks are tabulated in the order: multiplicity (s, singlet; d, doublet; t, triplet; q, quartet; m, multiplet; br s, broad singlet), coupling constant(s) in Hertz (Hz) and number of protons. Mass spectrometry results are reported as the ratio of mass over charge, followed by the relative abundance of each ion (in parentheses Electrospray ionization (ESI) mass spectrometry analysis is conducted on a Shimadzu LC/MSD electrospray mass spectrometer.

### Synthetic Reaction

The terms “solvent”, “inert organic solvent”, or “inert solvent” refer to a solvent inert under the conditions of the reaction being described in conjunction therewith (including, for example, benzene, toluene (“Tol”), acetonitrile, tetrahydrofuran (“THF”), diethylformamide (“DMF”), ethyl acetate (EA or EtOAc), dichloromethane (DCM), diethyl ether, methanol, pyridine and the like. Unless specified to the contrary, the solvents used in the reactions of the present invention are inert organic solvents, and the reactions are carried out under an inert gas, preferably nitrogen and argon.

“SFC” refers to Supercritical Fluid Chromatography, the stereoisomers of example compounds can be prepared with chiral SFC (column: DAICEL CHIRALCEL OD (250 mm*30 mm, 10 um).

“DIEA” or “DIPEA” refers to N, N-Diisopropylethylamine.

“Pd(dba)₂” refers to “Tris(dibenzylideneacetone)dipalladium”

“X-phos” refers to Chloro (2-dicyclohexylphosphino-2′,4′,6′-tri-i-propyl-1,1′-biphenyl) (2′-amino-1,1′-biphenyl-2-yl) palladium (II).

“TFA” refers to Trifluoroacetic acid.

“LHMDS” refers to Lithium bis(trimethylsilyl)amide.

“m-CPBA” refers to m-ChloroperbenzoicAcid.

“Pd (PPh₃)₄” refer to “Tetrakis(triphenylphosphine)palladium”.

“Pd (dppf)₂Cl₂” refers to [1,1′-Bis(diphenylphosphino)ferrocene] dichloropalladium(II).

“STAB” refers to “Sodium Triacetoxyborohydride”.

“Tf₂O” refers to “Trifluoromethanesulfonic anhydride”.

“EDC” refers to “1-Ethyl-3-(3-dimethylaminopropyl) carbodiimide”.

“MTBE” refers to “Methyl tert-butyl ether”.

“Pd (PPh₃)₂Cl₂” refers to “Bis(triphenylphosphine) palladium (II) chloride”.

“NMP” refers to “N-methylpyrrolidone”.

“NBS” refers to “N-Bromosuccinimide”.

“HATU” refers to “2-(7-Azabenzotriazol-1-yl)-N, N, N′, N′-tetramethyluronium hexafluorophosphate”.

“CuTC” refers to “thiophene-2-carbonyloxycopper”.

“B(PIN)₂” refers to “Bis(pinacolato)diboron”.

### Example 1

### 2-((1-(6-methyl-4-oxo-2-phenyl-4H-chromen-8-yl)ethyl)amino)benzoic acid

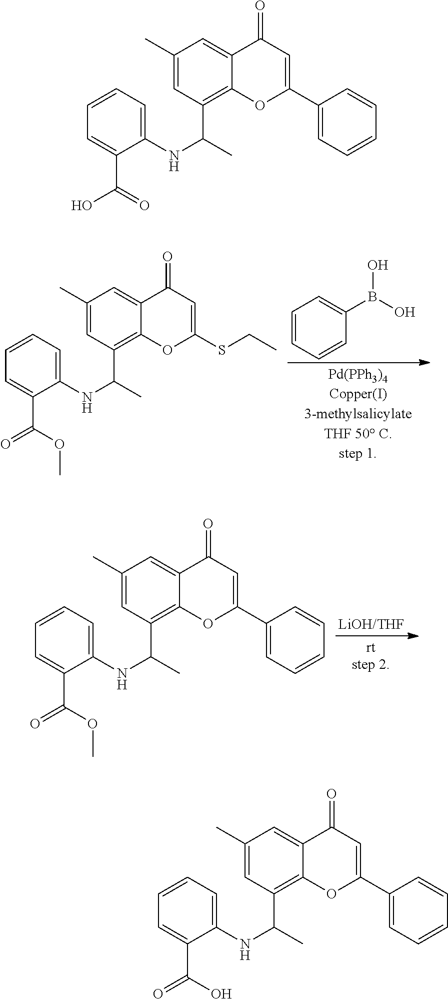

Methyl 2-((1-(2-(ethylthio)-6-methyl-4-oxo-4H-chromen-8-yl)ethyl)amino)benzoate is prepared in accordance with the method in WO2021202964A1

### Step 1

To a solution of methyl 2-[1-(2-ethylsulfanyl-6-methyl-4-oxo-chromen-8-yl)ethylamino]-benzoate (30 mg, 75.47 μmol) and phenylboronic acid (11.96 mg, 98.12 μmol) in dioxane (1 mL) was added CuI (28.75 mg, 150.95 μmol), Pd(dppf)Cl₂ (11.04 mg. 15.09 μmol) and Cs₂CO₃ (49.18 mg, 150.95 μmol) under the N₂ at 15° C. The mixture was stirred at 80° C. for 14 h under N₂. The reaction mixture was filtered and concentrated under reduced pressure to give a residue. The residue was combined with ES21039-11 and then purified by flash silica gel chromatography to give methyl 2-[1-(6-methyl-4-oxo-2-phenyl-chromen-8-yl)ethylamino]benzoate (58 mg, 117.83 unol, 78.1% yield, 84% purity) as yellow oil.

MS m/z (ESI): 414 [M+H]⁺.

### Step 2

To a solution of methyl 2-[1-(6-methyl-4-oxo-2-phenyl-chromen-8-yl)ethylamino]benzoate (58 mg, 140.28 μmol) in MeOH (1 mL) and H₂O (1 mL) was added NaOH (6.17 mg, 154.31 μmol) at 15° C., and the mixture was stirred for 1 h at 15° C. The reaction mixture was concentrated under reduced pressure to give a residue. The residue was purified by prep-HPLC (column: Welch Xtimate C18 150*30 mm*5 um; mobile phase: [water (FA)-ACN]; B %: 52%-82%, 7 min.) to give 2-[1-(6-methyl-4-oxo-2-phenyl-chromen-8-yl) ethylamino]benzoic acid (13.6 mg, 34.05 μmol, 24.27% yield, 100% purity) as brown solid. MS m/z (ESI): 400 [M+H]⁺.

¹H NMR (400 MHz, DMSO-d₆) δ ppm 8.56 (br s, 1H), 8.14 (d, J=6.0 Hz, 1H), 7.82 (d, J=8.0 Hz, 1H), 7.75 (s, 1H), 7.61-7.56 (m, 4H), 7.22-7.18 (m, 1H), 7.09 (s, 1H), 6.56-6.49 (m, 2H), 5.35-5.32 (m, 1H), 2.36 (s, 3H), 1.67 (d, 7=6.4 Hz, 3H).

### Example 2

### 2-((1-(6-methy-4-oxo-2-(p-tolyl)-4H-chromen-8-yl)ethyl)amino)benzoic acid

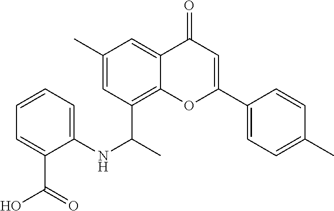 [SMILES: Cc1ccc(-c2cc(=O)c3cc(C)cc(C(C)Nc4ccccc4C(=O)O)c3o2)cc1]

2-((1-(6-methyl-4-oxo-2-(p-tolyl)-4H-chromen-8-yl)ethyl)amino)benzoic acid was prepared in accordance with the method of Example 1.

MS m/z (ESI): 414 [M+H]⁺.

¹H NMR (400 MHz, DMSO-d6) δ ppm 12.45-13.04 (m, 1H) 8.43 (br d, J=6.00 Hz, 1H) 8.02 (d, J=8.26 Hz, 2H) 7.82 (dd, J=7.94, 1.31 Hz, 1H) 7.75 (s, 1H) 7.56 (d, J=1.75 Hz, 1H) 7.40 (d, J==8.00 Hz, 2H) 7.17-7.31 (n, 1H) 7.03 (s, 1H) 6.47-6.61 (m, 2H) 5.33 (br t, J=6.38 Hz, 1H) 2.41 (s, 3H) 2.37 (s, 3H) 1.68 (d, J=6.63 Hz, 3H).

### Example 3

### 2-((1-(2-(4-chlorophenyl)-6-methyl-4-oxo-4H-chromen-8-yl)ethyl)amino)benzoic acid

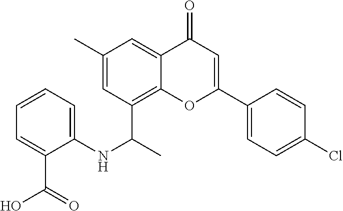 [SMILES: Cc1cc(C(C)Nc2ccccc2C(=O)O)c2oc(-c3ccc(Cl)cc3)cc(=O)c2c1]

2-((1-(2-(4-chlorophenyl)-6-methyl-4-oxo-4H-chromen-8-yl)ethyl)amino)benzoic acid was prepared in accordance with the method of Example 1.

MS: m/z 434.2 [M+1H];

¹H NMR (400 MHz, CD3OD-d4) δ ppm 8.07 (d, J=8.4 Hz, 1H), 7.92 (d, J=8.0 Hz, 1H), 7.85 (s, 1H), 7.64-7.59 (m, 3H), 7.21-7.17 (m, 1H), 6.97 (s, 1H), 6.58-6.48 (m, 2H), 5.37-5.32 (m, 1H), 2.41 (s, 3H), 1.74 (d, J=6.4 Hz, 3H).

### Example 4

### 2-((1-(2-(benzo[d][1,3]dioxol-5-yl)-6-methyl-4-oxo-4H-chromen-8-yl)ethyl)amino)benzoic acid

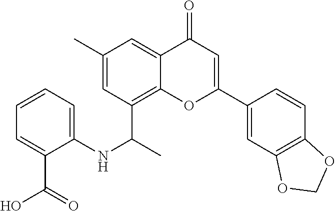 [SMILES: Cc1cc(C(C)Nc2ccccc2C(=O)O)c2oc(-c3ccc4c(c3)OCO4)cc(=O)c2c1]

2-((1-(2-(benzo[d][1,3]dioxol-5-yl)-6-methyl-4-oxo-4H-chromen-8-yl)ethyl)amino)benzoic acid was prepared in accordance with the method of Example 1

MS: m/z 444.2 [M+H]⁺;

¹H NMR (400 MHz, DMSO)-d6) δ ppm 8.58 (br s, 1H), 7.81 (d, J=8.0 Hz, 1H), 7.72-7.69 (m, 3H), 7.53 (s, 1H), 7.21-7.17 (m, 1H), 7.11 (d, J=8.0 Hz, 1H), 7.00 (s, 1H), 6.55-6.46 (in. 2H), 6.16 (s, 2H), 5.31 (br s, 1H), 2.35 (s, 3H), 1.65 (d, J=6.8 Hz, 3H).

### Example 5

### 2-((1-(6-methyl-4-oxo-2-(1-oxoisoindolin-5-yl)-4H-chromen-8-yl)ethyl)amino)benzoic acid

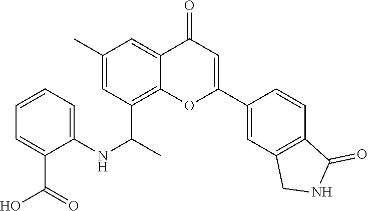 [SMILES: Cc1cc(C(C)Nc2ccccc2C(=O)O)c2oc(-c3ccc4c(c3)CNC4=O)cc(=O)c2c1]

2-((1-(6-methyl-4-oxo-2-(1-oxoisoindolin-5-yl)-4H-chromen-8-yl)ethyl)amino)benzoic acid was prepared in accordance with the method of Example 1.

MS: m/z 455.2 [M+H]⁺;

¹H NMR (400 MHz, DMSO-de) δ ppm 8.79 (s, 1H), 8.48 (br s, 1H), 8.33 (s, 1H), 8.23 (d, J=8.0 Hz, 1H), 7.82 (d, J=8.0 Hz, 1H), 7.76 (d, J=1.2 Hz, 1H), 7.60 (d, J=1.6 Hz, 1H), 7.25-7.21 (m, 1H), 7.16 (s, 1H), 6.58-6.53 (m, 2H), 5.37-5.34 (m, 2H), 2.38 (s, 3H), 1.68 (d, J=6.8 Hz, 3H).

### Example 6

### 2-((1-(6-methyl-2-(2-methyl-1-oxoisoindolin-5-yl)-4-oxo-4H-chromen-8-yl)ethyl)amino)benzoic acid

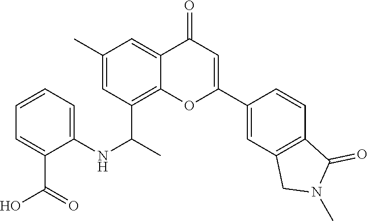 [SMILES: Cc1cc(C(C)Nc2ccccc2C(=O)O)c2oc(-c3ccc4c(c3)CN(C)C4=O)cc(=O)c2c1]

2-((1-(6-methyl-2-(2-methyl-1-oxoisoindolin-5-yl)-4-oxo-4H-chromen-8-yl)ethyl)amino)benzoic acid was prepared in accordance with the method of Example 1. MS: m/z 469.2 [M+H]⁺;

¹H NMR (400 MHz, DMSO-d6) δ ppm 8.46 (br s, 1H), 8.34 (s, 1H), 8.22 (d, J=8.0 Hz, 1H), 7.83-7.80 (n, 2H), 7.76 (s, 1H) 7.60 (d, J=1.6 Hz, 1H), 7.25-7.22 (m, 1H), 7.16 (s, 1H), 6.58-6.53 (m, 2H), 5.38-5.34 (m, 2H), 4.56 (s, 2H), 3.11 (s, 3H), 2.38 (s, 3H), 1.68 (d, J=6.8 Hz, 3H).

### Example 7

### 2-((1-(2-(2-ethyl-1-oxoisoindolin-5-yl)-6-methyl-4-oxo-4H-chromen-8-yl)ethyl)amino)benzoic acid

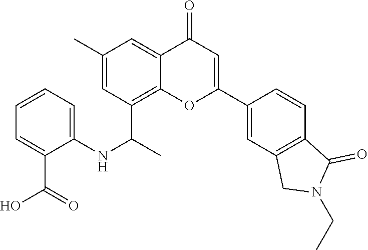 [SMILES: CCN1Cc2cc(-c3cc(=O)c4cc(C)cc(C(C)Nc5ccccc5C(=O)O)c4o3)ccc2C1=O]

2-((1-(2-(2-ethyl-1-oxoisoindolin-5-yl)-6-methyl-4-oxo-4H-chromen-8-yl)ethyl)amino)benzoic acid was prepared in accordance with the method of Example 1.

¹H NMR (400 MHz, DMSO-d₆) δ ppm 8.53 (br s, 1H), 8.36 (s, 1H), 8.24 (br d, J=7.78 Hz, 1H), 7.74-7.85 (m, 3H), 7.60 (s, 1H), 7.15-7.26 (m, 2H), 6.51-6.59 (m, 2H), 5.36 (br s, 1H), 4.59 (s, 2H), 3.56-3.61 (m, 2H), 2.38 (s, 3H), 1.68 (br d, J=6.53 Hz. 3H), 1.21 (br t, J=7.15 Hz, 3H).

MS m/z [M+H]⁺: 483.1

### Example 8

### 2-((1-(2-(2-cyclopropyl-1-oxoisoindolin-5-yl)-6-methyl-4-oxo-4H-chromen-8-yl)ethyl)amino)benzoic acid

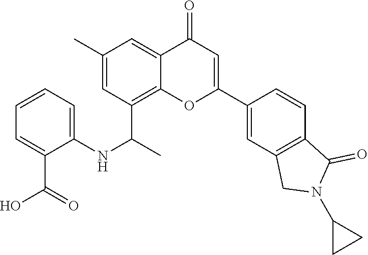 [SMILES: Cc1cc(C(C)Nc2ccccc2C(=O)O)c2oc(-c3ccc4c(c3)CN(C3CC3)C4=O)cc(=O)c2c1]

2-((1-(2-(2-cyclopropyl-1-oxoisoindolin-5-yl)-6-methyl-4-oxo-4H-chromen-8-yl)ethyl)amino)benzoic acid is prepared in accordance with the method of Example 1.

MS m/z (ESI): 495 [M+H]⁺.

### Example 9

### 2-((1-(6-methyl-4-oxo-2-(1-oxo-1,2,3,4-tetrahydroisoquinolin-6-yl)-4H-chromen-8-yl)ethyl)amino)benzoic acid

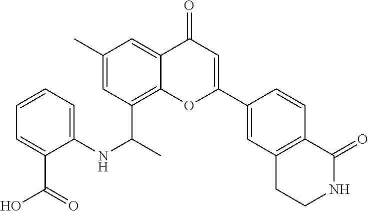 [SMILES: Cc1cc(C(C)Nc2ccccc2C(=O)O)c2oc(-c3ccc4c(c3)CCNC4=O)cc(=O)c2c1]

2-((1-(6-methyl-4-oxo-2-(1-oxo-1,2,3,4-tetrahydroisoquinolin-6-yl)-4H-chromen-8-yl)ethyl)amino)benzoic acid is prepared in accordance with the method of Example 1.

MS m/z (ESI): 469 [M+H]⁺.

### Example 10

### 2-((1-(6-methyl-2-(1-methyl-1H-pyrazol-4-yl)-4-oxo-4H-chromen-8-yl)ethyl)amino)benzoic acid

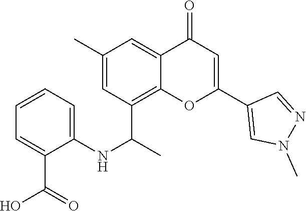 [SMILES: Cc1cc(C(C)Nc2ccccc2C(=O)O)c2oc(-c3cnn(C)c3)cc(=O)c2c1]

2-((1-(6-methyl-2-(1-methyl-1H-pyrazol-4-yl)-4-oxo-4H-chromen-8-yl)ethyl)amino)benzoic acid was prepared in accordance with the method of Example 1.

MS: m/z (ESI): 404.2 [M+H]⁺;

¹H NMR (400 MHz, DMSO-d6) δ ppm 8.56 (s, 1H), 8.42 (br s, 1H), 8.20 (s, 1H), 7.82-7.80 (m, 1H), 7.70 (s, 1H), 7.51 (d, J=2.0 Hz, 1H), 7.25-7.21 (m, 1H), 6.74 (s, 1H), 6.57-6.50 (m, 2H), 5.32-5.30 (m, 1H), 3.93 (s, 1H), 2.34 (s, 3H), 1.63 (d, J=6.4 Hz, 3H).

### Example 11

### 2-((1-(2-(1-(2-hydroxy-2-methylpropyl)-1H-pyrazol-4-yl)-6-methyl-4-oxo-4H-chromen-8-yl)ethyl)amino)benzoic acid

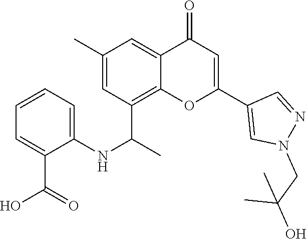 [SMILES: Cc1cc(C(C)Nc2ccccc2C(=O)O)c2oc(-c3cnn(CC(C)(C)O)c3)cc(=O)c2c1]

2-((1-(2-(1-(2-hydroxy-2-methylpropyl)-1H-pyrazol-4-yl)-6-methyl-4-oxo-4H-chromen-8-yl)ethyl)amino)benzoic acid was prepared in accordance with the method of Example 1.

MS m/z (ESI): 462 [M+H]⁺.

¹H NMR (400 MHz, DMSO-d₆) δ ppm 12.78 (br s, 1H) 8.49 (s, 1H) 8.42 (br d, J=6.38 Hz, 1H) 8.21 (s, 1H) 7.81 (dd, J=⁷0.94, 1.31 Hz, 1H) 7.70 (s, 1H) 7.50 (d, J=1.75 Hz, 1H) 7.19-7.27 (m, 1H) 6.77 (s, 1H) 6.55 (t, J=744 Hz, 1H) 6.50 (d, J=0.50 Hz, 1H) 5.30 (br t, J=6.44 Liz, 1H) 4.78 (br s, 1H) 4.10 (s, 2H) 2.34 (s, 3H) 1.64 (d, J=6.63 Hz, 3H) 1.11 (d, J=1.88 Hz, 6H)

### Example 12

### 2-((1-(2-(4,4-dimethylpiperidin-1-yl)-7-(1,1-dioxidothiomorpholine-4-carbonyl)-4-oxo-4H-pyrido[1,2-a]pyrimidin-9-yl)ethyl)amino)benzamide

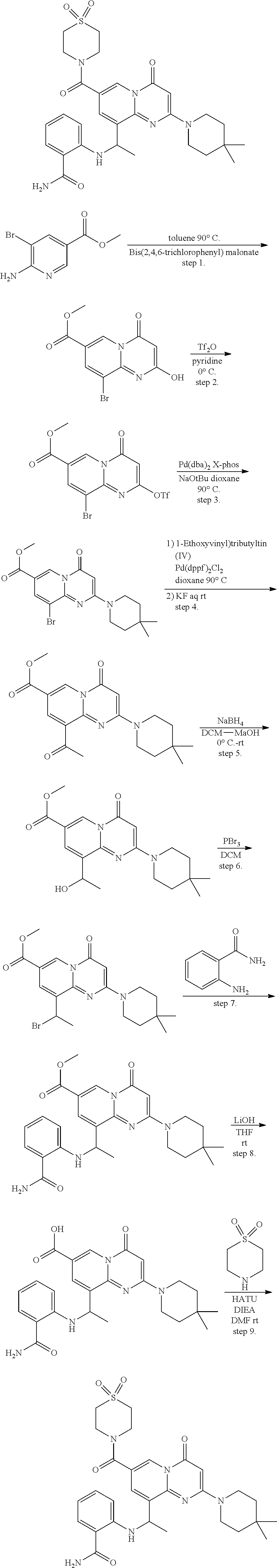

### Step 1

To a solution of methyl 6-amino-5-bromonicotinate (2.29 g, 10.0 mmol) in toluene is added Bis(2,4,6-trichlorophenyl) malonate (10.16 g, 22.0 mmol). The mixture is heated to 90° C. for 18 h. The mixture is cooled down to rt. And the mixture is mixed with silica gel and evaporated. The product/silica gel mixture is placed at the top of a flash chromatography column and eluted to give the methyl 9-bromo-2-hydroxy-4-oxo-4H-pyrido[1,2-a]pyrimidine-7-carboxylate.

MS m/z (ESI): 299 [M+H]⁺.

Steps 2-7 used the methods as described for methyl 2-((1-(2-(ethylthio)-6-methyl-4-oxo-4H-chromen-8-yl)ethyl)amino)benzoate WO2021202964A1.

### Step 8

To a solution of methyl 9-(1-((2-carbamoylphenyl)amino)ethyl)-2-(4,4-dimethylpiperidin-1-yl)-4-oxo-4H-pyrido[1,2-a]pyrimidine-7-carboxylate (477 mg, 1.0 mmol) in THF/H₂O (10 mL/5 mL) is added LiOH (72.0 mg, 3.0 mmol), the mixture is stirred at rt for 2 h. The mixture is concentrated, and acidified with 10% aq HCl, white solid precipitated. And filtration and drying to get the 9-(1-((2-carbamoylphenyl)amino)ethyl)-2-(4,4-dimethylpiperidin-1-yl)-4-oxo-4H-pyrido[1,2-a]pyrimidine-7-carboxylic acid.

MS m/z (ESI: 464 [M+H]⁺.

### Step 9

To a solution of 9-(1-((2-carbamoylphenyl)amino)ethyl)-2-(4,4-dimethylpiperidin-1-yl)-4-oxo-4H-pyrido[1,2-a]pyrimidine-7-carboxylic acid (232.0 mg, 0.5 mmol) in DMF (25 mL) is added thiomorpholine 1,1-dioxide (135 mg, 1.0 mmol) and HATU (200 mg, 0.52 mmol), the mixture is stirred at rt for 4 h. The reaction is quenched by 50 mL water, and extracted with EA for 3 times. The organic phase is combined and washed with brine. After drying (MgSO4) the solvent is evaporated and the residue is purified by column chromatography on silica gel to give 2-((1-(2-(4,4-dimethylpiperidin-1-yl)-7-(1,1-dioxidothiomorpholine-4-carbonyl)-4-oxo-4H-pyrido[1,2-a]pyrimidin-9-yl)ethyl)amino)benzamide.

MS m/z (ESI): 581 [M+H]⁺.

### Example 13

### 2-((1-(2-(1-(tert-butoxycarbonyl)piperidin-4-yl)-6-methyl-4-oxo-4H-chromen-8-yl)ethyl)amino)benzoic acid

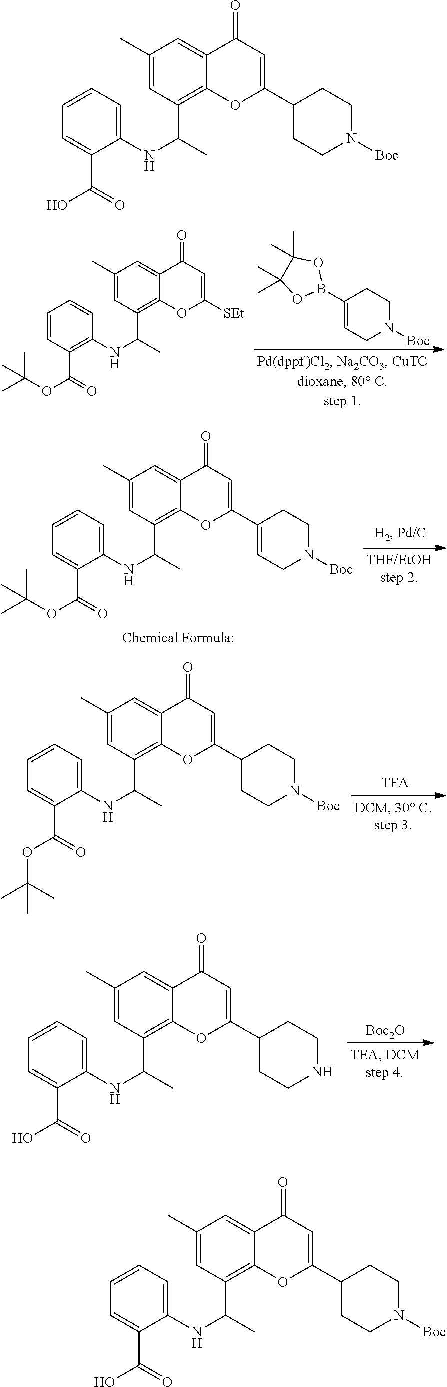

### Step 1

A mixture of tert-butyl 2-[I-(2-ethylsulfanyl-6-methyl-4-oxo-chromen-8-yl)ethylamino]-benzoate (220 mg, 500.49 μmol), tert-butyl 4-(4,4,55-tetramethyl-1,3,2-dioxaborolan-2-yl)-3,6-dihydro-2H-pyridine-1-carboxylate (201.18 mg, 650.64 μmol), Pd(dppf)Cl₂ (146.49 mg, 200.20 μmol), thiophene-2-carbonyloxycopper (190.88 mg, 1.00 mmol) and Na₂CO₃ (106.09 mg, 1.00 mmol) in dioxane (4 mL) was degassed and purged with N₂ for 3 times, and then the mixture was stirred at 80° C. for 15 h under N₂ atmosphere. The reaction mixture was combined with ES20987-64 and concentrated under reduced pressure to give a residue. The residue was purified by flash silica gel chromatography to give tert-butyl 4-[8-[1-(2-tert-butoxycarbonylanilino)ethyl]-6-methyl-4-oxo-chromen-2-yl]-3,6-dihydro-2H-1-pyridine-1-carboxylate (180 mg, 90% purity) as a yellow solid.

MS m/z (ESI): 561 [M+H]⁺.

### Step 2

To a solution of tert-butyl 4-[8-[1-(2-tert-butoxycarbonylanilino)ethyl]-6-methyl-4-oxo-chromen-2-yl]-3,6-dihydro-2-H-pyridine-1-carboxylate (140 mg, 249.70 μmol) in THF (1 mL) was added EtOH (1 mL) and Pd/C (10 mg, 10% purity) under N2 atmosphere. The suspension was degassed and purged with H₂ for 3 times. The mixture was stirred under 1-12 (15 Psi) at 20° C. for 0.5 h. The mixture was combined with ES20987-68 and filtered through Celite and concentrated under reduced pressure to give crude tert-butyl 4-[8-[1-(2-tert-butoxycarbonylanilino)ethyl]-6-methyl-4-oxo-chromen-2-yl]piperidine-1-carboxylate (130 mg) as yellow solid.

MS m/z (ESI): 563 [M+H]⁺.

### Step 3

To a solution of tert-butyl 4-[8-[I-(2-tert-butoxycarbonylanilino)ethyl]-6-methyl-4-oxo-chromen-2-yl]piperidine-1-carboxylate (40 mg, 71.09 μmol) in DCM (0.2 mL) was added TFA (308.00 mg, 2.70 mmol, 200.00 uL). The mixture was stirred at 20° C. for 15 h. The mixture was stirred at 30° C. for 5 h. The reaction mixture was concentrated under reduced pressure to give 2-[1-[6-methyl-4-oxo-2-(4-piperidyl)chromen-8-yl] ethylamino]benzoic acid (28 mg, crude) as yellow gum.

MS m/z (ESI): 407 [M+H]⁺.

### Step 4

To a solution of 2-[1-[6-methyl-4-oxo-2-(4-piperidyl)chromen-8-yl]ethylamino]benzoic acid (28 mg, 68.89 μmol) in DCM (1 mL) was added (Boc)₂O (3007 mg, 137.77 μmol) and TEA (20.91 mg, 206.66 μmol, 28.76 uL). The mixture was stirred at 20° C. for 2 h. The reaction mixture was concentrated under reduced pressure to give a residue. The residue was purified by prep-HPLC (column: Boston Green ODS 150*30 mm*5 um; mobile phase: [water (FA)-ACN]; 13%: 53%-83%, 6 min) to give 2-[1-[2-(1-tert-butoxycarbonyl-4-piperidyl)-6-methyl-4-oxo-chromen-8-yl]ethylamino]benzoic acid (4.4 mg, 8.64 μmol, 12.6% yield, 99.5% purity) as white solid. MS m/z (ESI): 507 [M+H]⁺.

¹H NM/R (400 MHz, DMSO-d₆) δ ppm 8.40 (d, J=2.0 Hz, 1H), 7.81-7.79 (m, 1H), 7.67 (s, 1H), 7.51 (d, J=2.4 Hz, 1H), 7.23-7.19 (m, 1H), 7.09 (s, 1H), 6.55-6.52 (m, 1H), 6.46-6.44 (m, 1H), 6.22 (s, 1H), 5.14-5.11 (m, 1H), 4.06-4.04 (m, 2H), 2.91-2.85 (m, 2H), 2.32 (s, 3H), 2.00-1.94 (m, 2H), 1.59-1.45 (m, 6H), 1.39 (s, 9H).

### Example 14

### 2-((1-(2-(1-(tert-butoxy carbonyl)-1,2,3,6-tetrahydropyridin-4-yl)-6-methyl-4-oxo-4H-chromen-8-yl)ethyl)amino)benzoic acid

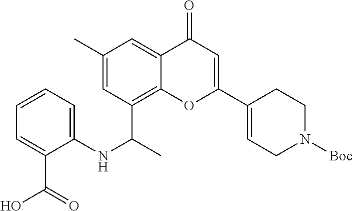 [SMILES: Cc1cc(C(C)Nc2ccccc2C(=O)O)c2oc(C3=CCN(C(=O)OC(C)(C)C)CC3)cc(=O)c2c1]

2-((1-(2-(1-(tert-butoxycarbonyl)-1,2,3,6-tetrahydropyridin-4-yl)-6-methyl-4-oxo-4H-chromen-8-yl)ethyl)amino)benzoic acid was prepared in accordance with the method of Example 13.

MS mm/z (ESI): 505[M+H]⁺.

¹H NMR (400 MHz, CD3OD-d4) δ ppm 7.94-7.92 (m, 1H), 7.81 (s, 1H), 7.61 (d, J=16 Hz, 1H), 7.23-7.19 (m, 1H), 6.99 (br s, 1H), 6.59-6.55 (M, 1H), 6.49-6.47 (m, 1H), 6.42 (s, 1H), 5.28-5.24 (m, 1H), 4.21 (br s, 2H), 3.68 (br s, 2H), 2.53 (br s, 2H), 2.40 (s, 3H), 1.72 (d, J=6.8 Hz, 3H), 1.52 (s, 9H).

### Example 15

### 2-((1-(6-methyl-4-oxo-2-(piperidin-4-yl)-4H-chromen-8-yl)ethyl)amino)benzoic acid

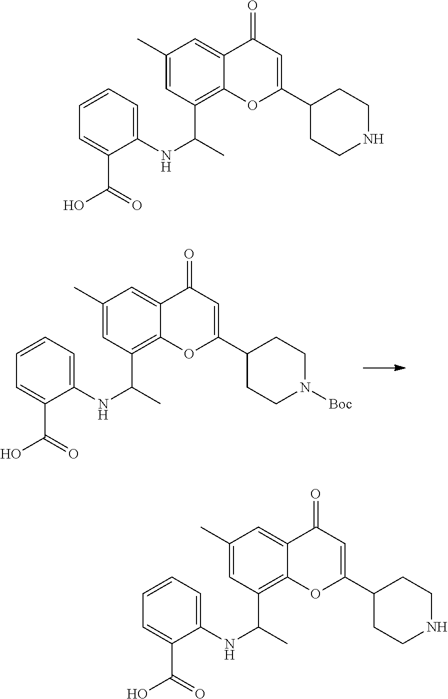 [SMILES: Cc1cc(C(C)Nc2ccccc2C(=O)O)c2oc(C3CCNCC3)cc(=O)c2c1 ; Cc1cc(C(C)Nc2ccccc2C(=O)O)c2oc(C3CCN(C(=O)OC(C)(C)C)CC3)cc(=O)c2c1 ; Cc1cc(C(C)Nc2ccccc2C(=O)O)c2oc(C3CCNCC3)cc(=O)c2c1]

To a solution of 2-((1-(2-(1-(ter-butoxycarbonyl)piperidin-4-yl)-6-methyl-4-oxo-4-1-chromen-8-yl)ethyl)amino)benzoic acid (1.0 mmol) in 10 mL DCM was added TFA (5 mL). The mixture was stirred at rt for 2 h. The mixture was concentrated and purified by prep-HPLC to give the product.

MS: m/z 407.2 [M+H]⁺.

¹H NMR (400 MHz, DMSO-d6) δ ppm 9.34 (br s, 1H), 7.79 (d, J=7.2 Hz, 1H), 7.74 (s, 1H), 7.68 (s, 1H), 7.08-7.04 (m, 1H), 6.47 (d, J=8.0 Hz, 1H), 6.42-6.38 (m, 11H), 6.24 (s, 1H), 4.90 (br s, 1H), 3.53-3.50 (m, 2H), 3.38-3.35 (m, 2H), 3.07-2.91 (m, 2H), 2.40 (s, 3H), 2.09-1.80 (m, 3H), 1.62 (d, J=6.8 Hz, 3H).

### Example 16

### 2-((1-(6-methyl-4-oxo-2-(1-(1,1,1-trifluoropropan-2-yl)piperidin-4-yl)-4H-chromen-8-yl)ethyl)amino)benzoic acid

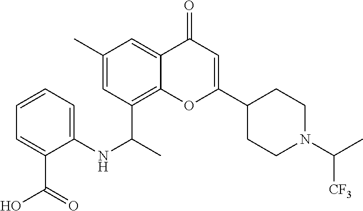 [SMILES: Cc1cc(C(C)Nc2ccccc2C(=O)O)c2oc(C3CCN(C(C)C(F)(F)F)CC3)cc(=O)c2c1]

2-((1-(6-methyl-4-oxo-2-(1-(1,1,1-trifluoropropan-2-yl)piperidin-4-yl)-4H-chromen-8-yl)ethyl)amino)benzoic acid was prepared in accordance with the method of Example 13.

MS: m/z 503.2 [M+H]⁺;

¹H NMR (400 MHz, DMSO-dr) δ ppm 8.41 (d, J=5.2 Hz, 1H), 7.81 (d, J=8.0 Hz, 1H), 7.69 (s, 1H), 7.52 (s, 1H), 7.25-7.21 (m, 1H), 6.57-6.53 (m, 1H), 6.46 (d, J=8.4 Hz, 1H), 6.23 (s, 1H), 5.16-5.13 (m, 1H), 3.52-3.48 (m, 2H), 3.04-2.97 (m, 2H), 2.74-2.64 (m, 2H), 2.34 (s, 3H), 2.00 (br s, 3H), 1.69-1.59 (m, 5H), 1.19 (d, J=7.2 Hz, 3H).

### Example 17

### 2-((1-(6-methyl-4-oxo-2-(1-(1,1-trifluoropropan-2-yl)-1,2,3,6-tetrahydropyridin-4-yl)-4H-chromen-8-yl)ethyl)amino)benzoic acid

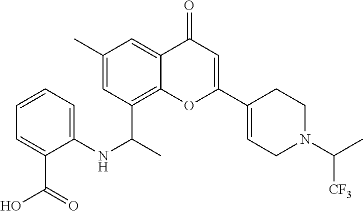 [SMILES: Cc1cc(C(C)Nc2ccccc2C(=O)O)c2oc(C3=CCN(C(C)C(F)(F)F)CC3)cc(=O)c2c1]

2-((l-(6-methyl-4-oxo-2-(1-(1,1,1-trifluoropropan-2-yl)-1,2,3,6-tetrahydropyridin-4-yl)-4H-chromen-8-yl)ethyl)amino)benzoic acid was prepared in accordance with the method of Example 13.

LC-MS: mm/z 501.2 [M+H-]⁺;

¹H NMR (400 MHz, DMSO-d₆) δ ppm 8.42 (br s, 1H), 7.82 (d, J=7.6 Hz, 1H), 7.69 (s, 1H), 7.52 (s, 1H), 7.24-7.21 (m, 1H), 6.98 (br s, 1H), 6.57-6.54 (m, 1H), 6.44 (d, J=8.4 Hz, 1H), 6.36 (s, 1H), 5.23-5.20 (m, 1H), 3.66-3.56 (in 4H), 2.93-2.87 (m, 2H), 245 (br s, 2H), 2.34 (s, 3H) 1.61 (d, J=6.8 Hz, 3H), 1.23 (d, J=7.2 Hz, 3H).

### Example 18

### 2-((1-(6-methyl-4-oxo-2-(1-phenylpiperidin-4-yl)-4H-chromen-8-yl)ethyl)amino)benzoic acid

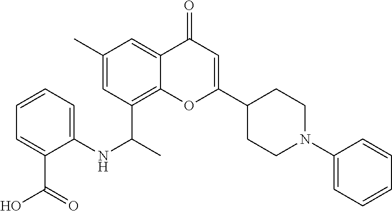 [SMILES: Cc1cc(C(C)Nc2ccccc2C(=O)O)c2oc(C3CCN(c4ccccc4)CC3)cc(=O)c2c1]

2-((1-(6-methyl-4-oxo-2-(1-phenylpiperidin-4-yl)-4H-chromen-8-yl)ethyl)amino)benzoic acid was prepared in accordance with the method of Example 13.

MS: m/z 483.3 [M+H]⁺;

¹H NMR (400 MHz, DMSO-d₆) δ ppm 8.54 (br s, 1H), 7.81 (d, J=7.6 Hz, 1H), 7.70 (s, 1H), 7.54 (s, 1H), 7.23-7.20 (m, 3H), 7.00-6.97 (m, 2H), 6.79-6.76 (m, 1H), 6.55-6.44 (m, 2H), 6.26 (s, 1H), 5.13 (br s, 1H), 3.83-3.80 (m, 2H), 2.89-2.79 (m, 2H), 2.34 (s, 3H), 2.10-2.07 (m, 2H), 1.87-1.84 (m, 2H), 1.59 (d, J=6.4 Hz, 3H).

### Example 19

### 2-((1-(6-methyl-4-oxo-2-(1-phenyl-1,2,3,6-tetrahydropyridin-4-yl)-4H-chromen-8-yl)ethyl)amino)benzoic acid

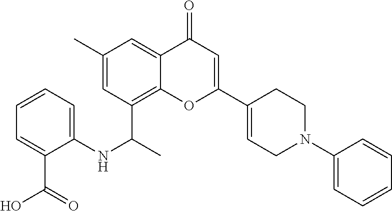 [SMILES: Cc1cc(C(C)Nc2ccccc2C(=O)O)c2oc(C3=CCN(c4ccccc4)CC3)cc(=O)c2c1]

2-((1-(6-methyl-4-oxo-2-(1-phenyl-1,2,3,6-tetrahydropyridin-4-yl)-4H-chromen-8-yl)ethyl)amino)benzoic acid was prepared in accordance with the method of Example 13.

MS: m/z 481.3 [M+H]⁺;

¹H NMR (400 MHz, DMSO-d₆) δ ppm 8.45 (br s, 1H), 7.82 (d, J=6.4 Hz, 1H), 7.70 (s, 1H), 7.54 (s, 1H), 7.26-7.23 (m, 3H), 7.11 (br s, 1H), 7.02-6.99 (m, 2H), 6.79-6.76 (m, 1H), 6.55-6.46 (m, 2H), 6.43 (s, 1H), 5.24-5.21 (m, 1H), 3.98 (br s, 2H), 3.50-3.47 (m, 2H), 2.67-2.66 (m, 1H), 2.34-2.32 (m, 4H), 1.64 (d, J=6.8 Hz, 3H).

### Example 20

### 2-((1-(3,6-dimethyl-2-(2-methyl-1-oxoisoindolin-5-yl)-4-oxo-4H-chromen-8-yl)ethyl)amino)benzoic acid

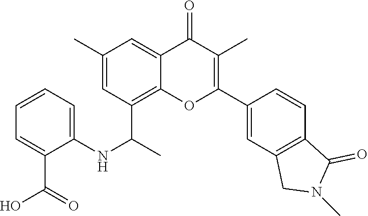 [SMILES: Cc1cc(C(C)Nc2ccccc2C(=O)O)c2oc(-c3ccc4c(c3)CN(C)C4=O)c(C)c(=O)c2c1]

2-((1-(3,6-dimethyl-2-(2-methyl-1-oxoisoindolin-5-yl)-4-oxo-4H-chromen-8-yl)ethyl)amino)benzoic acid was prepared in accordance with the method of Example 1.

MS: m/z 483.3 [M+H]⁺;

¹H NMR (400 MHz, DMSO-d₆) δ ppm 8.57 (br s, 1H), 8.00 (s, 1H), 7.90-7.79 (m, 4H), 7.56 (s, 1H), 7.18-7.15 (m, 1H), 6.52-6.44 (m, 2H), 5.10 (br s, 1H), 4.58 (s, 2H), 3.11 (s, 3H), 2.37 (s, 3H), 2.08 (s, 3H), 1.57 (d, J=6.0 Hz, 3H).

The stereoisomers of example 20: (S)-2-((1-(3,6-dimethyl-2-(2-methyl-1-oxoisoindolin-5-yl)-4-oxo-4H-chromen-8-yl)ethyl)amino)benzoic acid and (R)-2-((1-(3,6-dimethyl-2-(2-methyl-1-oxoisoindolin-5-yl)-4-oxo-4H-chromen-8-yl)ethyl)amino)benzoic acid

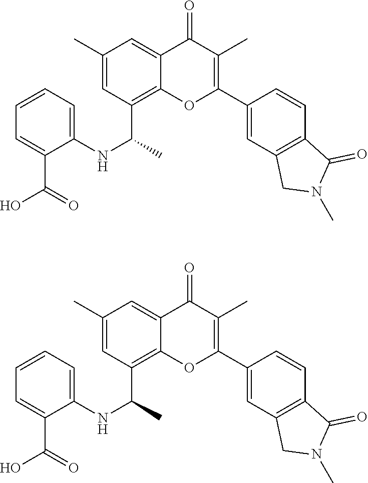 [SMILES: Cc1cc([C@H](C)Nc2ccccc2C(=O)O)c2oc(-c3ccc4c(c3)CN(C)C4=O)c(C)c(=O)c2c1 ; Cc1cc([C@@H](C)Nc2ccccc2C(=O)O)c2oc(-c3ccc4c(c3)CN(C)C4=O)c(C)c(=O)c2c1]

The stereoisomers of example 20 were prepared with chiral SFC (column: DAICEL CHIRALCEL OD (250 mm*30 mm, 10 um) and arbitrarily assigned

MS: m/z 483.2 [M+H]⁺; ¹H NMR (400 MHz, DMSO-d₆) δ ppm 12.74 (br s, 1H), 8.36 (br s, 1H), 7.99 (s, 1H), 7.87-7.79 (m, 4H), 7.56 (s, 1H), 7.22-7.19 (m, 1H), 6.56-6.46 (m, 2H), 5.12-5.09 (m, 1H), 4.58 (s, 2H), 3.12 (s, 3H), 2.38 (s, 3H), 2.08 (s, 3H), 1.58 (d, J=6.8 Hz, 3H). SFC (column: DAICEL CHIRALCEL OD (250 mm*30 mm, 10 um); mobile phase: [Neu-EtOH]; B %: 45%-45%, min). Rt:==1.59 min.

MS: mm/z 483.2 [M+H]⁺; ¹H NMR (400 MHz, DMSO-d₆) δ ppm 12.74 (br s, 1H), 8.37 (br s, 1H), 7.99 (s, 1H), 7.89-7.79 (m, 4H), 7.56 (s, 1H), 7.22-7.19 (n, 1H), 6.56-6.46 (m, 2H), 5.12-5.09 (m, 1H), 4.58 (s, 2H), 3.12 (s, 3H), 2.38 (s, 3H), 2.08 (s, 3H), 1.58 (d, J=6.8 Hz, 3H). SFC (column: DAICEL CHIRALCEL OD (250 mm*30 mm, 10 um); mobile phase: [Neu-EtOH]; B %: 45%-45%, min) Rt=3.25 min

Examples 21-53 were prepared in a similar manner as the preparation of example 95, and their stereoisomers can be prepared with chiral SFC in a similar manner as the preparation of the stereoisomers of example 20.

### Example 21

### 2-((1-(6-methyl-2-(1-methyl-2-oxoindolin-6-yl)-4-oxo-4H-chromen-8-yl)ethyl)amino)benzoic acid

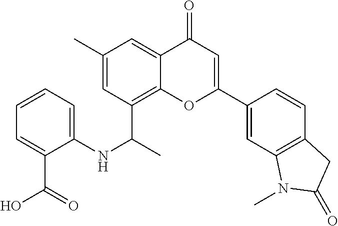 [SMILES: Cc1cc(C(C)Nc2ccccc2C(=O)O)c2oc(-c3ccc4c(c3)N(C)C(=O)C4)cc(=O)c2c1]

MS mm/z (ESI): 469 [M+H]⁺.

¹H NM (400 MHz, DMSO-d₆) δ ppm 12.78 (br s, 1H), 8.44 (d, J=6.4 Hz, 1H), 7.82-7.76 (m, 3H), 7.68 (s, 1H), 7.58 (s, 1H), 7.45 (d, J=8.0 Hz, 1H), 7.24-7.19 (m, 2H), 6.58-6.54 (m, 2H), 5.36-5.33 (m, 1H), 3.37 (s, 2H), 3.21 (s, 3H), 2.38 (s, 3H), 1.70 (d, J=6.8 Hz, 3H).

### Example 22

### 6-chloro-3-((1-(3,6-dimethyl-2-(2-methyl-1-oxoisoindolin-5-yl)-4-oxo-4H-chromen-8-yl) ethyl)amino)picolinic acid

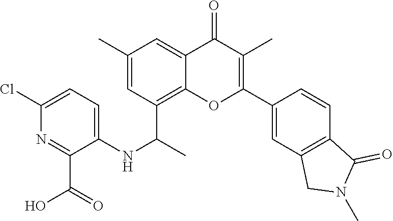 [SMILES: Cc1cc(C(C)Nc2ccc(Cl)nc2C(=O)O)c2oc(-c3ccc4c(c3)CN(C)C4=O)c(C)c(=O)c2c1]

MS: m/z 518.2 [M+H]⁺;

¹H NMR (400 MHz, DMSO-d₆) δ ppm 8.29-8.25 (m, 1H), 7.97 (s, 1H), 7.86-7.80 (m, 1H), 7.75 (s, 1H), 7.56 (d, J=2.4 Hz, 1H), 7.30 (d, J=8.8 Hz, 1H), 7.09 (d, J=8.8 Hz, 1H), 5.16-5.12 (m, 1H), 4.57 (s, 2H), 3.12 (s, 3H), 2.38 (s, 3H), 2.07 (s, 3H), 1.60 (d, J=6.81 Hz, 3H).

### Example 23

### 2-((1-(6-methyl-2-(2-methyl-3-oxoisoindolin-5-yl)-4-oxo-4H-chromen-8-yl)ethyl)amino)benzoic acid

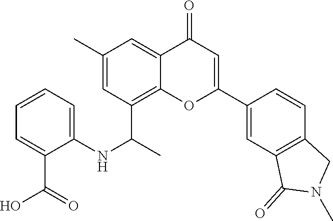 [SMILES: Cc1cc(C(C)Nc2ccccc2C(=O)O)c2oc(-c3ccc4c(c3)C(=O)N(C)C4)cc(=O)c2c1]

MS: m/z 469.3 [M+H]⁺;

¹H NMR (400 MHz, DMSO-d₆) δ ppm 12.80 (br s, 1H), 8.45 (d, J=5.6 Hz, 1H), 8.34 (d, J=5.6 Hz, 1H), 7.83-7.76 (m, 3H), 7.55 (s, 1H), 7.61-7.56 (m, 2H), 7.23-7.19 (m, 1H), 6.58-6.51 (n, 2H), 5.36-5.33 (n, 1H), 4.58 (s, 2H), 311 (s, 3H), 2.36 (s, 13H), 1.68 (d, J=6.8 Hz, 3H).

### Example 24

### 2-((1-(6-methyl-2-(2-methyl-1-oxoisoindolin-4-yl)-4-oxo-4H-chromen-8-yl)ethyl)amino)benzoic acid

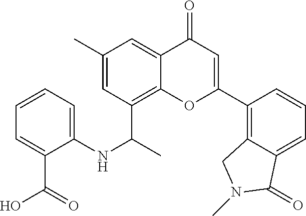 [SMILES: Cc1cc(C(C)Nc2ccccc2C(=O)O)c2oc(-c3cccc4c3CN(C)C4=O)cc(=O)c2c1]

MS: m/z 469.3 [M+H]⁺;

¹H NMR (400 MHz, DMSO-d₆) δ ppm 8.48 (br s, H), 8.23 (d, J=7.6 Hz, 1H), 7.90 (d, J=7.6 Hz, 1H), 7.83-7.78 (m, 3H), 7.59 (d, J=1.6 Hz, 1H), 7.22-7.18 (m, 1H), 6.89 (s, 1H), 6.57-6.46 (m, 2H), 5.32 (br s, 1H), 5.01-4.90 (m, 2H), 3.08 (s, 2H), 2.38 (s, 3H), 1.66 (d, J=6.4 Hz, 3H).

### Example 25

### 2-((1-(2-(1H-indol-2-yl)-6-methyl-4-oxo-411-chromen-8-yl)ethyl)amino)benzoic acid

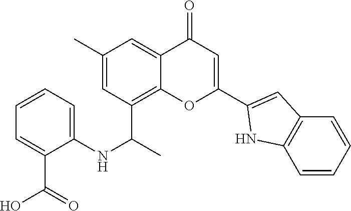 [SMILES: Cc1cc(C(C)Nc2ccccc2C(=O)O)c2oc(-c3cc4ccccc4[nH]3)cc(=O)c2c1]

MS: m/z 439.3 [M+H]⁺;

¹H NMR (400 MHz, DMSO-d₆) δ ppm 12.82 (br s, 1H), 11.94 (s, 1H), 8.47 (d, J=6.0 Hz, 1H), 7.82-7.67 (m, 3H), 7.55-7.51 (m, 2H), 7.42 (s, 1H), 7.28-7.11 (m, 2H), 7.02 (s, 1H), 6.57-6.54 (m, 2H), 5.50-5.46 (m, 1H), 2.36 (s, 3H), 1.68 (d, J=6.8 Hz, 3H).

### Example 26

### 2-((1-(6-methyl-4-oxo-2-(thiophen-2-yl)-4H-chromen-8-yl)ethyl)amino)benzoic acid

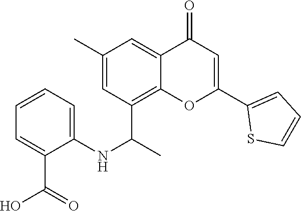 [SMILES: Cc1cc(C(C)Nc2ccccc2C(=O)O)c2oc(-c3cccs3)cc(=O)c2c1]

MS: m/z 406.1 [M+H]⁺;

¹H NMR (400 MHz, DMSO-d₆) δ ppm 12.81 (br s, 1H), 8.45 (d, J=6.0 Hz, 1H), 8.09 (d, J=: 4.0 Hz, 1H), 8.00 (d, J=4.8 Hz, 1H), 7.83-7.81 (m, 1H), 7.72 (s, 1H), 7.54 (s, 1H), 7.33-7.18 (m, 2H), 6.97 (s, 1H), 6.56-6.49 (m, 2H), 5.26-5.22 (m, 1H), 2.36 (s, 3H), 1.69 (d, J=6.4 Hz, 3H).

### Example 27

### 2-((1-(6-methyl-4-oxo-2-(thiazol-5-yl)-4H-chromen-8-yl)ethyl)amino)benzoic acid

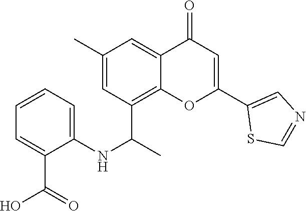 [SMILES: Cc1cc(C(C)Nc2ccccc2C(=O)O)c2oc(-c3cncs3)cc(=O)c2c1]

MS: m/z 407.2 [M+H]⁺;

¹H NMR (400 MHz, DMSO-d₆) δ ppm 9.41 (s, 1H), 8.34 (s, 1H), 8.44 (d, J=6.4 Hz, 1H), 7.82-7.80 (m, 1H), 7.73 (s, 1H), 7.55 (d, J=2.0 Hz, 1H), 7.25-7.21 (m, 1H), 7.07 (s, 1H), 6.57-6.51 (M, 2H), 5.24-5.21 (i, 1H), 2.35 (s, 3H), 1.67 (d, J=6.8 Hz, 3H).

### Example 28

### 2-((1-(2-(1-acetylpiperidin-4-yl)-6-methyl-4-oxo-4H-chromen-8-yl)ethyl)amino)benzoic acid

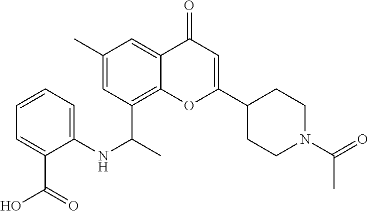 [SMILES: CC(=O)N1CCC(c2cc(=O)c3cc(C)cc(C(C)Nc4ccccc4C(=O)O)c3o2)CC1]

MS: m/z 449.2 [M+H]⁺;

¹H NMR (400 MHz, DMSO-d₆) δ ppm 8.44 (br s, 1H), 7.82 (d, J=7.6 Hz, 1H), 7.70 (s, 1H), 7.54 (s, 1H), 7.25-7.21 (m, 1H), 6.57-6.53 (m, 1H), 6.47 (d, J=8.8 Hz, 1H), 6.23 (s, 1H), 5.15 (br s, 1H), 4.53-4.49 (m, 1H), 3.94 (br s, 1H), 3.18-315 (m, 1H), 3.00-294 (m, 1H), 2.68-2.61 (m, 1H), 2.35 (s, 3H), 2.04-2.00 (m, 5H), 1.68-1.50 (m, 5H).

### Example 29

### 2-((1-(6-methyl-4-oxo-2-(1-(2,2,2-trifluoroacetyl)piperidin-4-yl)-4H-chromen-8-yl)ethyl)amino)benzoic acid

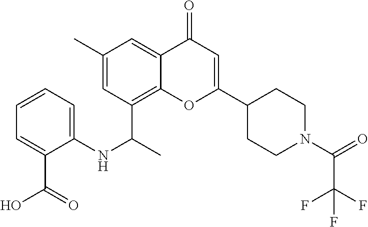 [SMILES: Cc1cc(C(C)Nc2ccccc2C(=O)O)c2oc(C3CCN(C(=O)C(F)(F)F)CC3)cc(=O)c2c1]

MS: mm/z 503.0 [M+H]⁺;

¹H NMR (400 MHz, DMSO-d₆) δ ppm 8.42 (br s, 1H), 7.82-7.80 (m, 1H), 7.69 (s, 1H), 7.57 (s, 1H), 7.24-7.21 (n, 1H), 6.57-6.53 (m, 1H), 6.47 (d, J=8.8 Hz, 1H), 6.23 (s, 1H), 5.18-5.15 (i, 1H), 4.43-4.41 (m, 1H), 4.00-3.96 (m, 1H), 3.94 (br s, 1H), 3.13-3.00 (m, 2H), 2.35 (s, 3H), 2.17-2.10 (M, 2H), 1.75-1.69 (M, 2H), 1.60 (d, J=6.4 Hz, 3H).

### Example 30

### 2-((1-(2-(1-(cyclopropanecarbonyl)piperidin-4-yl)-6-methyl-4-oxo-4H-chromen-8-yl)ethyl)amino)benzoic acid

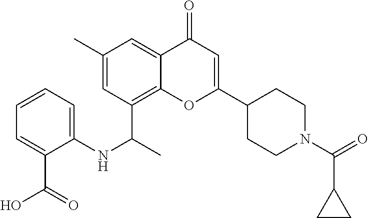 [SMILES: Cc1cc(C(C)Nc2ccccc2C(=O)O)c2oc(C3CCN(C(=O)C4CC4)CC3)cc(=O)c2c1]

MS: m/z 4175.0 [M+H]⁺;

¹H NMR (400 MHz, DMSO-d₆) δ ppm 8.53 (br s, 1H), 7.82-7.80 (m, 1H), 7.69 (s, 1H), 7.54 (d, J=: 2.0 Hz, 1H), 7.23-7.19 (m, 1H), 6.55-6.52 (m, 1H), 6.45 (d, J=7.6 Hz, 1H), 6.25 (s, 1H), 5.15-5.13 (m, 1H), 4.49-4.41 (m, 2H), 3.22-3.18 (m, 2H), 3.04-2.99 (m, 1H), 2.68-2.61 (n, 1H), 2.34 (s, 3H), 2.08-2.02 (n, 4H), 1.68-1.50 (m, 5H), 0.72-0.69 (m, 4H).

### Example 31

### 2-((1-(6-methyl-4-oxo-2-(pyridin-2-yl)-4H-chromen-8-yl)ethyl)amino)benzoic acid

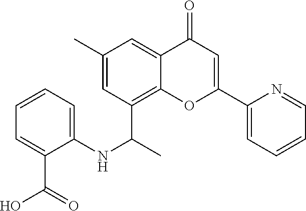 [SMILES: Cc1cc(C(C)Nc2ccccc2C(=O)O)c2oc(-c3ccccn3)cc(=O)c2c1]

¹H NMR (400 MHz, DMSO-d₆) δ ppm 12.54-13.09 (m, 1H) 8.81 (br d, J=4.25 Hz, 1H) 8.46 (br d, J=6.00 Hz, 1H) 8.27 (d, J=7.88 Hz, 1H) 8.07 (br t, J=7.19 Hz, 1H) 7.74-7.86 (m, 2H) 7.64 (br dd, J=7.00, 4.88 Hz, 1H) 7.58 (s, 1H) 7.18-7.28 (m, 2H) 6.55 (br d, J=8.00 Hz, 2H) 5.39 (br t, J=6.44 Hz, 1H) 2.38 (s, 3H) 1.70 (br d, J=6.63 Hz, 3H)

MS m/z (ESI): 401 [M+H]⁺.

### Example 32

### 2-((1-(6-methyl-4-oxo-2-(pyridin-3-yl)-4H-chromen-8-yl)ethyl)amino)benzoic acid

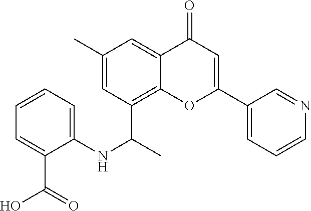 [SMILES: Cc1cc(C(C)Nc2ccccc2C(=O)O)c2oc(-c3cccnc3)cc(=O)c2c1]

¹H NMR (400 MHz, DMSO-d₆) δ ppm 9.33 (s, 1H), 8.79 (d, J=4.02 Hz, 1H), 8.50-8.69 (m, 2H), 7.75-7.87 (m, 2H), 7.55-7.68 (m, 2H), 7.17-7.27 (m, 2H), 6.47-6.59 (m, 2H), 5.35 (br s, 1H), 2.37 (s, 3H), 1.68 (d, J=6.78 Hz, 3H) MS m/z (ESI): 401 [M-+H]⁺.

### Example 33

### 2-((1-(6-methyl-4-oxo-2-(pyridin-4-yl)-4H-chromen-8-yl)ethyl)amino)benzoic acid

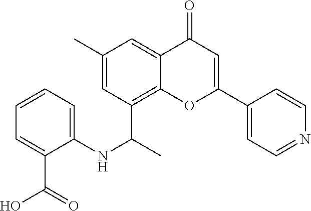 [SMILES: Cc1cc(C(C)Nc2ccccc2C(=O)O)c2oc(-c3ccncc3)cc(=O)c2c1]

¹H NMR (400 MHz, DMISO-d₆) δ ppm 8.53-8.89 (m, 3H), 8.09 (d, J=6.02 Hz, 2H), 7.75-7.85 (m, 2H), 7.60 (s, 1H), 7.31 (s, 1H), 7.18 (br t, J=7.28 Hz, 1H), 6.46-6.58 (m, 2H), 5.35 (br s, 1H), 2.37 (s, 3H), 1.68 (d, J=6.53 Hz, 3H)

MS m/z (ESI): 401 [M+H]⁺.

### Example 34

### 2-((1-(6-methyl-2-(1-methyl-2-oxo-1,2-dihydropyridin-4-yl)-4-oxo-4H-chromen-8-yl)ethyl)amino)benzoic acid

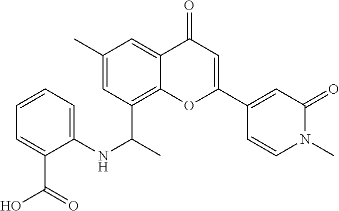 [SMILES: Cc1cc(C(C)Nc2ccccc2C(=O)O)c2oc(-c3ccn(C)c(=O)c3)cc(=O)c2c1]

¹H NMR (400 MHz, DMSO-d) δ ppm 12.46-13.03 (n, 1H) 8.42-8.54 (m, 1H) 7.89 (d, J=7.13 Hz, 1H) 7.82 (br d, J=7.75 Hz, 1H) 7.74 (s, 1H) 7.58 (brs, 1H) 7.55 (d, J=1.50 Hz, 1H) 7.21 (br t, J=7.32 Hz, 1H) 7.16 (s, 1H) 7.13 (d, J=1.38 Hz, 1H) 6.87 (dd, J=7.07, 1.69 Hz, 1H) 6.56 (t, J=7.50 Hz, 1H) 6.48 (br d, J=8.38 Hz, 1H) 5.26-5.34 (m, 1H) 3.50 (s, 3H) 2.34-2.37 (m, 3H) 1.65 (br d, J=6.50 Hz, 3H) MS m/z (ESI): 430[M+H]⁺.

### Example 35

### 2-((1-(6-methyl-4-oxo-2-(quinolin-6-yl)-4H-chromen-8-yl)ethyl)amino)benzoic acid

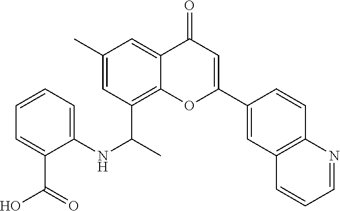 [SMILES: Cc1cc(C(C)Nc2ccccc2C(=O)O)c2oc(-c3ccc4ncccc4c3)cc(=O)c2c1]

¹H NMR (400 MHz, DMSO-d₆) δ ppm 12.77 (br s, 1H) 9.02 (dd, J=4.13, 1.50 Hz, 1H) 8.82 (d, J=1.50 Hz, 1H) 8.58 (br d, J=8.00 Hz, 1H) 8.39-8.53 (m, 2H) 8.17 (d, J=8.88 Hz, 1H) 7.75-7.86 (m, 2H) 7.57-7.70 (m, 2H) 7.19-7.31 (m, 2H) 6.52-6.65 (m, 2H) 5.46 (br t, J=6.25 Hz, 1H) 2.39 (s, 3H) 1.73 (d, J=6.63 Hz, 3H)

MS m/z (ESI): 451 [M+H]⁺.

### Example 36

### 2-((1-(2-(isoquinolin-6-yl)-6-methyl-4-oxo-4H-chromen-8-yl)ethyl)amino)benzoic acid

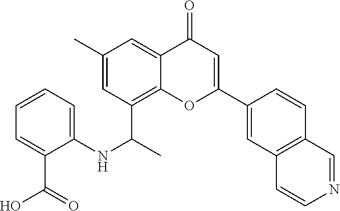 [SMILES: Cc1cc(C(C)Nc2ccccc2C(=O)O)c2oc(-c3ccc4cnccc4c3)cc(=O)c2c1]

¹H NMR (400 MHz, DMSO-d₆) δ ppm 12.78 (br s, 1H) 9.45 (br s, 1H) 8.78 (s, 1H) 8.58-8.68 (m, 1H) 8.46 (br d, J=6.25 Hz, 1H) 8.29-8.39 (m, 2H) 8.03 (br d, J=5.75 Hz, 1H) 7.83 (dd, J=7.94, 1.56 Hz, 1H) 7.79 (d, J=1.38 Hz, 1H) 7.61 (d, J=1.88 Hz, 1H) 7.32 (s, 1H) 7.21-7.27 (m, 1H) 6.52-6.64 (m, 2H) 5.46 (br t, J=6.44 Hz, 1H) 2.39 (s, 3H) 1.72 (d, J=6.50 Hz, 3H)

MS m/z (ESI): 451 [M+H]⁺.

### Example 37

### 2-((1-(6-methyl-2-(1-methyl-1H-indazol-5-yl)-4-oxo-4H-chromen-8-yl)ethyl)amino)benzoic acid

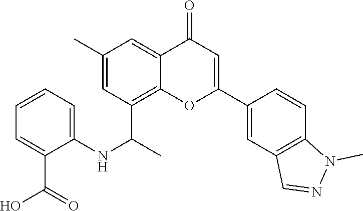 [SMILES: Cc1cc(C(C)Nc2ccccc2C(=O)O)c2oc(-c3ccc4c(cnn4C)c3)cc(=O)c2c1]

¹H NMR (400 MHz, DMSO-d₆) δ ppm 12.78 (br s, 1H), 8.61 (s, 1H), 8.46 (br d, J=6.08 Hz, 1H), 8.24 (s, 1H), 8.14 (dd, J=8.94, 1.55 Hz, 1H), 7.83 (d, J=9.30 Hz, 2H), 7.76 (s, 1H), 7.58 (d, J=2.03 Hz, 1H), 7.22-7.29 (m, 1H), 7.12 (s, 1H), 6.53-6.62 (m, 2H), 5.41 (br t, J=6.32 Hz, 1H), 4.11 (s, 3H), 2.38 (s, 3H), 1.71 (d, J=6.68 Hz, 3H) MS m/z (ESI): 454 [M+H]⁺.

### Example 38

### 2-((1-(6-methyl-2-(2-methyl-2H-imidazol-5-yl)-4-oxo-411-chromen-8-yl)ethyl)amino)benzoic acid

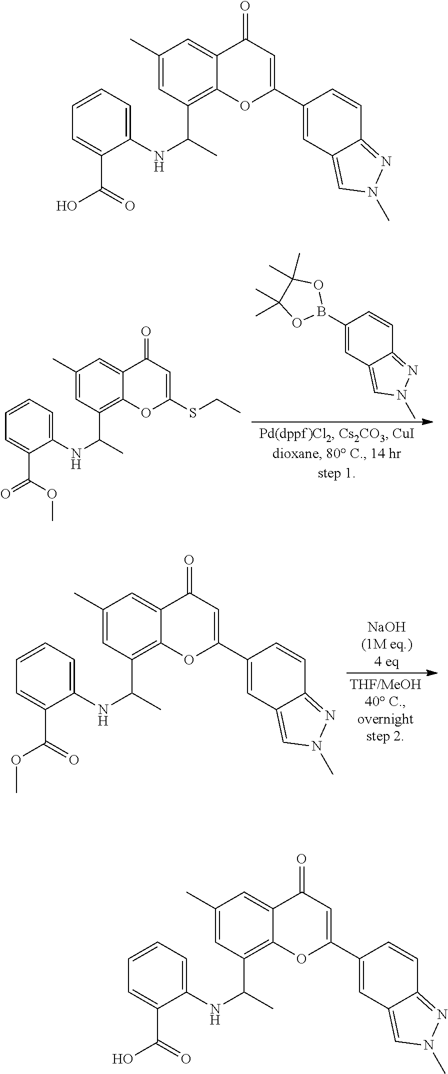

### Step 1

To a solution of methyl 2-[1-(2-ethylsulfanyl-6-methyl-4-oxo-chromen-8-yl) ethylamino]benzoate (50 mg, 125.79 μmol, 1 eq) in dioxane (1 mL) was added 2-methyl-5-(4,4,5,5-tetramethyl-1,3,2-dioxaborolan-2-yl)indazole (48.7 ng, 188.69 μmol, 1.5 eq), Cs2CO3 (82 mg, 251.58 μmol, 2 eq), Pd(dppf)Cl2·CH2Cl2 (20.6 mg, 25.16 μmol, 0.2 eq) and CuI (47.9 mg, 251.58 μmol, 2 eq). The mixture was stirred at 80° C. for 14 hr under N2 atmosphere. LCMS showed 14% desired mass was detected. The mixture was filtered and the filtrate was concentrated tinder vacuum to give methyl 2-[1-[6-methyl-2-(2-methylindazole-5-yl)-4-oxo-chromen-8-yl]ethylamino]benzoate (0.05 g, 14.94 μmol, 12% yield, 14% purity) as a black solid.

MS m/z (ESI: 468 [M+H]⁺.

### Step 2

To a solution of methyl 2-[1-[6-methyl-2-(2-methylindazole-5-yl)-4-oxo-chromen-8-yl]ethylamino]benzoate (50 ng, 14.94 μmol, 1 eq) in THF (0.6 mL) MeOH (0.2 mL) and water (0.2 mL) was added NaOH (1 M, 59.77 uL, 4 eq). The mixture was stirred at 40° C. for 12 hr. LC-MS (ES20586-177-P1B1) showed the completion of the reaction. The reaction mixture was concentrated under reduced pressure to remove solvent. The residue was diluted with water (2 mL) and extracted with EA (2 mL*3). The combined aqueous layers were acidified with 1N HCl to pH=3 and concentrated under reduced pressure to give a residue. The residue was purified by prep-HPLC (column: Welch Xtimate C18 150*30 mm*5 um; mobile phase: [water (FA)-ACN]; B %: 48%-78%, 7 min) to give 2-[1-[6-methyl-2-(2-methylindazole-5-yl)-4-oxo-chromen-8-yl]ethylamino]benzoic acid (1.9 mg, 4.19 gmol, 28% yield, 100% purity) as a white solid.

¹H NMR (400 MHz, DMSO-d₆) δ ppm 12.78 (br s, 1H), 8.59 (d, J=19.83 Hz, 2H), 8.46 (br d, J=6.27 Hz, 1H), 7.92 (br d, J=9.29 Hz, 1H), 7.83 (br d, J=7.03 Hz, 1H), 7.71-7.78 (m, 2H), 7.58 (s, 1H), 7.26 (br t, J=7.28 Hz, 1H), 7.06 (s, 1H), 6.55-6.61 (m, 2H), 5.39 (br t, J=6.53 Hz, 1H), 4.23 (s, 3H), 2.38 (s, 3H), 1.71 (br d, J=6.53 Hz, 3H)

MS m/z (ESI): 454 [M+H]⁺.

The stereoisomers of example 38: (S)-2-((1-(6-methyl-2-(2-menthyl-2H-indazol-5-yl)-4-oxo-411-chromen-8-yl)ethyl)amino)benzoic acid and (R)-2-((1-(6-methyl-2-(2-methyl-211-indazol-5-yl)-4-oxo-4H-chromen-8-yl)ethyl)amino)benzoic acid.

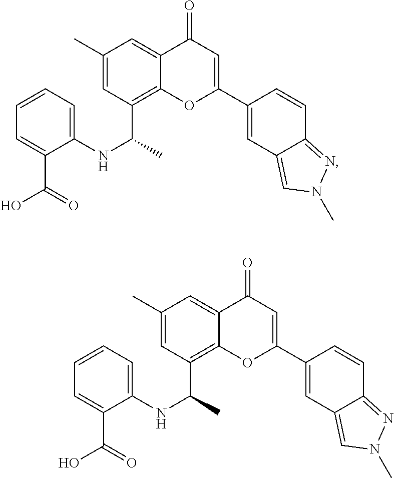 [SMILES: Cc1cc([C@H](C)Nc2ccccc2C(=O)O)c2oc(-c3ccc4nn(C)cc4c3)cc(=O)c2c1 ; Cc1cc([C@@H](C)Nc2ccccc2C(=O)O)c2oc(-c3ccc4nn(C)cc4c3)cc(=O)c2c1]

The stereoisomers of example 38 were prepared in accordance with the method of Example 20 by chiral SFC (column: ChiralpakIB N-3, 250×30 mm I.D., 5 pm; mobile phase: A for CO₂ and B for ETOH (0.1% NH₃·H₂O); B %: 50%-50%, min; Flow rate: 80 mL/min; Back pressure: 100 bar; Column temperature: 40° C.′; Wavelength: 220 nm) to give Peak 1 (Rt=3.06 min) and Peak 2 (Rt=4.03 min).

Peak 1: MS m/z (ESI): 454 [M+H]⁺.

¹H NMR (400 MHz, DMSO-d₆) δ ppm 8.92 (br s, 1H) 8.58 (br d, J=17.26 Hz, 2H) 7.91 (br d, J=9.51 Hz, 1H) 7.85 (br d, J=7.50 Hz, 1H) 7.69-7.77 (m, 2H) 7.56 (s, 1H) 7.15 (br t, J=7.19 Hz, 1H) 7.05 (s, 1H) 6.44-6.60 (m, 2H) 5.36 (br s, 1H) 4.22 (s, 3H) 2.36 (s, 3H) 1.68 (br d, J=6.38 Hz, 3H)

Peak 2: MS m/z (ES1): 454 [M+H]⁺.

¹H NMR (400 MHz, DMSO-'s δ ppm 8.90 (br s, 1H) 8.61 (s, 1H) 8.57 (s, 1H) 7.82-7.94 (m, 2H) 7.68-7.80 (m, 2H) 7.57 (s, 1H) 7.16 (br t, J=7.32 Hz, 1H) 7.05 (s, 1H) 6.44-6.58 (m, 2H) 5.36 (br s, 1H) 4.22 (s, 3H) 2.36 (s, 3H) 1.68 (d, J=6.63 Hz, 3H).

A single crystal of compound 38B was grown in a mixed solution of dichloromethane and methanol (1:1), and its absolute configuration was confirmed by testing as shown in 3813-1:

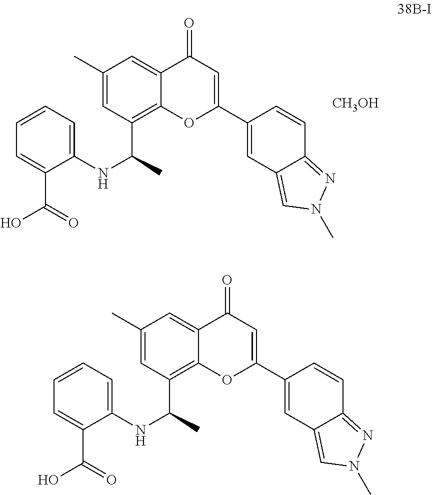

According to the configuration of 38B-1, the absolute configuration of 38B can be confirmed, as shown in the FIGURE:

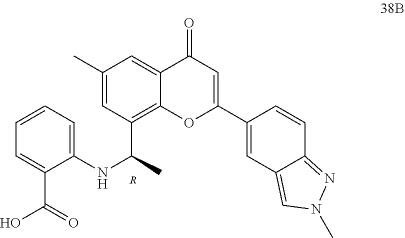

### Example 39

### 2-((1-(2-(isoxazol-4-yl)-6-methyl-4-oxo-4H-chromen-8-yl)ethyl)amino)benzoic acid

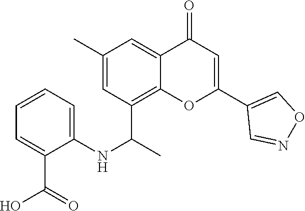 [SMILES: Cc1cc(C(C)Nc2ccccc2C(=O)O)c2oc(-c3cnoc3)cc(=O)c2c1]

¹H N-MR (400 MHz, ACETONITRILE-d₃) δ ppm 1.58 (d, J=6.63 Hz, 3H) 2.28-2.32 (m, 3H) 5.11-5.19 (m, 1H) 6.40-6.47 (m, 1H) 6.51-6.59 (ml, 1H) 7.17-7.28 (m, 2H) 7.39 (br s, 1H) 7.61 (s, 1H) 7.77-7.83 (m, 1H) 8.30-8.52 (m, 1H) 9.13-9.36 (m, 1H) 12.64-12.93 (m, 1H)

MS m/z (ESI): 391 [M+H]⁺.

### Example 40

### 2-((1-(6-methyl-2-(1-(oxetan-3-yl)-1H-pyrazol-4-yl)-4-oxo-4H-chromen-8-yl)ethyl)amino)benzoic acid

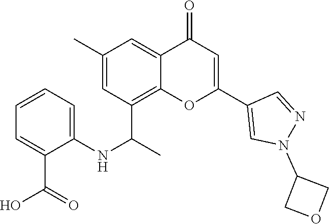 [SMILES: Cc1cc(C(C)Nc2ccccc2C(=O)O)c2oc(-c3cnn(C4COC4)c3)cc(=O)c2c1]

¹H NMR (400 MHz, ACETONITRILE-d₃) δ ppm 1.63 (d, J=6.63 Hz, 3H) 2.34 (s, 3H) 4.89-5.00 (M, 4H) 5.34 (quin, J=6.38 Hz, 1H) 5.68 (quin, J=6.94 Hz, 1H) 6.47-6.60 (m, 2H) 6.80 (s, 1H) 7.18-7.27 (m, 1H) 7.51 (d, J=1.88 Hz, 1H) 7.70 (d, J=1.25 Hz, 1H) 7.81 (dd, J=7.94, 1.44 Hz, 1H) 8.38 (s, 1H) 8.42 (br d, J=6.25 Hz, 1H) 8.74 (s, 1H) 12.57-12.96 (m, 1H) MS m/z (ESI): 446 [M+H]⁺.

### Example 41

### 2-((1-(6-methyl-4-oxo-2-(4-(piperazin-1-yl)phenyl)-4H-chromen-8-yl)ethyl)amino)benzoic acid

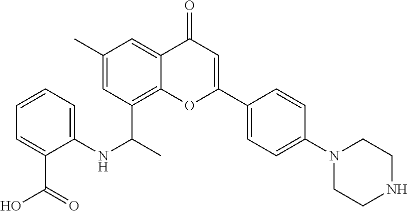 [SMILES: Cc1cc(C(C)Nc2ccccc2C(=O)O)c2oc(-c3ccc(N4CCNCC4)cc3)cc(=O)c2c1]

MS m/z (ESI): 484 [M+H]⁺.

¹H NMR (400 MHz, DMSO-d₆) δ ppm 8.81 (br s, 1H) 8.20 (s, 1H) 7.94-8.03 (m, 2H) 7.82 (d, J=−6.38 Hz, 1H) 7.71 (s, 1H) 7.52 (d J=1.88 Hz, 1H) 7.16 (t, J=7.25 Hz, 1H) 7.08 (d, J=9.01 Hz, 2H) 6.85-6.92 (m, 1H) 6.52 (t, J=7.44 Hz, 1H) 6.45 (d, J=8.38 Hz, 1H) 5.28 (br s, 1H) 2.93 (br s, 4H) 2.65-2.71 (m, 2H) 2.35 (s, 3H) 2.33 (d, J=1.75 Hz, 2H) 1.66 (d, J=6.63 Hz, 3H)

### Example 42

### 2-((1-(6-methyl-2-(4-morpholinophenyl)-4-oxo-4H-chromen-8-yl)ethyl)amino)benzoic acid

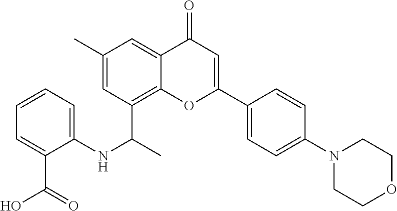 [SMILES: Cc1cc(C(C)Nc2ccccc2C(=O)O)c2oc(-c3ccc(N4CCOCC4)cc3)cc(=O)c2c1]

¹H NMR (400 MHz, DMSO-d₆) δ ppm 12.77 (br s, 1H) 8.44 (br d, J=6.13 Hz, 1H) 7.99 (d, J=9.01 Hz, 2H) 7.82 (dd, J=7.94, 1.56 Hz, 1H) 7.72 (d, J=1.25 Hz, 1H) 7.51 (d, J=1.88 Hz, 1H) 7.18-7.29 (m, 2H) 7.09 (d, J=9.01 Hz, 2H) 6.89-6.92 (m, 1H) 6.47-6.59 (m, 2H) 5.31 (br t, J=6.44 Hz, 1H) 3.73-3.78 (m, 4H) 3.28 (br s, 4H) 2.35 (s, 3H) 1.67 (d, J=6.63 Hz, 3H) MS m/z (ESI): 485 [M+H].

### Example 43

### 2-((1-(2-(4-(4,4-difluoropiperidin-1-yl)phenyl)-6-methyl-4-oxo-4H-chromen-8-yl)ethyl)amino)benzoic acid

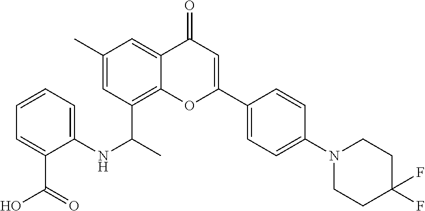 [SMILES: Cc1cc(C(C)Nc2ccccc2C(=O)O)c2oc(-c3ccc(N4CCC(F)(F)CC4)cc3)cc(=O)c2c1]

¹H NMR (400 MHz, ACETONITRILE-d₃) δ ppm 1.65-1.73 (m, 3H) 1.99-2.13 (m, 4H) 2.33-2.40 (m, 3H) 3.55-3.59 (m, 4H) 5.33 (br t, J=6.19 Hz, 1H) 6.49-6.61 (n 2H) 6.93 (s, 1H) 7.18 (br d, J=9.01 Hz, 2H) 7.22-7.28 (m, 1H) 7.54 (d, J=1.75 Hz, 1H) 7.74 (s, 1H) 7.80-7.87 (m, 1H) 8.01 (d, J=8.88 Hz, 2H) 8.48 (br d, J=5.88 Hz, 1H) 12.51-13.06 (m, 1H)

### MS m/z (ESI): 519 [M+H]⁺.

### Example 44

### -((1-(2-(2-isopropyl-1-oxoisoindolin-5-yl)-6-methyl-4-oxo-4H-chromen-8-yl)ethyl)amino)benzoic acid

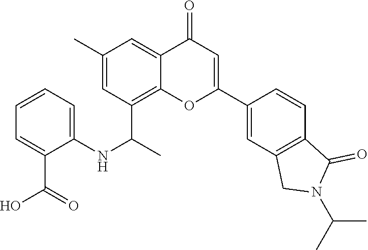 [SMILES: Cc1cc(C(C)Nc2ccccc2C(=O)O)c2oc(-c3ccc4c(c3)CN(C(C)C)C4=O)cc(=O)c2c1]

¹H NMR (400 MHz, DMSO-d₆) δ ppm 12.37-13.21 (m, 1H) 8.44 (br s, 1H) 8.35 (s, 1H) 8.23 (d, J=7.88 Hz, 1H) 7.79-7.88 (m, 2H) 7.76 (s, 1H) 7.60 (br d, J=1.75 Hz, 1H) 7.20-7.29 (i, 1H) 7.17 (s, 1H) 6.56 (br d, J=5.63 Hz, 2H) 5.37 (br d, J=4.88 Hz, 1H) 4.49-4.56 (m, 2H) 4.39-4.47 (n, 1H) 2.38 (s, 3H) 1.68 (br d, J=6.00 Hz, 3H) 1.26 (dd, J=6.75, 2.50 Hz, 6H) MS m/z (ESI): 497 [M+H]⁺.

### Example 45

### 2-((1-(6-methyl-2-(2-(oxetan-3-yl)-1-oxoisoindolin-5-yl)-4-oxo-411-chromen-8-yl)ethyl)amino)benzoic acid

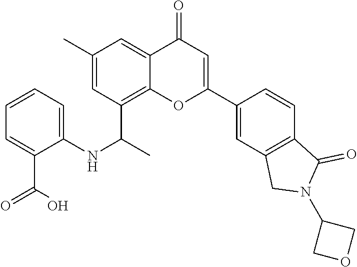 [SMILES: Cc1cc(C(C)Nc2ccccc2C(=O)O)c2oc(-c3ccc4c(c3)CN(C3COC3)C4=O)cc(=O)c2c1]

¹H NMR (400 MHz, DMSO-d₆) δ ppm 12.69-12.96 (m, 1H) 8.44-8.50 (m, 1H) 8.26 (br d, J=8.25 Hz, 1H) 7.84 (br t, J=7.38 Hz, 2H) 7.77 (s, 1H) 7.60 (s, 1H) 7.21-7.27 (m, 2H) 6.50-6.60 (m, 2H) 5.36-5.47 (m, 2H) 4.81-4.92 (m, 6H) 2.38 (s, 3H) 1.69 (br d, J=5.63 Hz, 3H) MS m/z (ESI): 511 [M+H]⁺.

### Example 46

### 2-((1-(2-(2-benzyl-1-oxoisoindolin-5-yl)-6-methyl-4-oxo-4H-chromen-8-yl)ethyl)amino)benzoic acid

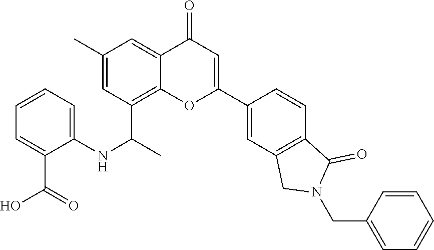 [SMILES: Cc1cc(C(C)Nc2ccccc2C(=O)O)c2oc(-c3ccc4c(c3)CN(Cc3ccccc3)C4=O)cc(=O)c2c1]

¹H NMR (400 MHz, DMSO-d₆) δ ppm 8.32 (s, 1H) 8.25 (br d, J=7.28 Hz, 1H) 7.88 (d, J=7.78 Hz, 1H) 7.82 (br s, 1H) 7.75 (s, 1H) 7.59 (s, 1H) 7.37 (br d, J=7.28 Hz, H) 7.28-7.34 (m, 3H) 7.15 (s, 2H) 6.50 (br d, J=5.27 Hz, 2H) 5.34 (br s, 1H) 4.77 (br s, 2H) 4.46 (s, 2H) 2.37 (s, 3H) 1.65 (br d, J=6.53 Hz, 3H)

MS m/z (ESI): 545 [M+H]⁺.

### Example 47

### 2-((1-(6-methyl-2-(2-methyl-1-oxo-1,2,3,4-tetrahydroisoquinolin-6-yl)-4-oxo-4H-chromen-8-yl)ethyl)amino)benzoic acid

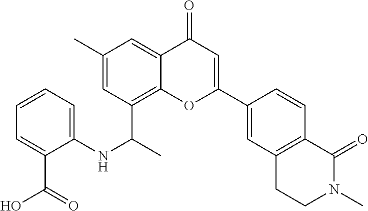 [SMILES: Cc1cc(C(C)Nc2ccccc2C(=O)O)c2oc(-c3ccc4c(c3)CCN(C)C4=O)cc(=O)c2c1]

¹H NMR (400 MHz, DMSO-d₆) δ ppm 12.77 (br s, 1H) 8.42 (br d, J=5.88 Hz, 1H) 8.07-8.12 (n, 1H) 8.04 (br d, J=10.26 Hz, H) 7.82 (br d, J=7.00 Hz, 1H) 7.77 (s, 1H) 7.60 (s, 1H) 7.24 (br t, J=7.13 Hz, 1H) 7.14 (s, 1H) 6.56 (br d, J=8.00 Hz, 2H) 5.36 (br t, J=6.32 Hz, 1H) 3.61 (br t, J=6.50 Hz, 2H) 3.08-3.12 (m, 2H) 3.06 (s, 3H) 2.38 (s, 3H) 1.69 (br d, J=6.50 Hz, 3H) MS m/z (ESI): 483 [M+H]⁺.

### Example 48

### 2-((1-(2-(4-(dimethylcarbamoyl)-3-methylphenyl)-6-methyl-4-oxo-4H-chromen-8-yl)ethyl)amino)benzoic acid

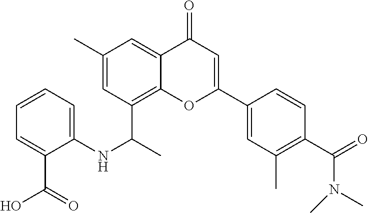 [SMILES: Cc1cc(C(C)Nc2ccccc2C(=O)O)c2oc(-c3ccc(C(=O)N(C)C)c(C)c3)cc(=O)c2c1]

¹H NMR (400 MHz, DMSO-d₆) δ ppm 12.33-13.16 (m, 1H) 8.43 (br d, J=6.00 Hz, 1H) 7.95-8.07 (m, 2H) 7.79-7.85 (m, 1H) 7.76 (d, J=1.38 Hz, 1H) 7.58 (d, J=2.00 Hz, 1H) 7.36 (d, J=8.00 Hz, 1H) 7.24 (s, 1H) 7.12 (s, 1H) 6.50-6.60 (m, 2H) 5.35 (br t, J=6.32 Hz, 1H) 3.03 (s, 3H) 2.78 (s, 3H) 2.37 (s, 3H) 2.9 (s, 3H) 1.69 (d, J=6.50 Hz, 3H) MS m/z (ESI): 485 [M+1-1]

### Example 49

### 2-((1-(3,6-di methyl-2-(2-methyl-2-indazol-5-yl)-4-oxo-4H-chromen-8-yl)ethyl)amino)benzoic acid

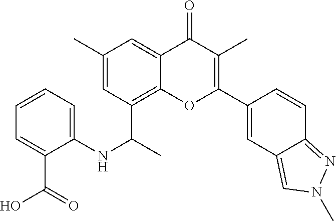 [SMILES: Cc1cc(C(C)Nc2ccccc2C(=O)O)c2oc(-c3ccc4nn(C)cc4c3)c(C)c(=O)c2c1]

The stereoisomers of example 49: (R)-2-((1-(3,6-dimethyl-2-(2-methyl-211-indazol-5-yl)-4-oxo-4H-chromen-8-yl)ethyl)amino)benzoic acid and(S)-2-((1-(3,6-dimethyl-2-(2-methyl-2H-indazol-5-yl)-4-oxo-4H-chromen-8-yl)ethyl)amino)benzoic acid

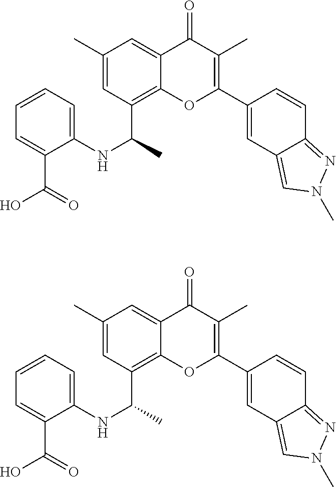 [SMILES: Cc1cc([C@@H](C)Nc2ccccc2C(=O)O)c2oc(-c3ccc4nn(C)cc4c3)c(C)c(=O)c2c1 ; Cc1cc([C@H](C)Nc2ccccc2C(=O)O)c2oc(-c3ccc4nn(C)cc4c3)c(C)c(=O)c2c1]

The stereoisomers were prepared in accordance with the method of Example 20 by chiral SFC ((column: DAICEL CHIRALPAK AD (250 mm*30 mm, 10 um); mobile phase: [0.1% NH3H2O-EtOH], B %: 40%-40%, min) to give Peak 1 (Rt=669) and Peak 2 (Rt=2.217)).

Peak 1: MS m/z (ESI): 468 [M+H]⁺.

¹H NMR (400 MHz, DMSO-d₆) δ ppm 12.78 (br s, 1H), 8.55 (s, 1H), 8.40 (br s, 1H), 8.22 (s, 1H), 7.83-7.75 (m, 3H), 7.62 (d, J=9.2 Hz, 1H), 7.53 (d, J=20 Hz, 1H), 7.21-7.19 (m, 1H), 6.57-6.47 (m, 2H), 5.16-5.13 (m, 1H), 4.23 (s, 3H), 2.37 (s, 3H), 2.14 (s, 3H), 1.60 (d, J=6.4 Hz, 3H).

Peak 2: MS m/z (ESI): 468 [M+H]⁺.

¹H NMR (400 MHz, DMSO-d₆) δ ppm 12.76 (br s, 1H), 8.55 (s, 1H), 8.38 (br s, 1H), 8.22 (s, 1H), 7.83-7.75 (m, 3H), 7.62 (d, J=9.2 Hz, 1H), 7.53 (d, J=2.0 Hz, 1H), 7.21-7.19 (m, 1H), 6.57-6.47 (m, 2H), 5.16-5.13 (m, 1H), 4.23 (s, 3H), 2.37 (s, 3H), 2.14 (s, 3H), 1.60 (d, J=6.4 Hz, 3H).

### Example 50

### 2-((1-(6-methyl-2-(1-methyl-6-oxo-1,6-dihydropyridin-3-yl)-4-oxo-4H-chromen-8-yl)ethyl)amino)benzoic acid

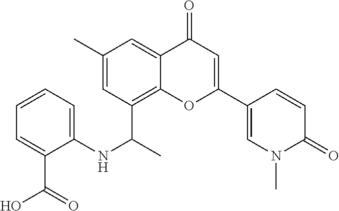 [SMILES: Cc1cc(C(C)Nc2ccccc2C(=O)O)c2oc(-c3ccc(=O)n(C)c3)cc(=O)c2c1]

¹H NMR (400 MHz, DMSO-d₆) δ ppm 12.52-13.01 (m, 1H) 8.59 (d, J=2.50 Hz, 1H) 8.40 (br d, J=5.25 Hz, 1H) 8.08 (dd, J=9.63, 2.75 Hz, 1H) 7.79-7.84 (m, 1H) 7.71 (s, 1H) 7.53 (br d, J=1.63 Hz, 1H) 7.24 (br t, J=7.25 Hz, 1H) 6.87 (s, 1H) 6.47-6.61 (m, 3H) 5.37 (br t, J=6.32 Hz, 1H) 3.56 (s, 3H) 2.35 (s, 3H) 1.64 (d, J=6.63 Hz, 3H) MS m/z (ESI): 431 [M+H]⁺.

### Example 51

### 2-((1-(6-methyl-2-(2-methyl-211-indazol-6-yl)-4-oxo-4H-chromen-8-yl)ethyl)amino)benzoic acid

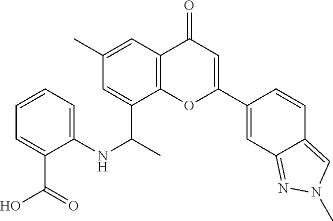 [SMILES: Cc1cc(C(C)Nc2ccccc2C(=O)O)c2oc(-c3ccc4cn(C)nc4c3)cc(=O)c2c1]

¹H NMR (400 MHz, DMSO-d₆) δ ppm 8.46 (br d, J=2.13 Hz, 1H) 7.84-7.91 (m, 1H) 7.70-7.75 (m. 1H) 7.47-7.59 (m, 2H) 731-7.43 (m, 1H) 7.17-7.26 (m, 1H) 7.10-7.14 (m, 1H) 7.02-7.09 (m, 1H) 6.66 (br s, 1H) 6.40-6.43 (m, 1H) 6.37-6.50 (m, 1H) 4.22 (br s, 3H) 2.34 (br s, 3H) 1.60-1.76 (m, 3H).

MS m/z (ES1): 454 [M+H]⁺.

### Example 52

### 2-((1-(6-methyl-2-(1-methyl-1H-indazol-6-yl)-4-oxo-4H-chromen-8-yl)ethyl)amino)benzoic acid

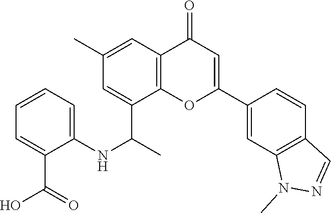 [SMILES: Cc1cc(C(C)Nc2ccccc2C(=O)O)c2oc(-c3ccc4cnn(C)c4c3)cc(=O)c2c1]

¹H NMR (400 MHz, ACETONITRILE-d₃) δ ppm 1.69-1.77 (m, 3H) 2.37 (s, 3H) 4.15 (s, 3H) 5.33-5.49 (m, 1H) 6.53-6.61 (m, 2H) 7.21-7.27 (m, 2H) 7.59 (d, J=1.88 Hz, 1H) 7.77 (d, J=1.13 Hz, 1H) 7.80-7.88 (m, 2H) 7.91-7.96 (m, 1H) 8.17 (s, 1H) 8.41-8.51 (m, 2H) 12.47-12.96 (m, 1H).

MS m/z (ESI): 454 [M+H]⁺.

### Example 53

### 2-((1-(3,6-dimethyl-2-(4-morpholinophenyl)-4-oxo-4H-chromen-8-yl)ethyl)amino)benzoic acid

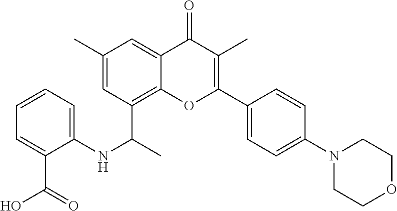 [SMILES: Cc1cc(C(C)Nc2ccccc2C(=O)O)c2oc(-c3ccc(N4CCOCC4)cc3)c(C)c(=O)c2c1]

The stereoisomers of example 53: (R)-2-((1-(3,6-dimethyl-2-(4-morpholinophenyl)-4-oxo-4H-chromen-8-yl)ethyl)amino)benzoic acid and (S)-2-((1-(3,6-dimethyl-2-(4-morpholinophenyl)-4-oxo-4H-chromen-8-yl)ethyl)amino)benzoic acid

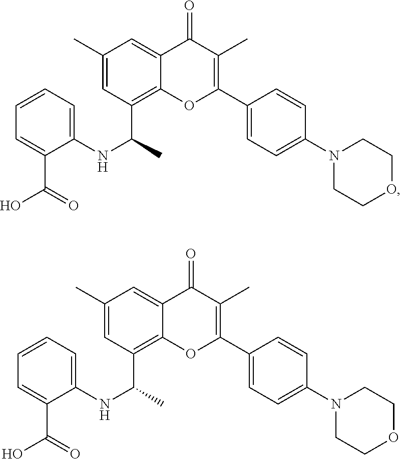 [SMILES: Cc1cc([C@@H](C)Nc2ccccc2C(=O)O)c2oc(-c3ccc(N4CCOCC4)cc3)c(C)c(=O)c2c1 ; Cc1cc([C@H](C)Nc2ccccc2C(=O)O)c2oc(-c3ccc(N4CCOCC4)cc3)c(C)c(=O)c2c1]

The stereoisomers of example 53 were prepared in accordance with the method of Example 20 by chiral SFC (column: DAICEL CHIRALPAK AD-(250 mm*30 mm, 10 um); mobile phase: [0.1% NH3H2O-IPA]; B %: 40%-40%, min) to give Peak 1 (Rt=1.62 min) and Peak 2 (Rt=2.18 min).).

Peak 1: MS m/z (ESI): 499 [M+H]⁺.

¹H NMR (400 MHz, DMSO-d6) δ ppm 8.41 (br s, 1H), 7.80 (br s, 1H), 7.75-7.69 (m, 3H), 7.49 (s, 1H), 7.23-7.19 (m, 1H), 7.10 (d, J=8.0 Hz, 1H), 6.54 (br s, 1H), 6.45 (br s, 1H), 5.15 (br s, 1H), 3.77-3.75 (m, 5H), 3.27 (s, 3H), 2.35 (s, 3H), 2.13 (s, 3H), 1.60 (d, J=5.6 Hz, 3H)

Peak 2: MS mm/z (ESI): 499 [M+H]⁺.

¹H NMR (400 MHz, DMSO)-d6) δ ppm 8.45 (br s, H), 7.80-7.76 (m, 4H), 7.55 (br s, 1H), 7.25-7.16 (m, 3H), 6.60-6.51 (m, 2H), 5.22 (br s, 1H), 3.80 (br s, 5H), 3.27 (br s, 3H), 2.40 (s, 3H), 2.17 (s, 3H), 1.65 (br s, 3H).

### Example 54

### 2-((1-(2-(2-(2-methoxyethyl)-1-oxoisoindolin-5-yl)-6-methyl-4-oxo-4H-chromen-8-yl)ethyl)amino)benzoic acid

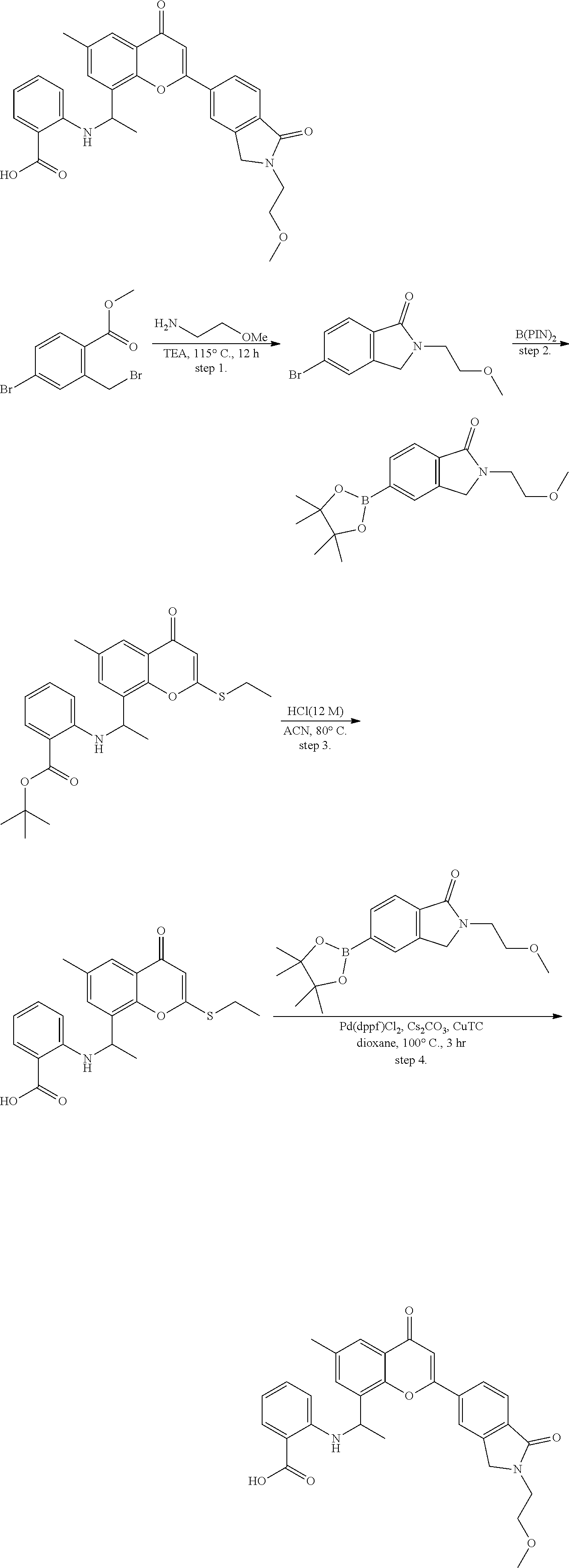

Step 1. To a solution of methyl 4-bromo-2-(bromomethyl)benzoate (1 g, 3.25 mmol, 1 eq), 2-methoxyethanamine (732 mg, 9.74 mmol, 846.83 uL, 3 eq) in toluene (15 mL) was added TEA (985 mg, 9.74 mmol, 1.36 mL, 3 eq). The mixture was stirred at 115° C. for 12 hr. LCMS showed desire d mass was detected The reaction mixture was added with water (30 ml), extracted with EtOAc (50 ml*3) to give the organic phase. Then dried over Na₂SO₄, filtered and concentrated under reduce d pressure to give a residue. The residue was purified by flash silica gel chromatography (ISCO®; 12 g SepaFlash® Silica Flash Column, Eluent of 0-30% Ethyl acetate/Petroleum ether gradient @45 L/min) to give compound 5-bromo-2-(2-methoxyethyl)isoindolin-1-one (800 mg, 2.75 mmol, 84.82% yield, 93% purity) as a white solid.

MS mm/z (ESI): 271 [M+H]⁺.

Step 2. A mixture of 5-bromo-2-(2-methoxyethyl)isoindolin-1-one (400 mg, 1.48 mmol, 1 eq), 4,4,5,5-tetramethyl-2-(4,4,5,5-tetramethyl-1,3,2-dioxaborolan-2-yl)-1,3,2-dioxaborolane (752 mg, 2.96 mmol, 2 eq), KOAc (435 mg, 4.44 mmol, 3 eq), Pd₂(dba)₃ (135 mg, 148.08 μmol, 0.1 eq) and ditert-butyl-[2-(2,4,6-triisopropylphenyl)phenyl]phosphane (62 mg, 148.08 μmol, 0.1 eq) in dioxane (8 mL) was degassed and purged with N₂ for 3 times, and then the mixture was stirred at 90° C. for 12 hr under N₂ atmosphere. LCMS showed desired mass was detected. The reaction mixture was extracted with ethyl acetate (20 ml*3). The combined organic layers were dried over anhydrous Na₂SO₄, filtered and concentrated under reduced pressure to give a crude product. The residue was purified by prep-HPLC (column: Xtimate C18 100*30 mm*10 um; mobile phase: [water (FA)-ACN]; B %: 45%-75%, 10 min). to give compound 2-(2-methoxyethyl)-5-(4,4,5,5-tetramethyl-1,3,2-dioxaborolan-2-yl) isoindolin-1-one (150 mg, 472.91 μmol, 31.94% yield,) as a white solid. MS m/z (ESI): 318 [M+H-]⁺.

### Step 3

To a solution of tert-butyl 2-[I-(2-ethylsulfanyl-6-methyl-4-oxo-chromen-8-yl) ethylamino]benzoate (50 mg, 113.75 μmol, 1 eq) in ACN (2 mL) was added HCl (12 M, 38 μL, 4 eq). The mixture was stirred at 80° C. for 1 hr. LCMS showed desired mass was detected. The reaction mixture was concentrated under vacuum to give the residue. The reaction mixture without purification to give compound 2-[1-(2-ethylsulfanyl-6-methyl-4-oxo-chromen-8-yl) ethylamino]benzoic acid (45 mg, crude) as a white solid.

MS m/z (ESI): 384 [M+H]⁺.

### Step 4

A mixture of 2-[I-(2-ethylsulfanyl-6-methyl-4-oxo-chromen-8-yl)ethylamino]benzoic acid (45 mg, 117.35 μmol, 1 eq), 2-(2-methoxyethyl)-5-(4,4,5,5-tetramethyl-1,3,2-dioxaborolan-2-yl)isoindolin-1-one (41 mg 129.09 μmol, 1.1 eq), Cs₂CO₃ (76 mg, 234.71 μmol, 2 eq), Pd(dppf)Cl₂·CH₂Cl₂ (19 mg, 23.47 μmol, 0.2 eq) and thiophene-2-carbonyloxycopper (45 mg, 234.71 μmol, 2 eq) in dioxane (3 mL) was degassed and purged with N₂ for 3 times, and then the mixture was stirred at 100° C. for 12 hr under N₂ atmosphere. LCMS (EB4733-464-P1E) showed desired mass was detected. The reaction mixture was filtered and the filtration was concentrated under vaccum to give the residue. The residue was purified by prep-H-PLC (column: Xtimate C18 100*30 mm*10 um; mobile phase: [water (FA)-ACN]; B %: 50%-70%, 10 min) to give compound 2-[I-[2-[2-(2-methoxyethyl)-1-oxo-isoindolin-5-yl]-6-methyl-4-oxo-chromen-8-yl]ethylamino]benzoic acid (10 mg, 17.58 μmol, 15% yield, 90% purity) as a white solid.

¹H NMR (400 MHz, DMSO-d₆) δ ppm 12.47-12.93 (m, 1H) 8.43 (br d, J=6.00 Hz, 1H) 8.34 (s, 1H) 8.22 (d, J=8.13 Hz, 1H) 7.82 (d, J=8.00 Hz, 2H) 7.76 (s, 1H) 7.59 (d, J=1.88 Hz, 1H) 7.22-7.27 (m, 1H) 7.16 (s, 1H) 6.54-6.59 (m, 2H) 5.38 (br t, J=6.44 Hz, 1H) 4.61 (s, 2H) 3.70-3.75 (m, 2H) 3.59 (t, J=5.25 Hz, 2H) 3.28 (s, 3H) 2.37 (s, 3H) 1.68 (d, J=6.63 Hz, 3H) MS m/z (ESI): 513 [M+H]⁺.

### Example 55

### 2-((1-(2-(2-(2-(dimethylamino)ethyl)-1-oxoisoindolin-5-yl)-6-methyl-4-oxo-4H-chromen-8-yl)ethyl)amino)benzoic acid

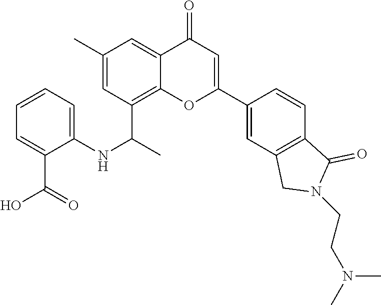 [SMILES: Cc1cc(C(C)Nc2ccccc2C(=O)O)c2oc(-c3ccc4c(c3)CN(CCN(C)C)C4=O)cc(=O)c2c1]

¹H NMR (400 MHz, DMSO-d₆) δ ppm 8.45 (br d, J=5.25 Hz, 1H) 8.37 (s, 1H) 8.24 (br d, J=7.75 Hz, 1H) 7.83 (br t, J=8.38 Hz, 2H) 7.77 (s, 1H) 7.65-7.73 (m, 1H) 7.61 (s, 1H) 7.37 (br s, 1H) 7.21-7.27 (m, 1H) 7.19 (s, 1H) 6.52-6.59 (m, 2H) 5.32-5.40 (m, 1H) 4.63 (br s, 2H) 3.81 (br s, 2H) 2.54-2.62 (m, 6H) 2.38 (s, 3H) 1.68 (br d, J=6.25 Hz, 3H) MS m/z (ESI): 526 [M+H]⁺.

### Example 56

### 2-((1-(6-methyl-2-(2-(oxetan-3-yl)-2H-indazol-5-yl)-4-oxo-4H-chromen-8-yl)ethyl)amino)benzoic acid

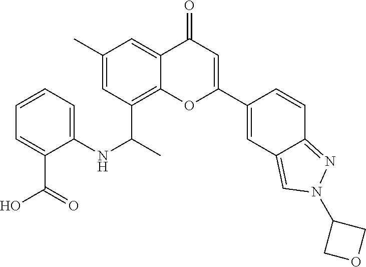 [SMILES: Cc1cc(C(C)Nc2ccccc2C(=O)O)c2oc(-c3ccc4nn(C5COC5)cc4c3)cc(=O)c2c1]

2-((1-(6-methyl-2-(2-(oxetan-3-yl)-2H-indazol-5-yl)-4-oxo-4H-chromen-8-yl)ethyl)amino)benzoic acid was prepared in accordance with the method of Example 54. H NMR (400 MHz, DMSO-d6) δ ppm 8.73 (s, 1H) 8.64 (s, 1H) 8.19 (s, 1H) 7.96 (dd, J=9.19, 1.56 Hz, 1H) 7.79-7.88 (m, 2H) 7.75 (s, 1H) 7.57 (d, J=1.75 Hz, 1H) 7.16-7.23 (m, 1H) 7.08 (s, 1H) 6.49-6.59 (m, 2H) 5.96 (quin, J=6.85 Hz, 1H) 5.30-5.44 (m, 1H) 5.02-5.08 (m, 4H) 2.37 (s, 3H) 1.69 (d, J=6.63 Hz, 3H) MS m/z (ESI): 496 [M+H]⁺.

### Example 57

### 2-((1-(2-(2-(2-hydroxyethyl)-2H-indazol-5-yl)-6-methyl-4-oxo-4H-chromen-8-yl)ethyl)amino)benzoic acid

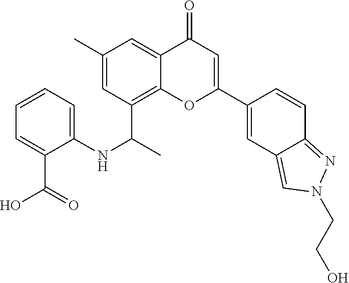 [SMILES: Cc1cc(C(C)Nc2ccccc2C(=O)O)c2oc(-c3ccc4nn(CCO)cc4c3)cc(=O)c2c1]

2-((1-(2-(2-(2-hydroxyethyl)-2H-indazol-5-yl)-6-methyl-4-oxo-4H-chromen-8-yl)ethyl)amino)benzoic acid was prepared in accordance with the method of Example 5′4. H NMR (400 MHz, DMSO-d) δ ppm 8.65 (s, 1H) 8.59 (s, 1H) 7.92 (br d, J=9.26 Hz, 1H) 7.83 (br d, J=7.88 Hz, 1H) 7.72-7.79 (m, 2H) 7.57 (s, 1H) 7.22 (br t, J=7.00 Hz, 1H) 7.06 (s, 1H) 6.49-6.62 (m, 2H) 5.39 (br t, J=6.07 Hz, 1H) 4.89-5.16 (m, 1H) 4.51 (br t, J=5.25 Hz, 2H) 3.83-3.97 (m, 2H) 2.38 (s, 3H) 1.70 (br d, J=6.63 Hz, 3H) MS m/z (ESI): 484[M+H]⁺.

### Example 58

### 2-((1-(2-(2-(2-methoxyethyl)-211-indazol-5-yl)-6-methyl-4-oxo-4H-chromen-8-yl)ethyl)amino)benzoic acid

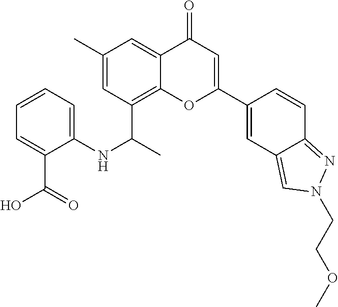 [SMILES: COCCn1cc2cc(-c3cc(=O)c4cc(C)cc(C(C)Nc5ccccc5C(=O)O)c4o3)ccc2n1]

2-((1-(2-(2-(2-methoxyethyl)-2H-indazol-5-yl)-6-methyl-4-oxo-4H-chromen-8-yl)ethyl)amino)benzoic acid was prepared in accordance with the method of Example 54.

¹H NMR (400 MHz, DMSO-d₆) δ ppm 12.78 (dt, J=8.16, 1.67 Hz, 1H) 8.63 (s, 1H) 8.59 (s, 1H) 8.47 (br d, J=5.38 Hz, 1H) 7.92 (br d, J=9.38 Hz, 1H) 7.83 (br d, J=8.13 Hz, 1H) 7.71-7.78 (m, 2H) 7.57 (s, 1H) 7.25 (br t, J=7.88 Hz, 1H) 7.06 (s, 1H) 6.49-6.64 (m, 2H) 5.40 (br t, J=6.32 Hz, 1H) 4.64 (br t, J=4.94 Hz, 2H) 3.86 (br t, J=5.00 Hz, 2H) 3.25 (s, 3H) 2.38 (s, 3H) 1.71 (br d, J=6.50 Hz, 3H)

MS m/z (ESI): 498 [M+H]⁺.

### Example 59

### 2-((1-(2-(1,2-dimethyl-1H-benzo[d]imidazol-6-yl)-6-methyl-4-oxo-411-chromen-8-yl)ethyl)amino)benzoic acid

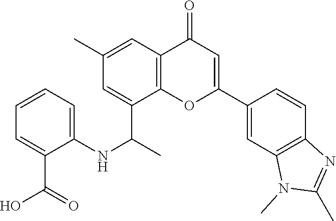 [SMILES: Cc1cc(C(C)Nc2ccccc2C(=O)O)c2oc(-c3ccc4nc(C)n(C)c4c3)cc(=O)c2c1]

2-((1-(2-(1,2-dimethyl-1H-benzo[d]imidazol-6-yl)-6-methyl-4-oxo-4H-chromen-8-yl)ethyl)amino)benzoic acid was prepared in accordance with the method of Example 1.

¹H NMR (400 MHz, DMSO-d₆) δ ppm 8.61 (br s, 1H) 8.44 (br s, 1H) 8.22 (br d, J=8.50 Hz, 1H) 7.90 (br d, J=8.63 Hz, 1H) 7.80-7.85 (m, 1H) 7.78 (br s, 1H) 7.59 (br s, 1H) 7.28-7.31 (m, 1H) 7.21-7.26 (m, 1H) 6.54-6.59 (m, 2H) 5.40 (br d, J=5.00 Hz 1H) 3.98 (br s, 3H) 2.81 (br s, 3H) 2.39 (s, 3H) 1.72 (br d, J=6.38 Hz, 3H) MS m/z (ESI): 468 [M+H]⁺.

### Example 60

### 2-((1-(6-methyl-2-(1-methyl-1H-benzo[d]imidazol-6-yl)-4-oxo-4H-chromen-8-yl)ethyl)amino)benzoic acid

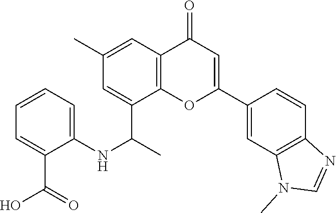 [SMILES: Cc1cc(C(C)Nc2ccccc2C(=O)O)c2oc(-c3ccc4ncn(C)c4c3)cc(=O)c2c1]

2-((1-(6-methyl-2-(1-methyl-1H-benzo[d]imidazol-6-yl)-4-oxo-4H-chromen-8-yl)ethyl)amino)benzoic acid was prepared in accordance with the method of Example 1. ¹H NMR (400 MHz, DMSO-d₆) δ ppm 12.44-13.02 (m, 1H) 8.78 (s, 1H) 8.47 (br d, J=6.38 Hz, 1H) 8.04-8.20 (m, 2H) 7.82 (br d, J=8.00 Hz, 1H) 7.77 (br s, 1H) 7.57 (br s, 1H) 7.18-7.29 (m, 2H) 6.57 (br d, J=5.50 Hz, 2H) 5.35-5.48 (m, 1H) 4.58 (s, 3H) 2.38 (s, 3H) 1.72 (br d, J=6.25 Hz, 3H)

MS m/z (ESI): 455 [M+H]⁺.

### Example 61

### 2-((1-(2-(2,6-dimethyl-21-indazol-5-yl)-6-methyl-4-oxo-4H-chromen-8-yl)ethyl)amino)benzoic acid

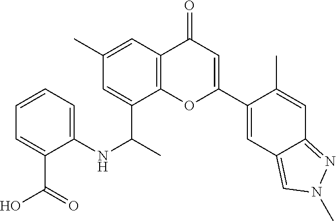 [SMILES: Cc1cc(C(C)Nc2ccccc2C(=O)O)c2oc(-c3cc4cn(C)nc4cc3C)cc(=O)c2c1]

2-((1-(2-(2,6-dimethyl-2H-indazol-5-yl)-6-methyl-4-oxo-41-H-chromen-8-yl)ethyl)amino)benzoic acid was prepared in accordance with the method of Example 1.

¹H NMR (400 MHz, DMSO-d₆) δ ppm 12.79 (br s, 1H) 8.26-8.43 (m, 1H) 7.69-7.93 (m, 2H) 7.69-7.93 (m, 1H) 7.55 (br d, J=12.01 Hz, 2H) 7.07-7.24 (mi, 2H) 6.86 (s, 1H) 6.40-6.61 (m, 21H) 5.18-5.33 (m, 1H) 4.48 (s, 3H) 2.35-2.45 (m, 6H) 1.61 (br d, J=6.13 Hz, 3H) MS m/z (ESI): 468 [M+H]⁺.

### Example 62

### 2-((1-(2-(2,4-dimethyl-2H-indazol-5-yl)-6-methyl-4-oxo-4H-chromen-8-yl)ethyl)amino)benzoic acid

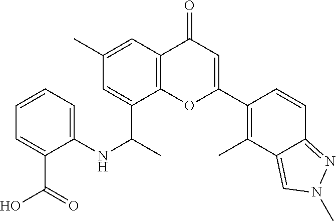 [SMILES: Cc1cc(C(C)Nc2ccccc2C(=O)O)c2oc(-c3ccc4nn(C)cc4c3C)cc(=O)c2c1]

2-((1-(2-(2,4-dimethyl-2H-indazol-5-yl)-6-methyl-4-oxo-4H-chromen-8-yl)ethyl)amino)benzoic acid was prepared in accordance with the method of Example 1.

¹H NMR (400 MHz, ACETONITRILE-d₃) δ ppm 8.65 (s, 1H) 8.39 (br d, J=2.00 Hz, 1H) 7.75-7.86 (m, 2H) 7.48-7.61 (m, 4H) 7.15-7.23 (m, 1H) 6.51-6.58 (m, 2H) 6.43 (d, J=8.50 Hz, 1H) 5.12-5.22 (m, 1H) 4.21 (s, 3H) 2.73 (s, 3H) 2.37 (s, 3H) 1.55-1.62 (m, 3H)

MS m/z (ESI): 468 [M+H]⁺.

### Example 63

### 2-((1-(6-methyl-2-(3-methyl-2-oxo-2,3-dihydrobenzo[d]oxazol-5-yl)-4-oxo-5,6-dihydro-4H-chromen-8-yl)ethyl)amino)benzoic acid

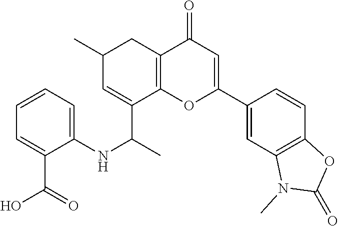 [SMILES: CC1C=C(C(C)Nc2ccccc2C(=O)O)c2oc(-c3ccc4oc(=O)n(C)c4c3)cc(=O)c2C1]

2-((1-(6-methyl-2-(3-methyl-2-oxo-2,3-dihydrobenzo[d]oxazol-5-yl)-4-oxo-5,6-dihydro-4H-chromen-8-yl)ethyl)amino)benzoic acid was prepared in accordance with the method of Example 1.

¹H NMR (400 MHz, DMSO-d6) δ ppm 12.49-12.98 (m, 1H) 8.43 (br d, J=5.38 Hz, 1H) 8.01 (s, 1H) 7.94 (br d, 1=8 0.38 Hz, 1H) 7.81 (br d, J==7.63 Hz, 1H) 7.74 (br s, 1H) 7.57 (br s, 1H) 7.50 (br d, J=8.38 Hz, 1H) 7.23 (br t, J=7.44 Hz, 1H) 7.16 (s, 1H) 6.51-6.57 (m, 2H) 5.32-5.38 (M, 1H) 3.42 (br s, 31H) 2.36 (s, 3H) 1.68 (br d, J=6.38 Hz, 3H) MS m/z (ESI): 473 [M+H]⁺.

### Example 64

### 2-((1-(6-methyl-4-oxo-2-(4-(3-oxomorpholino)phenyl)-4H-chromen-8-yl)ethyl)amino)benzoic acid

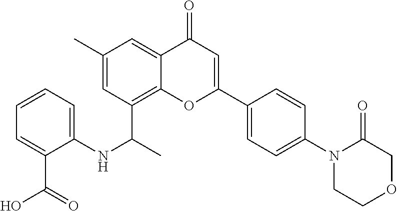 [SMILES: Cc1cc(C(C)Nc2ccccc2C(=O)O)c2oc(-c3ccc(N4CCOCC4=O)cc3)cc(=O)c2c1]

2-((1-(6-methyl-4-oxo-2-(4-(3-oxomorpholino)phenyl)-4H-chromen-8-yl)ethyl)amino)benzoic acid was prepared in accordance with the method of Example 1.

¹H NMR (400 MHz, ACETONITRILE-d₃) δ ppm 8.45-8.52 (m, 1H) 8.17 (d, J=8.76 Hz, 2H) 7.80-7.84 (m, 1H) 7.75 (d, J=1.25 Hz, 1H) 7.66 (d, 1=8.88 Hz, 2H) 7.56 (d, J=2.00 Hz, 1H) 7.22 (t, J=7.07 Hz, 1H) 7.09 (s, 1H) 6.50-6.57 (m, 2H) 5.27-5.37 (m, 1H) 4.26 (s, 2H) 3.98-4.04 (m, 2H) 3.81-3.86 (n, 2H) 2.36 (s, 3H) 1.66-1.69 (m, 3H) MS m/z (ES1): 499 [M+H]⁺.

### Example 65

### 2-((1-(2-(4-(4-acetylpiperazin-1-yl)phenyl)-6-methyl-4-oxo-4H-chromen-8-yl)ethyl)amino)benzoic acid

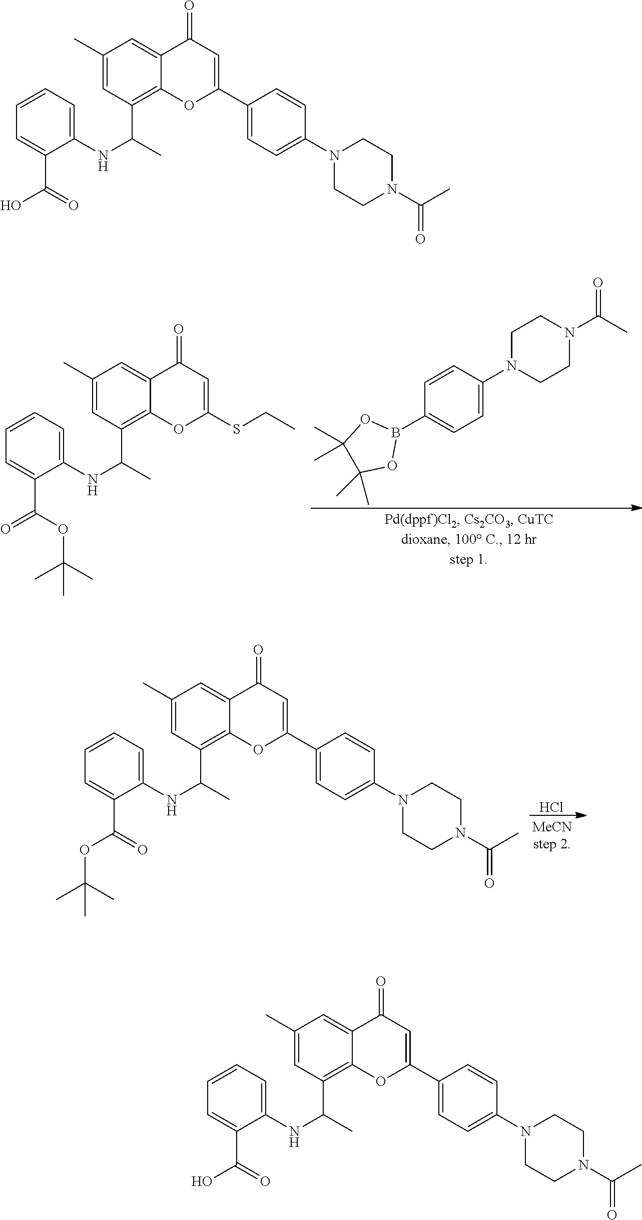

### Step 1

To a solution of tert-butyl 2-[1-(2-ethylsulfanyl-6-methyl-4-oxo-chromen-8-yl) ethylamino]benzoate (150 mg, 341.25 μmol, 1 eq), 1-[4-[4-(4,4,5,5-tetramethyl-1,3,2-dioxaborolan-2-yl)phenyl]piperazin-1-yl] ethanone (225 mg, 682.49 μmol, 2 eq), Cs₂CO₃ (222 mg, 682.49 μmol, 2 eq), Pd(dppf)Cl₂·CHCl₂ (55 mg, 68.25 μmol, 0.2 eq) and thiophene-2-carbonyloxycopper (130 mg, 682.49 μmol, 2 eq) in dioxane (5 mL) was degassed and purged with N₂ for 3 times, and then the mixture was stirred at 100° C. for 12 hr under N₂ atmosphere. The reaction mixture was extracted with ethyl acetate (20 ml*³). The combined organic layers were dried over anhydrous Na₂SO₄, filtered and concentrated under reduced pressure to give a crude product. The residue was purified by flash silica gel chromatography (ISCO®; 4 g SepaFlash™ Silica Flash Column, Eluent of 0˜-100% Ethyl acetate/Petroleum ether gradient @ 20 mL/min). Compound tert-butyl 2-[1-[2-[4-(4-acetylpiperazin-1-yl)phenyl]-6-methyl-4-oxo-chromen-8-yl]ethylamino]benzoate (150 mg, 234.66 μmol, 68% yield, 91% purity) was obtained as a white solid.

MS m/z (ESI): 582 [M+H]⁺.

### Step 2

To a solution of tert-butyl 2-[1-[2-[4-(4-acetylpiperazin-1-yl)phenyl]-6-methyl-4-oxo-chromen-8-yl]ethylamino]benzoate (100 mg, 171.91 μmol, 1 eq) in ACN (3 mL) was added HCl (12 M, 57.30 uL, 4 eq). The mixture was stirred at 80° C. for 1 hr. The reaction mixture was filtered and the filtration was concentrated under vacuum to give the residue. The residue was purified by prep-HPLC (column: Xtimate C18 100*30 mm*10 um; mobile phase: [water (FA)-ACN]; B %: 50%-80%, 10 min) to give desired compound (70 mg, purity 98%) as a yellow solid.

MS m/z (ESI): 526 [M+H]⁺.

The stereoisomers of example 170: (S)-2-((1-(2-(4-(4-acetylpiperazin-1-yl)phenyl)-6-methyl-4-oxo-4H-chromen-8-yl)ethyl)amino)benzoic acid and (R)-2-((1-(2-(4-(4-acetylpiperazin-1-yl)phenyl)-6-methyl-4-oxo-4H-chromen-8-yl)ethyl)amino)benzoic acid

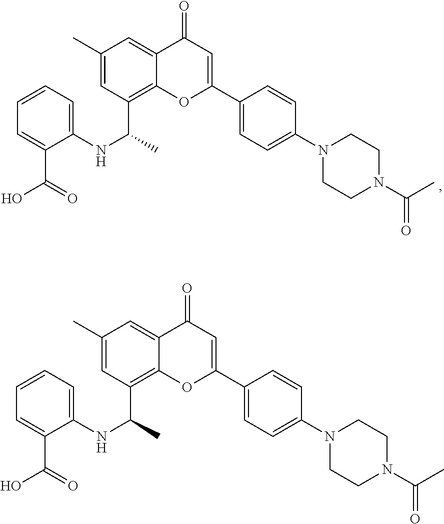 [SMILES: CC(=O)N1CCN(c2ccc(-c3cc(=O)c4cc(C)cc([C@H](C)Nc5ccccc5C(=O)O)c4o3)cc2)CC1 ; CC(=O)N1CCN(c2ccc(-c3cc(=O)c4cc(C)cc([C@@H](C)Nc5ccccc5C(=O)O)c4o3)cc2)CC1]

The stereoisomers of example 65 were prepared in accordance with the method of Example 20 by chiral SFC (c column: Daicel Chiralpak IBN 250 mm*30 mm*1 0 um; mobile phase: [Neu-MeOH]; B %: 60%-60%, min) to give Peak 1 (Rt==1.29 min) and Peak 2 (Rt=1.98 min).

Peak 1: MS m/z (ESI): 526 [M+H]⁺.

¹H NMR (400 MHz, DMSO-d₆) δ ppm 9.40-9.55 (m, 1H) 7.97 (br d, J=8.63 Hz, 2H) 7.90 (br d, J=7.00 Hz, 1H) 7.68 (s, 1H) 7.49 (s, 1H) 7.08 (br d, J=8.76 Hz, 2H) 7.01 (br t, 1=7.57 Hz, 1H) 6.88 (s, 1H) 6.45 (br t, J=7.38 Hz, 1H) 6.30 (br d, J=8.13 Hz, 1H) 5.24 (br t, J=6.32 Hz, 1H) 3.58 (br s, 4H) 3.32 (br d, J=4.88 Hz, 4H) 2.30 (br s, 3H) 2.04 (s, 3H) 1.60 (br d, J=6.38 Hz, 3H)

Peak 2: MS m/z (ESI): 526 [M+H]⁺.

¹H NMR (400 MHz, DMSO-d₆) δ ppm 9.22-9.37 (m, 1H) 7.99 (br d, J=8.76 Hz, 2H) 7.88 (br d, J=7.50 Hz, 1H) 7.70 (s, 1H) 7.51 (s, 1H) 7.10 (br d, J=8.88 Hz, 2H) 702-7.06 (m, 1H) 6.90 (s, 1H) 6.47 (br t, J=7.32 Hz, 1H) 6.35 (br d, J=8.25 Hz, 1H) 5.26 (br s, 1H) 3.59 (br s, 4H) 3.32-3.34 (m, 4H) 2.33 (s, 3H) 2.05 (s, 3H) 1.63 (br d, J=6.50 Hz, 3H)

### Example 66

### 2-((1-(6-methyl-2-(4-(3-methylmorpholino)phenyl)-4-oxo-4H-chromen-8-yl)ethyl)amino)benzoic acid

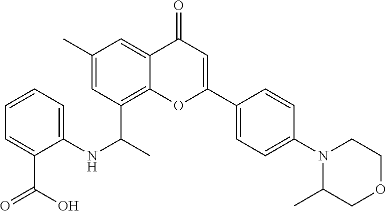 [SMILES: Cc1cc(C(C)Nc2ccccc2C(=O)O)c2oc(-c3ccc(N4CCOCC4C)cc3)cc(=O)c2c1]

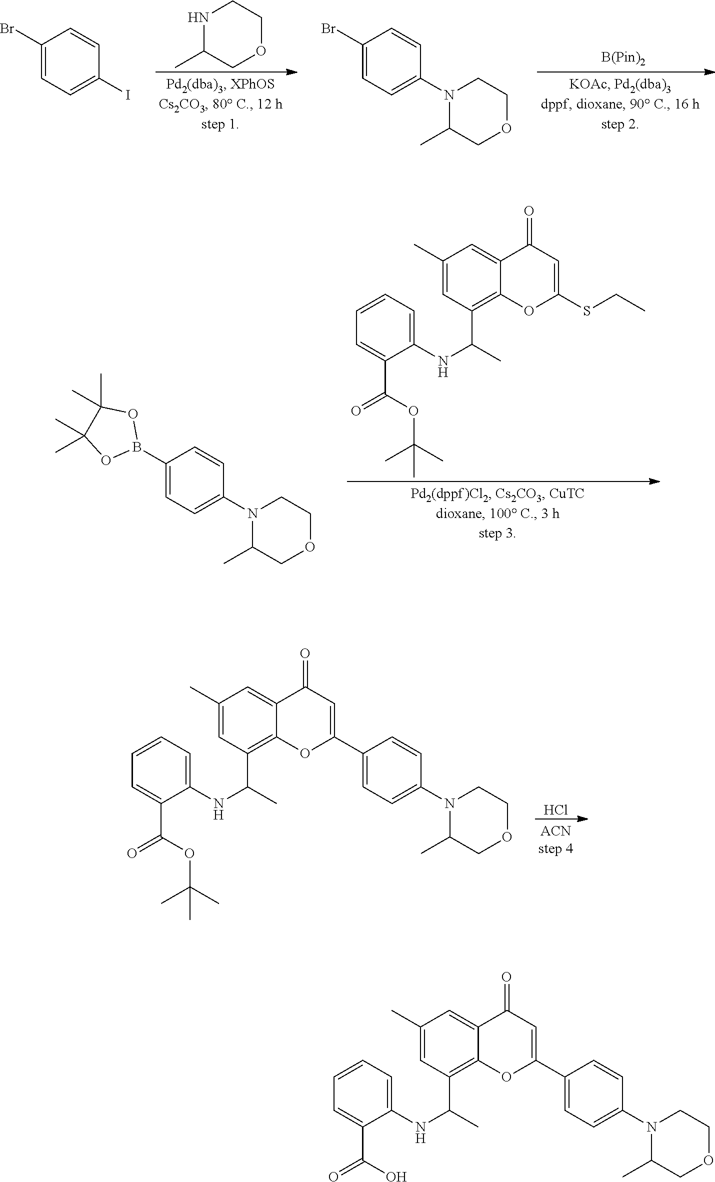

### Step 1

A mixture of 1-bromo-4-iodo-benzene (1 g, 3.53 mmol, 1 eq), 3-methylmorpholine (3570.5 mg, 3.53 mmol, 1 eq), Pd₂(dba)₃ (324 mg, 353.48 μmol, 0.1 eq), Cs₂CO₃ (3.5 g, 10.60 mmol, 3 eq) and (5-diphenylphosphanyl-9,9-dimethyl-xanthen-4-yl)-diphenyl-phosphane (204.5 mg, 353.48 μmol, 0.1 eq) in dioxane (30 mL) was degassed and purged with N₂ for 3 times, and then the mixture was stirred at 80° C. for 12 hr under N₂ atmosphere. LCMS showed desired mass was detected. The reaction mixture was extracted with ethyl acetate (50 ml*3). The combined organic layers were dried over anhydrous Na₂SO₄, filtered and concentrated under reduced pressure to give a crude product. The residue was purified by flash silica gel chromatography (ISCO®; 10 g SepaFlash® Silica Flash Column, Eluent of 0-10% Ethyl acetate/Petroleum ether gradient @, 40 mL/min) to give compound 4-(4-bromophenyl)-3-methyl-morpholine (500 mg, 1.95 mmol, 55% yield,) as a white solid.

MS m/z (ESI): 258 [M+H]⁺.

### Step 2

A mixture of 4-(4-bromophenyl)-3-methyl-morpholine (300 mg. 1.17 mmol, 1 eq), 4,4,5,5-tetramethyl-2-(4,4,5,5-tetramethyl-1,3,2-dioxaborolan-2-yl)-1,3,2-dioxaborolane (595 g, 2.34 mmol, 2 eq), KOAc (345 mg. 3.51 mmol, 3 eq), Pd₂(dba)₃ (107 mg, 117.12 μmol, 0.1 eq) and i-tert-butyl-[2-(2,4,6-triisopropylphenyl)phenyl]phosphane (49 mg, 117.12 μmol, 0.1 eq) in dioxane (10 mL) was degassed and purged with N₂ for 3 times, and then the mixture was stirred at 90° C. for 12 hr under N₂ atmosphere. LCMS showed desired mass was detected. The reaction mixture was extracted with ethyl acetate (20 ml*3). The combined organic layers were dried over anhydrous Na₂SO₄ filtered and concentrated under reduced pressure to give a crude product. The residue was purified by prep-HPLC (column: Welch Xtimate C18 150*30 mm*5 um; mobile phase: [water (HCl)-ACN]; B %: 41%-71%, 10 min) to give compound 3-methyl-4-[4-(4,4,5,5-tetramethyl-1,3,2-dioxaborolan-2-yl)phenyl]morpholine (150 ng, 494.72 μmol, 42% yield,) as a white solid.

MS m/z (ESI): 304 [M+H]⁺.

### Step 3

A mixture of tert-butyl 2-[1-(2-ethylsulfanyl-6-methyl-4-oxo-chromen-8-yl) ethylamino]benzoate (50 mg, 113.75 μmol, 1 eq), 3-methyl-4-[4-(4,4,5,5-tetramethyl-1,3,2-dioxaborolan-2-yl)phenyl]morpholine (69 mg, 227.50 μmol, 2 eq), Cs₂CO₃ (74 mg, 227.50 μmol, 2 eq), Pd(dppf)Cl₂·CH2Cl₂ (18 mg, 22.75 μmol, 0.2 eq) and thiophene-2-carbonyloxycopper (43 mg, 227.50 μmol, 2 eq) in dioxane (3 mL) was degassed and purged with N₂ for 3 times, and then the mixture was stirred at 100° C. for 3 hr under N₂ atmosphere. LCMS showed desired mass was detected. The reaction mixture was extracted with ethyl acetate (10 ml*3). The combined organic layers were dried over anhydrous Na₂SO₄ filtered and concentrated under reduced pressure to give a crude product. The residue was purified by prep-TLC (SiO₂, Petroleum ether:Ethyl acetate=2:1) to give compound tert-butyl 2-[1-[6-methyl-2-[4-(3-methylmorpholin-4-yl)phenyl]-4-oxo-chromen-8-yl]ethylamino]benzoate (40 mg, 64.90 μmol, 57% yield, 90% purity) as a white solid.

MS m/z (ESI): 551 [M+H]⁺.

### Step 44

To a solution of tert-butyl 2-[1-[6-methyl-2-[4-(3-methylmorpholin-4-yl) phenyl]-4-oxo-chromen-8-yl]ethylamino]benzoate (40 mg, 72.11 μmol, 1 eq) in ACN (1 mL) was added HCl (12 M, 24.04 uL, 4 eq). The mixture was stirred at 80° C. for 1 hr. LC MS showed desired mass was detected. The reaction mixture was filtered and the filtration was concentrated under vacuum to give the residue. The residue was purified by prep-HPLC (column: Xtimate C18 100*30 mm*10 um; mobile phase [water (FA)-ACN]; 13%: 55%-85%, 10 min) to give compound 2-[1-[6-methyl-2-[4-(3-methylmorpholin-4-yl)phenyl]-4-oxo-chromen-8-yl]ethylamino]benzoic acid (20 mg, 37.39 μmol, 52% yield, 93% purity) as a white solid.

¹H NMR (400 MHz, DMSO-d) δ ppm 12.54-12.94 (m, 1H) 8.43 (br d, J=5.75 Hz, 1H) 7.98 (br d, J=8.88 Hz, 2H) 7.82 (br d, J=7.75 Hz, 1H) 7.71 (s, 1H) 7.51 (s, 1H) 7.23 (br t, J=7.57 Hz, 1H) 7.02 (br d, J=8.88 Hz, 2H) 6.88 (s, 1H) 6.55 (t, J=7.50 Hz, 1H) 6.50 (br d, J=8.25 Hz, 1H) 5.31 (br t, J=6.32 Hz, 1H) 4.08 (br d, J=5.13 Hz, 1H) 3.96 (br d, J=8.76 Hz, 1H) 3.67-3.76 (m, 2H) 3.44-3.58 (m, 2H) 3.04-3.12 (n, 1H) 2.35 (s, 3H) 1.67 (br d, J=6.38 Hz, 3H) 1.09 (br d, J=6.38 Hz, 3H) MS m/z (ESI): 499 [M-+H]⁺.

### Example 67

### 2-((1-(6-methyl-2-(4-(2-methylmorpholino)phenyl)-4-oxo-4H-chromen-8-yl)ethyl)amino)benzoic acid

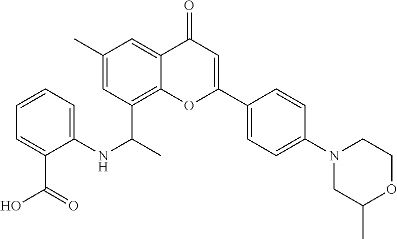 [SMILES: Cc1cc(C(C)Nc2ccccc2C(=O)O)c2oc(-c3ccc(N4CCOC(C)C4)cc3)cc(=O)c2c1]

2-((1-(6-methyl-2-(4-(2-methylmorpholino)phenyl)-4-oxo-4H-chromen-8-yl)ethyl)amino)benzoic acid was prepared in accordance with the method of Example 66. ¹H NMR (400 MHz, ACETONITRILE-d₃) δ ppm 8.41-8.48 (m, 1H) 7.92-8.04 (m, 2H) 7.82 (dd, J=7.94, 1.56 Hz, 1H) 7.72 (d, J=1.38 Hz, 1H) 7.50-7.55 (m, 1H) 7.20-7.27 (m, 1H) 7.04-7.13 (m, 2H) 6.90 (s, 1H) 6.48-6.59 (m, 2H) 5.25-5.36 (m, 1H) 3.89-3.97 (m, 1H) 3.78-3.85 (m, 1H) 3.72 (br d, J=12.38 Hz, 1H) 3.57-3.67 (m, 2H) 3.42 (br s, 1H) 2.76-2.85 (m, 1H) 2.33-2.38 (m, 3H) 1.67 (br d, J=6.63 Hz, 3H) 1.17 (d, J=6.25 Hz, 3H) MS m/z (ESI): 499 [M+H]⁺.

### Example 68

### 2-((1-(2-(4-(2,6-dimethylmorpholino)phenyl)-6-methyl-4-oxo-4H-chromen-8-yl)ethyl)amino)benzoic acid

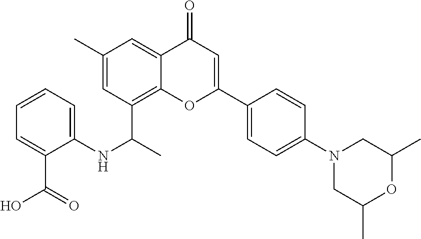 [SMILES: Cc1cc(C(C)Nc2ccccc2C(=O)O)c2oc(-c3ccc(N4CC(C)OC(C)C4)cc3)cc(=O)c2c1]

2-((14(2-(4-(2,6-dimethylmorpholino)phenyl)-6-methyl-4-oxo-4H-chromen-8-yl)ethyl)amino)benzoic acid was prepared in accordance with the method of Example 66.

¹H NMR (400 MHz, DMSO-d₆) δ ppm 8.45 (br d, J=5.88 Hz, 1H) 7.98 (d, J=9.13 Hz, 2H) 7.82 (dd, J=8.07, 1.56 Hz, 1H) 7.71 (s, 1H) 7.51 (d, J=2.00 Hz, 1H) 7.19-7.26 (m, 1H) 7.09 (d, J=9.01 Hz, 2H) 6.90 (s, 1H) 6.47-6.59 (M, 2H) 5.23-5.36 (m, 1H) 3.82 (br d, J=11.76 Hz, 2H) 3.64-3.71 (m, 2H) 2.37-2.41 (m, 2H) 2.35 (s, 3H) 1.67 (d, J==6.63 Hz, 3H) 1.17 (d, J=6.25 Hz, 6H)

MS m/z (ESI): 513 [M+H]⁺.

### Example 69

### 2-((1-(6-methyl-2-(2-methyl-2H-benzo[d][1,2,3]triazol-5-yl)-4-oxo-4H-chromen-8-yl)ethyl)amino)benzoic acid

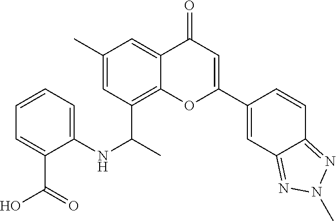 [SMILES: Cc1cc(C(C)Nc2ccccc2C(=O)O)c2oc(-c3ccc4nn(C)nc4c3)cc(=O)c2c1]

2-((1-(6-methyl-2-(2-methyl-21-H-benzo[d][1,2,3]triazol-5-yl)-4-oxo-4H-chromen-8-yl)ethyl)amino)benzoic acid was prepared in accordance with the method of Example 1.

¹H NMR (400 MHz, DMSO-d₆) δ ppm 12.44-13.02 (n, 1H) 8.78 (s, 1H) 8.47 (br d, J=6.38 Hz, 1H) 8.04-8.20 (m, 2H) 7.82 (br d, J=8.00 Hz, 1H) 7.77 (br s, 1H) 7.57 (br s, 1H) 7.18-7.29 (m, 2H) 6.57 (br d, J=5.50 Hz, 2H) 5.35-5.48 (m, 1H) 4.58 (s, 3H) 2.38 (s, 3H) 1.72 (br d, J=6.25 Hz, 3H)

MS m/z (ESI): 455 [M+H]⁺.

### Example 70

### 2-((1-(3,6-dimethyl-2-(2-methyl-1-oxo-1,2,3,4-tetrahydroisoquinolin-6-yl)-4-oxo-4H-chromen-8-yl)ethyl)amino)benzoic acid

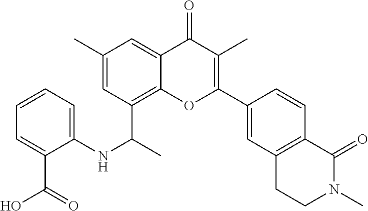 [SMILES: Cc1cc(C(C)Nc2ccccc2C(=O)O)c2oc(-c3ccc4c(c3)CCN(C)C4=O)c(C)c(=O)c2c1]

2-((1-(3,6-dimethyl-2-(2-methyl-1-oxo-1,2,3,4-tetrahydroisoquinolin-6-yl)-4H-oxo-4H-chromen-8-yl)ethyl)amino)benzoic acid is prepared in accordance with the method of Example 1

MS m/z (ESI):497 [M+H]⁺.

### Example 71

### 2-((1-(2-(2-ethyl-1-oxo-1,2,3,4-tetrahydroisoquinolin-6-yl)-3,6-dimethyl-4-oxo-4H-chromen-8-yl)ethyl)amino)benzoic acid

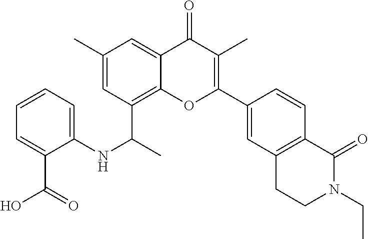 [SMILES: CCN1CCc2cc(-c3oc4c(C(C)Nc5ccccc5C(=O)O)cc(C)cc4c(=O)c3C)ccc2C1=O]

2-((1-(2-(2-ethyl-1-oxo-1,2,3,4-tetrahydroisoquinolin-6-yl)-3,6-dimethyl-4-oxo-4H-chromen-8-yl)ethyl)amino)benzoic acid is prepared in accordance with the method of Example 1.

MS m/z (ESI): 511 [M+H]⁺.

### Example 72

### 2-((1-(3,6-dimethyl-2-(2-methyl-1-oxo-1,2,3,4-tetrahydropyrrolo[1,2-a]pyrazin-7-yl)-4-oxo-4H-chromen-8-yl)ethyl)amino)benzoic acid

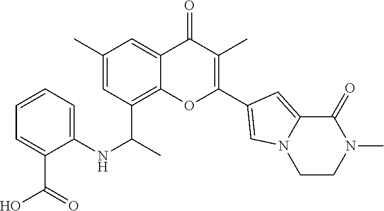 [SMILES: Cc1cc(C(C)Nc2ccccc2C(=O)O)c2oc(-c3cc4n(c3)CCN(C)C4=O)c(C)c(=O)c2c1]

2-((1-(3,6-dimethyl-2-(2-methyl-1-oxo-1,2,3,4-tetrahydropyrrolo[1,2-a]pyrazin-7-yl)-4-oxo-4H-chromen-8-yl)ethyl)amino)benzoic acid is prepared in accordance with the method of Example 1.

¹H NMR (400 MHz, DMSO-d6) δ=8.97 (brs, 1H), 7.83 (d, J=7.6 Hz, 1H), 7.73 (s, 1H), 7.53 (s, 1H), 7.14-7.01 (m, 2H), 6.81 (d, J=4.0 Hz, 1H), 6.74 (s, 1H), 6.53-6.46 (m, 1H), 6.36 (br d, J=8.4 Hz, 1H), 5.22-5.11 (m, 1H), 4.72-4.50 (m, 2H), 3.72-3.65 (m, 2H), 2.98 (s, 3H), 2.33 (s, 3H), 1.59 (d, J=6.4 Hz, 3H).

MS m/z (ESI): 486 [M+H]⁺.

### Example 73

### 2-((1-(3,6-dimethyl-2-(5-methyl-4-oxo-4,5,6,7-tetrahydropyrazolo[1,5-a]pyrazin-2-yl)-4-oxo-4H-chromen-8-yl)ethyl)amino)benzoic acid

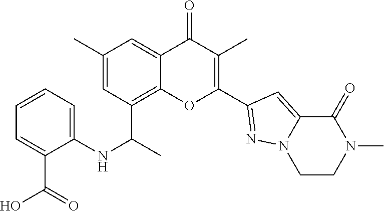 [SMILES: Cc1cc(C(C)Nc2ccccc2C(=O)O)c2oc(-c3cc4n(n3)CCN(C)C4=O)c(C)c(=O)c2c1]

2-((1-(3,6-dimethyl-2-(5-methyl-4-oxo-4,5,6,7-tetrahydropyrazolo[1,5-a]pyrazin-2-yl)-4-oxo-4H-chromen-8-yl)ethyl)amino)benzoic acid is prepared in accordance with the method of Example 1.

¹H NMR (400 MHz, DMSO-d₆) δ=7.81 (d, J=7.2 Hz, 1H), 7.73 (s, 1H), 7.54 (d, J=2.0 Hz, 1H), 7.51 (s, 1H), 7.15 (t, J=7.6 Hz, 1H), 6.89 (s, 1H), 6.54-6.45 (m, 2H), 5.37-5.30 (m, 1H), 4.55 (t, J=6.8 Hz, 2H), 3.86 (t, J=6.4 Hz, 2H), 3.04 (s, 3H), 2.35 (s, 3H), 1.63 (d, J=6.8 Hz, 3H).

MS m/z (ESI): 487 [M+H]⁺.

### Example 74

### 2-((1-(6-methyl-2-(6-methyl-7-oxo-6,7-dihydro-5H-pyrrolo[3,4-b]pyridin-3-yl)-4-oxo-4H-chromen-8-yl)ethyl)amino)benzoic acid

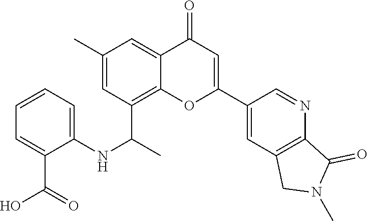 [SMILES: Cc1cc(C(C)Nc2ccccc2C(=O)O)c2oc(-c3cnc4c(c3)CN(C)C4=O)cc(=O)c2c1]

2-((1-(6-methyl-2-(6-methyl-7-oxo-6,7-dihydro-5H-pyrrolo[3,4-b]pyridin-3-yl)-4-oxo-4H-chromen-8-yl)ethyl)amino)benzoic acid is prepared in accordance with the method of Example 1.

¹H NMR (400 MHz, DMSO-d₄) δ ppm 9.43 (d, J=1.63 Hz, 1H) 8.76 (s, 1H) 7.75-7.83 (m, 2H) 7.60 (s, 1H) 7.31 (s, 1H) 7.14-7.24 (m, 1H) 6.47-6.58 (m, 2H) 5.32-5.38 (m, 1H) 4.60 (s, 2H) 3.16 (s, 3H) 2.38 (s, 3H) 1.67 (br d, J=6.50 Hz, 3H) MS m/z (ESI): 470 [M+H]⁺.

### Example 75

### 2-((1-(6-methyl-2-(2-(2-morpholinoethyl)-1-oxoisoindolin-5-yl)-4-oxo-4H-chromen-8-yl)ethyl)amino)benzoic acid

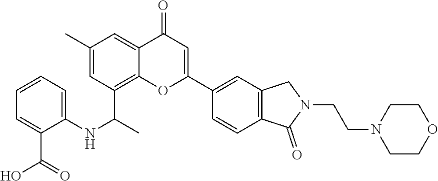 [SMILES: Cc1cc(C(C)Nc2ccccc2C(=O)O)c2oc(-c3ccc4c(c3)CN(CCN3CCOCC3)C4=O)cc(=O)c2c1]

2-((1-(6-methyl-2-(2-(2-morpholinoethyl)-1-oxoisoindolin-5-yl)-4-oxo-4H-chromen-8-yl)ethyl)amino)benzoic acid is prepared in accordance with the method of Example 54.

MS mm/z (ESI): 568 [M+H]⁺.

### Example 76

### 6-chloro-3-((1-(3,6-dimethyl-2-(2-methyl-2H-indazol-5-yl)-4-oxo-4H-chromen-8-yl)ethyl)amino)picolinic acid

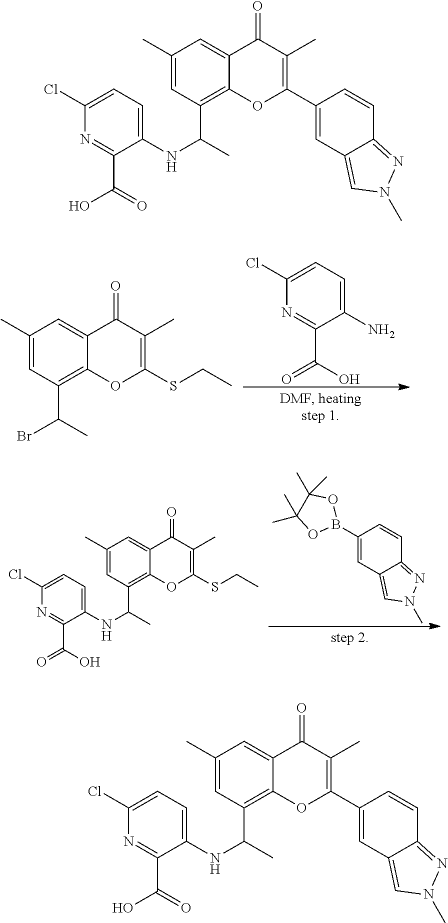

8-(1-bromoethyl)-2-(ethylthio)-3,6-dimethyl-4H-chromen-4-one is prepared in accordance with the method in WO2021202964A1

### Step 1

To mixture of 8-(1-bromoethyl)-2-ethylsulfanyl-3,6-dimethyl-chromen-4-one (387.9 mg, 1.14 mmol) in DMF (8 mL) was added 3-amino-6-chloro-pyridine-2-carboxylic acid (490.38 mg, 2.84 mmol). The mixture was stirred at 80° C. for 16 h. The mixture was filtered and concentrated under reduced pressure to give a crude product. The crude product was purified by prep-HPLC (column: Boston Green ODS 150*30 mm*5 um; mobile phase: [water (FA)-ACN]; B %: 55%-85%, 7 min) to give 6-chloro-3-[1-(2-ethylsulfanyl-3,6-dimethyl-4-oxo-chromen-8-yl)ethylamino]pyridine-2-carboxylic acid (290 mg, 669.87 μmol, 58.9% yield, 100% purity) as yellow solid.

¹H NMR (400 MHz, CDCl₃) δ ppm 10.70 (br s, 1H), 8.27 (d, J=5.2 Hz, 1H), 7.93 (s, 1H), 7.36 (d, J=1.6 Hz, 1H), 7.20 (d, J=9.2 Hz, 1H), 6.75 (d, J=8.8 Hz, 1H), 5.11-5.05 (m, 1 1H), 3.26-3.21 (m, 2H), 2.38 (s, 3H), 2.33 (s, 3H), 1.73 (d, J=6.4 Hz, 3H), 1.51-1.47 (m, 3H).

MS m/z (ESI): 433 [M+H]⁺.

### Step 2

6-chloro-3-((1-(3,6-dimethyl-2-(2-methyl-2H-indazol-5-yl)-4-oxo-4H-chromen-8-yl)ethyl)amino)picolinic acid is prepared in accordance with the method of Example 1.

MS m/z (ESI): 503 [M+H]⁺.

### Example 77

### 6-chloro-3-((1-(3,6-dimethyl-2-(4-morpholinophenyl)-4-oxo-41H-chromen-8-yl)ethyl)amino)picolinic acid

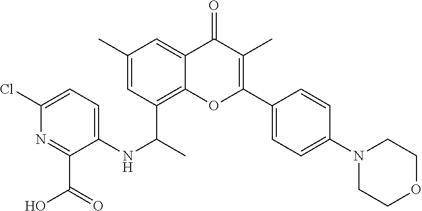 [SMILES: Cc1cc(C(C)Nc2ccc(Cl)nc2C(=O)O)c2oc(-c3ccc(N4CCOCC4)cc3)c(C)c(=O)c2c1]

6-chloro-3-((1-(3,6-dimethyl-2-(4-morpholinophenyl)-4-oxo-4H-chromen-8-yl)ethyl)amino)picolinic acid is prepared in accordance with the method of Example 76.

MS m/z (ESI): 535 [M+H]⁺.

### Example 78

### 2-((1-(3,6-dimethyl-4-oxo-2-(pyrazolo[1,5-a]pyrazin-2-yl)-4H-chromen-8-yl)ethyl)amino)benzoic acid

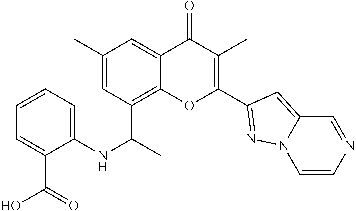 [SMILES: Cc1cc(C(C)Nc2ccccc2C(=O)O)c2oc(-c3cc4cnccn4n3)c(C)c(=O)c2c1]

2-((1-(3,6-dimethyl-4-oxo-2-(pyrazolo[1,5-a]pyrazin-2-yl)-41-H-chromen-8-yl)ethyl)amino)benzoic acid is prepared in accordance with the method of Example 1.

MS m/z (ESI): 455 [M+H]⁺.

### Example 79

### 2-((1-(3,6-dimethyl-2-(2-methylimidazo[1,2-a]pyridin-6-yl)-4-oxo-41H-chromen-8-yl)ethyl)amino)benzoic acid

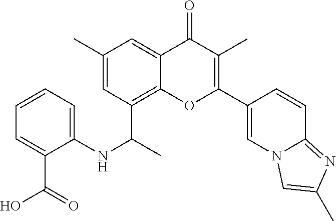 [SMILES: Cc1cc(C(C)Nc2ccccc2C(=O)O)c2oc(-c3ccc4nc(C)cn4c3)c(C)c(=O)c2c1]

2-((1-(3,6-dimethyl-2-(2-methylimidazo[1,2-a]pyridin-6-yl)-4-oxo-4H-chromen-8-yl)ethyl)amino)benzoic acid is prepared in accordance with the method of Example 1.

MS m/z (ESI): 468 [M+H]⁺.

### Example 80

### 2-((1-(3,6-dimethyl-2-(2-methylimidazo[1,2-a]pyrazin-6-yl)-4-oxo-4H-chromen-8-yl)ethyl)amino)benzoic acid

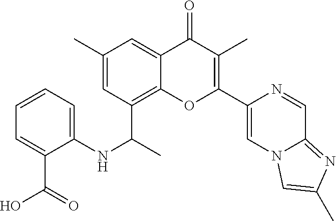 [SMILES: Cc1cc(C(C)Nc2ccccc2C(=O)O)c2oc(-c3cn4cc(C)nc4cn3)c(C)c(=O)c2c1]

2-((1-(3,6-dimethyl-2-(2-methylimidazo[1,2-a]pyrazin-6-yl)-4-oxo-4H-chromen-8-yl)ethyl)amino)benzoic acid is prepared in accordance with the method of Example 1.

MS m/z (ESI): 469 [M+H]⁺.

### Example 81

### 2-((1-(3,6-dimethyl-2-(6-morpholinopyridin-3-yl)-4-oxo-4H-chromen-8-yl)ethyl)amino)benzoic acid

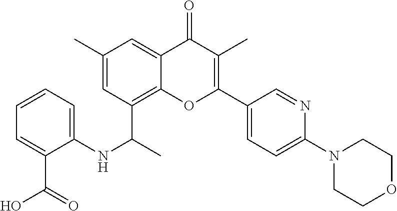 [SMILES: Cc1cc(C(C)Nc2ccccc2C(=O)O)c2oc(-c3ccc(N4CCOCC4)nc3)c(C)c(=O)c2c1]

2-((1-(3,6-dimethyl-2-(6-morpholinopyridin-3-yl)-4-oxo-4H-chromen-8-yl)ethyl)amino)benzoic acid is prepared in accordance with the method of Example 1.

¹H NMR (400 MHz, ACETONITRILE-d₃) δ ppm 12.62-12.96 (m, 1H) 8.60 (d, J=2.38 Hz, 1H) 8.35-8.50 (m, 1H) 7.99 (dd, J=9.01, 2.50 Hz, 1H) 7.81 (dd, J=7.94, 1.44 Hz, 1H) 7.75 (s, 1H) 7.49 (d, J=2.00 Hz, 1H) 7.15-7.24 (m, 1H) 7.00 (d, J=9.01 Hz, 1H) 6.54 (t, J=7.44 Hz, 1H) 6.44 (d, J=8.50 Hz, 1H) 5.08-5.21 (m, 1H) 3.67-3.76 (m, 4H) 3.56-3.64 (m, 4H) 2.31-2.37 (m, 3H) 2.12 (s, 3H) 1.59 (d, J=6.63 Hz, 3H).

MS m/z (ESI): 500 [M+H]⁺.

### Example 82

### 2-((1-(6-methyl-2-(2-methyl-2H-pyrazolo[3,4-b]pyridin-5-yl)-4-oxo-41H-chromen-8-yl)ethyl)amino)benzoic acid

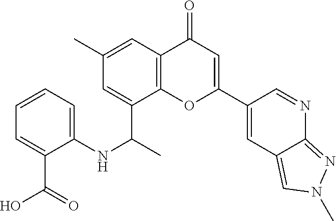 [SMILES: Cc1cc(C(C)Nc2ccccc2C(=O)O)c2oc(-c3cnc4nn(C)cc4c3)cc(=O)c2c1]

2-((1-(6-methyl-2-(2-methyl-2H-pyrazolo[3,4-b]pyridin-5-yl)-4-oxo-4H-chromen-8-yl)ethyl)amino)benzoic acid is prepared in accordance with the method of Example 1.

¹H NMR (400 MHz, DMSO-d₆) δ ppm 8.73 (dd, J=4.13, 1.50 Hz, 1H) 8.44 (dd, J=8.38, 1.50 Hz, 2H) 7.79-7.84 (m, 2H) 7.58 (d, J=1.88 Hz, 1H) 7.31 (dd, J=8.44, 4.19 Hz, 1H) 7.14-7.20 (m, 1H) 6.96 (s, 1H) 6.55 (t, 1=7.44 Hz, 1H) 6.46 (d, J=8.38 Hz, 1H) 5.23 (br t, J=5.94 Hz, 1H) 4.56 (s, 3H) 2.38 (s, 3H) 1.62 (d, J=6.63 Hz, 3H).

MS m/z (ESI): 455 [M+H]⁺.

### Example 83

### 2-((1-(6-methyl-2-(2-methyl-2H-pyrazolo[3,4-c]pyridin-5-yl)-4-oxo-41H-chromen-8-yl)ethyl)amino)benzoic acid

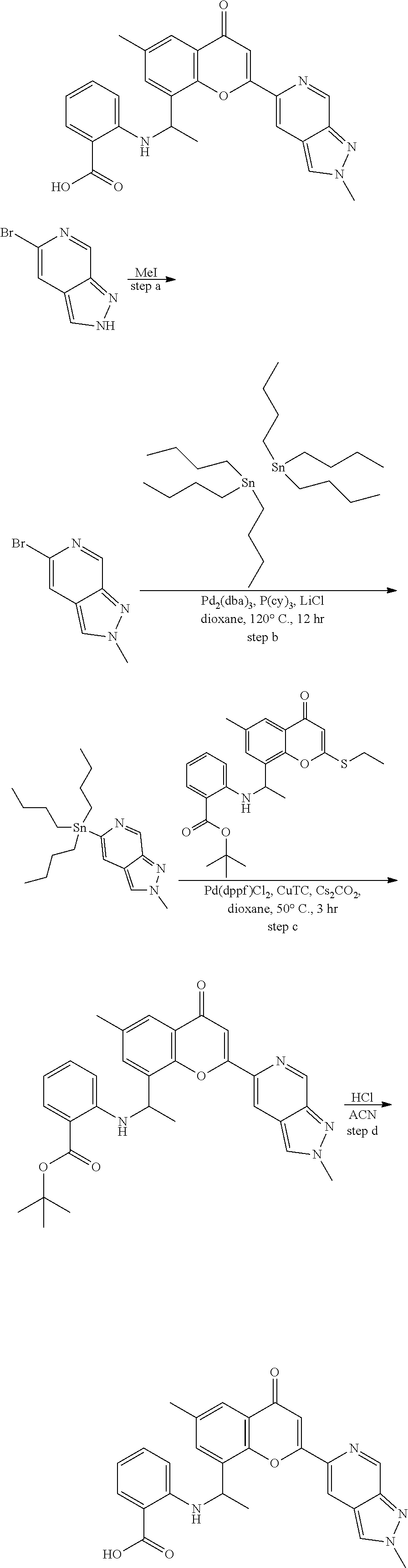

### Step a

5-bromo-2H-pyrazolo[3,4-c]pyridine (2(0) mg, 1.01 mmol, 1 eq) was dissolved in TF (4 mL) followed by addition of NaH (48 mg, 1.21 mmol, 60% purity, 1.2 eq) at 0° C. After the reaction was carried out for 30 min, Met (286 mg, 2.02 mmol, 125.75 uL, 2 eq) was added dropwise. The reaction solution was warmed up to 25′C and stirred 1.5 hr. LCMS showed desired mass was detected. The reaction mixture was added with water (20 ml), extracted with EtOAc (50 ml*3) to give the organic phase. Then dried over Na₂SO₄, filtered and concentrated under reduced pressure to give a residue. The residue was purified by flash silica gel chromatography (ISCO®; 4 g SepaFlash® Silica Flash Column, Fluent of 0-30% Ethyl acetate/Petroleum ether gradient @20 mL/min). Compound 1 5-bromo-2-methyl-pyrazolo[3,4-c]pyridine (120 mg, 565.91 μmol, 56% yield) was obtained as a white solid.

¹H NMR (400 MHz, DMSO-d₆) δ ppm 8.99 (s, 1H) 8.49 (s, 1H) 7.98 (d, J=1.25 Hz, 1H) 4.27 (s, 3H)

MS m/z (ESI): 212 [M+18]⁺.

### Step b

A mixture of 5-bromo-2-methyl-pyrazolo[3,4-c]pyridine (110 mg, 518.75 μmol, 1 eq), tributyl(tributylstannyl)stannane (880 mg, 1.52 mmol, 758.62 uL, 2.92 eq), Pd₂(dba)₃ (47 mg, 51.88 μmol, 0.1 eq), P(Cy)₃ (29 mg, 103.75 μmol, 33.64 uL, 0.2 eq) and LiCl (109 mg, 2.59 mmol, 53.12 uL, 5 eq) in dioxane (5 mL) was degassed and purged with N₂ for 3 times, and then the mixture was stirred at 120° C. for 12 hr under N₂ atmosphere. LCMS showed desired mass was detected. The reaction mixture was filtered to remove the insoluble and concentrated under vacuum to give the crude product. The residue was purified by flash silica gel chromatography (ISCO®; 4 g SepaFlash® Silica Flash Column, Eluent of 30% Ethyl acetate/Petroleum ether gradient @ 25 mL/min). Compound tributyl-(2-methylpyrazolo[3,4-c]pyridin-5-yl)stannane (150 mg, 355.29 μmol, 68% yield) was obtained as a white solid.

MS m/z (ESI): 424[M+18]⁺.

### Step c

A mixture of tert-butyl 2-[1-(2-ethylsulfanyl-6-methyl-4-oxo-chromen-8-yl)ethylamino]benzoate (40 mg, 91.00 μmol, 1 eq), tributyl-(2-methylpyrazolo[3,4-c]pyridin-5-yl)stannane (76 mg, 182.00 μmol, 2 eq), Cs₂CO₃ (59 mg, 182.00 μmol, 2 eq), Pd(dppf)Cl₂·CH₂Cl₂ (14 mg, 18.20 μmol, 0.2 eq) and thiophene-2-carbonyloxycopper (34 mg, 182.00 μmol, 2 eq) in dioxane (2 mL) was degassed and purged with N₂ for 3 times, and then the mixture was stirred at 50° C. for 24 hr under N₂ atmosphere. LCMS showed desired mass was detected. The reaction mixture was extracted with ethyl acetate (20 mL*3). The combined organic layers were dried over [Na₂SO₄], filtered and concentrated under reduced pressure to give a crude product. The residue was purified by flash silica gel chromatography (ISCO®; 4 g SepaFlash® Silica Flash Column, Eluent of 0-100% Ethyl acetate/Petroleum ether gradient @ 30 mL/min). Compound tert-butyl 2-[1-[6-methyl-2-(2-methylpyrazolo[3,4-c]pyridine-5-yl)-4-oxo-chromen-8-yl] ethylamino] benzoate (20 mg, 34.47 μmol, 37% yield, 88% purity) was obtained as a white solid.

MS m/z (ESI): 511 [M+H]⁺.

### Step d

To a solution of tert-butyl 2-[1-[6-methyl-2-(2-methylpyrazolo[3,4-c]pyridin-5-yl)-4-oxo-chromen-8-yl]ethylamino]benzoate (20 mg, 39.17 μmol, 1 eq) in ACN (2 mL) was added HCl (12 M, 13.06 uL, 4 eq). The mixture was stirred at 80° C. for 1 br. LCMS showed desired mass was detected. The reaction mixture was concentrated under vacuum to give the residue. The residue was purified by prep-HPLC (column: Xtimate C18 100*30 mm*10 um; mobile phase: [water (FA)-ACN]; B %: 25%-55%, 10 min). Compound 2-[1-[6-methyl-2-(2-methylpyrazolo [3,4-c]pyridin-5-yl)-4-oxo-chromen-8-yl]ethylamino] benzoic acid (2.3 mg, 5.01 μmol, 12% yield, 99% purity) was obtained as a white solid.

¹H NMR (400 MHz, DMSO-d6) δ ppm 9.30 (s, 1H) 8.67 (s, 1H) 8.63 (s, 1H) 7.83 (br d, J=7.88 Hz, 1H) 7.77 (s, 1H) 7.59 (d, J=1.38 Hz, 1H) 7.21-7.26 (m, 2H) 6.54-6.60 (m, 2H) 5.42-5.47 (m, 1H) 4.32 (s, 3) 2.38 (s, 3H) 1.72 (br d, J=6.50 Hz, 3H) MS m/z (ESI): 455 [M+H]⁺.

### Example 84

### 2-((1-(6-methyl-2-(2-methyl-2H-pyrazolo[4,3-b]pyridin-5-yl)-4-oxo-4H-chromen-8-yl)ethyl)amino)benzoic acid

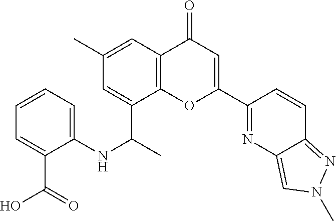 [SMILES: Cc1cc(C(C)Nc2ccccc2C(=O)O)c2oc(-c3ccc4nn(C)cc4n3)cc(=O)c2c1]

2-((1-(6-methyl-2-(2-methyl-2H-pyrazolo[4,3-b]pyri din-5-yl)-4-oxo-4H-chromen-8-yl)ethyl)amino)benzoic acid is prepared in accordance with the method of Example 83.

¹H NMR (400 MHz, ACETONITRILE-d₃) δ ppm 8.91 (s, 1H) 8.47 (br d, J=6.38 Hz, 1H) 8.31 (d, J=9.13 Hz, 1H) 8.14 (d, J=9.13 Hz, 1H) 7.76-7.84 (m, 2H) 7.59 (d, J=1.88 Hz, 1H) 7.27 (s, 1H) 7.20-7.25 (m, 1H) 6.52-6.61 (m, 2H) 5.40 (br t, J=6.32 Hz, 1H) 4.29 (s, 3H) 2.38 (s, 3H) 1.72 (d, J=6.63 Hz, 3H).

MS m/z (ESI): 455 [M+H]⁺.

### Example 85

### 2-((1-(2-(7-fluoro-2-methy-2H-indazol-5-yl)-6-methyl-4-oxo-4H-chromen-8-yl)ethyl)amino)benzoic acid

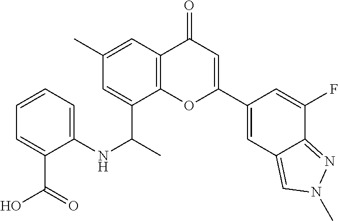 [SMILES: Cc1cc(C(C)Nc2ccccc2C(=O)O)c2oc(-c3cc(F)c4nn(C)cc4c3)cc(=O)c2c1]

2-((1-(2-(7-fluoro-2-methyl-211-indazol-5-yl)-6-methyl-4-oxo-4H-chromen-8-yl)ethyl)amino)benzoic acid is prepared in accordance with the method of Example 1.

¹H NMR (400 MI-z, DMSO-d₆) δ=8.68 (d, J=2.4 Hz, 1H), 8.46 (s, 1H), 7.87-7.70 (m, 3H), 7.57 (s, 1H), 7.25-7.15 (m, 1H), 7.10 (s, 1H), 6.61-6.44 (m, 2H), 5.36 (brs, 1H), 4.24 (s, 3H), 2.36 (s, 3H), 1.69 (br d, J=6.4 Hz, 31H)

MS m/z (ESI): 472 [M+H]⁺.

### Example 86

### 2-((1-(2-(7-chloro-2-methyl-2H-indazol-5-yl)-6-methyl-4-oxo-4H-chromen-8-yl)ethyl)amino)benzoic acid

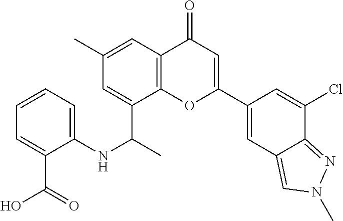 [SMILES: Cc1cc(C(C)Nc2ccccc2C(=O)O)c2oc(-c3cc(Cl)c4nn(C)cc4c3)cc(=O)c2c1]

2-((1-(2-(7-chloro-2-methyl-2H-indazol-5-yl)-6-methyl-4-oxo-4H-chromen-8-yl)ethyl)amino)benzoic acid is prepared in accordance with the method of Example 1.

MS m/z (ESI): 488 [M+H]⁺.

### Example 87

### 2-((1-(2-(7-cyano-2-methyl-2H-indazol-5-yl)-6-methyl-4-oxo-4H-chromen-8-yl)ethyl)amino)benzoic acid

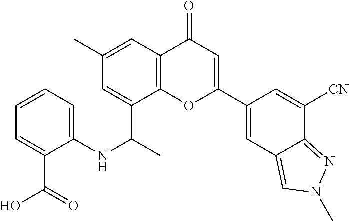 [SMILES: Cc1cc(C(C)Nc2ccccc2C(=O)O)c2oc(-c3cc(C#N)c4nn(C)cc4c3)cc(=O)c2c1]

2-((1-(2-(7-cyano-2-methyl-2H-indazol-5-yl)-6-methyl-4-oxo-4H-chromen-8-yl)ethyl)amino)benzoic acid is prepared in accordance with the method of Example 1.

MS m/z (ESI): 479[M+H]⁺.

### Example 88

### 2-((1-(6-methyl-2-(2-methyl-7-(trifluoromethyl)-2H-indazol-5-yl)-4-oxo-4H-chromen-8-yl)ethyl)amino)benzoic acid

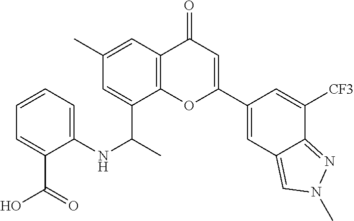 [SMILES: Cc1cc(C(C)Nc2ccccc2C(=O)O)c2oc(-c3cc(C(F)(F)F)c4nn(C)cc4c3)cc(=O)c2c1]

2-((1-(6-methyl-2-(2-methyl-7-(trifluoromethyl)-2H-indazol-5-yl)-4-oxo-4H-chromen-8-yl)ethyl)amino)benzoic acid is prepared in accordance with the method of Example 1.

MS m/z (ESI): 522[M+H]⁺.

### Example 89

### 2-((1-(2-(7-bromo-2-methyl-2H-indazol-5-yl)-6-methyl-4-oxo-4H-chromen-8-yl)ethyl)amino)benzoic acid

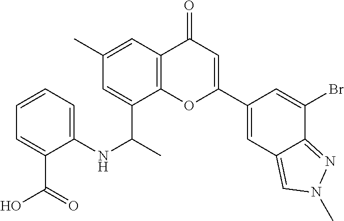 [SMILES: Cc1cc(C(C)Nc2ccccc2C(=O)O)c2oc(-c3cc(Br)c4nn(C)cc4c3)cc(=O)c2c1]

2-((1-(2-(7-bromo-2-methyl-2H-indazol-5-yl)-6-methyl-4-oxo-4H-chromen-8-yl)ethyl)amino)benzoic acid is prepared in accordance with the method of Example 1.

MS m/z (ESI): 532 [M+H]⁺.

### Example 90

### 2-((1-(3,6-dimethyl-2-(4-(4-methylpiperazin-1-yl)phenyl)-4-oxo-4H-chromen-8-yl)ethyl)amino)benzoic acid

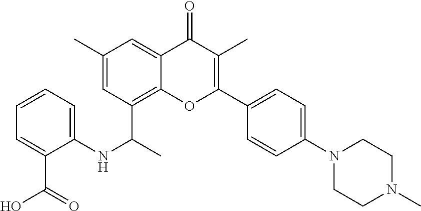 [SMILES: Cc1cc(C(C)Nc2ccccc2C(=O)O)c2oc(-c3ccc(N4CCN(C)CC4)cc3)c(C)c(=O)c2c1]

2-((1-(3,6-dimethyl-2-(4-(4-methylpiperazin-1-yl)phenyl)-4-oxo-4H-chromen-8-yl)ethyl)amino)benzoic acid is prepared in accordance with the method of Example 1.

¹H NMR (400 MHz, ACETONITRILE-d₃) δ ppm 8.51-8.67 (m, 1H) 7.80 (s, 1H) 7.74 (s, 1H) 7.68 (br d, J=8.88 Hz, 2H) 7.49 (s, 1H) 7.26 (br s, 1H) 7.17 (br t, J=7.50 Hz, 1H) 7.09 (br d, J=8.88 Hz, 1H) 6.52 (t, J=7.57 Hz, 1H) 6.41 (br d, J=8.38 Hz, 1H) 5.13 (br s, 1H) 3.17 (br s, 2H) 2.99 (br s, 2H) 2.67 (br s, 2H) 2.44-2.48 (m, 3H) 2.32-2.37 (m, 3H) 2.23 (s, 2H) 2.13 (s, 3H) 1.58 (br d, J=6.63 Hz, 3H).

MS m/z (ESI): 512 [M+H]⁺.

### Example 91

### 2-((1-(3,6-dimethyl-2-(5-morpholinopyridin-2-yl)-4-oxo-4H-chromen-8-yl)ethyl)amino)benzoic acid

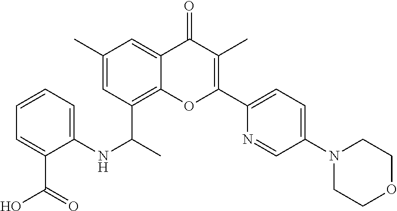 [SMILES: Cc1cc(C(C)Nc2ccccc2C(=O)O)c2oc(-c3ccc(N4CCOCC4)cn3)c(C)c(=O)c2c1]

2-((1-(3,6-dimethyl-2-(5-morpholinopyridin-2-yl)-4-oxo-4H-chromen-8-yl)ethyl)amino)benzoic acid is prepared in accordance with the method of Example 1.

MS m/z (ESI): 500 [M+H]⁺.

### Example 92

### 5-((1-(6-methyl-2-(2-methyl-2H-indazol-5-yl)-4-oxo-4H-chromen-8-yl)ethyl)amino)pyrimidine-4-carboxylic acid

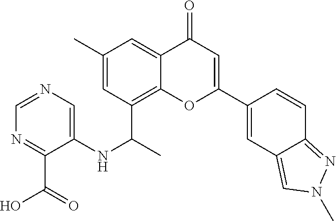 [SMILES: Cc1cc(C(C)Nc2cncnc2C(=O)O)c2oc(-c3ccc4nn(C)cc4c3)cc(=O)c2c1]

5-((1-(6-methyl-2-(2-methyl-2H-indazol-5-yl)-4-oxo-4H-chromen-8-yl)ethyl)amino)pyrimidine-4-carboxylic acid is prepared in accordance with the method of Example 1.

MS m/z (ESI): 456 [M+H]⁺.

### Example 93

### 2-((1-(2-(2-ethyl-2H-indazol-5-yl)-6-methyl-4-oxo-41H-chromen-8-yl)ethyl)amino)benzoic acid

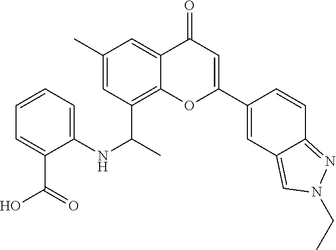 [SMILES: CCn1cc2cc(-c3cc(=O)c4cc(C)cc(C(C)Nc5ccccc5C(=O)O)c4o3)ccc2n1]

2-((1-(2-(2-ethyl-2H-indazol-5-yl)-6-methyl-4-oxo-4H-chromen-8-yl)ethyl)amino)benzoic acid is prepared in accordance with the method of Example 1.

¹H NMR (400 MHz, DMSO-d₆) δ=9.26 (s, 1H), 8.62 (s, 2H), 8.33 (s, 2H), 7.89 (t, J=9.6 Hz, 2H), 7.74 (br d, J=12.8 Hz, 21H), 7.56 (s, 1H), 7.04 (s, 1H), 6.48 (t, J=76 Hz, 1H), 6.40 (d, J 8.4 Hz, 1H), 5.40-5.20 (m, 1H), 4.50 (q, J=7.2 Hz, 2H), 2.35 (s, 3H), 1.66 (d, J=6.4 Hz, 3H), 1.53 (t, J=7. Hz, 3H)

MS m/z (ESI): 468 [M+H]⁺.

### Example 94

### 2-((1-(6-methyl-2-(2-methylisoindolin-5-yl)-4-oxo-4-chromen-8-yl)ethyl)amino)benzoic acid

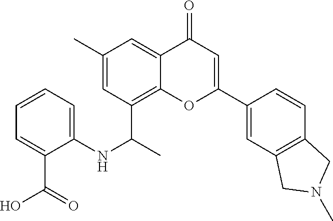 [SMILES: Cc1cc(C(C)Nc2ccccc2C(=O)O)c2oc(-c3ccc4c(c3)CN(C)C4)cc(=O)c2c1]

2-((1-(6-methyl-2-(2-methylisoindolin-5-yl)-4-oxo-4H-chromen-8-yl)ethyl)amino)benzoic acid is prepared in accordance with the method of Example 1.

¹H NMR (400 MHz, DMSO-d6) δ ppm 8.62-8.76 (m, 1H) 8.19-8.21 (m, 1H) 7.91-8.00 (m, 2H) 7.82 (dd, J=7.88, 1.25 Hz, 1H) 7.74 (s, 1H) 7.57 (d, J=1.88 Hz, 1H) 7.43 (d, J=8.50 Hz, 1H) 7.1 (br t, J=7.75 Hz, 1H) 7.02 (s, 1H) 6.44-6.57 (m, 2H) 5.29-5.34 (m, 1H) 3.88 (s, 4H) 3.39 (s, 3H) 2.37 (s, 3H) 1.65 (d, J=6.50 Hz, 3H)

MS m/z (ES): 455 [M+H]⁺.

### Example 95

### 2-((1-(6-methyl-2-(4-(1-methyl-1H-pyrazol-4-yl)phenyl)-4-oxo-4H-chromen-8-yl)ethyl)amino)benzoic acid

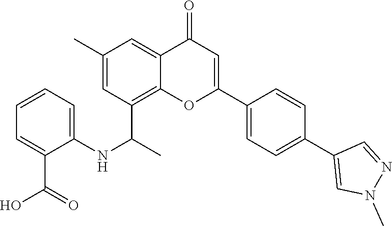 [SMILES: Cc1cc(C(C)Nc2ccccc2C(=O)O)c2oc(-c3ccc(-c4cnn(C)c4)cc3)cc(=O)c2c1]

2-((1-(6-methyl-2-(4-(1-methyl-1H-pyrazol-4-yl)phenyl)-4-oxo-4H-chromen-8-yl)ethyl)amino)benzoic acid is prepared in accordance with the method of Example 1.

¹H NMR (400 MHz, DMSO-d6) δ ppm 8.45 (br d, J=6.25 Hz, 1H) 8.30 (s, 1H) 8.11 (d, J=8.50 Hz, 2H) 8.01 (s, 1H) 7.81 (dd, J=7.94, 1.56 Hz, 1H) 7.73-7.78 (m, 3H) 7.55 (d, J=2.00 Hz, 1H) 7.23 (td, J=7.75, 1.75 Hz, 1H) 7.07 (s, 1H) 6.51-6.58 (m, 2H) 5.34 (br t, J=6.3 Hz, 1H) 3.89 (s, 3H) 2.36 (s, 3H) 1.68 (d, J=6.63 Hz, 3H).

The stereoisomers of example 95: (R)-2-((1-(6-methyl-2-(41-methyl-1H-pyrazol-4-yl)phenyl)-4-oxo-4H-chromen-8-yl)ethyl)amino)benzoic acid and (S)-2-((1-(6-methyl-2-(4-(1-methyl-1H-pyrazol-4-yl)phenyl)-4-oxo-4H-chromen-8-yl)ethyl)amino)benzoic acid

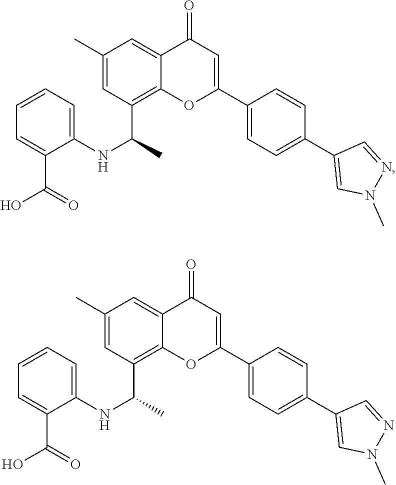 [SMILES: Cc1cc([C@@H](C)Nc2ccccc2C(=O)O)c2oc(-c3ccc(-c4cnn(C)c4)cc3)cc(=O)c2c1 ; Cc1cc([C@H](C)Nc2ccccc2C(=O)O)c2oc(-c3ccc(-c4cnn(C)c4)cc3)cc(=O)c2c1]

The stereoisomers of example 95 were prepared in accordance with the method of Example 20 by chiral SFC (column: ChiralpakIB N-3, 250×30 mm I.D., 5 μm; mobile phase: A for CO₂ and B for ETOH (0.1% NH₃·H₂O); B %: 40%-40%, min; Flow rate: 80 mL/min; Back pressure: 100 bar; Column temperature: 40° C.; Wavelength. 220 nm) to give Peak 1 (Rt=2.07 min) and Peak 2 (Rt=2.27 min).

Peak 1: MS m/z (ESI): 480[M+H]⁺.

¹H NMR (400 MHz, DMSO-d6) δ ppm 8.45 (br d, J=6.25 Hz, 1H) 8.30 (s, 1H) 8.11 (d, J=8.50 Hz, 2H) 8.01 (s, 1H) 7.81 (dd, J=7.94, 1.56 Hz, 1H) 7.73-7.78 (m, 3H) 7.55 (d, J=2.00 Hz, 1H) 7.23 (td, J=7.75, 1.75 Hz, 1H) 7.07 (s, 1H) 6.51-6.58 (m, 2H) 5.34 (br t, J=6.32 Hz, 1H) 3.89 (s, 3H) 2.36 (s, 3H) 1.68 (d, J=6.63 Hz, 3H) Peak 2: MS m/z (ESI): 480[M+H]⁺.

¹H NMR (400 MHz, DMSO-d6) δ ppm 8.45 (br d, J=6.25 Hz, 1H) 8.30 (s, 1H) 8.11 (d, J=8.50 Hz, 2H) 8.01 (s, 1H) 7.81 (dd, J=7.94, 1.56 Hz, 1H) 7.73-7.78 (m, 3H) 7.55 (d, J=2.00 Hz, 1H) 7.23 (td, J=7.75, 1.75 Hz, 1H) 7.07 (s, 1H) 6.51-6.58 (m, 2H) 5.34 (br t, J=6.32 Hz, 1H) 3.89 (s, 3H) 2.36 (s, 3H) 1.68 (d, J=6.63 Hz, 3H)

### Example 96

### 2-((1-(6-methyl-2-(2-(1-methyl-1H-pyrazol-4-yl)pyrimidin-5-yl)-4-oxo-411-chromen-8-yl)ethyl)amino)benzoic acid

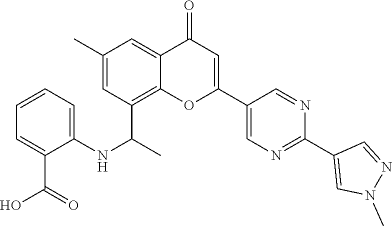 [SMILES: Cc1cc(C(C)Nc2ccccc2C(=O)O)c2oc(-c3cnc(-c4cnn(C)c4)nc3)cc(=O)c2c1]

2-((1-(6-methyl-2-(2-(1-methyl-1H-pyrazol-4-yl)pyrimidin-5-yl)-4-oxo-4H-chromen-8-yl)ethyl)amino)benzoic acid is prepared in accordance with the method of Example 1.

MS m/z (ESI): 482 [M+H]⁺.

### Example 97

### 2-((1-(6-methyl-2-(4-(1-(methylsulfonyl)azetidin-3-yl)phenyl)-4-oxo-4H-chromen-8-yl)ethyl)amino)benzoic acid

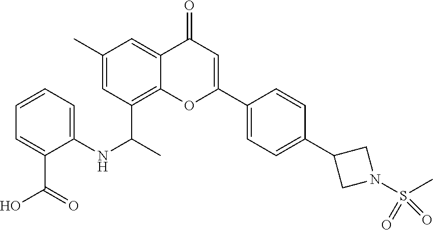 [SMILES: Cc1cc(C(C)Nc2ccccc2C(=O)O)c2oc(-c3ccc(C4CN(S(C)(=O)=O)C4)cc3)cc(=O)c2c1]

2-(1-(6-methyl-2-(4-(1-(methylsulfonyl)azetidin-3-yl)phenyl)-4-oxo-4H-chromen-8-yl)ethyl)amino)benzoic acid is prepared in accordance with the method of Example 1.

MS m/z (ESI): 533 [M+H]⁺.

### Example 98

### 2-((1-(6-chloro-2-(2-methyl-2H-indazol-5-yl)-4-oxo-4H-chromen-8-yl)ethyl)amino)benzoic

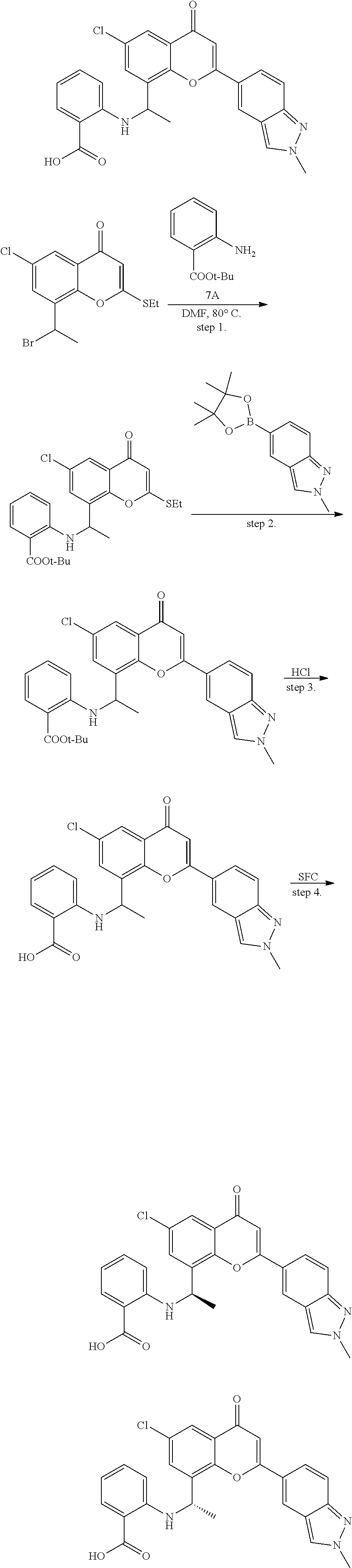

8-(1-bromoethyl)-6-chloro-2-(ethylthio)-4H-chromen-4-one is prepared in accordance with the method in WO2021202964A1

### Step 1

To a solution of 8-(1-bromoethyl)-6-chloro-2-ethylsulfanyl-chromen-4-one (180 mg, 517.76 μmol, 1 eq) in DMF (4 mL) was added tert-butyl 2-aminobenzoate (100 mg, 517.76 μmol, 94.39 uL, 1 eq) at 25° C. The mixture was stirred at 80° C. for 16 h. LCMS showed desired mass was detected. The reaction mixture was quenched with H₂O (6 mL) and extracted with DCM (6 mL*3). The combined organic layers were dried over Na₂SO₄, filtered and concentrated under reduced pressure to give a residue. The residue was purified by flash silica gel chromatography (ISCO®; 4 g SepaFlash® Silica Flash Column, Eluent of 0-30% Ethyl acetate/Petroleum ether gradient @ 18 mL/Min). Compound tert-butyl 2-[1-(6-chloro-2-ethylsulfanyl-4-oxo-chromen-8-yl)ethylamino]benzoate (180 mg, 391.32 μmol, 75.58% yield) was obtained as a yellow solid.

MS m/z (ESI): 460[M+H]⁺.

### Step 2

A mixture of tert-butyl 2-[1-(6-chloro-2-ethylsulfanyl-4-oxo-chromen-8-yl) ethylamino]benzoate (170 mg, 369.58 μmol, 1 eq), 2-methyl-5-(4,4,5,5-tetramethyl-1,3,2-dioxaborolan-2-yl)indazole (143.1 mg, 554.37 μmol, 1.5 eq), Cs₂CO₃ (240.8 mg, 739.1 μmol, 2 eq), thiophene-2-carbonyloxycopper (140.9 mg, 739.16 μmol, 2 eq) and Pd(dppf)Cl₂·CH₂Cl₂ (60.3 mg, 73.92 μmol, 0.2 eq) in dioxane (5 mL) was stirred at 100° C. for 16 h. LCMS showed desired mass was detected. The mixture solution was filtered. The filtrate was evaporated under vacuum to give a residue. The residue was purified by flash silica gel chromatography (ISCO®; 12 g SepaFlIash® Silica Flash Colum n, Eluent of 0˜100% Ethyl acetate/Petroleum ether gradient @ 18 mL/min). Compound tert-butyl 2-[1-[6-chloro-2-(2-methylindazol-5-yl)-4-oxo-chromen-8-yl] ethylamino]benzoate (60 mg, 105.17 μmol, 28.46% yield) was obtained as a yellow solid.

MS m/z (ESI): 530 [M+H]⁺.

### Step 3

HCl (12 M, 14.61 uL, 2 eq) was added to a mixture of tert-butyl 2-[1-[6-chloro-2-(2-methylindazol-5-yl)-4-oxo-chromen-8-yl]ethylamino]benzoate (50 mg, 87.64 μmol, 92.9% purity, 1 eq) in MeCN (3 mL). The mixture was stirred at 80° C. for 1 h. LCMS showed desired mass was detected. The solvent was evaporated under vacuum to give 2-((1-(6-chloro-2-(2-methyl-2H-indazol-5-yl)-4-oxo-41H-chromen-8-yl)ethyl)amino)benzoic acid (40 mg, crude).

The stereoisomers of example 98: (S)-2-((l-(6-chloro-2-(2-methyl-2H-indazol-5-yl)-4-oxo-41-chromen-8-yl)ethyl)amino)benzoic acid and (R)-2-((1-(6-chloro-2-(2-methyl-21H-indazol-5-yl)-4-oxo-4H-chromen-8-yl)ethyl)amino)benzoic acid.

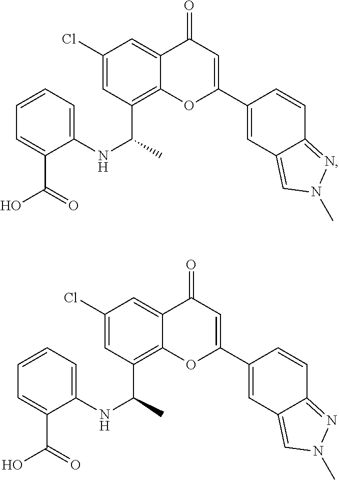 [SMILES: C[C@H](Nc1ccccc1C(=O)O)c1cc(Cl)cc2c(=O)cc(-c3ccc4nn(C)cc4c3)oc12 ; C[C@@H](Nc1ccccc1C(=O)O)c1cc(Cl)cc2c(=O)cc(-c3ccc4nn(C)cc4c3)oc12]

The stereoisomers of example 98 were prepared in accordance with the method of Example 20 by chiral SFC (column: ChiralpakIB N-3, 250×30 mm I.D., 5 pm; mobile phase: A for CO₂ and B for ETOH (0.1% NH₃·H₂O); B %: 50%-50%, min; Flow rate: 80 mL/min; Back pressure: 100 bar; Column temperature: 40° C.; Wavelength: 220 nm) to give Peak 1 (Rt==2.25 min) and Peak 2 (Rt=3.55 min).

Peak 1: MS m/z (ESI): 474 [M+H]⁺.

¹H NMR (400 MHz, DMSO-d₆) δ ppm 8.71 (br s, 1H) 8.65 (s, 1H) 8.59 (s, 1H) 7.93 (dd, J=9.26, 1.50 Hz, 1H) 7.89 (d, J=2.63 Hz, 1H) 7.85 (d, J=7.50 Hz, 1H) 7.75 (d, J=9.13 Hz, 1H)⁷0.68 (d, J=2.50 Hz, 1H) 7.20 (br t, J=7.63 Hz, 1H) 7.14 (s, 1H) 6.57 (t, J=7.57 Hz, 1H) 6.51 (br d, J=8.51 Hz, 1H) 5.39 (br s, 1H) 4.22 (s, 3H) 1.71 (d, J=6.63 Hz, 3H) Peak 2: MS m/z (ESI): 474 [M+H]⁺.

¹H NMR (400 Hz, DMSO-d₆) δ ppm 8.65 (s, 1H) 8.58 (s, 1H) 8.49-8.56 (i, 1H) 793 (dd, J=9.19, 1.56 Hz, 1H) 7.89 (d, J=2.63 Hz, 1H) 7.84 (d, J=7.88 Hz, 1H) 7.75 (d, J=9.13 Hz, 1H) 7.69 (d, J=2.63 Hz, 1H) 7.24 (t, J=7.19 Hz, 1H) 7.14 (s, 1H) 6.49-6.64 (m, 2H) 5.35-5.45 (m, 1H) 4.23 (s, 3H) 1.72 (d, J=6.63 Hz, 3H)

### Example 99

### 2-((1-(6-ethyl-2-(2-methyl-2H-indazol-5-yl)-4-oxo-4H-chromen-8-yl)ethyl)amino)benzoic acid

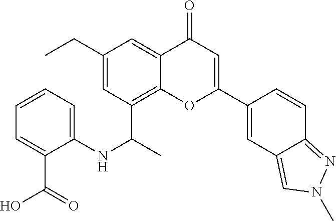 [SMILES: CCc1cc(C(C)Nc2ccccc2C(=O)O)c2oc(-c3ccc4nn(C)cc4c3)cc(=O)c2c1]

2-((1-(6-ethyl-2-(2-methyl-2H-indazol-5-yl)-4-oxo-4H-chromen-8-yl)ethyl)amino)benzoic acid is prepared in accordance with the method of Example 98.

MS m/z (ESI): 468[M+H]⁺.

### Example 100

### 2-((1-(6-cyclopropyl-2-(2-methyl-2H-indazol-5-yl)-4-oxo-4H-chromen-8-yl)ethyl)amino)benzoic acid

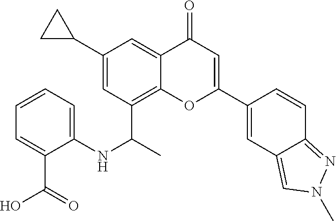 [SMILES: CC(Nc1ccccc1C(=O)O)c1cc(C2CC2)cc2c(=O)cc(-c3ccc4nn(C)cc4c3)oc12]

2-((1-(&-cyclopropyl-2-(2-methyl-2H-indazol-5-yl)-4-oxo-41H-chromen-8-yl)ethyl)amino)benzoic acid is prepared in accordance with the method of Example 98. MS m/z (ES): 480 [M+H]⁺.

### Example 101

### 2-((1-(6-fluoro-2-(2-methyl-2H-indazol-5-yl)-4-oxo-4H-chromen-8-yl)ethyl)amino)benzoic acid

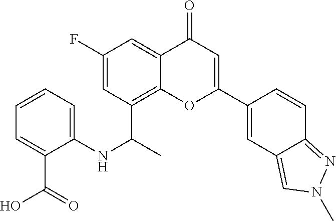 [SMILES: CC(Nc1ccccc1C(=O)O)c1cc(F)cc2c(=O)cc(-c3ccc4nn(C)cc4c3)oc12]

2-(1-(6-fluoro-2-(2-methyl-2H-indazol-5-yl)-4-oxo-4H-chromen-8-yl)ethyl)amino)benzoic acid is prepared in accordance with the method of Example 98.

MS mm/z (ESI): 472 [M+H]⁺.

### Example 102

### 2-((1-(6-methyl-3-(methyl-d3)-2-(2-methyl-2H-indazol-5-yl)-4-oxo-4H-chromen-8-yl)ethyl)amino)benzoic acid

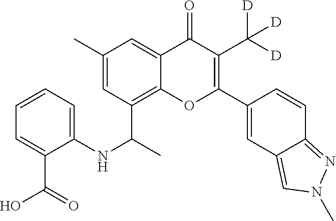 [SMILES: [2H]C([2H])([2H])c1c(-c2ccc3nn(C)cc3c2)oc2c(C(C)Nc3ccccc3C(=O)O)cc(C)cc2c1=O]

2-((1-(6-methyl-3-(methyl-d3)-2-(2-methyl-2H-indazol-5-yl)-4-oxo-4H-chromen-8-yl)ethyl)amino)benzoic acid is prepared in accordance with the method of Example 98.

MS mm/z (ESI): 471 [M+H]⁺.

### Example 103

### 2-((1-(6-bromo-2-(2-methyl-2H-indazol-5-yl)-4-oxo-4H-chromen-8-yl)ethyl)amino)benzoic acid

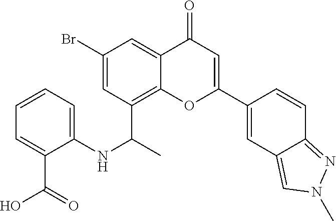 [SMILES: CC(Nc1ccccc1C(=O)O)c1cc(Br)cc2c(=O)cc(-c3ccc4nn(C)cc4c3)oc12]

2-((1-(6-bromo-2-(2-methyl-2H-indazol-5-yl)-4-oxo-41H-chromen-8-yl)ethyl)amino)benzoic acid is prepared in accordance with the method of Example 98.

MS m/z (ESI): 519 [M+H]

### Example 104

### 2-((1-(6-cyano-2-(2-methyl-2H-indazol-5-yl)-4-oxo-4H-chromen-8-yl)ethyl)amino)benzoic acid

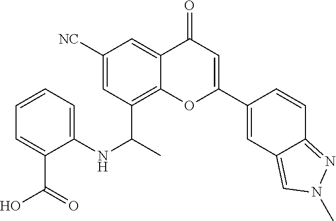 [SMILES: CC(Nc1ccccc1C(=O)O)c1cc(C#N)cc2c(=O)cc(-c3ccc4nn(C)cc4c3)oc12]

2-((1-(6-cyano-2-(2-methyl-2H-indazol-5-yl)-4-oxo-4H-chromen-8-yl)ethyl)amino)benzoic acid is prepared in accordance with the method of Example 98.

MS m/z (ESI): 465[M+1-H]

### Example 105

2-((1-(2-(2-methyl-2H-indazol-5-yl)-4-oxo-6-(trifluoromethyl)-4H-chromen-8-yl)ethyl)amino)benzoic acid

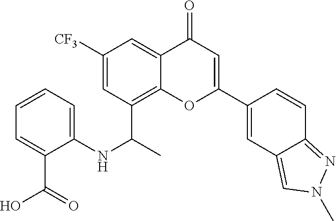 [SMILES: CC(Nc1ccccc1C(=O)O)c1cc(C(F)(F)F)cc2c(=O)cc(-c3ccc4nn(C)cc4c3)oc12]

2-((1-(2-(2-methyl-21H-indazol-5-yl)-4-oxo-6-(trifluoromethyl)-4H-chromen-8-yl)ethyl)amino)benzoic acid is prepared in accordance with the method of Example 98.

The stereoisomers of example 105: (S)-2-((1-(2-(2-methyl-2H-indazol-5-yl)-4-oxo-6-(trifluoromethyl)-411-chromen-8-yl)ethyl)amino)benzoic acid (105A) and (R)-2-((1-(2-(2-methyl-2H-indazol-5-yl)-4-oxo-6-(trifluoromethyl)-4H-chromen-8-yl)ethyl)amino)benzoic acid (105B).

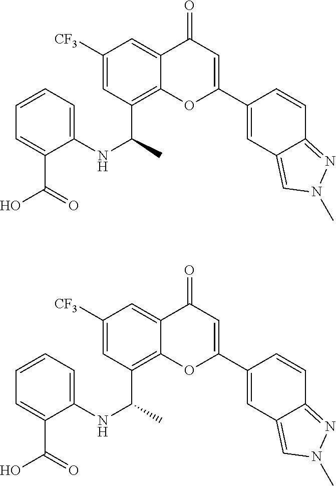 [SMILES: C[C@@H](Nc1ccccc1C(=O)O)c1cc(C(F)(F)F)cc2c(=O)cc(-c3ccc4nn(C)cc4c3)oc12 ; C[C@H](Nc1ccccc1C(=O)O)c1cc(C(F)(F)F)cc2c(=O)cc(-c3ccc4nn(C)cc4c3)oc12]

The stereoisomers of example 105 were prepared in accordance with the method of Example 20 by chiral SFC (column: ChiralpakIB N-3, 250×30 mm I.D., 5 μm; mobile phase: A for CO2 and B for ETOH (0.1% NH3·H2O); B %: 60%-60%, min; Flow rate: 8 mL/min; Back pressure: 100 bar; Column temperature: 40° C.; Wavelength: 220 nm) to give Peak 1 (Rt=1.67 min, 105A) and Peak 2 (Rt=2.64 min, 105B). 105A and 105B were assigned arbitrarily.

Peak 1: MS m/z (ESI): 508 [M+H]+.

¹H NMR (400 MHz, ACETONITRILE-d₃) δ ppm 8.69 (s, 1H) 8.61 (s, 1H) 8.21 (s, 1H) 7.93-7.99 (m, 2H) 7.84 (br d, J=8.13 Hz, 1H) 7.76 (d, J=9.01 Hz, 1H) 7.12-7.25 (m, 2H) 6.44-6.73 (m, 2H) 5.44 (br s, 1H) 5.32 (t, J=4.57 Hz, 1H) 4.23 (s, 3H) 1.74 (br d, J=6.50 Hz, 3H)

Peak 1: MS m/z (ESI): 508 [M+H]⁺.

¹H NMR (400 MHz, ACETONITRILE-d₃) δ ppm 8.70 (s, 1H) 8.61 (s, 1H) 8.20 (s, 1H) 7.95 (br s, 2H) 7.84 (br d, J=7.75 Hz, 1H) 7.76 (d, J=9.01 Hz, 1H) 7.22 (s, 1H) 7.14 (br s, 1H) 6.43-6.72 (m, 2H) 5.43 (br s, 1H) 5.32 (t, J=4.69 Hz, 1H) 4.23 (s, 3H) 1.73 (br d, J=6.38 Hz, 3H)

### Example 106

### 2-((1-(6-methoxy-2-(2-methyl-2H-indazol-5-yl)-4-oxo-4H-chromen-8-yl)ethyl)amino)benzoic acid

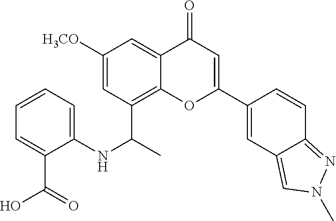 [SMILES: COc1cc(C(C)Nc2ccccc2C(=O)O)c2oc(-c3ccc4nn(C)cc4c3)cc(=O)c2c1]

2-((1-(6-methoxy-2-(2-methyl-2H-indazol-5-yl)-4-oxo-4H-chromen-8-yl)ethyl)amino)benzoic acid is prepared in accordance with the method of Example 98.

MS m/z (ES1): 470 [M+H]⁺.

### Example 107

### 2-((1-(6-(hydroxymethyl)-2-(2-methyl-2H-indazol-5-yl)-4-oxo-4H-chromen-8-yl)ethyl)amino)benzoic acid

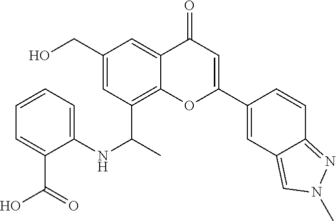 [SMILES: CC(Nc1ccccc1C(=O)O)c1cc(CO)cc2c(=O)cc(-c3ccc4nn(C)cc4c3)oc12]

2-((1-(6-(hydroxymethyl)-2-(2-methyl-2H-indazol-5-yl)-4-oxo-4H-chromen-8-yl)ethyl)amino)benzoic acid is prepared in accordance with the method of Example 98.

MS m/z (ESI): 470 [M+H]⁺.

### Example 108

### 2-((1-(7-methyl-2-(2-methyl-2H-indazol-5-yl)-4-oxo-4H-pyrido[1,2-a]pyrimidin-9-yl)ethyl)amino)benzoic acid

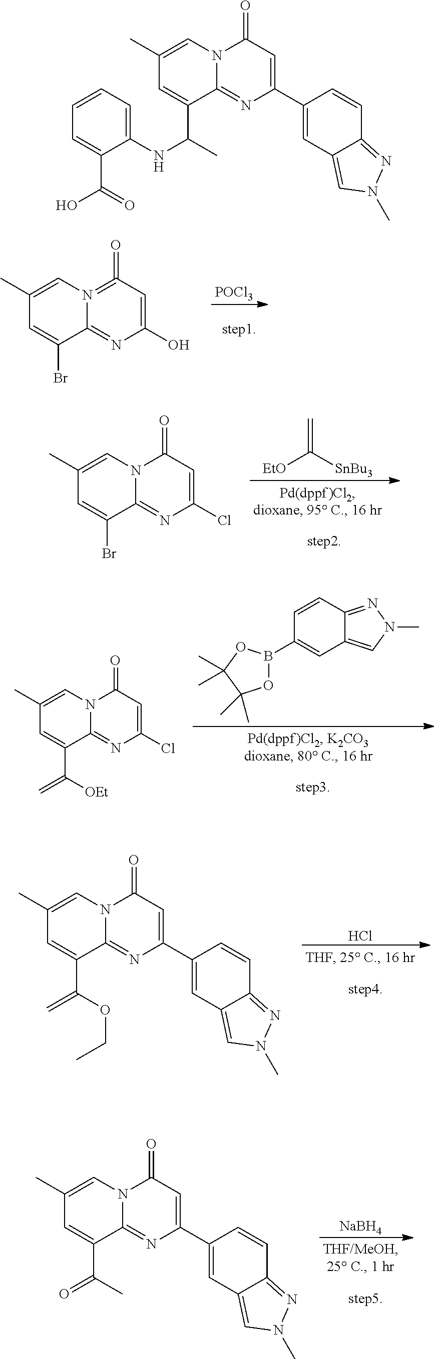

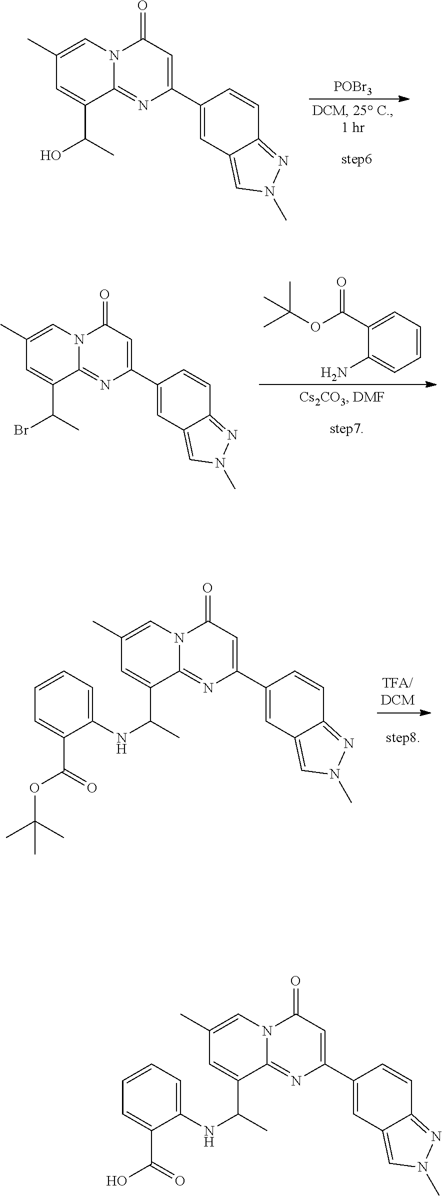

### Step 1

A mixture of 9-bromo-2-hydroxy-7-methyl-pyrido[1,2-a]pyrimidin-4-one (5.8 g, 22.74 mmol, 1 eq) in POCl₃ (23.20 mL) was stirred at 100° C. for 4 hr. The mixture was concentrated under reduced pressure, and quenched with water (200 mL) and extracted with DCM (200 mL*3). The organic layers were combined, washed with brine, dried over Na₂S)₄ and concentrated in vacuo. The residue was purified by flash silica gel chromatography (Eluent of 0˜5% DCM/MeOH) to give 2-chloro-9-(1-ethoxyvinyl)-7-methyl-4H-pyrido[1,2-a]pyrimidin-4-one (727 mg, 1.21 mmol, 33.05% yield, 44% purity) as yellow solid.

LC-MS m/z [M+H]⁺: 275

### Step 2

To a solution of 9-bromo-2-chloro-7-methyl-pyrido[1,2-a]pyrimidin-4-one (1 g, 3.66 mmol, 1 eq) in dioxane (10 mL) was added tributyl(1-ethoxyvinyl)stannane (1.21 g, 3.35 mmol, 1.13 mL, 0.91 eq) and Pd(PPh₃)₂Cl₂ (128.31 mg, 182.81 μmol, 0.05 eq) and purged with N₂ for 3 times, and then the mixture was stirred at 95° C. for 16 hr under N₂ atmosphere. The reaction mixture was quenched by addition EtOAc/KF (1 M)=1:1 (10 mL) at 25° C., filtered and the filtrate was concentrated to dryness to give a residue. The residue was purified by flash silica gel chromatography (Eluent of 0˜60% Ethyl acetate/Petroleum ether) to give 2-chloro-9-(1-ethoxyvinyl)-7-methyl-4H-pyrido[1,2-a]pyrimidin-4-one (727 mg, 1.21 mmol, 33.05% yield, 44% purity) as yellow solid.

LC-MS m/z [M+H]⁺: 265

### Step 3

A mixture of 2-chloro-9-(1-ethoxyvinyl)-7-methyl-pyrido[1,2-a]pyrimidin-4-one (380 mg, 1.44 mmol, 1 eq), 2-methyl-5-(4,4,5,5-tetramethyl-1,3,2-dioxaborolan-2-yl)indazole (420 mg, 1.63 mmol, 1.13 eq), Pd(dppf)₂Cl₂ (210.08 mg, 287.11 μmol, 0.2 eq) and K₂CO₃ (600.40 mg, 434 mmol, 3.03 eq) in dioxane (5 mL) and water (1 mL) was degassed and purged with N₂ for 3 times, and then the mixture was stirred at 80° C. for 16 h under N₂ atmosphere. The mixture was concentrated under reduced pressure to give a residue. The residue was purified by column chromatography (SiO₂, Petroleum ether/Ethyl acetate=2:1 to 0:1) to 9-(1-ethoxyvinyl)-7-methyl-2-(2-methyl-2H-indazol-5-yl)-4H-pyrido[1,2-a]pyrimidin-4-one (630 mg, 57% purity) as a black brown solid.

LC-MS m/z [M i+H]⁺: 361

### Step 4

To a mixture of 9-(1-ethoxyvinyl)-7-methyl-2-(2-methyl-2H-indazol-5-yl)-4H-pyrido[1,2-a]pyrimidin-4-one (630 mg. 1.75 mmol, 1 eq) in THF (2 mL) was added HCl (2 mol/L, 2 mL). The mixture was stirred at 25° C. for 16 h. The mixture was diluted with water (10 mL), and extracted with EtOAc (20 mL×3). The combined extract was washed with brine (10 mL), dried over anhydrous Na₂SO₄, filtered, and concentrated under reduced pressure to give a residue. The residue was s purified by column chromatography (SiO₂, Petroleum ether/Ethyl acetate=50:1 to 0:100) to give 9-acetyl-7-methyl-2-(2-methyl-2H-indazol-5-yl)-41H-pyrido[1,2-a]pyrimidin-4-one (260 mg, 782.30 μmol, 44.75% yield) as a yellow solid. (

LC-MS m/z [M+H]⁺: 333

### Step 5

To a solution of 9-acetyl-7-methyl-2-(2-methyl-2H-indazol-5-yl)-4H-pyrido[1,2-a]pyrimidin-4-one (260 mg, 782.30 μmol, 1 eq) in THE (5 mL) and MeOH (5 mL) was added NaBH₄ (50 mg, 1.32 mmol, 1.69 eq) in portions at 0° C. The mixture was stirred at 25° C. for 1 h. The mixture was diluted with water (10 mL), and then extracted with D)CM (10 mL×3). The combined layers were washed with brine (10 mL), dried over anhydrous Na₂SO₄, filtered, concentrated under reduced pressure to give a residue. The residue was purified by flash silica gel chromatography (Eluent of 0˜100% Ethyl acetate/Petroleum ether) to give 9-(1-hydroxyethyl)-7-methyl-2-(2-methyl-2H-indazol-5-yl)-4H-pyrido[1,2-a]pyrimidin-4-one (150 mg, 448.60 μmol, 57.34% yield) as a yellow solid.

### Step 6

To a solution of 9-(1-hydroxyethyl)-7-methyl-2-(2-methylindazol-5-yl)pyrido[1,2-a]pyrimidin-4-one (200 mg, 598.14 μmol, 1 eq) in DCM (2 mL) was added PBr₃ (485.72 mg, 1.79 mmol, 3 eq) dropwise at 0° C. The mixture was stirred at 25° C. for 3 h. The reaction mixture was quenched with water (10 mL) at 0° C., diluted with DCM (10 mL) and extracted with DCM (10 mL*3). The combined organic layers were washed with brine (10 mL), dried over Na₂SO₄, filtered and concentrated under reduced pressure. The residue was purified by prep-TLC (SiO₂, Petroleum ether:Ethyl acetate=0:1) to give 9-(1-bromoethyl)-7-methyl-2-(2-methyl-2H-indazol-5-yl)-4H-pyrido[1,2-a]pyrimidin-4-one (150 ng, 448.60 μmol, 57.34% yield) as a yellow solid.

LC-MS m/z [M+H]⁺: 397/399

### Step 7

To a mixture of 9-(1-bromoethyl)-7-methyl-2-(2-methylindazol-5-yl)pyrido[1,2-a]pyrimidin-4-one (150 ng, 377.58 μmol, 1 eq), tert-butyl 2-aminobenzoate (87.56 mg, 453.09 μmol, 82.60 uL, 1.2 eq) in ACN (2 mL) was added Cs₂CO₃ (369.07 mg, 1.13 mmol, 3 eq). The mixture was degassed and purged with N₂ for 3 times, and then the mixture was stirred at 80° C. for 12 h tinder N2 atmosphere. The mixture was added cool to rt and ice water (20 ml) was added. The reaction mixture was extracted with EtOAc (30 mL*3). The combined organic layers were washed brine (50 mL), dried over Na₂SO₄, filtered and concentrated under reduced pressure to give a residue. The residue was purified by prep-TLC (SiO₂, PE:EA=0:1) to give tert-butyl 2-((1-(7-methyl-2-(2-methyl-2H-indazol-5-yl)-4-oxo-4H-pyrido[1,2-a]pyrimidin-9-yl)ethyl)amino)benzoate (20 mg, 31.40 μmol, 8.32% yield, 63% purity) as a white solid.

LC-MS m/z [M+H]⁺: 510

### Step 8

To a solution of tert-butyl 2-((1-(7-methyl-2-(2-methyl-2H-indazol-5-yl)-4-oxo-4H-pyrido[1,2-a]pyrimidin-9-yl)ethyl)amino)benzoate in DCM (1 mL) was added TFA (1 mL). The mixture was stirred at 25° C. for 15 h. The reaction mixture was concentrated under reduced pressure. The residue was purified by prep-HPLC (column: Phenomenex luna CIS 150*25 mm*10 um; mobile phase: [water (FA)-ACN]; B %: 35%-65%, 8 min) to give 2-((1-(7-methyl-2-(2-methyl-2H-indazol-5-yl)-4-oxo-4H-pyrido[1,2-a]pyrimidin-9-yl)ethyl)amino)benzoic acid (0.7 mg, 1.54 μmol, 89.52% purity) as white solid.

LC-MS m/z [M+H]⁺:456

¹H NMR (400 MHz, DMSO-d₆) δ ppm 8.74 (s, 2H), 8.53 (s, 1H), 8.22-8.24 (m, 1H), 8.16 (s, 1H), 8.13-8.17 (m, 1H), 8.14 (s, 1H), 7.85 (s, 1H), 7.83-7.86 (m, 1H), 7.06 (s, 1H), 6.52 (t, J=8.0 Hz, 1H), 6.34-6.41 (m, 1H), 5.55-5.62 (m, 1H), 4.21 (s, 3H), 2.34 (s, 3H), 1.69 (d, J=6.4 Hz, 3H)

### Example 109

### 2-((1-(2-(2,7-dimethyl-21-indazol-5-yl)-6-methyl-4-oxo-4H-chromen-8-yl)ethyl)amino)benzoic acid

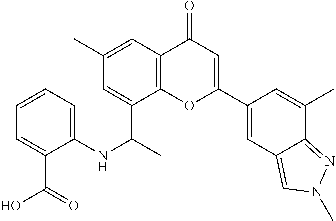 [SMILES: Cc1cc(C(C)Nc2ccccc2C(=O)O)c2oc(-c3cc(C)c4nn(C)cc4c3)cc(=O)c2c1]

2-((1-(2-(2,7-dimethyl-2H-indazol-5-yl)-6-methyl-4-oxo-4H-chromen-8-yl)ethyl)amino)benzoic acid is prepared in accordance with the method of Example 1.

¹H NMR (400 MHz, DMSO-d₆) δ ppm 8.52 (s, 1H) 8.47 (br s, 1H) 8.43 (s, 1H) 7.83 (dd, J=8.13, 1.50 Hz, 1H) 7.75 (d, J=1.25 Hz, 1H) 7.69 (s, 1H) 7.57 (d, J=1.88 Hz, 1H) 7.25 (br t, J=7.88 Hz, 1H) 7.03 (s, 1H) 6.52-6.62 (m, 2H) 5.31-5.44 (m, 1H) 4.22 (s, 3H) 2.57 (s, 3H) 2.37 (s, 3H) 1.70 (d, J=6.63 Hz, 3H) MS m/z (ESI): 468 [M+H]⁺.

### Example 110

### 6-chloro-3-((1-(6-methyl-2-(2-methyl-2-indazol-5-yl)-4-oxo-4H-chromen-8-yl)ethyl)amino)picolinic acid

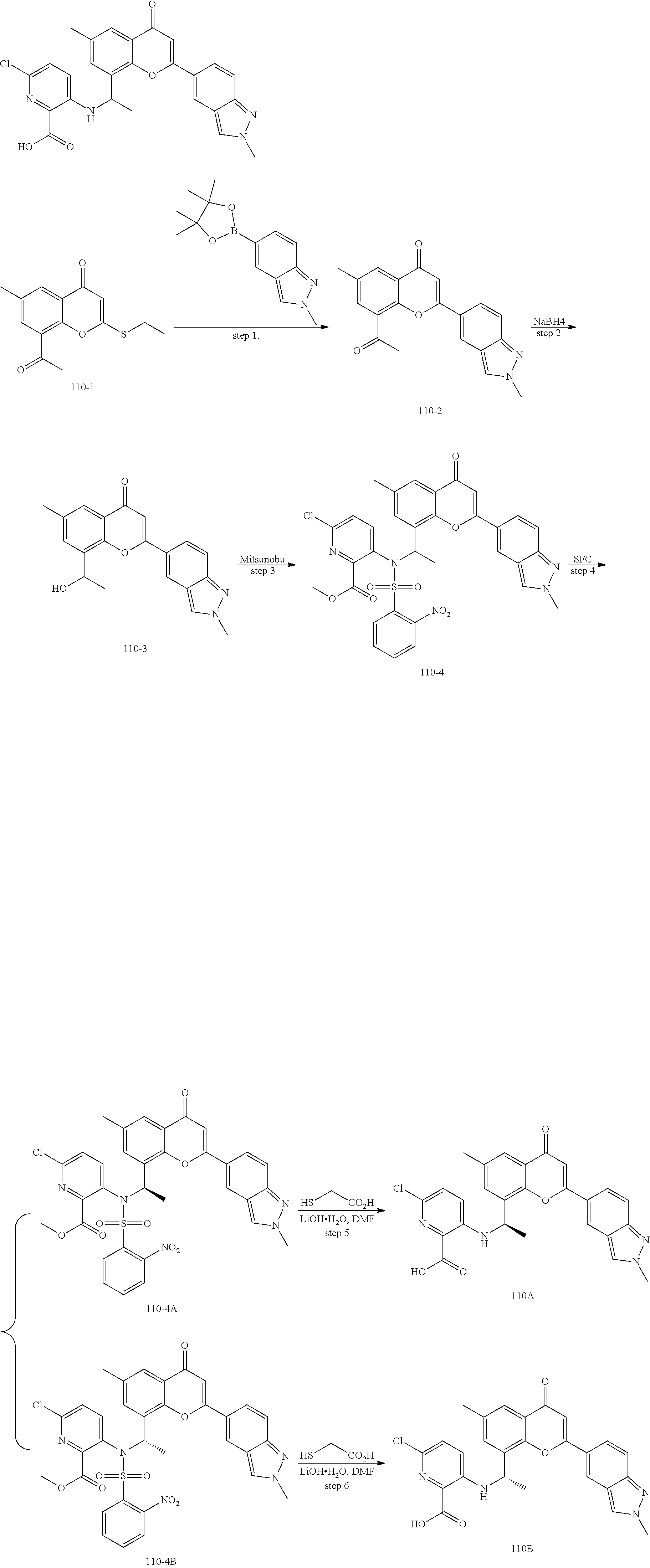

### Step 1

To a solution of 8-acetyl-2-ethylsulfanyl-6-methyl-chromen-4-one (2 g, 7.64 mmol, 1 eq), 2-methyl-5-(4,4,5,5-tetramethyl-1,3,2-dioxaborolan-2-yl)indazole (2.95 g, 11.44 mmol, 1.5 eq), Cs₂CO₃ (4.96 g, 15.24 mmol, 2 eq), thiophene-2-carbonyloxycopper (2.9 g, 15.36 mmol, 2 eq) and Pd(dppf)Cl₂·CH₂Cl₂ (1.24 g, 1.524 mmol, 0.2 eq) in dioxane (20 mL) was degassed and purged with N₂ for 3 times, and then the mixture was stirred at 50° C. for 16 hr under N₂ atmosphere. The reaction mixture was diluted with H₂O (100 mL) and extracted with ethyl acetate (200 mL*3). The combined organic layers were dried over anhydrous Na₂SO₄, filtered and concentrated under reduced pressure to give a crude product. The crude product was purified by flash silica gel chromatography (ISCO®; 20 g SepaFlash® Silica Flash Column, Eluent of 0˜100% Ethyl acetate/Petroleum ether gradient @ 40 mL/min). Compound 8-acetyl-6-methyl-2-(2-methylindazol-5-yl)chromen-4-one (1.16, 3.49 mmol, 45% yield) was obtained as a yellow solid.

MS m/z (ESI): 333 [M+H]⁺.

### Step 2

To a solution of 8-acetyl-6-methyl-2-(2-methylindazol-5-yl)chromen-4-one (1.16 g, 3.49 mmol, 1 eq) in MeOH (20 mL) was added NaBH₄ (0.410 g, 10.84 mmol, 3.1 eq) at 0° C., the mixture was stirred at 20° C. for 2 hr. The reaction mixture was added dropwise into NH₄Cl solution (aq, 100 mL) at 0° C. and extracted with ethyl acetate (200 mL*3). The combined organic layers were dried over [Na₂SO₄], filtered and concentrated under reduced pressure to give a crude product. Compound 8-(1-hydroxyethyl)-6-methyl-2-(2-methylindazol-5-yl)chromen-4-one (910 mg, crude) was obtained as a yellow solid.

MS m/z (ESI): 335 [M+H]⁺.

### Step 3

To a solution of 8-(1-hydroxyethyl)-6-methyl-2-(2-methylindazol-5-yl)chromen-4-one (250 mg, 747.68 μmol, 1 eq), methyl 6-chloro-3-[(2-nitrophenyl)sulfonylamino] pyridine-2-carboxylate (334 mg, 897.22 μmol, 1.2 eq) and PPh₃ (588 mg, 2.24 mmol, 3 eq) in DCM (9 mL) and THF (3 mL). the mixture was stirred at 25° C. for 0.5 hr. Then DIAD (436 uL, 2.24 mmol, 3 eq) was added to the mixture at 0° C., the mixture was degassed and purged with N₂ for 3 times, and then the mixture was stirred at 25° C. for 16 hr under N₂ atmosphere. The reaction mixture was filtered to remove the insoluble and concentrated under vacuum to give the crude product. The crude product was purified by prep-H-PLC (Basic condition: column: Phenomenex C18 75*30 mm*3 um; mobile phase: [water (N-41CO₃)-ACN]; B %: 28%-68/%, 36 min). Compound methyl 6-chloro-3-[1-[6-methyl-2-(2-methylindazol-5-yl)-4-oxo-chromen-8-yl]ethyl-(2-nitrophenyl)sulfonyl-amino]pyridine-2-carboxylate (200 mg, 290.65 μmol, 39% yield) was obtained as a white solid.

MS m/z (ESI): 668 [M+H]⁺.

### Step 4

The stereoisomers of Compound 4: methyl (R)-6-chloro-3-((N-(1-(6-methyl-2-(2-methyl-211-indazol-5-yl)-4-oxo-4H-chromen-8-yl)ethyl)-2-nitrophenyl)sulfonamido)picolinate and methyl (S)-6-chloro-3-((N-(1-(6-methyl-2-(2-methyl-2H-indazol-5-yl)-4-oxo-4H-chromen-8-yl)ethyl)-2-nitrophenyl)sulfonamido)picolinate acid.

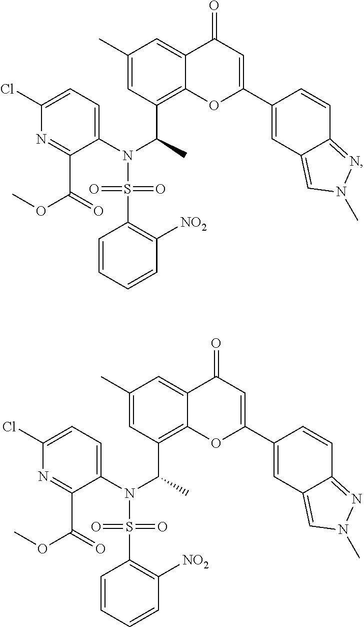 [SMILES: COC(=O)c1nc(Cl)ccc1N([C@H](C)c1cc(C)cc2c(=O)cc(-c3ccc4nn(C)cc4c3)oc12)S(=O)(=O)c1ccccc1[N+](=O)[O-] ; COC(=O)c1nc(Cl)ccc1N([C@@H](C)c1cc(C)cc2c(=O)cc(-c3ccc4nn(C)cc4c3)oc12)S(=O)(=O)c1ccccc1[N+](=O)[O-]]

The stereoisomers of 6-chloro-3-((N-(1-(6-methyl-2-(2-methyl-2H-indazol-5-yl)-4-oxo-4H-chromen-8-yl)ethyl)-2-nitrophenyl)sulfonamido)picolinate acid were prepared in accordance with the method of Example 20 by chiral SFC (column: ChiralpaklB N-3, 250×30 mm I.D., 5 μm; mobile phase: A for CO₂ and B for ETOH (0.1% NH₃·H₂O); B %: 40%-40%, min; Flow rate: 80 mL/min; Back pressure: 100 bar; Column temperature: 40° C.; Wavelength: 220 nm) to give Peak 1 (Rt=2.82 min, 110-4A) and Peak 2 (Rt=4.11 min, 110-4B). 110-4A and 110-4B were assigned arbitrarily.

MS m/z (ESI): 668 [M+H]⁺.

### Step 5

To a solution of 110-4A (75 mg, 108.99 mol, 1 eq) in DMF (0.5 mL) was added LiOH·H₂O (37 mg, 871.96 μmol, 8 eq), then ethanethioic S-acid (31 uL, 435.98 μmol, 4 eq) was added to the mixture under N₂ atmosphere. The mixture was stirred at 80° C. for 3 hr. The reaction mixture was filtered to remove the insoluble to give the crude product. The crude product was purified by prep-HPLC (FA condition, column: Welch Xtimate C18 150*30 mm*5 um; mobile phase: [water (FA)-ACN]; B %: 24%-64%, 25 min). 110A (34.5 mg, 65.98 μmol, 60% yield, 93.5% purity) was obtained as a yellow solid.

¹H NMR (400 MHz, DMSO-d₆) δ ppm 8.60 (d, J=0.88 Hz, 1H) 8.56 (s, 1H) 8.43 (br d, J=6.00 Hz, 1H) 7.90 (dd, J=9.26, 1.75 Hz, 1H) 7.69-7.79 (m, 2H) 7.55 (d, J=2.13 Hz, 1H) 7.36 (d, J=8.88 Hz, 1H) 7.16 (d, J=9.13 Hz, 1H)⁷0.05 (s, 1H) 5.42 (t, J=6.69 Hz, 1H) 4.23 (s, 3H) 2.37 (s, 3H) 1.72 (d, J=6.63 Hz, 3H)

MS m/z (ESI): 489 [M+H]⁺.

### Step 6

To a solution of 110-4B (85 mg, 123.53 μmol, 1 eq) in DMF (0.5 mL) was added LiOH·H₂O (42 mg, 988.22 μmol, 8 eq), then ethanethioic S-acid (35 uL, 494.11 μmol, 4 eq) was added to the mixture under N₂ atmosphere. The mixture was stirred at 80° C. for 3 hr. The reaction mixture was filtered to remove the insoluble to give the crude product. The crude product was purified by prep-HPLC (FA condition, column: Welch Xtimate C18 150*30 mm*5 um; mobile phase: [water (FA)-ACN]; B %: 24%-64%, 25 min). 110B (36.8 mg, 74.24 μmol, 60% yield, 98.6% purity) was obtained as a yellow solid.

¹H NMR (400 MHz, DMSO-d₆) δ ppm 8.61 (s, 1H) 8.56 (s, 1H) 837-8.51 (m, 1H) 7.90 (dd, J=9.19, 1.69 Hz, 1H) 7.70-7.79 (m, 2H) 7.55 (d, J=2.00 Hz, 1H) 7.35 (d, J=8.88 Hz, 1H) 7.16 (d, J=9.13 Hz, 1H) 7.05 (s, 1H) 5.42 (t, J=6.63 Hz, 1H) 4.23 (s, 3H) 2.37 (s, 3H) 1.72 (d, J=6.63 Hz, 3H)

MS m/z (ES): 489 [M+H]⁺.

### Example 111

### 2-((1-(2-(2,3-dimethyl-2H-indazol-5-yl)-6-methyl-4-oxo-4H-chromen-8-yl)ethyl)amino)benzoic acid

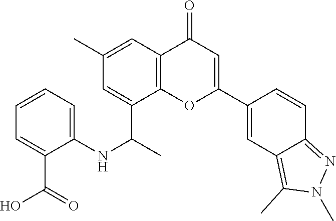 [SMILES: Cc1cc(C(C)Nc2ccccc2C(=O)O)c2oc(-c3ccc4nn(C)c(C)c4c3)cc(=O)c2c1]

2-((1-(2-(2,3-dimethyl-2H-indazol-5-yl)-6-methyl-4-oxo-4H-chromen-8-yl)ethyl)amino)benzoic acid is prepared in accordance with the method of Example 1.

¹H NMR (400 MHz, DMSO-d₆) δ ppm 8.58 (br s, 1H) 7.89 (br d, J=9.01 Hz, 1H) 7.83 (br d, J=7.50 Hz, 1H) 7.75 (br s, 1H) 7.65 (br d, J=8.75 Hz, 114) 7.56 (br s, 1H) 7.18-7.26 (m, 1H) 7.09 (s, 1H) 6.55 (br d, J=6.88 Hz, 2H) 5.39 (br s, 1H) 4.10 (s, 3H) 2.70 (br s, 3H) 2.37 (br s, 3H) 1.72 (br d, J=5.75 Hz, 3H)

MS m/z (ESI): 468 [M+H]⁺.

### Example 112

### 3-((1-(6-methyl-2-(2-methyl-2H-indazol-5-yl)-4-oxo-4H-chromen-8-yl)ethyl)amino)isonicotinic acid

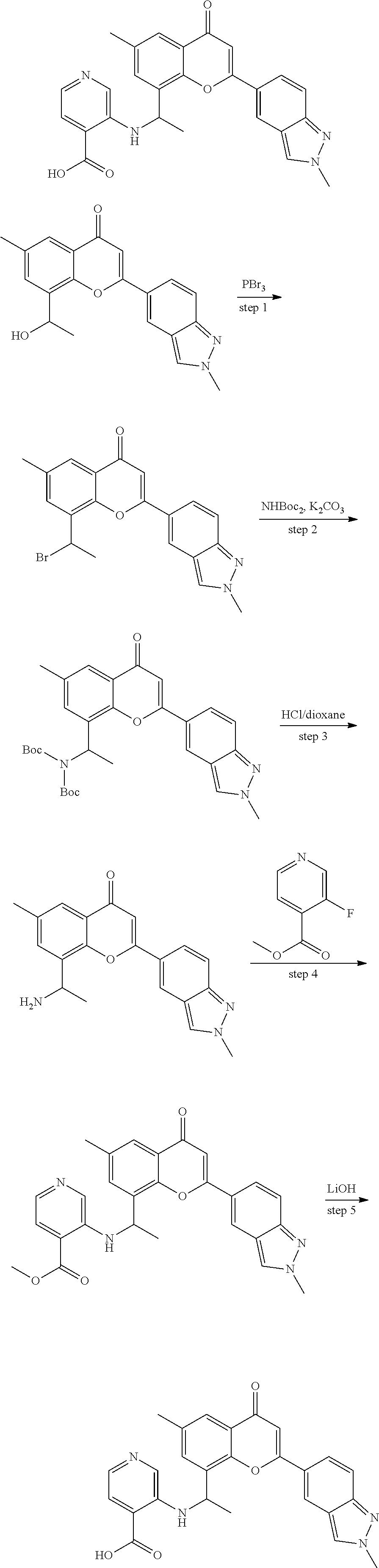

### Step 1

To a solution of 8-(1-hydroxyethyl)-6-methyl-2-methylindazol-5-yl)chromen-4-one (1.85 g, 5.53 mmol, 1 eq) in dichloromethane (30 mL) was added PBr₃ (3.00 g, 11.07 mmol, 2 ml, 2 eq) slowly at 0° C. The mixture was stirred at 0° C. for 2 hr. The reaction mixture was diluted with H₂O (50 mL) at 0° C. and extracted with dichloromethane; (50 mL*3). The combined organic layers were dried over anhydrous Na₂SO₄, filtered and concentrated under reduced pressure to give a crude product. The crude product was purified by flash silica gel chromatography. (ISCO®; 20 g, SepaFlash® R Silica Flash Column, Eluent of 0˜100% N Ethyl acetate/Petroleum ether gradient @ 40 mL/min). Compound 8-(1-bromoethyl)-6-methyl-2-(2-methylindazol-5-yl)chromen-4-one (1.01 g, 2.54 mmol, 46% yield) was obtained as a black solid.

MS m/z (ESI): 399 [M+H]⁺.

### Step 2

To a solution of 8-(1-bromoethyl)-6-methyl-2-(2-methylindazol-5-yl)chromen-4-one (1 g, 2.2 mmol, 1 eq) and tert-butyl N-tert-butoxycarbonylcarbamate (667 mg, 3.07 mmol, 1.22 eq) in DMF (20 mL) was added Cs₂CO₃ (984 mg, 3.02 mmol, 1.2 eq), the mixture was stirred at 50° C. for 10 hr. The reaction mixture was washed with brine and extracted with ethyl acetate (100 mL*3). The combined organic layers were dried over anhydrous Na₂SO₄, filtered and concentrated under reduced pressure to give a crude product. The crude product was purified by flash silica gel chromatography (ISCO®; 12 g SepaFlash® Silica Flash Column, Eluent of 0˜100% Ethyl acetate/Petroleum ether gradient @ 40 mL/min). Compound tert-butyl N-tert-butoxycarbonyl-N-[1-[6-methyl-2-(2-methylindazol-5-yl)-4-oxo-chromen-8-yl]ethyl]carbamate (360 mg, 674.64 μmol, 26% yield) was obtained as a yellow solid.

MS m/z (ESI): 534 [M+H]⁺.

### Step 3

To a solution of tert-butyl N-tert-butoxycarbonyl-N-[1-[6-methyl-2-(2-methylindazol-5-yl)-4-oxo-chromen-8-yl]ethyl]carbamate (360 mg, 674.64 μmol, 1 eq) in dichloromethane (4 mL) was added HCl/dioxane (4 M, 1 mL, 5.93 eq), and the mixture was stirred at 25° C. for 1 hr. The reaction mixture was concentrated under vacuum to give the crude product. Compound 8-(1-aminoethyl)-6-methyl-2-(2-methylindazol-5-yl)chromen-4-one (295 mg, crude, HCl) was obtained as a yellow solid.

MS m/z (ESI): 334 [M+H]⁺.

### Step 4

To a solution of 8-(1-aminoethyl)-6-methyl-2-(2-methylindazol-5-yl)chromen-4-one (80 mg, 239.96 μmol, 1 eq) and methyl 3-fluoropyridine-4-carboxylate (74 mg, 479.93 μmol, 2 eq) in DMF (1.5 mL) was added DIEA (125 uL, 719.89 μmol, 3 eq). The mixture was stirred at 100° C. for 16 hr. The reaction mixture was filtered to remove the insoluble to give the crude product. The crude product was purified by prep-HPLC (FA condition, column: Welch Xtimate C18 150*30 mm*5 um; mobile phase: [water (FA)-ACN]; B %: 20%-60%, 25 min). Compound methyl 3-[1-[6-methyl-2-(2-methylindazol-5-yl)-4-oxo-chromen-8-yl]ethylamino]pyridine-4-carboxylate (30 mg, 64.03 μmol, 26% yield) was obtained as a yellow solid.

MS m/z (ESI): 469 [M+H]⁺.

### Step 5

To a solution of methyl 3-[1-[6-methyl-2-(2-methylindazol-5-yl)-4-oxo-chromen-8-yl]ethyl amino]pyridine-4-carboxylate (27 mg, 57.63 μmol, 1 eq) in THF (0.8 mL) was added LiOH·H₂O (10 mg, 230.52 μmol, 4 eq) in H₂O (0.2 mL). The mixture was stirred at 25° C. for 16 hr. The reaction mixture was filtered to remove the insoluble to give the crude product. The crude product was purified by prep-HPLC (FA condition, column: Welch Xtimate C18 150*30 mm*5 um, mobile phase: [water (FA)-ACN]; 13%: 0%-40%, 25 min). Compound 3-[1-[6-methyl-2-(2-methylindazol-5-yl)-4-oxo-chromen-8-yl]ethylamino]pyridine-4-carboxylic acid (7.3 mg, 15.34 μmol, 26% yield, 95% purity) was obtained as a yellow solid.

¹H NMR (400 MHz, DMSO-d₆) δ ppm 9.28 (br s, 1H) 8.50-8.67 (m, 2H) 8.28 (br s, 2H) 7.91 (br d, J=8.88 Hz, 1H) 7.68-7.80 (m, 4H) 7.52-7.67 (m, 2H) 7.03 (br s, 1H) 5.42 (br s, 1H) 4.21 (br s, 3H) 2.35 (br s, 3H) 1.67 (br s, 3H) MS m/z (ES): 455 [M+H]⁺.

### Example 113

### 2-((1-(6-methyl-2-(2-methyl-2H-indazol-5-yl)-4-oxo-4H-chromen-8-yl)ethyl)amino)nicotinic acid

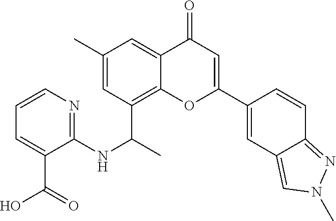 [SMILES: Cc1cc(C(C)Nc2ncccc2C(=O)O)c2oc(-c3ccc4nn(C)cc4c3)cc(=O)c2c1]

2-((1-(6-methyl-2-(2-methyl-2H-indazol-5-yl)-4-oxo-4H-chromen-8-yl)ethyl)amino)nicotinic acid is prepared in accordance with the method of Example 112.

¹H NMR (400 MHz, DMSO-d₆) δ ppm 8.80 (br s, 1H) 8.56 (s, 2H) 8.22 (br d, J=3.13 Hz, 1H) 8.07 (dd, J=7.63, 1.88 Hz, 1H) 7.89 (dd, J=9.26, 1.50 Hz, 1H) 7.69-7.76 (m, 2H) 7.64 (d, J=1.88 Hz, 1H) 7.03 (s, 1H) 6.62 (dd, J=7.63, 4.88 Hz, 1H) 6.05 (br t, J=7.13 Hz, 1H) 4.21 (s, 3H) 2.40 (s, 3H) 1.68 (d, J=6.75 Hz, 3H)

MS m/z (ESI): 455 [M+H]⁺.

### Example 114

### 3-((1-(6-methyl-2-(2-methyl-21-indazol-5-yl)-4-oxo-4H-chromen-8-yl)ethyl)amino)picolinic acid

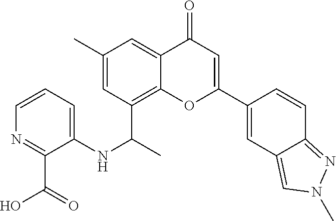 [SMILES: Cc1cc(C(C)Nc2cccnc2C(=O)O)c2oc(-c3ccc4nn(C)cc4c3)cc(=O)c2c1]

3-((1-(6-methyl-2-(2-methyl-2H-indazol-5-yl)-4-oxo-4H-chromen-8-yl)ethyl)amino)picolinic acid is prepared in accordance with the method of Example 112.

¹H NMR (400 MHz, DMSO-d₆) δ ppm 9.72 (br s, 1H) 8.59 (br dd, J=10.07, 8.07 Hz, 2H) 8.35 (br s, 1H) 7.89 (br d, J=6.88 Hz, 1H) 7.72 (br s, 2H)⁷0.51 (br s, 1H) 7.38 (br d, J=1.75 Hz, 1H) 6.99-7.11 (m, 2H) 6.87-6.97 (m, 1H) 5.29 (br s, 1H) 4.22 (br s, 3H) 2.32 (br s, 3H) 1.67 (br d, J=4.13 Hz, 3H) MS m/z (ESI): 455[M+H]⁺.

### Example 115

### (R)-2-((1-(2-(5-(4-acetylpiperazin-1-yl)pyridin-2-yl)-3,6-dimethyl-4-oxo-4H-chromen-8-yl)ethyl)amino)benzoic acid

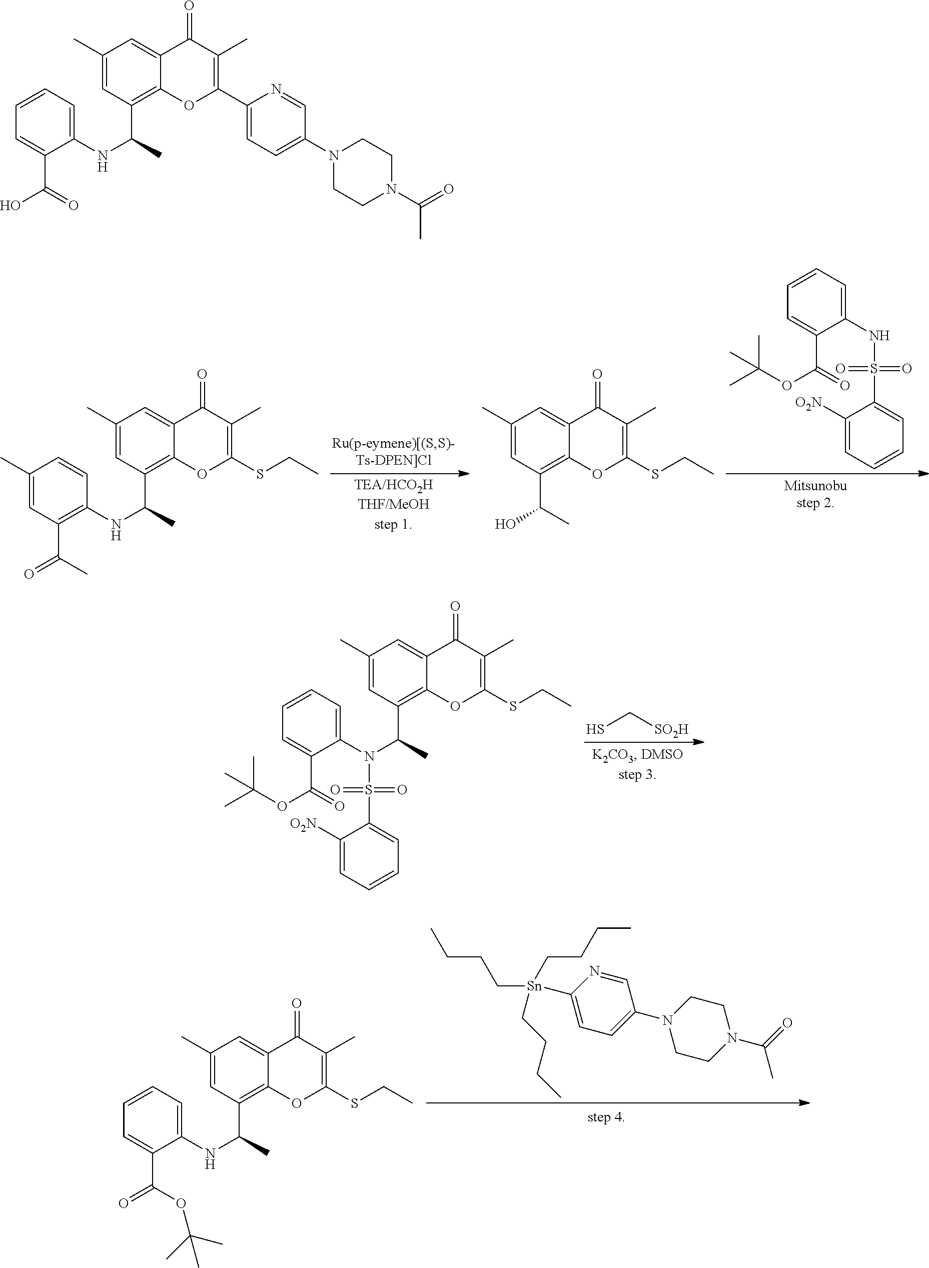

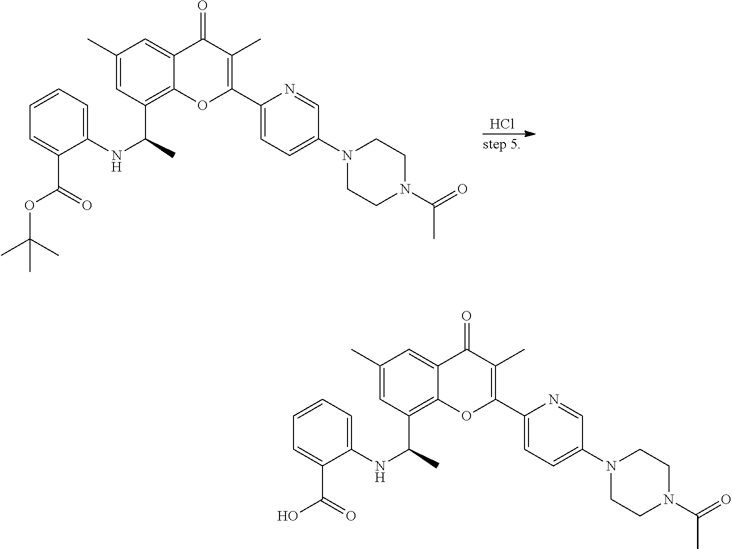 [SMILES: CC(=O)N1CCN(c2ccc(-c3oc4c([C@@H](C)Nc5ccccc5C(=O)OC(C)(C)C)cc(C)cc4c(=O)c3C)nc2)CC1 ; Cl ; CC(=O)N1CCN(c2ccc(-c3oc4c([C@@H](C)Nc5ccccc5C(=O)O)cc(C)cc4c(=O)c3C)nc2)CC1]

### Step 1

To a solution of 8-acetyl-2-ethylsulfanyl-3,6-dimethyl chromen-4-one (3.5 g, 12.67 mmol, 1 eq) and N-[(1 S,2S)-2-amino-1,2-diphenyl-ethyl]-4-methyl-benzenesulfonamide; chlororuthenium; 1-isopropyl-4-methylbenzene (403.5 mg, 633.25 μmol, 0.05 eq) in THF (30 mL) was added a solution of TEA (384 g, 3800 mmol. 5.29 mL, 3 eq) and FORMIC A ((2.91 g. 6333 mmol, 2.39 mL, 5 eq) in THF (30 mL). The mixture was stirred at 60° C. for 4 hr. LCMS showed desired mass was detected. The reaction mixture was concentrated under vacuum to give the crude product. The residue was purified by flash silica gel chromatography (ISCO®; 40 g SepaFlash® Silica Flash Column, Fluent of 0-5% Ethyl acetate/Petroleum ether gradient @ 40 mL/min) to give compound 2-ethylsulfanyl-8[(1 S)-1-hydroxyethyl]-3,6-dimethyl-chromen-4-one (3.4 g, 12.21 mmol, 96% yield) as a yellow solid.

MS m/z (ESI): 279[M+H]⁺.

### Step 2

To a solution of 2-ethylsulfanyl-8-[(1S)-1-hydroxyethyl]-3,6-dimethyl-chromen-4-one (1.8 g, 6.47 mmol. 1 eq) and tort-butyl 2-[(2-nitrophenyl)sulfonylamino]benzoate (2.69 g, 7.11 mmol, 1.1 eq) in Me ((80 mL) was added PPh₃ (3.39 g, 12.93 mmol, 2 eq) and stirred for 0.5 hr. Then DIAD (2.62 g, 12.93 mmol, 2.51 mL, 2 eq) was added at 0° C. The mixture was stirred at 25° C. for 12 hr. LCMS showed desired mass was detected. The reaction mixture was concentrated tinder vacuum to give the crude product. The residue was purified by flash silica gel chromatography (ISCO®; 80 g SepaFlash® Silica Flash Column, Eluent of 0-50% Ethyl acetate/Petroleum ether gradient @ 50 mL/min) to give compound tert-butyl 2-[[(1R)-1-(2-ethylsulfanyl-3,6-dimethyl-4-oxo-chromen-8-yl)ethyl]-(2-nitrophenyl)sulfonyl-amino]benzoate (3 g, 4.70 mmol, 72% yield) was obtained as a white solid.

MS m/z (ESI): 639[M+H]⁺.

### Step 3

To a solution of tert-butyl 2-[[(1R)-1-(2-ethylsulfanyl-3,6-dimethyl-4-oxo-chromen-8-yl) ethyl]-(2-nitrophenyl)sulfonyl-amino]benzoate (1 g, 1.57 mmol, 1 eq) in DMF (10 mL) was added LiOH·H₂O (262.7 mg, 6.26 mmol, 4 eq). Then ethanethioic S-acid (238.3 mg, 3.13 mmol, 22274 μL, 2 eq) was added under N₂ atmosphere. The mixture was stirred at 80° C. for 4 hr. LCMS showed desired mass was detected. The reaction mixture was added water (20 ml) and extracted with ethyl acetate (30 ml*3). The combined organic layers were dried over Na₂SO₄, filtered and concentrated under reduced pressure to give a crude product. The residue was purified by flash silica gel chromatography (ISCO®; 12 g SepaFlash® Silica Flash Column, Eluent of 0-5% Ethyl acetate/Petroleum ether gradient (25 mL/min) to give compound tert-butyl 2-R[[(1R)-1-(2-ethylsulfanyl-3,6-dimethyl-4-oxo-chromen-8-yl)ethyl]amino]benzoate (300 mg. 661.39 μmol, 42% yield) was obtained as a yellow solid.

MS m/z (ESI): 454[M+H]⁺.

### Step 4

To a solution of tert-butyl 2-[[(1R)-1-(2-ethylsulfanyl-3,6-dimethyl-4-oxo-chromen-8-yl) ethyl]amino]benzoate (70 mg, 154.32 μmol, 1 eq) and 1-[4-(6-tributylstannyl-3-pyridyl) piperazin-1-yl]ethanone (152.5 mg, 308.65 μmol, 2 eq) in dioxane (4 mL) was added Cs₂CO₃ (100.5 mg, 308.65 μmol, 2 eq), thiophene-2-carbonyloxycopper (58.8 mg, 308.65 μmol, 2 eq) and Pd(dppf)Cl₂·CH₂Cl₂ (25.2 mg, 30.86 μmol, 0.2 eq). The mixture was stirred at 100° C. for 3 hr under N₂ atmosphere. LC-MS showed tert-butyl 2-[[(1R)-1H-(2-ethylsulfanyl-3,6-dimethyl-4-oxo-chromen-8-yl) ethyl]amino]benzoate was consumed and one main peak with desired mass was detected. The reaction mixture was added water (10 ml) and extracted with ethyl acetate (10 ml*3). The combined organic layers were dried over [Na₂SO₄], filtered and concentrated under reduced pressure to give a crude product. The residue was purified by flash silica gel chromatography (ISCO®; 4 g SepaFlash®) Silica Flash Column, Eluent of 0-100% Ethyl acetate/Petroleum ether gradient @. 20 mL/min). Compound tert-butyl 2-[[(1R)-1-R[2-[5-(4-acetylpiperazin-1-yl)-2-pyridyl]-3,6-dimethyl-4-oxo-chromen-8-yl]ethyl]amino]benzoate (80 mg, 134.07 μmol, 86% yield) was obtained as a black solid.

MS m/z (ESI): 597[M+H]⁺.

### Step 5

To a solution of tert-butyl 2-[[(1R)-1-[2-[5-(4-acetylpiperazin-1-yl)-2-pyridyl]-3,6-dimethyl-4-oxo-chromen-8-yl]ethyl]amino]benzoate (75 mg, 125.69 μmol, 1 eq) in ACN (1 mL) was added HCl (12 M, 20.95 uL, 2 eq). The mixture was stirred at 80° C. for 1 hr. LC-MS showed tert-butyl 2-[[(1R)-1-[2-[5-(4-acetylpiperazin-1-yl)-2-pyridyl]-3,6-dimethyl-4-oxo-chromen-8-yl]ethyl]amino]benzoate was consumed and one main peak with desired mass was detected. The reaction mixture was filtered to give filtrate. The filtrate was purified by prep-HPLC (column: Xtimate C18 100*30 mm*10 um; mobile phase: [water (FA)-ACN]; B %: 50%-80%, 10 min). Compound 2-[[(1R)-1-[2-[5-(4-acetylpiperazin-1-yl)-2-pyridyl]-3,6-dimethyl-4-oxo-chromen-8-yl]ethyl]amino]benzoic acid (12.9 mg, 23.47 μmol, 18% yield, 98% purity) was obtained as a yellow solid.

¹H NMR (400 MHz, ACETONITRILE-d₃) δ ppm 12.80 (br d, J=1.88 Hz, 1H) 8.53 (d, J=2.75 Hz, 1H) 8.37-8.46 (m, 1H) 7.95 (d, J=8.88 Hz, 1H) 7.81 (dd, J=8.00, 1.25 Hz, 1H) 7.75 (s, 1H) 7.46-7.53 (m, 2H) 7.21 (s, 1H) 6.52-6.58 (m, 1H) 6.45-6.49 (m, 1H) 5.21 (br t, J=6.44 Hz, 1H) 3.57-3.63 (m, 4H) 3.41-3.46 (in 2H) 3.36-3.40 (m, 2H) 2.35 (s, 3H) 2.31 (s, 3H) 2.06 (s, 3H) 1.62 (d, J=6.63 Hz, 3H) MS m/z (ESI): 541 [M+H]⁺.

### Example 116

### 2-((1-(6-methyl-4-oxo-2-(1H-pyrrolo[3,2-e]pyridin-2-yl)-4H-chromen-8-yl)ethyl)amino)benzoic acid

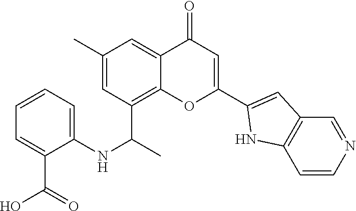 [SMILES: Cc1cc(C(C)Nc2ccccc2C(=O)O)c2oc(-c3cc4cnccc4[nH]3)cc(=O)c2c1]

2-((1-(6-methyl-4-oxo-2-(1H-pyrrolo[3,2-c]pyridin-2-yl)-4H-chromen-8-yl)ethyl)amino)benzoic acid is prepared in accordance with the method of Example 1.

¹H NMR (400 MHz, DMSO-d₆) δ ppm 8.98 (s, 1H) 8.53 (br s, 1H) 8.31 (br d, J=5.63 Hz, 1H) 8.16 (s, 1H) 7.80-7.86 (m, 1H) 7.74 (d, J=1.25 Hz, 1H) 7.55-7.59 (m, 2H) 7.51 (br d, J=5.75 Hz, 1H) 7.20-7.26 (m, 1H) 7.08 (s, 1H) 6.53-6.59 (m, 2H) 5.47 (br d, J=5.50 Hz, 1H) 2.36 (s, 3H) 1.68 (d, J=6.63 Hz, 3H) MS mm/z (ESI): 440 [M+H]⁺.

### Example 117

### 2-((1-(6-methyl-4-oxo-2-(1H-pyrrolo[2,3-c]pyridin-2-yl)-4H-chromen-8-yl)ethyl)amino)benzoic acid

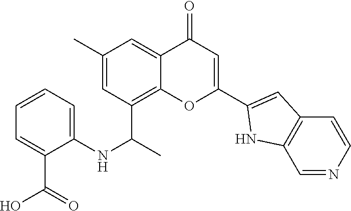 [SMILES: Cc1cc(C(C)Nc2ccccc2C(=O)O)c2oc(-c3cc4ccncc4[nH]3)cc(=O)c2c1]

2-((1-(6-methyl-4-oxo-2-(1-pyrrolo[2,3-c]pyridin-2-yl)-4H-chromen-8-yl)ethyl)amino)benzoic acid is prepared in accordance with the method of Example 1.

¹H NMR (400 MHz, DMSO-d₆) δ ppm 8.93 (s, 1H) 8.45-8.54 (m, 1H) 8.20 (d, J=5.50 Hz, 1H) 7.82 (dd, J=7.88, 1.50 Hz, 1H) 7.75 (d, J=1.25 Hz, 1H) 7.65 (dd, J=5.57, 0.94 Hz, 1H) 7.56 (d, J=2.00 Hz, 1H) 7.47 (s, 1H) 7.19-7.26 (m, 1H) 7.15 (s, 1H) 6.52-6.59 (m, 2H) 5.50 (br t, J==6.38 Hz, 1H) 2.36 (s, 3H) 1.67 (d, J=6.63 Hz, 3H) MS m/z (ESI): 440 [M+H]⁺.

### Example 118

### (R)-2-((1-(3,6-dimethyl-2-(4-(4-(oxetan-3-yl)piperazin-1-yl)phenyl)-4-oxo-4H-chromen-8-yl)ethyl)amino)benzoic acid

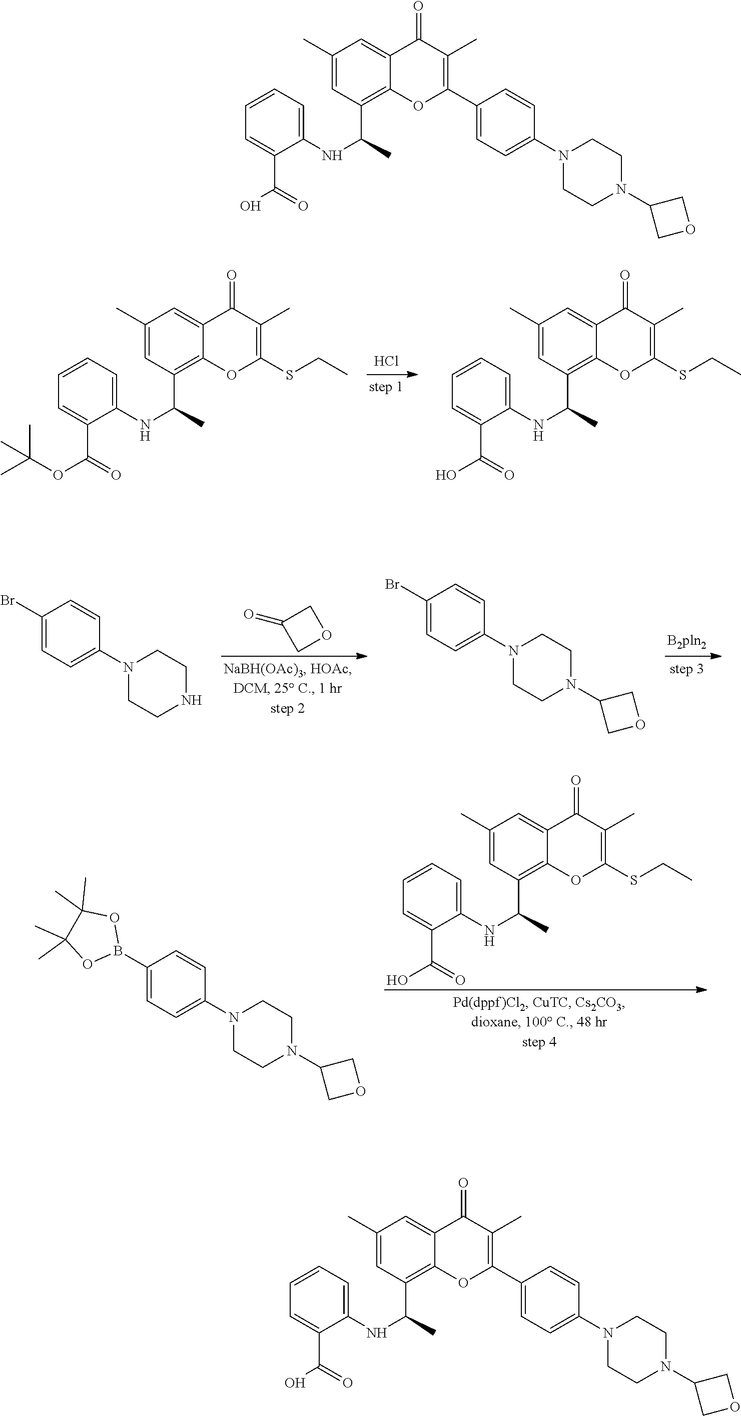

### Step 1

To a solution of tert-butyl 2-[[(1R)-1-(2-ethylsulfanyl-3,6-dimethyl-4-oxo-chromen-8-yl) ethyl]amino] benzoate (70 mg, 154.32 μmol, 1 eq) in ACN (1 mL) was added IC (12 M, 38.5 uL, 3 eq). The mixture was stirred at 80° C. for 1 hr. The reaction mixture was concentrated under reduced pressure to give the crude product. The crude product was without purification. Compound 2-[[(1R)-1-(2-ethylsulfanyl-3,6-dimethyl-4-oxo-chromen-8-yl) ethyl] amino] benzoic acid (60 mg, crude) was obtained as a white solid.

MS mm/z (ESI): 398 [M+H]⁺.

### Step 2

To a solution of 1-(4-bromophenyl)piperazine (1 g, 4.15 mmol, 1 eq), oxetan-3-one (448.2 mg, 6.22 mmol, 1.5 eq) and NaBH(OAc)₃ (1.32 g, 6.22 mmol, 1.5 eq) in CM (1 ml) was added CH₃COOH (249.05 mg, 4.15 mmol, 237.2 uL, 1 eq) at 25° C. The reaction mixture was stirred at 25° C. for 1 hr. The mixture was quenched with sat.NaHCO₃ (20 mL) and extracted with DCM (30 mL,*3). The organic layer dried over Na2SO4, filtered and under vacuum to give crude product. The crude product was without purification. Compound 1-(4-bromophenyl)-4-(oxetan-3-yl)piper azine (1 g, crude) was obtained as a white solid.

MS m/z (ESI): 299 [M+H]⁺.

### Step 3

To a solution of 1-(4-bromophenyl)-4-(oxetan-3-yl)piperazine (800 mg, 2.69 mmol, 1 eq), 4,4,5,5-tetramethyl-2-(4,4,5,5-tetramethyl-1,3,2-dioxaborolan-2-yl)-1,3,2-dioxaborolane (1.37 g, 5.38 mmol, 2 eq) and KOAc (792.56 mg, 8.08 mmol, 3 eq) in dioxane (15 mL) was added Pd(dppf)Cl₂·CH₂Cl₂ (219.83 mg, 269.19 μmol, 0.1 eq). The mixture was stirred at 80° C. for 2 hr under N2 atmosphere. The reaction mixture was filtered and concentrated under reduced pressure to give the product. The residue was purified by flash silica gel chromatography (ISCO®; 20 g SepaFlash®-Silica Flash Column, Eluent of 0-23% Ethyl acetate/Petroleum ether gradient @ 30 mL/min). Compound 1-(oxetan-3-yl)-4-[4-(4,4,5,5-tetramethyl-1,3,2-dioxaborolan-2-yl)phenyl]piperazine (500 mg, crude) was obtained as a yellow solid.

MS m/z (ESI): 345 [M+H]⁺.

### Step 4

A mixture of 2-[1-(2-ethylsulfanyl-3,6-dimethyl-4-oxo-chromen-8-yl)ethylamino]benzoic acid (70 mg, 176.11 μmol, 1 eq), 1-(oxetan-3-yl)-4-[4-(4,4,5,5-tetramethyl-1,3,2-dioxaborolan-2-yl)phenyl]piperazine (160.1 mg, 464.92 μmol, 2.64 eq), Cs₂CO₃ (114.7 mg, 352.21 μmol, 2 eq), thiophene-2-carbonyloxy copper (67.1 mg, 352.21 μmol, 2 eq) and Pd(dppf)Cl₂·CH₂Cl₂ (28.7 mg, 35.22 μmol, 0.2 eq) in dioxane (2 mL) was stirred at 100° C. for 12 h. The reaction mixture was filtered to remove the insoluble and concentrated under vacuum to give the crude product. The residue was purified by prep-HPLC (column: Xtimate C18 100*30 mm*10 um; mobile phase: [water (F A)-ACN]; B %: 40%-60%, 10 min). Compound 2-[1-[3,6-dimethyl-2-[4-[4-(oxetan-3-yl)piperazin-1-yl]phenyl]-4-oxo-chromen-8-yl]ethylamino]benzoic acid (7.6 mg, 13.20 μmol, 7.49% yield, 96.138% purity) was obtained as a yellow solid.

¹H NMR (400 MHz, DMSO-d6) δ ppm 8.45 (br d, J=1.63 Hz, 1H) 7.81 (d, J=7.63 Hz, 1H) 7.74 (s, 1H) 7.68 (d, J=8.76 Hz, 2H) 7.49 (s, 1H) 7.20 (t, J=7.75 Hz, 1H) 7.10 (d, J=8.88 Hz, 2H) 6.54 (t, J=7.44 Hz, 1H) 6.43 (d, J=8.51 Hz, 1H) 5.09-5.19 (m, 1H) 4.53-4.60 (m, 2H) 4.45-4.51 (m, 2H) 3.39-3.53 (m, 4H) 2.45-2.48 (m, 1H) 2.41 (br d, J=4.00 Hz, 4H) 2.35 (s, 3H) 2.13 (s, 3H) 159 (d J=6.63 Hz, 3H) MS m/z (ESI): 554[M+H]⁺.

### Example 119

### 2-((1-(6-methyl-2-(2-(2-morpholinoethyl)-2H-indazol-5-yl)-4-oxo-411-chromen-8-yl)ethyl)amino)benzoic acid

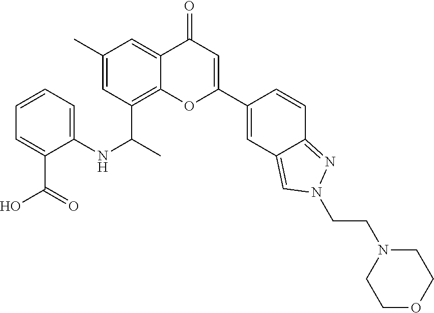 [SMILES: Cc1cc(C(C)Nc2ccccc2C(=O)O)c2oc(-c3ccc4nn(CCN5CCOCC5)cc4c3)cc(=O)c2c1]

2-((1-(6-methyl-2-(2-(2-morpholinoethyl)-2H-indazol-5-yl)-4-oxo-4H-chromen-8-yl)ethyl)amino)benzoic acid is prepared in accordance with the method of Example 1.

¹H NMR (400 MHz, DMSO-d₆) δ ppm 8.62 (br d, J=3.63 Hz, 2H) 7.86-7.94 (m, 2H) 7.74-7.78 (m, 2H) 7.56 (s, 1H) 7.22-7.29 (m, 1H) 7.03-7.11 (m, 2H) 6.40-6.52 (m, 2H) 5.31-5.38 (m, 1H) 4.61 (br t, J=6.19 Hz, 2H) 3.54 (br t, J=4.38 Hz, 4H) 2.88 (br t, J=6.32 Hz, 2H) 2.44 (br s, 4H) 2.33-2.37 (m, 3H) 1.67 (br d, J=6.50 Hz, 3H) MS m/z (ESI): 553 [M+H]⁺.

### Example 120

### 2-((1-(6-methyl-2-(2-methyl-1-oxo-1,2-dihydroisoquinolin-6-yl)-4-oxo-4H-chromen-8-yl)ethyl)amino)benzoic acid

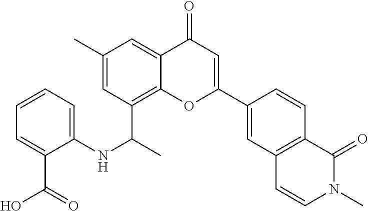 [SMILES: Cc1cc(C(C)Nc2ccccc2C(=O)O)c2oc(-c3ccc4c(=O)n(C)ccc4c3)cc(=O)c2c1]

2-((1-(6-methyl-2-(2-methyl-1-oxo-1,2-dihydroisoquinolin-6-yl)-4-oxo-4H-chromen-8-yl)ethyl)amino)benzoic acid is prepared in accordance with the method of Example 1.

¹H NMR (400 MHz, DMSO-d₆) δ ppm 8.46 (br d, J=5.00 Hz, 1H) 8.42 (d, J=1.38 Hz, 1H) 8.34 (d, J=8.51 Hz, 1H) 8.18 (dd, J=8.50, 1.63 Hz, 1H) 7.82 (dd, J=8.19, 1.44 Hz, 1H) 7.77 (d, J=1.25 Hz, 1H) 7.56-7.61 (m, 2H) 7.21-7.26 (m, 2H) 6.76 (d, J=7.25 Hz, 1H) 6.54-6.59 (m, 2H) 5.40 (quin, J=6.10 Hz, 1H) 3.53 (s, 3H) 2.38 (s, 3H) 1.70 (d, J=6.50 Hz, 3H) MS m/z (ESI): 481 [M+H]⁺.

### Example 121

### 2-((1-(6-methyl-2-(3-methyl-4-oxo-3,4-dihydroquinazolin-7-yl)-4-oxo-4H-chromen-8-yl)ethyl)amino)benzoic acid

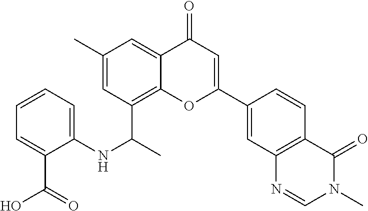 [SMILES: Cc1cc(C(C)Nc2ccccc2C(=O)O)c2oc(-c3ccc4c(=O)n(C)cnc4c3)cc(=O)c2c1]

2-((1-(6-methyl-2-(3-methyl-4-oxo-3,4-dihydroquinazolin-7-yl)-4-oxo-4H-chromen-8-yl)ethyl)amino)benzoic acid is prepared in accordance with the method of Example 1.

¹H NMR (400 MHz, DMSO-d₆) δ ppm 8.94 (br s, 1H) 8.46 (s, 1H) 838 (s, 1H) 8.27-8.32 (m, 1H) 8.22-8.26 (m, 1H) 7.83 (br d, J=7.13 Hz, 1H) 7.75 (s, 1H) 7.51-7.67 (m, ²H) 7.28 (s, 1H) 7.12 (br t, J=7.44 Hz, 1H) 6.42-6.54 (m, 2H) 5.27-5.38 (m, 1H) 3.53 (s, 3H) 2.36 (s, 3H) 1.68 (br d, J=6.50 Hz, 3H) MS m/z (ES): 482 [M+H]⁺.

### Example 122

### 2-((1-(6-methyl-2-(2-methyl-1-oxo-1,2-dihydrophthalazin-6-yl)-4-oxo-4H-chromen-8-yl)ethyl)amino)benzoic acid

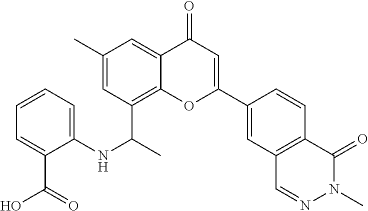 [SMILES: Cc1cc(C(C)Nc2ccccc2C(=O)O)c2oc(-c3ccc4c(=O)n(C)ncc4c3)cc(=O)c2c1]

2-((1-(6-methyl-2-(2-methyl-1-oxo-1,2-dihydrophthalazin-6-yl)-4-oxo-4H-chromen-8-yl)ethyl)amino)benzoic acid is prepared in accordance with the method of Example 1.

¹H NMR (400 MHz, DMSO-d₆) δ ppm 8.70 (d, J=1.13 Hz, 1H) 8.52-8.57 (m, 2H) 8.48 (br d, J=1.88 Hz, 1H) 8.38 (d, J=851 Hz, 1H) 7.82 (dd, J=8.13, 1.50 Hz, 1H) 7.77 (s, 1H) 7.61 (d, J=1.75 Hz, 1H) 7.29 (s, 1H) 7.20-7.26 (m, 1H) 6.51-6.62 (m, 2H) 5.42 (br t, J=6.07 Hz, 1H) 3.75 (s, 3H) 2.38 (s, 3H) 1.70 (d, J=6.63 Hz, 3H).

MS m/z (ESI): 482 [M+H]⁺.

### Example 123

### 2-[[(1R)-1-[2-[4-(4-acetylpiperazin-1-yl)phenyl]-3,6-dimethyl-4-oxo-chromen-8-yl]ethyl]amino]benzoic acid and 2-[[(1S)-1-[2-[4-(4-acetylpiperazin-1-yl)phenyl]-3,6-dimethyl-4-oxo-chromen-8-yl]ethyl]amino]benzoic acid

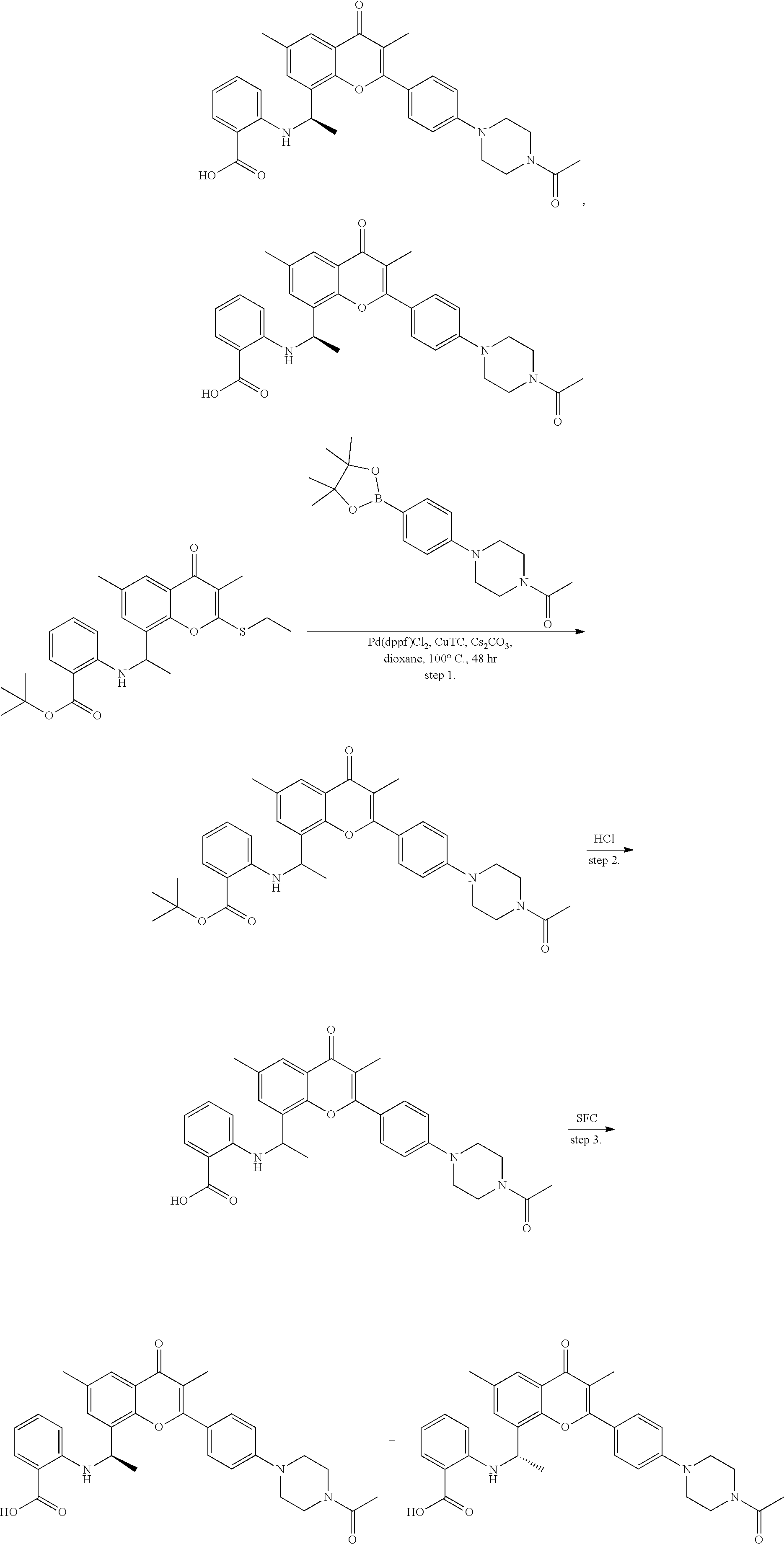

### Step 1

A mixture of tert-butyl 2-[1-(2-ethylsulfanyl-3,6-dimethyl-4-oxo-chromen-8-yl)ethyl amino]benzoate (200 mg, 440.92 μmol, 1 eq), 1-[4-[4-(4,4,5,5-tetramethyl-1,3,2-dioxaborolan-2-yl)phenyl]piperazin-1-yl]ethanone (291.2 mg, 881.85 μmol, 2 eq), Cs₂CO₃ (287.3 mg, 881.85 μmol, 2 eq), thiophene-2-carbonyloxycopper (168.16 mg, 881.85 μmol, 2 eq) and Pd(dppf)Cl₂·CH₂Cl₂? (72.02 mg, 88.18 μmol, 0.2 eq) in dioxane (10 mL) was stirred at 100° C. for 12 h. The reaction mixture was filtered to remove the insoluble and concentrated under vacuum to give the crude product. The residue was purified by flash silica gel chromatography (ISCO®; 12 g SepaFlash® Silica Flash Column, Eluent of 60-65% Ethyl acetate/Petroleum ether gradient @ 25 mL/min). Compound tert-butyl 2-[1-[2-[4-(4-acetylpiperazin-1-yl)phenyl]-3,6-dimethyl-4-oxo-chromen-8-yl]ethylamino]benzoate (130 mg, 177.54 μmol, 40% yield, 81.359% purity) was obtained as a cyan oil.

MS mm/z (ESI): 596[M+H]⁺.

### Step 2

To a solution of tert-butyl 2-[1-[2-[4-(4-acetylpiperazin-1-yl) phenyl]-3,6-dimethyl-4-oxo-chromen-8-yl]eth ylamino]benzoate (110 mg, 184.65 μmol, 1 eq) in ACN (2 mL) was added HCl (12 M, 61.5 uL, 4 eq). The mixture was stirred at 80° C. for 1 hr. The reaction mixture was filtered to remove the insoluble and concentrated under vacuum to give the crude product. The residue was purified by prep-HPLC (column: Xtimate C18 100*30 mm*10 um; mobile phase: [water (FA)-ACN]; B %: 55%-85%, 10 min). Compound 2-[1-[2-[4-(4-acetylpiperazin-1-yl) phenyl]-3,6-dimethyl-4-oxo-chromen-8-yl] ethylamino] benzoic acid (45 mg, 83.39 μmol, 45% yield) was obtained as a yellow solid.

MS m/z (ESI): 540 [M+H]⁺.

### Step 3

The stereoisomers of example 123: 2-[[(1R)-1-[2-[4-(4-acetylpiperazin-1-yl)phenyl]-3,6-dimethyl-4-oxo-chromen-8-yl]ethyl]amino]benzoic acid and 2-[[(1S)-1-[2-[4-(4-acetylpiperazin-1-yl)phenyl]-3,6-dimethyl-4-oxo-chromen-8-yl]ethyl]amino]benzoic acid

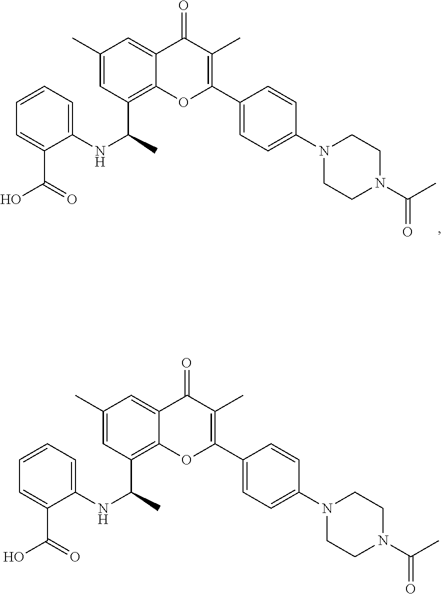 [SMILES: CC(=O)N1CCN(c2ccc(-c3oc4c([C@@H](C)Nc5ccccc5C(=O)O)cc(C)cc4c(=O)c3C)cc2)CC1 ; CC(=O)N1CCN(c2ccc(-c3oc4c([C@@H](C)Nc5ccccc5C(=O)O)cc(C)cc4c(=O)c3C)cc2)CC1]

The stereoisomers of example 123 were prepared in accordance with the method of Example 20 by chiral SFC (column: ChiralpaklB N-3, 250×30 mm I.D., 5™; mobile phase: A for CO₂ and B for ETOH (0.1% NH₃·H₂O); B %: 50%-50%, min; Flow rate: 80 mL/min; Back pressure: 100 bar; Column temperature: 40° C.; Wavelength: 220 nm) to give Peak 1 (Rt==1.80 min, 123A) and Peak 2 (Rt=2.35 min, 123B). 123A and 123B were assigned arbitrarily.

Peak 1: MS m/z (ESI): 540[M+H]⁺.

¹H NMR (400 MHz, DMSO-d6) δ ppm 9.14-9.58 (m, 1H) 7.86 (br d, J=6.88 Hz, 1H) 771 (br d, J=9.01 Hz, 3H) 7.49 (br s, 1H) 7.11 (br d, J=8.50 Hz, 2H) 6.95-7.06 (m, 1H) 6.45 (br t, J=7.19 Hz, 1H) 6.28 (br d, J=7.63 Hz, 1H) 5.10 (br s, 1H) 3.60 (br s, 4H) 3.45-3.51 (m, 4H) 2.33 (br s, 3H) 2.14 (s, 3H) 2.06 (s, 3H) 1.55 (br d, J=5.88 Hz, 3H)

Peak 2: MS m/z (ESI): 540[M+H]⁺.

¹H NMR (400 MHz, DMSO-d6) δ ppm 8.92-9.54 (m, 1H) 7.85 (br d, J=7.38 Hz, 1H) 7.64-7.77 (m, 3H) 7.49 (br s, 1H) 7.11 (br d, J=8.50 Hz, 2H) 7.05 (br s, 1H) 6.47 (br t, J=7.13 Hz, 1H) 6.30 (br d, J=775 Hz, 1H) 5.11 (br s, 1H) 3.60 (br s, 4H) 3.42-3.54 (m, 4H) 2.34 (br s, 3H) 2.14 (s, 3H) 2.06 (s, 3H) 1.56 (br d, J=6.13 Hz, 3H)

### Example 124

### 2-((1-(3,6-dimethyl-4-oxo-2-(1-(pyridin-2-yl)piperidin-4-yl)-4H-chromen-8-yl)ethyl)amino)benzoic acid

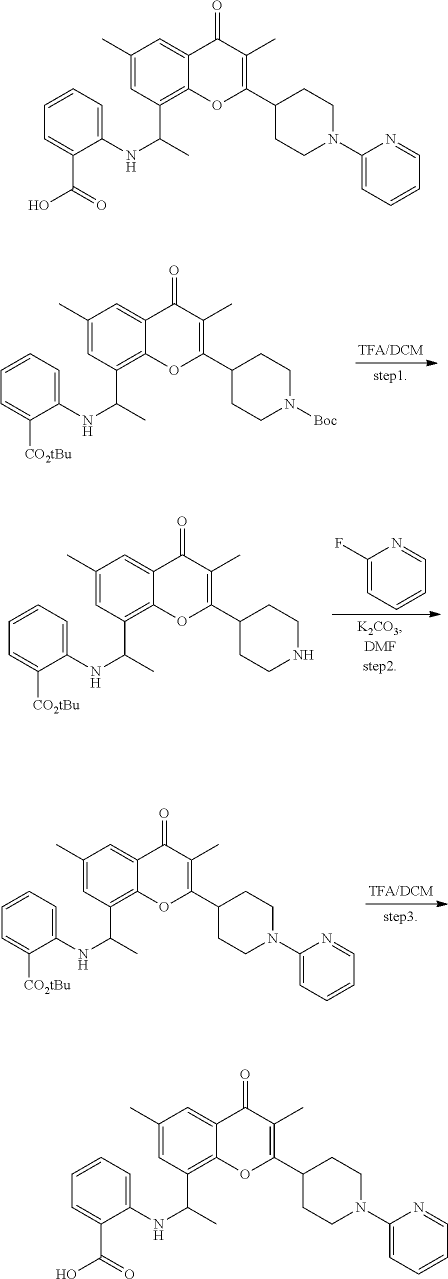 [SMILES: Cc1cc(C(C)Nc2ccccc2C(=O)O)c2oc(C3CCN(c4ccccn4)CC3)c(C)c(=O)c2c1 ; Cc1cc(C(C)Nc2ccccc2C(=O)OC(C)(C)C)c2oc(C3CCN(C(=O)OC(C)(C)C)CC3)c(C)c(=O)c2c1 ; Cc1cc(C(C)Nc2ccccc2C(=O)OC(C)(C)C)c2oc(C3CCNCC3)c(C)c(=O)c2c1 ; Fc1ccccn1 ; Cc1cc(C(C)Nc2ccccc2C(=O)OC(C)(C)C)c2oc(C3CCN(c4ccccn4)CC3)c(C)c(=O)c2c1 ; Cc1cc(C(C)Nc2ccccc2C(=O)O)c2oc(C3CCN(c4ccccn4)CC3)c(C)c(=O)c2c1]

### Step 1

To a solution of tert-butyl 4-(8-(1-((2-(tert-butoxycarbonyl)phenol)amino)ethyl)-3,6-dimethyl-4-oxo-4-chromen-2-yl)piperidine-carboxylate (244 mg, 423.08 μmol, 1 eq) in DCM (10 mL) was added dropwise TFA (0.5 mL) at 25° C. After addition, the mixture was stirred at 25° C. for 16 hr. To the reaction mixture was added NaHCO₃ (Ig) and the mixture was stirred at 25° C.; for 1 h. The Mixture was filtered and the filtrate concentrated to give crude tert-butyl 2-((1-(3,6-dim ethyl-4-oxo-2-(piperidin-4-yl)-4H-chromen-1-yl)ethyl)amino)benzoate. The residue was purified by flash silica gel chromatography to give tert-butyl 4-[8-[1-(2tert-butoxycarbonylanilino)ethyl]-6-methyl-4-oxo-chromen-2-yl]-3,6-dihydro-2H-pyridine-1-carboxylate (150 mg, 89.48% purity) as a white solid.

MS m/z (ES1): 477 [M+H]⁺.

### Step 2

To a solution of tert-butyl 4-[8-[1-(2-tert-butoxycarbonylanilino)ethyl]-6-methyl-4-oxo-chromen-2-yl]-3,6-dihydro-2H-pyridine-1-carboxylate (46 mg, 85.90 μmol, 89% purity, 1 eq) in DMF (2 mL) was added K₂CO₃ (23.74 mg, 171.80 μmol, 2 eq) and 2-fluoropyridine (41.70 mg, 429.50 μmol, 36.90 uL, 5 eq). The mixture was stirred at 120° C. for 48 hr. The mixture was concentrated in vacuo. The residue was purified by flash silica gel chromatography to give tert-butyl 2-((1-(3,6-dimethyl-4-oxo-2-(l-(pyridin-2-yl)piperidin-4-yl)-4H-chromen-8-yl)ethyl)amino)benzoate (ZXP-3216-3, 45 mg) as a white solid.

MS m/z (ESI): 554 [M+H]⁺.

### Step 3

To a solution of tert-butyl 2-((1-(3,6-dimethyl-4-oxo-2-(1-(pyridin-2-yl)piperidin-4-yl)-4-chromen-8-yl)ethyl)amino)benzoate (45 mg) in DCM (1 mL) was added TFA (1 mL). The mixture was stirred at 20° C. for 16 h. The reaction mixture was concentrated under reduced pressure. The residue was purified by prep-HPLC (column: Phenomenex luna C18 150*25 mm*10 um; mobile phase: [water (FA)-ACN]; B %: 22%-52%, 15 min) to give 2-((1-(3,6-dimethyl-4-oxo-2-(1-(pyridin-2-yl)piperidin-4-yl)-4H-chromen-8-yl)ethyl)amino)benzoic acid (2.5 mg, 4.82 μmol, 5.93% yield, 95.9% purity) as a white solid.

¹H NMR (400 MHz, DMSO-d₆) δ=8.35 (brs, 1H), 8.17-8.06 (m, 1H), 7.80 (dd, J=1.6, 8.0 Hz, 1H), 7.70 (d, J=L 2 Hz, 1H), 7.50 (t, J=7.2 Hz, 1H), 7.42 (d, J=2.0 Hz, 1H), 7.19-7.11 (m, 1H), 6.90-6.84 (m, 1H), 6.63-6.46 (m, 2H), 6.29 (d, J=8.4 Hz, 1H), 5.01 (t, J=6.4 Hz, 1H), 4.44 (br t, =: 14.0 Hz, 2H), 3.18 (m, 114), 2.94 (t, J=11.6 Hz, 2H), 2.31 (s, 3H), 2.07 (s, 3H), 1.98-1.86 (m, 4H), 1.48 (d, J=6.8 Hz, 3H) MS m/z (ESI): 498 [M+H]⁺.

### Example 125

### 2-((1-(3,6-dimethyl-4-oxo-2-(1-(pyridin-3-yl)piperidin-4-yl)-4H-chromen-8-yl)ethyl)amino)benzoic acid

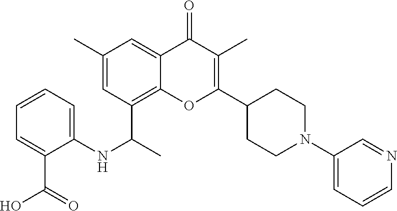 [SMILES: Cc1cc(C(C)Nc2ccccc2C(=O)O)c2oc(C3CCN(c4cccnc4)CC3)c(C)c(=O)c2c1]

2-((1-(3,6-dimethyl-4-oxo-2-(1-(pyridin-3-yl)piperidin-4-yl)-4H-chromen-8-yl)ethyl)amino)benzoic acid is prepared in accordance with the method of Example 124.

¹H NMR (400 MHz, DMSO-d₆) δ=8.48 (brs, 1H), 8.36 (d, J=2.8 Hz, 1H), 7.98 (d, J=4.0 Hz, 1H), 7.84-7.78 (m, 1H), 7.71 (s, 1H), 7.46 (d, J=2.0 Hz, 1H), 7.38 (dd, J=2.0, 8.4 Hz, 1H), 7.23-7.14 (m, 2H), 6.53 (t, J=7.6 Hz, 1H), 6.33 (d, J=8.4 Hz, 1H), 5.13-5.01 (m, 1H), 3.96-3.87 (m, 2H), 3.25 (m, 1H), 2.97-2.85 (m, 2H), 2.33 (s, 4H), 2.07 (s, 3H), 2.05-1.92 (m, 4H), 1.53 (d, J=6.8 Hz, 3H).

MS mm/z (ESI): 498 [M+H]⁺.

### Example 126

### 2-((1-(3,6-dimethyl-4-oxo-2-(1-(pyridin-4-yl) piperidin-4-yl)-4H-chromen-8-yl)ethyl)amino)benzoic acid

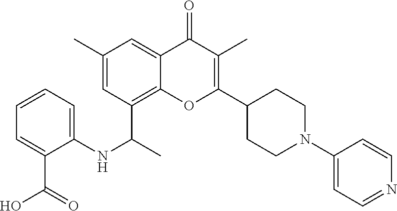 [SMILES: Cc1cc(C(C)Nc2ccccc2C(=O)O)c2oc(C3CCN(c4ccncc4)CC3)c(C)c(=O)c2c1]

2-((1-(3,6-dimethyl-4-oxo-2-(1-(pyridin-4-yl)piperidin-4-yl)-4H-chromen-8-yl)ethyl)amino)benzoic acid is prepared in accordance with the method of Example 124.

¹H NMR (400 MHz, DMSO-d₆) δ ppm 8.86 (s, 1H), 8.26 (s, 1H), 8.15 (d, J=6.0 Hz, 2H), 7.83 (dd, J=7.60, 1.20 Hz, 1H), 7.69 (s, 1H), 7.43 (d, J=2.0 Hz, 1H), 7.07 (t, J=7.20 Hz, 1H), 6.88 (d, J=6.40 Hz, 2H), 6.48 (t, J=7.20 Hz, 1H), 6.23 (d, J=8.40 Hz, 1H), 5.10-4.83 (m, 1H), 4.04-4.12 (m, 2H), 3.30-3.50 (m, 1H), 3.00-3.08 (m, 21H), 2.27-2.36 (m, 31H), 2.06-2.11 (m, 3H), 1.88-2.01 (m, 4H), 1.45 (d, J=6.80 Hz, 3H).

MS m/z (ESI): 498 [M+H]⁺.

### Example 127

### 2-((1-(3,6-dimethyl-2-(4-(methylsulfonyl)phenyl)-4-oxo-4-chromen-8-yl)ethyl)amino)benzoic acid

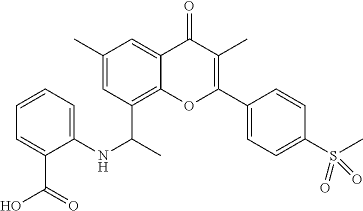 [SMILES: Cc1cc(C(C)Nc2ccccc2C(=O)O)c2oc(-c3ccc(S(C)(=O)=O)cc3)c(C)c(=O)c2c1]

2-((1-(3,6-dimethyl-2-(4-(methylsulfonyl)phenyl)-4-oxo-4H-chromen-8-yl)ethyl)amino)benzoic acid is prepared in accordance with the method of Example 123.

¹H NMR (400 MHz, DMSO-d₆) δ=9.43 (brs, 1H), 8.40 (s, 1H), 8.11 (q, J=8.4 Hz, 41H), 7.86 (dd, J=1.2, 7.6 Hz, 1H), 7.77 (s, 1H), 7.52 (d, J=1.2 Hz, 1H), 6.98 (br t, J=80 Hz, 1H), 6.43 (t, J=7.6 Hz, 1H), 6.27 (d, J=8.0 Hz, 1H), 5.06 (br t, J=6.8 Hz, 1H), 2.54 (br s, 3H), 2.34 (s, 3H), 2.09 (s, 3H), 1.53 (br d, J=6.4 Hz, 3H) MS m/z (ESI): 492 [M+H]⁺.

### Example 128

### 2-((1-(3,6-dimethyl-2-(4-(morpholinomethyl)phenyl)-4-oxo-4H-chromen-8-yl)ethyl)amino)benzoic acid

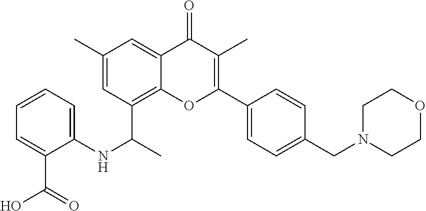 [SMILES: Cc1cc(C(C)Nc2ccccc2C(=O)O)c2oc(-c3ccc(CN4CCOCC4)cc3)c(C)c(=O)c2c1]

2-((1-(3,6-dimethyl-2-(4-(morpholinomethyl)phenyl)-4-oxo-4H-chromen-8-yl)ethyl)amino)benzoic acid is prepared in accordance with the method of Example 123.

¹H NMR (400 MHz, DMSO-d₆) δ=8.35 (d, J=6.0 Hz, 1H), 7.85-7.70 (M, 4H), 7.55-7.48 (m, 3H), 7.20 (t, J=7.6 Hz, 1H), 6.55 (t, J=7.6 Hz, 1H), 6.45 (d, J=8.4 Hz, 1H), 5.13 (t, J=6.4 Hz, 1H), 3.61 (s, 6H), 2.44 (s, 4H), 2.37 (s, 31H), 2.09 (s, 3H), 1.59 (br d, J=6.8 Hz, 3H) MS m/z (ESI): 513 [M+H]⁺.

### Example 129

### 2-((1-(3,6-dimethyl-2-(4-(morpholine-4-carbonyl)phenyl)-4-oxo-4H-chromen-8-yl)ethyl)amino)benzoic acid

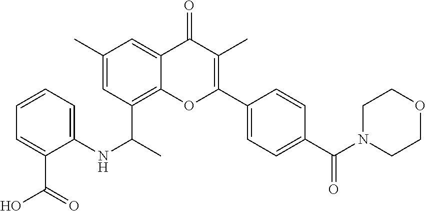 [SMILES: Cc1cc(C(C)Nc2ccccc2C(=O)O)c2oc(-c3ccc(C(=O)N4CCOCC4)cc3)c(C)c(=O)c2c1]

2-((1-(3,6-dimethyl-2-(4-(morpholine-4-carbonyl)phenyl)-4-oxo-4H-chromen-8-yl)ethyl)amino)benzoic acid is prepared in accordance with the method of Example 123.

¹H NMR (400 MHz, DMSO-d₆) δ=8.53 (brs, 1H), 7.88 (d, J=8.4 Hz, 2H), 7.84-7.78 (m, 2H), 7.62 (d, J=8.0 Hz, 2H), 7.54 (d, J=1.8 Hz, 1H), 7.18 (br t, J=7.6 Hz, 1H), 6.53 (t, J=7.6 Hz, 1H), 6.44 (d, J=8.4 Hz, 1H), 5.13 (s, 1H), 3.56 (s, 8H), 2.37 (s, 3H), 2.11 (s, 3H), 1.59 (br d, J=6.4 Hz, 3H)

MS m/z (ESI): 527 [M+H]⁺.

### Example 130

### 2-((1-(3,6-dimethyl-4-oxo-2-(4-phenylcyclohexyl)-411-chromen-8-yl)ethyl)amino)benzoic acid

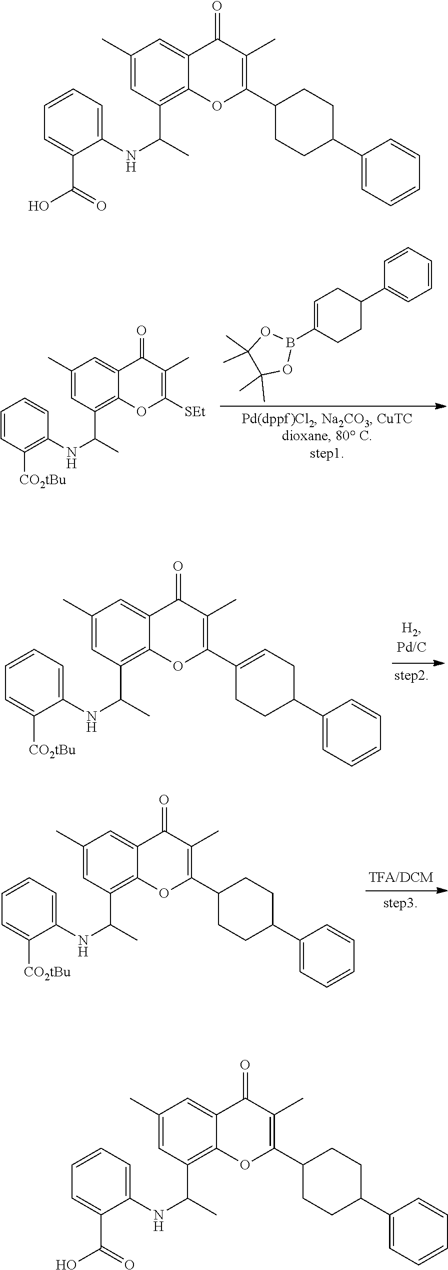

### Step 1

A mixture of tert-buty (2(1-(2-(ethylthio)-3,6-dimethyl-4-oxo-4-1-chromen-8-yl)ethyl)amino)benzoate (50 mg, 110.23 μmol), 4,4,5,5-tetramethyl-2-(4-phenylcyclohexen-1-yl)-1,3,2-dioxaborolane (62.66 mg. 220.46 μmol), CuTC (50.01 mg, 262.28 μmol), Pd(dppf)Cl₂ (80.02 mg, 109.36 μmol) and Cs₂CO₃ (110.03 mg, 337.71 μmol) in dioxane (10 mL) was degassed and purged with N₂ for 3 times, and then the mixture was stirred at 80° C. for 16 h under N₂ atmosphere. The reaction mixture was filtered and the filtrate was concentrated to dryness to give a residue. The residue was purified by prep-TLC (Petroleum ether:Ethyl acetate=2:1) to give tert-butyl 2-((1-(3,6-dimethyl-4-oxo-2-(1,2,3,6-tetrahydro-[1,1′-biphenyl]-4-yl)-41-H-chromen-8-yl)ethyl)amino)benzoate (30 mg, crude) as a white solid.

MS m/z (ESI): 550 [M+H]⁺.

### Step 2

To a solution of tert-butyl 2-((1-(3,6-dimethyl-4-oxo-2-(1,2,3,6-tetrabydro-[1,1′-biphenyl]-4-yl)-4H-chromen-8-yl)ethyl)amino)benzoate (30 mg, 54.58 μmol) in THF (1 mL) and EtOH (1 mL) was added Pd/C (12.00 mg, 60.00 μmol, 10% purity). The mixture was stirred at 25° C. for 0.5 hr tinder H₂ atmosphere. The reaction mixture was filtered and concentrated tinder reduced pressure to give crude tert-butyl 2-((1-(3,6-dimethyl-4-oxo-2-(4-phenylcyclohexyl)-4H-chromen-8-yl)ethyl)amino)benzoate (10 mg) as a white solid. (LCMS: EC8398-62-P1A2). MS m/z (ESI): 550 [M+H]⁺.

### Step 3

A mixture of tert-butyl 2-((1-(3,6-dimethyl-4-oxo-2-(4-phenylcyclohexyl)-4H-chromen-8-yl)ethyl)amino)benzoate (10 mg, 71.09 μmol) in DCM (1 mL) and TFA (909 uL) was degassed and purged with N₂ for 3 times, and then the mixture was stirred at 25° C. for 16 hr under N₂ atmosphere. The reaction mixture was filtered and concentrated under reduced pressure to give a residue. The residue was purified by prep-H-PLC (column: Phenomenex luna C18 150*25 mm*10 um; mobile phase: [water (FA)-ACN]; B %: 69%-89%, 58 min). to give 2-[l-[3,6-dimethyl-4-oxo-2-(4-phenylcyclohexyl)chromen-8-yl]ethylamino]benzoic acid (1.8 mg, 3.62 μmol, 19.97% yield, 99.65% purity) as a white solid.

¹H NMR (400 MHz, DMSO-d₆) δ ppm 8.54 (brs, 1H), 7.83 (d, J=7.6 Hz, 1H), 7.69 (s, 1H), 7.40 (d, J=1.6 Hz, 1H), 7.32 (d, J=7.2 Hz, 2H), 7.24-6.98 (m, 4H), 6.54 (t, J=7.6 Hz, 1H), 6.19 (d, J=8.4 Hz, 1H), 4.94 (s, 1H), 3.03-2.82 (m, 2H), 2.30 (s, 3H), 2.25-2.11 (m, 2H), 2.09-2.05 (m, 1H), 2.03 (s, 3H), 2.02-1.77 (n, 5H), 1.37 (d, J=6.4 Hz, 3H).

MS m/z (ESI): 496 [M+H]⁺.

### Example 131

### 2-((1-(3,6-dimethyl-2-(2-methyl-2H-indazol-5-yl)-4-oxo-4H-chromen-8-yl)ethyl)amino)benzenesulfonamide

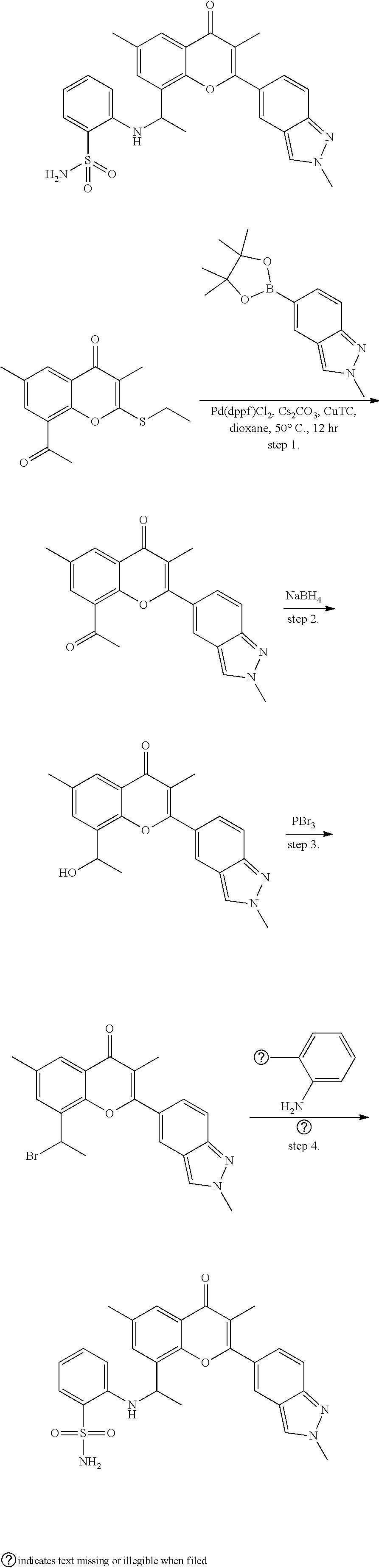

### Step 1

A mixture of 8-acetyl-2-ethylsulfanyl-3,6-dimethyl-chromen-4-one (900 mg, 3.26 mmol, 1 eq), 2-methyl-5-(4,41,5,5-tetramethyl-1,3,2-dioxaborolan-2-yl)indazole (1.01 g, 3.91 mmol, 1.2 eq), Cs₂CO₃ (212 g, 6.52 mmol, 2 eq), thiophene-2-carbonyloxycopper (2.49 g, 13.04 mmol, 4 eq) and Pd(dppf)Cl₂·CH₂Cl₂ (1.06 g, 1.30 mmol, 0.4 eq) in dioxane (30 mL) was stirred at 50° C. for 12 h. The reaction mixture was diluted with EtOAc (200 mL) and filtered to remove the insoluble and concentrated under vacuum to give a residue. The residue was purified by flash silica gel chromatography (ISCO®; 20 g SepaFlash® Silica Flash Column, Eluent of 0-70% Ethyl acetate/Petroleum ether gradient @ 30 mL/min). Compound 8-acetyl-3,6-dimethyl-2-(2-methylindazol-5-yl)chromen-4-one (450 ng, 1.30 mmol, 39.85% yield) was obtained as a white solid.

MS m/z (ESI): 347 [M+H]⁺.

### Step 2

To a solution of 8-acetyl-3,6-dimethyl-2-(2-methylindazol-5-yl)chromen-4-one (300 mg, 866.10 μmol, 1 eq) in MeOH (5 mL) was added NaBH₄ (220.28 mg, 1.04 mmol, 1.2 eq) in portions at 0° C. The mixture was stirred at 25° C. for 1 hr. The mixture was quenched with sat. NH₄Cl (20 mL) and extracted with EtOAc (40 mL*3). The organic layer dried over Na₂SO₄, filtered and under vacuum to give crude product. The crude product was without purification. Compound 8-(1-hydroxyethyl)-3,6-dimethyl-2-(2-methylindazol-5-yl)chromen-4-one (400 mg, crude) was obtained as a white solid.

MS m/z (ESI): 349 [M+H]⁺.

### Step 3

To a solution of 8-(1-hydroxyethyl)-3,6-dimethyl-2-(2-methylindazol-5-yl)-chromen-4-one (350 mg, 1.00 mmol, 1 eq) in DCM (5 mL) was added PBr₃ (271.9 mg, 1.00 mmol, 0.7 mL, 1 eq). The mixture was stirred at 25° C. for 3 hr. The reaction mixture was quenched by addition sat.NaHCO₃ (20 mL) at 0° C., and then diluted with DCM (10 mL) and extracted with DCM (20 mL*3). The combined organic layers dried over Na₂SO₄, filtered and concentrated under reduced pressure to give crude product. The crude product was without purification. Compound 8-(1-bromoethyl)-3,6-dimethyl-2-(2-methylindazol-5-yl)chromen-4-one (400 mg, crude) was obtained as a white solid.

MS m/z (ESI): 413 [M+H]⁺.

### Step 4

A mixture of 8-(1-bromoethyl)-3,6-dimethyl-2-(2-methylindazol-5-yl)chromen-4-one (20 ng, 48.63 μmol, 1 eq), 2-aminobenzenesulfonamide (10.1 mg, 58.36 μmol, 1.2 eq) and TBAB (39.2 mg, 121.58 μmol, 2.5 eq) in ACN (1 mL) was heated at 90° C. for 12 h. The reaction mixture was filtered to remove the insoluble and concentrated under vacuum to give the crude product. The residue was purified by prep-HPLC (column: Welch Xtimate C18 150*30 mm*5 um; mobile phase: [water (F A)-ACN]; 13%: 24%-64%, 25 min). Compound 2-[1-[3,6-dimethyl-2-(2-methylindazol-5-yl)-4-oxo-chromen-8-yl]ethylamino]benzenesulfonamide (5.1 mg, 9.52 μmol, 19% yield, 93.807% purity) was obtained as a white solid.

¹H NMR (400 MHz, DMSO-d's) δ ppm 8.54 (s, 1H) 8.23 (s, 1H) 7.77 (br d, J=13.88 Hz, 2H) 7.60-7.68 (m, 3H) 7.55 (s, 2H) 7.20 (t, J=7.82 Hz, 1H) 6.66 (t, J=7.44 Hz, 1H) 6.47 (d, J=8.50 Hz, 1H) 6.36 (d, J=5.63 Hz, 1H) 5.17 (br t, J=6.19 Hz, 1H) 4.23 (s, 3H) 2.36 (s, 3H) 2.15 (s, 3H) 1.60 (d, J=6.50 Hz, 3H)

MS m/z (ESI): 503 [M+H]⁺.

### Example 132

### 2-((1-(2-(4-(1,1-dioxidothiomorpholino)phenyl)-3,6-dimethyl-4-oxo-4H-chromen-8-yl)ethyl)amino)benzoic acid

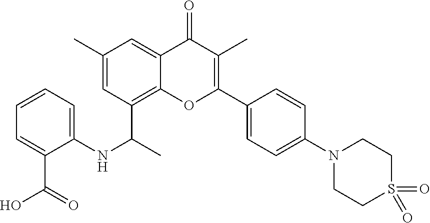 [SMILES: Cc1cc(C(C)Nc2ccccc2C(=O)O)c2oc(-c3ccc(N4CCS(=O)(=O)CC4)cc3)c(C)c(=O)c2c1]

2-((1-(2-(4-(1,1-dioxidothiomorpholino)phenyl)-3,6-dimethyl-4-oxo-4H-chromen-8-yl)ethyl)amino)benzoic acid is prepared in accordance with the method of Example 123.

¹H NMR (400 MHz, DMSO-d6) δ ppm 8.55 (s, 1H), 7.89 (d, J=7.60 Hz, 1H), 7.73-7.84 (m, 3H), 7.57 (s, 1H), 7.20-7.32 (m, 3H), 6.60 (t, J=7.60 Hz, 1H), 6.48 (br d, J=8.40 Hz, 1H), 5.21 (brs, 1H), 3.94-4.04 (m, 4H), 3.10-3.23 (m, 4H), 2.41 (s, 3H), 2.20 (s, 3H), 1.65 (d, J=6.4 Hz, 3H).

MS m/z (ESI): 547 [M+H]⁺.

### Example 133

### (R)-2-((1-(3,6-dimethyl-2-(2-morpholinopyridin-4-yl)-4-oxo-411-chromen-8-yl)ethyl)amino)benzoic acid

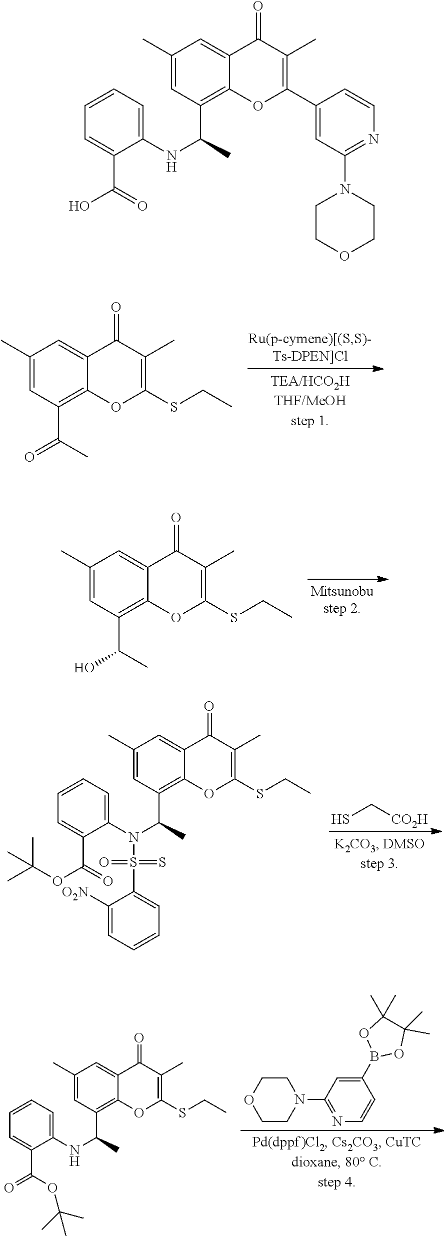

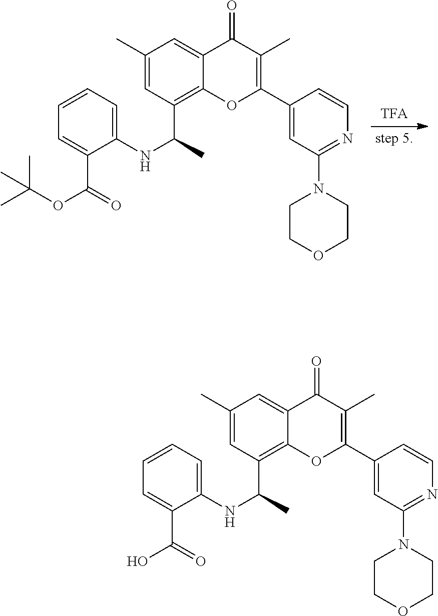 [SMILES: Cc1cc([C@@H](C)Nc2ccccc2C(=O)OC(C)(C)C)c2oc(-c3ccnc(N4CCOCC4)c3)c(C)c(=O)c2c1 ; Cc1cc([C@@H](C)Nc2ccccc2C(=O)O)c2oc(-c3ccnc(N4CCOCC4)c3)c(C)c(=O)c2c1]

### Step 1

To a solution of 8-acetyl-2-ethylsulfanyl-3,6-dimethyl-chromen-4-one (3.5 g, 12.67 mmol, 1 eq) and N-[(1S,2S)-2-amino-1,2-diphenyl-ethyl]-4-methyl-benzenesulfonamide; chlororuthenium; 1-isopropyl-4-methyl-benzene (403.5 mg, 633.25 μmol, 0.05 eq) in TI-IF (30 mL) was added a solution of TEA (3.84 g, 38.00 mmol, 5.29 mL, 3 eq) and FORMIC ACID (2.91 g, 63.33 mmol, 2.39 mL, 5 eq) in THE (30 mL). The mixture was stirred at 60° C. for 4 hr. LCMS showed desired mass was detected. The reaction mixture was concentrated under vacuum to give the crude product. The residue was purified by flash silica gel chromatography (ISCO®; 40 g SepaFlash® Silica Flash Column, Eluent of 0˜5% Ethyl acetate/Petroleum ether gradient @ 40 mL/min) to give compound 2-ethylsulfanyl-8-[(1S)-1-hydroxyethyl]-3,6-dimethyl-chromen-4-one (3.4 g, 12.21 mmol, 96% yield) as a yellow solid.

MS mm/z (ESI): 279[M+H]⁺.

### Step 2

To a solution of 2-ethylsulfanyl-8-[(1S)-1-hydroxyethyl]-3,6-dimethyl-chromen-4-one (1.8 g, 6.47 mmol, 1 eq) and tert-butyl 2-[(2-nitrophenyl)sulfonylamino]benzoate (2.69 g, 7.11 mmol, 1.1 eq) in MeCN (80 mL) was added PPh₃ (3.39 g, 12.93 mmol, 2 eq) and stirred for 0.5 hr. Then DIAD (2.62 g, 12.93 mmol, 2.51 mL, 2 eq) was added at 0° C. The mixture was stirred at 25° C. for 12 hr. LCMS showed desired mass was detected. The reaction mixture was concentrated under vacuum to give the crude product. The residue was purified by flash silica gel chromatography (ISCO®; 80 g SepaFlash® Silica Flash Column, Eluent of 0˜50% Ethyl acetate/Petroleum ether gradient @ 50 mL/min) to give compound tert-butyl 2-[[(1R)-1-(2-ethylsulfanyl-3,6-dimethyl-4-oxo-chromen-8-yl)ethyl]-(2-nitrophenyl)sulfonyl-amino]benzoate (3 g, 4.70 mmol, 72% yield) was obtained as a white solid.

MS m/z (ESI): 639 [M+H]⁺.

### Step 3

To a solution of tert-butyl 2-[[(1R)-1-(2-ethylsulfanyl-3,6-dimethyl-4-oxo-chromen-8-yl) ethyl]-(2-nitrophenyl)sulfonyl-amino]benzoate (1 g, 1.57 mmol, 1 eq) in DMF (10 mL) was added LiOH·H₂O (262.7 mg, 6.26 mmol, 4 eq). Then ethanethioic S-acid (238.3 mg, 3.13 mmol, 222.74 uL, 2 eq) was added under N₂ atmosphere. The mixture was stirred at 80° C. for 4 hr. LCMS showed desired mass was detected. The reaction mixture was added water (20 ml) and extracted with ethyl acetate (30 ml*3). The combined organic layers were dried over Na₂SO₄, filtered and concentrated under reduced pressure to give a crude product. The residue was purified by flash silica gel chromatography (ISCO®; 12 g SepaFlash® Silica Flash Column, Eluent of 0-5% Ethyl acetate/Petroleum ether gradient @ 25 mL/min) to give compound tert-butyl 24[[(1R)-1-(2-ethylsulfanyl-3,6-dimethyl-4-oxo-chromen-8-yl)ethyl]amino]benzoate (300 mg, 661.39 μmol, 42% yield) was obtained as a yellow solid.

MS m/z (ESI): 453 [M+H]⁺

### Step 4

A mixture of tert-butyl (R)-2-((1-(2-(ethylthio)-3,6-dimethyl-4-oxo-4H-chromen-8-yl)ethyl)amino)benzoate (50 mg, 110.23 μmol, 1 eq), 4-[4-(4,4,5,5-tetramethyl-1,3,2-dioxaborolan-2-yl)-2-pyridyl]morpholine (95.96 mg, 330.69 μmol, 3 eq), Pd(dppf)Cl₂ (120.99 mg, 165.35 μmol, 1.5 eq), Cs₂CO₃ (107.75 mg, 330.69 μmol, 3 eq) and CuTC (52.55 mg. 275.58 mol, 2.5 eq) in dioxane (2 mL) was degassed and purged with N₂ for 3 times, and then the mixture was stirred at 80° C. for 16 h under N₂ atmosphere. The suspension was filtered through a pad of Celite or silica gel and the pad or filter cake was washed with EA (50 mL×2). The combined filtrates were concentrated to dryness. The residue was purified by prep-TLC (SiO₂, PE:EtOAc=3:1) tert-butyl (R)-2-[1-[3,6-dimethyl-2-(2-morpholino-4-pyridyl)-4-oxo-chromen-8-yl]ethylamino] benzoate (61 mg, 109.78 μmol, 99.59% yield) was obtained as a white solid.

MS m/z (ESI): 556 [M+H]⁺.

### Step 5

A mixture of tert-butyl (R)-2-[1-[3,6-dimethyl-2-(2-morpholino-4-pyridyl)-4-oxo-chromen-8-yl]ethylamino]benzoate (60 mg. 107.981 μmol, 1 eq) and TFA (1 mL) in DCM (1 mL) was degassed and purged with N₂ for 3 times, and then the mixture was stirred at 25° C. for 16 hr under N₂ atmosphere. The reaction mixture was concentrated under reduced pressure to remove solvent. The residue was purified by prep-HPLC (column: Phenomenex luna C18 150*25 mm*10 um; mobile phase: [water (FA)-ACN]; B %: 41%-71%, 10 min). R-2-[1-[3,6-dimethyl-2-(2-morpholino-4-pyridyl)-4-oxo-chromen-8-yl]ethylamino]benzoic acid (23 mg, 46.04 μmol, 42.64% yield) was obtained as a white solid.

¹H NMR (DMSO, 400 MHz) δ_{H}=8.63 (br s, 1H), 834 (d, J=5.2 Hz, Ili), 7.82 (br d, J=8.0 Hz, 1H), 7.78 (s, 1H), 7.54 (s, 1H), 7.20-7.12 (m, 2H), 7.04 (d, J=4.8 Hz, 1H), 6.52 (t, J=7.6 Hz, 1H), 6.41 (br d, J=8.4 Hz, 1H), 5.15-5.03 (m, 1H), 3.75-3.68 (m, 4H), 3.55-3.50 (m, 4H), 2.37 (s, 3H), 2.09 (s, 3H), 1.58 (br d, J=6.8 Hz, 31H).

MS mm/z (ESI): 500 [M+H]⁺.

### Example 134

### (R)-2-((1-(2-(2-(dimethylcarbamoyl)pyridin-4-yl)-3,6-dimethyl-4-oxo-4H-chromen-8-yl)ethyl)amino)benzoic acid

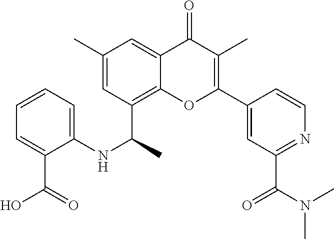 [SMILES: Cc1cc([C@@H](C)Nc2ccccc2C(=O)O)c2oc(-c3ccnc(C(=O)N(C)C)c3)c(C)c(=O)c2c1]

(R)-2-((1-(2-(2-(dimethylcarbamoyl)pyridin-4-yl)-3, 6-dimethyl-4-oxo-4H-chromen-8-yl)ethyl)amino)benzoic acid is prepared in accordance with the method of Example 133.

¹H NMR (400 MHz, DMSO-d₆) δ ppm 8.83 (d, J=4.4 Hz, 1H), 8.46 (s, 1H), 7.95 (s, 1H), 7.85-7.90 (m, 1H), 7.76-7.83 (m, 2H), 7.55 (s, 1H), 7.17 (t, J=7.20 Hz, 1H), 6.53 (t, J=7.2 Hz, 1H), 6.45 (t, J=8.8 Hz, 1H), 5.08-5.20 (m, 1H), 3.05 (s, 3H), 3.00 (s, 3H), 2.37 (s, 3H), 2.11 (s, 3H), 1.59 (br d, J=6.40 Hz, 3H).

MS m/z (ESI): 486 [M+H]⁺.

### Example 135

### (R)-2-((1-(3,6-dimethyl-4-oxo-2-(4-((2-oxopyrrolidin-1-yl)methyl)phenyl)-4H-chromen-8-yl)ethyl)amino)benzoic acid

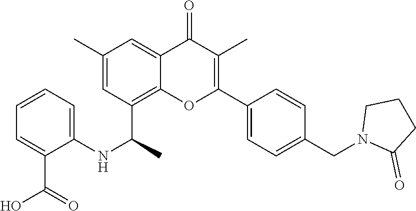 [SMILES: Cc1cc([C@@H](C)Nc2ccccc2C(=O)O)c2oc(-c3ccc(CN4CCCC4=O)cc3)c(C)c(=O)c2c1]

(R)-2-((1-(3,6-dimethyl-4-oxo-2-(4-((2-oxopyrrolidin-1-yl)methyl)phenyl)-4H-chromen-8-yl)ethyl)amino)benzoic acid is prepared in accordance with the method of Example 133.

¹H NMR (400 MHz, ACETONITRILE-d₃) δ ppm 8.35 (br d, J=6.13 Hz, 1H) 7.74-7.83 (m, 4H) 7.52 (d, J=2.00 Hz, 1H) 7.42 (d, J=8.25 Hz, 2H) 7.20 (s, 1H) 6.54 (s, 1H) 6.45 (d, J=8.50 Hz, 1H) 5.06-5.18 (m, 1H) 4.48 (s, 2H) 3.30 (br s, 2H) 2.36 (s, 3H) 2.33 (t, J=8.19 Hz, 2H) 2.09 (s, 3H) 1.97 (br t, J=7.50 Hz, 2H) 1.58 (d, J=6.63 Hz, 3H) MS m/z (ESI): 510 [M+H]⁺.

### Example 136

### (R)-2-((1-(2-(4-((1H-1,2,4-triazol-1-yl)methyl)phenyl)-3,6-dimethyl-4-oxo-411-chromen-8-yl)ethyl)amino)benzoic acid

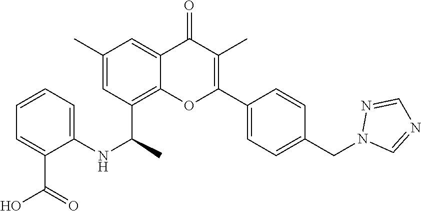 [SMILES: Cc1cc([C@@H](C)Nc2ccccc2C(=O)O)c2oc(-c3ccc(Cn4cncn4)cc3)c(C)c(=O)c2c1]

((R)-2-((1-(2-(4-((1H-1,2,4-triazol-1-yl)methyl)phenyl)-3,6-dimethyl-4-oxo-4H-chromen-8-yl)ethyl)amino)benzoic acid is prepared in accordance with the method of Example 133.

¹H NMR (400 MHz, ACETONITRILE-d₃) δ ppm 12.45-12.99 (m, 1H) 8.35 (s, 1H) 8.27 (br s, 1H) 7.74-7.83 (m, 2H) 7.51 (d, J=2.00 Hz, 1H) 7.19-7.25 (m, 3H) 7.10-7.16 (m, 1H) 7.06 (dd, J=6.82, 2.44 Hz, 2H) 6.53 (t, J=7.63 Hz, 1H) 6.26 (d, J=8.88 Hz, 1H) 5.65 (s, 2H) 4.74 (s, 1H) 2.35 (s, 3H) 1.94 (s, 3H) 1.32 (d, J=6.50 Hz, 3H)

MS m/z (ES1): 495 [M+H]⁺.

### Example 137

### (R)-2-((1-(2-(4-((4-acetylpiperazin-1-yl)methyl)phenyl)-3,6-dimethyl-4-oxo-4H-chromen-8-yl)ethyl)amino)benzoic acid

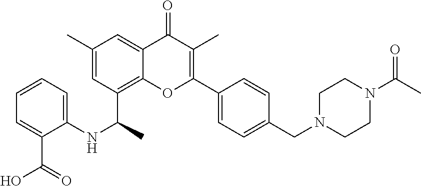 [SMILES: CC(=O)N1CCN(Cc2ccc(-c3oc4c([C@@H](C)Nc5ccccc5C(=O)O)cc(C)cc4c(=O)c3C)cc2)CC1]

(R)-2-((1-(2-(4-((4-acetylpiperazin-1-yl)methyl)phenyl)-3,6-dimethyl-4-oxo-4H-chromen-8-yl)ethyl)amino)benzoic acid is prepared in accordance with the method of Example 133.

¹H NMR (400 MHz, DMSO-d₆) δ ppm 8.48-8.74 (m, 1H) 7.75-7.84 (m, 4H) 7.53 (br d, 0.1=7.50 Hz, 3H) 7.12-7.19 (m, 1H) 6.52 (br t, J=7.32 Hz, 1H) 6.41 (br d, J=8.13 Hz, 1H) 6.37-6.38 (m, 1H) 5.12 (br d, J=2.88 Hz, 1H) 3.60 (s, 2H) 3.45 (br s, 4H) 2.42 (br s, 2H) 2.36 (s, 3H) 2.32-2.36 (m, 2H) 2.10 (s, 3H) 1.99 (s, 3H) 1.58 (br d, J=6.38 Hz, 3H) MS m/z (ESI): 554 [M+H]⁺.

### Example 138

### 2-(((1R)-1-(2-(4-(1-(4-acetylpiperazin-1-yl)ethyl)phenyl)-3,6-dimethyl-4-oxo-411-chromen-8-yl)ethyl)amino)benzoic acid

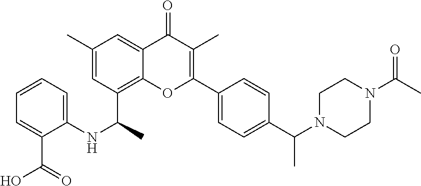 [SMILES: CC(=O)N1CCN(C(C)c2ccc(-c3oc4c([C@@H](C)Nc5ccccc5C(=O)O)cc(C)cc4c(=O)c3C)cc2)CC1]

2-(((1R)-1-(2-(4-(1-(4-acetylpiperazin-1-yl)ethyl)phenyl)-3,6-dimethyl-4-oxo-4H-chromen-8-yl)ethyl)amino)benzoic acid is prepared in accordance with the method of Example 133.

¹H NMR (400 MHz, DMSO-d₆) δ ppm 8.35-8.46 (m, 1H) 7.78 (br d, J=6.75 Hz, 4H) 7.49-7.55 (m, 3H) 7.14-7.23 (m, 1H) 6.51-6.59 (m, 1H) 6.42-6.47 (m, 1H) 5.10-5.19 (m, 1H) 3.55-3.61 (m, 1H) 3.42 (br d, J=4.63 Hz, 8H) 2.37 (s, 3H) 2.11 (s, 3H) 1.95 (s, 3H) 1.60 (br d, J=6.50 Hz, 3H) 1.36 (d, J=6.63 Hz, 3H)

MS m/z (ESI): 568 [M+H]⁺.

### Example 139

### (R)-2-((1-(2-(4-(4-acetylpiperazine-1-carbonyl)phenyl)-3,6-dimethyl-4-oxo-4H-chromen-8-yl)ethyl)amino)benzoic acid

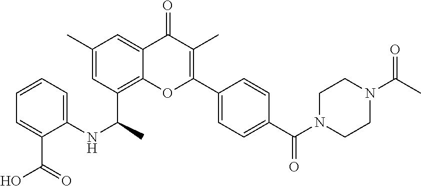 [SMILES: CC(=O)N1CCN(C(=O)c2ccc(-c3oc4c([C@@H](C)Nc5ccccc5C(=O)O)cc(C)cc4c(=O)c3C)cc2)CC1]

(R)-2-((1-(2-(4-(4-acetylpiperazine-1-carbonyl)phenyl)-3,6-dimethyl-4-oxo-4H-chromen-8-yl)ethyl)amino)benzoic acid is prepared in accordance with the method of Example 133.

¹H NMR (400 MHz, DMSO-d₆) δ ppm 7.88 (d, J=8.25 Hz, 2H) 7.76-7.83 (m, 2H) 7.62 (d, J==8.25 Hz, 2H) 7.53 (d, J=0.88 Hz, 1H) 7.18 (br t, J=6.50 Hz, 1H) 6.49-6.56 (m, 1H) 6.43 (br d, J=8.25 Hz, 1H) 5.07-5.16 (m, 1H) 3.42-3.74 (m, 8H) 2.36 (s, 3H) 2.10 (s, 3H) 2.03 (br d, J=3.50 Hz, 3H) 1.58 (br d, J=6.63 Hz, 3H) MS m/z (ESI): 510 [M+H]⁺.

### Example 140

### (R)-2-((1-(2-(4-(2-(dimethylamino)ethoxy)phenyl)-3,6-dimethyl-4-oxo-41-chromen-8-yl)ethyl)amino)benzoic acid

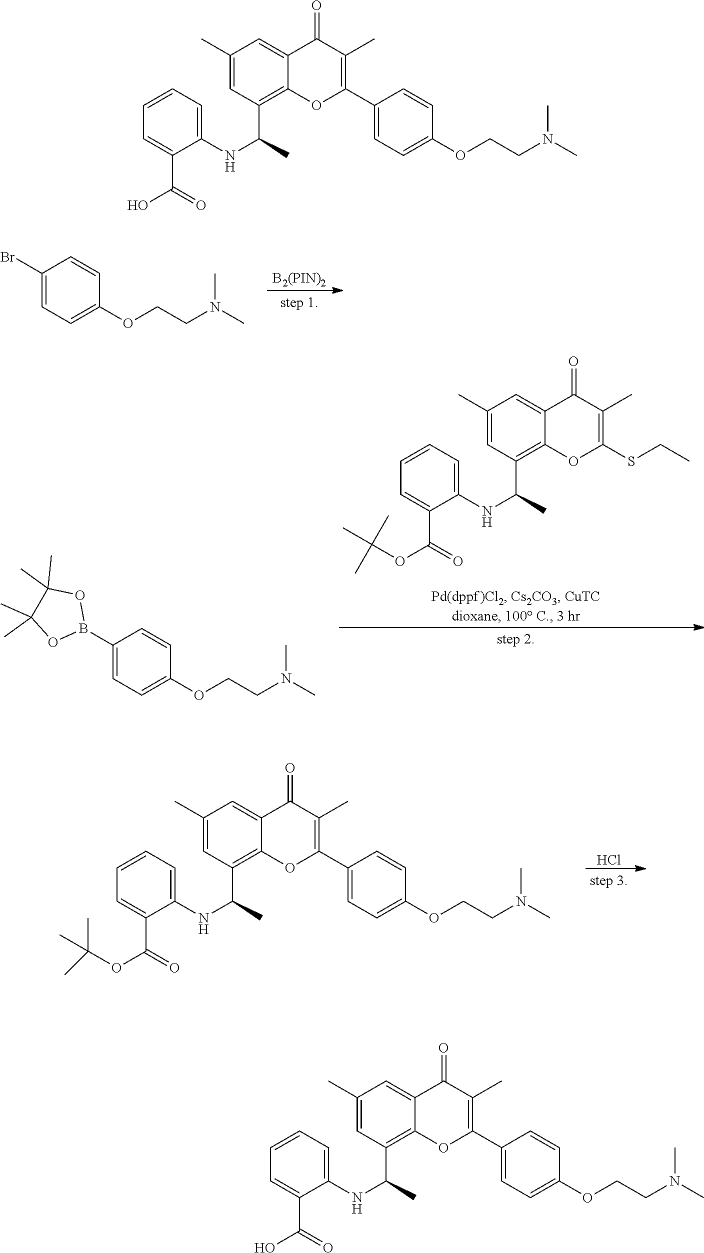

### Step 1

To a solution of 2-(4-bromophenoxy)-N,N-dimethyl-ethanamine (200 mg, 819.24 umol, 1 eq) and 4,4,5,5-tetramethyl-2-(4,4,5,5-tetramethyl-1,3,2-dioxaborolan-2-yl)-1,3,2-dioxaborolane (312 mg, 1.23 mmol, 1.5 eq) in dioxane (5 mL) were added cyclopentyl(diphenyl)phosphane; dichloromethane; dichloropalladium; iron (67 mg, 81.92 umol, 0.1 eq) and KOAc (241 mg, 2.46 mmol, 3 eq). The mixture was stirred at 100° C. for 3 h. The mixture was filtered and evaporated under vacuum to give a residue. The residue was purified by column chromatography (SiO2, Petroleum ether/Ethyl acetate=i/0 to 0/1). Compound N,N-dimethyl-2-[4-(41,4,5,5-tetramethyl-1,3,2-dioxaborolan-2-yl)phenoxy]ethanamine (200 mg, 686.83 umol, 84% yield) was obtained as a brown oil.

MS m/z (ESI): 292[M+H]⁺.

### Step 2

A mixture of N,N-dimethyl-2-[4-(4,4,5,5-tetramethyl-1,3,2-dioxaborolan-2-yl) phenoxy]ethanamine (64 mg, 220.46 umol, 2 eq), tert-butyl 2-[[(1R)-1-(2-ethylsulfanyl-3,6-dimethyl-4-oxo-chromen-8-yl)ethyl]amino]benzoate (50 mg, 110.23 umol, 1 eq), Cs₂CO₃ (72 mg, 220.46 umol, 2 eq), thiophene-2-carbonyloxycopper (42 mg, 220.46 umol, 2 eq) and Pd(dppf)Cl₂·CH₂Cl₂ (18 mg, 22.05 umol, 0.2 eq) in dioxane (2 mL) was stirred at 100° C. for 16 h under N₂. The filtrate was evaporated under vacuum to give a crude. Compound tert-butyl 2-[[(R)-1-[2-[4-[2-(dimethylamino)ethoxy]phenyl]-3,6-dimethyl-4-oxo-chromen-8-yl]ethyl]amino]benzoate (100 mg, crude) was obtained as a brown oil.

MS m/z (ESI): 557[M+H]⁺.

### Step 3

HCl (12 M, 29.94 uL, 2 eq) was added to a mixture of tert-butyl 2-[[(1R)-1-[2-[4-[2-(dimethylamino)ethoxy]phenyl]-3,6-dimethyl-4-oxo-chromen-8-yl]ethyl]amino]benzoate (100 mg, 179.63 umol, 1 eq) in MeCN (1 mL). The mixture was stirred at 80° C. for 1 h. The solvent was evaporated under vacuum to give a crude. The crude was purified by prep-HPLC (column: Welch Xtimate C18 150*30 mm*5 um; mobile phase: [water (FA)-ACN]; B %: 0%-38%, 30 min). Compound 2-[[(1R)-1-[2-[4-[2-(dimethylamino) ethoxy] phenyl]-3,6-dimethyl-4-oxo-chromen-8-yl]ethyl]amino]benzoic acid (15 mg, 28.95 umol, 16.11% yield, 96.6% purity) was obtained as a white solid.

¹H NMR (400 MHz, DMSO-d₆) δ ppm 8.44 (br s, 1H) 8.16 (s, 1H) 7.81 (d, J=7.75 Hz, 1H) 7.75 (br d, J=8.38 Hz, 3H) 7.51 (d, J=1.50 Hz, 1) 7.19 (t, J=7.75 Hz, 1H) 7.14 (d, J=8.76 Hz, 2H) 6.54 (t, J=7.50 Hz, 1H) 6.44 (d, 1=8.50 Hz, 1H) 5.12 (br s, 1H) 4.18 (t, J=5.63 Hz, 2H) 2.75 (br t, J=5.50 Hz, 2H) 2.36 (s, 31H) 2.30 (s, 6H) 2.10 (s, 3H) 1.58 (d, J=6.63 Hz, 3H) MS m/z (ESI): 501[M+H]⁺.

### Example 141

### (R)-2-((1-(3,6-dimethyl-2-(4-(2-morpholinoethoxy)phenyl)-4-oxo-4H-chromen-8-yl)ethyl)amino)benzoic acid

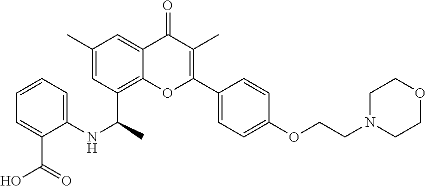 [SMILES: Cc1cc([C@@H](C)Nc2ccccc2C(=O)O)c2oc(-c3ccc(OCCN4CCOCC4)cc3)c(C)c(=O)c2c1]

(R)-2-((1-(3,6-dimethyl-2-(4-(2-morpholinoethoxy)phenyl)-4-oxo-4H-chromen-8-yl)ethyl)amino)benzoic acid is prepared in accordance with the method of Example 133.

¹H N/M (400 MHz, DMSO-d₆) δ ppm 8.37 (br d, J=5.50 Hz, 1H) 8.14 (s, 1H) 7.81 (d, J=7.88 Hz, 1H) 7.72-7.79 (n, 3H) 7.51 (d, J=1.38 Hz, 1H) 7.21 (t, J=7.75 Hz, 1H) 7.14 (d, J=8.631 Hz, 2H) 6.55 (t, J=7.50 Hz, 1H) 6.45 (d, J=8.50 Hz, 1H) 5.12 (br t, J=6.32 Hz, 1H) 4.19 (t, J=5.57 Hz, 2H) 3.55-3.62 (m, 4H) 2.73 (t, J=5.57 Hz, 2H) 2.45-2.50 (m, 4H) 2.36 (s, 3H) 2.10 (s, 3H) 1.58 (d, J=6.63 Hz, 3H))

MS m/z (ESI): 543[M+H]⁺.

### Example 142

### (R)-2-((1-(3,6-dimethyl-2-(4-(2-morpholinoethyl)phenyl)-4-oxo-4H-chromen-8-yl)ethyl)amino)benzoic acid

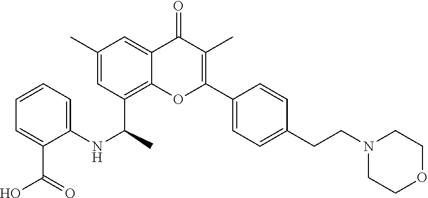 [SMILES: Cc1cc([C@@H](C)Nc2ccccc2C(=O)O)c2oc(-c3ccc(CCN4CCOCC4)cc3)c(C)c(=O)c2c1]

(R)-2-((1-(3,6-dimethyl-2-(4-(2-morpholinoethyl)phenyl)-4-oxo-4H-chromen-8-yl)ethyl)amino)benzoic acid is prepared in accordance with the method of Example 133.

¹H NMR (400 MHz, DMSO-d) δ ppm 8.52 (br s, 1H) 8.17 (s, 1H) 7.80 (dd, J=7.88, 1.38 Hz, 1H) 7.76 (s, 1H) 7.72 (d, J=8.13 Hz, 2H) 7.51 (d, J=1.88 Hz, 1H) 7.45 (d, J=8.25 Hz, 2H) 7.16 (br t, J=7.13 Hz, 1H) 6.52 (t, J=7.63 Hz, 1H) 6.40 (d, J=8.50 Hz, 1H) 5.04-5.16 (m, 1H) 3.57-3.60 (m, 4H) 2.82-2.87 (m, 2H) 2.55-2.60 (m, 2H) 2.45 (br s, 4H) 2.35 (s, 3H) 2.09 (s, 3H) 1.57 (d, J=6.63 Hz, 3H) MS ml/z (ESI): 527 [M+H]⁺.

### Example 143

### 2-((1-(2-(4-(1H-1-imidazol-1-yl)phenyl)-6-methyl-4-oxo-4H-chromen-8-yl)ethyl)amino)benzoic acid

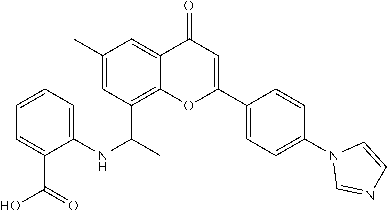 [SMILES: Cc1cc(C(C)Nc2ccccc2C(=O)O)c2oc(-c3ccc(-n4ccnc4)cc3)cc(=O)c2c1]

2-((1-(2-(4-(1H-imidazol-1-yl)phenyl)-6-methyl-4-oxo-4H-chromen-8-yl)ethyl)amino)benzoic acid is prepared in accordance with the method of Example 1.

¹H NMR (400 MHz, DMSO-d6) δ ppm 8.48-8.57 (m. 1H) 8.42-8.47 (m, 1H) 8.24-8.33 (m, 2H) 7.86-7.96 (m, 3H) 7.80-7.86 (in, H) 7.73-7.79 (in, H) 7.52-7.61 (m, 1H) 7.21-7.28 (m, 114) 7.13-7.19 (n, 2H) 6.50-6.61 (m, 2H) 5.28-5.41 (m, 1H) 2.34-2.41 (m, 3H) 1.64-1.79 (m, 3H).

MS m/z (ESI): 466 [M+H]⁺.

### Example 144

### 2-((1-(6-methyl-2-(4-(1-methyl-1H-imidazol-4-yl)phenyl)-4-oxo-4H-chromen-8-yl)ethyl)amino)benzoic acid

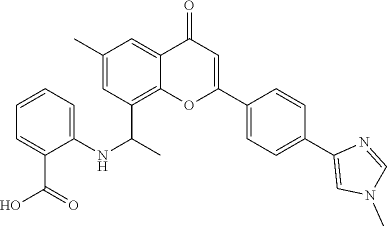 [SMILES: Cc1cc(C(C)Nc2ccccc2C(=O)O)c2oc(-c3ccc(-c4cn(C)cn4)cc3)cc(=O)c2c1]

2-((1-(6-methyl-2-(4-(n-methyl-1H-imidazol-4-yl)phenyl)-4-oxo-4H-chromen-8-yl)ethyl)amino)benzoic acid is prepared in accordance with the method of Example 1.

¹H NMR (400 MHz, DMSO-d₆) δ ppm 8.45 (br d, J=6.00 Hz, 1H) 8.12 (d, J=8.50 Hz, 2H) 7.93 (d, J=8.38 Hz, 2H) 7.79-7.85 (m, 2H) 7.70-7.76 (m, 2H) 7.56 (d, J=2.00 Hz, 1H) 7.21-7.27 (n, 1H) 7.06 (s, 1H) 6.53-6.59 (m, 2H) 5.35 (br t, J=6.38 Hz, 1H) 3.72 (s, 3H) 2.37 (s, 3H) 1.70 (d, J=6.63 Hz, 3H).

The stereoisomers of example 144: (R)-2-((1-(6-methyl-2-(4-(1-methyl-1H-imidazol-4-yl)phenyl)-4-oxo-4H-chromen-8-yl)ethyl)amino)benzoic acid and (S)-2-((1-(6-methyl-2-(4-(1-methyl-1H-imidazol-4-yl)phenyl)-4-oxo-4H-chromen-8-yl)ethyl)amino)benzoic acid

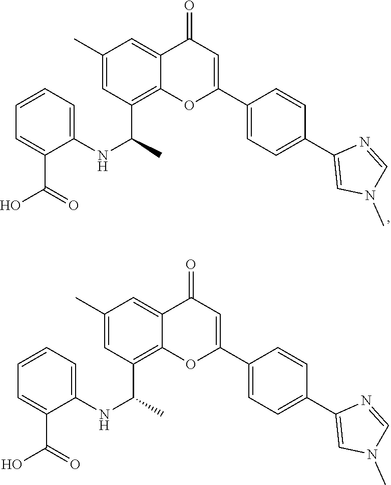 [SMILES: Cc1cc([C@@H](C)Nc2ccccc2C(=O)O)c2oc(-c3ccc(-c4cn(C)cn4)cc3)cc(=O)c2c1 ; Cc1cc([C@H](C)Nc2ccccc2C(=O)O)c2oc(-c3ccc(-c4cn(C)cn4)cc3)cc(=O)c2c1]

The stereoisomers of example 144 were prepared in accordance with the method of Example 20 by chiral SFC (column: ChiralpakIB N-3, 250×30 mm I.D., 5 μm; mobile phase: A for CO₂ and B for ETOH (0.1% NH₃·H₂O); B %: 55%-55%, min; Flow rate: 80 mL/min; Back pressure: 100 bar; Column temperature: 40° C.; Wavelength: 220 nm) to give Peak 1 (Rt=0.63 min, 144A) and Peak 2 (Rt=1.30 min, 144B). 144A and 144B were assigned arbitrarily.

Peak 1: MS m/z (ESI). 480[M+H]⁺.

¹H NMR (400 MHz, DMSO-d₆) δ ppm 9.15-9.44 (n, 1H) 8.04 (br d, J=8.50 Hz, 2H) 7.87 (br d, J=8.50 Hz, 2H) 7.79 (br d, J=7.25 Hz, 1H) 7.73 (s, 1H) 7.65 (br d, J=8.00 Hz, 2H) 7.49 (s, 1 H) 6.98 (s, 2H) 6.37-6.44 (m, 1H) 6.32 (br d, J=8.13 Hz, 1H) 5.24 (br d, J=2.63 Hz, 1H) 3.65 (s, 3H) 2.28 (s, 3H) 1.58 (br d, J=6.63 Hz, 3H)

Peak 2: MS m/z (ESI): 480[M+H]⁺.

¹H NMR (400 MHz, DMSO-d₆) δ ppm 9.11-9.30 (m, 1H) 8.04 (d, J=8.51 Hz, 2H) 7.86 (d, J=8.50 Hz, 2H) 7.80 (br d, J=7.63 Hz, 1H) 7.73 (s, 1H) 7.62-7.68 (m, 2H) 7.48 (d, J=1.75 Hz, 1H) 6.95-7.05 (m, 2H) 6.41 (t, J=7.3-8 Hz, 1H) 6.33 (br d, J=8.25 Hz, 1H) 5.24 (br s, 1H) 3.64 (s, 3H) 2.28 (s, 3H) 1.59 (d, J=6.63 Hz, 3H)

### Example 145

### 2-((1-(2-(4-(1-acetylazetidin-3-yl)phenyl)-6-methyl-4-oxo-411-chromen-8-yl)ethyl)amino)benzoic acid

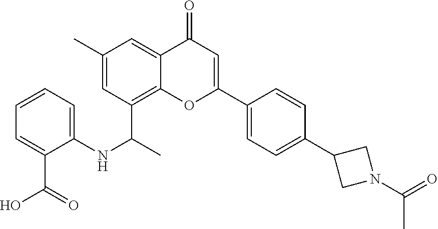 [SMILES: CC(=O)N1CC(c2ccc(-c3cc(=O)c4cc(C)cc(C(C)Nc5ccccc5C(=O)O)c4o3)cc2)C1]

2-((1-(2-(4-(1-acetylazetidin-3-yl)phenyl)-6-methyl-4-oxo-4H-chromen-8-yl)ethyl)amino)benzoic acid is prepared in accordance with the method of Example 1.

¹H NMR (400 MHz, DMSO-d6) δ ppm 8.48 (br s, 1H) 8.11 (br d, J=8.13 Hz, 2H) 7.81 (br d, J=7.13 Hz, 1H) 7.74 (s, 1H) 7.57 (br d, J=8.50 Hz, 3H) 7.21 (br t, J=7.69 Hz, 1H) 7.06 (s, 1H) 6.42-6.61 (m, 2H) 5.31 (br d, J=5.75 Hz, 1H) 4.54 (br t, J=8.50 Hz, 1H) 4.27 (br t, J=8.94 Hz, 1H) 4.17 (br t, J=6.32 Hz, 1H) 3.92 (br d, J=6.13 Hz, 1H) 3.84 (br s, 1H) 2.31-2.39 (m, 3H) 1.81 (s, 3H) 1.66 (br d, J=6.50 Hz, 3H)

MS m/z (ESI): 497 [M+H]⁺.

### Example 146

### (R)-2-((1-(2-(4-((1H-imidazol-1-yl)methyl)phenyl)-3,6-dimethyl-4-oxo-4H-chromen-8-yl)ethyl)amino)benzoic acid

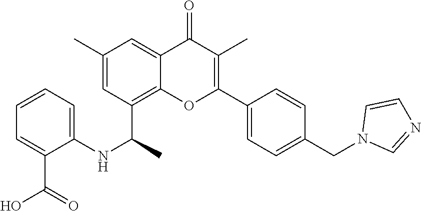 [SMILES: Cc1cc([C@@H](C)Nc2ccccc2C(=O)O)c2oc(-c3ccc(Cn4ccnc4)cc3)c(C)c(=O)c2c1]

(R)-2-((1-(2-(4-((1H-imidazol-1-yl)methyl)phenyl)-3,6-dimethyl-4-oxo-4H-chromen-8-yl)ethyl)amino)benzoic acid is prepared in accordance with the method of Example 133.

¹H NMR (400 MHz, METHANOL-d4) δ ppm 7.98 (br s, 1H) 7.86-7.93 (m, 2H) 7.78 (d, J=8.25 Hz, 2H) 7.60 (d, J=1.88 Hz, 1H) 7.47 (d, J=8.25 Hz, 2H) 7.33-7.38 (m, 1H) 7.26-7.31 (m, 1H) 7.10-7.18 (m, 2H) 6.55 (t, J=7.69 Hz, 1H) 6.42 (d, J=8.50 Hz, 1H) 5.40 (s, 2H) 5.18 (q, J=6.59 Hz, 1H) 2.41 (s, 3H) 2.17 (s, 3H) 1.64 (d, J=6.63 Hz, 3H) MS m/z (ESI): 494 [M+H]⁺.

### Example 147

### (R)-2-((1-(3,6-dimethyl-2-(4-((4-methylpiperazin-1-yl)methyl)phenyl)-4-oxo-411-chromen-8-yl)ethyl)amino)benzoic acid

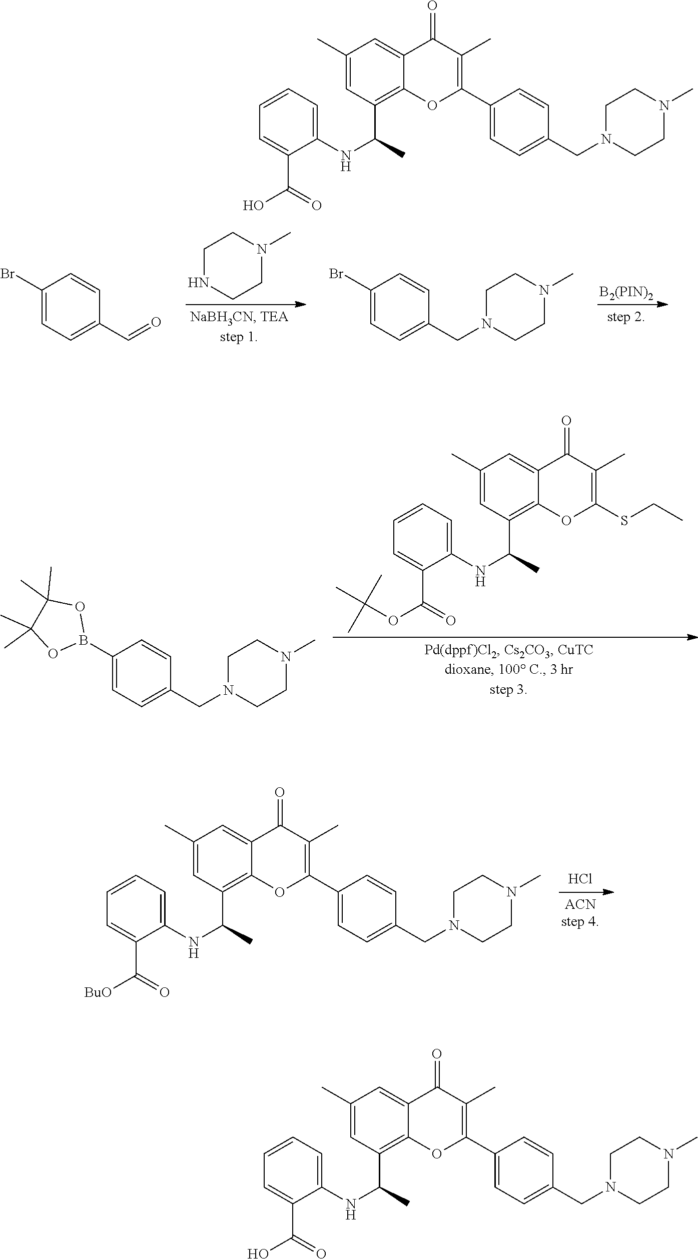

### Step 1

To a solution of 4-bromobenzaldehyde (2.77 g, 14.98 mmol, 1.5 eq), 1-methylpiperazine (1 g, 9.98 mmol, 1.11 mL, 1 eq) and NaBH(OAc)₃ (3.17 g, 14.98 mmol, 1.5 eq) in DCM (10 mL) was added CH₃COOH (599.5 mg, 9.98 mmol, 571.00 uL, 1 eq) at 25° C. The reaction mixture was stirred at 25° C. for 1 hr. The reaction mixture was extracted with ethyl acetate (30 ml*3). The combined organic layers were dried over anhydrous Na₂SO₄, filtered and concentrated under reduced pressure to give a crude product. The crude product was without purification. Compound 1-[(4-bromophenyl) methyl]-4-methyl-piperazine (1 g, crude) was obtained as a yellow oil.

MS m/z (ESI): 271 [M+H]⁺.

### Step 2

To a solution of 1-[(4-bromophenyl)methyl]-4-methyl-piperazine (700 mg, 2.60 mmol, 1 eq), 4,4,5,5-tetramethyl-2-(4,4,5,5-tetramethyl-1,3,2-dioxaborolan-2-yl)-1,3,2-dioxaborolane (1.32 g, 5.20 mmol, 2 eq) and KOAc (765 mg, 7.80 mmol, 3 eq) in dioxane (20 mL) was added Pd(dppf)Cl₂·CH₂Cl₂ (212 mg, 260.05 μmol, 0.1 eq). The mixture was stirred at 80° C. for 2 hr under N₂ atmosphere. The reaction mixture was diluted with EtOAc (10 mL) and filtered to remove the insoluble and concentrated under vacuum to give the crude product. The residue was purified by prep-HPLC (column: Xtimate C18 150*40 mm*10 um; mobile phase: [water (FA)-ACN]; B %: −0%-28%, 36 min). Compound 1-methyl-4-[[4-(4,4,5,5-tetramethyl-1,3,2-dioxaborolan-2-yl)phenyl]methyl]piperazine (105 mg, 179.42 μmol, 6% yield, 54% purity) was obtained as a white solid.

MS m/z (ESI): 317[M+H]⁺.

### Step 3

A mixture of 1-methyl-4-[[4-(4,4,5,5-tetramethyl-1,3,2-dioxaborolan-2-yl) phenyl]methyl]piperazine (69.7 mg, 220.46 μmol, 2 eq), tert-butyl 2-[[(1R)-1-(2-ethylsulfanyl-3,6-dimethyl-4-oxo-chromen-8-yl)ethyl]amino]benzoate (50 mg, 110.23 μmol, 1 eq), Cs₂CO₃ (71.8 mg, 220.46 μmol, 2 eq), thiophene-2-carbonyloxycopper (42.0 mg, 220.46 μmol, 2 eq) and Pd(dppf)Cl₂·CH₂Cl₂ (18.0 mg, 22.05 μmol, 0.2 eq) in dioxane (3 mL) was degassed and purged with N₂ for 3 tires, and then the mixture was stirred at 100° C. for 12 hr under N₂ atmosphere. The reaction mixture was diluted with EtOAc (50 mL) and filtered to remove the insoluble and concentrated under vacuum to give the crude product. The crude product was directly used to the next step and no further more purification. Compound butyl2-[[(1R)-1-[3,6-dimethyl-2-[4-[(4-methylpiperazin-1-yl)methyl]phenyl]-4-oxo-chromen-8-yl]ethyl]amino]benzoate (50 mg, 85.95 μmol, 77% yield) was obtained as a gray solid.

MS m/z (ESI): 582 [M+H]⁺.

### Step 4

To a solution of tert-butyl 2-[[(1R)-1-[3,6-dimethyl-2-[4-[(4-methylpiperazin-1-yl) methyl]phenyl]-4-oxo-chromen-8-yl]ethyl]amino]benzoate (50 mg, 85.95 μmol, 7.16 mL, 1 eq) in ACN (3 mL) was added HCl (12 M, 28.65 uL, 4 eq). The mixture was stirred at 80° C. for 2 hr. The reaction mixture was filtered to remove the insoluble and concentrated under vacuum to give the crude product. The residue was purified by prep-HPLC (column: Welch Xtimate C18 150*30 mm*5 um; mobile phase: [water (FA)-ACN]; B %: 0%-40%, 30 min). Compound 2-[[(1R)-1-[3,6-dimethyl-2-[4-[(4-methylpiperazin-1-yl)methyl]phenyl]-4-oxo-chromen-8-yl]ethyl]amino]benzoic acid (10.1 mg, 18.62 μmol, 21% yield, 96% purity) was obtained as a gray solid.

¹H NMR (400 MHz, DMSO-d₆) δ ppm 8.47 (br s, 1H) 7.77-7.82 (m, 2H) 7.77 (s, 1H) 7.75 (s, 1H) 7.55 (d, J=2.13 Hz, 1H) 7.51 (s, 1H) 7.49 (s, 1H) 7.14-7.20 (m, 1H) 6.52 (t, J=7.50 Hz, 1H) 6.44 (d, J=8.25 Hz, 1H) 5.10 (br t, J=5.94 Hz, 1H) 3.59 (s, 2H) 2.31-2.48 (m, 11H) 2.20 (s, 3H) 2.10 (s, 3H) 1.60 (d, J=6.63 Hz, 3H) MS m/z (ESI): 526 [M+H]⁺.

### Example 148

### (R)-2-((1-(3,6-dimethyl-2-(4-((4-methyl-3-oxopiperazin-1-yl)methyl)phenyl)-4-oxo-4H-chromen-8-yl)ethyl)amino)benzoic acid

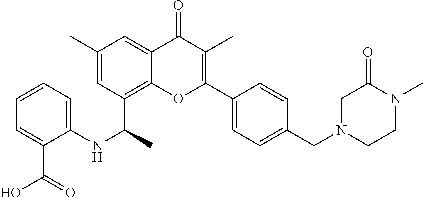 [SMILES: Cc1cc([C@@H](C)Nc2ccccc2C(=O)O)c2oc(-c3ccc(CN4CCN(C)C(=O)C4)cc3)c(C)c(=O)c2c1]

(R)-2-((1-(3, 6-dimethyl-2-(4-((4-methyl-3-oxopiperazin-1-yl)methyl)phenyl)-4-oxo-4H-chromen-8-yl)ethyl)amino)benzoic acid is prepared in accordance with the method of Example 133.

¹H NMR (400 MHz, DMSO-d₆) δ ppm 8.35 (br d, J=5.88 Hz, 1H) 7.76-7.82 (m, 3H) 7.74-7.83 (m, 1H) 7.49-7.56 (m, 3H) 7.16-7.23 (m, 1H) 6.54 (t, J=7.50 Hz, 1H) 6.47 (d, J=8.50 Hz, 1H) 5.08-5.16 (m, 1H) 3.64 (s, 2H) 3.28 (br s, 2H) 3.01 (s, 2H) 2.83 (s, 3H) 2.70 (t, J=5.50 Hz, 2H) 2.37 (s, 3H) 2.09 (s, 3H) 1.60 (d, J=6.63 Hz, 3H) MS m/z (ESI): 540[M+H]⁺.

### Example 149

### (R)-2-((1-(3,6-dimethyl-2-(4-(1-methyl-1-pyrazol-4-yl)phenyl)-4-oxo-4H-chromen-8-yl)ethyl)amino)benzoic acid

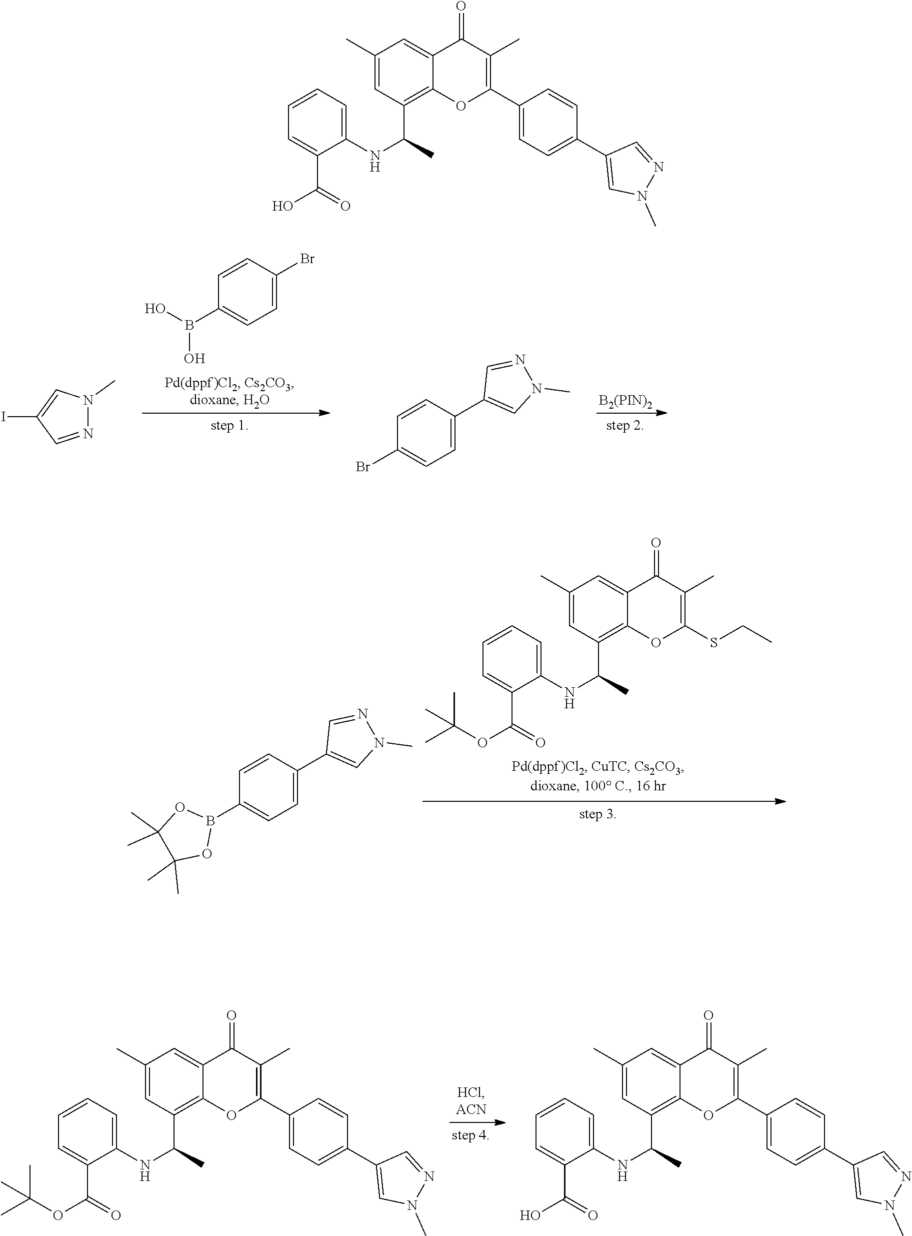

### Step 1

A mixture of 4-iodo-1-methyl-pyrazole (2 g, 9.62 mmol, 1 eq), (4-bromophenyl)boronic acid (2.32 g, 11.54 mmol, 1.2 eq), Cs₂CO₃ (9.40 g, 28.85 mmol, 3 eq), Pd(dppf)Cl₂·CH₂Cl₂ (1.57 g, 1.92 mmol, 0.2 eq) in dioxane (20 mL) and H₂O (5 mL) was degassed and purged with N₂ for 3 times, and then the mixture was stirred at 100° C. for 16 hr under N₂ atmosphere. LCMS showed one main peak desired mass was detected. The reaction mixture was filtered to remove the insoluble and concentrated under vacuum to give the crude product. The residue was purified by flash silica gel chromatography (ISCO®; 20 g SepaFlash® Silica Flash Column, Fluent of 0˜20% Ethyl acetate/Petroleum ether gradient @ 40 mL/min). Compound 4-(4-bromophenyl)-1-methyl-pyrazole (630 mg, 2.66 mmol, 27% yield) was obtained as a yellow solid. MS m/z (ESI): 239[M+H]⁺.

### Step 2

A mixture of 4-(4-bromophenyl)-1-methyl-pyrazole (630 mg, 2.66 mmol, 1 eq), 4,4,5,5-tetramethyl-2-(4,4,5,5-tetramethyl-1,3,2-dioxaborolan-2-yl)-1,3,2-dioxaborolane (1.35 g, 5.31 mmol, 2 eq), KOAc (782 mg, 7.97 mmol, 3 eq), Pd(dppf)Cl₂·CH₂Cl₂ (217 mg, 265.72 umol, 0.1 eq) in dioxane (8 mL) was degassed and purged with N₂ for 3 times, and then the mixture was stirred at 80° C. for 16 hr under N₂ atmosphere. LCMS showed one main peak desired mass was detected. The reaction mixture was diluted with H₂O (50 mL) and extracted with ethyl acetate (50 mL*3). The combined organic layers were dried over [Na₂SO₄], filtered and concentrated under reduced pressure to give a crude product. The residue was purified by flash silica gel chromatography (ISCO®; 12 g SepaFlash® Silica Flash Column, Eluent of 0˜25% Ethyl acetate/Petroleum ether gradient @ 40 mL/min). Compound 1-methyl-4-[4-(4,4,5,5-tetramethyl-1,3,2-dioxaborolan-2-yl)phenyl]pyrazole (740 mg, 2.60 mmol, 98% yield) was obtained as a white solid.

MS m/z (ESI): 285[M+H]⁺.

### Step 3

A mixture of tert-butyl 2-[[(1R)-1-(2-ethylsulfanyl-3,6-dimethyl-4-oxo-chromen-8-yl) ethyl]amino]benzoate (100 mg, 220.46 umol, 1 eq), i-methyl-4-[4-(4,4,5,5-tetramethyl-1,3,2-dioxaborolan-2-yl)phenyl]pyrazole (157 mg, 551.16 umol, 2.5 eq), Pd(dppf) Cl₂·CH₂Cl₂ (36 mg, 44.09 umol, 0.2 eq), Cs₂CO₃ (144 mg, 440.92 umol, 2 eq) and thiophene-2-carbonyloxycopper (84 mg, 440.92 umol, 2 eq) in dioxane (3 mL) was degassed and purged with N₂ for 3 times, and then the mixture was stirred at 100° C. for 16 hr under N₂ atmosphere. LC MS showed one main peak desired mass was detected. The reaction mixture was filtered to remove the insoluble and concentrated under vacuum to give the crude product. The crude product was purified by flash silica gel chromatography (ISCO®; 12 g SepaFlash® Silica Flash Column, Eluent of 0-50% Ethyl acetate/Petroleum ether gradient @) 15 mL/min). Compound tert-butyl 2-[[(1R)-1-[3,6-dimethyl-2-[4-(1-methyl pyrazol-4-yl)phenyl]-4-oxo-chromen-8-yl]ethyl]amino]benzoate (84 mg, 144.90 umol, 65% yield, 94% purity) was obtained as a pink solid.

MS m/z (ESI): 550[M+H]⁺.

### Step 4

To a solution of tert-butyl 2-[[(1R)-1-[3,6-dimethyl-2-[4-(1-methylpyrazol-4-yl) phenyl]-4-oxo-chromen-8-yl]ethyl]amino]benzoate (84 mg, 152.82 umol, 1 eq) in ACN (1 mL) was added HCl (12 M, 12.74 uL, 1 eq). The mixture was stirred at 80° C. for 16 hr. LCMS showed one main peak desired mass was detected. The reaction mixture was filtered. The filtrate was purified by prep-HPLC (column: Welch Xtimate C18 150*30 mm*5 um; mobile phase: [water (FA)-ACN]; B %: 34%-74%, 25 min). Compound 2-[[(1R)-1-[3,6-dimethyl-2-[4-(1-methylpyrazol-4-yl)phenyl]-4-oxo-chromen-8-yl]ethyl]amino]benzoic acid (26 mg, 52.56 umol, 34% yield, 99% purity) was obtained as a white solid.

¹H NMR (400 MI-z, DMSO-d₆) δ ppm 8.36 (br s, 1H) 8.23 (s, 1H) 7.96 (s, 1H) 7.71-7.81 (m, 6H) 7.51 (br s, 1H) 7.19 (br t, J=7.44 Hz, 1H) 6.54 (br t, J=7.38 Hz, 1H) 6.45 (br d, J=8.38 Hz, 1H) 5.13 (br s, 1H) 3.88 (s, 3H) 2.34 (s, 3H) 2.10 (s, 3H) 1.58 (br d, J=6.38 Hz, 3H). MS m/z (ESI) 494 [M+H]⁺.

### Example 150

### (R)-2-((1-(3,6-dimethyl-2-(6-(1-methyl-H-pyrazol-4-yl)pyridin-3-yl)-4-oxo-41-chromen-8-yl)ethyl)amino)benzoic acid

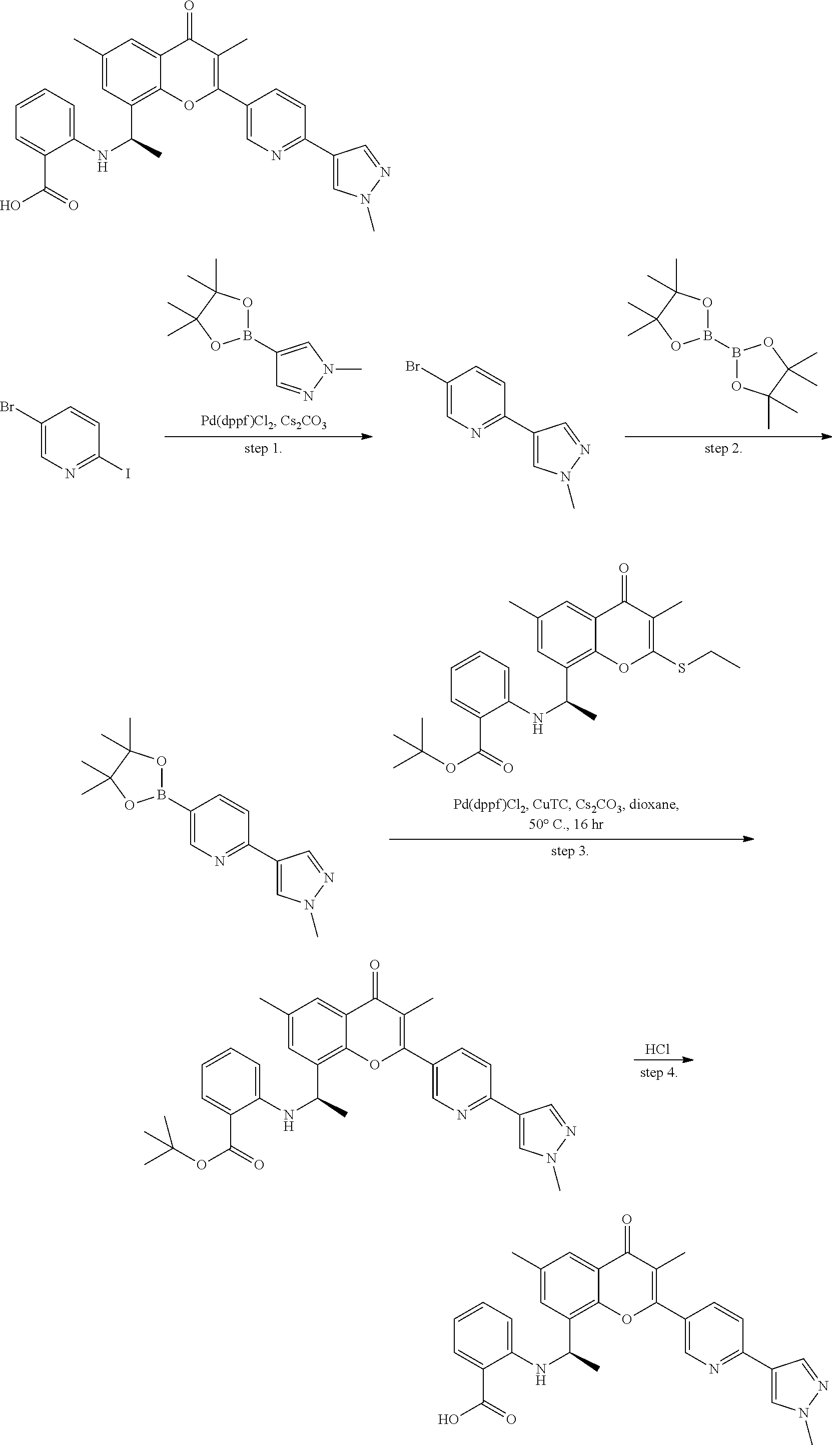

### Step 1

To a solution of 5-bromo-2-iodo-pyridine (1 g, 3.52 mmol, 1 eq), I-methyl-4-(4,4,5,5-tetramethyl-1,3,2-dioxaborolan-2-yl)pyrazole (733 mg, 3.52 mmol, 1 eq), Cs₂CO₃ (2 M, 3.52 mL, 2 eq) and cyclopentyl (diphenyl) phosphane; dichloromethane; dichloropalladium; iron (575 mg, 704.49 μmol, 0.2 eq) were added in toluene (15 mL) and H₂O (5 mL). Then the mixture was stirred at 100° C. under N₂ for 16 h. LCMS showed one main peak with desired mass was detected. The reaction mixture was filtered to remove the insoluble and concentrated under vacuum to give the crude product. The crude product was purified by flash silica gel chromatography (ISCO®; 20 g SepaFlash® Silica Flash Column, Eluent of 0-50% Ethyl acetate/Petroleum ether gradient @ 30 mL/min). Compound 5-bromo-2-(1-methylpyrazol-4-yl)pyridine (390 mg, 1.48 mmol, 42% yield, 90% purity) was obtained as a white solid.

MS m/z (ESI): 238[M+H]⁺.

### Step 2

To a solution of 5-bromo-2-(n-methylpyrazol-4-yl) pyridine (300 mg, 1.26 mmol, 1 eq), 4,4,5,5-tetramethyl-2-(4,4,5,5-tetramethyl-1,3,2-dioxaborolan-2-yl)-1,3,2-dioxaborolane (640 mg, 2.52 mmol, 2 eq), KOAc (371.00 mg, 3.78 mmol, 3 eq), Pd₂(dba)₃ (115.39 mg, 126.01 μmol, 0.1 eq) and ditert-butyl-[2-(2,4,6-triisopropylphenyl)phenyl]phosphane (54 mg, 126.01 μmol, 0.1 eq) were added in dioxane (8 mL). Then the mixture was stirred at 100° C. under N₂ for 3b. LCMS showed one main peak with desired mass was detected. The reaction mixture was concentrated under vacuum to give the crude product. The crude product was purified by flash silica gel chromatography (ISCO®; 20 g SepaFlash® Silica Flash Column, Eluent of 0˜80% Ethyl acetate/Petroleum ether gradient @ 35 mL/min). Compound 2-(1-methylpyrazol-4-yl)-5-(4,4,5,5-tetramethyl-1,3,2-dioxaborolan-2-yl)pyridine (160 mg, 276.90 μmol, 22% yield, 49% purity) was obtained as a yellow oil.

MS m/z (ESI): 204[M+H]⁺.

### Step 3

To a solution of tert-butyl 2-[[(1R)-1-(2-ethylsulfanyl-3,6-dimethyl-4-oxo-chromen-8-yl)ethyl]amino]benzoate (50 mg, 110.23 μmol, 1 eq), 2-(1-methylpyrazol-4-yl)-5-(4,4,5,5-tetramethyl-1,3,2-dioxaborolan-2-yl)pyridine (63 mg, 22046 μmol, 2 eq), cyclopentyl(diphenyl)phosphane; dichloromethane; dichloropalladium; iron (36 mg, 44.09 μmol, 0.4 eq), thiophene-2-carbonyloxycopper (84 mg, 440.92 μmol, 4 eq) and Cs₂CO₃ (72 mg, 220.46 μmol, 2 eq) were added in dioxane (10 mL). Then the mixture was stirred at 50° C. under N₂ for 16 h. LC MS showed one main peak with desired mass was detected. The reaction mixture was concentrated under vacuum to give the crude product. The crude product no further more purification, it was directly used to the next step. Compound tert-butyl 2-[[(1R)-1-[3,6-dimethyl-2-[6-(1-methylpyrazol-4-yl)-3-pyridyl]-4-oxo-chromen-8-yl]ethyl] amino]benzoate (200 mg, 101.41 μmol, 92.00% yield, 27.920% purity) was obtained as a brown oil.

MS m/z (ESI): 551[M+H]⁺.

### Step 4

To a solution of tert-butyl 2-[[(1R)-1-[3,6-dimethyl-2-[6-(1-methylpyrazol-4-yl)-3-pyridyl]-4-oxo-chromen-8-yl]ethyl]amino]benzoate (160 mg, 81.13 μmol, 27% purity, 1 eq) and HCl (12 M, 13.52 uL, 2 eq) were added in MeCN (10 mL), then the mixture was stirred at 80° C. for 2 h. LCMS showed one main peak with desired mass was detected. The reaction mixture was concentrated under vacuum to give the crude product. The crude product was purified by prep-HPLC (column: Phenomenex C18 75*30 mm*3 um; mobile phase: [water (TFA)-ACN]; B %: 12%-52%, 36 min). Compound 2-[[(1R)—1-[3,6-dimethyl-2-[6-(1-methylpyrazol-4-yl)-3-pyridyl]-4-oxo-chromen-8-yl]ethyl]amino]benzoic acid (21.5 mg, 43.43 μmol, 53% yield, 99.9% purity) was obtained as a yellow solid.

¹H NMR (400 MHz, DMSO-d₆) δ ppm 8.87-9.01 (m, 1H) 8.40-8.47 (m, 1H) 8.34-8.39 (m, 1H) 8.17-8.23 (m, 1H) 8.09-8.14 (m, 1H) 7.84-7.90 (m, 1H) 7.76-7.83 (m, 2H) 7.50-7.56 (m, 1H) 7.16-7.26 (m, 1H) 6.51-6.58 (m, 1H) 6.44-6.49 (m, 1H) 5.13-5.22 (m, 1H) 3.88-398 (m, 3H) 2.34-2.40 (m, 3H) 210-2.17 (m, 3H) 1.55-1.66 (m, 3H)

MS m/z (ESI): 495 [M+H]⁺

### Example 151

### (R)-2-((1-(3,6-dimethyl-2-(4-((1-methyl-1H-pyrazol-4-yl)methyl)phenyl)-4-oxo-4H-chromen-8-yl)ethyl)amino)benzoic

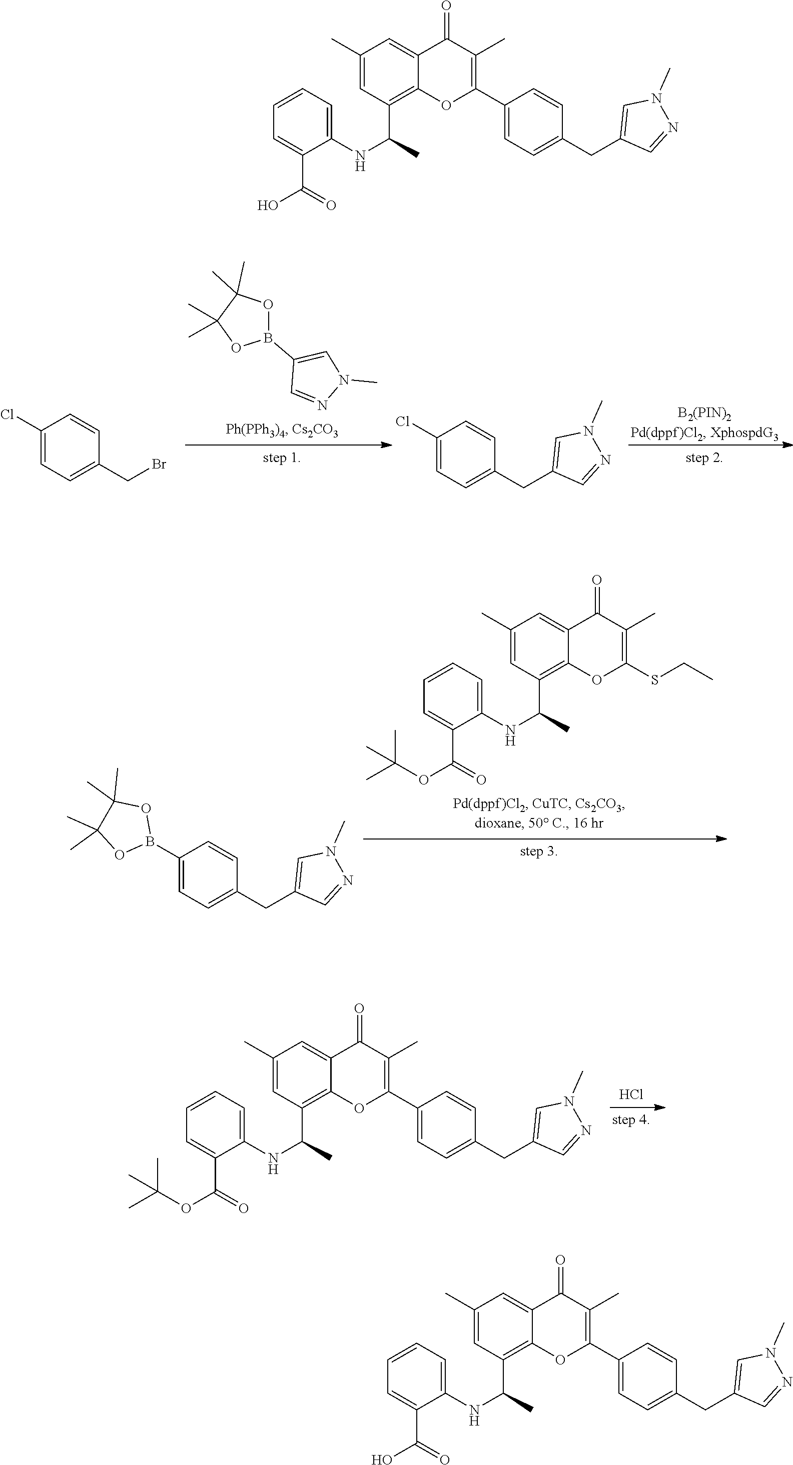

### Step 1

To a solution of 1-(bromoethyl)-4-chloro-benzene (1 g, 4.87 mmol, 1 eq), 1-methyl-4-(4,4,5,5-tetramethyl-1,3,2-dioxaborolan-2-yl)pyrazole (1 g, 4.87 mmol, 1 eq), cyclopentyl(diphenyl)phosphane; dichloromethane; dichloropalladium; iron (795 mg, 973.33 μmol, 0.2 eq) and Cs₂CO₃ (2 M, 4.87 mL, 2 eq) were added in H₂O (5 mL) and dioxane (20 mL). Then the mixture was stirred at 80° C. under N₂ for 16 hours. LCMS showed one main peak with desired mass was detected. The reaction mixture was filtered to remove the insoluble and concentrated under vacuum to give the crude product. The crude product was purified by flash silica gel chromatography (ISCO®; 40 g SepaFlash® Silica Flash Column, Eluent of 0˜40% Ethyl acetate/Petroleum ether gradient @ 50 mL/min). Compound 4-[(4-chlorophenyl)methyl]-1-methyl-pyrazole (600 mg, 2.32 mmol, 47% yield, 80% purity) was obtained as a yellow oil.

MS m/z (ESI): 207 [M+H]⁺.

### Step 2

To a solution of 4-[(4-chlorophenyl)methyl]-1-methyl-pyrazole (250 mg, 1.21 mmol, 1 eq), 4,4,5,5-tetramethyl-2-(4,4,5,5-tetramethyl-1,3,2-dioxaborolan-2-yl)-1,3,2-dioxaborolane (461 mg, 1.81 mmol, 1.5 eq), KOAc (356 mg, 3.63 mmol, 3 eq) and [2-(2-aminophenyl)phenyl]palladium(1+); dicyclohexyl-[2-(2,4,6-triisopropylphenyl)phenyl]phosphane; methanesulfonate (205 mg, 241.93 μmol, 0.2 eq) in dioxane (10 mL) was stirred at 100° C. under N₂ for 16 h. LCMS showed one main peak with desired mass was detected. The reaction mixture was concentrated under vacuum to give the crude product. The crude product was purified by flash silica gel chromatography (ISCO®; 20 g SepaFlash® Silica Flash Column, Eluent of 0-50% Ethyl acetate/Petroleum ether gradient @. 35 mL/min). Compound 1-methyl-4-[[4-(4,4,5,5-tetramethyl-1,3,2-dioxaborolan-2-yl)phenyl]methyl] pyrazole (550 mg, crude) was obtained as a yellow oil.

MS m/z (ESI): 299 [M+H]⁺.

### Step 3

To a solution of tert-butyl 2-[[(1R)-1-(2-ethylsulfanyl-3,6-dimethyl-4-oxo-chromen-8-yl) ethyl]amino]benzoate (50 mg, 110.23 umol, 1 eq), 1-methyl-4-[[4-(4,4,5,5-tetramethyl-1,3,2-dioxaborolan-2-yl)phenyl]methyl]pyrazole (132 mg, 440.92 μmol, 4 eq), cyclopentyl(diphenyl)phosphane; dichloromethane; dichloropalladium; iron (36 mg, 44.09 μmol, 0.4 eq), thiophene-2-carbonyloxycopper (84 mg, 440.92 μmol, 4 eq) and Cs₂CO₃ (108 mg, 330.69 μmol, 3 eq) were added in dioxane (10 mL). Then the mixture was stirred at 50° C. under N₂ for 16 h. LCMS showed one main peak with desired mass was detected. The reaction mixture was concentrated under vacuum to give the crude product. The crude product was purified by flash silica gel chromatography (ISCO®; 12 g SepaFlash® Silica Flash Column, Fluent of 0˜55% Ethyl acetate/Petroleum ether gradient @35 mL/min). Compound tert-butyl 2-[[(1R)-1-[3,6-dimethyl-2-[4-[(1-methylpyrazol-4-yl) methyl]phenyl]-4-oxo-chromen-8-yl]ethyl]amino]benzoate (100 mg, 78.58 μmol, 71% yield, 44% purity) was obtained as a brown oil

MS m/z (ESI): 564 [M+H]⁺.

### Step 4

To a solution of tert-butyl 2-[[(1R)-1-[3, 6-dimethyl-2-[4-[(i-methylpyrazol-4-yl)methyl]phenyl]-4-oxo-chromen-8-yl]ethyl]amino]benzoate (50 mg, 39.29 μmol, 44.296% purity, 1 eq) in MeCN (2 mL) was added HCl (12 M, 3 uL, 4 eq). The mixture was stirred at 80° C. for 2 hr. LCMS showed one main peak with desired mass was detected. The reaction mixture was concentrated under vacuum to give the crude product. The crude product was purified by prep-HPLC (column: Welch Xtimate C18 150*30 mm*5 um; mobile phase: [water (FA)-ACN]; B %: 34%-74%, 25 min). Compound 2-[[(1R)-1-[3,6-dimethyl-2-[4-[(1-methylpyrazol-4-yl)methyl]phenyl]-4-oxo-chromen-8-yl]ethyl]amino]benzoic acid (2 mg, 3.93 μmol, 10% yield, 99% purity) was obtained as a white solid.

¹H NMR (400 MHz, DMSO-d₆) δ ppm 8.33-8.45 (m, 1H) 7.81 (br d, J=7.38 Hz, 1H) 7.75-7.78 (m, 1H) 7.70-7.74 (m, 2H) 7.49-7.57 (m, 2H) 7.39-7.45 (m, 2H) 7.30-7.35 (m, 1H) 7.15-7.23 (m, 1H) 6.50-6.58 (m, 1H) 6.40-6.46 (m, 1H) 5.06-5.16 (m, 1H) 3.84-3.90 (m, 2H) 3.74-3.81 (m, 3H) 232-2.40 (m, 3H) 2.04-2.13 (m, 3H) 1.53-1.62 (m, 3H) MS m/z (ESI): 508[M+H]⁺.

### Example 152

### 2-((1-(6-methyl-2-(2-morpholinopyrimidin-5-yl)-4-oxo-4H-chromen-8-yl)ethyl)amino)benzoic acid

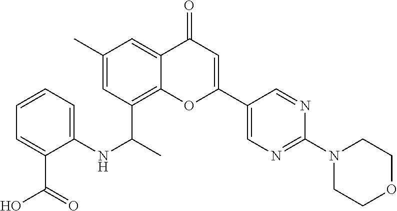 [SMILES: Cc1cc(C(C)Nc2ccccc2C(=O)O)c2oc(-c3cnc(N4CCOCC4)nc3)cc(=O)c2c1]

2-((1-(6-methyl-2-(2-morpholinopyrimidin-5-yl)-4-oxo-4H-chromen-8-yl)ethyl)amino)benzoic acid is prepared in accordance with the method of Example 1.

¹H NMR (400 MHz, DMSO-d₆) δ ppm 9.08 (s, 2H) 8.36-8.59 (m, 1H) 7.81 (d, J=8.13 Hz, 1H) 7.72 (s, 1H) 7.51 (s, 1H) 721 (br t, J=7.63 Hz, 1H) 7.00 (s, 1H) 6.42-6.62 (m, 2H) 5.32 (br t, J=5.94 Hz, 1H) 3.84-3.87 (m, 4H) 3.69 (br d, J=4.38 Hz, 4H) 2.34 (s, 3H) 1.64 (br d, J=6.50 Hz, 3H)

MS m/z (ESI): 487 [M+H]⁺.

### Example 153

### (R)-2-((1-(2-(6-(4-acetylpiperazin-1-yl)pyridin-3-yl)-3,6-dimethyl-4-oxo-4H-chromen-8-yl)ethyl)amino)benzoic acid

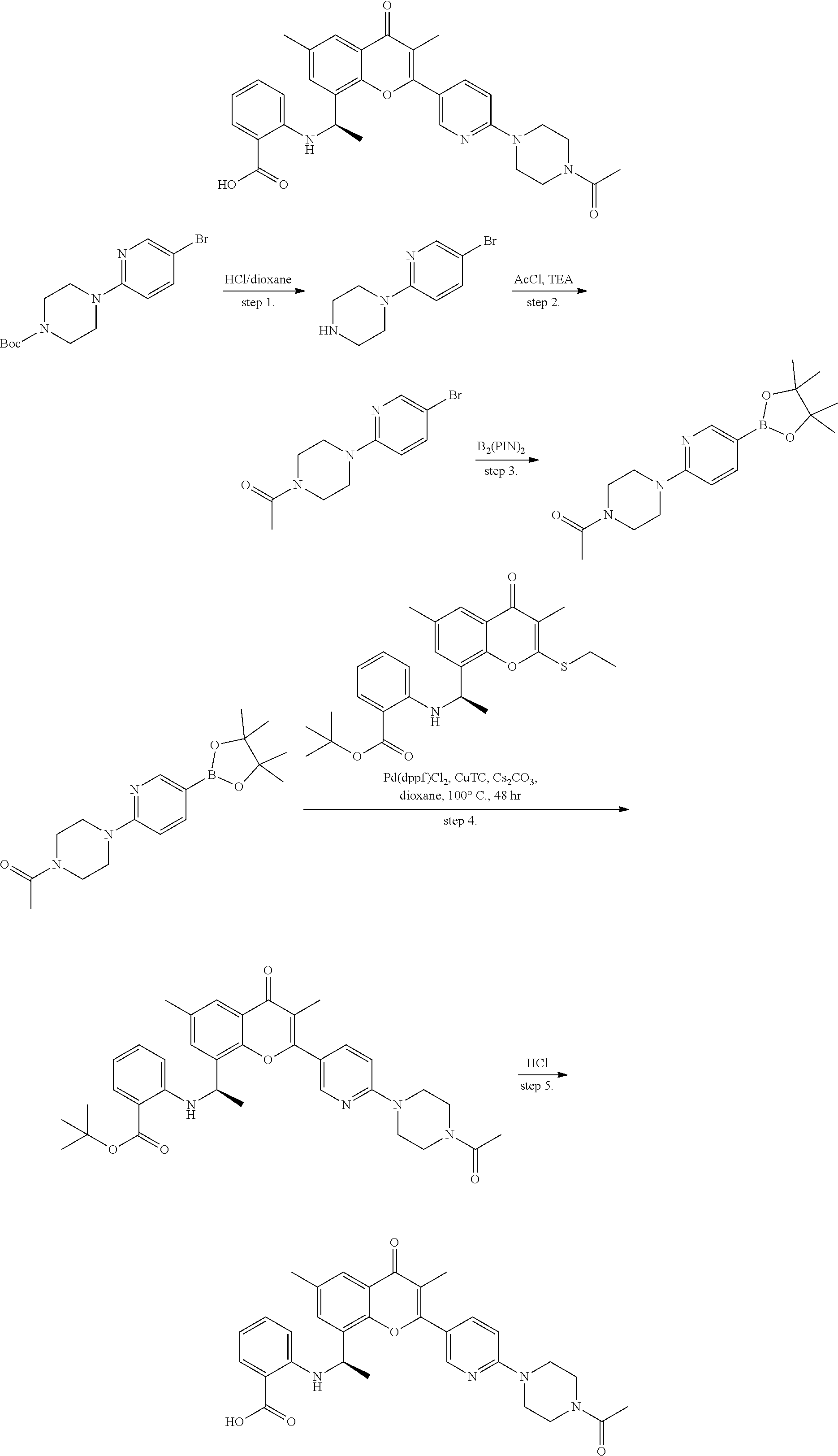

### Step 1

To a solution of tert-butyl 4-(5-bromo-2-pyridyl)piperazine-1-carboxylate (2 g, 5.84 mmol, 1 eq) in DCM (15 mL) was added HCl/dioxane (10 mL). The mixture was stirred at 25° C. for 1 hr. LCMS showed desired MS was detected. The reaction mixture was concentrated under vacuum to give the crude product. The crude product was directly used to the next step, no future more purification. Compound 1-(5-bromo-2-pyridyl) piperazine (1.2 g, 4.96 mmol, 84% yield) was obtained as a white solid.

MS m/z (ESI): 244 [M+H]⁺.

### Step 2

To a solution of 1-(5-bromo-2-pyridyl) piperazine (1 g, 3.59 mmol, 1 eq, HCl) in DCM (1 mL) was added Ac₂O (732 mg, 7.18 mmol, 672.42 uL, 2 eq) and TEA (1 g, 10.77 mmol, 1.50 mL, 3 eq). The mixture was stirred at 25° C. for 12 hr. LCMS showed desired MS was detected. The reaction mixture was added H₂O (30 ml) and extracted with ethyl acetate (30 ml*3). The combined organic layers were dried over [Na₂SO₄], filtered and concentrated under reduced pressure to give a crude product. The crude product was purified by flash silica gel chromatography (ISCO®; 12 g SepaFlash® Silica Flash Column, Eluent of 0˜80% Ethyl acetate/Petroleum ether gradient @ 40 mL/min). Compound 1-[4-(5-bromo-2-pyridyl) piperazin-1-yl] ethanone (770 mg. 2.60 mmol, 72% yield) was obtained as a white solid.

MS m/z (ESI): 286 [M+H]⁺.

### Step 3

To a solution of 1-[4-(5-bromo-2-pyridyl)piperazin-1-yl]ethanone (300 mg, 1.06 mmol, 1 eq), 4,4,5,5-tetramethyl-2-(4,4,5,5-tetramethyl-1,3,2-dioxaborolan-2-yl)-1,3,2-dioxaborolane (536 mg, 2.11 mmol, 2 eq), KOAC (310 mg, 3.17 mmol, 3 eq), [2-(2-aminophenyl)phenyl]palladium(1+); dicyclohexyl-[2-(2,4,6-triisopropylphenyl) phenyl]pbosphane; methanesulfonate (178 mg, 211.15 μmol, 0.2 eq) in dioxane (5 mL) was degassed and purged with N₂ for 3 times, and then the mixture was stirred at 100° C. for 16 hr under N₂ atmosphere. LCMS showed desired MS was detected. The reaction mixture was added H₂O (20 ml) and extracted with ethyl acetate (30 ml*3). The combined organic layers were dried over anhydrous Na₂SO₄, filtered and concentrated under reduced pressure to give a crude product. The crude product was purified by flash silica gel chromatography (ISCOR; 12 g SepaFlash® Silica Flash Column, Eluent of 0-80% Ethyl acetate/Petroleum ether gradient @ 30 mL/min). Compound 1-[4-[5-(4,4,5,5-tetramethyl-1,3,2-dioxaborolan-2-yl)-2-pyridyl]piperazin-1-yl]ethanone (275 mg, 830.27 μmol, 79% yield) was obtained as a white solid.

MS m/z (ES1): 250 [M+H]⁺.

### Step 4

To a solution of tert-butyl 2-[[(1R)-1-(2-ethyl sulfanyl-3,6-dimethyl-4-oxo-chromen-8-yl)ethyl]amino]benzoate (50 mg, 110.23 μmol, 1 eq), [6-(4-acetylpiperazin-1-yl)-3-pyridyl]boronic acid (54 mg, 220.46 μmol, 2 eq), thiophene-2-carbonyloxycopper (84 mg, 440.92 μmol, 4 eq), cyclopentyl(diphenyl) phosphane; dichloromethane; dichloropalladium; iron (36 mg, 44.09 μmol, 0.4 eq) and Cs₂CO₃ (71 mg, 220.46 μmol, 2 eq) in dioxane (3 mL) was degassed and purged with N₂ for 3 times, and then the mixture was stirred at 50° C. for 16 hr under N₂ atmosphere. LCMS showed desired MS was detected. The reaction mixture was filtered to remove the insoluble and concentrated under vacuum to give the crude product. The crude product was directly used to the next step, no future more purification. Compound tert-butyl 2-[[(1R)-1-[2-[6-(4-acetylpiperazin-1-yl)-3-pyridyl]-3, 6-dimethyl-4-oxo-chromen-8-yl] ethyl] amino] benzoate (50 mg, 83.79 μmol, 76% yield) was obtained as a black oil.

MS m/z (ESI): 597 [M+H]⁺.

### Step 5

To a solution of tert-butyl 2-[[(1R)-1-[2-[6-(4-acetylpiperazin-1-yl)-3-pyridyl]-3, 6-dimethyl-4-oxo-chromen-8-yl] ethyl] amino] benzoate (40 mg, 67.03 μmol, 1 eq) in ACN (3 mL) was added HCl (12 M, 22.34 uL, 4 eq). The mixture was stirred at 80° C. for 1 hr. LCMS showed desired MS was detected. The reaction mixture was filtered to remove the insoluble and concentrated under vacuum to give the crude product. The crude product was purified by prep-HPLC (column: Welch Xtimate C18 150*30 mm*5 um; mobile phase: [water (FA)-ACN]; 9B %: 24%-64%, 25 min). Compound 2-[[(1R)-1-[2-[6-(4-acetylpiperazin-1-yl)-3-pyridyl]-3, 6-dimethyl-4-oxo-chromen-8-yl] ethyl] amino] benzoic acid (4.3 mg, 7.84 μmol, 12% yield, 98% purity) was obtained as a white solid.

¹H NMR (400 MHz, DMSO-d₆) δ ppm 8.60 (d, J=2.38 Hz, 1H) 8.37 (br d, J=5.75 Hz, 1H) 7.98 (dd, J=8.94, 2.44 Hz, 1H) 7.81 (dd, J=7.88, 1.38 Hz, 1H) 7.71-7.76 (m, 3H) 7.57 (d, J=3.88 Hz, 1H) 7.44-7.50 (m, 2H) 7.41 (d, J=7.25 Hz, 1H) 7.18-7.24 (n, 1H) 7.00 (d, J=9.01 Hz, 1H) 6.55 (t, J=7.44 Hz, 1H) 6.46 (d, J=8.38 Hz, 1H) 5.15 (br t, J=6.07 Hz, 1H) 3.71-3.74 (m, 2H) 3.63-3.65 (m, 2H) 3.58-3.60 (n, 4H) 2.35 (s, 3H) 2.13 (s, 3H) 2.06 (s, 3H) 1.55-1.64 (m, 3H) MS m/z (ESI): 541 [M+H]⁺.

### Example 154

### (R)-2-((1-(2-(4-(4-acetylpiperazin-1-yl)-3-fluorophenyl)-3,6-dimethyl-4-oxo-41H-chromen-8-yl)ethyl)amino)benzoic acid

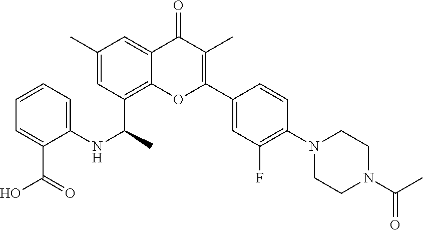 [SMILES: CC(=O)N1CCN(c2ccc(-c3oc4c([C@@H](C)Nc5ccccc5C(=O)O)cc(C)cc4c(=O)c3C)cc2F)CC1]

(R)-2-((1-(2-(4-(4-acetylpiperazin-1-yl)-3-fluorophenyl)-3,6-dimenthyl-4-oxo-4H-chromen-8-yl)ethyl)amino)benzoic acid is prepared in accordance with the method of Example 133.

¹H NMR (400 MHz, METHANOL-d₄) δ ppm 7.84-7.93 (m, 2H) 7.63 (d, J=1.75 Hz, 1H) 7.46-7.54 (m, 2H) 7.17-7.25 (m, 2H) 6.66 (br t, J=7.44 Hz, 1H) 6.47 (d, J=8.38 Hz, 1H) 5.26 (q, J=−6.30 Hz, 1H) 3.78 (dt, J=16.88, 5.13 Hz, 4H) 3.26-3.30 (m, 2H) 3.19-3.25 (m, 2H) 2.43 (s, 3H) 12-2.23 (m, 6H) 1.71 (br d, J=6.63 Hz, 3H)

MS m/z (ESI): 558 [M+H]⁺.

### Example 155

### 2-((1-(2-(6-(4-(methoxycarbonyl)piperazin-1-yl)pyridin-3-yl)-3,6-dimethyl-4-oxo-4H-chromen-8-yl)ethyl)amino)benzoic acid

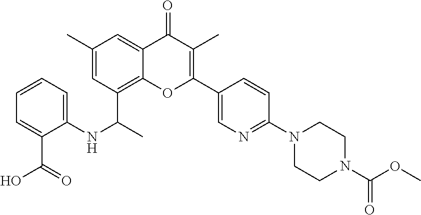 [SMILES: COC(=O)N1CCN(c2ccc(-c3oc4c(C(C)Nc5ccccc5C(=O)O)cc(C)cc4c(=O)c3C)cn2)CC1]

2-((1-(2?-(6-(4-(methoxycarbonyl)piperazin-1-yl)pyridin-3-yl)-3,6-dimethyl-4-oxo-4H-chromen-8-yl)ethyl)amino)benzoic acid is prepared in accordance with the method of Example 1.

The stereoisomers of example 155: (R)-2-((1-(2-(6-(4-(methoxycarbonyl)piperazin-1-yl)pyridin-3-yl)-3,6-dimethyl-4-oxo-4H-chromen-8-yl)ethyl)amino)benzoic acid and (S)-2-((1-(2-(6-(4-(methoxycarbonyl)piperazin-1-yl)pyridin-3-yl)-3,6-dimethyl-4-oxo-4H-chromen-8-yl)ethyl)amino)benzoic acid

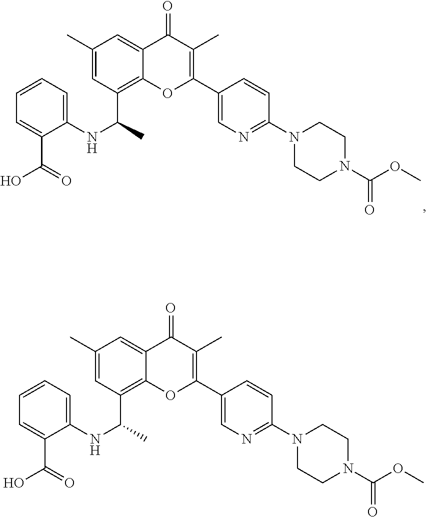 [SMILES: COC(=O)N1CCN(c2ccc(-c3oc4c([C@@H](C)Nc5ccccc5C(=O)O)cc(C)cc4c(=O)c3C)cn2)CC1 ; COC(=O)N1CCN(c2ccc(-c3oc4c([C@H](C)Nc5ccccc5C(=O)O)cc(C)cc4c(=O)c3C)cn2)CC1]

The stereoisomers of example 155 were prepared in accordance with the method of Example 20 by chiral SFC (column: ChiralpakIB N-3, 250×30 mm I.D., 5 μm; mobile phase: A for CO₂ and B for ETOH (0.1% NH₃·H₂O); B %: 40%-40%, min; Flow rate: 80 mL/min; Back pressure: 100 bar; Column temperature: 40° C.; Wavelength: 220 nm) to give Peak 1 (Rt=1.70 min) and Peak 2 (Rt=2.24 min).

Peak 1: MS m/z (ESI): 543[M+H]⁺.

¹H NMR (400 MHz, DMSO-dc) δ ppm 8.93 (d, J=2.13 Hz, 1H) 8.59 (br d, J=3.88 Hz, 1H) 8.26 (dd, J=9.07, 2.19 Hz, 1H) 7.87 (hr d, J=7.75 Hz, 1H) 7.77 (s, 1H)⁷0.57 (s, 1H) 7.26 (br t, J=7.75 Hz, 1H) 7.05 (br d, J=9.13 Hz, 1H) 6.97 (s, 1H) 6.59 (br d, J=16.38 Hz, 2H) 5.36 (br d, J=5.50 Hz, 1H) 3.76-3.77 (m, 4H) 3.75 (br s, 4H) 3.70 (br s, 3H) 2.40 (s, 3H) 1.71 (br d, J=6.50 Hz, 3H)

Peak 2: MS m/z (ESI): 543[M+H]⁺.

¹H NMR (400 MHz, DMSO-d₆) δ ppm 8.93 (d, J=2.38 Hz, 1H) 8.54 (br s, 1H) 8.26 (dd, J=9.13, 2.38 Hz, 1H) 7.88 (d, J=7.75 Hz, 1H) 7.78 (s, 1H) 7.57 (d, J=1.63 Hz, 1H) 7.27 (br t, J=7.75 Hz, 1H) 7.05 (d, J=9.26 Hz, 1H) 6.98 (s, 1H) 6.53-6.65 (m, 2H) 532-5.39 (M, 1H) 3.77 (br d, J=4.50 Hz, 4H) 3.75 (br s, 4H) 3.70 (s, 3H) 2.40 (s, 3H) 1.72 (br d, J=6.50 Hz, 3H))

### Example 156

### 2-((1-(3,6-dimethyl-2-(2-methyl-2H-indazol-5-yl)-4-oxo-41H-chromen-8-yl)ethyl)amino)-N-hydroxybenzamide

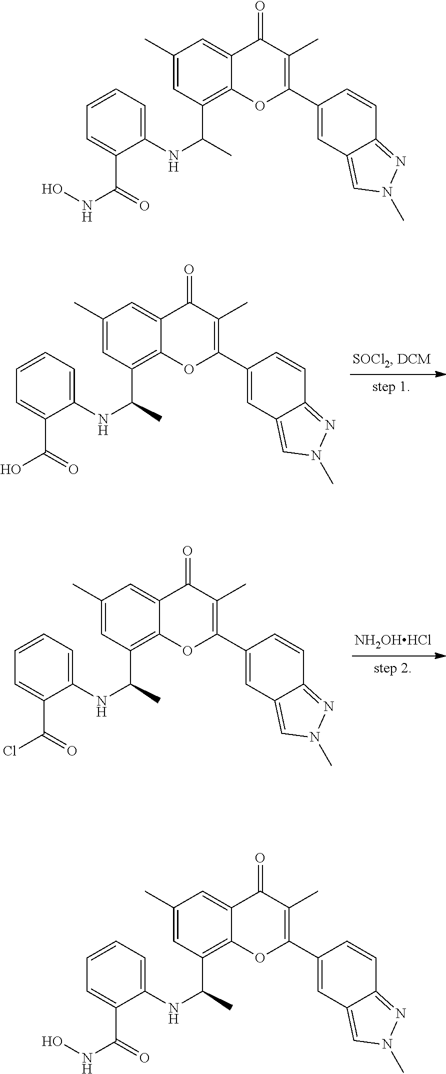 [SMILES: Cc1cc(C(C)Nc2ccccc2C(=O)NO)c2oc(-c3ccc4nn(C)cc4c3)c(C)c(=O)c2c1 ; Cc1cc([C@@H](C)Nc2ccccc2C(=O)O)c2oc(-c3ccc4nn(C)cc4c3)c(C)c(=O)c2c1 ; Cc1cc([C@@H](C)Nc2ccccc2C(=O)Cl)c2oc(-c3ccc4nn(C)cc4c3)c(C)c(=O)c2c1 ; Cc1cc([C@@H](C)Nc2ccccc2C(=O)NO)c2oc(-c3ccc4nn(C)cc4c3)c(C)c(=O)c2c1]

### Step 1

To a solution of 2-[[(1R)-1-[3,6-dimethyl-2-(2-methylindazol-5-yl)-4-oxo-chromen-8-yl]ethyl]amino]benzoic acid (40 mg, 85.56 μmol, 1 eq) in DCM (2 mL) was added thionyl chloride (12.21 mg, 102.67 μmol, 7.45 uL, 1.2 eq). The mixture was stirred at 25° C. for 3 hr. LC-MS showed ˜70% of desired compound was detected. The reaction mixture was concentrated under reduced pressure to give a crude. The crude was used to next step and no more purification. Compound 2-[[(1R)-1-[3,6-dimethyl-2-(2-methylindazol-5-yl)-4-oxo-chromen-8-yl]ethyl]amino]benzoyl chloride (50 mg, crude) was obtained as a red solid.

MS m/z (ESI): 482 [M+H]⁺.

### Step 2

To a solution of 2-[[(1R)-1-[3,6-dimethyl-2-(2-methylindazol-5-yl)-4-oxo-chromen-8-yl]ethyl]amino]benzoyl chloride (40 mg, 82.31 μmol, 1 eq) in THF (2 mL) and H₂O (1 mL) was added NH₂OH·HCl (17.16 mg, 246.93 μmol, 3 eq) and NaHCO₃ (24.20 mg, 288.09 μmol, 11.20 uL, 3.5 eq) at 25° C. for 1 hr. LC-MS showed ˜50% of desired compound was detected. The reaction mixture was concentrated under reduced pressure to give a residue. The residue was purified by prep-HPLC (column: Welch Xtimate C18 150*30 mm*5 um; mobile phase: [water (FA)-ACN]; B %: 18%-58%, 25 min). Compound 2-[[(1R)-1-[3,6-dimethyl-2-(2-methylindazol-5-yl)-4-oxo-chromen-8-yl]ethyl]amino]benzenecarbohydroxamic acid (14.9 mg, 30.03 μmol, 36.48% yield, 97.25% purity) was obtained as a white solid.

¹H NMR (400 MHz, DMSO-d₆) δ ppm 11.13 (br s, 1H) 9.03 (br s, 1H) 8.55 (s, 1H) 8.23 (s, 1H) 8.01 (br d, J=6.38 Hz, 1H) 7.71-7.80 (m, 2H) 7.63 (dd, J=9.01, 1.63 Hz, 1H) 7.54 (d, J=2.00 Hz, 1H) 7.41 (dd, J=7.75, 1.00 Hz, 1H) 7.04-7.16 (m, 1H) 6.53 (s, 1H) 6.44 (d, J=8.25 Hz, 1H) 5.10 (t, J=6.57 Hz, 1H) 4.23 (s, 3H) 2.37 (s, 3H) 2.14 (s, 3H) 1.55 (d, J=6.63 Hz, 3H) MS m/z (ESI): 483 [M+H]⁺.

### Example 157

### (R)—N-(cyclopropylsulfonyl)-2-((1-(6-methyl-2-(2-methyl-2H-indazol-5-yl)-4-oxo-4H-chromen-8-yl)ethyl)amino)benzamide

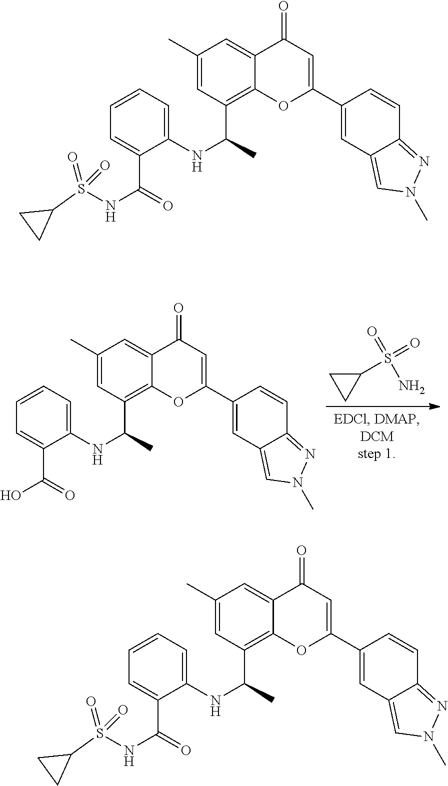

### Step 1

To a solution of cyclopropanesulfonamide (8.02 mg. 66.15 μmol, 1.5 eq) and 2-[[(1R)—1-[6-methyl-2-(2-methylindazol-5-yl)-4-oxo-chromen-8-yl]ethyl]amino]benzoic acid (20.00 ng, 44.10 umol, 1 eq) in DCM (2 mL) was added EDCI (12.68 mg, 66.15 Umol, 1.5 eq) and DMAP (11.85 ng, 97.03 μmol, 2.2 eq). The mixture was stirred at 25° C. for 18 hr. LC-MS showed desired compound was detected. The reaction mixture was concentrated under reduced pressure to remove DCM. The residue was purified by prep-HPLC (column: Welch Xtimate C18 150*30 mm*5 um; mobile phase: [water (FA)-ACN]; B %: 28%-68%, 25 min) to give desired compound (10.1 mg, yield 41.14%, purity 98%) as a white solid.

¹H NMR (400 MHz, DMSO-d₆) δ ppm 11.38-12.26 (m, 1H) 8.55-8.65 (m, 2H) 8.34-8.55 (m, 1H) 7.86-7.97 (m, 1H) 7.70-7.82 (m, 3H) 7.60 (d, J=2.13 Hz, 1H) 7.22-7.34 (m, 1H) 6.95-7.09 (m, 1H) 6.48-6.72 (m, 2H) 5.33-5.46 (m, 1H) 4.22 (s, 3H) 3.02-3.14 (m, 1H) 2.33-2.44 (m, 3H) 1.63-1.80 (m, 3H) 1.08-1.14 (m, 2H) 0.96-1.05 (m, 2H).

MS m/z (ESI): 557 [M+H]⁺.

### Example 158

### (R)-2-((1-(6-methyl-2-(2-methyl-2H-indazol-5-yl)-4-oxo-4H-chromen-8-yl)ethyl)amino)-N-(methylsulfonyl)benzamide

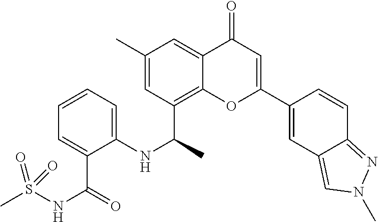

(R)-2-((1-(6-methyl-2-(2-methyl-2H-indazol-5-yl)-4-oxo-4H-chromen-8-yl)ethyl)amino)-N-(methylsulfonyl)benzamide is prepared in accordance with the method of Example 157.

¹H NMR (400 MHz, DMSO-d₆) δ ppm 11.30-12.39 (m, 1H) 8.59 (d, J=18.89 Hz, 2H) 8.23-8.50 (m, 1H) 7.91 (dd, J=9.19, 1.69 Hz, 1H) 7.65-7.84 (m, 3H) 7.51-7.64 (m, 1H) 7.22-7.32 (m, 1H) 6.99-7.09 (m, 1H) 6.46-6.70 (m, 2H) 535-5.45 (m, 1H) 4.23 (s, 3H) 3.32 (s, 3H) 2.38 (s, 3H) 1.72 (d, J=6.63 Hz, 3H) MS m/z (ESI): 531 [M+H]⁺.

### Example 159

### 2-((1-(2-(4-(4-acetylpiperazin-1-yl)phenyl)-3,6-dimethyl-4-oxo-4H-chromen-8-yl)ethyl)amino)benzenesulfonamide

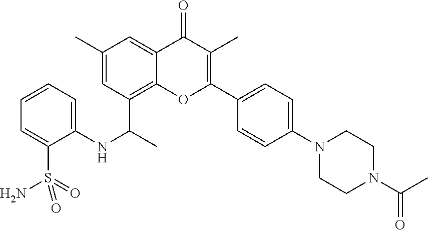 [SMILES: CC(=O)N1CCN(c2ccc(-c3oc4c(C(C)Nc5ccccc5S(N)(=O)=O)cc(C)cc4c(=O)c3C)cc2)CC1]

2-((1-(2-(4-(4-acetylpiperazin-1-yl)phenyl)-3,6-dimethyl-4-oxo-4H-chromen-8-yl)ethyl)amino)benzenesulfonamide is prepared in accordance with the method of Example 131.

¹H NMR (400 MHz, DMSO-d₆) δ ppm 7.75 (s, 1H) 7.71 (d, J=8.88 Hz, 2H) 7.62-7.67 (m, 2H) 7.53-7.60 (m, 2H) 7.16-7.26 (m, 4H) 7.12 (d, J=9.01 Hz, 2H) 6.84-6.91 (m, 1H) 6.62-6.68 (m, 1H) 6.44 (d, J=8.38 Hz, 1H) 6.37 (d, J=5.75 Hz, 1H) 5.33 (t, J=4.75 Hz, 1H) 3.58-3.62 (m, 4H) 2.34 (s, 3H) 2.14 (s, 3H) 2.06 (s, 3H) 1.98-2.03 (n, 4H) 1.60 (br d, J=6.75 Hz, 3H).

MS m/z (ESI): 575 [M+H]⁺.

### Example 160

### 2-(((1R)-1-(3,6-dimethyl-2-(4-((1-methylpyrrolidin-3-yl)oxy)phenyl)-4-oxo-4H-chromen-8-yl)ethyl)amino)benzoic acid

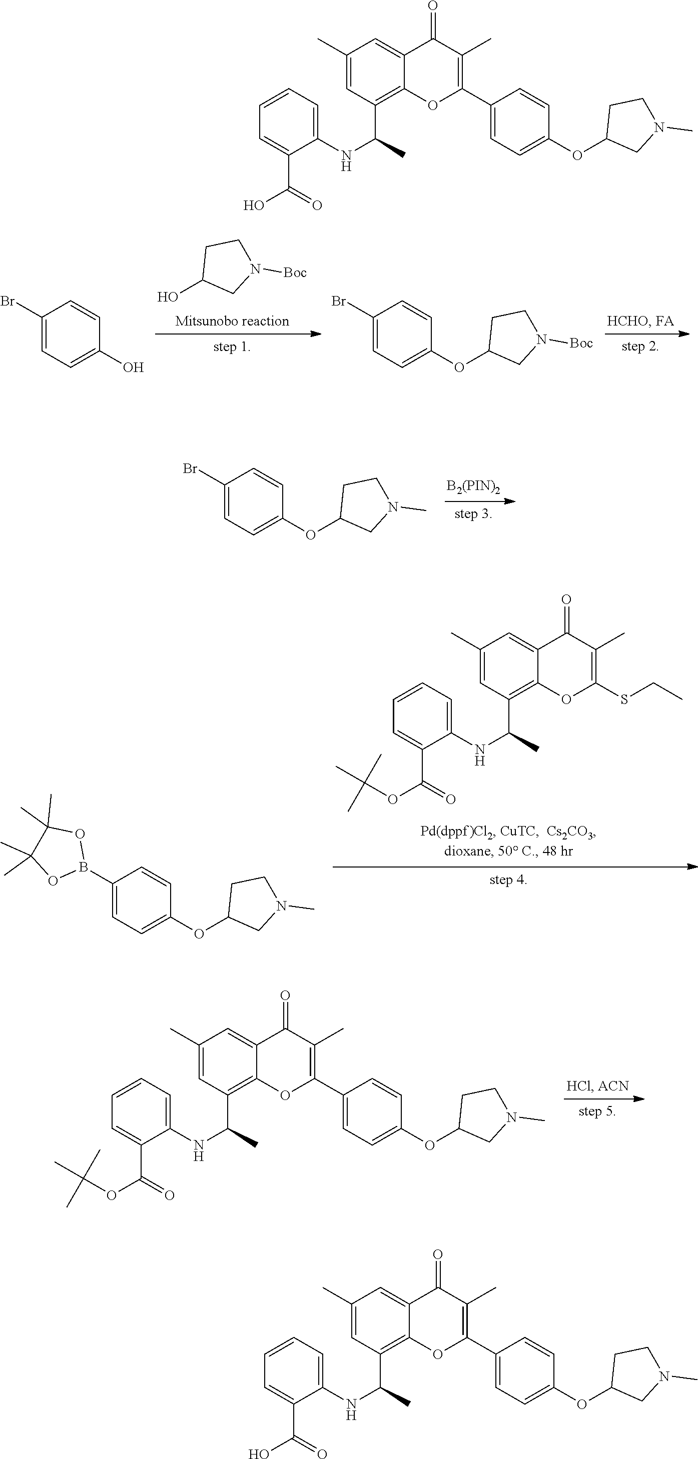

### Step 1

To a solution of 4-bromophenol (2 g, 11.56 mmol, 1 eq) and tert-butyl 3-hydroxypyrrolidine-1-carboxylate (2.60 g, 13.87 mmol, 1.2 eq) in THF (20 mL) was added PPh₃ (3.64 g, 13.87 mmol, 1.2 eq) and DIAD (2.81 g, 13.87 mmol, 2.70 mL, 1.2 eq) under N₂ atmosphere. The mixture was stirred at 25° C. under N₂ atmosphere for 16 hr. LC MS showed one main peak desired mass was detected. The reaction mixture was filtered to remove the insoluble and concentrated under vacuum to give the crude product. The crude product was purified by flash silica gel chromatography (ISCO®; 40 g SepaFlash® Silica Flash Column, Eluent of 0˜20% Ethyl acetate/Petroleum ether gradient @ 80 mL/min). Compound tert-butyl 3-(4-bromophenoxy) pyrrolidine-1-carboxylate (2.93 g, 8.56 mmol, 74% yield) was obtained as a yellow oil.

### Step 2

To a solution of tert-butyl 3-(4-bromophenoxy) pyrrolidine-1-carboxylate (2.7 g, 7.89 mmol, 1 eq) in FORMIC ACID (7 mL) was added HCHO (1.07 g, 35.50 mmol, 977.99 uL, 4.5 eq). The mixture was stirred at 100° C. for 16 hr. LCMS showed one main peak desired mass was detected. The reaction mixture was diluted with H₂O (50 mL) and extracted with ethyl acetate (50 mL*3). The aqueous phase was lyophilized to obtain the crude product. The crude product was directly used to the next step and no further purification. Compound 3-(4-bromophenoxy)-1-methyl-pyrrolidine (2.01 g, crude) was obtained as a brown oil.

¹H NMR (400 MHz, DMSO-d₆) δ ppm 7.43 (br d, J=8.75 Hz, 2H) 6.86 (br d, J=8.63 Hz, 2H) 4.87 (br s, 1H) 4.63-4.73 (m, 1H) 2.64-2.91 (m, 2H) 2.46-2.59 (m, 2H) 2.35 (br s, 3H) 1.77 (br s, 1H)

MS m/z (ESI): 256 [M+H]⁺.

### Step 3

A mixture of 3-(4-bromophenoxy)-1-methyl-pyrrolidine (1 g, 3.90 mmol, 1 eq), 4,4,5,5-tetramethyl-2-(4,4,5,5-tetramethyl-1,3,2-dioxaborolan-2-yl)-1,3,2-dioxaborolane (1.98 g, 7.81 mmol, 2 eq), KOAc (1.15 g, 11.71 mmol, 3 eq), Pd(dppf)Cl₂·CH₂Cl₂ (319 mg, 390.41 μmol, 0.1 eq) in dioxane (5 mL) was degassed and purged with N₂ for 3 times, and then the mixture was stirred at 80° C. for 16 hr under N₂ atmosphere. LCMS showed one main peak desired mass was detected. The reaction mixture was diluted with H₂O (50 mL) and extracted with ethyl acetate (50 mL*3). The combined organic layers were dried over anhydrous Na₂SO₄, filtered and concentrated under reduced pressure to give a crude product. The crude product was purified by prep-HPLC (column: Xtimate C18 150*40 mm*10 um; mobile phase: [water (FA)-ACN]; B %: 0%-32%, 36 min). Compound 1-methyl-3-[4-(4,4,5,5-tetramethyl-1,3,2-dioxaborolan-2-yl)phenoxy]pyrrolidine (75 mg, 247.03 μmol, 6% yield) was obtained as a gray oil.

MS m/z (ESI): 304 [M+H]⁺.

### Step 4

A mixture of 1-methyl-3-[4-(4,4,5,5-tetramethyl-1,3,2-dioxaborolan-2-yl)phenoxy] pyrrolidine (64 mg, 211.64 μmol, 2 eq), tert-butyl 2-[[(1R)-1-(2-ethylsulfanyl-3,6-dimethyl-4-oxo-chromen-8-yl)ethyl]amino]benzoate (48 mg, 105.82 μmol, 1 eq), Cs₂CO₃ (69 mg, 211.64 μmol, 2 eq), Pd(dppf)Cl₂·CH₂Cl₂ (17 mg, 21.16 μmol, 0.2 eq) and thiophene-2-carbonyloxycopper (40.36 mg, 211.64 μmol, 2 eq) in dioxane (3 mL) was degassed and purged with N₂ for 3 times, and then the mixture was stirred at 50° C. for 16 hr under N₂ atmosphere. LCMS showed one main peak desired mass was detected. The reaction mixture was filtered to remove the insoluble and concentrated under vacuum to give the crude product. The crude product was directly used to the next step and no more purification. Compound tert-butyl 2-[[(1R)-1-[3,6-dimethyl-2-[4-(1-methylpyrrolidin-3-yl)oxyphenyl]-4-oxo-chromen-8-yl]ethyl]amino]benzoate (80 mg, crude) was obtained as a black oil.

MS mm/z (ESI): 569 [M+H]⁺.

### Step 5

To a solution of tert-butyl 2-[[(1R)-1-[3,6-dimethyl-2-[4-(1-methylpyrrolidin-3-yl)oxyphenyl]-4-oxo-chromen-8-yl]ethyl]amino]benzoate (70 mg, 123.09 μmol, 1 eq) in ACN (3 mL) was added HCl (12 M, 61.54 uL, 6 eq). The mixture was stirred at 80° C. for 2 hr. LC MS showed one main peak desired mass was detected. The reaction mixture was filtered to remove the insoluble and concentrated under vacuum to give the crude product The crude product was purified by prep-HPLC (column: Welch Xtimate C18 150*30 mm*5 um; mobile phase: [water (FA)-ACN]; 1B %: 2%-42%, 25 min). Compound 2-[[(1R)-1-[3, 6-dimethyl-2-[4-(1-methylpyrrolidin-3-yl) oxyphenyl]-4-oxo-chromen-8-yl]ethyl]amino]benzoic acid (II mg, 18.04 μmol, 15% yield, 96% purity, FA) was obtained as a white solid.

¹H NMR (400 MHz, DMSO-d₆) ppm 8.43 (br d, J=4.38 Hz, 1H) 8.15 (s, 1H) 7.81 (dd, J=7.94, 1.44 Hz, 1H) 7.74 (d, J=9.01 Hz, 3H) 7.51 (d, J=1.88 Hz, 1H) 7.15-7.24 (n, 1H) 7.07 (d, J=8.88 Hz, 2H) 6.54 (t, J=7.44 Hz, 1H) 6.44 (d, J=8.38 Hz, 1H) 5.13 (br d, J=5.38 Hz, 1H) 4.98 (br t, J=6.50 Hz, 1H) 2.84 (br dd, J=10.32, 5.82 Hz, 1H) 2.66-2.76 (m, 3H) 2.41 (br d, J=7.38 Hz, 1H) 2.36 (s, 3H) 2.30 (s, 3H) 2.10 (s, 3H) 1.78-1.87 (m, 1H) 1.58 (d, J=6.63 Hz, 3H)

MS m/z (ESI): 513 [M+H]⁺.

### Example 161

### ((R)-2-((1-(2-(4-(4-(dimethylamino)piperidin-1-yl)phenyl)-3,6-dimethyl-4-oxo-4H-chromen-8-yl)ethyl)amino)benzoic acid

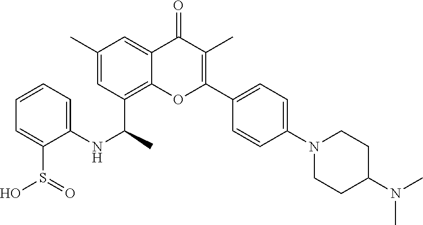 [SMILES: Cc1cc([C@@H](C)Nc2ccccc2S(=O)O)c2oc(-c3ccc(N4CCC(N(C)C)CC4)cc3)c(C)c(=O)c2c1]

(R)-2-((1-(2-(4-(4-(dimethylamino)piperidin-1-yl)phenyl)-3,6-dimethyl-4-oxo-41-chromen-8-yl)ethyl)amino)benzoic acid is prepared in accordance with the method of Example 133.

¹H NMR (400 MHz, ACETONITRILE-d₃) δ ppm 8.57-8.69 (m, 1H) 7.80 (dd, J=7.82, 1.56 Hz, 1H) 7.74 (s, 1H) 7.57-7.65 (m, 2H) 7.49 s, 1H) 7.11-7.19 (m, 1H) 7.07 (br d, J=8.88 Hz, 2H) 6.50 (t, J=7.50 Hz, 1H) 6.40 (d, J=8.75 Hz, 1H) 5.09 (br d, J=0.88 Hz, 1H) 3.91-4.00 (m, 2H) 285 (br t, J=11.57 Hz, 2H) 2.51-2.58 (m, 3H) 2.28-2.40 (m, 9H) 2.07-2.14 (m, 3H) 1.86-1.94 (m, 2H) 1.54 (br d, J=6.00 Hz, 3H) MS m/z (ESI): 540 [M+H]⁺.

### Example 162

### (R)-2-((1-(3,6-dimethyl-2-(4-(1-methyl-1H-imidazol-4-yl)phenyl)-4-oxo-41H-chromen-8-yl)ethyl)amino)benzoic acid

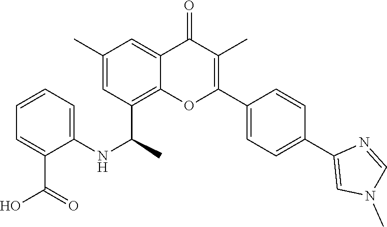 [SMILES: Cc1cc([C@@H](C)Nc2ccccc2C(=O)O)c2oc(-c3ccc(-c4cn(C)cn4)cc3)c(C)c(=O)c2c1]

(R)-2-((1-(3,6-dimethyl-2-(4-(1-methyl-1H-imidazol-4-yl)phenyl)-4-oxo-4H-chromen-8-yl)ethyl)amino)benzoic acid is prepared in accordance with the method of Example 149.

¹H NMR (400 MHz, DMSO-d₆) δ ppm 8.24-8.49 (m, 1H) 8.02 (s, 1H) 7.74-7.88 (m, 4H) 7.55 (d, J=2.00 Hz, 1H) 7.42 (t, J=7.69 Hz, 2H) 7.28 (s, 1H) 7.15-7.22 (m, 1H) 6.54 (t, J=7.44 Hz, 1H) 6.40 (d, J=8.38 Hz, 1H) 5.18 (br d, J=6.75 Hz, 11H) 3.97 (s, 3H) 2.37 (s, 3H) 2.22 (s, 3H) 1.58 (d, J=6.50 Hz, 3H).

MS m/z (ESI): 494 [M+H]⁺.

### Example 163

### R)-2-((1-(3,6-dimethyl-2-(6-(1-methyl-1H-imidazol-4-yl)pyridin-3-yl)-4-oxo-411-chromen-8-yl)ethyl)amino)benzoic acid

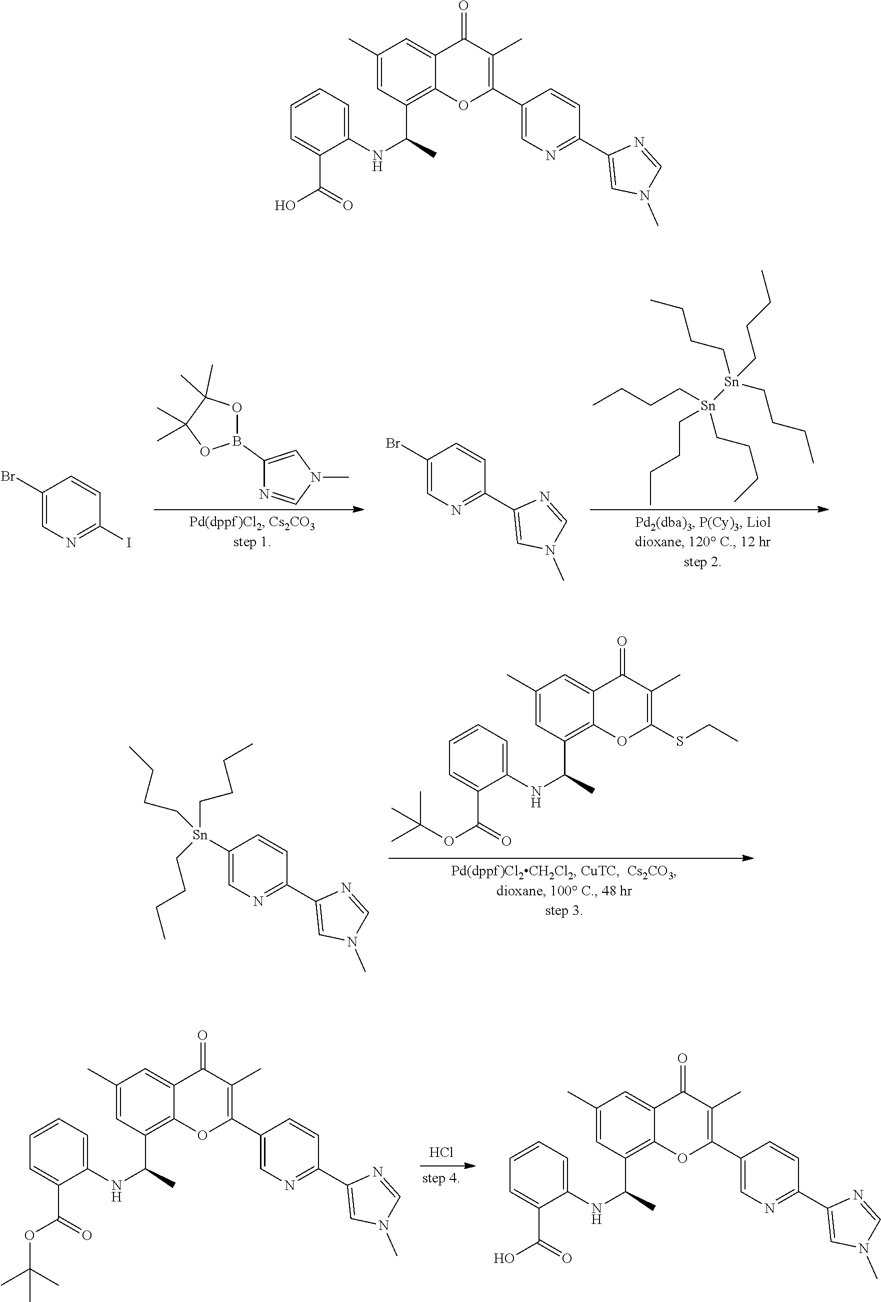

### Step 1

A mixture of 5-bromo-2-iodo-pyridine (2 g, 7.04 mmol, 1 eq), tributyl-(1-methylimidazol-4-yl)stannane (2 g, 7.04 mmol, 1 eq), Cs₂CO₃ (4 g, 14.09 mmol, 2 eq), Pd(dppf)Cl₂·CH₂Cl₂ (1 g, 1.41 mmol, 0.2 eq) in toluene (30 mL) and H₂O (10 mL) was degassed and purged with N₂ for 3 times, and then the mixture was stirred at 100° C. for 12 hr under N₂ atmosphere. LCMS showed desired mass was detected. The reaction mixture was extracted with dichloromethane (50 ml*3). The combined organic layers were dried over anhydrous Na₂SO₄, filtered and concentrated under reduced pressure to give a crude product. The crude product was purified by flash silica gel chromatography (ISCO®; 40 g SepaFlash® Silica Flash Column, Eluent of 0-70% Ethyl acetate/Petroleum ether gradient @ 50 mL/min). Compound 5-bromo-2-(1-methylimidazol-4-yl) pyridine (1 g, 3.57 mmol, 51% yield) was obtained as White solid.

MS m/z (ESI): 238 [M+H]⁺.

### Step 2

A mixture of 5-bromo-2-(1-methylimidazol-4-yl)pyri dine (200 mg, 840.04 umol, 758.62 uL, 1 eq), tributyl(tributylstannyl)stannane (1 g, 2.10 mmol, 1.05 mL, 2.5 eq), Pd₂(dba)₃ (76 mg, 84.00 umol, 0.1 eq), P(Cy)₃ (47 mg, 168.01 umol, 54.47 uL, 0.2 eq) and LiCl (178 mg, 4.20 mmol, 86.01 uL, 5 eq) in dioxane (5 mL) was degassed and purged with N₂ for 3 times, and then the mixture was stirred at 120° C. for 12 hr under N₂ atmosphere. LCMS showed desired mass was detected. The reaction mixture was quenched with KF (aq.) (10 ml) and extracted with ethyl acetate (20 ml*3). The combined organic layers were dried over anhydrous Na₂SO₄, filtered and concentrated under reduced pressure to give a crude product. The crude product was purified by flash silica gel chromatography (ISCO®; 4 g SepaFlash® Silica Flash Column, Eluent of 0-50% Ethyl acetate/Petroleum ether gradient @ 25 mL/min). Compound tributyl-[6-(1-methylimidazol-4-yl)-3-pyridyl]stannane (200 mg, 419.43 umol, 50% yield) was obtained as a white solid.

MS mm/z (ESI): 450 [M+H]⁺.

### Step 3

A mixture of tert-butyl 2-[[(1R)-1-(2-ethylsulfanyl-3,6-dimethyl-4-oxo-chromen-8-yl)ethyl]amino]benzoate (100 mg, 220.46 umol, 1 eq), tributyl-[6-(1-methylimidazol-4-yl)-3-pyridyl]stannane (197 mg, 440.92 umol, 2 eq), Cs₂CO₃ (143.66 mg, 440.92 umol, 2 eq), Pd(dppf)Cl₂·CH₂Cl₂ (36.01 mg, 44.09 umol, 0.2 eq) and thiophene-2-carbonyloxycopper (84 mg, 440.92 umol, 2 eq) in dioxane (5 mL) was degassed and purged with N₂ for 3 times, and then the mixture was stirred at 50° C. for 12 hr under N₂ atmosphere. LCMS showed desired mass was detected. The reaction mixture was extracted with ethyl acetate (20 ml*3). The combined organic layers were dried over anhydrous Na₂SO₄, filtered and concentrated under reduced pressure to give a crude product. The crude product was purified by flash silica gel chromatography (ISCO®; 4 g SepaFlash® Silica Flash Column, Eluent of 0˜70% Ethyl acetate/Petroleum ether gradient @ 30 mL/min). Compound tert-butyl 2-[[(1R)-1-[3, 6-dimethyl-2-[6-(1-methylimidazol-4-yl)-3-pyridyl]-4-oxo-chromen-8-yl] ethyl] amino] benzoate (70 mg, 127.12 umol, 58% yield) was obtained as a white solid.

MS m/z (ESI): 551 [M+H]⁺.

### Step 4

To a solution of tert-butyl 2-[[(1R)-1-[3, 6-dimethyl-2-[6-(1-methylimidazol-4-yl)-3-pyridyl]-4-oxo-chromen-8-yl] ethyl] amino] benzoate (50 mg, 90.80 umol, 1 eq) in ACN (1 mL) was added HCl(aq.) (12 M, 30.27 uL, 4 eq). The mixture was stirred at 80° C. for 1 hr. LCMS showed desired mass was detected. The reaction mixture was concentrated under vacuum to give the crude product. The reaction mixture without purification. Compound 2-[[(1R)-1-[3, 6-dimethyl-2-[6-(1-methylimidazol-4-yl)-3-pyridyl]-4-oxo-chromen-8-yl] ethyl] amino] benzoic acid (30 mg, 60.66 umol, 66% yield) was obtained as a white solid.

¹H NMR (400 MHz, DMSO-d₆) δ ppm 8.99 (br s, 1H) 8.17-8.38 (m, 2H) 8.03 (br d, J=8.25 Hz, 1H) 7.82-7.94 (m, 2H) 7.77 (br s, 2H) 7.56 (br s, 1H) 7.09 (br t, J=7.25 Hz, 1H) 6.48 (br t, J==7.32 Hz, 1H) 6.41 (br d, J=7.75 Hz, 1H) 5.11 (br d, J=2.75 Hz, 1H) 3.76 (s, 3H) 2.36 (s, 3H) 2.15 (s, 3H) 1.58 (br d, J=6.25 Hz, 3H) MS m/z (ESI): 495 [M+H]⁺.

### Example 164

### (R)-2-((1-(6-methyl-2-(6-(1-methyl-1H-pyrazol-4-yl)pyridin-3-yl)-4-oxo-4H-chromen-8-yl)ethyl)amino)benzoic acid

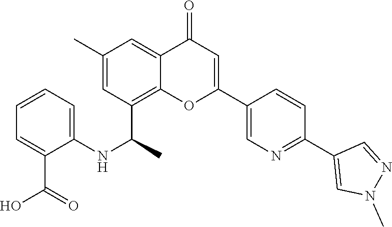 [SMILES: Cc1cc([C@@H](C)Nc2ccccc2C(=O)O)c2oc(-c3ccc(-c4cnn(C)c4)nc3)cc(=O)c2c1]

(R)-2-((1-(6-methyl-2-(6-(1-methyl-1H-pyrazol-4-yl)pyridin-3-yl)-4-oxo-4H-chromen-8-yl)ethyl)amino)benzoic acid is prepared in accordance with the method of Example 150.

¹H NMR (400 MHz, DMSO-d's) ppm 9.24 (d, J=1.75 Hz, 1H) 8.35-8.56 (m, 2H) 8.13 (s, 1H) 7.79-7.97 (m, 2H) 7.74 (s, 1H) 7.54 (s, 1H) 7.19 (s, 1H) 6.93-7.10 (m, 1H) 6.42-6.53 (m, 1H) 6.28-6.41 (m, 1H) 5.26-5.38 (m, 1H) 3.92 (s, 3H) 2.35 (s, 3H) 1.64 (br d, J=6.50 Hz, 3H).

MS m/z (ESI): 481 [M+H]⁺.

### Example 165

### (R)-6-chloro-3-((1-(2-(4-(2-(dimethylamino)ethoxy)phenyl)-3,6-dimethyl-4-oxo-41-1 chromen-8-yl)ethyl)amino)picolinic acid

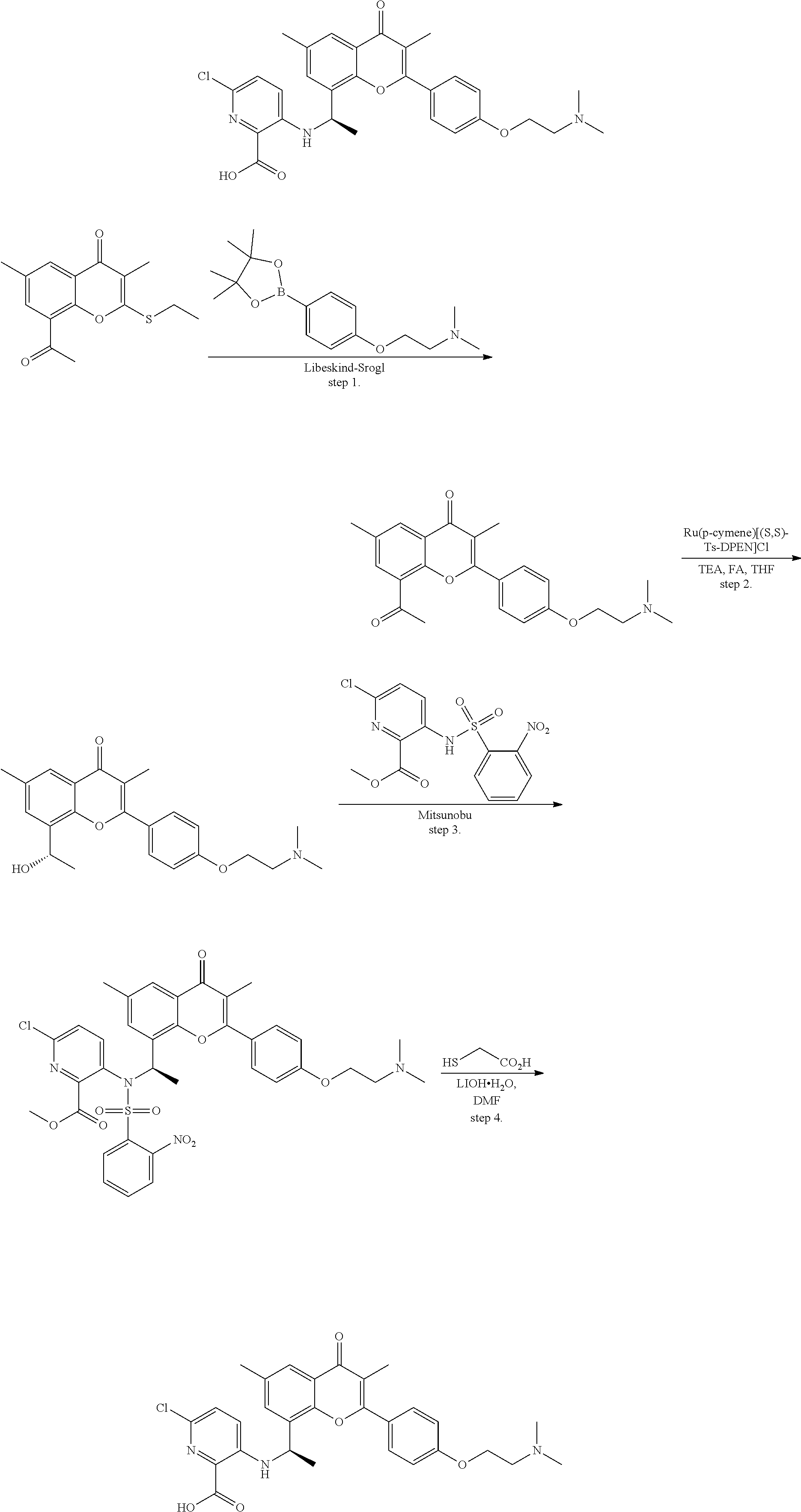

### Step 1

To a solution of 8-acetyl-2-ethylsulfanyl-3,6-dimethyl-chromen-4-one (250 mg, 904.65 umol, 1 eq), N,N-dimethyl-2-[4-(4,4,5,5-tetramethyl-1,3,2-dioxaborolan-2-yl)phenoxy] ethanamine (316 mg, 1.09 mmol, 1.2 eq), Cs₂CO₃ (590 mg, 1.81 mmol, 2 eq), thiophene-2-carbonyloxycopper (345 mg, 1.81 mmol, 2 eq) and Pd(dppf)Cl₂·CH₂Cl₂ (148 mg, 180.93 umol, 0.2 eq) in dioxane (6 mL) was degassed and purged with N₂ for 3 times, and then the mixture was stirred at 65° C. for 48 hr under N₂ atmosphere. The reaction mixture was diluted with H₂O (50 mL) and extracted with ethyl acetate (50 mL*3). The combined organic layers were dried over [Na₂SO₄], filtered and concentrated under reduced pressure to give a crude product. The crude product was purified by flash silica gel chromatography (ISCO®; 4 g SepaFlash® Silica Flash Column, Fluent of 0˜100% Ethyl acetate/Petroleum ether gradient (18 mL/min, ISCO®; 1 g SepaFlash® Silica Flash Column, Eluent of 0-20% Methanol/dichloromethane gradient @ 18 mL/min). Compound 8-acetyl-2-[4-[2-(dimethylamino)ethoxy]phenyl]-3,6-dimethyl-chromen-4-one (200 mg, 527.08 umol, 58% yield) was obtained as a yellow solid.

MS m/z (ES1): 380[M+H]⁺.

### Step 2

To a solution of TEA (120 mg, 1.19 mmol, 165 μL, 3 eq) in THF (0.5 mL) was added dropwise formic acid (91 mg, 1.98 mmol, 75 μL, 5 eq) at 0° C., the mixture was stirred at 25° C. for 10 min to give the triethylammonium formate solution. To a solution of 8-acetyl-2-[4-[2-(dimethylamino)ethoxy]phenyl]-3,6-dimethyl-chromen-4-one (150 mg, 395.31 umol, 1 eq) and N-[(1 S,2S)-2-amino-1,2-diphenyl-ethyl]-4-methyl-benzene sulfonamide; chlororuthenium; 1-isopropyl-4-methyl-benzene (25 mg, 39.53 umol, 0.1 eq) in THF (2 mL) was added dropwise the triethylammonium formate solution at 25° C., the mixture was degassed and purged with N₂ for 3 times and stirred at 60° C. for 16 hr. The reaction mixture was concentrated under vacuum to give the crude product. The crude product was purified by flash silica gel chromatography (ISCO®; 4 g SepaFlash® Silica Flash Column, Eluent of 0˜100% Ethyl acetate/Petroleum ether gradient @18 mL/min, Eluent of 0˜-15% Methanol/dichloromethane gradient (@ 18 mL/min). Compound 2-[4-[2-(dimethylamino)ethoxy]phenyl]-8-[(1 S)-1-hydroxyethyl]-3,6-dimethyl-chromen-4-one (145 mg, 380.11 umol, 96% yield) was obtained as a black oil.

MS m/z (ESI): 382[M+H]⁺.

### Step 3

To a solution of 2-[4-[2-(dimethylamino)ethoxy]phenyl]-8-[(1S)-1-hydroxyethyl]-3,6-dimethyl-chromen-4-one (145 mg, 380.11 umol, 1 eq), methyl 6-chloro-3-[(2-nitrophenyl)sulfonylamino]pyridine-2-carboxylate (170 mg, 456.14 umol, 1.2 eq) and PPh₃ (299 mg, 1.14 mmol, 3 eq) in THF (2 mL). The mixture was stirred at 25° C. for 0.5 hr. Then DIAD (231 mg, 1.14 mmol, 222 μL, 3 eq) in THF (0.25 mL) was added to the mixture at 0° C., the mixture was degassed and purged with N₂ for 3 times, and then the mixture was stirred at 25° C. for 16 br under N₂ atmosphere. The reaction mixture was filtered to remove the insoluble to give the crude product. The crude product was purified by prep-HPLC (FA condition, column: Welch Xtimate C18 150*30 mm*Sum; mobile phase: [water (FA)-ACN]; B %: 4%-44%, 25 min). Compound methyl 6-chloro-3-[[(1R)-1-[2-[4-[2-(dimethylamino)ethoxy]phenyl]-3,6-dimethyl-4-oxo-chromen-8-yl]ethyl]-(2-nitrophenyl)sulfonyl-amino]pyridine-2-carboxylate (130 mg, 176.82 umol, 47% yield) was obtained as a white solid.

MS m/z (ESI): 735[M+H]⁺.

### Step 4

To a solution of methyl 6-chloro-3-[[(1R)-1-[2-[4-[2-(dimethylamino)ethoxy]phenyl]-3, 6-dimethyl-4-oxo-chromen-8-yl]ethyl]-(2-nitrophenyl)sulfonyl-amino]pyridine-2-carboxylate (50 mg, 68.01 umol, 1 eq) in DMF (1 mL) was added LiOH·H₂O (23 mg, 544.07 umol, 8 eq), then ethanethioic S-acid (21 mg, 272.03 umol, 20 μL, 4 eq) was added to the mixture tinder N₂ atmosphere. The mixture was stirred at 80° C. for 3 hr. The reaction mixture was filtered to remove the insoluble to give the crude product. The crude product was purified by prep-HPLC (FA condition, column: Welch Xtimate C18 150*30 mm*5 um; mobile phase: [water (FA)-ACN]; B %: 0%-40%, 25 min). Compound 6-chloro-3-[[(1R)-1-[2-[4-[2-(dimethylamino) ethoxy]phenyl]-3,6-dimethyl-4-oxo-chromen-8-yl]ethyl]amino]pyridine-2-carboxylic acid (18.6 mg, 30.89 umol, 45% yield, 96% purity, FA) was obtained as a white solid.

¹H NMR (400 MHz, DMSO-d₆) δ ppm 8.82 (br dd, J=3.19, 1.81 Hz, 1H) 8.15 (s, 1H) 7.69-7.78 (m, 3H) 7.50 (d, J=1.88 Hz, 1H) 7.18 (d, J=8.76 Hz, 1H) 7.12 (d, J=8.88 Hz, 2H) 6.92 (d, J=9.01 Hz, 1H) 5.09 (br t, J=5.94 Hz, 1H) 4.25 (t, J=5.44 Hz, 2H) 2.96 (br t, J=5.00 Hz, 2H) 2.45 (s, 6H) 2.35 (s, 3H) 2.08 (s, 3H) 1.57 (d, J=6.63 Hz, 3H) MS m/z (ESI): 536[M+H]⁺.

### Example 166

### (R)-2-((1-(2-(4-(2-(dimethylamino)ethoxy)phenyl)-6-methyl-4-oxo-4H-1-chromen-8-yl)ethyl)amino)benzoic acid

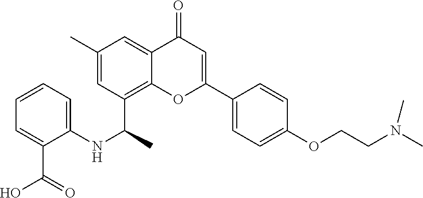 [SMILES: Cc1cc([C@@H](C)Nc2ccccc2C(=O)O)c2oc(-c3ccc(OCCN(C)C)cc3)cc(=O)c2c1]

(R)-2-((l-(2-(4-(2-(dimethylamino)ethoxy)phenyl)-6-methyl-4-oxo-4H-chromen-8-yl)ethyl)amino)benzoic acid is prepared in accordance with the method of Example 164.

¹H NMR (400 MHz, DMSO-d₆) δ ppm 8.54 (br d, J=4.63 Hz, 1H) 8.08 (d, J=9.01 Hz, 2H) 7.82 (dd, J=7.94, 1.56 Hz, 1H) 7.74 (d, J=1.25 Hz, 1H) 7.55 (d, J=2.001 Hz, 1H) 7.18-7.24 (m, 1H) 7.15 (s, 1H) 7.12 (s, 1H) 6.98 (s, 1H) 6.45-6.59 (n 2H) 5.28-5.35 (n, 1H) 4.18 (t, J=5.75 Hz, 2H) 2.70 (t, J=5.69 Hz, 2H) 2.36 (s, 3H) 2.26 (s, 6H) 1.67 (d, J=6.63 Hz, 3H).

MS m/z (ESI): 487 [M+H]⁺.

### Example 167

### 2-(((1R)-1-(3,6-dimethyl-2-(4-(4-methylmorpholin-2-yl)phenyl)-4-oxo-4H-chromen-8-yl)ethyl)amino)benzoic acid

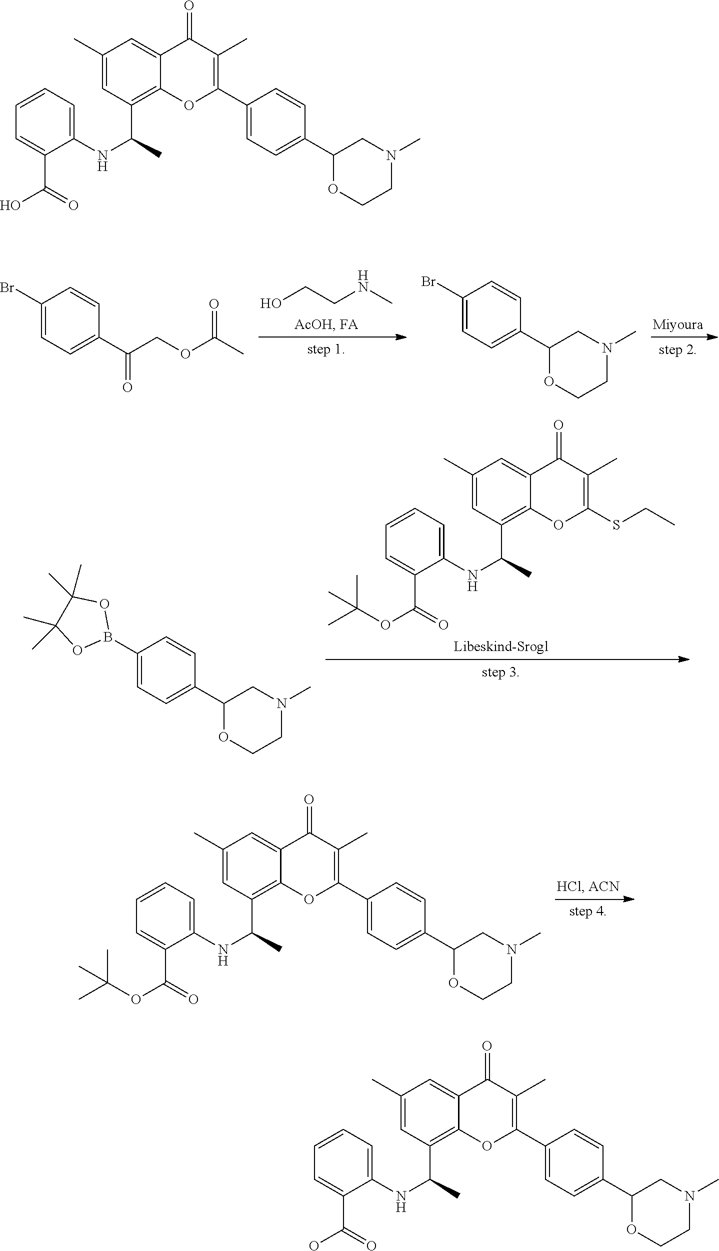

### Step 1

Compound 2-(4-bromophenyl)-4-methyl-morpholine (560 mg, crude) was obtained as a yellow solid used the method in Chem. Ber 1982, 115, 2635-2642.

¹H NMR (400 MHz, DMSO-d₆) δ ppm 7.52 (d, J=8.38 Hz, 2H) 7.31 (d, J=8.38 Hz, 2H) 4.46 (dd, J=10.13, 2.25 Hz, 1H) 3.92 (dt, J=9.69, 1.72 Hz, 1H) 3.65 (td, J=11.38, 2.50 Hz, 1H) 2.83 (br d, J=11.26 Hz, 1H) 2.62-2.71 (m, 1H) 2.19 (s, 3H) 2.04 (td, J=11.48, 3.31 Hz, 1H) 1.79 (t, J=10.76 Hz, 1H)

MS m/z (ESI): 256 [M+H]⁺.

### Step 2

To a solution of 2-(4-bromophenyl)-4-methyl-morpholine (560 mg, 2.19 mmol, 1 eq), 4,4,5,5-tetramethyl-2-(4,4,5,5-tetramethyl-1,3,2-dioxaborolan-2-yl)-1,3,2-dioxaborolane (1.11 g, 4.37 mmol, 2 eq), KOAc (644 mg, 6.56 mmol, 3 eq) and Pd(dppf)Cl₂—CH₂Cl₂ (179 mg, 218.63 μmol, 0.1 eq) in dioxane (6 mL) was degassed and purged with N₂ for 3 times, and then the mixture was stirred at 80° C. for 16 hr under N₂ atmosphere. The crude product was purified by flash silica gel chromatography (ISCO®; 4 g SepaFlash®7 Silica Flash Column, Eluent of 0-40% Ethyl acetate/Petroleum ether gradient @ 30 mL/min). Compound 4-methyl-2-[4-(4,4,5,5-tetramethyl-1,3,2-dioxaborolan-2-yl)phenyl]morpholine (225 mg, 742.08 μmol, 34% yield) was obtained as a yellow oil.

MS m/z (ESI): 304[M+H]⁺.

### Step 3

To a solution of tert-butyl 2-[[(1R)-1-(2-ethylsulfanyl-3,6-dimethyl-4-oxo-chromen-8-yl)ethyl]amino]benzoate (45 mg, 99.21 μmol, 1 eq), 4-methyl-2-[4-(4,4,5,5-tetramethyl-1,3,2-dioxaborolan-2-yl)phenyl] morpholine (120 mg, 396.83 μmol, 4 eq), Cs₂CO₃ (65 mg, 198.42 μmol, 2 eq), thiophene-2-carbonyloxycopper (38 mg, 198.42 μmol, 2 eq) and Pd(dppf)Cl₂ (16 mg, 19.84 μmol, 0.2 eq) in dioxane (2 mL) was degassed and purged with N₂ for 3 times, and then the mixture was stirred at 60° C. for 16 hr under N2 atmosphere. The reaction mixture was filtered to remove the insoluble and concentrated under vacuum to give the crude product. The crude product was purified by flash silica gel chromatography (ISCO®; 4 g SepaFlash® Silica Flash Column, Eluent of 0-100% Ethyl acetate/Petroleum ether gradient @. 18 mL/min). Compound tert-butyl 2-[[(1R)-1-[3,6-dimethyl-2-[4-(4-methylmorpholin-2-yl)phenyl]-4-oxo-chromen-8-yl]ethyl]amino]benzoate (55 mg, 96.71 μmol, 97% yield) was obtained as a yellow oil.

MS mm/z (ESI): 569 [M+H]⁺.

### Step 4

To a solution of tert-butyl 2-[[(1R)-1-[3,6-dimethyl-2-[4-(4-methylmorpholine-2-yl)phenyl]-4-oxo-chromen-8-yl]ethyl]amino]benzoate (50 mg, 87.92 μmol, 1 eq) in MeCN (1 mL) was added HCl(aq.)(12 M, 30 μL, 4 eq). The mixture was stirred at 80° C. for 1 hr. The reaction mixture was filtered to remove the insoluble to give the crude product. The crude product was purified by prep-HPLC (FA condition, column: Welch Xtimate C18 150*30 mm*5 um; mobile phase: [water (FA)-ACN]; gradient: 2%-42% over 25 min). Compound 2-[[(1R)-1-[3,6-dimethyl-2-[4-(4-methylmorpholin-2-yl)phenyl]-4-oxo-chromen-8-yl]ethyl]amino]benzoic acid (9.4 mg, 17.45 μmol, 20% yield, 95% purity) was obtained as a white solid.

¹H NMR (400 MHz, DMSO-d₆) δ ppm 8.35 (br d, J=600 Hz, 1H) 7.75-7.83 (m, 4H) 7.57 (br d, J=7.88 Hz, 2H) 7.52 (s, 1H) 7.20 (br t, J=7.94 Hz, 1H) 6.55 (t, J=7.57 Hz, 1H) 6.44 (d, J=8.63 Hz, 1H) 5.11 (br t, J=6.19 Hz, 1H) 4.51-4.73 (m, 1H) 3.95-4.06 (m, 1H) 3.70-377 (m, 1H) 2.92-3.11 (m, 2H) 2.45 (br s, 3H) 2.36 (s, 3H) 2.29 (br s, 2H) 2.08 (s, 3H) 1.58 (br d, J=6.50 Hz, 3H)

MS m/z (ESI): 513 [M+H]⁺.

### Example 168

### 2-(((R)-1-(2-(4-((S)-3-(dimethylamino)piperidin-1-yl)phenyl)-3,6-dimethyl-4-oxo-41H-chromen-8-yl)ethyl)amino)benzoic acid

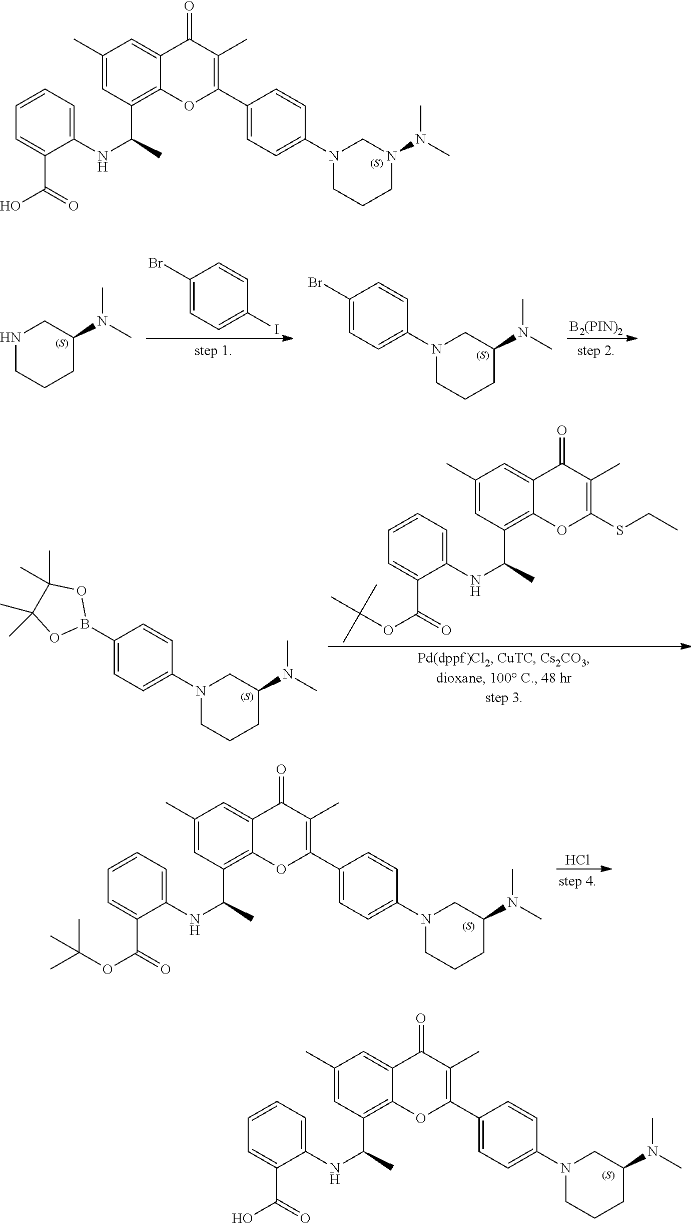

### Step 1

A mixture of 1-bromo-4-iodo-benzene (1.87 g, 6.61 mmol, 1 eq), (3S)—N,N-dimethyl piperidin-3-amine (544 mg, 3.31 mmol, 0.5 eq, HCl), Pd₂(dba)₃ (605 mg, 661.00 umol, 0.1 eq), Cs₂CO₃ (4.31 g, 13.22 mmol, 2 eq) and Xantphos (382 mg, 661.00 umol, 0.1 eq) in dioxane (20 mL) was degassed and purged with N₂ for 3 times, and then the mixture was stirred at 80° C. for 12 hr under N₂ atmosphere. The reaction mixture was filtered to remove the insoluble and concentrated under vacuum to give the crude product. The crude product was purified by flash silica gel chromatography (ISCO®; 20 g SepaFlash® Silica Flash Column, Eluent of 0˜90% Ethyl acetate/Petroleum ether gradient @ 50 mL/min). Compound (3S)-1-(4-bromophenyl)-N, N-dimethyl-piperidin-3-amine (240 mg, 847.44 umol, 13% yield, N/A purity) was obtained as a yellow oil.

MS m/z (ESI): 285 [M+H]⁺.

### Step 2

A mixture of (3S)-1-(4-bromophenyl)-N,N-dimethyl-piperidin-3-amine (280 mg, 988.68 umol, 1 eq), 4,4,5,5-tetramethyl-2-(4,4,5,5-tetramethyl-1,3,2-dioxaborolan-2-yl)-1,3,2-dioxaborolane (502 mg, 1.98 mmol, 2 eq), KOAc (291 mg, 2.97 mmol, 3 eq) and Pd(dppf)Cl₂·CH₂Cl₂ (80 mg, 98.87 umol, 0.1 eq) in dioxane (2 mL) was degassed and purged with N₂ for 3 times, and then the mixture was stirred at 80° C. for 2 h under N₂ atmosphere. The reaction mixture was filtered to remove the insoluble and concentrated under vacuum to give the crude product. The crude product was purified by flash silica gel chromatography (ISCO®; 12 g SepaFlash® Silica Flash Column, Eluent of 0-90% Ethyl acetate/Petroleum ether gradient @ 50 mL/min). Compound (3S)—N, N-dimethyl-1-[4-(4, 4, 5, 5-tetramethyl-1, 3, 2-dioxaborolan-2-yl) phenyl] piperidin-3-amine (240 mg, 726.68 umol, 73.50% yield) was obtained as a yellow oil MS m/z (ESI): 331 [M+H]⁺.

### Step 3

A mixture of tert-butyl 2-[[(1R)-1-(2-ethyl sulfanyl-3,6-dimethyl-4-oxo-chromen-8-yl) ethyl]amino]benzoate (40 mg, 88.18 umol, 1 eq), (3S)—N,N-dimethyl-1-[4-(4,4,5,5-tetramethyl-1,3,2-dioxaborolan-2-yl)phenyl]piperidin-3-amine (58 mg, 176.37 umol, 2 eq), Cs₂CO₃ (57 mg, 176.37 umol, 2 eq), thiophene-2-carbonyloxycopper (33 mg, 176.37 umol, 2 eq) and Pd(dppf)Cl₂·CH₂Cl₂ (14 mg, 17.64 umol, 0.2 eq) in dioxane (5 mL) was degassed and purged with N₂ for 3 times, and then the mixture was stirred at 50° C. for 48 hr under N₂ atmosphere. The reaction mixture was filtered to remove the insoluble and concentrated under vacuum to give the crude product. The crude product was purified by flash silica gel chromatography (ISCO®; 4 g SepaFlash® Silica Flash Column, Eluent of 0-92% Ethyl acetate/Petroleum ether gradient @ 50 mL/min). Compound tert-butyl 2-[[(1R)-1-[2-[4-[(3S)-3-(dimethylamino)-1-piperidyl] phenyl]-3, 6-dimethyl-4-oxo-chromen-8-yl] ethyl] amino] benzoate (18 mg, 30.21 umol, 34% yield, N/A purity) was obtained as a white solid.

MS m/z (ESI): 596 [M+H]⁺.

### Step 4

To a solution of tert-butyl 2-[[(1R)-1-[2-[4-[(3S)-3-(dimethylamino)-1-piperidyl]phenyl]-3,6-dimethyl-4-oxo-chromen-8-yl]ethyl]amino]benzoate (18 mg, 30.21 umol, 1 eq) in ACN (2 mL) was added HCl (12 M, 10 μL, 4 eq). The mixture was stirred at 80° C. for 2 hr. The reaction mixture was filtered to remove the insoluble and concentrated under vacuum to give the crude product. The crude product was purified by prep-HPLC (column: Welch Xtimate C18 150*30 mm.*5 um; mobile phase: [water (FA)-ACN]; B %: 4%-44%, 25 min). Compound 2-[[(1R)-1-[2-[4-[(3S)-3-(dimethylamino)-1-piperidyl] phenyl]-3, 6-dimethyl-4-oxo-chromen-8-yl] ethyl] amino] benzoic acid (12.4 mg, 21.97 umol, 73% yield, 96% purity) was obtained as a yellow solid.

¹H NMR (400 MHz, DMSO-d₆) δ ppm 8.47-8.57 (m, 1H) 8.17 (s, 1H) 7.79-7.85 (m, 1H) 7.74 (s, 1H) 7.68 (s, 1H) 7.65 (s, 1H) 7.50 (d, J=1.88 Hz, 1H) 7.18 (t, J=7.19 Hz, 1H) 7.08 (s, 1H) 7.06 (s, 1H) 6.53 (t, J=7.63 Hz, 1H) 6.42 (d, J=8.63 Hz, 1H) 5.10-5.21 (m, 1H) 3.97 (br d, J=12.26 Hz, 1H) 3.84 (br d, J=13.76 Hz, 1H) 2.73-2.85 (m, 3H) 2.35 (s, 3H) 2.31 (s, 6H) 2.14 (s, 3H) 1.90-1.98 (m, 1H) 1.75-1.82 (m, 1H) 1.59 (d, J=6.63 Hz, 3H) 1.48-1.56 (m, 1H) 1.37-1.47 (m, 1H)

MS m/z (ESI): 540 [M+H]⁺.

### Example 169

### 2-(((R)-1-(2-(4-((S)-3-(dimethylamino)pyrrolidin-1-yl)phenyl)-3,6-dimethyl-4-oxo-4H-chromen-8-yl)ethyl)amino)benzoic acid

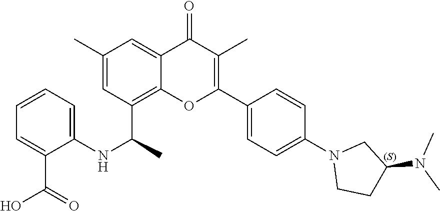

2-(((R)-1-(2-(4-((S)-3-(dimethylamino)pyrrolidin-yl)phenyl)-3,6-dimethyl-4-oxo-4H-chromen-8-yl)ethyl)amino)benzoic acid is prepared in accordance with the method of Example 168. ¹H NMR (400 MHz, DMSO-d₆) δ ppm 8.39 (br d, J=6.25 Hz, 1H) 7.81 (dd, J=8.00, 1.50 Hz, 1H) 7.74 (d, J=1.13 Hz, 1H) 7.67 (d, J=8.76 Hz, 2H) 7.48 (d, J=2.00 Hz, 1H) 7.17-727 (m, 1H) 6.71 (d, J=8.88 Hz, 2H) 6.55 (t, J=7.57 Hz, 1H) 6.44 (d, J=8.51 Hz, 1H) 5.15 (br t, J=6.44 Hz, 1H) 3.60 (br d, J=9.63 Hz, 1H) 3.50 (br s, 2H) 3.17-3.23 (M, 2H) 2.51-2.56 (m, 3H) 2.35 (s, 6H) 2.21-2.27 (m, 1H) 2.14 (s, 3H) 1.92 (br s, 1H) 1.59 (d, J=6.63 Hz, 3H) MS m/z (ESI): 526 [M+H]⁺.

### Example 170

### (R)-2-((1-(2-(4-(3-(dimethylamino)azetidin-1-yl)phenyl)-3,6-dimethyl-4-oxo-411-chromen-8-yl)ethyl)amino)benzoic acid

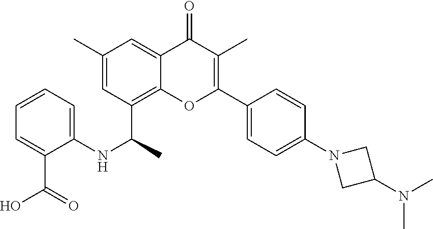 [SMILES: Cc1cc([C@@H](C)Nc2ccccc2C(=O)O)c2oc(-c3ccc(N4CC(N(C)C)C4)cc3)c(C)c(=O)c2c1]

(R)-2-((1-(2-(4-(3-(dimethylamino)azetidin-1-yl)phenyl)-3,6-dimethyl-4-oxo-4H-chromen-8-yl)ethyl)amino)benzoic acid is prepared in accordance with the method of Example 168.

¹H NMR (400 MHz, DMSO-d₆) δ ppm 8.37 (br d, J=5.25 Hz, 1H) 8.13 (s, 1H) 7.80 (dd, J=7.94, 1.56 Hz, 1H) 7.73 (d, J=1.25 Hz, 1H) 7.64 (d, J=8.75 Hz, 2H) 7.48 (d, J=2.00 Hz, 1H) 7.15-7.24 (m, 1H) 6.51-6.60 (m, 3H) 6.43 (d, J==8.25 Hz, 1H) 5.08-5.19 (m, 1H) 3.99 (t, J=7.38 Hz, 2H) 3.69 (dd, J=7.88, 5.38 Hz, 2H) 2.63-2.71 (M, 1H) 2.34 (s, 3H) 2.13 (s, 6H) 2.11 (s, 3H) 1.58 (d, J=6.63 Hz, 3H).

MS m/z (ESI): 512 [M+H]⁺.

### Example 171

### (R)-2-((1-(3,6-dimethyl-2-(4-((1-methylazetidin-3-yl)oxy)phenyl)-4-oxo-4H-chromen-8-yl)ethyl)amino)benzoic acid

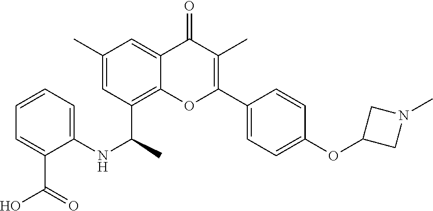 [SMILES: Cc1cc([C@@H](C)Nc2ccccc2C(=O)O)c2oc(-c3ccc(OC4CN(C)C4)cc3)c(C)c(=O)c2c1]

(R)-2-((1-(3,6-dimethyl-2-(4-((1-methylazetidin-3-yl)oxy)phenyl)-4-oxo-4H-chromen-8-yl)ethyl)amino)benzoic acid is prepared in accordance with the method of Example 150.

¹H NMR (400 MHz, DMSO-d6) δ ppm 8.54 (s, 1H) 8.49 (br s, 1H) 8.16 (s, 1H) 7.88 (s, 1H) 7.77 (dd, J=13.51, 8.50 Hz, 2H) 7.51-7.64 (m, 2H) 7.15 (brt, J=7.94 Hz, 1H) 6.49 (t, J=7.38 Hz, 1H) 6.42 (br d, J=8.25 Hz, 1H) 5.50 (br s, 1H) 4.23 (s, 3H) 3.47 (s, 3H) 2.39 (s, 3H) 1.55 (br d, J=6.50 Hz, 3H) MS m/z (ESI): 499 [M+H]⁺.

### Example 172

### 2-(((1R)-1-(3,6-dimethyl-2-(4-((1-methylpyrrolidin-2-yl)methoxy)phenyl)-4-oxo-4H-chromen-8-yl)ethyl)amino)benzoic acid

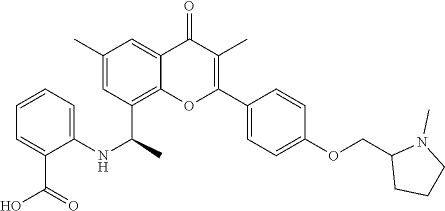 [SMILES: Cc1cc([C@@H](C)Nc2ccccc2C(=O)O)c2oc(-c3ccc(OCC4CCCN4C)cc3)c(C)c(=O)c2c1]

2-[[(1R)-1-[3,6-dimethyl-2-[4-[[(2R)-1-methylpyrrolidin-2-yl]methoxy]phenyl]-4-oxo-chromen-8-yl]ethyl]amino]benzoic acid and 2-[[(1R)-1-[3,6-dimethyl-2-[4-[[(2S)-1-methylpyrrolidin-2-yl]methoxy]phenyl]-4-oxo-chromen-8-yl] ethyl] amino] benzoic acid

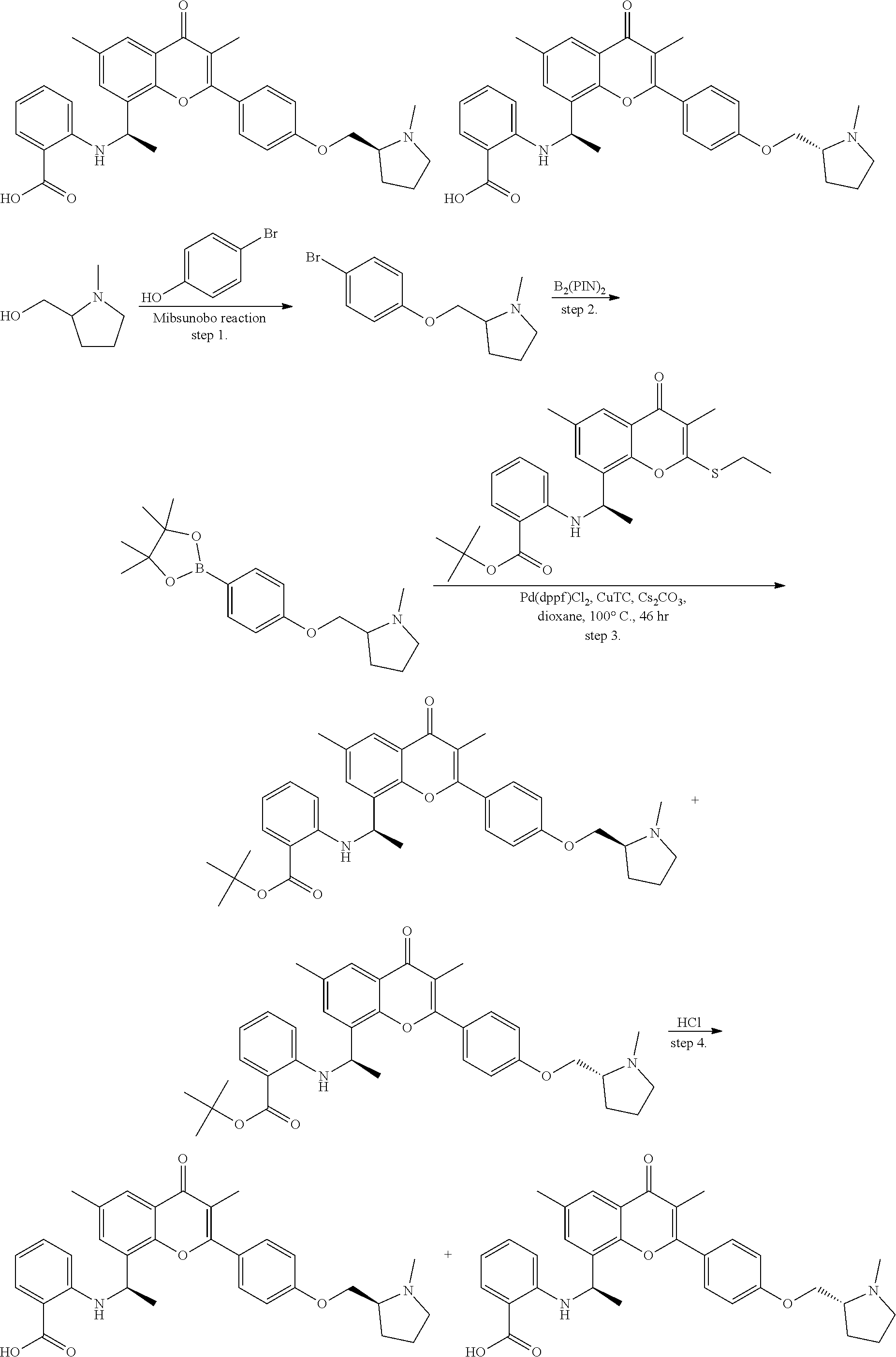

### Step 1

To a solution of (1-methylpyrrolidin-2-yl)methanol (800 mg, 6.95 mmol, 1 eq) and 4-bromophenol (1 g, 8.34 mmol, 1.2 eq) in THF (8 mL) was added PPh₃ (3 g, 13.89 mmol, 2 eq) and DIAD (2 g, 13.89 mmol, 2.70 mL, 2 eq). The mixture was stirred at 25° C. for 16 hr. LCMS showed desired MS was detected. The reaction mixture was filtered to remove the insoluble and concentrated under vacuum to give the crude product. The crude product was purified by flash silica gel chromatography (ISCO®1′; 80 g SepaFlash® Silica Flash Column, Eluent of 0-30% Ethyl acetate/Petroleum ether gradient @ 35 mL/min). Compound 2-[(4-bromophenoxy) methyl]-1-methyl-pyrrolidine (900 mg, 3.33 mmol, 48% yield) was obtained as a black solid.

MS m/z (ES1): 270 [M+H]⁺.

### Step 2

A mixture of 2-[(4-bromophenoxy)methyl]-1-methyl-pyrrolidine (460 mg, 1.70 mmol, 1 eq), 4,4,5,5-tetramethyl-2-(4,4,5,5-tetramethyl-1,3,2-dioxaborolan-2-yl)-1,3,2-dioxaborolane (864 mg, 3.41 mmol, 2 eq), cyclopentyl (diphenyl) phosphane; dichloromethane; dichloropalladium, iron (139 mg, 170.27 umol, 0.1 eq), KOAc (501 mg, 5.11 mmol, 3 eq) in dioxane (5 mL) was degassed and purged with N₂ for 3 times, and then the mixture was stirred at 80° C. for 2 hr under N₂ atmosphere. LCMS showed desired MS was detected. The reaction mixture was added H₂O (10 mL) and extracted with ethyl acetate (10 mL*3). The combined organic layers were dried over anhydrous Na₂SO₄, filtered and concentrated under reduced pressure to give a crude product. The crude product was purified by flash silica gel chromatography (ISCO®; 20 g SepaFlash® Silica Flash Column, Eluent of 0-5% Methanol/Dichloromethane @ 25 mL/min). Compound 1-methyl-2-[[4-(4, 4, 5, 5-tetramethyl-1, 3, 2-dioxaborolan-2-yl) phenoxy] methyl] pyrrolidine (300 mg, 945.69 umol, 55% yield) was obtained as a black solid.

MS m/z (ESI): 317 [M+H]⁺.

### Step 3

A mixture of tert-butyl 2-[[(1R)-1-(2-ethylsulfanyl-3,6-dimethyl-4-oxo-chromen-8-yl)ethyl]amino]benzoate (50 mg, 110.23 umol, 1 eq), 1-methyl-2-[[4-(4,4,5,5-tetramethyl-1,3,2-dioxaborolan-2-yl)phenoxy]methyl]pyrrolidine (69 mg, 220.46 umol, 2 eq), thiophene-2-carbonyloxycopper (42 mg, 220.46 umol, 2 eq), cyclopentyl (diphenyl)phosphane; dichloromethane; dichlioropalladium; iron (18 mg, 22.05 umol, 0.2 eq) and Cs₂CO₃ (71 mg, 220.46 umol, 2 eq) in dioxane (3 mL) was degassed and purged with N₂ for 3 times, and then the mixture was stirred at 50° C. for 16 hr under N₂ atmosphere. LCMS showed desired MS was detected. The reaction mixture was filtered to remove the insoluble and concentrated under vacuum to give the crude product. The residue was purified by prep-HPLC (column: Welch Xtimate C18 150*30 mm*5 um; mobile phase: [water (FA)-ACN]; B %: 14%-54%, 25 min) to give desired compound as a white solid, which was further separated by SFC (column: DAICEL CHIRALCEL OX (250 mm*30 mm, 1 0 um); mobile phase: [0.1% NH3H2O ETOH]; 35% B isocratic elution mode). Compound 2-[[(1R)-1-[6-methyl-2-[2-(2-morpholinoethyl)indazol-5-yl]-4-oxo-chromen-8-yl]ethyl] amino] benzoic acid (7 mg, 11.16 umol, 11% yield, 88% purity) and compound 2-[[(l S)-1-[6-methyl-2-[2-(2-morpholinoethyl)indazol-5-yl]-4-oxo-chromen-8-yl] ethyl] amino] benzoic acid (6 mg, 9.71 umol, 10% yield, 89% purity) were obtained as two white solids. (Peak 1: t1/2=1.37 min, A; Peak 2: t1/2=1.51 min, B)

MS m/z (ESI): 583 [M+H]⁺.

### Step 4

To a solution of tert-butyl 2-[[(1R)-1-[3,6-dimethyl-2-[4-[[(2S)-1-methyl pyrrolidin-2-yl]methoxy]phenyl]-4-oxo-chromen-8-yl]ethyl]amino]benzoate or -[[(1R)-1-[3, 6-dimethyl-2-[4-[[(2R)-1-methyl pyrrolidin-2-yl] methoxy] phenyl]-4-oxo-chromen-8-yl] ethyl] amino] (6 mg, 10.30 umol, 1 eq) in ACN (1 mL) was added HCl(aq.) (12 M, 3.43 uL, 4 eq). The mixture was stirred at 80° C. for 1 hr. LCMS showed desired MS was detected. The reaction mixture was filtered to remove the insoluble and concentrated under vacuum to give the crude product. The residue was purified by prep-HPLC (column: Welch Xtimate C18 150*30 mm*5 um; mobile phase: [water (FA)-MeOH]; B %: 4%-44%, 25 min).

172A Peak 1,

¹H NMR (400 MHz, METHANOL-d₄) δ ppm 7.91 (br d, J=7.25 Hz, 1H) 7.85 (s, 1H) 7.71-7.78 (m, 2H) 7.64 (s, 1H) 7.17-7.24 (m, 2H) 7.08 (br t, J=7.57 Hz, 1H) 6.53 (br t, J=⁷0.44 Hz, 1H) 6.37 (d, J=8.63 Hz, 1H) 5.17 (br s, 1H) 3.10-3.23 (n, 2H) 2.87-2.98 (m, 2H) 2.67 (s, 3H) 2.40 (s, 3H) 2.17-2.21 (m, 3H) 1.87-2.09 (m, 4H) 1.77-1.83 (m, 1H) 1.64 (brd, J=6.38 Hz, 3H)

MS m/z (ESI): 527 [M+H]⁺.

172B Peak 2,

¹H NMR (400 MHz, DMSO-d₆) δ ppm 7.88-7.95 (m, 1H) 7.85 (s, 1H) 7.73 (br d, J=7.63 Hz, 2H) 7.64 (s, 1H) 7.19 (br d, J=8.50 Hz, 2H) 704-7.12 (m, 1H) 6.53 (brt, J=7.00 Hz, 1H) 6.37 (br d, J=8.38 Hz, 1H) 5.16 (br d, J=6.63 Hz, 1H) 3.15-3.26 (m, 2H) 2.95 (br d, J=2.75 Hz, 2H) 2.69 (br s, 3H) 2.40 (s, 3H) 2.19 (d, J=2.88 Hz, 3H) 1.88-2.09 (m, 4H) 1.81 (d, J=9.16, 4.44 Hz, 1H) 1.63 (br d, J=5.75 Hz, 3H)

MS m/z (ESI): 527 [M+H]⁺.

### Example 176

### ((2-((1-(3,6-dimethyl-2-(2-methyl-2H-indazol-5-yl)-4-oxo-4H-chromen-8-yl)ethyl)amino)phenyl)phosphonic acid

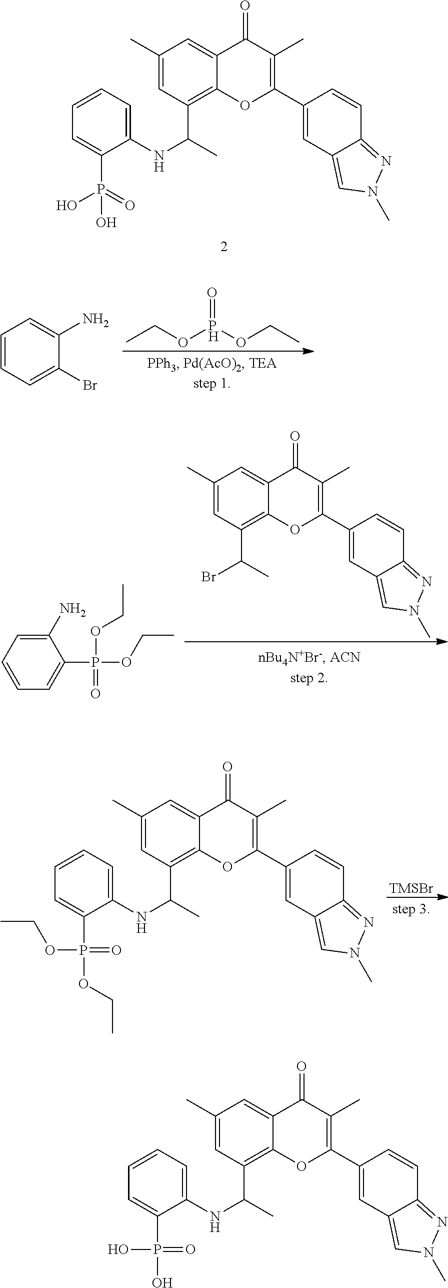

### Step 1

To a solution of 2-bromoaniline (2 g, 11.63 mmol, 1 eq) and 1-ethoxyphosphinyl oxyethane (1.93 g, 13.95 mmol, 1.80 mL, 1.2 eq) in EtOH (20 mL) was added PPh₃ (457.4 mg, 1.74 mmol, 0.15 eq), TEA (1.76 g, 17.44 mmol, 2.43 mL, 1.5 eq) and Pd(OAc)₂ (130.5 mg, 581.32 umol, 0.05 eq). The mixture was stirred at 80° C. for 12 hr under N₂ atmosphere. LCMS showed desired mass was detected. The reaction mixture was diluted with EtOAc (50 mL) and filtered to remove the insoluble and concentrated under vacuum to give the crude product. The residue was purified by flash silica gel chromatography (ISCO®; 40 g SepaFlash® Silica Flash Column, Eluent of 0-30% Ethyl acetate/Petroleum ether gradient @ 40 mL/min). Compound 2-diethoxyphosphorylniline (800 mg, 3.49 mmol, 30% yield) was obtained as a yellow oil.

MS m/z (ESI: 230 [M+H]⁺.

### Step 2

To a solution of 8-(1-bromoethyl)-3, 6-dimethyl-2-(2-methylindazol-5-yl) chromen-4-one (300 mg, 729.41 umol, 1 eq), 2-diethoxyphosphoryl niline (200.6 mg, 875.29 umol, 1.2 eq) in ACN (3 mL) was added TBAB (587.8 mg, 1.82 mmol, 2.5 eq). The mixture was stirred at 90° C. for 12 hr. LCMS showed desired mass was detected. The reaction mixture concentrated under vacuum to give the crude product. The residue was purified by prep-HPLC (column: Welch Xtimate C18 150*30 mm*Sum; mobile phase: [water (FA)-ACN]; B %: 34%-74%, 25 min). Compound 8-[1-(2-diethoxyphosphorylanilino)ethyl]-3,6-dimethyl-2-(2-methylindazol-5-yl)chromen-4-one (40 mg, 70.07 umol, 10% yield, 98% purity) was obtained as a white solid.

MS m/z (ESI): 560[M+H]⁺.

### Step 3

To a solution of 8-[1-(2-diethoxyphosphorylanilino)ethyl]-3,6-dimethyl-2-(2-methyl indazol-5-yl)chromen-4-one (30 mg, 53.61 umol, 1 eq) in DCM (1 mL) was added TMSBr (82.1 mg, 536.11 umol, 69.55 uL, 10 eq) and 2,6-dimethylpyridine (20.1 mg, 187.64 umol, 21.85 uL, 3.5 eq). The mixture was stirred at 25° C. for 2 hr. LCMS showed desired MS was detected. The reaction mixture was filtered and concentrated under reduced pressure to give the crude product. The residue was purified by prep-HPLC (column: Welch Xtimate C18 150*30 mm*5 um; mobile phase: [water (NH₄CO₃)-ACN]; B %: 0%-34%, 28 min). Compound [2-[1-[3, 6-dimethyl-2-(2-methylindazol-5-yl)-4-oxo-chromen-8-yl] ethylamino] phenyl] phosphonic acid (17.8 mg, 32.87 umol, 61% yield, 93% purity) was obtained as a white solid.

¹H NMR (400 MHz, DMSO-d₆) δ ppm 8.55 (s, 1H) 8.24 (s, 1H) 7.69-7.83 (m, 2H) 7.58-7.68 (m, 2H) 7.46 (br dd, J=14.20, 7.94 Hz, 1H) 7.04-7.36 (m, 2H) 6.87-7.01 (m, 1H) 6.42 (br t, J=6.69 Hz, 1H) 6.20 (br t, J=6.50 Hz, 1H) 5.03 (br d, J=6.50 Hz, 1H) 4.22 (s, 3H) 2.33 (s, ³H) 2.14 (s, 3H) 1.49 (br d, J=6.50 Hz, 3H) MS m/z (ESI): 504 [M+H]⁺.

### Example 177

### 3-(2-((1-(3,6-dimethyl-2-(2-methyl-2H-indazol-5-yl)-4-oxo-4H-chromen-8-yl)ethyl)amino)phenyl)-1,2,4-oxadiazol-5(2H)-one

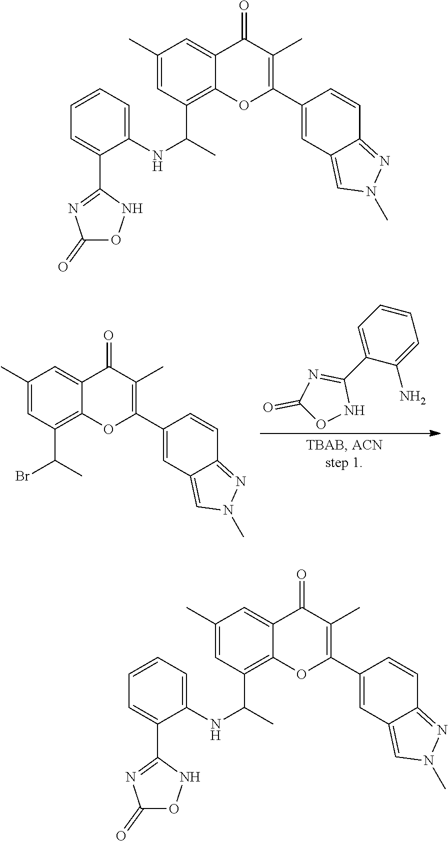

### Step 1

To a solution of 3-(2-aminophenyl)-2H-1,2,4-oxadiazol-5-one (31.0 mg, 175.06 umol, 1.2 eq) (prepared in accordance with the method in patent WO2005/56532) and 8-(1-bromoethyl)-3,6-dimethyl-2-(2-methylindazol-5-yl)chromen-4-one (60 mg, 145.88 umol, 1 eq) in ACN (2 mL) was added TBAB (117.5 mg, 364.71 umol, 2.5 eq). The mixture was stirred at 80° C. for 16 hr. The reaction mixture was concentrated under vacuum to give the crude product. The crude product was purified by prep-HPLC (column: Xtimate C18 100*30 mm*10 um; mobile phase: [water (FA)-ACN]; B %: 28%-68%, 25 min). Compound 3-[2-[1-[3,6-dimethyl-2-(2-methylindazol-5-yl)-4-oxo-chromen-8-yl]ethylamino]phenyl]-2H-1,2,4-oxadiazol-5-one (13 mg, 25.09 umol, 17% yield, 97% purity) was obtained as a white solid.

¹H NMR (400 MHz, ACETONITRILE-d₃) δ ppm 12.82 (br d, J=5.75 Hz, 1H) 8.53 (s, 1H) 8.20 (s, 1H) 7.71-7.82 (m, 2H) 7.52-7.66 (m, 3H) 7.22 (s, 1H) 7.05 (br d, J=6.13 Hz, 1H) 6.70 (s, 1H) 6.57 (d, J=8.50 Hz, 1H) 5.20 (br t, J=6.32 Hz, 1H) 4.23 (s, 3H) 2.36 (s, 3H) 2.13 (s, 3H) 1.62 (d, J=6.63 Hz, 3H)

MS m/z (ES1): 508 [M+H]⁺.

### Example 178

### N-((2-((1-(3,6-dimethyl-2-(2-methyl-2H-indazol-5-yl)-4-oxo-4H-chromen-8-yl)ethyl)amino)phenyl)sulfonyl)acetamide

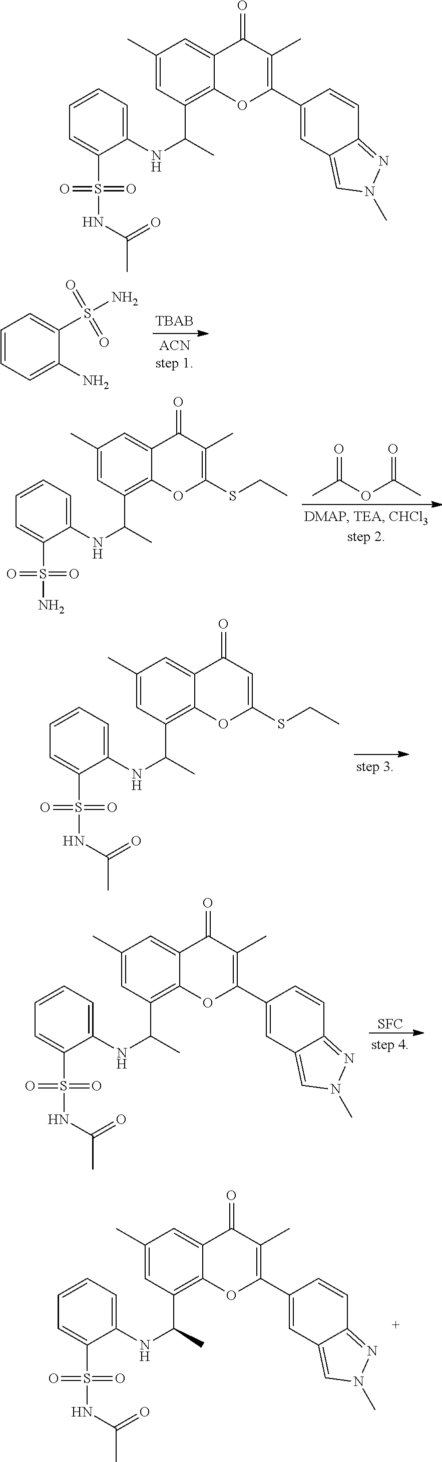

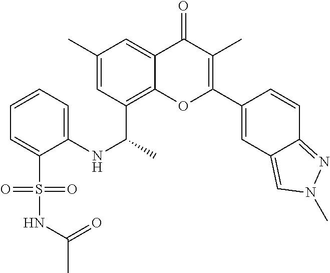 [SMILES: CC(=O)NS(=O)(=O)c1ccccc1N[C@@H](C)c1cc(C)cc2c(=O)c(C)c(-c3ccc4nn(C)cc4c3)oc12]

### Step 1

To a solution of 8-(1-bromoethyl)-2-ethylsulfanyl-3,6-dimethyl-chromen-4-one (310 mg, 908.39 umol, 1 eq), 2-aminobenzenesulfonamide (187.72 mg, 1.09 mmol, 1.2 eq) in ACN (5 ML) was added TBAB (732 mg, 2.27 mmol, 2.5 eq). The mixture was stirred at 90° C. for 12 hr. The reaction mixture was extracted with ethyl acetate (30 ml*3). The combined organic layers were dried over anhydrous Na₂SO₄, filtered and concentrated under reduced pressure to give a crude product. The residue was purified by flash silica gel chromatography (ISCO®; 12 g SepaFlash® Silica Flash Column, Eluent of 0-30% Ethyl acetate/Petroleum ether gradient @ 40 mL/min). Compound 2-[1-(2-ethylsulfanyl-3, 6-dimethyl-4-oxo-chromen-8-yl) ethylamino]benzene sulfonamide (280 mg, 647.32 umol, 71% yield, N/A purity) was obtained as a white solid. MS m/z (ESI): 433 [M+H]⁺.

### Step 2

To a solution of 2-[1-(2-ethylsulfanyl-3,6-dimethyl-4-oxo-chromen-8-yl)ethylamino]benzenesulfonamide (260 mg, 601.08 umol, 1 eq), acetyl acetate (73 mg, 721.30 umol, 67.56 uL, 1.2 eq) in CHCl₃ (5 mL) was added DMAP (73 mg, 601.08 umol, 1 eq) and TEA (182 mg, 1.80 mmol, 250.99 uL, 3 eq). The mixture was stirred at 25° C. for 3 hr. The reaction mixture was extracted with dichloromethane (10 ml*3). The combined organic layers were dried over anhydrous Na₂SO₄, filtered and concentrated under reduced pressure to give a crude product. The residue was purified by flash silica gel chromatography (ISCO®; 4 g SepaiFlash®Silica Flash Column, Eluent of 0˜60% Ethyl acetate/Petroleum ether gradient@ 40 mL/min). Compound N-[2-[1-(2-ethylsulfanyl-3,6-dimethyl-4-oxo-chromen-8-yl)ethylamino]phenyl]sulfanylacetamide (250 mg, 474.09 umol, 79% yield, 90% purity) was obtained as a white solid. MS m/z (ESI): 475 [M+H]

### Step 3

A mixture of N-[2-[1-(2-ethylsulfanyl-3,6-dimethyl-4-oxo-chromen-8-yl)ethylamino]phenyl]sulfanylacetamide (200 mg, 421.41 umol, 1 eq), 2-methyl-5-(4,4,5,5-tetramethyl-1,3,2-dioxaborolan-2-yl)indazole (217 mg, 842.83 umol, 2 eq), Cs₂CO₃ (274 mg, 842.83 umol, 2 eq), Pd(dppf)Cl₂·CH₂Cl₂ (68 mg, 84.28 umol, 0.2 eq) and thiophene-2-carbonyl oxycopper (160 mg, 842.83 umol, 2 eq) in dioxane (4 mL) was degassed and purged with N₂ for 3 times, and then the mixture was stirred at 80° C. for 16 hr under N₂ atmosphere. The reaction mixture was extracted with dichloromethane (20 ml*3). The combined organic layers were dried over anhydrous Na₂SO₄, filtered and concentrated under reduced pressure to give a crude product. The residue was purified by prep-HPLC (column: Welch Xtimate C18 150*30 mm*5 um; mobile phase: [water (FA)-ACN]; B %: 24%-64%, 25 min). Compound N-[2-[1-[3, 6-dimethyl-2-(2-methylindazol-5-yl)-4-oxo-chromen-8-yl] ethylamino] phenyl] sulfanylacetamide (30 mg, 53.98 umol, 13% yield, 98% purity) was obtained as a white solid.

MS m/z (ESI): 545 [M+H]⁺.

### Step 4

The stereoisomers of example N-[2-[[(R)-1-[3,6-dimethyl-2-(2-methylindazol-5-yl)-4-oxo-chromen-8-yl]ethyl]amino]phenyl]sulfonylacetamide and N-[2-[[(1R)-1-[3,6-dimethyl-2-(2-methyl indazol-5-yl)-4-oxo-chromen-8-yl]ethyl]amino]phenyl]sulfanylacetamide.

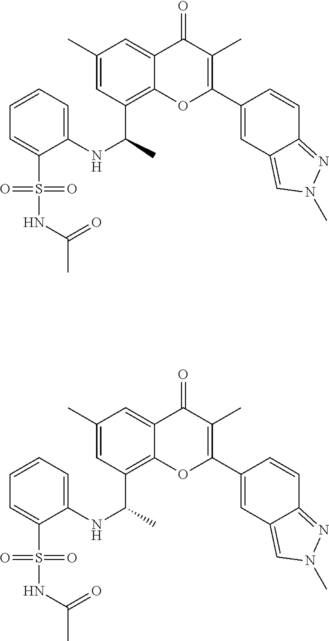 [SMILES: CC(=O)NS(=O)(=O)c1ccccc1N[C@H](C)c1cc(C)cc2c(=O)c(C)c(-c3ccc4nn(C)cc4c3)oc12 ; CC(=O)NS(=O)(=O)c1ccccc1N[C@@H](C)c1cc(C)cc2c(=O)c(C)c(-c3ccc4nn(C)cc4c3)oc12]

Enantiomeric separation of N-((2-((1-(3,6-dimethyl-2-(2-methyl-2H-indazol-5-yl)-4-oxo-4H-chromen-8-yl)ethyl)amino)phenyl)sulfonoyl)acetamide were prepared in accordance with the method of Example 20 by chiral SFC (column: ChiralPak IH, 250*30 mm, 10 um; mobile phase: [0.1% NH₃H₂O ETOH]; B %: 40%-40%, min) to give Peak 1 (Rt=2.69 min, 178A) and Peak 2 (Rt==2.79 min, 178B). 178A and 17813 were assigned arbitrarily.

### Peak 1.

¹H NMR (400 MHz, DMSO-d₆) δ ppm 8.54 (s, 1H) 8.24 (s, 1H) 7.72-7.79 (m, 2H) 7.62-7.67 (n, 2H) 7.58 (d, J=8.00 Hz, 1H) 70.05 (br t, J=7.75 Hz, 1H) 6.91 (br d, J=5.13 Hz, 1H) 6.53 (t, J=7.50 Hz, 1H) 6.32 (br d, J=8.38 Hz, 1H) 5.11 (br t, J=6.63 Hz, 1H) 4.23 (s, 3H) 2.35 (s, 3H) 2.15 (s, 3H) 1.77 (s, 3H) 1.55 (br d, J=6.63 Hz, 3H) MS m/z (ESI: 545 [M+H]⁺.

### Peak 2.

¹H NMR (400 MHz, DMSO-d₆) δ ppm 8.54 (s, 1H) 8.24 (s, 1H) 7.75 (br d, J=3.38 Hz, 2H) 7.62-7.68 (m, 2H) 7.59 (br d, J=7.38 Hz, 1H) 7.01-7.07 (m, 1H) 6.92 (br d, J=5.25 Hz, 1H) 6.53 (br t, J=7.25 Hz, 1H) 6.32 (br d, J=8.13 Hz, 1H) 5.11 (br t, J=6.50 Hz, 1H) 4.23 (s, 3H) 2.35 (s, 3H) 2.14 (s, 3H) 1.77 (s, 3H) 1.55 (br d, J=6.25 Hz, 3H)

MS m/z (ESI): 545 [M+H]⁺.

### Example 179

### (R)-2-((1-(2-(4-(4-acetylpiperazin-1-yl)phenyl)-6-chloro-4-oxo-41H-chromen-8-yl)ethyl)amino)benzoic acid

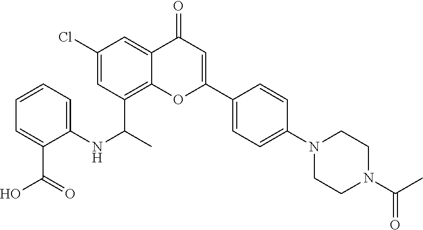 [SMILES: CC(=O)N1CCN(c2ccc(-c3cc(=O)c4cc(Cl)cc(C(C)Nc5ccccc5C(=O)O)c4o3)cc2)CC1]

R)-2-((1-(2-(4-(4-acetylpiperazin-1-yl)phenyl)-6-chloro-4-oxo-4H-chromen-8-yl)ethyl)amino)benzoic acid is prepared in accordance with the method of Example 98.

¹H NMR (400 MHz, DMSO-d₆) δ ppm 8.81 (br s, 1H) 8.07 (br d, J=9.01 Hz, 2H) 7.84-7.96 (n 2H) 7.69 (d, J=2.25 Hz, 1H) 7.20-7.29 (m, 1H) 7.16 (brd, J=9.01 Hz, 2H) 7.01-7.08 (m, 1H) 6.62 (s, 1H) 6.51 (br d, J=8.50 Hz, 1H) 5.33-5.43 (m, 1H) 3.60-3.69 (m, 4H) 3.47 (br d, J=5.38 Hz, 4H) 2.11 (s, 3H) 1.74 (br d, J=6.50 Hz, 3H)

MS m/z (ESI): 481 [M+H]⁺.

### Example 182

### 2-((1-(6-methyl-2-(4-(4-methyl-3-oxopiperazin-1-yl)phenyl)-4-oxo-4H-chromen-8-yl)ethyl)amino)benzoic acid

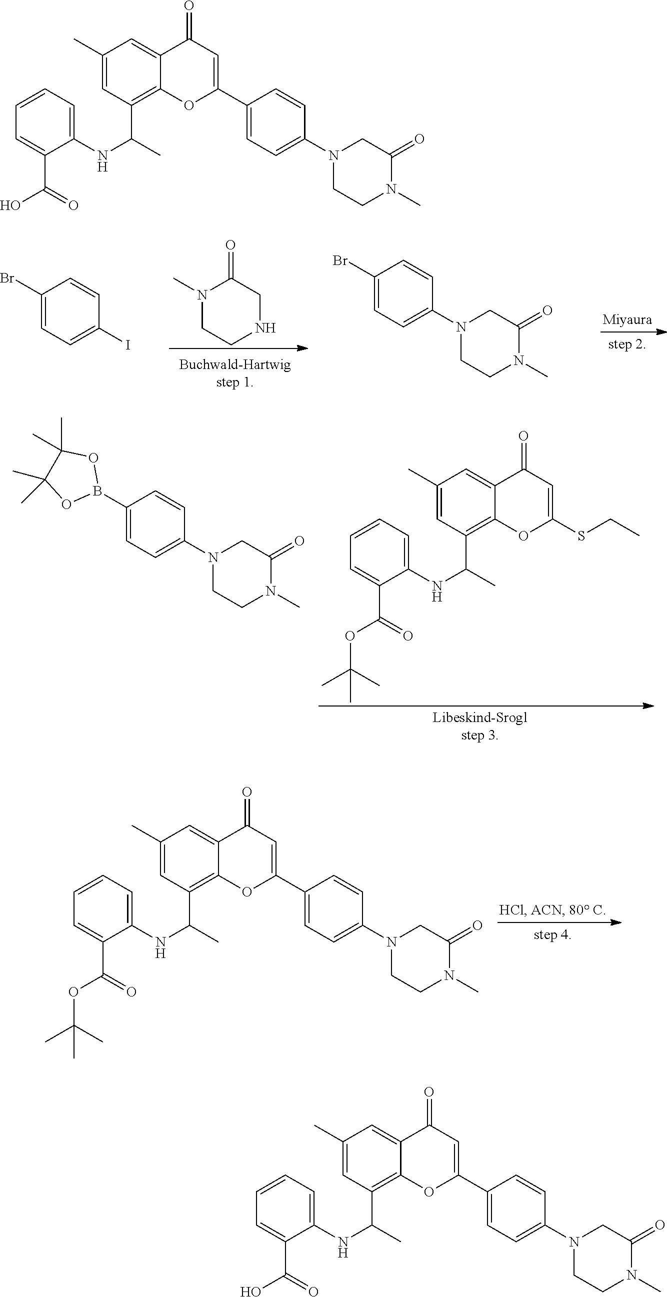

### Step 1

To a solution of 1-bromo-4-iodo-benzene (1 g, 3.53 mmol, 1 eq), 1-methylpiperazin-2-one (404 mg, 3.53 mmol, 1 eq), Cs₂CO₃ (2.30 g, 7.07 mmol, 2 eq), Xantphos (205 mg, 353.48 umol, 0.1 eq) and Pd₂(dba)₃ (324 mg, 353.48 umol, 0.1 eq) in dioxane (20 mL) was degassed and purged with N₂ for 3 times, and then the mixture was stirred at 80° C. for 16 hr under N₂ atmosphere. The reaction mixture was diluted with H₂O (100 mL) and extracted with ethyl acetate (150 mL*3). The combined organic layers were dried over anhydrous Na₂SO₄, filtered and concentrated under reduced pressure to give a crude product. The crude product was purified by flash silica gel chromatography (ISCO®; 12 g SepaFlash® Silica Flash Column, Eluent of 0˜100% Ethyl acetate/Petroleum ether gradient @ 40 mL/min). Compound 4-(4-bromophenyl)-1-methyl-piperazin-2-one (300 mg, 1.11 mmol, 32% yield) was obtained as a yellow solid.

MS m/z (ESI): 269 [M+H]⁺.

### Step 2

4-(4-bromophenyl)-1-methyl-piperazin-2-one (300 mg, 1.11 mmol, 1 eq), 4,4,5,5-tetramethyl-2-(4,4,5,5-tetramethyl-1,3,2-dioxaborolan-2-yl)-1,3,2-dioxaborolane (425 mg, 1.67 mmol, 1.5 eq), KOAc (328.18 mg, 3.34 mmol, 3 eq), XPhos (53 mg, 111.47 umol, 0.1 eq) and Pd₂(dba)₃ (102 mg, 111.47 umol, 0.1 eq) in dioxane (7 mL) was degassed and purged with N₂ for 3 times, and then the mixture was stirred at 80° C. for 16 hr under N₂ atmosphere. The reaction mixture was diluted with H₂O (50 mL) and extracted with ethyl acetate (50 mL*3). The combined organic layers were dried over anhydrous Na₂SO₄, filtered and concentrated under reduced pressure to give a crude product. The crude product was purified by flash silica gel chromatography (ISCO®; 4 g SepaFlash® Silica Flash Column, Eluent of 0-100% Ethyl acetate/Petroleum ether gradient @18 mL/min). Compound 1-methyl-4-[4-(4,4,5,5-tetramethyl-1,3,2-dioxaborolan-2-yl)phenyl]piperazin-2-one (220 mg, 695.76 umol, 62% yield) was obtained as a yellow solid. MS m/z (ESI): 317 [M+H]⁺.

### Step 3

To a solution of tert-butyl 2-[1-(2-ethylsulfanyl-6-methyl-4-oxo-chromen-8-yl) ethylamino]benzoate (40 mg, 91.00 umol, 1 eq), 1-methyl-4-[4-(4,4,5,5-tetramethyl-1,3,2-dioxaborolan-2-yl)phenyl]piperazin-2-one (86 mg, 273.00 umol, 3 eq), Cs₂CO₃ (59 mg. 182.00 umol, 2 eq), thiophene-2-carbonyloxycopper (35 mg, 182.00 umol, 2 eq) and Pd(dppf)Cl₂·CH₂Cl₂ (15 mg, 18.20 umol, 0.2 eq) in dioxane (1.5 mL) was degassed and purged with N₂ for 3 times, and then the mixture was stirred at 100° C. for 16 hr under N₂ atmosphere. The reaction mixture was diluted with 1120 (50 mL) and extracted with ethyl acetate (50 mL*3). The combined organic layers were dried over anhydrous Na₂SO₄, filtered and concentrated under reduced pressure to give a crude product. The crude product was purified by flash silica gel chromatography (ISCOR; 4 g SepaFlash® Silica Flash Column, Eluent of 0˜50% methanol/dichloromethane @ 18 mL/min). Compound tert-butyl 2-[1-[6-methyl-2-[4-(4-methyl-3-oxo-piperazin-1-yl)phenyl]-4-oxo-chromen-8-yl]ethylamino]benzoate (40 mg, crude) was obtained as a black solid.

MS m/z (ESI): 568 [M+H]⁺.

### Step 4

To a solution of tert-butyl 2-[1-R[6-methyl-2-[4-(4-methyl-3-oxo-piperazin-1-yl)phenyl]-4-oxo-chromen-8-yl]ethylamino]benzoate (40 mg, 70.46 umol, 1 eq) in MeCN (1 mL) was added HCl (12 M, 35 μL, 6 eq), and the mixture was stirred at 80° C. for 1 hr. The reaction mixture was filtered to remove the insoluble and concentrated under vacuum to give the crude product. The crude product was purified by prep-HPLC (FA condition, column: Welch Xtimate C18 150*30 mm*5 um; mobile phase: [water (F A)-ACN]; B %: 0%-90%, 36 min). Compound 2-[1-[6-methyl-2-[4-(4-methyl-3-oxo-piperazin-1-yl)phenyl]-4-oxo-chromen-8-yl]ethylamino]benzoic acid (6.0 mg, 10.71 umol, 15% yield, 91% purity) was obtained as a yellow solid.

¹H NMR (400 MHz, DMSO-d₆) δ ppm 8.44 (br d, J=5.88 Hz, 1H) 7.99 (br d, J=8.76 Hz, 2H) 7.82 (br d, J=7.38 Hz, 1H) 7.72 (s, 1H) 7.52 (s, 1H) 7.23 (br t, J=7.88 Hz, 1H) 7.06 (br d, J=9.01 Hz, 2H) 6.90 (s, 1H) 6.47-6.59 (m, 2H) 5.30 (br t, J=6.69 Hz, 1H) 3.95 (s, 2H) 3.67 (br t, J=5.19 Hz, 2H) 3.47 (br t, J=5.25 Hz, 2H) 2.91 (s, 3H) 2.35 (s, 3H) 1.67 (br d, J=6.38 Hz, 3H)

MS m/z (ESI): 512 [M+H]⁺.

### Example 183

### 5-fluoro-2-((1-(6-methyl-2-(2-methyl-2H-indazol-5-yl)-4-oxo-4H-chromen-8-yl)ethyl)amino)benzoic acid

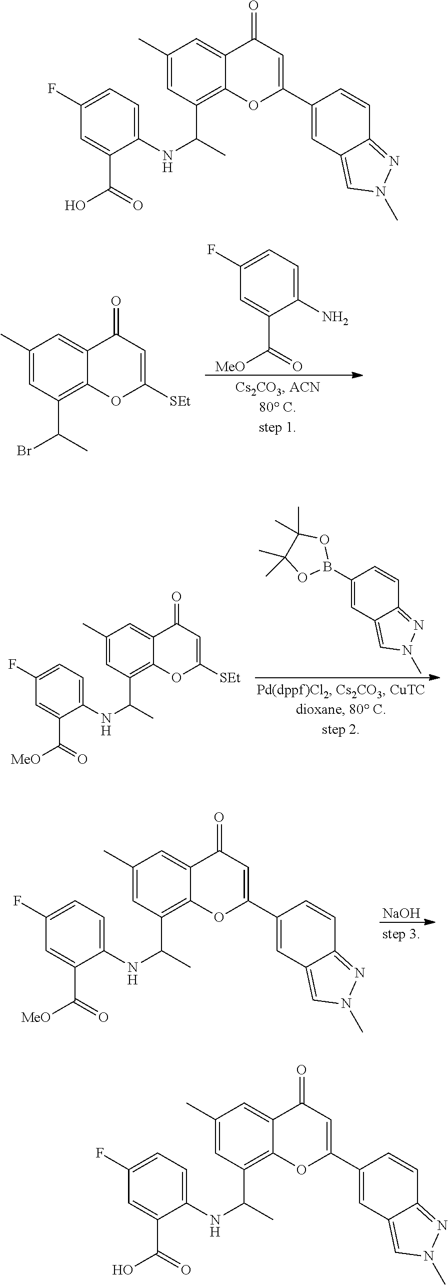

### Step 1

To a solution of 8-(1-bromoethyl)-2-ethylsulfanyl-6-methyl-chromen-4-one (ZXP-3161-1, 100 mg, 305.59 umol) methyl 2-amino-5-fluoro-benzoate (62.03 mg, 366.71 umol) and Cs₂CO₃ (199.13 mg. 611.18 umol) in ACN (2 mL) was degassed and purged with N₂ for 3 times, and then the mixture was stirred at 80° C. for 12 h under N₂ atmosphere. The mixture was concentrated under reduced pressure to give a residue. The residue was purified by prep-TLC (SiO₂, Petroleum ether:Ethyl acetate=4:1) to give methyl 2-((1-(2-(ethylthio)-6-methyl-4-oxo-4H-chromen-8-yl)ethyl)amino)-5-fluorobenzoate (70.0 mg, 161.74 umol, 52.93% yield, 96_00% purity) as a yellow solid.

MS m/z (ESI): 417 [M+H]⁺.

### Step 2

To a solution of methyl 2-((1-(2-(ethylthio)-6-methyl-4-oxo-4H-chromen-8-yl)ethyl)amino)-5-fluorobenzoate (50 mg, 120.34 umol), 2-methyl-5-(4,4,5,5-tetramethyl-1,3,2-dioxaborolan-2-yl)-2H-indazole (62.13 mg, 240.69 umol), Pd(dppf)Cl₂ (88.06 mg, 120.34 umol), Cs₂CO₃ (117.63 mg, 361.03 umol) and CuTC (45.90 mg, 240.69 umol) was added dioxane (1 mL) with stirring at 80° C. for 13 hr under N₂. The mixture was concentrated under reduced pressure to give a residue. The residue was purified by prep-TLC (SiO₂, Petroleum ether:Ethyl acetate=1:2) to give methyl 5-fluoro-2-((1-(6-methyl-2-(2-methyl-2H-indazol-5-yl)-4-oxo-4H-chromen-8-yl)ethyl)amino)benzoate (20.0 mg, 37.07 umol, 30.81% yield, 90.00% purity) as a yellow solid.

MS m/z (ESI): 486 [M+H]⁺.

### Step 3

To a solution of methyl 5-fluoro-2-((1-(6-methyl-2-(2-methyl-2H-indazol-5-yl)-4-oxo-4H-chromen-8-yl)ethyl)amino)benzoate (3000 mg, 61.79 umol) in MeOH (0.5 mL) and H₂O (0.5 mL) was added NaOH (7.41 mg, 185.37 umol) at 25° C. The mixture was stirred at 40° C. for 2 h. The reaction mixture was concentrated under reduced pressure and the residue was acidified with 2N HCl to pH=7 to give a residue. The residue was purified by prep-HPLC (column: Phenomenex luna C18 150*25 mm*10 um; mobile phase: [water (FA)-ACN]; 13%: 37%-67%, 10 min) to give 5-fluoro-2-((1-(6-methyl-2-(2-methyl-2H-indazol-5-yl)-4-oxo-4H-chromen-8-yl)ethyl)amino)benzoic acid (6 mg, 12.14 umol, 19.64% yield, 95.37% purity) as a white solid.

¹H NMR (400 MHz, DMSO-d) δ ppm 8.62 (s, 1H), 8.58 (s, 1H), 7.91 (br d, J=9.20 Hz, 1H), 7.71-7.79 (m, 2H), 7.55 (s, 2H), 7.10 (br d, J=8.40 Hz, 1H), 7.05 (s, 1H), 6.47-6.54 (m, 1H), 5.45-5.23 (m, 1H), 4.23 (s, 3H), 2.33-2.39 (m, 3H), 1.69 (d, J=6.40 Hz, 3H).

MS m/z (ESI): 472 [M+H]⁺.

### Example 184

### 2-fluoro-6-((1-(6-methyl-2-(2-methyl-2H-indazol-5-yl)-4-oxo-4H-chromen-8-yl)ethyl)amino)benzoic acid

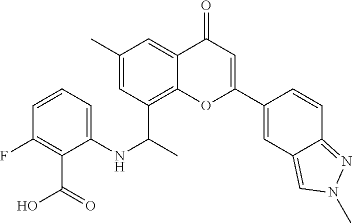 [SMILES: Cc1cc(C(C)Nc2cccc(F)c2C(=O)O)c2oc(-c3ccc4nn(C)cc4c3)cc(=O)c2c1]

2-fluoro-6-((1-(6-methyl-2-(2-methyl-2H-indazol-5-yl)-4-oxo-4H-chromen-8-yl)ethyl)amino)benzoic acid is prepared in accordance with the method of Example 183.

¹H NMR (400 MHz, DMSO-d₆) δ ppm 8.62 (s, 1H) 8.58 (s, 1H) 8.25-8.34 (m, 1H) 7.91 (dd, J=9.26, 1.75 Hz, 1H) 7.71-7.78 (m, 2H) 7.57 (d, J=2.00 Hz, 1H) 7.09-7.16 (m, 1H) 7.06 (s, 1H) 6.28-6.37 (n, 2H) 5.34 (br s, 1H) 4.23 (s, 3H) 2.38 (s, 3H) 1.68 (d, =6.50 Hz, 3H)

MS m/z (ESI): 472 [M+H]⁺.

### Example 185

### 24-fluoro-2-((1-(6-methyl-2-(2-methyl-21H-indazol-5-yl)-4-oxo-4H-chromen-8-yl)ethyl)amino)benzoic acid

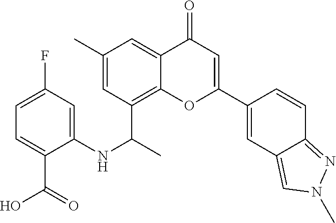 [SMILES: Cc1cc(C(C)Nc2cc(F)ccc2C(=O)O)c2oc(-c3ccc4nn(C)cc4c3)cc(=O)c2c1]

4-fluoro-2-((1-(6-methyl-2-(2-methyl-2H-indazol-5-yl)-4-oxo-4H-chromen-8-yl)ethyl)amino)benzoic acid is prepared in accordance with the method of Example 183.

¹H NMR (400 MHz, DMSO-d₆) 5 ppm 8.62 (s, 1H) 8.58 (s, 1H) 8.25-8.34 (m, 1H) 7.91 (dd, J=9.26, 1.75 Hz, 1H) 7.71-7.78 (m, 2H) 7.57 (d, J=2.00 Hz, 1H) 7.09-7.16 (m, 1H) 7.06 (s, 1H) 6.28-6.37 (n, 2H) 534 (br s, 1H) 4.23 (s, 3H) 2.38 (s, 3H) 1.68 (d, J=6.50 Hz, 3H) MS m/z (ESI: 472 [M+H]⁺.

### Example 186

### (R)-6-chloro-3-((1-(3,6-dimethyl-2-(6-(1-methyl-1H-pyrazol-4-yl)pyridin-3-yl)-4-oxo-411-chromen-8-yl)ethyl)amino)picolinic acid

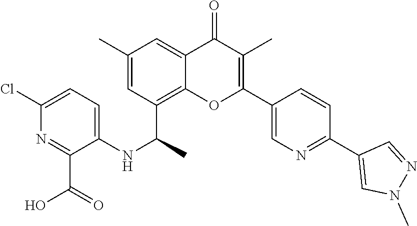 [SMILES: Cc1cc([C@@H](C)Nc2ccc(Cl)nc2C(=O)O)c2oc(-c3ccc(-c4cnn(C)c4)nc3)c(C)c(=O)c2c1]

(R)-6-chloro-3-((1-(3,6-dimethyl-2-(6-(1-methyl-1H-pyrazol-4-yl)pyridin-3-yl)-4-oxo-4H-chromen-8-yl)ethyl)amino)picolinic acid is prepared in accordance with the method of Example 165.

¹H NMR (400 MHz, DMSO-d₆) δ ppm 8.93 (d, J=2.00 Hz, 1H) 8.40 (s, 1H) 8.31 (d, J=6.63 Hz, 1H) 8.17-8.20 (m, 1H) 8.11 (s, 1H) 7.84 (d, J=8.38 Hz, 1H) 7.79 (s, 1H) 7.52 (d, J=2.00 Hz, 1H) 7.29 (d, J=9.01 Hz, 1H) 7.09 (d, J=9.13 Hz, 1H) 5.21 (t, J=6.69 Hz, 1H) 3.92 (s, 3H) 2.37 (s, 3H) 2.12 (s, 3H) 1.62 (d, J=6.63 Hz, 3H)

### Example 187

### (R)-2-((1-(6-methyl-2-(4-((4-methylpiperazin-1-yl)methyl)phenyl)-4-xo-4H-chromen-8-yl)ethyl)amino)benzoic acid

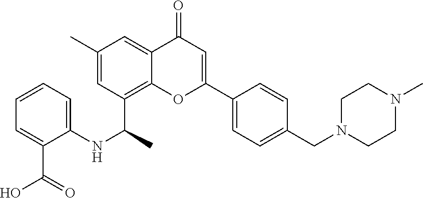 [SMILES: Cc1cc([C@@H](C)Nc2ccccc2C(=O)O)c2oc(-c3ccc(CN4CCN(C)CC4)cc3)cc(=O)c2c1]

(R)-2-((1-(6-methyl-2-(4-((4-methylpiperazin-1-yl)methyl)phenyl)-4-oxo-411-chromen-8-yl)ethyl)amino)benzoic acid is prepared in accordance with the method of Example 164.

¹H NMR (400 MHz, ME, TFIANOL-d₄) δ ppm 8.02-8.09 (m, 2H) 7.92 (br d, J=7.88 Hz, 1H) 7.85 (s, 1H) 7.69 (s, 1H) 7.58 (br d, J=7.50 Hz, 2H) 7.12 (br t, J=7.50 Hz, 1H) 6.92-6.97 (m, 1H) 6.55 (t, J=7.44 Hz, 1H) 6.44 (d, J=8.25 Hz, 1H) 5.31-5.38 (m, 1H) 3.70 (s, 2H) 2.94 (br s, 4H) 2.67 (br s, 4H) 2.60-2.63 (m, 3H) 2.41 (s, 3H) 1.74 (br d, J=4.63 Hz, 3H).

MS m/z (ESI): 512 [M+H]⁺.

### Example 188

### (R)-2-((1-(3,6-dimethyl-2-(6-((4-methylpiperazin-1-yl)methyl)pyridin-3-yl)-4-oxo-4H-chromen-8-yl)ethyl)amino)benzoic acid

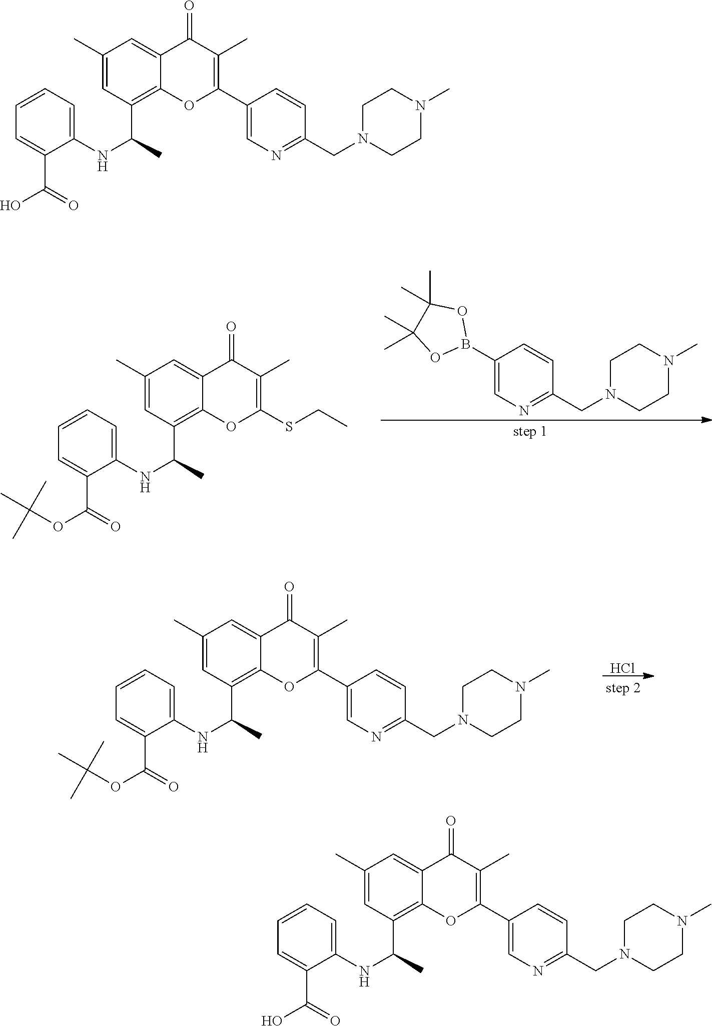

### Step 1

A mixture of tert-butyl 2-[[(1R)-1-(2-ethylsulfanyl-3,6-dimethyl-4-oxo-chromen-8-yl) ethyl]amino]benzoate (50 mg, 110.23 umol, 1 eq), 1-methyl-4-((5-(4,4,5,5-tetramethyl-1,3,2-dioxaborolan-2-yl)pyridin-2-yl)methylpiperazine (prepared in accordance with the method in patent WO202217408). (130 mg, 551.16 umol, 5 eq), cyclopentyl (diphenyl)phosphane; dichloromethane; dichloropalladium; iron (361 mg, 44.09 umol, 0.4 eq), thiophene-2-carbonyloxycopper (84 mg, 440.92 u mol, 4 eq) and Cs₂CO₃ (72 mg, 220.46 umol, 2 eq) in dioxane (1.5 mL) was degassed and purged with N₂ for 3 times, and then the mixture was stirred at 50° C. for 16 hr under N₂ atmosphere. LC MS showed one main peak with desired mass was detected. The reaction mixture was concentrated under vacuum to give the crude product. The crude product was purified by flash silica gel chromatography (ISCO®; 4 g SepaFlash® Silica Flash Column, Eluent of 0˜31% Methanol/Dichloro methane @16 mL/min). Compound tert-butyl 2-[[(1R)-1-[3,6-dimethyl-2-[6-[(4-methylpiperazin-1-yl)methyl]-3-pyridyl]-4-oxo-chromen-S-yl]ethyl]amino]benzoate (25 mg, crude) was obtained as a yellow solid.

MS m/z (ESI): 583 [M+H]⁺.

### Step 2

To a solution of tert-butyl 2-[[(1R)-1-[3,6-dimethyl-2-[6-[(4-methylpiperazin-1-yl) methyl]-3-pyridyl]-4-oxo-chromen-8-yl]ethyl]amino]benzoate (20 mg, 34.32 μmol, 1 eq) in MeCN (1 mL) was added HCl (12 M, 11.44 μL, 4 eq). The mixture was stirred at 80° C. for 2 hr. LCMS showed one main peak with desired mass was detected. The reaction mixture was concentrated under vacuum to give the crude product. The crude product was purified by prep-HPLC (column: Welch Xtimate C18 150*30 mm*5 um; mobile phase: [water (FA)-ACN]; 13%: 0%-38%, 5 min). Compound 2-[[(1R)-1-[3,6-dimethyl-2-[6-[(4-methylpiperazin-1-yl)methyl]-3-pyridyl]-4-oxo-chromen-8-yl]ethyl]amino]benzoic acid (2 ng, 3.80 μmol, 11% yield) was obtained as a yellow solid.

¹H NMR (400 MHz, DMSO-d₆) δ ppm 8.89-8.97 (m, 1H) 8.39-8.63 (m, 11H) 8.19 (dd, J=8.13, 2.13 Hz, 1H) 7.74-7.81 (n, 2H) 7.60-7.64 (m, 1H) 7.56-7.59 (m, 1H) 7.14 (s, 1H) 6.49 (t, J=7.38 Hz, 1H) 6.44 (d, J=8.50 Hz, 1H) 4.96-5.15 (m, 1H) 3.74 (s, 2H) 2.57 (br d, J=2.00 Hz, 2H) 2.51-2.51 (m, 2H) 2.49-2.49 (m, 2H) 2.45-2.48 (m, 2H) 2.38 (s, 3H) 2.20-2.26 (m, 3H) 2.08 (s, 3H) 1.61 (d, J=6.63 Hz, 3H)

MS m/z (ESI): 527 [M+H]⁺.

### Example 189

### (R)-6-chloro-3-((1-(3,6-dimethyl-2-(4-((4-methylpiperazin-1-yl)methyl)phenyl)-4-oxo-4H-chromen-8-yl)ethyl)amino)picolinic acid

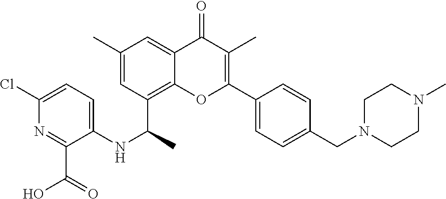 [SMILES: Cc1cc([C@@H](C)Nc2ccc(Cl)nc2C(=O)O)c2oc(-c3ccc(CN4CCN(C)CC4)cc3)c(C)c(=O)c2c1]

(R)-6-chloro-3-((1-(3,6-dimethyl-2-(4-((4-methylpiperazin-1-yl)methyl)phenyl)-4-oxo-4H-chromen-8-yl)ethyl)amino)picolinic acid is prepared in accordance with the method of Example 165.

¹H NMR (400 MHz, DMSO-d₆) δ ppm 8.89 (br d, J=6.50 Hz, 1H) 8.15 (s, 1H) 7.79 (s, 1H) 7.72 (d, J=8.00 Hz, 2H) 7.63 (d, J=1.88 Hz, 1H) 7.45 (d, J=8.13 Hz, 2H) 7.14 (d, J=8.76 Hz, 1H) 6.94 (d, J=9.01 Hz, 1H) 4.92-5.01 (m, 1H) 3.71 (s, 2H) 2.69-2.88 (m, 4H) 2.54-2.69 (m, 4H) 2.43 (s, 3H) 2.40 (s, 3H) 2.08 (s, 3H) 1.65 (d, J=6.75 Hz, 3H) MS ma/z (ESI): 561 [M+H]⁺.

### Example 190

### methyl (R)-2-((1-(3,6-dimethyl-2-(6-(1-methyl-1H-pyrazol-4-yl)pyridin-3-yl)-4-oxo-4H-chromen-8-yl)ethyl)amino)benzoate

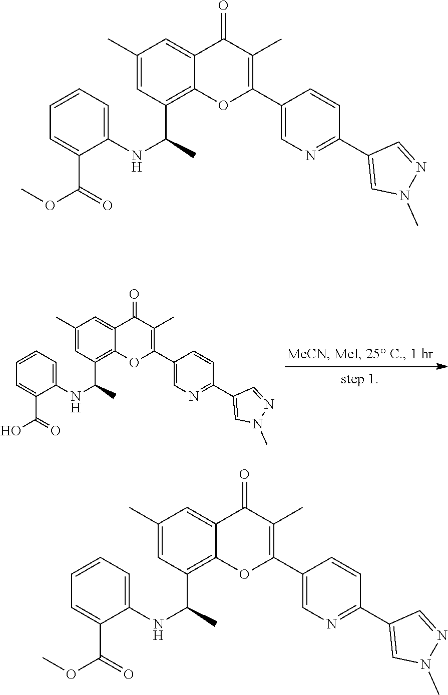

### Step 1

To a solution of 2-[[(1R)-1-[3,6-dimethyl-2-[6-(1-methylpyrazol-4-yl)-3-pyridyl]-4-oxo-chromen-8-yl]ethyl]amino]benzoic acid (20 mg, 40.44 μmol, 1 eq) and Cs₂CO₃ (20 mg, 60.66 μmol, 1.5 eq) in DMF (2 mL) was stirred at 25° C. for 15 min, was added MeI (11 mg, 80.88 μmol, 5.04 μL, 2 eq). The mixture was stirred at 25° C. for 1 hr. LCMS showed one main peak with desired mass was detected. The reaction mixture was concentrated under vacuum to give the crude product. The crude product was purified by prep-HPLC: (column: Welch Xtimate C18 150*30 mm*5 um; mobile phase: [water (FA)-A CN]; B %: 42%-82% N, 25 min). Compound methyl 2-[[(1R)-1-[3,6-dimethyl-2-[6-(1-methylpyrazol-4-yl)-3-pyridyl]-4-oxo-chromen-8-yl]ethyl] amino]benzoate; (10 mg, 18.73 μmol, 46% yield, 95% purity) was obtained as a white solid.

¹H NMR (400 MHz, DMSO-d₆) δ ppm 8.93 (d, J=1.75 Hz, 1H) 8.42 (s, 1H) 8.19-8.22 (m, 1H) 8.18 (d, J=2.25 Hz, 1H) 8.12 (s, 1H) 7.85 (d, J=8.25 Hz, 1H) 7.74-7.82 (m, 2H) 7.56 (d, J=1.88 Hz, 1H) 7.20-7.27 (m, 1H) 6.56 (br d, J=3.25 Hz, 1H) 6.54 (d, J=4.25 Hz, 1H) 5.18 (br t, J=6.63 Hz, 1H) 3.93 (s, 3H) 3.79 (s, 3H) 2.38 (s, 3H) 2.12 (s, 3H) 1.63 (d, J=6.63 Hz, 3H)

MS mm/z (ESI): 509[M+H]⁺.

### Example 191

### (R)-2-((1-(3,6-dimethyl-2-(6-(1-methyl-1H-pyrazol-4-yl)pyridin-3-yl)-4-oxo-4H-chromen-8-yl)ethyl)amino)-N-methylbenzamide

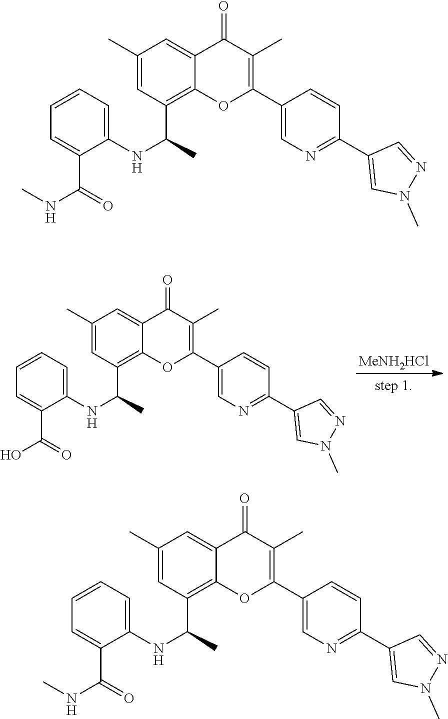 [SMILES: "MeNH2HCl" ; CNC(=O)c1ccccc1N[C@H](C)c1cc(C)cc2c(=O)c(C)c(-c3ccc(-c4cnn(C)c4)nc3)oc12 ; Cc1cc([C@@H](C)Nc2ccccc2C(=O)O)c2oc(-c3ccc(-c4cnn(C)c4)nc3)c(C)c(=O)c2c1 ; CNC(=O)c1ccccc1N[C@H](C)c1cc(C)cc2c(=O)c(C)c(-c3ccc(-c4cnn(C)c4)nc3)oc12]

### Step 1

To a solution of 2-[[(1R)-1-[3,6-dimethyl-2-[6-(1-methylpyrazol-4-yl)-3-pyridyl]-4-oxo-chromen-8-yl]ethyl]amino]benzoic acid (20 mg, 40.44 μmol, 1 eq) in DMF (1 mL) was added mnethanamine; hydrochloride (3 mg, 48 μmol, 1.2 eq), DIEA (26 mg, 202 μmol, 35 μL, 5 eq) and HATU (23 mg, 60 μmol, 1.5 eq). The mixture was stirred at 25° C. for 0.5 hr. The reaction mixture was filtered to remove the insoluble to give the residue. The residue was purified by prep-HPLC (column: Welch Xtimate CIS 150*30 mm*5 um; mobile phase: [water (FA)-ACN]; B %: 24%-64%, 25 min). Compound 2-[[(1R)-1-[3,6-dimethyl-2-[6-(1-methylpyrazol-4-yl)-3-pyridyl]-4-oxo-chromen-8-yl]ethyl]amino]-N-methyl-benzamide (13 mg. 24.05 μmol, 59% yield, 94% purity) was obtained as a white solid.

¹H NMR (400 MHz, DMSO-d₆) δ ppm 8.95 (d, J=2.13 Hz, 1) 8.52 (d, J=6.63 Hz, 1H) 8.42 (s, 1H) 8.35 (br d, J=4.63 Hz, 1H) 8.21 (dd, J=8.26, 2.25 Hz, 1H) 8.12 (s, 1H) 7.86 (d, J=8.38 Hz, 1H) 7.77 (s, 1H) 7.50-7.55 (m, 2H) 7.10 (t, J=7.57 Hz, 1H) 6.53 (t, J=7.57 Hz, 1H) 6.43 (d, J=8.38 Hz, 1H) 5.10 (br t, J=6.63 Hz, 1H) 3.93 (s, 3H) 2.76 (d, J=4.50 Hz, 3H) 2.36 (s, 3H) 2.14 (s, 3H) 1.57 (d, J=6.63 Hz, 3H)

MS m/z (ESI): 508 [M+H]⁺.

### Example 192

### (R)-2-((1-(2-(6-(2-(dimethylamino)ethoxy)pyridin-3-yl)-3,6-dimethyl-4-oxo-4H-chromen-8-yl)ethyl)amino)benzoic acid

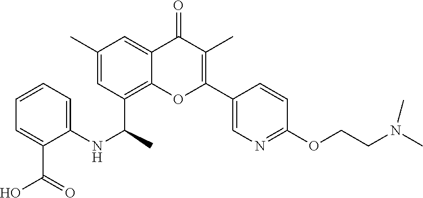 [SMILES: Cc1cc([C@@H](C)Nc2ccccc2C(=O)O)c2oc(-c3ccc(OCCN(C)C)nc3)c(C)c(=O)c2c1]

(R)-2-((1-(2-(6-(2-(dimethylamino)ethoxy)pyridin-3-yl)-3,6-dimethyl-4-oxo-4H-chromen-8-yl)ethyl)amino)benzoic acid is prepared in accordance with the method of Example 165.

¹H NMR (400 MHz, DMSO-d) δ ppm 8.63 (d, J=2.00 Hz, 1H) 8.38-8.48 (m, 1H) 8.12 (dd, J=8.69, 2.31 Hz, 1H) 7.71-7.85 (m, 2H) 7.52 (d, J=1.63 Hz, 1H) 7.18 (br t, J=7.94 Hz, 1H) 7.01 (d, J=8.63 Hz, 1H) 6.53 (t, J=7.57 Hz, 1H) 6.44 (d, J=8.38 Hz, 1H) 5.12 (brt, J=6.38 Hz, 1H) 4.46 (t, J=5.75 Hz, 2H) 2.71 (br t, J=5.32 Hz, 2H) 2.36 (s, 3H) 2.26 (s, 6H) 2.09 (s, 3H) 1.58 (d, J=6.63 Hz, 3H)

MS m/z (ESI): 502[M+H]⁺.

### Biological Evaluation

Exemplary compounds of Formula (VI), (VII), (VII-1) or (VIII) are tested to assess compound inhibition of PI3Kα.

### 1. PI3Kα Kinase (PIK3CA) Activity IC₅₀ Assay

Recombinant, catalytically active human full length PIK3KA wild-type and 11047R mutant, E545K mutant are purchased as 1:1 complex of N-terminal 6× his tagged p110α (catalytic) and untagged p85α (regulatory subunit) from Merck Millipore (cat. no. 14-602, 14-792 and 14-783, respectively). The enzyme stocks are diluted to 5× stocks in buffer (20 mM HEPES pH7.4, 100 mM NaCl, 0.5 mM EGTA, 0.01% triton-X-100) just before use. PI 3-Kinase 3-step HTRF™ Assay kits are purchased from Merck Millipore (cat. no 33-040) with P1P2 substrate, 4× reaction buffer, DMA, DMB, DMC reaction buffer, stop reaction buffer and BPIP3.

For each PI3-Kinase Assay:
a. Outline the wells required of a black 384 well plate.
b. Add 0.5 μl of 100% DMSO or test compounds with series dilution to each well.
c. Add 14.5 μl PI3-Kinase (Wild-type (WT) and H1047R mutant, E545K mutant protein) per well.
d. For a negative control add 14.5 μl of 1× Reaction Buffer/B-PIP3/PIP2 working solution per well instead of the kinase.
e. Add 5 μl of ATP Working Solution to all wells.
f. Centrifuge the plate in a bench centrifuge for 1 minute.
g. Incubate the assay plate at room temperature for 30 minutes.
h. Combine equal volumes of Stop Solution with Detection Mix, mix and keep in the dark at Room Temperature.
i. Add 10 μl of Stop Solution/Detection Mix to all wells.
j. Centrifuge the plate in a bench centrifuge for 1 minute.
k. Incubate at room temperature for a minimum of 2 hours in the dark. It is recommended that the plate is sealed to minimize reduction in reaction volume.
l. Measure HTRF ratio by ThermoFisher Varioskan LUX multifunctional plate reader.

### Calculation:

HTRF Ratio is calculated as follows:

$$HTRF Ratio = ( \frac{Emission at 665 nm}{Emission at 620 nm} ) \times 10000$$

Test compounds are tested in the above assay, and the IC₅₀ values of test compounds fall between 0.01 to 2000 nM.

### 2. Cell Proliferation IC₅₀ Assay

PI3Kα H1047R mutation cell line T-47D and MDA-MB-453, E545K mutation cell line MDA-MB-361 and PI3Kα wide type cell line SK-BR-3 were purchased from Cobioer Biotech (cat. no CBP60397, CBP60386, CBP60383 and CBP60413 respectively). The CellTiter-Glo Direct Cell Proliferation Assay Kit was purchased from Prornega (cat. No. G7573)

For cell proliferation assay in each cell line:
a. Prepare complete medium (R PMI 1640, DMEM or McCoys): Add FBS and appropriate additives according to the information sheet provided by the vendor. Mix gently.
b. Prepare single cell suspension and count the cell numbers and adjust cells to appropriate density.
c. Add 80 μL of cell suspension to 96-well opaque-walled clear bottom plates according to the planned plate layout and place the plates in the CO2 incubator overnight.
d. Prepare intermediate plate: 10 μl of diluted test compounds or staurosporine solution (200×) is added to a 96 deep well plate. Then 390 μl of culture medium without FBS is added to the respective wells.
e. Transfer 20 μL of the solutions from the above intermediate plate to the wells of the plate prepared containing 80 μL of the culture medium and cells. The total dilution is 200 folds.
f. The cells were then incubated at 5% CO₂, 37° C. for 72 hours.
g. Equilibrate the plate and its contents to room temperature for approximately 30 minutes.
h. Add 100 μL of CellTiter-Glo reagent to the assay plate
i. Mix contents for 10 minutes on an orbital shaker to induce cell lysis.
j. Allow the plate to incubate at room temperature for 10 minutes to stabilize luminescent signal,
k. Paste the clear bottom with white back seal and record luminescence with Varioskan LUX plate reader.

### Calculation:

Inhibition % and IC₅₀ value were calculated by the formula described below:

$$Inhibition \% = [ 1 - ( {RTU}_{compound} - {RTU}_{blank} ) / ( {RTU}_{DMSO} - {RTU}_{blank} ) ] \times 100 \%$$

Test compounds were tested in the above assays, and the IC₅₀ values of test compounds were shown in Table 2.

TABLE 2

| | IC₅₀ (μM) | IC₅₀ (μM) | IC₅₀ (μM) |
|---|---|---|---|
| Compounds | SK-BR-3 | MDA-MB-453 | T47D |
| 1 | >10 | 0.70 | 0.33 |
| 2 | >10 | 0.39 | 0.16 |
| 3 | >10 | 0.56 | 0.75 |
| 4 | >10 | 0.75 | 0.56 |
| 5 | >10 | / | 1.54 |
| 6 | >10 | 0.29 | 0.23 |
| 7 | >10 | 0.25 | / |
| 10 | >10 | 2.44 | 0.86 |
| 11 | >10 | 2.49 | 1.98 |
| 13 | >10 | 2.29 | 1.33 |
| 14 | >10 | 1.27 | 1 |
| 15 | >10 | >10 | >10 |
| 16 | >10 | / | 2.56 |
| 17 | >10 | / | 2.07 |
| 18 | >10 | 0.77 | 0.31 |
| 19 | >10 | 1.39 | 0.35 |
| 20 | >10 | 0.18 | 0.12 |
| 20A | >10 | >10 | >10 |
| 20B | >10 | 0.07 | 0.048 |
| 21 | >10 | >10 | / |
| 22 | 5.07 | 0.63 | 0.72 |
| 23 | >10 | 6.3 | 3.2 |
| 24 | >10 | 4.07 | / |
| 25 | >10 | 0.32 | 0.27 |
| 26 | >10 | 0.83 | 0.66 |
| 27 | >10 | 0.74 | / |
| 28 | >10 | 3.61 | 1.77 |
| 29 | >10 | >10 | 4.13 |
| 30 | >10 | 4 | 1.87 |
| 31 | >10 | / | 0.9 |
| 32 | >10 | 1.38 | 0.75 |
| 33 | >10 | 0.42 | 0.32 |
| 34 | >10 | 7.82 | 4.63 |
| 35 | >10 | 0.57 | 0.5 |
| 36 | >10 | / | 0.2 |
| 37 | >10 | 0.8 | 0.42 |
| 38 | >10 | 0.15 | 0.08 |
| 38A | >10 | >10 | >10 |
| 38B | 8.5 | 0.05 | 0.04 |
| 39 | >10 | 2.49 | 1.98 |
| 40 | >10 | / | 2.51 |
| 41 | >10 | / | 1.43 |
| 42 | >10 | 0.2 | 0.21 |
| 43 | >10 | / | 0.28 |
| 44 | >10 | 0.39 | / |
| 45 | >10 | 0.97 | / |
| 46 | >10 | 0.42 | / |
| 47 | >10 | 0.24 | / |
| 48 | >10 | 0.76 | / |
| 49A | >10 | 0.09 | 0.07 |
| 49B | >10 | / | 7.24 |
| 50 | >10 | 6.39 | / |
| 51 | >10 | 2.48 | / |
| 52 | >10 | 0.78 | / |
| 53A | >10 | 0.12 | 0.09 |
| 53B | >10 | 2.34 | / |
| 56 | >10 | 0.55 | / |
| 57 | >10 | 0.62 | / |
| 58 | >10 | 0.27 | / |
| 60 | >10 | 6.0 | / |
| 61 | >10 | 8.48 | / |
| 62 | >10 | 0.47 | / |
| 64 | >10 | 1.9 | / |
| 65 | >10 | 0.21 | / |
| 66 | >10 | 0.20 | / |
| 67 | >10 | 0.20 | / |
| 69 | >10 | 1.06 | / |
| 72 | >10 | / | 5.08 |
| 73 | >10 | / | 2.01 |
| 81 | >10 | / | 0.15 |
| 82 | >10 | / | >10 |
| 83 | >10 | / | 0.38 |
| 84 | >10 | 0.26 | / |
| 85 | >10 | / | 0.13 |
| 90 | >10 | 0.09 | / |
| 93 | >10 | / | 0.31 |
| 94 | >10 | / | 1.02 |
| 95A | >10 | / | 0.09 |
| 95B | >10 | / | 0.99 |
| 98A | >10 | 0.09 | 0.038 |
| 98B | >10 | >10 | >10 |
| 101 | >10 | / | 0.29 |
| 108 | >10 | / | 0.84 |
| 109 | >10 | 0.16 | 0.22 |
| 110 | >10 | / | 0.087 |
| 111 | >10 | 0.87 | / |
| 112 | >10 | / | >10 |
| 113 | >10 | / | 0.67 |
| 114 | >10 | / | 0.46 |
| 115 | 3.43 | / | 0.074 |
| 116 | >10 | / | 0.97 |
| 117 | >10 | / | 0.28 |
| 118 | 7.52 | 0.14 | / |
| 119A | >10 | >10 | / |
| 119B | >10 | 0.75 | / |
| 120 | >10 | 1.18 | / |
| 121 | >10 | / | 0.76 |
| 122 | >10 | 0.56 | / |
| 123A | 5.6 | 0.15 | 0.076 |
| 123B | >10 | >10 | >10 |
| 124 | >10 | 0.5 | / |
| 125 | >10 | 0.4 | / |
| 126 | >10 | 2.46 | / |
| 127 | >10 | >10 | / |
| 128 | >10 | 0.16 | / |
| 129 | >10 | 0.26 | / |
| 130 | >10 | >10 | / |
| 131 | >10 | 0.059 | 0.055 |
| 132 | >10 | 0.27 | / |
| 133 | >10 | 1.18 | / |
| 134 | >10 | 0.56 | / |
| 135 | >10 | / | 0.18 |
| 136 | >10 | / | >10 |
| 137 | >10 | / | 0.19 |
| 138 | >10 | / | 0.083 |
| 139 | >10 | / | 1.04 |
| 140 | >10 | 0.029 | 0.041 |
| 141 | >10 | / | 0.044 |
| 142 | >10 | / | 0.043 |
| 143 | >10 | / | 0.25 |
| 144 | >10 | / | 0.14 |
| 145 | >10 | / | 1.6 |
| 146 | >10 | / | 0.18 |
| 147 | >10 | / | 0.055 |
| 148 | >10 | / | 0.066 |
| 149A | >10 | / | 0.11 |
| 149B | >10 | / | >10 |
| 150 | >10 | 0.13 | 0.064 |
| 151 | >10 | 0.089 | 0.058 |
| 152 | >10 | / | 6.9 |
| 153 | >10 | / | 0.29 |
| 154 | >10 | 0.086 | 0.074 |
| 155A | >10 | / | 0.12 |
| 155B | >10 | / | 5.06 |
| 156 | >10 | / | 0.13 |
| 157 | >10 | / | 3.43 |
| 158 | >10 | / | 5.5 |
| 159 | >10 | / | 0.41 |
| 160 | >10 | / | 0.065 |
| 161 | 6.5 | / | 0.067 |
| 162 | >10 | / | 0.23 |
| 163 | >10 | / | 0.12 |
| 164 | >10 | / | 0.38 |
| 165 | 4.39 | / | 0.042 |
| 166 | >10 | / | 0.23 |
| 167 | 8.34 | / | 0.092 |
| 168 | >10 | / | 0.092 |
| 169 | >10 | / | 0.11 |
| 170 | >10 | / | 0.096 |
| 171 | >10 | / | 0.072 |
| 172A | >10 | / | 0.065 |
| 172B | >10 | / | 0.063 |
| 176 | >10 | / | >10 |
| 177 | >10 | / | 0.31 |
| 186 | 8.68 | / | 0.16 |
| 188 | >10 | / | 0.13 |
| 189 | >10 | / | 0.11 |
| 190 | 6.05 | / | 0.27 |
| 191 | 2.02 | / | 0.27 |
| 192 | >10 | / | 0.10 |

### 3. Selectivity Study Against 783-O Cell (PI3Kβ)

To evaluate the potency and efficacy of selected compound against PI3K-beta activity, 786-O cells were seed at a density of 30,000 cells/well in a 96-well culture plate and allowed to adhere 18 hours at 37° C./5% CO₂. The following day, the complete medium was replaced with serum-free medium, 3-fold serial diluted compound solution in serum-free medium was added to each well according to the plate map. After 1 hour incubation, removed cell supernatant carefully and added supplemented lysis buffer (1×) for 45 min. The lysate was transferred to a 384-well small volume white plate, the CisBio Phospho-AKT (Ser473) HTRF assay was performed as the manufacturer's directions (Cisbio, #64AKSPEG).

TABLE 3
Selective for PI3Kβ with 783-O cell in p-AKT HTRF assay.

| Example | PI3Kβ IC50 (nM) |
|---|---|
| 38B | 545 |
| 95 | 1218 |
| 98 | 1146 |
| 110 | >10000 |
| 123A | 6455 |
| 140 | >10000 |
| 147 | >10000 |
| 148 | >10000 |
| 150 | >10000 |

### 4. pAKT (S473) ELISA Assay in PI3Kα H1047R Mutant Cell Lines and WT Cell Lines

To evaluate the potency and efficacy of selected compound against PI3K-alpha activity, T47D and SK-BR-3 cells were seed at a density of 120,000 cells/well in a 6-well culture plate and allowed to adhere overnight. Cells were serum starved for 4 hours prior to compound treatment; compound solution was added to each well (3-fold serial dilution starting at desired concentration) for two hours in serum-free medium, followed by stimulation with IGF-1 for 60 minutes at a final concentration of 50 ng/ml as noted. After compound treatment and stimulation, discarded the supernatant and cells were lysed by cell lysis buffer (Cell Signaling Technology, #9803S). The lysate was transferred to the ELISA plate, the Phospho-AKT (Ser473) ELISA was performed as the manufacturer's directions (Cell Signaling Technology, #7160CA).

TABLE 4
pAKT(S473) Elisa assay results.

| Example | IC₅₀(nM) H1047R mutant | IC₅₀(nM) WT |
|---|---|---|
| 38B | 25.0 | 1291 |
| 95A | 12.0 | 1258 |
| 98B | 19.5 | 1337 |
| 123A | 37.0 | 1096 |
| 140 | 42.3 | 2391 |
| 147 | 46.5 | 6129 |
| 148 | 48.5 | 4337 |
| 150 | 25.0 | 2374 |

### 5. In Vivo Pharmacokinetics Study

### Pharmacokinetics in Mice

Dosing solutions were prepared at 0.5 mg/mL in 10% DMSO/10% HP—3-CD/80% water. A dosing solution was administered to Male CD1 mice (4-6 weeks, 20-30 g, 3 mice each group) via intravenous (TV) bolus at 1 mg/kg and by oral gavage (PO) at 5 mg/kg. The dose formulations were kept at room temperature and administrated within 30 mins for IV/PO. Blood samples (0.3 mL each time point) were collected into tubes containing ethylenediaminetetraacetic acid (K₂EDTA) as the anticoagulant at 0, 0033, 0.083, 0.25, 05, 1, 2, 4, 8 and 24 hours post dose for IV administrated and 0, 0.083, 0.25, 0.5, 1, 2, 4, 8 and 24 hours post dose for PO administration. The blood sample were then centrifuged for 5 minutes in centrifuge refrigerated at 4° C. The samples were stored in a freezer at −75±15° C. prior to analysis. Concentrations of Compound in the plasma samples were analyzed using a LC-MS/MS method. WinNonlin (Phoenix™, version 8.3) or other similar software were used for pharmacokinetic calculations. The following pharmacokinetic parameters were calculated, whenever possible from the plasma concentration versus time data: MRT, Cmax, Tmax, AUClast, AUCinf, F %, Number of Points for Regression. The PIK results are listed in Table 5.

TABLE 5
Mouse PK parameters for exemplary Example in mice.

| | Cl (mL/min/kg) | Cmax (ng/mL) | MRT (h) | F (%) |
|---|---|---|---|---|
| Example 38B | 34 | 87 | 6.7 | 45.5 |
| Example 95A | / | 102 | 7.64 | / |
| Example 147 | 10.9 | 628 | 6.6 | 46.5 |
| Example 148 | / | 566 | 7.04 | / |
| Example 150 | 3.75 | 895 | 7.92 | 58.2 |
| Example 169 | / | 653 | 13.6 | / |

¹IV bolus at 1 mg/kg and PO at 5 mg/kg in CD1 mice

1. A compound of formula (VII), a stereoisomer thereof, or a pharmaceutically acceptable salt, solvates and prodrugs thereof: 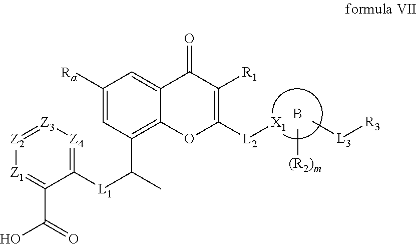
Wherein,
Each Z₁, Z₂, Z₃ and Z₄ is independently selected from CH, N or CR_{c};
R_{c} is each independently selected from hydrogen, deuterium, C₁-C₆ alkyl, deuterated C₁-C₆ alkyl, C₁-C₆ haloalkyl, C₁-C₆ alkoxy, C₁-C₆ haloalkoxy, —S(O)₂-alkyl, halogen, amino, nitro, hydroxy, cyano;
L₁ is independently selected from —N(H)—, —N(C₁-C₆ alkyl)-, —O—, —S— and —CH—;
Rₐ is independently selected from hydrogen, deuterium, C₁-C₆ alkyl, deu terated C₁-C₆ alkyl, C₁-C₆ haloalkyl, C₁-C₆ hydroxyalkyl, C₁-C₆ alkoxy, C₁-C₆ haloalkoxy, halogen, amino, nitro, hydroxy, cyano, C₃-C₉ cycloalkyl, 4 to 10 membered heterocyclyl, C₆-C₁₀ aryl and 5 to 10 membered heteroaryl;
R₁ is independently selected from hydrogen, deuterium, halogen, hydroxy, cyano, C₁-C₆ alkyl, deuterated C₁-C₆ alkyl, C₁-C₆ haloalkyl, C₁-C₆ alkoxy C₁-C₆ haloalkoxy, C₃-C₅ cycloalkyl and 5 or 6 membered heteroaryl;
L₂ is a bond, 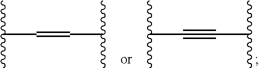
X₁ is a ring atom of ring B and ring B is connected to L₂ through X₁ atom, wherein X₁ is a carbon atom;
Ring B is independently selected from C₃-C₈ cycloalkyl, 4 to 10 membered heterocyclyl, C₆-C₁₀ aryl and 5 to 10 membered heteroaryl;
R₂ is identical or different and each is independently selected from hydrogen, deuterium, oxo, halogen, hydroxy, amino, nitro, cyano, ester group, C₁-C₆ alkyl, deuterated C₁-C₆ alkyl, C₁-C₆ hydroxyalkyl, C₁-C₆ haloalkyl, C₁-C₆ alkoxy, C₃-C₈ cycloalkyl, 4 to 10 membered heterocyclyl, C₆-C₁₀ aryl and 5 to 10 membered heteroaryl, wherein C₃-C₈ cycloalkyl, 4 to 10 membered heterocyclyl, C₆-C₁₀ aryl and 5 to 10 membered heteroaryl are each optionally further substituted by one or more substituents selected from deuterium, halogen, hydroxy, amino, nitro, cyano, ester group, carboxyl, C₁-C₆ alkyl, deuterated C₁-C₆ alkyl, C₁-C₆ hydroxyalkyl, C₁-C₆ haloalkyl and C₁-C₆ alkoxy.
L₃ is independently selected from bond, —O—, —(CH₂)₁₋₄—, —C(O)—, —C(O)N(H)—, —N(H)C(O)—, —C(O)N(C₁-C₆ alkyl)- and —N(C₁-C₆ alkyl)C(O)—, —S(O)₂—, —(CH₂)₁₋₄—C(O)—, —O—(CH₂)₁₋₄—, —CH(C₁-C₆ alkyl)-, C₃-C₈ cycloalkyl, 4 to 10 membered heterocyclyl, C₁-C₁₀ aryl and 5 to 10 membered heteroaryl.
R₃ is independently selected from hydrogen, deuterium, halogen, hydroxy, amino, C₁-C₆ alkyl, deuterated C₁-C₆ alkyl, C₁-C₆ hydroxyalkyl, C₁-C₆ haloalkyl, C₁-C₆ alkoxy, —NH(C₁-C₆ alkyl), —N(C₁-C₆ alkyl)₂, C₃-C₈ cycloalkyl, 4 to 10 membered heterocyclyl, C₆-C₁₀ aryl and 5 to 10 membered heteroaryl, wherein C₃-C₈ cycloalkyl, 4 to 10 membered heterocyclyl, C₆-C₁₀ aryl and 5 to 10 membered heteroaryl are each optionally further substituted by one or more hydrogen, deuterium, oxo, halogen, hydroxy, amino, nitro, cyano, ester group, carboxyl, C₁-C₆ alkyl, C₁-C₆ hydroxyalkyl, C₁-C₆ haloalkyl and C₁-C₆ alkoxy, —S(O)₂R₃₁ and —C(O)R₃₁.
R₃₁ is independently selected from hydrogen, halogen, hydroxy, amino, C₁-C₆ alkyl, deuterated C₁-C₆ alkyl, C₁-C₆ hydroxyalkyl, C₁-C₆ haloalkyl, C₁-C₆ alkoxy, —NH(C₁-C₆ alkyl) and —N(C₁-C₆ alkyl)₂.
m is an integer of 0, 1, 2, 3, 4 or 5.

2. The compound according to claim 1, a stereoisomer thereof, or a pharmaceutically acceptable salt, solvates and prodrugs thereof, wherein each 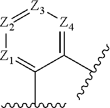
is independently selected from

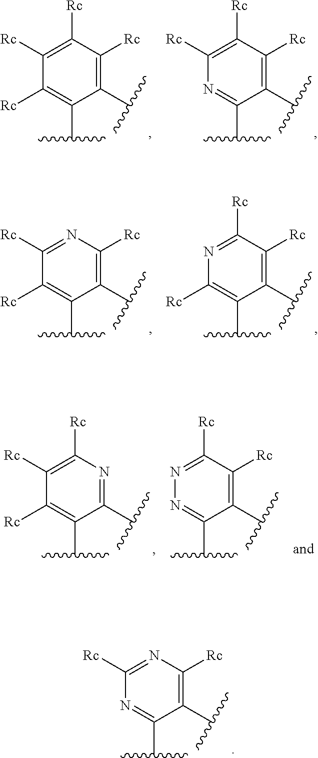

3. The compound according to claim 1, a stereoisomer thereof, or a pharmaceutically acceptable salt, solvates and prodrugs thereof, wherein each R, is independently selected from hydrogen, halogen, hydroxy, cyano, C₁-C₄ alkyl, C₁-C₄ haloalkyl and C₁-C₄ alkoxy.

4. The compound according to claim 1, a stereoisomer thereof, or a pharmaceutically acceptable salt, solvates and prodrugs thereof, wherein each Rₐ is independently selected from hydrogen, halogen, hydroxy, cyano, C₁-C₄ alkyl, C₁-C₄ haloalkyl, C₁-C₄ hydroxyalkyl, C₁-C₄ alkoxy and C₃-C₆ cycloalkyl.

5. The compound according to claim 1, a stereoisomer thereof, or a pharmaceutically acceptable salt, solvates and prodrugs thereof, wherein each R₁ is independently selected from hydrogen, halogen, hydroxy, cyano, C₁-C₄ alkyl, deuterated C₁-C₄ alkyl, C₁-C₄ haloalkyl and C₁-C₄ alkoxy and C₃-C₆ cycloalkyl.

6. The compound according to claim 1, a stereoisomer thereof, or a pharmaceutically acceptable salt, solvates and prodrugs thereof, wherein L₂ is bond.

7. The compound according to claim 1, a stereoisomer thereof, or a pharmaceutically acceptable salt, solvates and prodrugs thereof, wherein each ring B is independently selected from

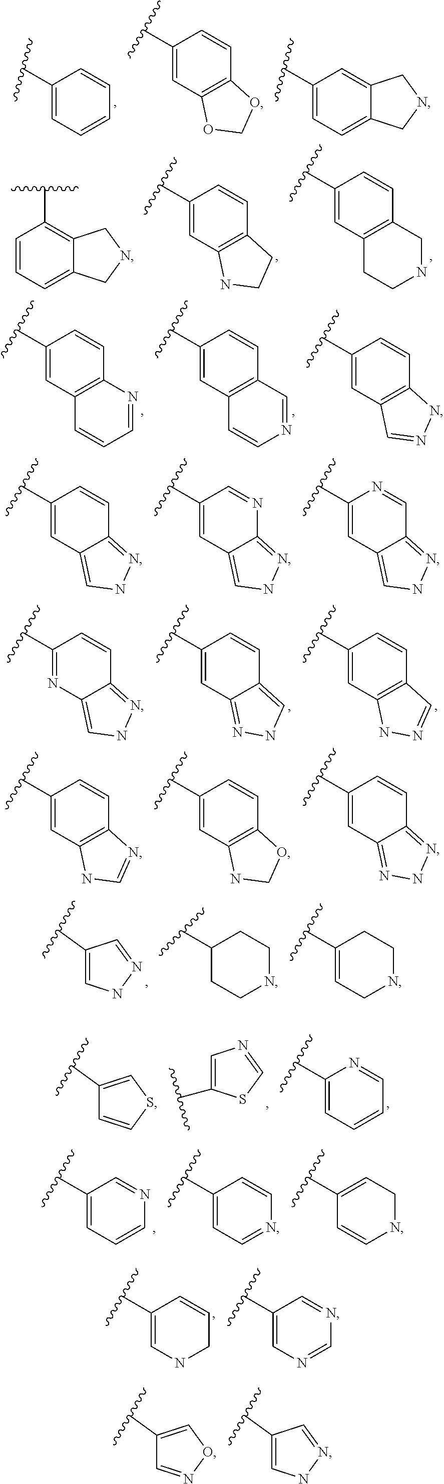

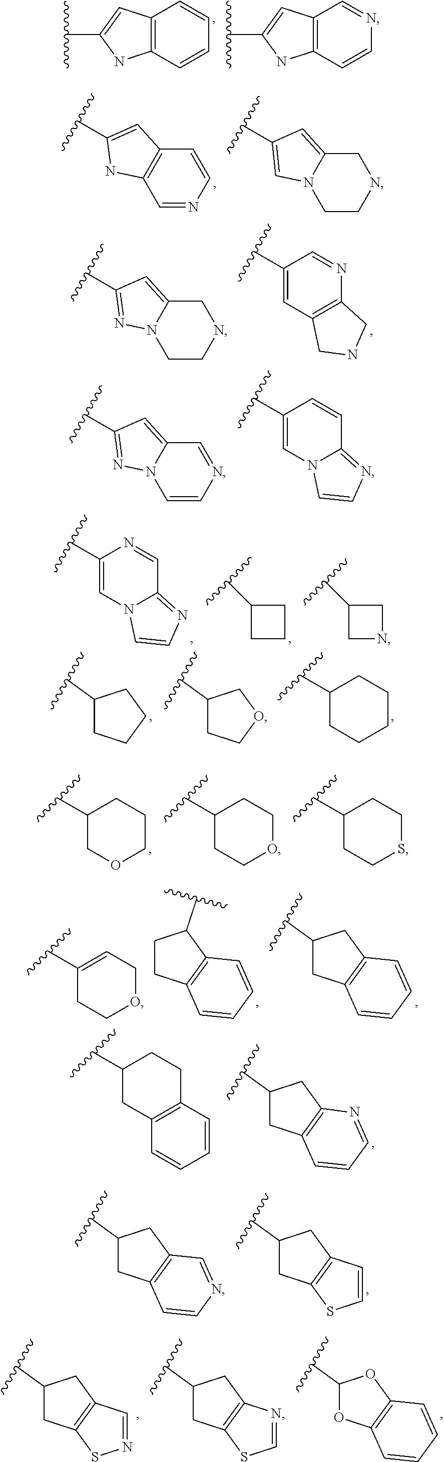

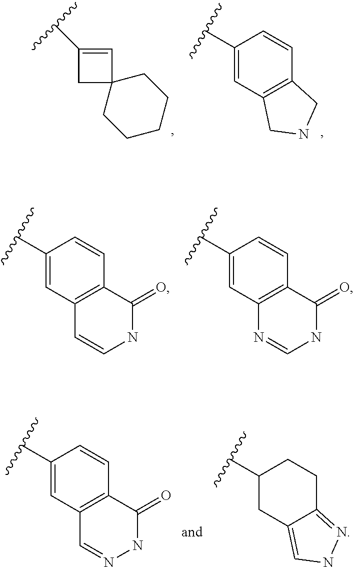

8. The compound according to claim 1, a stereoisomer thereof, or a pharmaceutically acceptable salt, solvates and prodrugs thereof, wherein each R₂ is independently selected from hydrogen, halogen, oxo, cyano, C₁-C₄ alkyl, C₁-C₄ haloalkyl, C₁-C₁ hydroxyalkyl and C₃-C₆ cycloalkyl.

9. The compound according to claim 1, a stereoisomer thereof or a pharmaceutically acceptable salt, solvates and prodrugs thereof, wherein each L₃ is independently selected from bond, —O—, —CH₂—, —(CH₂)₂—, —C(O)—, —C(O)N(H)—, —C(O)N(C₁-C₄ alkyl)₂, —S(O)₂—, —CH₂—C(O)—, —CH(C₁-C₄ alkyl)-, —O—(CH₂)₁₋₄

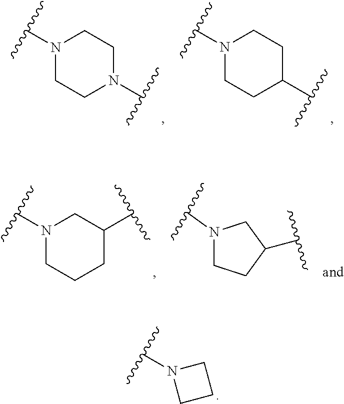 [SMILES: *N1CCN(*)CC1 ; *C1CCN(*)CC1 ; *C1CCCN(*)C1 ; *C1CCN(*)C1 ; *N1CCC1]

10. The compound according to claim 1, a stereoisomer thereof, or a pharmaceutically acceptable salt, solvates and prodrugs thereof, wherein each R₃ is independently selected from hydrogen, —N(C₁-C₆ alkyl)₂, 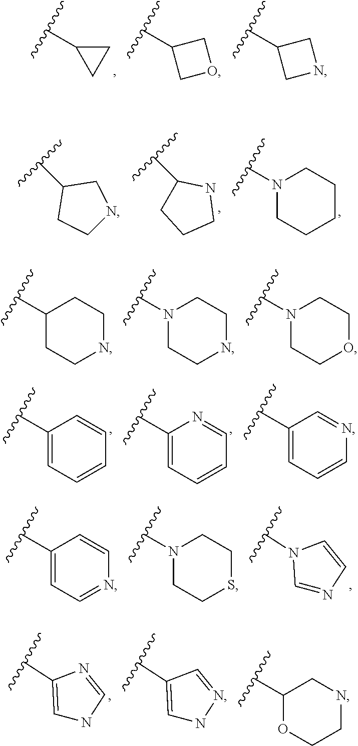
wherein each R₃ is optionally further substituted by one or more hydrogen, deuterium, oxo, halogen, hydroxy, amino, nitro, cyano, ester group, carboxyl, C₁-C₆ alkyl, C₁-C₆ hydroxyalkyl, C₁-C₆ haloalkyl and C₁-C₆ alkoxy, —S(O)₂R₃ and —C(O)R₃₁.

11. The compound according to claim 1, a stereoisomer thereof, or a pharmaceutically acceptable salt, solvates and prodrugs thereof, wherein the compound of formula (VII) is a compound of formula (VII-1): 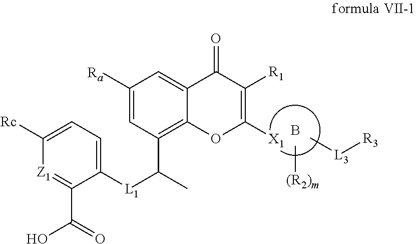
Wherein: 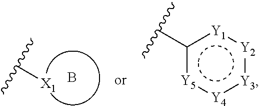
each Y₁, Y₂, Y₃, Y₄, and Y₅ is independently selected from C, CR_{y} and N;
R_{y} is hydrogen or two R_{y} and the atoms to which they are attached can form a 5 to 6 membered heterocycle or heteroaryl;
L₁, L₃, Rₐ, R_{c}, R₁, R₂, R₃, Z₁ and m are defined as in claim 1.

12. The compound according to claim 11, a stereoisomer thereof or a pharmaceutically acceptable salt, solvates and prodrugs thereof, wherein 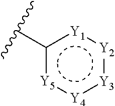
is selected from

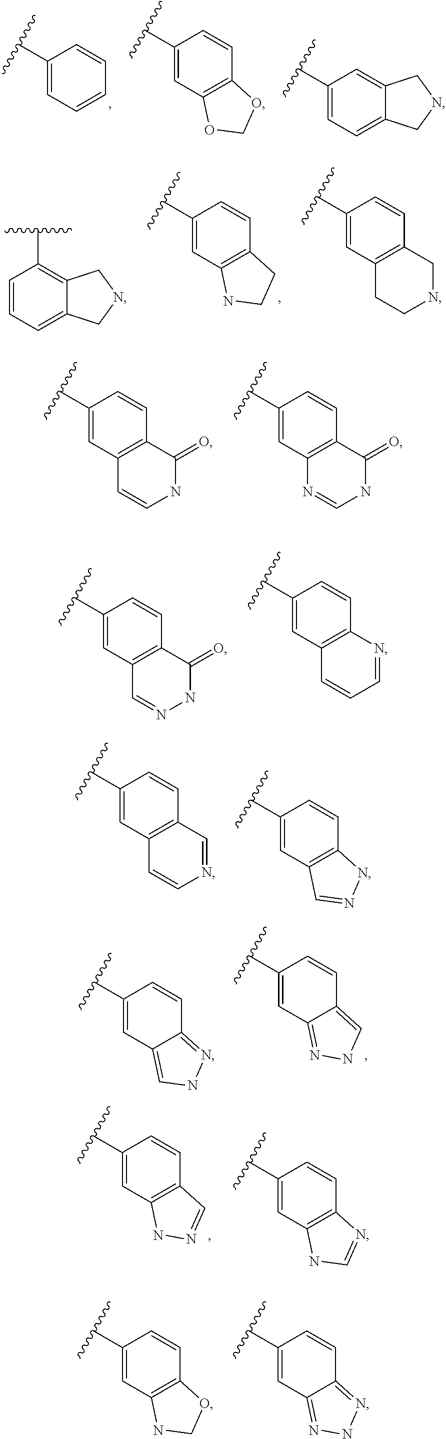

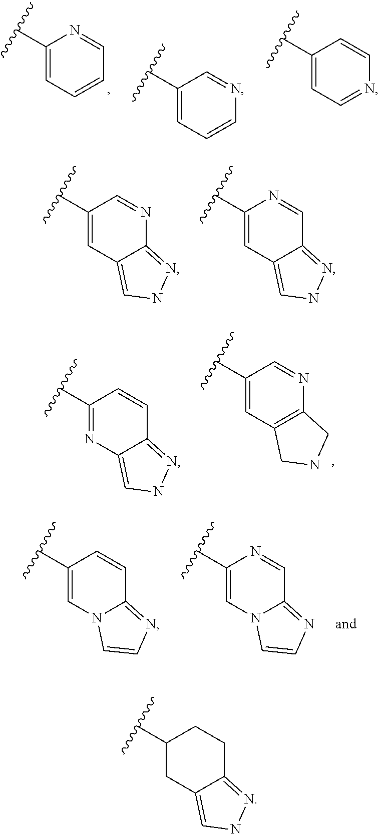

13. The compound according to claim 1, a stereoisomer thereof, or a pharmaceutically acceptable salt, solvates and prodrugs thereof, selected from

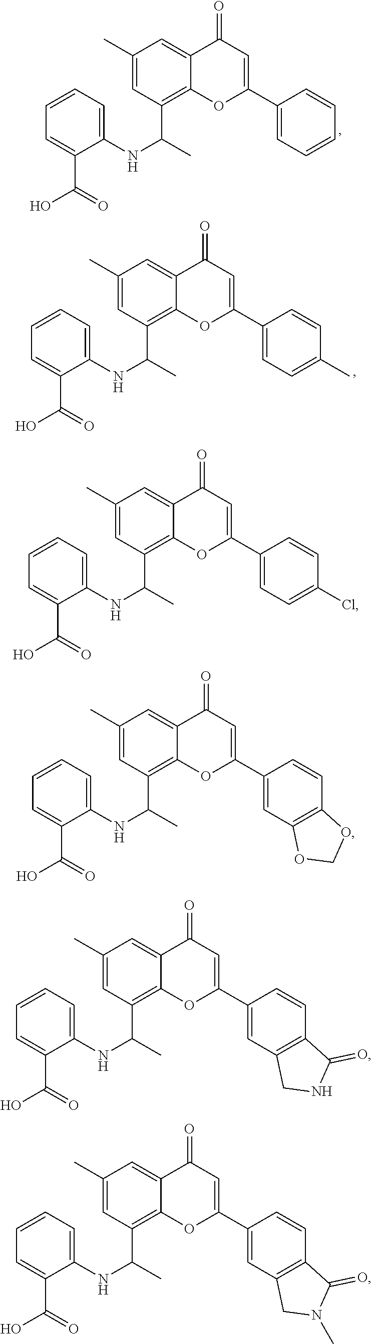 [SMILES: Cc1cc(C(C)Nc2ccccc2C(=O)O)c2oc(-c3ccccc3)cc(=O)c2c1 ; Cc1ccc(-c2cc(=O)c3cc(C)cc(C(C)Nc4ccccc4C(=O)O)c3o2)cc1 ; Cc1cc(C(C)Nc2ccccc2C(=O)O)c2oc(-c3ccc(Cl)cc3)cc(=O)c2c1 ; Cc1cc(C(C)Nc2ccccc2C(=O)O)c2oc(-c3ccc4c(c3)OCO4)cc(=O)c2c1 ; Cc1cc(C(C)Nc2ccccc2C(=O)O)c2oc(-c3ccc4c(c3)CNC4=O)cc(=O)c2c1 ; Cc1cc(C(C)Nc2ccccc2C(=O)O)c2oc(-c3ccc4c(c3)CN(C)C4=O)cc(=O)c2c1]

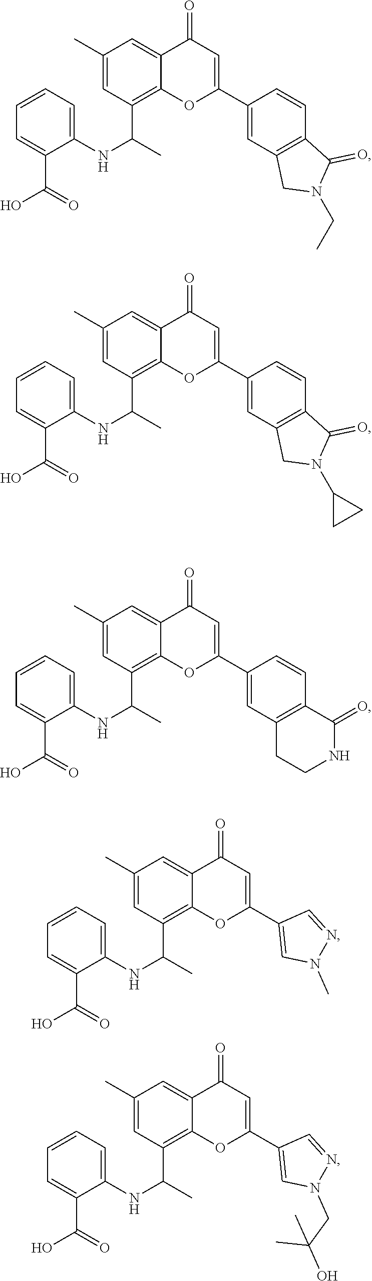 [SMILES: CCN1Cc2cc(-c3cc(=O)c4cc(C)cc(C(C)Nc5ccccc5C(=O)O)c4o3)ccc2C1=O ; Cc1cc(C(C)Nc2ccccc2C(=O)O)c2oc(-c3ccc4c(c3)CN(C3CC3)C4=O)cc(=O)c2c1 ; Cc1cc(C(C)Nc2ccccc2C(=O)O)c2oc(-c3ccc4c(c3)CCNC4=O)cc(=O)c2c1 ; Cc1cc(C(C)Nc2ccccc2C(=O)O)c2oc(-c3cnn(C)c3)cc(=O)c2c1 ; Cc1cc(C(C)Nc2ccccc2C(=O)O)c2oc(-c3cnn(CC(C)(C)O)c3)cc(=O)c2c1]

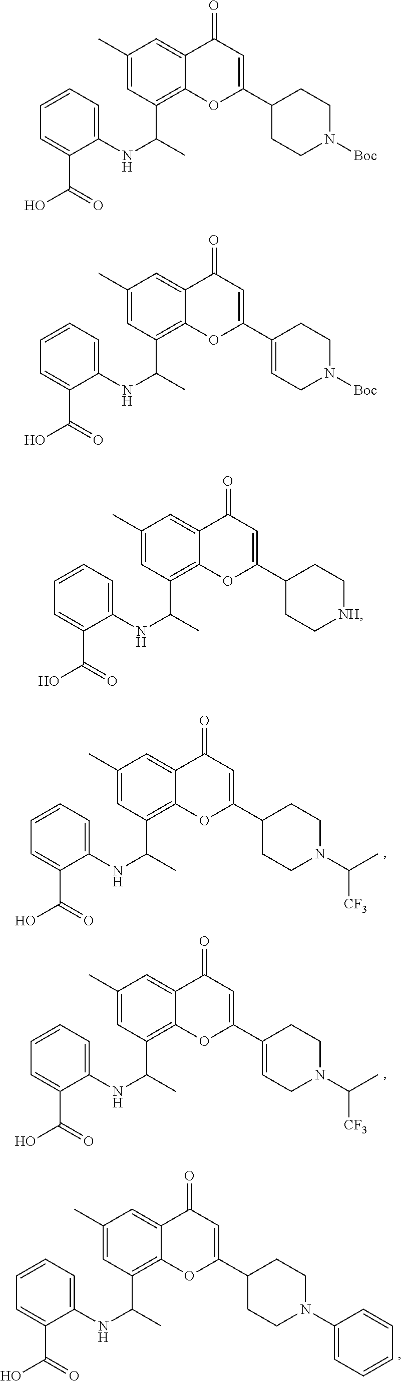 [SMILES: Cc1cc(C(C)Nc2ccccc2C(=O)O)c2oc(C3CCN(C(=O)OC(C)(C)C)CC3)cc(=O)c2c1 ; Cc1cc(C(C)Nc2ccccc2C(=O)O)c2oc(C3=CCN(C(=O)OC(C)(C)C)CC3)cc(=O)c2c1 ; Cc1cc(C(C)Nc2ccccc2C(=O)O)c2oc(C3CCNCC3)cc(=O)c2c1 ; Cc1cc(C(C)Nc2ccccc2C(=O)O)c2oc(C3CCN(C(C)C(F)(F)F)CC3)cc(=O)c2c1 ; Cc1cc(C(C)Nc2ccccc2C(=O)O)c2oc(C3=CCN(C(C)C(F)(F)F)CC3)cc(=O)c2c1 ; Cc1cc(C(C)Nc2ccccc2C(=O)O)c2oc(C3CCN(c4ccccc4)CC3)cc(=O)c2c1]

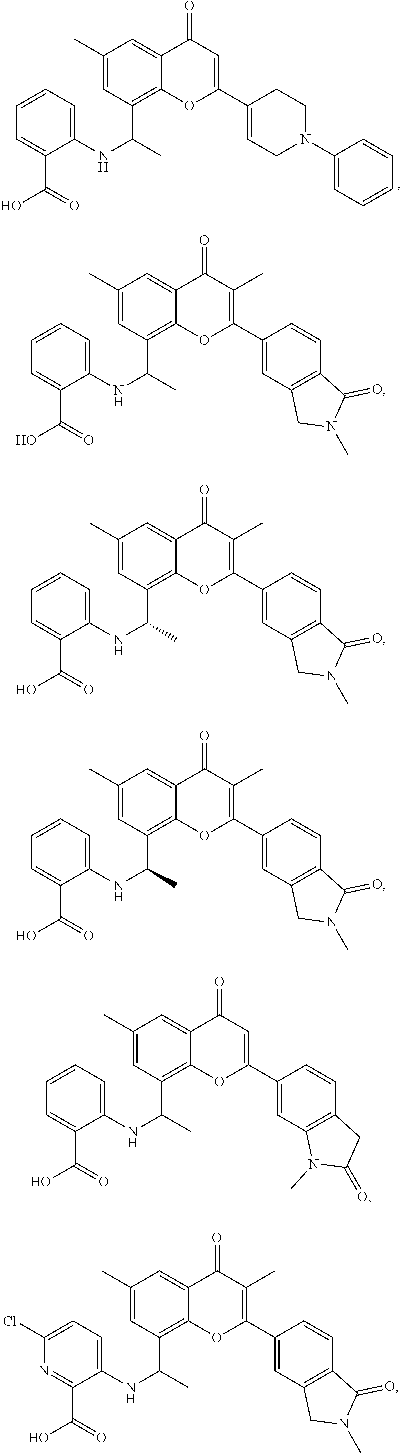 [SMILES: Cc1cc(C(C)Nc2ccccc2C(=O)O)c2oc(C3=CCN(c4ccccc4)CC3)cc(=O)c2c1 ; Cc1cc(C(C)Nc2ccccc2C(=O)O)c2oc(-c3ccc4c(c3)CN(C)C4=O)c(C)c(=O)c2c1 ; Cc1cc([C@H](C)Nc2ccccc2C(=O)O)c2oc(-c3ccc4c(c3)CN(C)C4=O)c(C)c(=O)c2c1 ; Cc1cc([C@@H](C)Nc2ccccc2C(=O)O)c2oc(-c3ccc4c(c3)CN(C)C4=O)c(C)c(=O)c2c1 ; Cc1cc(C(C)Nc2ccccc2C(=O)O)c2oc(-c3ccc4c(c3)N(C)C(=O)C4)cc(=O)c2c1 ; Cc1cc(C(C)Nc2ccc(Cl)nc2C(=O)O)c2oc(-c3ccc4c(c3)CN(C)C4=O)c(C)c(=O)c2c1]

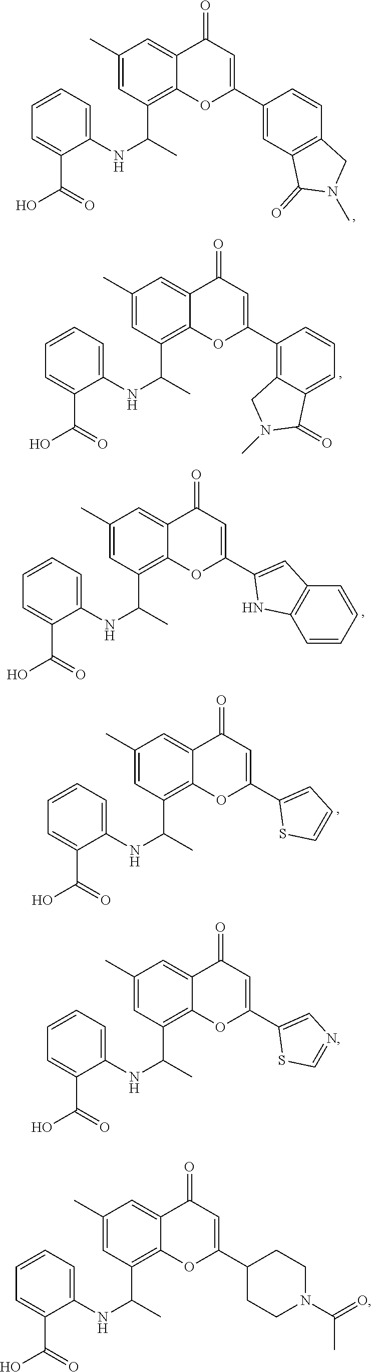 [SMILES: Cc1cc(C(C)Nc2ccccc2C(=O)O)c2oc(-c3ccc4c(c3)C(=O)N(C)C4)cc(=O)c2c1 ; Cc1cc(C(C)Nc2ccccc2C(=O)O)c2oc(-c3cccc4c3CN(C)C4=O)cc(=O)c2c1 ; Cc1cc(C(C)Nc2ccccc2C(=O)O)c2oc(-c3cc4ccccc4[nH]3)cc(=O)c2c1 ; Cc1cc(C(C)Nc2ccccc2C(=O)O)c2oc(-c3cccs3)cc(=O)c2c1 ; Cc1cc(C(C)Nc2ccccc2C(=O)O)c2oc(-c3cncs3)cc(=O)c2c1 ; CC(=O)N1CCC(c2cc(=O)c3cc(C)cc(C(C)Nc4ccccc4C(=O)O)c3o2)CC1]

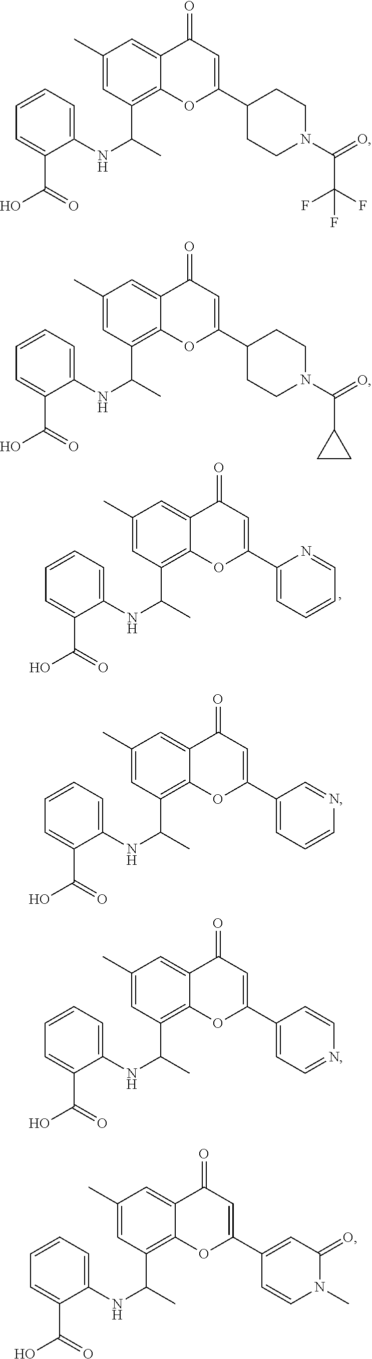 [SMILES: Cc1cc(C(C)Nc2ccccc2C(=O)O)c2oc(C3CCN(C(=O)C(F)(F)F)CC3)cc(=O)c2c1 ; Cc1cc(C(C)Nc2ccccc2C(=O)O)c2oc(C3CCN(C(=O)C4CC4)CC3)cc(=O)c2c1 ; Cc1cc(C(C)Nc2ccccc2C(=O)O)c2oc(-c3ccccn3)cc(=O)c2c1 ; Cc1cc(C(C)Nc2ccccc2C(=O)O)c2oc(-c3cccnc3)cc(=O)c2c1 ; Cc1cc(C(C)Nc2ccccc2C(=O)O)c2oc(-c3ccncc3)cc(=O)c2c1 ; Cc1cc(C(C)Nc2ccccc2C(=O)O)c2oc(-c3ccn(C)c(=O)c3)cc(=O)c2c1]

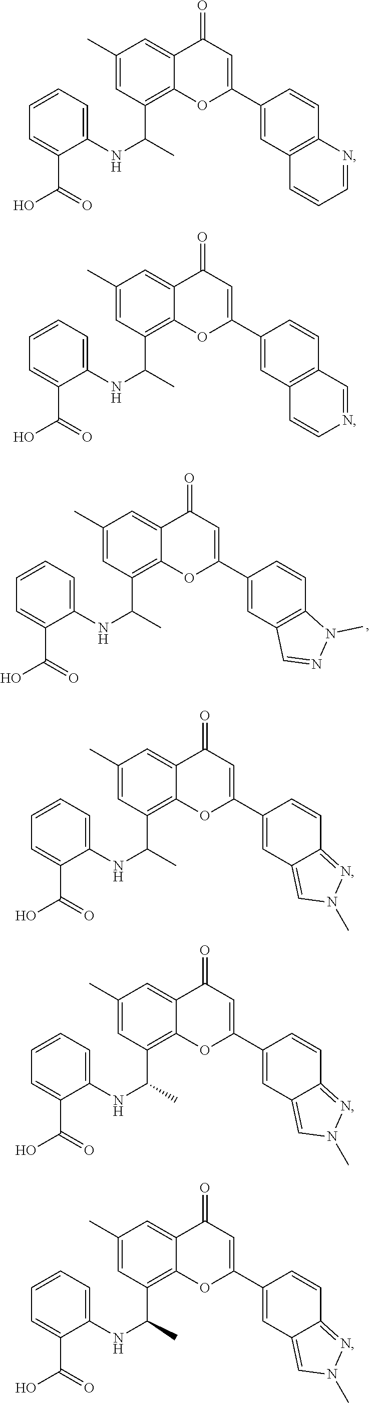 [SMILES: Cc1cc(C(C)Nc2ccccc2C(=O)O)c2oc(-c3ccc4ncccc4c3)cc(=O)c2c1 ; Cc1cc(C(C)Nc2ccccc2C(=O)O)c2oc(-c3ccc4cnccc4c3)cc(=O)c2c1 ; Cc1cc(C(C)Nc2ccccc2C(=O)O)c2oc(-c3ccc4c(cnn4C)c3)cc(=O)c2c1 ; Cc1cc(C(C)Nc2ccccc2C(=O)O)c2oc(-c3ccc4nn(C)cc4c3)cc(=O)c2c1 ; Cc1cc([C@H](C)Nc2ccccc2C(=O)O)c2oc(-c3ccc4nn(C)cc4c3)cc(=O)c2c1 ; Cc1cc([C@@H](C)Nc2ccccc2C(=O)O)c2oc(-c3ccc4nn(C)cc4c3)cc(=O)c2c1]

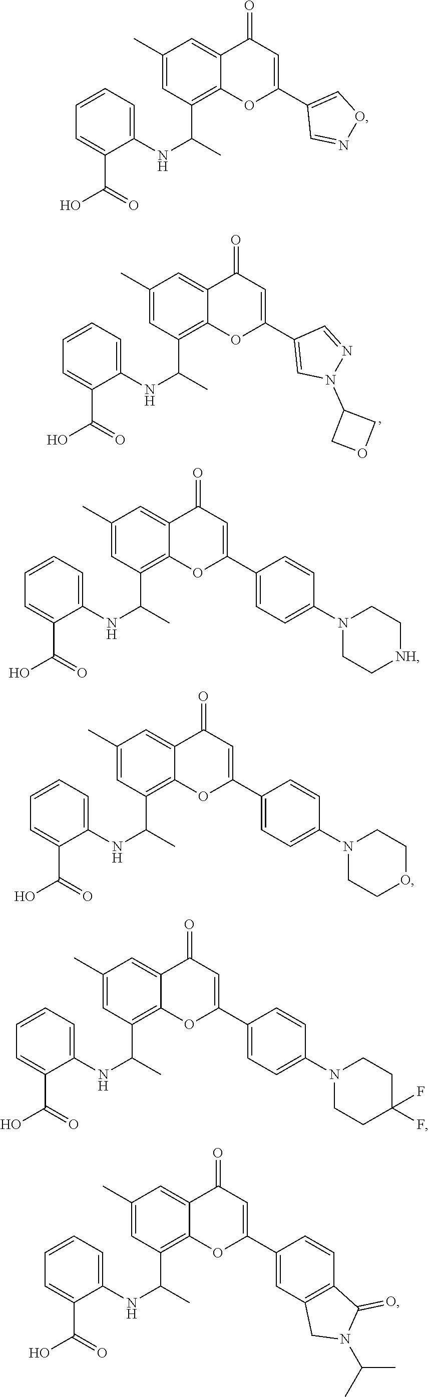 [SMILES: Cc1cc(C(C)Nc2ccccc2C(=O)O)c2oc(-c3cnoc3)cc(=O)c2c1 ; Cc1cc(C(C)Nc2ccccc2C(=O)O)c2oc(-c3cnn(C4COC4)c3)cc(=O)c2c1 ; Cc1cc(C(C)Nc2ccccc2C(=O)O)c2oc(-c3ccc(N4CCNCC4)cc3)cc(=O)c2c1 ; Cc1cc(C(C)Nc2ccccc2C(=O)O)c2oc(-c3ccc(N4CCOCC4)cc3)cc(=O)c2c1 ; Cc1cc(C(C)Nc2ccccc2C(=O)O)c2oc(-c3ccc(N4CCC(F)(F)CC4)cc3)cc(=O)c2c1 ; Cc1cc(C(C)Nc2ccccc2C(=O)O)c2oc(-c3ccc4c(c3)CN(C(C)C)C4=O)cc(=O)c2c1]

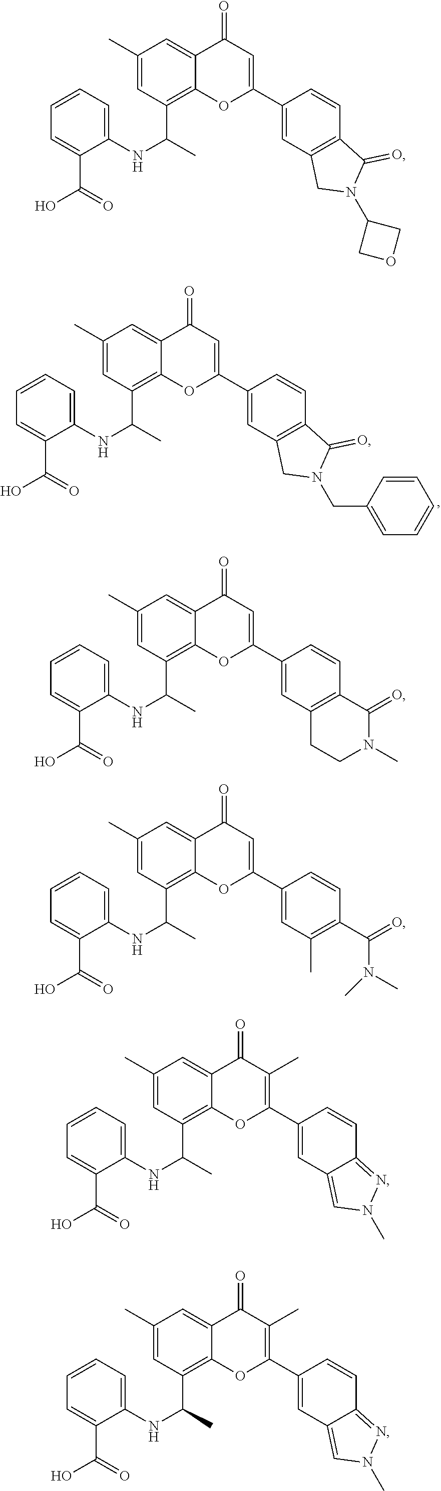 [SMILES: Cc1cc(C(C)Nc2ccccc2C(=O)O)c2oc(-c3ccc4c(c3)CN(C3COC3)C4=O)cc(=O)c2c1 ; Cc1cc(C(C)Nc2ccccc2C(=O)O)c2oc(-c3ccc4c(c3)CN(Cc3ccccc3)C4=O)cc(=O)c2c1 ; Cc1cc(C(C)Nc2ccccc2C(=O)O)c2oc(-c3ccc4c(c3)CCN(C)C4=O)cc(=O)c2c1 ; Cc1cc(C(C)Nc2ccccc2C(=O)O)c2oc(-c3ccc(C(=O)N(C)C)c(C)c3)cc(=O)c2c1 ; Cc1cc(C(C)Nc2ccccc2C(=O)O)c2oc(-c3ccc4nn(C)cc4c3)c(C)c(=O)c2c1 ; Cc1cc([C@@H](C)Nc2ccccc2C(=O)O)c2oc(-c3ccc4nn(C)cc4c3)c(C)c(=O)c2c1]

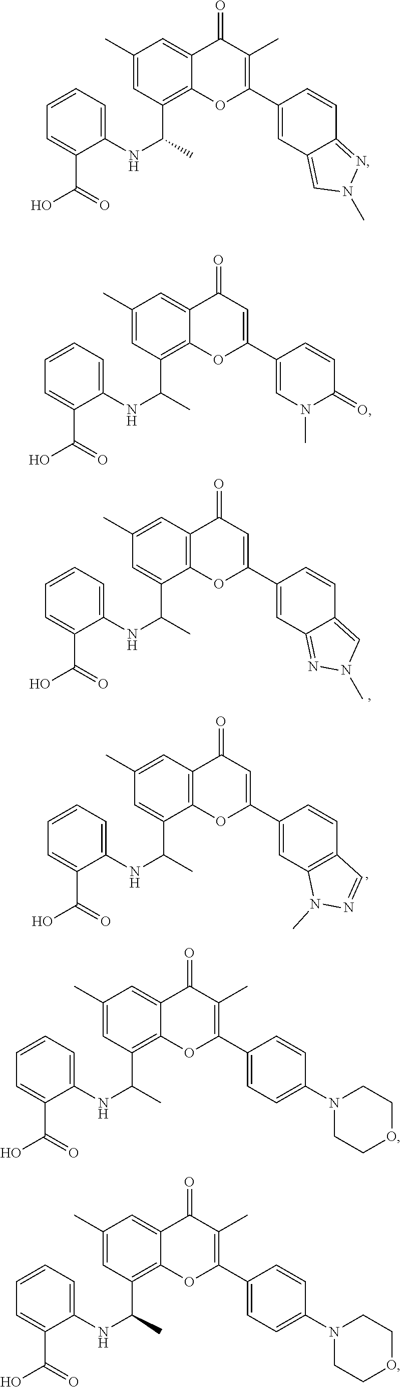 [SMILES: Cc1cc([C@H](C)Nc2ccccc2C(=O)O)c2oc(-c3ccc4nn(C)cc4c3)c(C)c(=O)c2c1 ; Cc1cc(C(C)Nc2ccccc2C(=O)O)c2oc(-c3ccc(=O)n(C)c3)cc(=O)c2c1 ; Cc1cc(C(C)Nc2ccccc2C(=O)O)c2oc(-c3ccc4cn(C)nc4c3)cc(=O)c2c1 ; Cc1cc(C(C)Nc2ccccc2C(=O)O)c2oc(-c3ccc4cnn(C)c4c3)cc(=O)c2c1 ; Cc1cc(C(C)Nc2ccccc2C(=O)O)c2oc(-c3ccc(N4CCOCC4)cc3)c(C)c(=O)c2c1 ; Cc1cc([C@@H](C)Nc2ccccc2C(=O)O)c2oc(-c3ccc(N4CCOCC4)cc3)c(C)c(=O)c2c1]

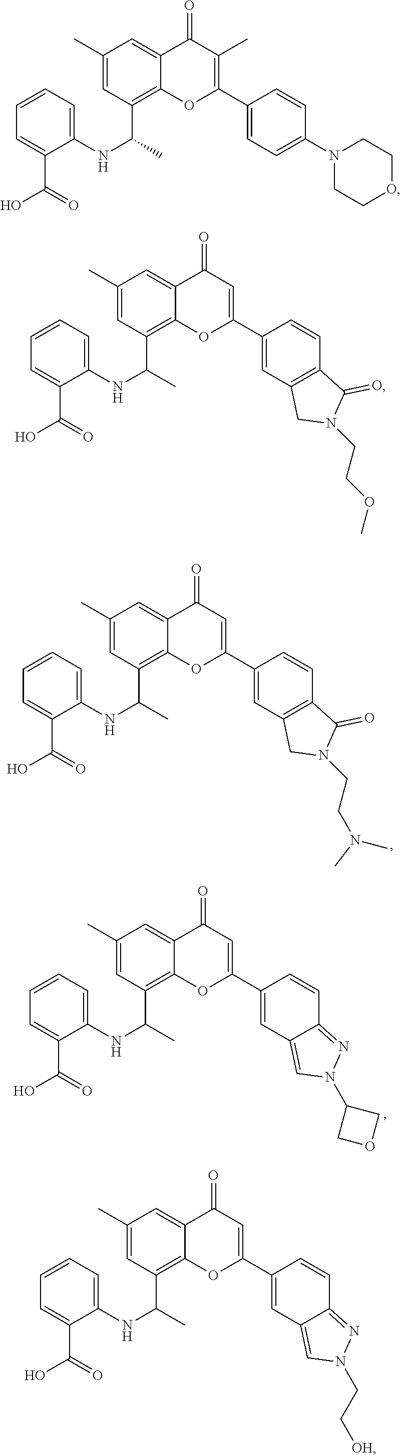 [SMILES: Cc1cc([C@H](C)Nc2ccccc2C(=O)O)c2oc(-c3ccc(N4CCOCC4)cc3)c(C)c(=O)c2c1 ; COCCN1Cc2cc(-c3cc(=O)c4cc(C)cc(C(C)Nc5ccccc5C(=O)O)c4o3)ccc2C1=O ; Cc1cc(C(C)Nc2ccccc2C(=O)O)c2oc(-c3ccc4c(c3)CN(CCN(C)C)C4=O)cc(=O)c2c1 ; Cc1cc(C(C)Nc2ccccc2C(=O)O)c2oc(-c3ccc4nn(C5COC5)cc4c3)cc(=O)c2c1 ; Cc1cc(C(C)Nc2ccccc2C(=O)O)c2oc(-c3ccc4nn(CCO)cc4c3)cc(=O)c2c1]

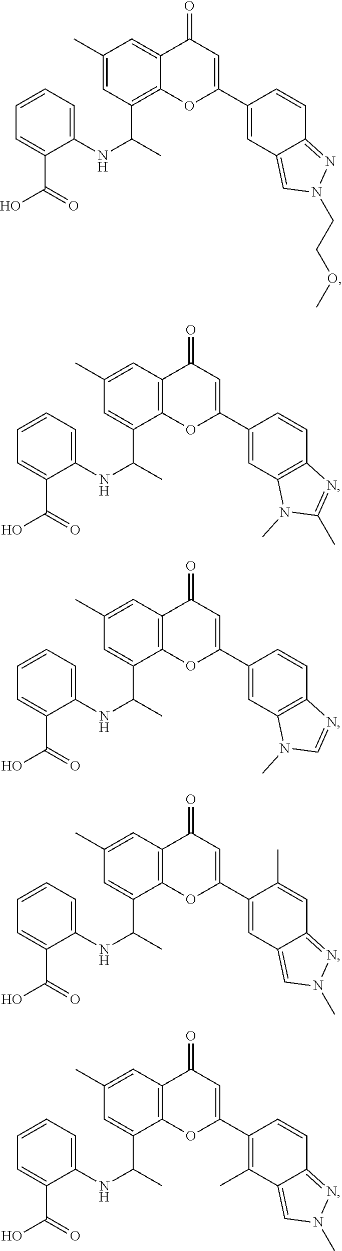 [SMILES: COCCn1cc2cc(-c3cc(=O)c4cc(C)cc(C(C)Nc5ccccc5C(=O)O)c4o3)ccc2n1 ; Cc1cc(C(C)Nc2ccccc2C(=O)O)c2oc(-c3ccc4nc(C)n(C)c4c3)cc(=O)c2c1 ; Cc1cc(C(C)Nc2ccccc2C(=O)O)c2oc(-c3ccc4ncn(C)c4c3)cc(=O)c2c1 ; Cc1cc(C(C)Nc2ccccc2C(=O)O)c2oc(-c3cc4cn(C)nc4cc3C)cc(=O)c2c1 ; Cc1cc(C(C)Nc2ccccc2C(=O)O)c2oc(-c3ccc4nn(C)cc4c3C)cc(=O)c2c1]

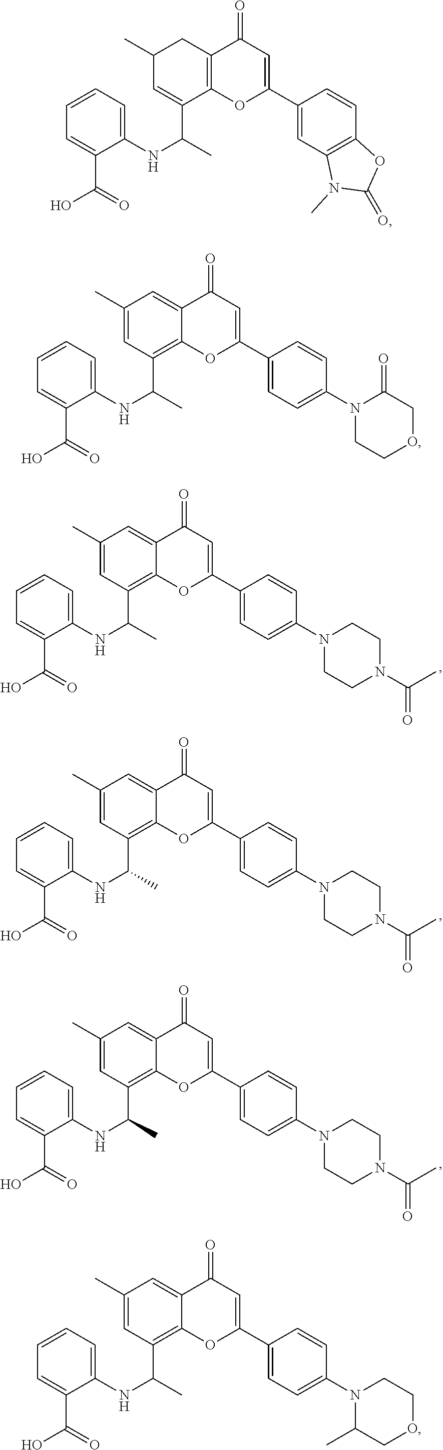 [SMILES: CC1C=C(C(C)Nc2ccccc2C(=O)O)c2oc(-c3ccc4oc(=O)n(C)c4c3)cc(=O)c2C1 ; Cc1cc(C(C)Nc2ccccc2C(=O)O)c2oc(-c3ccc(N4CCOCC4=O)cc3)cc(=O)c2c1 ; CC(=O)N1CCN(c2ccc(-c3cc(=O)c4cc(C)cc(C(C)Nc5ccccc5C(=O)O)c4o3)cc2)CC1 ; CC(=O)N1CCN(c2ccc(-c3cc(=O)c4cc(C)cc([C@H](C)Nc5ccccc5C(=O)O)c4o3)cc2)CC1 ; CC(=O)N1CCN(c2ccc(-c3cc(=O)c4cc(C)cc([C@@H](C)Nc5ccccc5C(=O)O)c4o3)cc2)CC1 ; Cc1cc(C(C)Nc2ccccc2C(=O)O)c2oc(-c3ccc(N4CCOCC4C)cc3)cc(=O)c2c1]

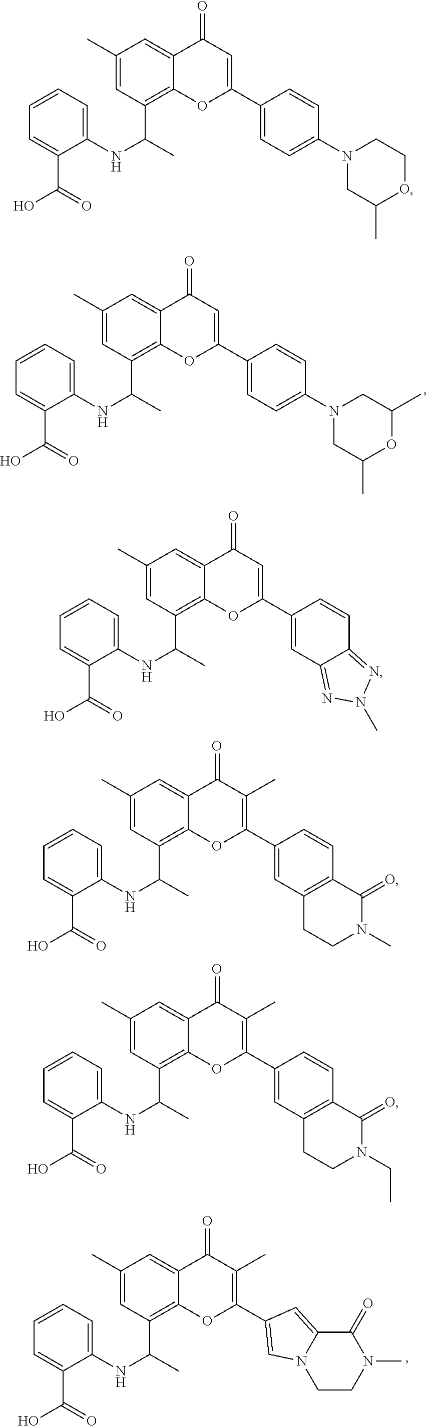 [SMILES: Cc1cc(C(C)Nc2ccccc2C(=O)O)c2oc(-c3ccc(N4CCOC(C)C4)cc3)cc(=O)c2c1 ; Cc1cc(C(C)Nc2ccccc2C(=O)O)c2oc(-c3ccc(N4CC(C)OC(C)C4)cc3)cc(=O)c2c1 ; Cc1cc(C(C)Nc2ccccc2C(=O)O)c2oc(-c3ccc4nn(C)nc4c3)cc(=O)c2c1 ; Cc1cc(C(C)Nc2ccccc2C(=O)O)c2oc(-c3ccc4c(c3)CCN(C)C4=O)c(C)c(=O)c2c1 ; CCN1CCc2cc(-c3oc4c(C(C)Nc5ccccc5C(=O)O)cc(C)cc4c(=O)c3C)ccc2C1=O ; Cc1cc(C(C)Nc2ccccc2C(=O)O)c2oc(-c3cc4n(c3)CCN(C)C4=O)c(C)c(=O)c2c1]

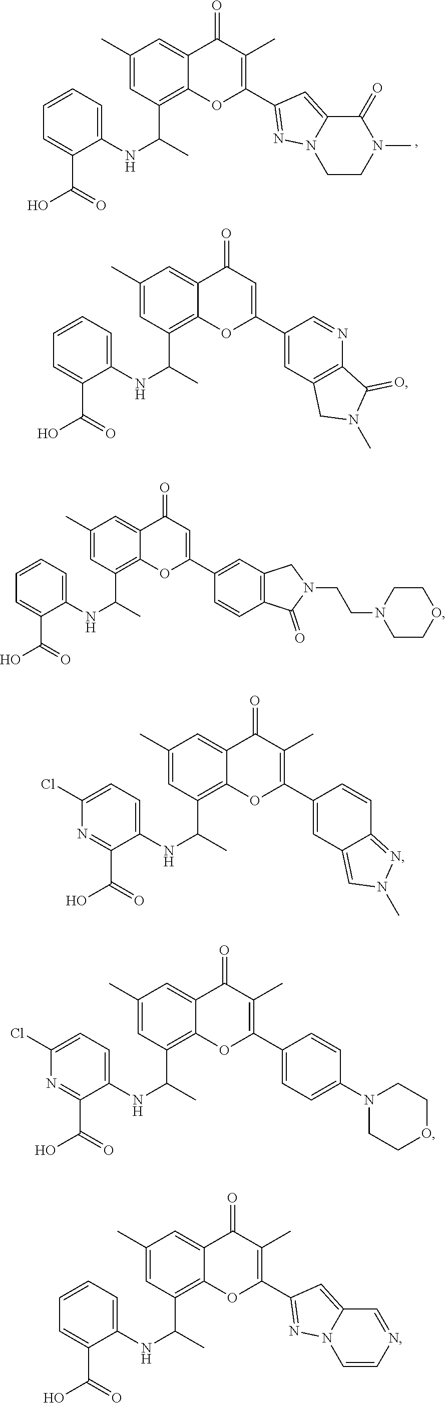 [SMILES: Cc1cc(C(C)Nc2ccccc2C(=O)O)c2oc(-c3cc4n(n3)CCN(C)C4=O)c(C)c(=O)c2c1 ; Cc1cc(C(C)Nc2ccccc2C(=O)O)c2oc(-c3cnc4c(c3)CN(C)C4=O)cc(=O)c2c1 ; Cc1cc(C(C)Nc2ccccc2C(=O)O)c2oc(-c3ccc4c(c3)CN(CCN3CCOCC3)C4=O)cc(=O)c2c1 ; Cc1cc(C(C)Nc2ccc(Cl)nc2C(=O)O)c2oc(-c3ccc4nn(C)cc4c3)c(C)c(=O)c2c1 ; Cc1cc(C(C)Nc2ccc(Cl)nc2C(=O)O)c2oc(-c3ccc(N4CCOCC4)cc3)c(C)c(=O)c2c1 ; Cc1cc(C(C)Nc2ccccc2C(=O)O)c2oc(-c3cc4cnccn4n3)c(C)c(=O)c2c1]

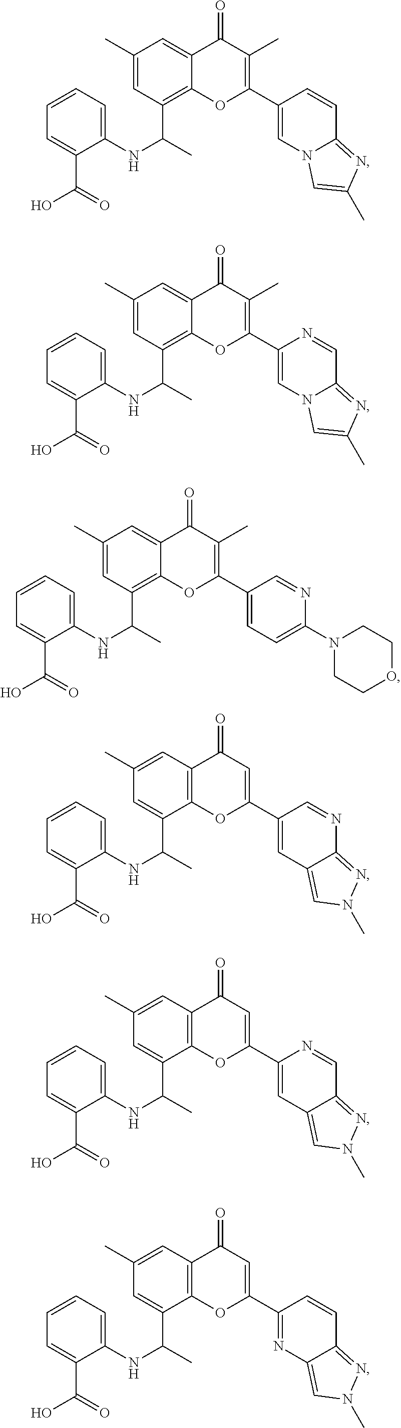 [SMILES: Cc1cc(C(C)Nc2ccccc2C(=O)O)c2oc(-c3ccc4nc(C)cn4c3)c(C)c(=O)c2c1 ; Cc1cc(C(C)Nc2ccccc2C(=O)O)c2oc(-c3cn4cc(C)nc4cn3)c(C)c(=O)c2c1 ; Cc1cc(C(C)Nc2ccccc2C(=O)O)c2oc(-c3ccc(N4CCOCC4)nc3)c(C)c(=O)c2c1 ; Cc1cc(C(C)Nc2ccccc2C(=O)O)c2oc(-c3cnc4nn(C)cc4c3)cc(=O)c2c1 ; Cc1cc(C(C)Nc2ccccc2C(=O)O)c2oc(-c3cc4cn(C)nc4cn3)cc(=O)c2c1 ; Cc1cc(C(C)Nc2ccccc2C(=O)O)c2oc(-c3ccc4nn(C)cc4n3)cc(=O)c2c1]

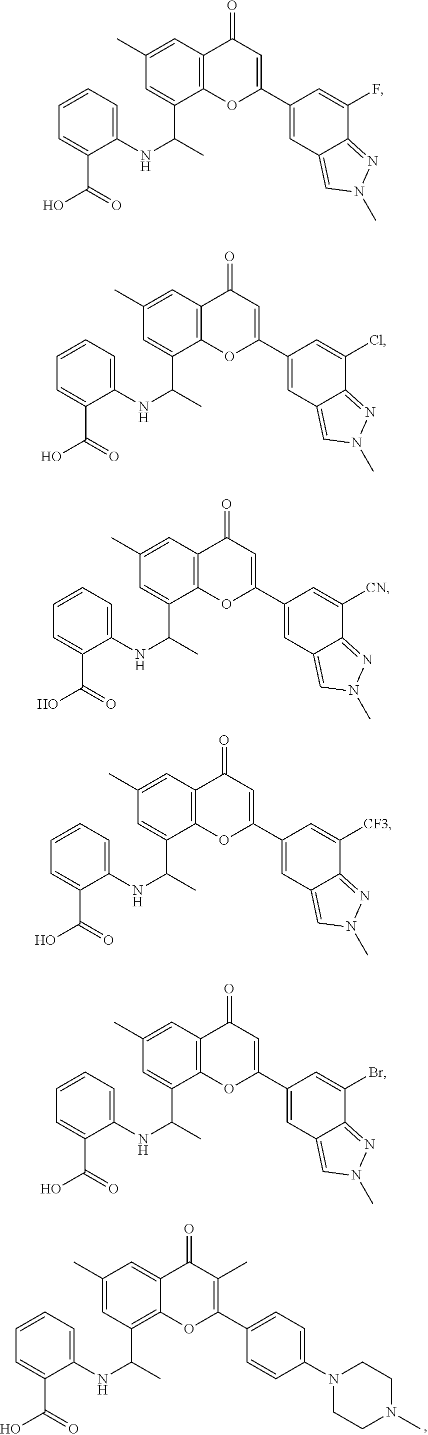 [SMILES: Cc1cc(C(C)Nc2ccccc2C(=O)O)c2oc(-c3cc(F)c4nn(C)cc4c3)cc(=O)c2c1 ; Cc1cc(C(C)Nc2ccccc2C(=O)O)c2oc(-c3cc(Cl)c4nn(C)cc4c3)cc(=O)c2c1 ; Cc1cc(C(C)Nc2ccccc2C(=O)O)c2oc(-c3cc(C#N)c4nn(C)cc4c3)cc(=O)c2c1 ; Cc1cc(C(C)Nc2ccccc2C(=O)O)c2oc(-c3cc(C(F)(F)F)c4nn(C)cc4c3)cc(=O)c2c1 ; Cc1cc(C(C)Nc2ccccc2C(=O)O)c2oc(-c3cc(Br)c4nn(C)cc4c3)cc(=O)c2c1 ; Cc1cc(C(C)Nc2ccccc2C(=O)O)c2oc(-c3ccc(N4CCN(C)CC4)cc3)c(C)c(=O)c2c1]

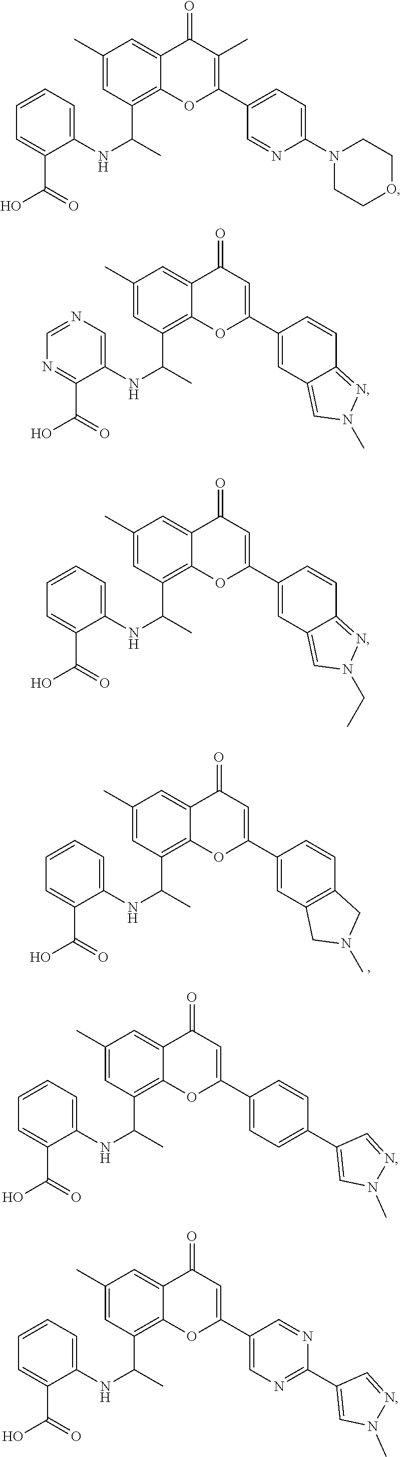 [SMILES: Cc1cc(C(C)Nc2ccccc2C(=O)O)c2oc(-c3ccc(N4CCOCC4)nc3)c(C)c(=O)c2c1 ; Cc1cc(C(C)Nc2cncnc2C(=O)O)c2oc(-c3ccc4nn(C)cc4c3)cc(=O)c2c1 ; CCn1cc2cc(-c3cc(=O)c4cc(C)cc(C(C)Nc5ccccc5C(=O)O)c4o3)ccc2n1 ; Cc1cc(C(C)Nc2ccccc2C(=O)O)c2oc(-c3ccc4c(c3)CN(C)C4)cc(=O)c2c1 ; Cc1cc(C(C)Nc2ccccc2C(=O)O)c2oc(-c3ccc(-c4cnn(C)c4)cc3)cc(=O)c2c1 ; Cc1cc(C(C)Nc2ccccc2C(=O)O)c2oc(-c3cnc(-c4cnn(C)c4)nc3)cc(=O)c2c1]

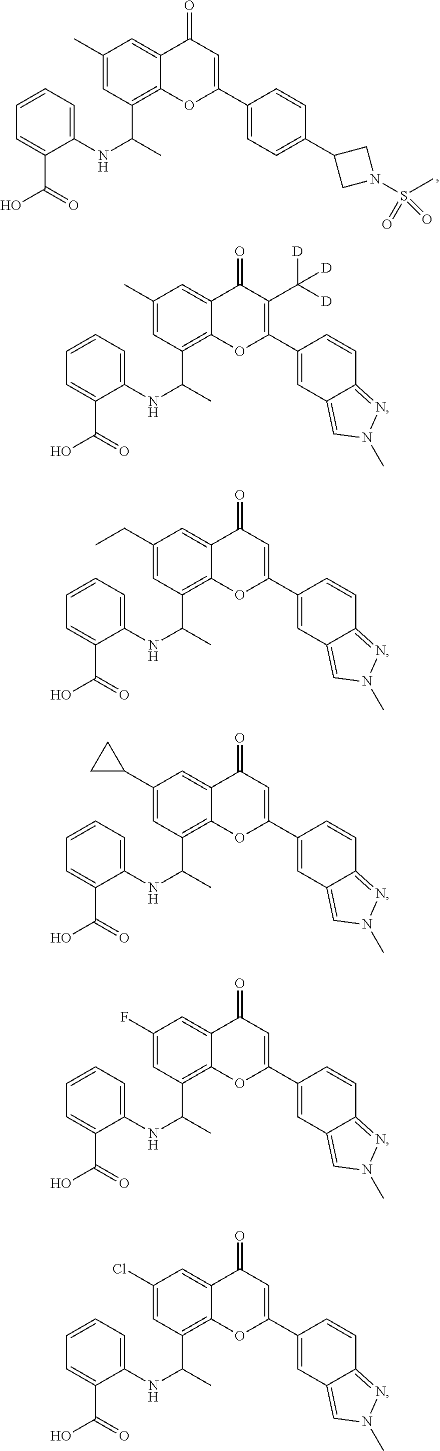 [SMILES: Cc1cc(C(C)Nc2ccccc2C(=O)O)c2oc(-c3ccc(C4CN(S(C)(=O)=O)C4)cc3)cc(=O)c2c1 ; [2H]C([2H])([2H])c1c(-c2ccc3nn(C)cc3c2)oc2c(C(C)Nc3ccccc3C(=O)O)cc(C)cc2c1=O ; CCc1cc(C(C)Nc2ccccc2C(=O)O)c2oc(-c3ccc4nn(C)cc4c3)cc(=O)c2c1 ; CC(Nc1ccccc1C(=O)O)c1cc(C2CC2)cc2c(=O)cc(-c3ccc4nn(C)cc4c3)oc12 ; CC(Nc1ccccc1C(=O)O)c1cc(F)cc2c(=O)cc(-c3ccc4nn(C)cc4c3)oc12 ; CC(Nc1ccccc1C(=O)O)c1cc(Cl)cc2c(=O)cc(-c3ccc4nn(C)cc4c3)oc12]

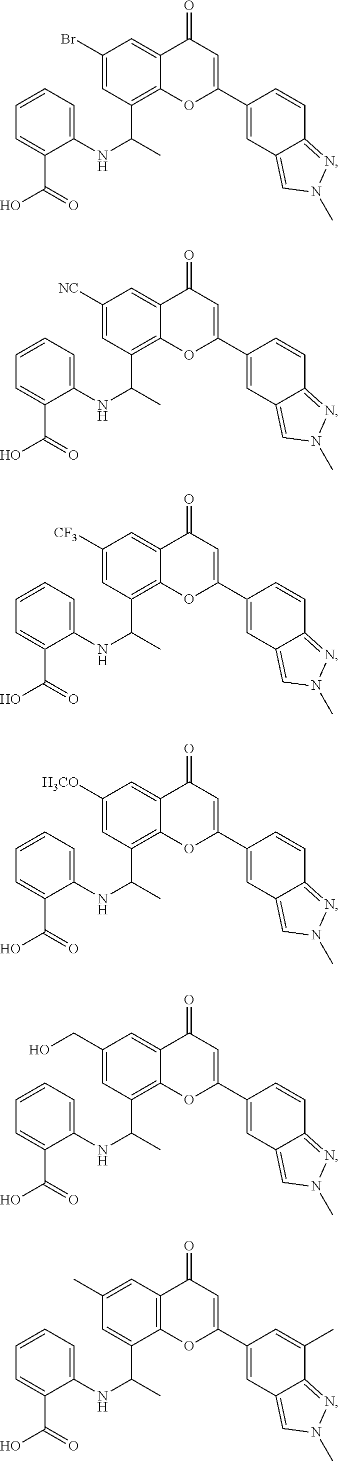 [SMILES: CC(Nc1ccccc1C(=O)O)c1cc(Br)cc2c(=O)cc(-c3ccc4nn(C)cc4c3)oc12 ; CC(Nc1ccccc1C(=O)O)c1cc(C#N)cc2c(=O)cc(-c3ccc4nn(C)cc4c3)oc12 ; CC(Nc1ccccc1C(=O)O)c1cc(C(F)(F)F)cc2c(=O)cc(-c3ccc4nn(C)cc4c3)oc12 ; COc1cc(C(C)Nc2ccccc2C(=O)O)c2oc(-c3ccc4nn(C)cc4c3)cc(=O)c2c1 ; CC(Nc1ccccc1C(=O)O)c1cc(CO)cc2c(=O)cc(-c3ccc4nn(C)cc4c3)oc12 ; Cc1cc(C(C)Nc2ccccc2C(=O)O)c2oc(-c3cc(C)c4nn(C)cc4c3)cc(=O)c2c1]

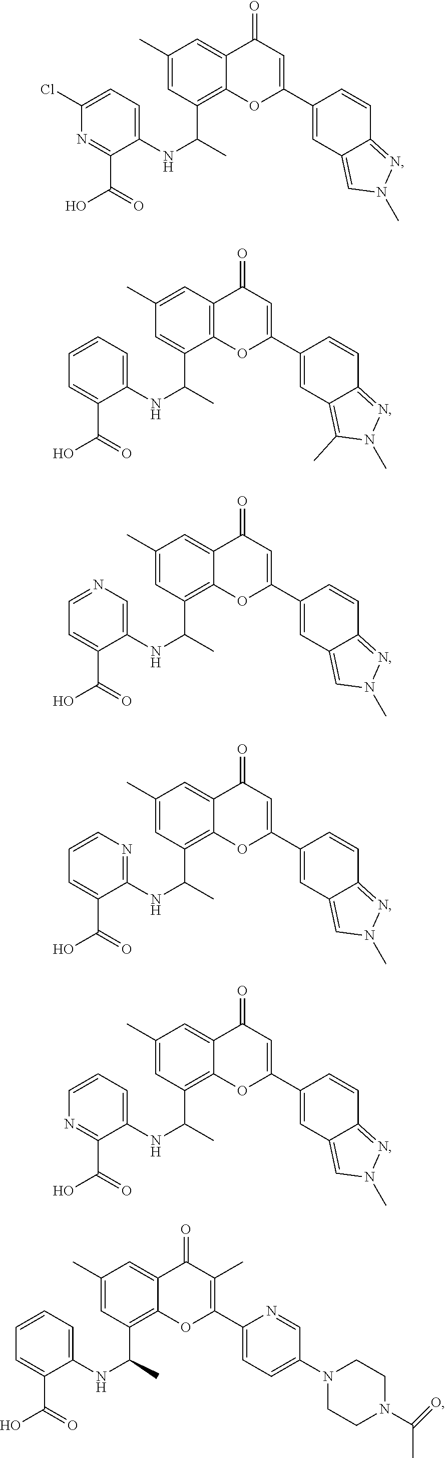 [SMILES: Cc1cc(C(C)Nc2ccc(Cl)nc2C(=O)O)c2oc(-c3ccc4nn(C)cc4c3)cc(=O)c2c1 ; Cc1cc(C(C)Nc2ccccc2C(=O)O)c2oc(-c3ccc4nn(C)c(C)c4c3)cc(=O)c2c1 ; Cc1cc(C(C)Nc2cnccc2C(=O)O)c2oc(-c3ccc4nn(C)cc4c3)cc(=O)c2c1 ; Cc1cc(C(C)Nc2ncccc2C(=O)O)c2oc(-c3ccc4nn(C)cc4c3)cc(=O)c2c1 ; Cc1cc(C(C)Nc2cccnc2C(=O)O)c2oc(-c3ccc4nn(C)cc4c3)cc(=O)c2c1 ; CC(=O)N1CCN(c2ccc(-c3oc4c([C@@H](C)Nc5ccccc5C(=O)O)cc(C)cc4c(=O)c3C)nc2)CC1]

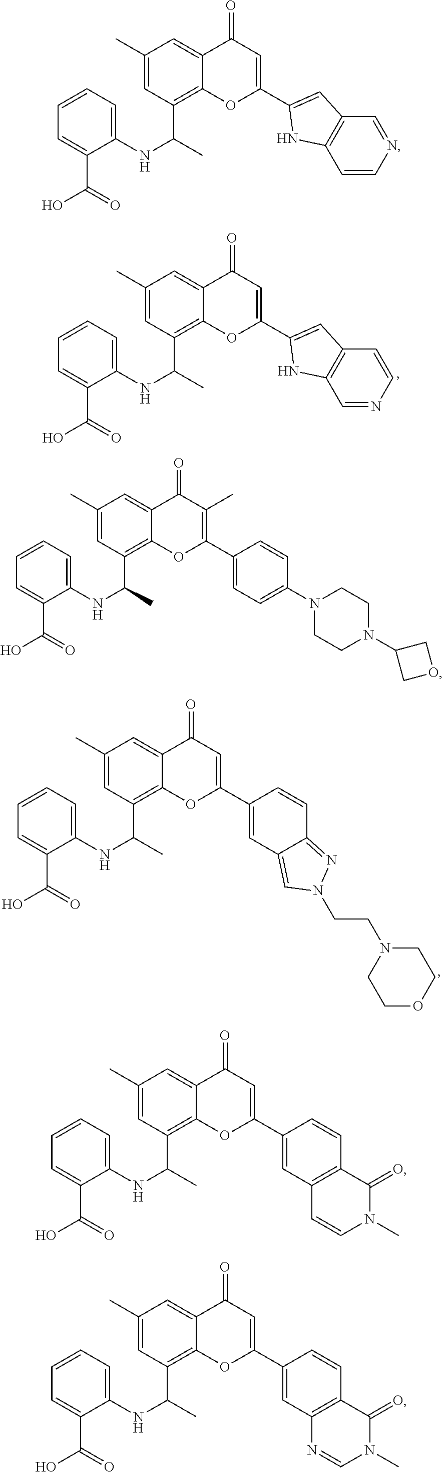 [SMILES: Cc1cc(C(C)Nc2ccccc2C(=O)O)c2oc(-c3cc4cnccc4[nH]3)cc(=O)c2c1 ; Cc1cc(C(C)Nc2ccccc2C(=O)O)c2oc(-c3cc4ccncc4[nH]3)cc(=O)c2c1 ; Cc1cc([C@@H](C)Nc2ccccc2C(=O)O)c2oc(-c3ccc(N4CCN(C5COC5)CC4)cc3)c(C)c(=O)c2c1 ; Cc1cc(C(C)Nc2ccccc2C(=O)O)c2oc(-c3ccc4nn(CCN5CCOCC5)cc4c3)cc(=O)c2c1 ; Cc1cc(C(C)Nc2ccccc2C(=O)O)c2oc(-c3ccc4c(=O)n(C)ccc4c3)cc(=O)c2c1 ; Cc1cc(C(C)Nc2ccccc2C(=O)O)c2oc(-c3ccc4c(=O)n(C)cnc4c3)cc(=O)c2c1]

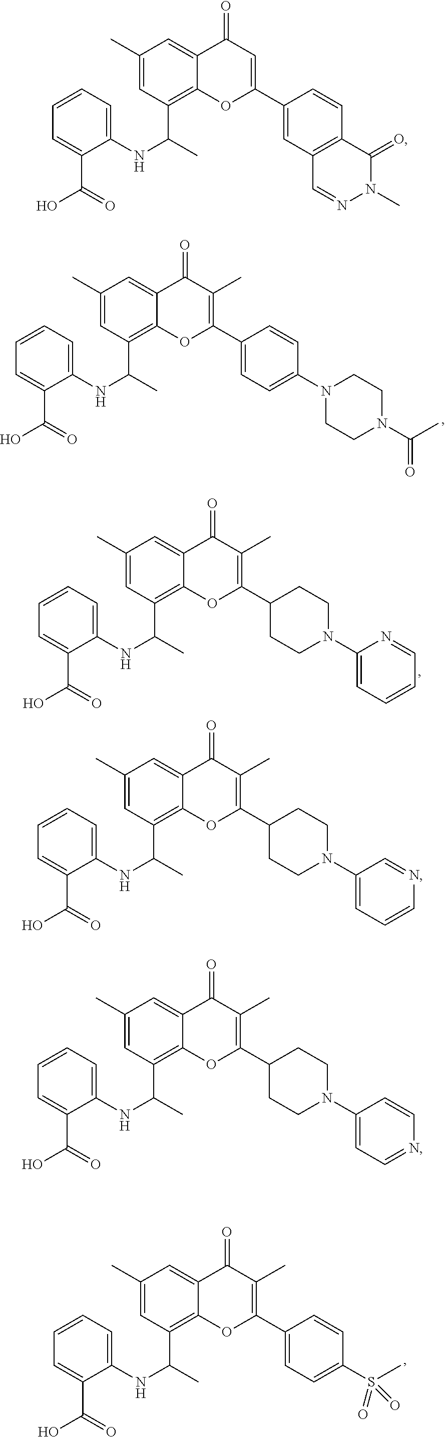 [SMILES: Cc1cc(C(C)Nc2ccccc2C(=O)O)c2oc(-c3ccc4c(=O)n(C)ncc4c3)cc(=O)c2c1 ; CC(=O)N1CCN(c2ccc(-c3oc4c(C(C)Nc5ccccc5C(=O)O)cc(C)cc4c(=O)c3C)cc2)CC1 ; Cc1cc(C(C)Nc2ccccc2C(=O)O)c2oc(C3CCN(c4ccccn4)CC3)c(C)c(=O)c2c1 ; Cc1cc(C(C)Nc2ccccc2C(=O)O)c2oc(C3CCN(c4cccnc4)CC3)c(C)c(=O)c2c1 ; Cc1cc(C(C)Nc2ccccc2C(=O)O)c2oc(C3CCN(c4ccncc4)CC3)c(C)c(=O)c2c1 ; Cc1cc(C(C)Nc2ccccc2C(=O)O)c2oc(-c3ccc(S(C)(=O)=O)cc3)c(C)c(=O)c2c1]

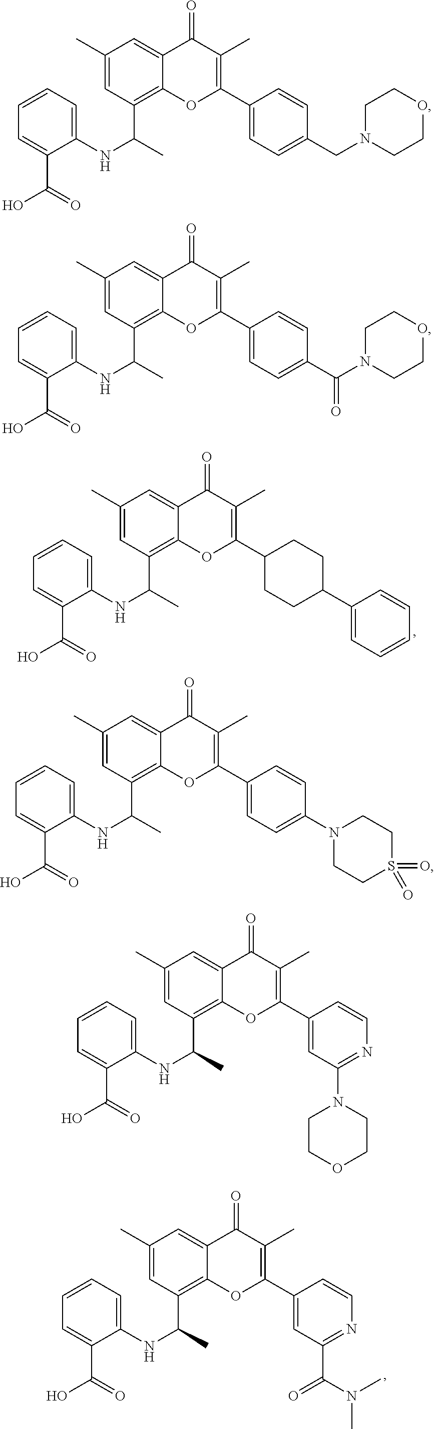 [SMILES: Cc1cc(C(C)Nc2ccccc2C(=O)O)c2oc(-c3ccc(CN4CCOCC4)cc3)c(C)c(=O)c2c1 ; Cc1cc(C(C)Nc2ccccc2C(=O)O)c2oc(-c3ccc(C(=O)N4CCOCC4)cc3)c(C)c(=O)c2c1 ; Cc1cc(C(C)Nc2ccccc2C(=O)O)c2oc(C3CCC(c4ccccc4)CC3)c(C)c(=O)c2c1 ; Cc1cc(C(C)Nc2ccccc2C(=O)O)c2oc(-c3ccc(N4CCS(=O)(=O)CC4)cc3)c(C)c(=O)c2c1 ; Cc1cc([C@@H](C)Nc2ccccc2C(=O)O)c2oc(-c3ccnc(N4CCOCC4)c3)c(C)c(=O)c2c1 ; Cc1cc([C@@H](C)Nc2ccccc2C(=O)O)c2oc(-c3ccnc(C(=O)N(C)C)c3)c(C)c(=O)c2c1]

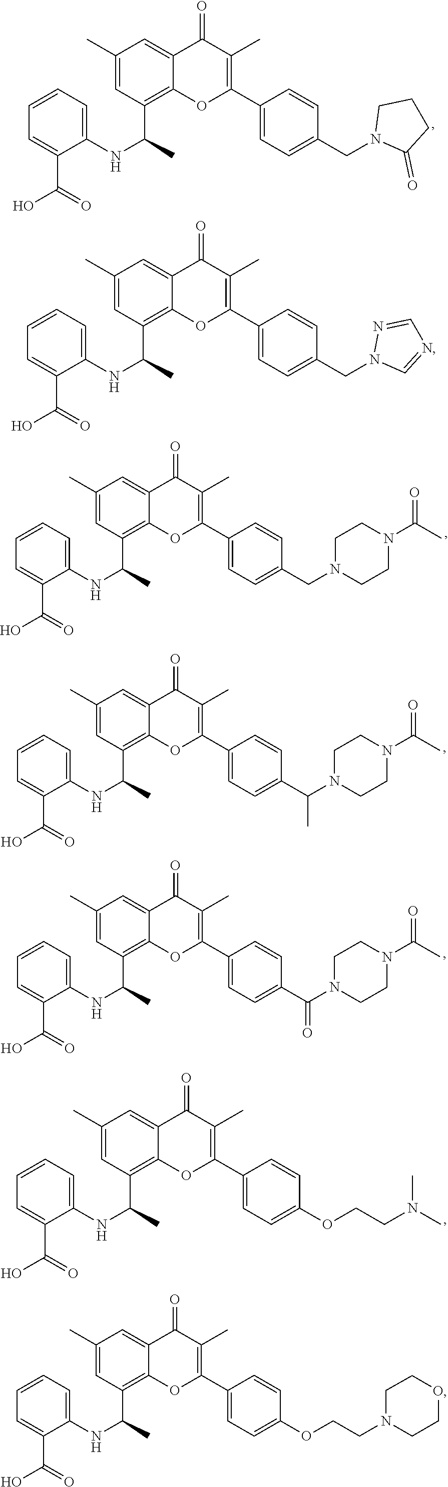

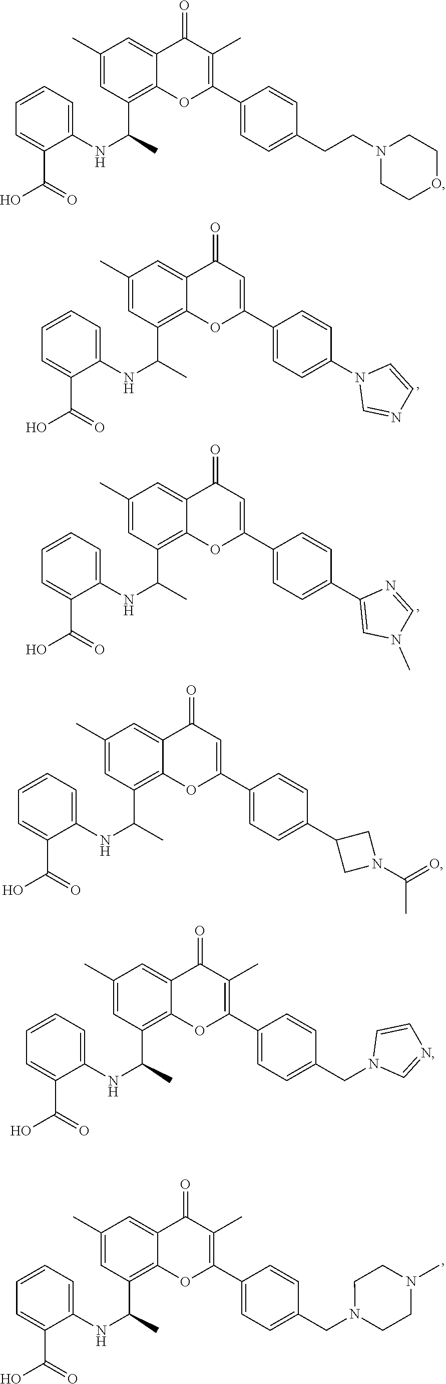 [SMILES: Cc1cc([C@@H](C)Nc2ccccc2C(=O)O)c2oc(-c3ccc(CCN4CCOCC4)cc3)c(C)c(=O)c2c1 ; Cc1cc(C(C)Nc2ccccc2C(=O)O)c2oc(-c3ccc(-n4ccnc4)cc3)cc(=O)c2c1 ; Cc1cc(C(C)Nc2ccccc2C(=O)O)c2oc(-c3ccc(-c4cn(C)cn4)cc3)cc(=O)c2c1 ; CC(=O)N1CC(c2ccc(-c3cc(=O)c4cc(C)cc(C(C)Nc5ccccc5C(=O)O)c4o3)cc2)C1 ; Cc1cc([C@@H](C)Nc2ccccc2C(=O)O)c2oc(-c3ccc(Cn4ccnc4)cc3)c(C)c(=O)c2c1 ; Cc1cc([C@@H](C)Nc2ccccc2C(=O)O)c2oc(-c3ccc(CN4CCN(C)CC4)cc3)c(C)c(=O)c2c1]

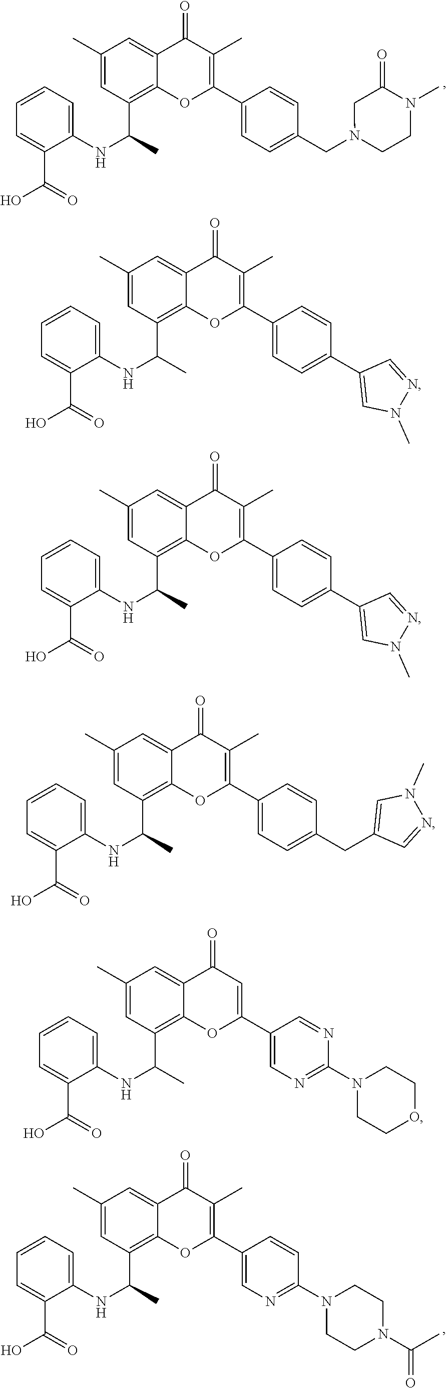 [SMILES: Cc1cc([C@@H](C)Nc2ccccc2C(=O)O)c2oc(-c3ccc(CN4CCN(C)C(=O)C4)cc3)c(C)c(=O)c2c1 ; Cc1cc(C(C)Nc2ccccc2C(=O)O)c2oc(-c3ccc(-c4cnn(C)c4)cc3)c(C)c(=O)c2c1 ; Cc1cc([C@@H](C)Nc2ccccc2C(=O)O)c2oc(-c3ccc(-c4cnn(C)c4)cc3)c(C)c(=O)c2c1 ; Cc1cc([C@@H](C)Nc2ccccc2C(=O)O)c2oc(-c3ccc(Cc4cnn(C)c4)cc3)c(C)c(=O)c2c1 ; Cc1cc(C(C)Nc2ccccc2C(=O)O)c2oc(-c3cnc(N4CCOCC4)nc3)cc(=O)c2c1 ; CC(=O)N1CCN(c2ccc(-c3oc4c([C@@H](C)Nc5ccccc5C(=O)O)cc(C)cc4c(=O)c3C)cn2)CC1]

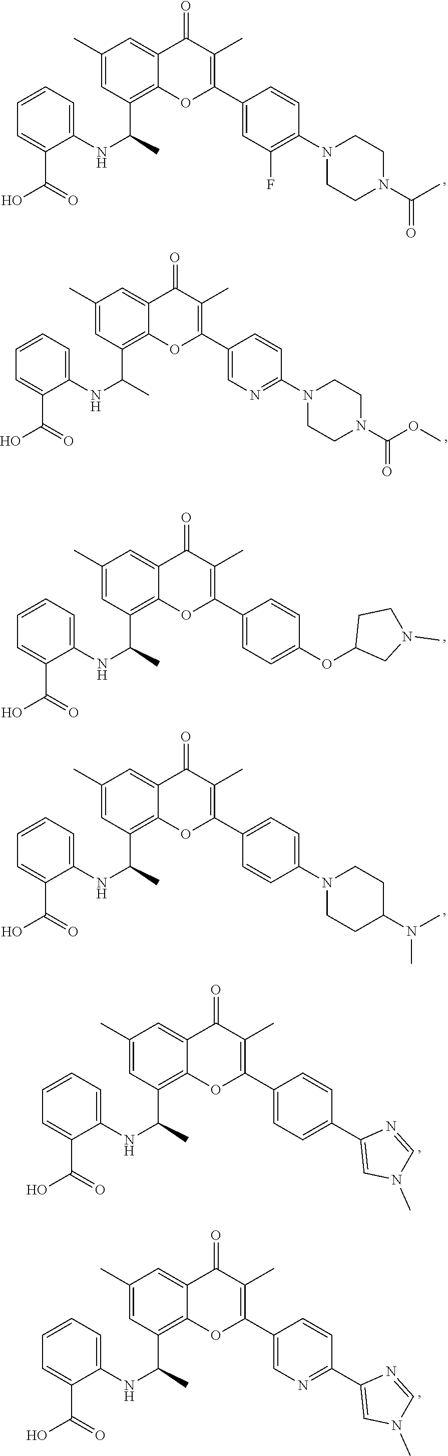 [SMILES: CC(=O)N1CCN(c2ccc(-c3oc4c([C@@H](C)Nc5ccccc5C(=O)O)cc(C)cc4c(=O)c3C)cc2F)CC1 ; COC(=O)N1CCN(c2ccc(-c3oc4c(C(C)Nc5ccccc5C(=O)O)cc(C)cc4c(=O)c3C)cn2)CC1 ; Cc1cc([C@@H](C)Nc2ccccc2C(=O)O)c2oc(-c3ccc(OC4CCN(C)C4)cc3)c(C)c(=O)c2c1 ; Cc1cc([C@@H](C)Nc2ccccc2C(=O)O)c2oc(-c3ccc(N4CCC(N(C)C)CC4)cc3)c(C)c(=O)c2c1 ; Cc1cc([C@@H](C)Nc2ccccc2C(=O)O)c2oc(-c3ccc(-c4cn(C)cn4)cc3)c(C)c(=O)c2c1 ; Cc1cc([C@@H](C)Nc2ccccc2C(=O)O)c2oc(-c3ccc(-c4cn(C)cn4)nc3)c(C)c(=O)c2c1]

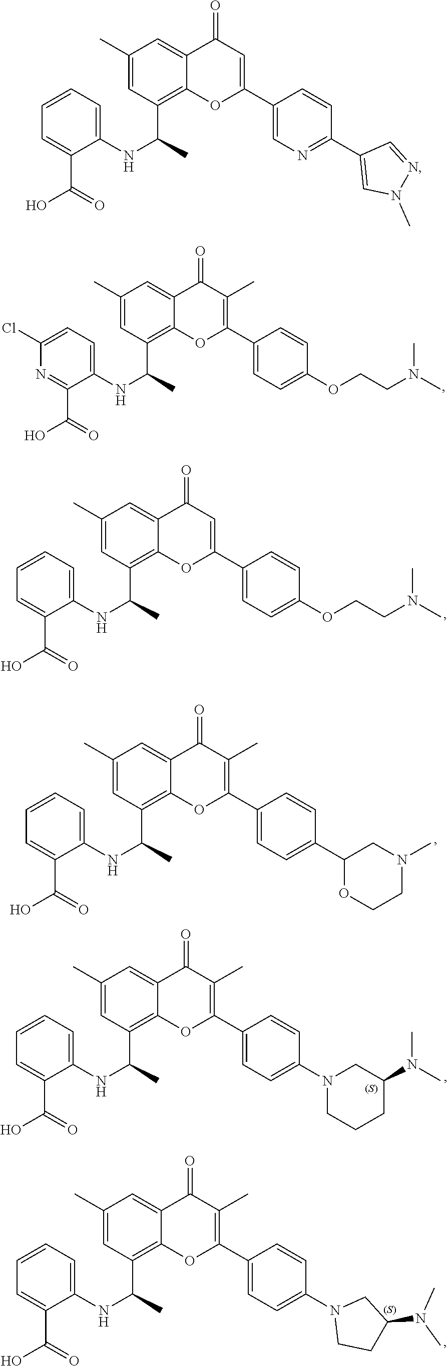

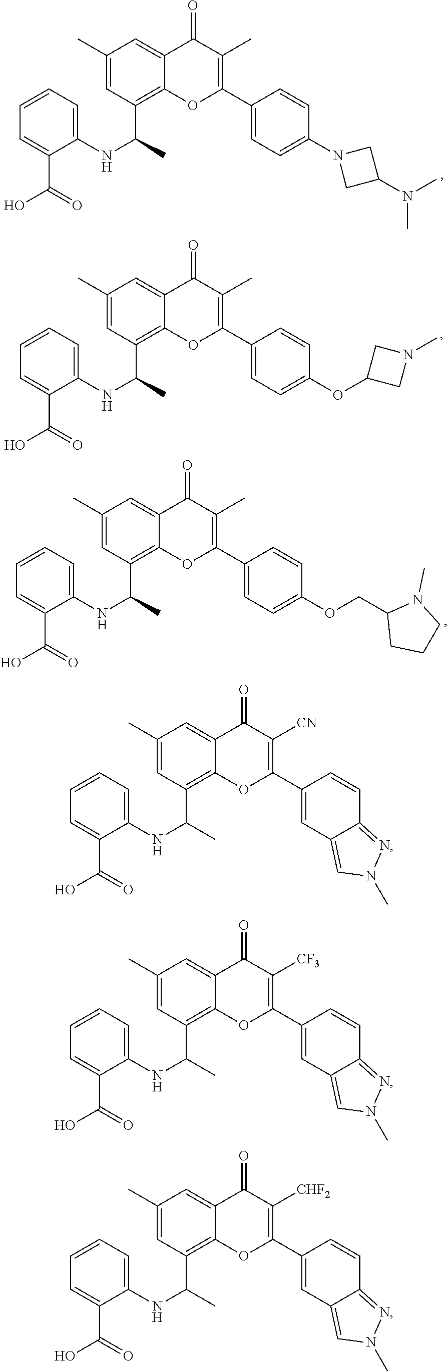 [SMILES: Cc1cc([C@@H](C)Nc2ccccc2C(=O)O)c2oc(-c3ccc(N4CC(N(C)C)C4)cc3)c(C)c(=O)c2c1 ; Cc1cc([C@@H](C)Nc2ccccc2C(=O)O)c2oc(-c3ccc(OC4CN(C)C4)cc3)c(C)c(=O)c2c1 ; Cc1cc([C@@H](C)Nc2ccccc2C(=O)O)c2oc(-c3ccc(OCC4CCCN4C)cc3)c(C)c(=O)c2c1 ; Cc1cc(C(C)Nc2ccccc2C(=O)O)c2oc(-c3ccc4nn(C)cc4c3)c(C#N)c(=O)c2c1 ; Cc1cc(C(C)Nc2ccccc2C(=O)O)c2oc(-c3ccc4nn(C)cc4c3)c(C(F)(F)F)c(=O)c2c1 ; Cc1cc(C(C)Nc2ccccc2C(=O)O)c2oc(-c3ccc4nn(C)cc4c3)c(C(F)F)c(=O)c2c1]

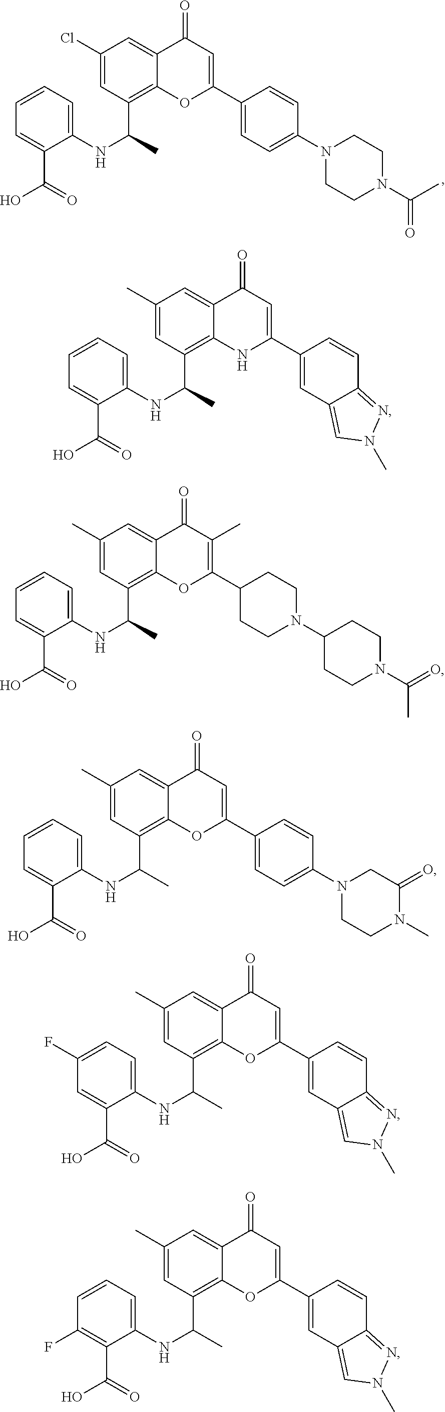 [SMILES: CC(=O)N1CCN(c2ccc(-c3cc(=O)c4cc(Cl)cc([C@@H](C)Nc5ccccc5C(=O)O)c4o3)cc2)CC1 ; Cc1cc([C@@H](C)Nc2ccccc2C(=O)O)c2[nH]c(-c3ccc4nn(C)cc4c3)cc(=O)c2c1 ; CC(=O)N1CCC(N2CCC(c3oc4c([C@@H](C)Nc5ccccc5C(=O)O)cc(C)cc4c(=O)c3C)CC2)CC1 ; Cc1cc(C(C)Nc2ccccc2C(=O)O)c2oc(-c3ccc(N4CCN(C)C(=O)C4)cc3)cc(=O)c2c1 ; Cc1cc(C(C)Nc2ccc(F)cc2C(=O)O)c2oc(-c3ccc4nn(C)cc4c3)cc(=O)c2c1 ; Cc1cc(C(C)Nc2cccc(F)c2C(=O)O)c2oc(-c3ccc4nn(C)cc4c3)cc(=O)c2c1]

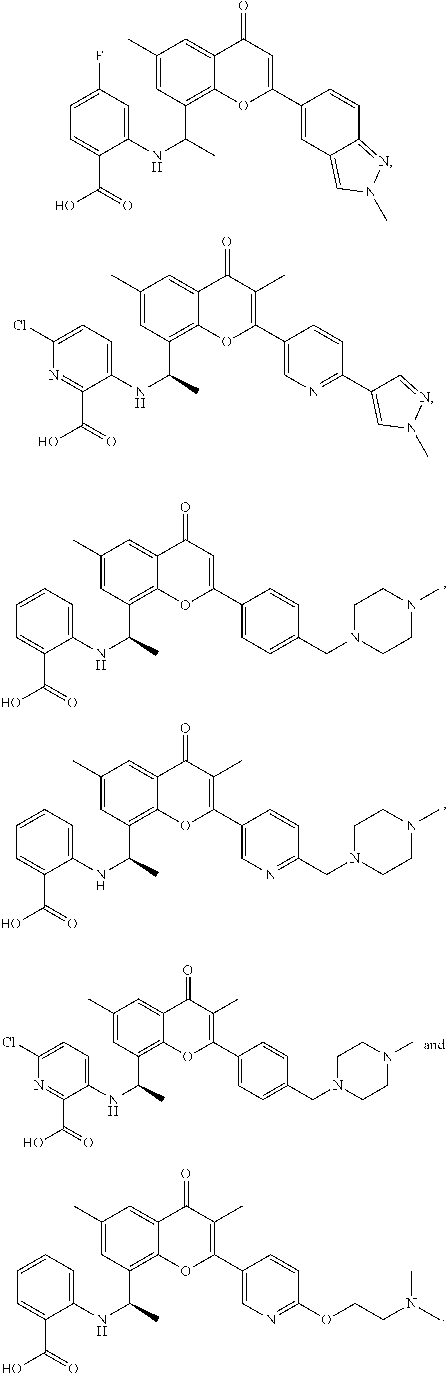 [SMILES: Cc1cc(C(C)Nc2cc(F)ccc2C(=O)O)c2oc(-c3ccc4nn(C)cc4c3)cc(=O)c2c1 ; Cc1cc([C@@H](C)Nc2ccc(Cl)nc2C(=O)O)c2oc(-c3ccc(-c4cnn(C)c4)nc3)c(C)c(=O)c2c1 ; Cc1cc([C@@H](C)Nc2ccccc2C(=O)O)c2oc(-c3ccc(CN4CCN(C)CC4)cc3)cc(=O)c2c1 ; Cc1cc([C@@H](C)Nc2ccccc2C(=O)O)c2oc(-c3ccc(CN4CCN(C)CC4)nc3)c(C)c(=O)c2c1 ; Cc1cc([C@@H](C)Nc2ccc(Cl)nc2C(=O)O)c2oc(-c3ccc(CN4CCN(C)CC4)cc3)c(C)c(=O)c2c1 ; Cc1cc([C@@H](C)Nc2ccccc2C(=O)O)c2oc(-c3ccc(OCCN(C)C)nc3)c(C)c(=O)c2c1]

14. A compound of formula (VI), a stereoisomer thereof, or a pharmaceutically acceptable salt, solvates and prodrugs thereof: 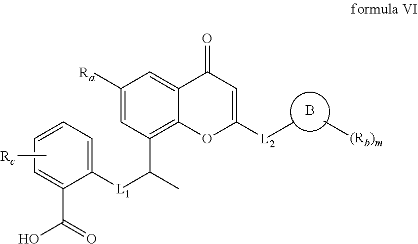
Wherein,
L₁ is selected from —O—(CHR₁)ₚ—, —N(R₁₁)—(CHR₁)ₚ—;
R₁ is identical or different and each is independently selected from hydrogen, deuterium, C₁-C₆ alkyl, C₁-C₆ haloalkyl, C₁-C₆ alkoxy, C₁-C₆ haloalkoxy, halogen, hydroxy, cyano;
R₁₁ is identical or different and each is independently selected from hydrogen, C₁-C₆ alkyl, C₁-C₆ haloalkyl;
Rₐ is independently selected from the group consisting of hydrogen, deuterium, C₁-C₆ alkyl, deuterated C₁-C₆ alkyl, C₁-C₆ haloalkyl, C₁-C₆ alkoxy, C₁-C₆ haloalkoxy, halogen, amino, nitro, hydroxy, cyano, —C(O)N(Rₐₐ)₂, C₃-C₈ cycloalkyl, 4 to 7 membered heterocyclyl, C₆-C₁₀ aryl and 5 to 10 membered heteroaryl, wherein the C₃-C₈ cycloalkyl, 4 to 7 membered heterocyclyl, C₆-C₁₀ aryl and 5 to 10 membered heteroaryl are each optionally further substituted by one or more deuterium, C₁-C₆ alkyl, halogen, hydroxy, amino, nitro, cyano, ester group, C₁-C₆ alkoxy;
Each Rₐₐ is identical or different and is each independently selected from hydrogen or C₁-C₆ alkyl, wherein the C₁-C₆ alkyl optionally further substituted by one or more substituents selected from the group consisting of deuterium, halogen, hydroxy, amino, —N(C₁-C₆ alkyl)₂;
Or two Rₐₐ together with the atoms to which they are attached can form a 4 to 7 membered heterocycle comprising 1-2 heteroatoms selected from O, N, and S, wherein optionally substituted with one or more one or more oxo, halogen, —CN, —OH, —NH₂, C₁-C₆ alkyl, C₁-C₆ haloalkyl or C₁-C₆ alkoxy;
While L₂ is a bond or S;
Ring B is independently selected from 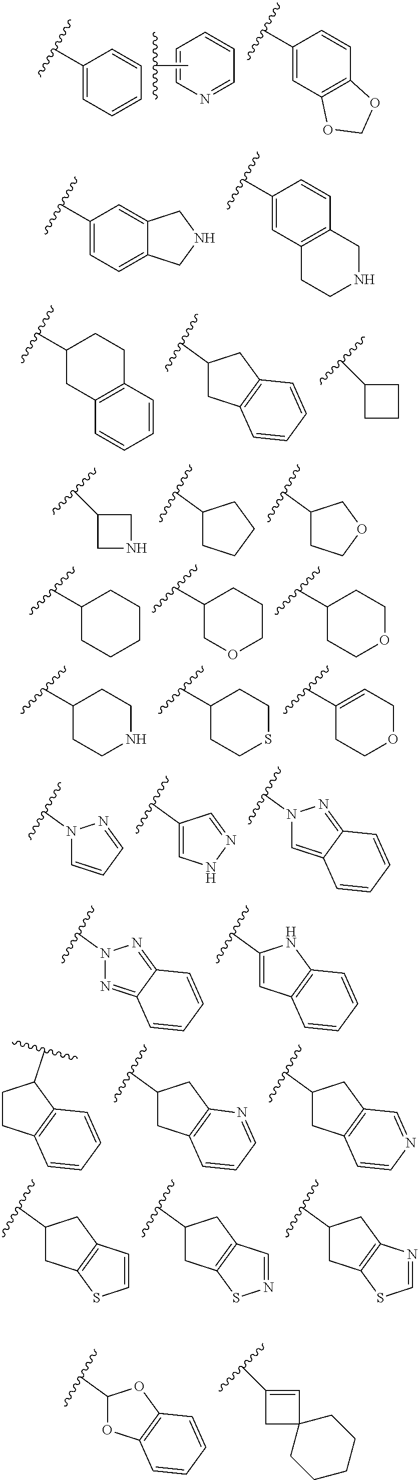
While L₂ is CR₂R₂′;
Each R₂ and R₂′ is independently selected from hydrogen, C₁-C₆ alkyl, C₁-C₆ alkoxyl or C₁-C₆ haloalkyl;
Ring B is independently selected from: 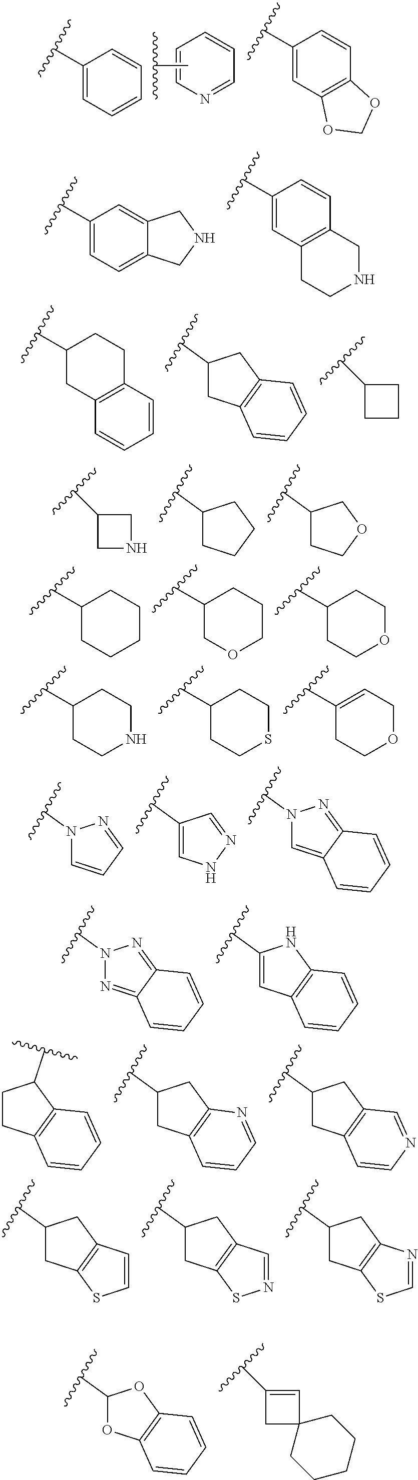 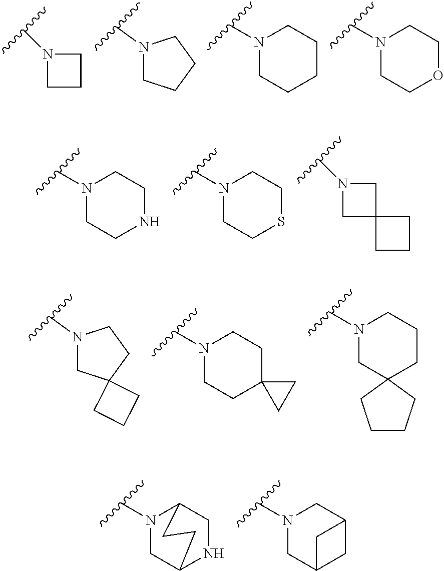
Each R_{b} is identical or different and each is independently selected from the group consisting of hydrogen, oxo, halogen, hydroxy, amino, nitro, cyano, ester group, C₁-C₆ alkyl, C₁-C₆ alkoxy, C₃-C₈ cycloalkyl and 4 to 7 membered heterocyclyl, wherein the C₁-C₆ alky, C₃-C₈ cycloalkyl and 4 to 7 membered heterocyclyl are each optionally further substituted by one or more substituents selected from the group consisting of deuterium, halogen, hydroxy, amino, nitro, cyano, ester group, carboxyl, alkoxy, hydroxyalkyl;
R_{c} is identical or different and is each independently selected from the group consisting of hydrogen, deuterium, C₁-C₆ alkyl, deuterated C₁-C₆ alkyl, C₁-C₆ haloalkyl, C₁-C₆ alkoxy, C₁-C₆ haloalkoxy, —S(O)₂-alkyl, halogen, amino, nitro, hydroxy, cyano, C₃-C₈ cycloalkyl and 4 to 7 membered heterocyclyl, wherein the C₃-C₈ cycloalkyl and 4 to 7 membered heterocyclyl are each optionally further substituted by one or more deuterium, alkyl, halogen, hydroxy, amino, nitro, cyano, ester group, alkoxy;
m is an integer of 0, 1, 2, 3, 4 or 5;
p is an integer of 0, 1 or 2.

15. The compound according to claim 14, a stereoisomer thereof, or a pharmaceutically acceptable salt, solvates and prodrugs thereof, selected from

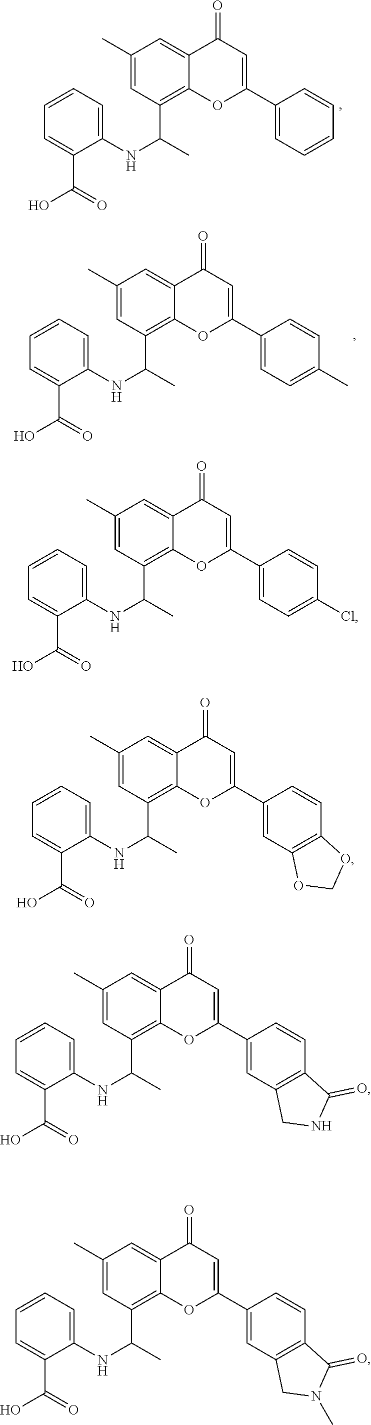 [SMILES: Cc1cc(C(C)Nc2ccccc2C(=O)O)c2oc(-c3ccccc3)cc(=O)c2c1 ; Cc1ccc(-c2cc(=O)c3cc(C)cc(C(C)Nc4ccccc4C(=O)O)c3o2)cc1 ; Cc1cc(C(C)Nc2ccccc2C(=O)O)c2oc(-c3ccc(Cl)cc3)cc(=O)c2c1 ; Cc1cc(C(C)Nc2ccccc2C(=O)O)c2oc(-c3ccc4c(c3)OCO4)cc(=O)c2c1 ; Cc1cc(C(C)Nc2ccccc2C(=O)O)c2oc(-c3ccc4c(c3)CNC4=O)cc(=O)c2c1 ; Cc1cc(C(C)Nc2ccccc2C(=O)O)c2oc(-c3ccc4c(c3)CN(C)C4=O)cc(=O)c2c1]

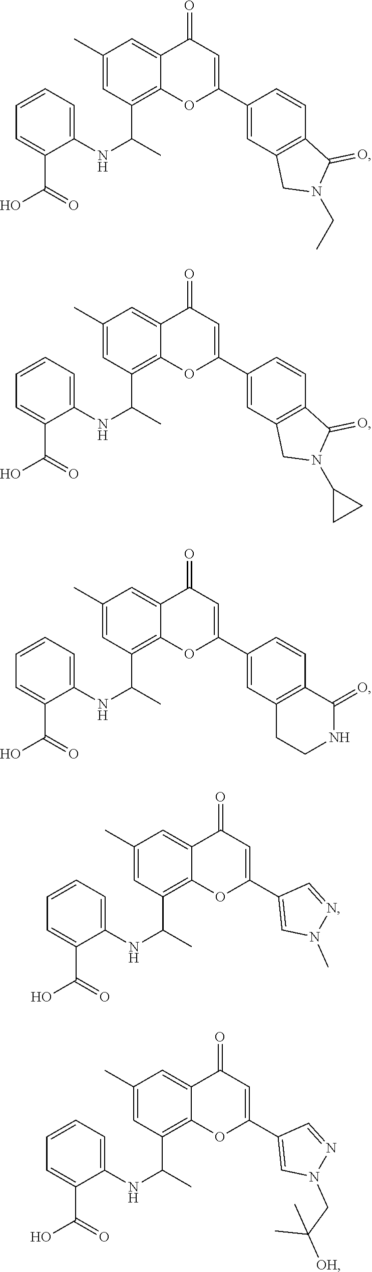 [SMILES: CCN1Cc2cc(-c3cc(=O)c4cc(C)cc(C(C)Nc5ccccc5C(=O)O)c4o3)ccc2C1=O ; Cc1cc(C(C)Nc2ccccc2C(=O)O)c2oc(-c3ccc4c(c3)CN(C3CC3)C4=O)cc(=O)c2c1 ; Cc1cc(C(C)Nc2ccccc2C(=O)O)c2oc(-c3ccc4c(c3)CCNC4=O)cc(=O)c2c1 ; Cc1cc(C(C)Nc2ccccc2C(=O)O)c2oc(-c3cnn(C)c3)cc(=O)c2c1 ; Cc1cc(C(C)Nc2ccccc2C(=O)O)c2oc(-c3cnn(CC(C)(C)O)c3)cc(=O)c2c1]

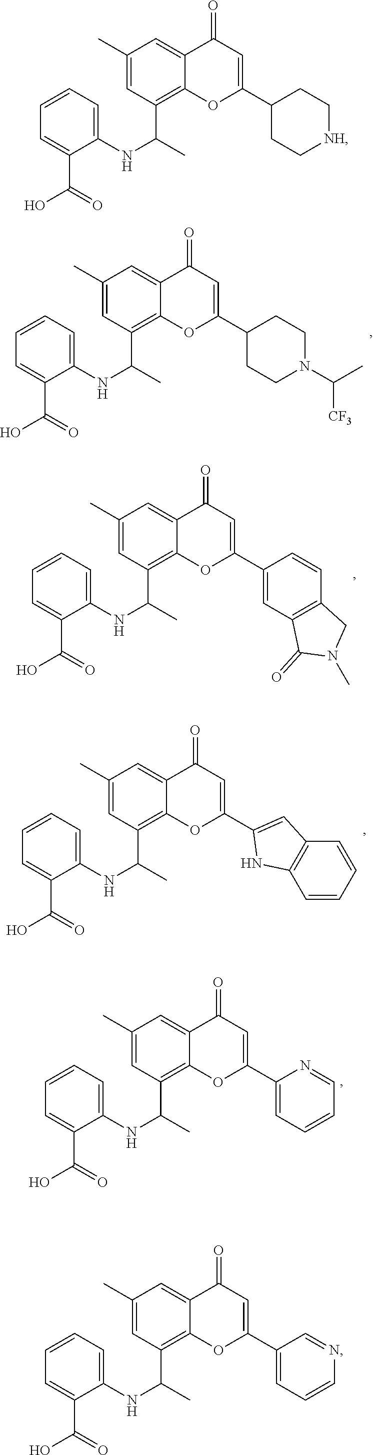 [SMILES: Cc1cc(C(C)Nc2ccccc2C(=O)O)c2oc(C3CCNCC3)cc(=O)c2c1 ; Cc1cc(C(C)Nc2ccccc2C(=O)O)c2oc(C3CCN(C(C)C(F)(F)F)CC3)cc(=O)c2c1 ; Cc1cc(C(C)Nc2ccccc2C(=O)O)c2oc(-c3ccc4c(c3)C(=O)N(C)C4)cc(=O)c2c1 ; Cc1cc(C(C)Nc2ccccc2C(=O)O)c2oc(-c3cc4ccccc4[nH]3)cc(=O)c2c1 ; Cc1cc(C(C)Nc2ccccc2C(=O)O)c2oc(-c3ccccn3)cc(=O)c2c1 ; Cc1cc(C(C)Nc2ccccc2C(=O)O)c2oc(-c3cccnc3)cc(=O)c2c1]

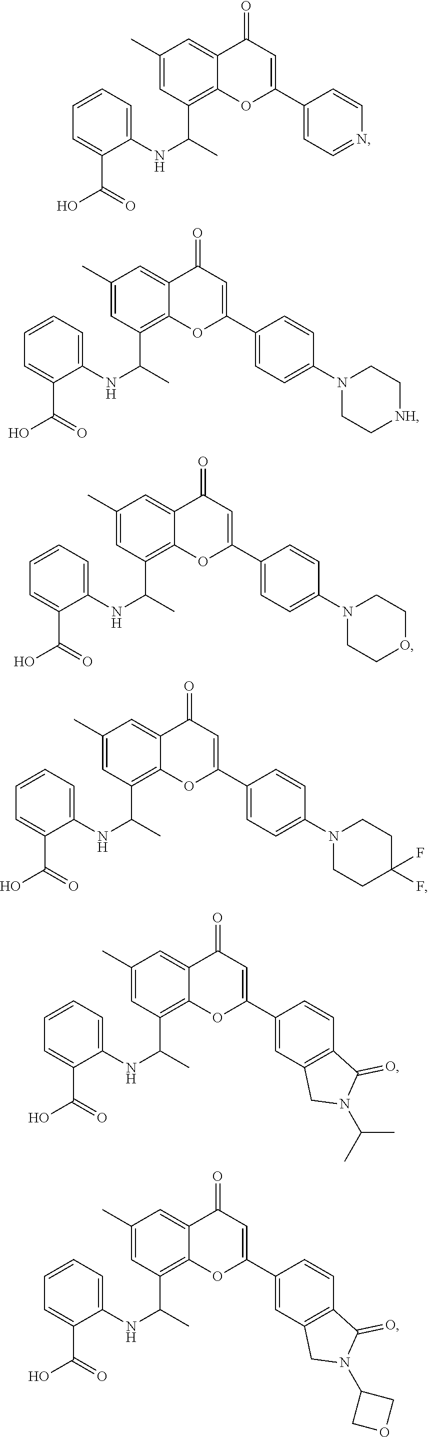 [SMILES: Cc1cc(C(C)Nc2ccccc2C(=O)O)c2oc(-c3ccncc3)cc(=O)c2c1 ; Cc1cc(C(C)Nc2ccccc2C(=O)O)c2oc(-c3ccc(N4CCNCC4)cc3)cc(=O)c2c1 ; Cc1cc(C(C)Nc2ccccc2C(=O)O)c2oc(-c3ccc(N4CCOCC4)cc3)cc(=O)c2c1 ; Cc1cc(C(C)Nc2ccccc2C(=O)O)c2oc(-c3ccc(N4CCC(F)(F)CC4)cc3)cc(=O)c2c1 ; Cc1cc(C(C)Nc2ccccc2C(=O)O)c2oc(-c3ccc4c(c3)CN(C(C)C)C4=O)cc(=O)c2c1 ; Cc1cc(C(C)Nc2ccccc2C(=O)O)c2oc(-c3ccc4c(c3)CN(C3COC3)C4=O)cc(=O)c2c1]

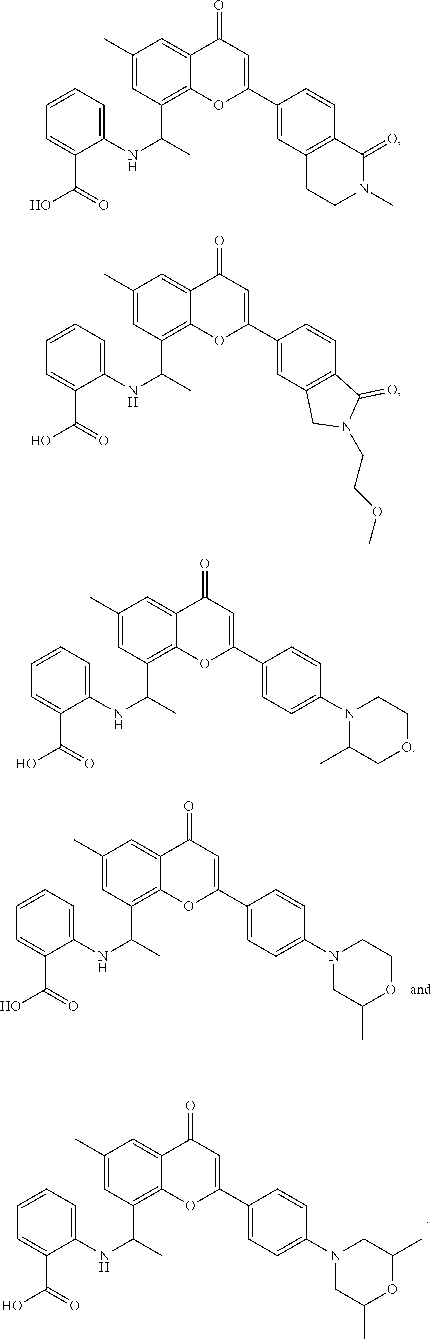 [SMILES: Cc1cc(C(C)Nc2ccccc2C(=O)O)c2oc(-c3ccc4c(c3)CCN(C)C4=O)cc(=O)c2c1 ; COCCN1Cc2cc(-c3cc(=O)c4cc(C)cc(C(C)Nc5ccccc5C(=O)O)c4o3)ccc2C1=O ; Cc1cc(C(C)Nc2ccccc2C(=O)O)c2oc(-c3ccc(N4CCOCC4C)cc3)cc(=O)c2c1 ; Cc1cc(C(C)Nc2ccccc2C(=O)O)c2oc(-c3ccc(N4CCOC(C)C4)cc3)cc(=O)c2c1 ; "and" ; Cc1cc(C(C)Nc2ccccc2C(=O)O)c2oc(-c3ccc(N4CC(C)OC(C)C4)cc3)cc(=O)c2c1]

16. A compound of formula (VIII), a stereoisomer thereof, or a pharmaceutically acceptable salt, solvates and prodrugs thereof: 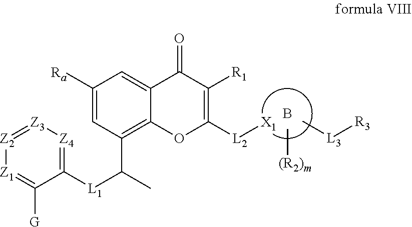
Wherein:
G is selected from —C(O) R_{g}, —C(O)OR_{g}, —OC(O)R_{g}, —C(O)N(R_{g})₂, —C(O)NH(OR_{g}), —N(R_{g})C(O)R_{g}, —S(O)₂R_{g}, —S(O)₂OR_{g}, —OS(O)₂R_{g}, —S(O)₂N(R)₂, —N((R_{g})S(O)₂R_{g}, —P(O)(R_{g})₂, —P(O)(OR_{g})₂, —OP(O)OR_{g}, —B(OR_{g})₂, —C(O)N(R_{g})—S(O)₂R_{g} and —S(O)₂N(R_{g})—C(O)R_{g}, C₃-C₈ cycloalkyl, 4 to 10 membered heterocyclyl, C₆-C₁, aryl and 5 to 10 membered heteroaryl, wherein C₃-C₈ cycloalkyl, 4 to 10 membered heterocyclyl, C₆-C₁₀ aryl and 5 to 10 membered heteroaryl are each optionally further substituted by one or more hydrogen, deuterium, oxo, halogen, hydroxy, amino, nitro, cyano, ester group, carboxyl, C₁-C₆ alkyl, C₁-C₆ hydroxyalkyl, C₁-C₆ haloalkyl and C₁-C₆ alkoxy.
Each R_{g} is independently selected from H, C₁-C₆ alkyl, C₂-C₆ alkenyl, C₂-C₆ alkynyl, C₁-C₆ haloalkyl, C₁-C₆ alkoxy, C₃-C₅ cycloalkyl, 4 to 10 membered heterocyclyl, C₆-C₁₀ aryl and 5 to 10 membered heteroaryl, wherein C₃-C₈ cycloalkyl, 4 to 10 membered heterocyclyl, C₆-C₁₀ aryl and 5 to 10 membered heteroaryl are each optionally further substituted by one or more hydrogen, deuterium, oxo, halogen, hydroxy, amino, nitro, cyano, ester group, carboxyl, C₁-C₆ alkyl, C₁-C₆ hydroxyalkyl, C₁-C₆ haloalkyl and C₁-C₆ alkoxy.
Z₁, Z₂, Z₃, Z₄, L₁, L₂, L₃, Rₐ, R₁, R₂, R₃, X₁, Ring B and in are defined as in claim 1.

17. The compound according to claim 16, a stereoisomer thereof, or a pharmaceutically acceptable salt, solvates and prodrugs thereof, selected from

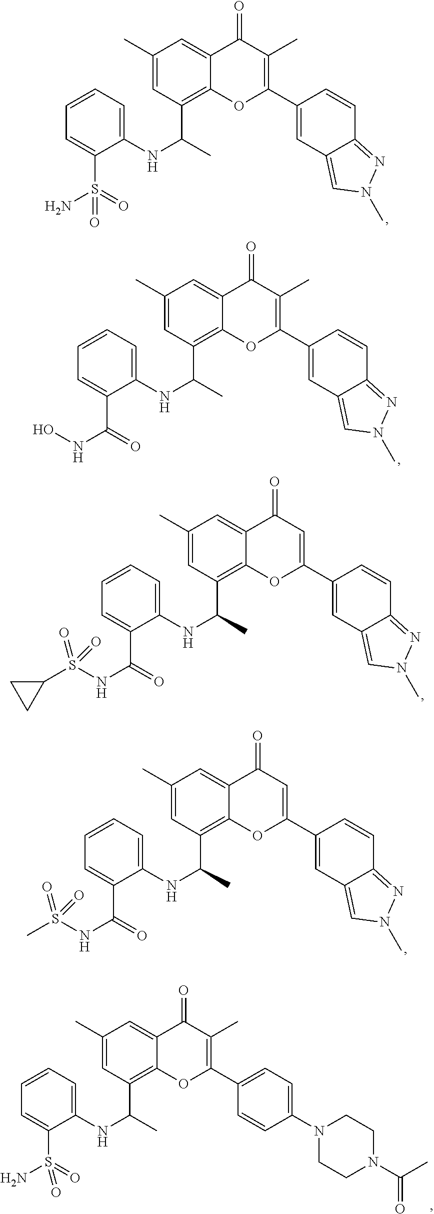

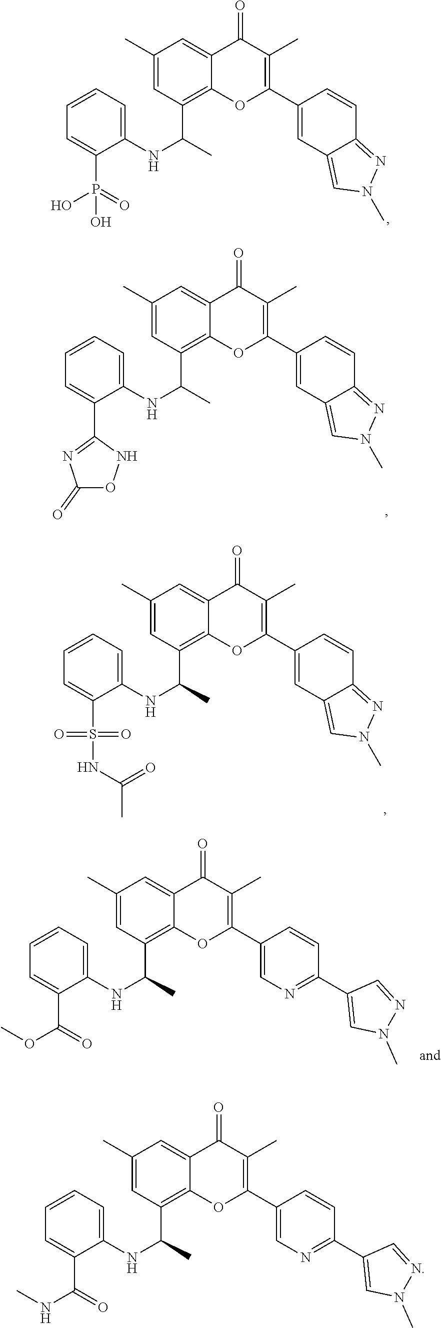

18. A pharmaceutical composition comprising a compound of any one of claims 1-17, or a stereoisomer thereof, or a pharmaceutically acceptable salt, solvates and prodrugs thereof, and one or more pharmaceutically acceptable carriers.

19. Use of the compound of a compound of any one of claims 1-17, a stereoisomer thereof-, or a pharmaceutically acceptable salt, solvates and prodrugs thereof in the preparation of a PI3Kα inhibitor medicament.

20. A method of inhibiting PI3Kα activity, said method comprising administering to a subject a compound of any one of claims 1-17, or a stereoisomer thereof, or a pharmaceutically acceptable salt, solvates and prodrugs thereof, or a pharmaceutical composition of claim 18.

21. A method of treating a disease or disorder associated with inhibition of PI3Kα, said method comprising administering to a subject in need thereof a therapeutically effective amount of a compound of any one of claims 1-17, a stereoisomer thereof, or a pharmaceutically acceptable salt, solvates and prodrugs thereof, or a pharmaceutical composition of claim 18.

22. A method for treating a cancer in a patient, said method comprising administering to the patient a therapeutically effective amount of the compound of any one of claims 1-17, or a stereoisomer thereof, or a pharmaceutically acceptable salt, solvates and prodrugs thereof, or a pharmaceutical composition of claim 18.
